(19) United States
(12) Patent Application Publication
(10) Pub. No.: US 2024/0270750 A1
(43) Pub. Date: Aug. 15, 2024

(54) IMIDAZOLE-CONTAINING INHIBITORS OF ALK2 KINASE

(71) Applicant: BioCryst Pharmaceuticals, Inc., Durham, NC (US)

(72) Inventors: Pravin L. Kotian, Hoover, AL (US); Yarlagadda S. Babu, Birmingham, AL (US); Weihe Zhang, Vestavia Hills, AL (US); Wei Lv, Hoover, AL (US); Peng-Cheng Lu, Vestavia Hills, AL (US); Andrew E. Spaulding, Hoover, AL (US); Krishnan Raman, Birmingham, AL (US)

(21) Appl. No.: 18/562,115

(22) PCT Filed: May 24, 2022
(86) PCT No.: PCT/US22/30690

§ 371 Date: Nov. 17, 2023

Related U.S. Application Data
(60) Provisional application No. 63/192,822, filed on May 25, 2021.

(51) Int. Cl. C07D 487/04; A61K 31/517; A61K 31/519; A61K 31/52; A61K 31/53; C07D 403/12; C07D 471/04; C07D 473/34; C07D 491/048; C07D 495/04; C07D 513/04

(52) U.S. Cl. CPC C07D 487/04; A61K 31/517; A61K 31/519; A61K 31/52; A61K 31/53; C07D 403/12; C07D 471/04; C07D 473/34; C07D 491/048; C07D 495/04; C07D 513/04

(57) ABSTRACT

Disclosed are compounds of formula I, II, III, and IV, and pharmaceutically acceptable salts thereof. The compounds are inhibitors of ALK2 kinase. Also provided are pharmaceutical compositions comprising a compound of formula I, II, III, or IV, or pharmaceutically acceptable salt thereof, and methods involving use of the compounds or pharmaceutically acceptable salts thereof and compositions in the treatment and prevention of various diseases and conditions, such as fibrodysplasia ossificans progressiva.

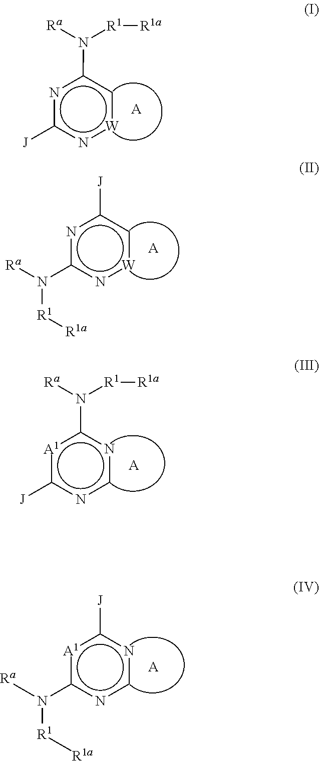

### RELATED APPLICATIONS

This application claims the benefit of priority to U.S. Provisional Patent Application Ser. No. 63/192,822, filed May 25, 2021.

### BACKGROUND OF THE INVENTION

A single mutation (R206H) within the kinase domain of one (ACVR1/ALK2) of the four human bone morphogenetic protein (BMP) receptors has been linked to a catastrophic disorder of secondary (heterotopic) bone formation. As a result of the mutation, all children presenting with features of classic Fibrodysplasia Ossificans Progressiva (FOP) eventually become encased in, and their movement blocked by, a second heterotopic skeleton. The disorder has long been associated with dysregulation of BMP signaling in soft tissues (skeletal muscle, tendon, ligament, fascia) that were transformed into ribbons, sheets and plates of heterotopic bone via an endochondral process. In addition to the common R206H mutation linked to the classic form of FOP, other dysregulating mutations have been identified in ACVR1/ALK2 that lead to atypical and variant forms of FOP. Further, compounds effective in regulating BMP signaling based on their ability to inhibit ALK2 have been shown also to inhibit kinases from multiple signaling pathways.

Thus, there remains a need for additional compounds that inhibit the ALK2 kinase which will be suitable for various important therapeutic applications.

### SUMMARY OF THE INVENTION

In certain aspects, the invention provides a compound of formula (I) or formula (II):

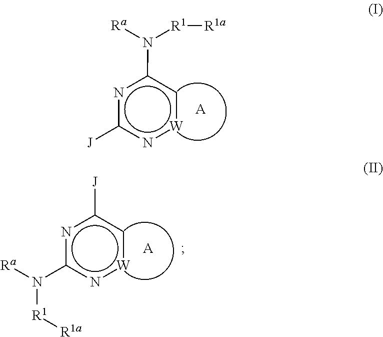

or a pharmaceutically acceptable salt thereof;
wherein:
A is a fused optionally substituted aromatic ring, heteroaromatic ring, cycloalkyl ring, cycloalkenyl ring, heterocycloalkyl ring, or heterocycloalkenyl ring;
W is C or N;
R^{a} represents H or alkyl;
R¹ represents heteroarylene;
R^{1a} represents H or optionally substituted —C(O)alkyl, —C(O)aryl, —C(O)heteroaryl, —C(O)O(alkyl), —C(O)(heterocyclyl), —C(O)NR^{x}R^{y}, alkyl, cycloalkyl, cycloalkenyl, heterocycloalkyl, aryl, or heteroaryl;
J represents optionally substituted alkyl, alkenyl, haloalkyl, hydroxyalkyl, alkoxyalkyl, aminoalkyl, cycloalkyl, (cycloalkyl)alkyl, heterocycloalkyl, (heterocycloalkyl)alkyl, or heterocycloalkenyl;
   further wherein when J is heterocycloalkyl or heterocycloalkenyl, the point of attachment in J to the rest of the compound is a carbon atom; and
   R^{x} and R^{y} each independently represent H, alkyl, aralkyl, heteroaralkyl, aryl, heteroaryl, cycloalkyl, heterocycloalkyl, (cycloalkyl)alkyl, (heterocycloalkyl)alkyl, or hydroxyalkyl.

In further embodiments, the invention provides a compound represented by formula (III) or formula (IV).

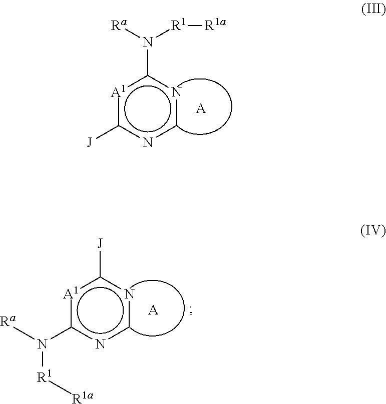

or a pharmaceutically acceptable salt thereof;
wherein:
A is a fused optionally substituted aromatic ring, heteroaromatic ring, cycloalkyl ring, cycloalkenyl ring, heterocycloalkyl ring, or heterocycloalkenyl ring;
A¹ is CH or N;
R^{a} represents H or alkyl;
R¹ represents heteroarylene;
R^{1a} represents H or optionally substituted —C(O)alkyl, —C(O)aryl, —C(O)heteroaryl, —C(O)O(alkyl), —C(O)(heterocyclyl), —C(O)NR^{x}R^{y}, alkyl, cycloalkyl, cycloalkenyl, heterocycloalkyl, aryl, or heteroaryl;
J represents optionally substituted alkyl, alkenyl, haloalkyl, hydroxyalkyl, alkoxyalkyl, cycloalkyl, (cycloalkyl)alkyl, heterocycloalkyl, (heterocycloalkyl)alkyl, or heterocycloalkenyl; and
   R^{x} and R^{y} each independently represent H, alkyl, aralkyl, heteroaralkyl, aryl, heteroaryl, cycloalkyl, heterocycloalkyl, (cycloalkyl)alkyl, (heterocycloalkyl)alkyl, or hydroxyalkyl.

In certain aspects, the invention provides a pharmaceutical composition, comprising a compound of the invention, or a pharmaceutically acceptable salt thereof; and a pharmaceutically acceptable carrier.

In certain aspects, the invention provides methods of inhibiting ALK2 kinase, comprising administering to a subject in need thereof an effective amount of a compound of the invention, or a pharmaceutically acceptable salt thereof.

The present invention also provides methods of treating fibrodysplasia ossificans progressiva, comprising administering to a subject in need thereof a therapeutically effective amount of a compound of the invention, or a pharmaceutically acceptable salt thereof.

In further aspects, the invention provides methods of treating cancer, comprising administering to a subject in need thereof a therapeutically effective amount of a compound of the invention, or a pharmaceutically acceptable salt thereof. In certain embodiments, the cancer is a glioma, such as diffuse intrinsic pontine glioma.

In further aspects, the invention provides a method of treating anemia associated with high hepcidin, Iron Refractory Iron Deficiency Anemia (IRIDA), anemia of chronic diseases, cancer-related anemia, chemotherapy-associated anemia, anemia of inflammation, or hepcidin-producing adenoma, comprising administering to a subject in need thereof a therapeutically effective amount of a compound of the invention, or a pharmaceutically acceptable salt thereof.

In further aspects, the invention provides a method of treating spondyloarthritis (SpA) comprising administering to a subject in need thereof a therapeutically effective amount of a compound of the invention, or a pharmaceutically acceptable salt thereof.

### DETAILED DESCRIPTION

Provided herein are compounds of formulae (I), (II), (III), and (IV), and pharmaceutically acceptable salts thereof, that are useful for inhibiting ALK2 kinase, and useful in the treatment or prevention of a disease or condition that would benefit from inhibition of ALK2 kinase. For example, the disclosed inhibitors of ALK2 kinase are useful in therapeutic methods and compositions suitable for use in treating cancer or fibrodysplasia ossificans progressiva.

### Definitions

The articles “a” and “an” are used herein to refer to one or to more than one (i.e., to at least one) of the grammatical object of the article. By way of example, “an element” means one element or more than one element.

The term “heteroatom” is art-recognized and refers to an atom of any element other than carbon or hydrogen. Illustrative heteroatoms include boron, nitrogen, oxygen, phosphorus, sulfur and selenium, and alternatively oxygen, nitrogen or sulfur.

The term “alkyl” as used herein is a term of art and refers to saturated aliphatic groups, including straight-chain alkyl groups, branched-chain alkyl groups, cycloalkyl groups, alkyl substituted cycloalkyl groups, and (cycloalkyl)alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C₁₀ alkyl group. In certain embodiments, the term “alkyl” refers to a C₁-C₆ alkyl group, for example a C₁-C₆ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “cycloalkyl” means mono- or bicyclic saturated carbocyclic rings, each having from 3 to 12 carbon atoms. Certain cycloalkyls have from 5-12 carbon atoms in their ring structure, and may have 6-10 carbons in the ring structure. Preferably, cycloalkyl is (C₃-C₇)cycloalkyl, which represents a monocyclic saturated carbocyclic ring, having from 3 to 7 carbon atoms. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems include bridged monocyclic rings and fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3).

Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocycloalkyl, a monocyclic heterocycloalkenyl, or a monocyclic heteroaryl. The bridged or fused bicyclic cycloalkyl is attached to the parent molecular moiety through any carbon atom contained within the monocyclic cycloalkyl ring. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocycloalkyl, a or 6 membered monocyclic heterocycloalkenyl, or a 5 or 6 membered monocyclic heteroaryl, wherein the fused bicyclic cycloalkyl is optionally substituted.

The term “cycloalkylene” as used herein refers to a divalent cycloalkyl group. In some embodiments, a cycloalkylene may be fused to an arylene or heteroarylene group; i.e., a cycloalkylene may be bonded at two adjacent positions to an arylene or heteroarylene group.

In such embodiments, the cycloalkylene is saturated at all atoms except the atoms that are fused to the arylene group.

The term “(cycloalkyl)alkyl” as used herein refers to an alkyl group substituted with one or more cycloalkyl groups. An example of (cycloalkyl)alkyl is cyclohexylmethyl group.

The term “cycloalkenyl” as used herein refers to a cycloalkyl group as defined above that additionally contains at least one carbon-carbon double bond. In certain embodiments, the cycloalkenyl is a mono- or bicyclic carbocyclic ring having at least one carbon-carbon double bond and containing from 3 to 12 carbon atoms. For avoidance of doubt, a cycloalkenyl group is not aromatic. Representative examples of cycloalkenyl include, but are not limited to, cyclohexenyl and cyclopentenyl.

The term “cycloalkynyl” as used herein refers to a cycloalkyl group as defined above that additionally contains at least one carbon-carbon triple bond. In certain embodiments, the cycloalkynyl is a mono- or bicyclic carbocyclic ring having at least one carbon-carbon triple bond and containing from 3 to 12 carbon atoms. For avoidance of doubt, a cycloalkynyl group is not aromatic.

The term “cycloalkenylene” as used herein refers to a divalent cycloalkenyl group. In some embodiments, a cycloalkenylene may be fused to an arylene or heteroarylene group, i.e., a cycloalkenylene may be bonded at two adjacent positions to an arylene or heteroarylene group. In such embodiments, the cycloalkenylene contains at least one saturated carbon atom and at least one carbon-carbon double bond in addition to the atoms that are fused to the arylene group.

The term “heterocycloalkyl” as used herein refers to a radical of a non-aromatic ring system, including, but not limited to, monocyclic, bicyclic, and tricyclic rings, which can be completely saturated or which can contain one or more units of unsaturation, wherein for the avoidance of doubt, the degree of unsaturation does not result in an aromatic ring system, and having 3 to 12 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithictyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, trithianyl, and 2-azobicyclo[3.1.0]hexane. A heterocycloalkyl group may be optionally substituted by one or more substituents as described below. In certain embodiments, for example in substituent J, the heterocycloalkyl is attached to the rest of the molecule through a carbon atom in the heterocycloalkyl group, i.e., not through a heteroatom, such as a nitrogen atom, in the heterocycloalkyl group.

The term “heterocycloalkylene” as used herein refers to a divalent heterocycloalkyl group. In some embodiments, a heterocycloalkylene may be fused to an arylene or heteroarylene group; i.e., a heterocycloalkylene may be bonded at two adjacent positions to an arylene or heteroarylene group. In such embodiments, the heterocycloalkylene is saturated at all atoms except the atoms that are fused to the arylene group.

The term “heterocycloalkenyl” as used herein refers to a heterocycloalkyl group, as defined above, that additionally contains at least one carbon-carbon double bond. For avoidance of doubt, a heterocycloalkenyl group is not aromatic.

The term “(heterocycloalkyl)alkyl” as used herein refers to an alkyl group substituted with one or more heterocycloalkyl (i.e., heterocyclyl) groups.

The term “heterocycloalkynyl” as used herein refers to a heterocycloalkyl group, as defined above, that additionally contains at least one carbon-carbon triple bond. For avoidance of doubt, a heterocycloalkynyl group is not aromatic.

The term “heterocycloalkenylene” as used herein refers to a divalent heterocycloalkenyl group. In some embodiments, a heterocycloalkenylene may be fused to an arylene or heteroarylene group; i.e., a heterocycloalkenylene may be bonded at two adjacent positions to an arylene or heteroarylene group. In such embodiments, the heterocycloalkenylene contains at least one carbon-carbon double bond in addition to the atoms that are fused to the arylene group.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, I-propynyl, 2-propynyl, 3-butynyl, 2-pentynyl, and 1-butynyl.

The term “alkylene” is art-recognized, and as used herein pertains to a diradical obtained by removing two hydrogen atoms of an alkyl group, as defined above. In one embodiment an alkylene refers to a disubstituted alkane, i.e., an alkane substituted at two positions with substituents such as those described below. That is, in one embodiment, a “substituted alkyl” is an “alkylene”.

The term “amino” is a term of art and as used herein refers to both unsubstituted and substituted amines, e.g., a moiety that may be represented by the general formulas:

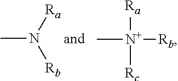

wherein Rₐ, R_{b}, and R_{c} each independently represent a hydrogen, —(CH₂)ₓ—R_{d}, —C(O)-alkyl, —C(O)-alkenyl, where the alkyl or alkenyl may be optionally substituted, or optionally substituted alkyl, alkenyl, alkynyl, aryl, heteroaryl, cycloalkyl, cycloalkenyl, heterocycloalkyl, heterocycloalkenyl, (cycloalkyl)alkyl, (heterocycloalkyl)alkyl, arylalkyl, heteroarylalkyl, alkoxyalkyl, or haloalkyl; or R^{a} and R_{b}, taken together with the N atom to to which they are attached form a heterocycle having from 4 to 8 atoms in the ring structure, which may be optionally substituted; R_{d} represents optionally substituted aryl, heteroaryl, cycloalkyl, cycloalkenyl, heterocyclyl or polycyclyl; and x is zero or an integer in the range of 1 to 8. In certain embodiments, only one of Rₐ or R_{b} contains a carbonyl adjacent to the N atom, e.g., Rₐ, R_{b}, and the nitrogen together do not form an imide. In other embodiments, Rₐ and R_{b} (and optionally R_{c}) each independently represent hydrogen, optionally substituted alkyl, optionally substituted alkenyl, or —(CH₂)ₓ—R_{d}. In certain embodiments, the term “amino” refers to —NH₂.

In certain embodiments, the term “alkylamino” refers to —NH(alkyl).

In certain embodiments, the term “dialkylamino” refers to —N(alkyl)₂.

The term “amido”, as used herein, means —NHC(═O)—, wherein the amido group is bound to the parent molecular moiety through the nitrogen. Examples of amido include alkylamido such as CH₃C(═O)N(H)— and CH₃CH₂C(═O)N(H)—.

The term “acyl” is a term of art and as used herein refers to any group or radical of the form RC(O)— where R is any organic group, e.g., alkyl, aryl, heteroaryl, aralkyl, and heteroaralkyl. Representative acyl groups include acetyl, benzoyl, and malonyl.

The term “aminoalkyl” as used herein refers to an alkyl group substituted with one or more one amino groups. In one embodiment, the term “aminoalkyl” refers to an aminomethyl group, i.e., —CH₂NH₂.

The term “aminoacyl” is a term of art and as used herein refers to an acyl group substituted with one or more amino groups.

The term “aminothionyl” is a term of art and as used herein refers to any group or radical of the form RC(S)—, wherein R is any organic group, e.g., alkyl, aryl, heteroaryl, arylalkyl, and heteroarylalkyl.

The term “phosphoryl” is a term of art and as used herein may in general be represented by the formula:

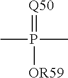

wherein Q50 represents S or O, and R59 represents hydrogen, optional substituted (C₁-C₆) alkyl or optionally substituted aryl; for example, —P(O)(OMe)- or —P(O)(OH)₂. When used to substitute, e.g., an alkyl, the phosphoryl group of the phosphorylalkyl may be represented by the general formulas:

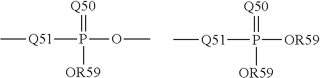

wherein Q50 and R59, each independently, are defined above, and Q51 represents O, S or N; for example, —O—P(O)(OH)OMe or —NH—P(O)(OH)₂. When Q50 is S, the phosphoryl moiety is a “phosphorothioate.”

The term “aminophosphoryl” as used herein refers to a phosphoryl group substituted with at least one amino group, as defined herein; for example, —P(O)(OH)NMe₂.

The term “azide” or “azido”, as used herein, means an —N₃ group.

The term “carbonyl” as used herein refers to —C(═O)—.

The term “thiocarbonyl” as used herein refers to —C(═S)—.

The term “alkylphosphoryl” as used herein refers to a phosphoryl group substituted with at least one alkyl group, as defined herein; for example, —P(O)(OH)Me.

The term “alkylthio” as used herein refers to alkyl-S—. The term “(alkylthio)alkyl” refers to an alkyl group substituted by an alkylthio group.

The term “carboxy”, as used herein, means a —CO₂H group.

The term “aryl” is a term of art and as used herein refers to includes monocyclic, bicyclic and polycyclic aromatic hydrocarbon groups, for example, benzene, naphthalene, anthracene, and pyrene. Typically, an aryl group contains from 6-10 carbon ring atoms (i.e., (C₆-C₁₀)aryl). The aromatic ring may be optionally substituted at one or more ring positions with one or more substituents as described below. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, heterocyclyls, heterocycloalkenyls, and/or heterocycloalkynyls. In certain embodiments, the term “aryl” refers to a phenyl group.

The term “arylene” means a diradical obtained by removing two hydrogen atoms of an aryl group, as defined above. Arylene includes, without limitation, 1,2-phenylene, 1,3-phenylene, and 1,4-phenylene, as depicted below:

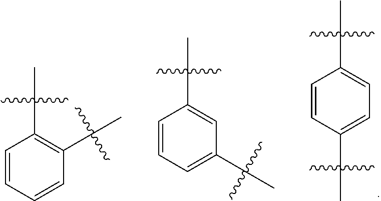 [SMILES: *c1ccccc1* ; *c1cccc(*)c1 ; *c1ccc(*)cc1]

Arylene groups may be optionally substituted at one or more ring positions with one or more substituents, valency permitting, such as the exemplary substituents described below.

The term “heteroaryl” is a term of art and as used herein refers to a monocyclic, bicyclic, and polycyclic aromatic group having 3 to 12 total atoms including one or more heteroatoms such as nitrogen, oxygen, or sulfur in the ring structure. Exemplary heteroaryl groups include azaindolyl, benzo(b)thienyl, benzimidazolyl, benzofuranyl, benzoxazolyl, benzothiazolyl, benzothiadiazolyl, benzotriazolyl, benzoxadiazolyl, furanyl, 1,3-dihydro-2H-imidazol-2-one, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be optionally substituted at one or more ring positions with one or more substituents as described below. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, heterocyclyls, heterocycloalkenyls, and/or heterocycloalkynyls.

The term “heteroarylene” as used herein pertains to a diradical obtained by removing two hydrogen atoms of a heteroaryl group, as defined above. Heteroarylene includes, without limitation, the divalent heteroarylene groups depicted below:

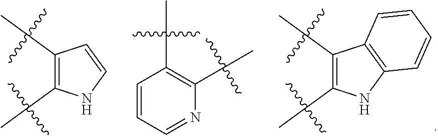

Heterorylene groups may be optionally substituted at one or more ring positions with one or more substituents, valency permitting, such as the exemplary substituents described below.

The term “aralkyl” or “arylalkyl” is a term of art and as used herein refers to an alkyl group substituted with an aryl group, wherein the moiety is appended to the parent molecule through the alkyl group.

The term “heteroaralkyl” or “heteroarylalkyl” is a term of art and as used herein refers to an alkyl group, as defined herein, substituted with a heteroaryl group, as defined herein, appended to the parent molecular moiety through the alkyl group.

The term “alkoxy” as used herein refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “haloalkoxy” as used herein refers to an alkoxy group, as defined herein, wherein some or all of the hydrogens of the alkyl group are replaced with halogen atoms, as defined herein. Representative examples of haloalkoxy include, but are not limited to, —OCF₃.

The term “alkoxyalkyl” as used herein refers to an alkyl group, as defined herein, substituted by an alkoxy group as defined herein.

The term “alkoxycarbonyl” as used herein means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkylcarbonyl”, as used herein, means an alkyl group, as defined herein, appended to the parent molecular moiety through a carbonyl group, represented by —C(═O)—, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “arylcarbonyl”, as used herein, means an aryl group, as defined herein, appended to the parent molecular moiety through a carbonyl group, represented by —C(═O)—, as defined herein. Representative examples of arylcarbonyl include, but are not limited to, benzoyl and (2-pyridinyl)carbonyl.

The term “alkylcarbonyloxy” and “arylcarbonyloxy”, as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkenoxy” or “alkenoxyl” means an alkenyl group, as defined herein, appended to the parent molecular moiety through an oxygen atom. Representative examples of alkenoxyl include, but are not limited to, 2-propen-1-oxyl (i.e., CH₂═CH—CH₂—O—) and vinyloxy (i.e., CH₂═CH—O—).

The term “aryloxy” as used herein means an aryl group, as defined herein, appended to the parent molecular moiety through an oxygen atom.

The term “heteroaryloxy” as used herein means a heteroaryl group, as defined herein, appended to the parent molecular moiety through an oxygen atom.

The term “carbocyclyl” as used herein means a monocyclic or multicyclic (e.g., bicyclic, tricyclic, etc.) hydrocarbon radical containing from 3 to 12 carbon atoms that is completely saturated or has one or more unsaturated bonds, and for the avoidance of doubt, the degree of unsaturation does not result in an aromatic ring system (e.g., phenyl).

Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “cyano” is a term of art and as used herein refers to —CN.

The term “halo” is a term of art and as used herein refers to —F, —Cl, —Br, or —I.

The term “haloalkyl” as used herein refers to an alkyl group, as defined herein, wherein some or all of the hydrogens are replaced with halogen atoms, as defined herein. Representative examples of haloalkyl include, but are not limited to, trifluoromethyl and fluoroethyl.

The term “hydroxy” is a term of art and as used herein refers to —OH.

The term “hydroxyalkyl”, as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “silyl”, as used herein, includes hydrocarbyl derivatives of the silyl (H₃Si—) group (i.e., (hydrocarbyl)₃Si—), wherein hydrocarbyl groups are univalent groups formed by removing a hydrogen atom from a hydrocarbon, e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy”, as used herein, means a silyl group, as defined herein, is appended to the parent molecule through an oxygen atom.

Certain compounds contained in compositions of the present invention may exist in particular geometric or stereoisomeric forms. In addition, compounds of the present invention may also be optically active. The present invention contemplates all such compounds, including cis- and trans-isomers, (R)- and (S)-enantiomers, diastereoisomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diasteromeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

In certain embodiments, the optional substituents contemplated in this invention include, for example, halogen, azide, alkyl, alkenyl, alkynyl, aryl, arylalkyl, heteroaryl, heteroarylalkyl, cycloalkyl, cycloalkenyl, cycloalkynyl, (cycloalkyl)alkyl, heterocycloalkyl, heterocycloalkenyl, heterocycloalkynyl, (heterocycloalkyl)alkyl, hydroxyl, alkoxy, alkenyloxy, alkynyloxy, amino, aminoalkyl, nitro, sulfhydryl, imino, amido (e.g., —C(O)NH (optionally substituted alkyl), —C(O)NH (optionally substituted cycloalkyl), and NHC(O)(optionally substituted alkyl)), phosphonate, phosphinate, carbonyl, carboxyl, carboxylalkyl (e.g., -alkylene-(COOH)), silyl, silyloxy, ether (e.g., -alkylene-O(alkyl)), alkylthio, sulfonyl (e.g., —S(O)₂alkyl), sulfonamido, Boc (—C(O)—O—C(CH₃)₃), ketone (e.g., —CO(alkyl)), aldehyde (—C(O)H), ester (e.g., -alkylene-COO(alkyl) or —COO(alkyl)), haloalkyl, hydroxyalkyl, alkoxyalkyl, haloalkoxy, haloalkoxyalkyl, and cyano.

The phrase “protecting group”, as used herein, means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts. P. G. M. Protective Groups in Organic Synthesis, 2^{nd} ed.; Wiley: New York, 1991). Protected forms of the inventive compounds are included within the scope of this invention.

For purposes of the invention, the chemical elements are identified in accordance with the Periodic Table of the Elements, CAS version. Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I, II, III, or IV. As another example, the salt may comprise less than one inorganic or organic acid molecule per molecule of base, such as two molecules of compound of Formula I, II, III, or IV per molecule of tartaric acid.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

The term “treat” as used herein means prevent, halt or slow the progression of, or eliminate a disease or condition in a subject. In one embodiment “treat” means halt or slow the progression of, or eliminate a disease or condition in a subject. In one embodiment, “treat” means reduce at least one objective manifestation of a disease or condition in a subject.

The term “effective amount” as used herein refers to an amount that is sufficient to bring about a desired biological effect.

The term “therapeutically effective amount” as used herein refers to an amount that is sufficient to bring about a desired therapeutic effect.

The term “inhibit” as used herein means decrease by an objectively measurable amount or extent. In various embodiments “inhibit” means decrease by at least 5, 10, 20, 30, 40, 50, 60, 70, 80, 90, or 95 percent compared to relevant control. In one embodiment “inhibit” means decrease 100 percent, i.e., halt or eliminate.

The term “subject” as used herein refers to a mammal. In various embodiments, a subject is a mouse, rat, rabbit, cat, dog, pig, sheep, horse, cow, or non-human primate. In one embodiment, a subject is a human.

### Compounds

The present invention provides a compound of Formula (I) or (II):

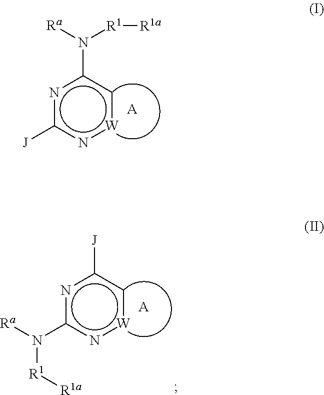

or a pharmaceutically acceptable salt thereof;
wherein:
A is a fused optionally substituted aromatic ring, heteroaromatic ring, cycloalkyl ring, cycloalkenyl ring, heterocycloalkyl ring, or heterocycloalkenyl ring;
W is C or N;
R^{a} represents H or alkyl;
R¹ represents heteroarylene;
R^{1a} represents H or optionally substituted —C(O)alkyl, —C(O)aryl, —C(O)heteroaryl, —C(O)O(alkyl), —C(O)(heterocyclyl), —C(O)NR^{x}R^{y}, alkyl, cycloalkyl, cycloalkenyl, heterocycloalkyl, aryl, or heteroaryl;
J represents optionally substituted alkyl, alkenyl, haloalkyl, hydroxyalkyl, alkoxyalkyl, aminoalkyl, cycloalkyl, (cycloalkyl)alkyl, heterocycloalkyl, (heterocycloalkyl)alkyl, or heterocycloalkenyl:
   further wherein when J is heterocycloalkyl or heterocycloalkenyl, the point of attachment in J to the rest of the compound is a carbon atom; and
R^{x} and R^{y} each independently represent H, alkyl, aralkyl, heteroaralkyl, aryl, heteroaryl, cycloalkyl, heterocycloalkyl, (cycloalkyl)alkyl, (heterocycloalkyl)alkyl, or hydroxyalkyl.

In certain embodiments, the compound of the invention is represented by formula (Ia) or formula (IIa):

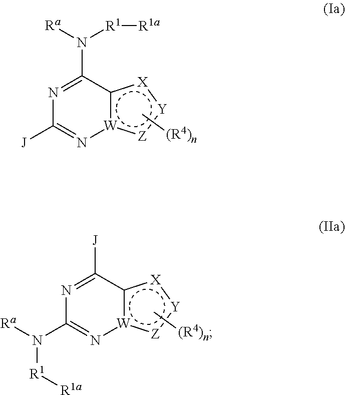

wherein:
W is C or N;
valence permitting, each of X, Y, and Z independently represent CH, CH₂, CO, N, NH, O, S, or SO₂, wherein any hydrogen of a CH, CH₂, or NH group is optionally replaced by an occurrence of R⁴;
R⁴, independently for each occurrence, represents halo, cyano, —CH₂C(O)NH₂, —C(O)R⁵, —C(O)OR⁵, —S(O)₂R⁵, or optionally substituted alkyl, alkenyl, haloalkyl, hydroxyalkyl, aminoalkyl, alkoxy, aryl, aralkyl, heteroaryl, heteroaralkyl, heterocycloalkyl, heterocycloalkenyl, (heterocycloalkyl)alkyl, cycloalkyl, (cycloalkyl)alkyl, halocycloalkyl, hydroxycycloalkyl, aminocycloalkyl, aryloxy, heteroaryloxy, arylalkyloxy, or heteroarylalkyloxy;
R⁵, independently for each occurrence, represents H or optionally substituted alkyl, aralkyl, aryl, heteroaralkyl, heteroaryl, cycloalkyl, heterocycloalkyl, (cycloalkyl)alkyl, or (heterocycloalkyl)alkyl; and
n is an integer from 0-4, as permitted by valence.

In certain embodiments, the compound of the invention is represented by formula (Ib) or formula (IIb):

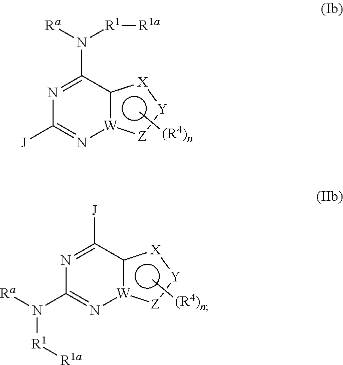

wherein each of X, Y, and Z independently represent CH, N, NH, O, S, or S02.

In certain embodiments, the compound of the invention is represented by formula (Ic) or (IIc):

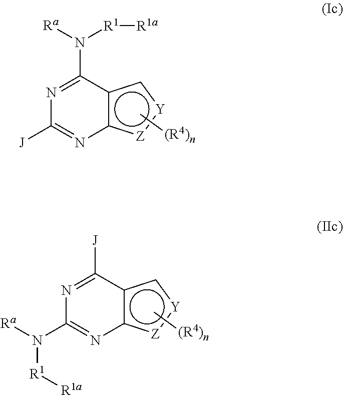

wherein each of Y and Z are independently selected from the group consisting of O, N, NH, and S.

In certain such embodiments, Y is N and Z is NH.

Alternatively, in certain embodiments, the compound of the invention is represented by formula (Id) or (IId):

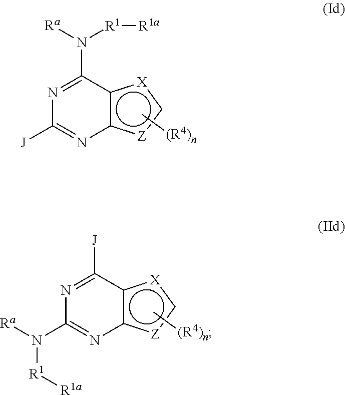

wherein at least one of X and Z is selected from the group consisting of O, N, NH, and S.

In certain embodiments of the compounds of formula (Id) and (IId), one of X and Z is selected from the group consisting of O, NH, and S; and the other of X and Z is CH. For example, X may be selected from the group consisting of O, NH, and S. Alternatively. Z may be selected from the group consisting of O, NH, and S. In further embodiments, one of X and Z is NH; and the other of X and Z is CH. In alternative embodiments, one of X and Z is O; and the other of X and Z is CH. In further alternative embodiments, one of X and Z is S; and the other of X and Z is CH.

In other embodiments of the compounds of formula (Id) and (IId), each of X and Z are selected from the group consisting of O, N, NH, and S. For example, one of X and Z may be N and the other of X and Z may be NH. Alternatively, one of X and Z may be S; and the other of X and Z may be N.

In certain embodiments, the compound of the invention is represented by formula (Ie) or (IIe):

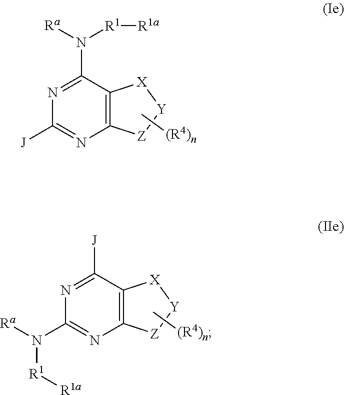

wherein X, Y, and Z independently represent CH₂, CO, NH, O, S, or SO₂.

In certain embodiments of the compounds of formula (Ie) or (IIe), each of X, Y, and Z is CH₂. In alternative embodiments, one of X, Y, and Z is O.

In any one of formulae (Ia), (IIa), (Ib), (IIb), (Ic), (IIc), (Id), (IId), (Ie), and (IIe), in certain embodiments, n is 0 or 1.

In certain embodiments, the compound of the invention is represented by formula (If) or formula (IIf):

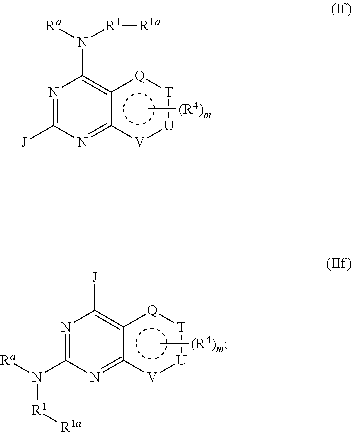

wherein:
valence permitting, each of Q, T, U, and V independently represent CH, CH₂, N, NH, O, or SO₂, wherein any hydrogen of a CH, CH₂, or NH group is optionally replaced by an occurrence of R⁴;
R⁴, independently for each occurrence, represents halo, cyano, —CH₂C(O)NH₂, —C(O)R⁵, —C(O)OR⁵, —S(O)₂R⁵, or optionally substituted alkyl, alkenyl, haloalkyl, hydroxyalkyl, aminoalkyl, alkoxy, aryl, aralkyl, heteroaryl, heteroaralkyl, heterocycloalkyl, heterocycloalkenyl, (heterocycloalkyl)alkyl, cycloalkyl, (cycloalkyl)alkyl, halocycloalkyl, hydroxycycloalkyl, aminocycloalkyl, aryloxy, heteroaryloxy, arylalkyloxy, or heteroarylalkyloxy;
R⁵, independently for each occurrence, represents H or optionally substituted alkyl, aralkyl, aryl, heteroaralkyl, heteroaryl, cycloalkyl, heterocycloalkyl, (cycloalkyl)alkyl, or (heterocycloalkyl)alkyl; and
   m is an integer from 0-4, as permitted by valence.
In further embodiments, the compound of the invention is represented by formula (Ig) or (IIg):

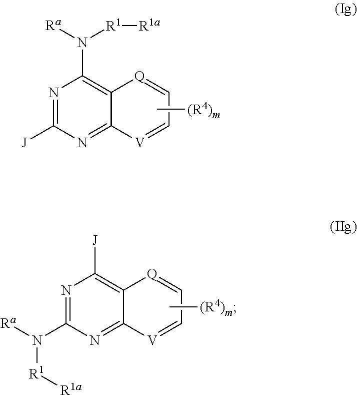

wherein Q represents CH or N; and V represents CH or N.

In certain such embodiments, Q is N; and V is CH. In other such embodiments, Q is CH; and V is N.

In other embodiments, the compound of the invention is represented by formula (Ih) or (IIh):

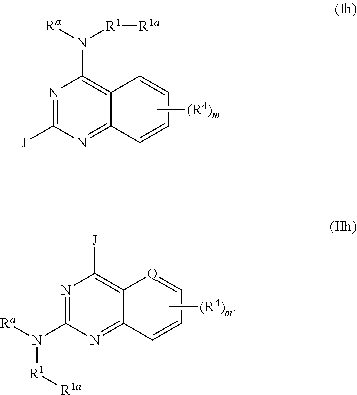

Alternatively, the compound of the invention may be represented by formula (Ij) or (IIj):

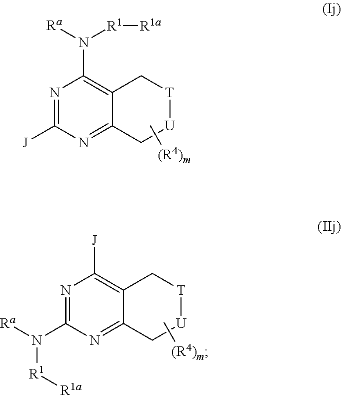

wherein T represents CH₂, NH, O, or SO₂, and U represents CH₂, NH, O, or SO₂.

In certain such embodiments, the compound is represented by formula (Ik) or (IIk):

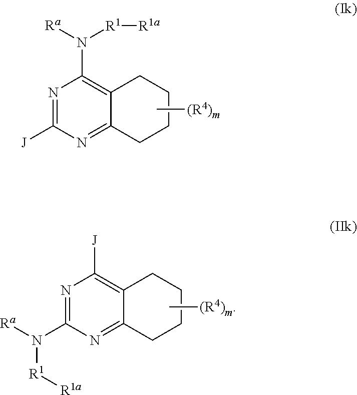

In other embodiments of the compound of formula (Ij) or (IIj), T is NH; and U is CH₂. Alternatively, T may be CH₂; and U may be NH.

In any one of formulae (If), (IIf), (Ig), (IIg), (Ih), (IIh), (Ij), (IIj), (Ik), and (IIk), in certain embodiments, m is 0 or 1.

In any of the foregoing embodiments, R⁴, if present, is halo, —C(O)O(alkyl), or is selected from the group consisting of optionally substituted alkyl, alkoxy, aryl, heterocycloalkyl, cycloalkyl, and (cycloalkyl)alkyl.

In any of the foregoing embodiments, R⁴, if present, is optionally substituted alkyl, cycloalkyl or alkoxy. In certain such embodiments, R⁴, if present, is optionally substituted alkyl or alkoxy.

In any of the foregoing embodiments, R^{a} may be H.

In any of the foregoing embodiments, R¹ is a nitrogen-containing heteroarylene, such as a 5-membered nitrogen-containing heteroarylene. In any of the foregoing embodiments, R¹ is imidazolene.

In any of the foregoing embodiments, —R¹—R^{1a} represents

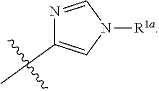

In any of the foregoing embodiments, R^{1a} represents H or optionally substituted —C(O)alkyl, —C(O)aryl, —C(O)heteroaryl, —C(O)O(alkyl), —C(O)(heterocyclyl), —C(O)NR^{x}R^{y}, alkyl, cycloalkyl, heterocycloalkyl, aryl, or heteroaryl.

In any of the foregoing embodiments, R^{1a} is optionally substituted phenyl. In any of the foregoing embodiments, R^{1a} may be phenyl, substituted by one or more occurrences of alkoxy. Preferably, R^{1a} is 3,4,5-trimethoxyphenyl.

In any of the foregoing embodiments. J is optionally substituted alkyl, alkenyl, cycloalkyl, or (cycloalkyl)alkyl. In any of the foregoing embodiments. J is optionally substituted alkyl or alkenyl. In further embodiments, J represents optionally substituted branched alkyl or alkenyl. For example, J may be isopropyl or isopropenyl.

In alternative embodiments, J represents optionally substituted cycloalkyl or (cycloalkyl)alkyl. In further alternative embodiments, J represents optionally substituted cycloalkyl.

In further alternative embodiments, J represents optionally substituted heterocycloalkyl.

In any of the foregoing embodiments, R^{1a} is phenyl, substituted by two or more occurrences of alkoxy, J is optionally substituted alkyl, alkenyl, cycloalkyl, or (cycloalkyl)alkyl, and R⁴, if present, is halo, —C(O)O(alkyl), or is selected from the group consisting of optionally substituted alkyl, alkoxy, aryl, heterocycloalkyl, cycloalkyl, and (cycloalkyl)alkyl.

In any of the foregoing embodiments. R^{1a} is phenyl, substituted by two or more occurrences of alkoxy (including 3,4,5-trimethoxyphenyl), J is optionally substituted alkyl, alkenyl, cycloalkyl, or (cycloalkyl)alkyl, and R⁴, if present, is selected from the group consisting of optionally substituted alkyl, cycloalkyl, or alkoxy.

In any of the foregoing embodiments, R^{1a} is 3,4,5-trimethoxyphenyl, J is optionally substituted alkyl, alkenyl, cycloalkyl, or (cycloalkyl)alkyl, and R⁴, if present, is halo, —C(O)O(alkyl), or is selected from the group consisting of optionally substituted alkyl, alkoxy, aryl, heterocycloalkyl, cycloalkyl, and (cycloalkyl)alkyl.

In any of the foregoing embodiments. R^{1a} is 3,4,5-trimethoxyphenyl. J is optionally substituted alkyl, alkenyl, cycloalkyl, or (cycloalkyl)alkyl, and R⁴, if present, is selected from the group consisting of optionally substituted alkyl, cycloalkyl, or alkoxy.

In certain embodiments, the compound of the invention is selected from the group consisting of the compounds depicted in the following table, or a pharmaceutically acceptable salt thereof:

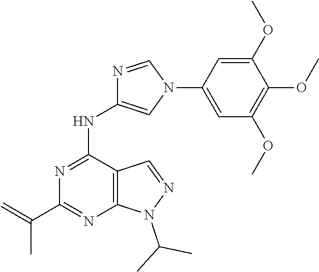 [SMILES: C=C(C)c1nc(Nc2cn(-c3cc(OC)c(OC)c(OC)c3)cn2)c2cnn(C(C)C)c2n1]

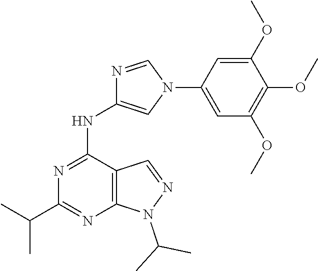 [SMILES: COc1cc(-n2cnc(Nc3nc(C(C)C)nc4c3cnn4C(C)C)c2)cc(OC)c1OC]

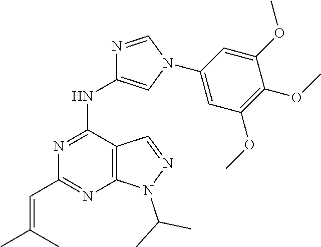 [SMILES: COc1cc(-n2cnc(Nc3nc(C=C(C)C)nc4c3cnn4C(C)C)c2)cc(OC)c1OC]

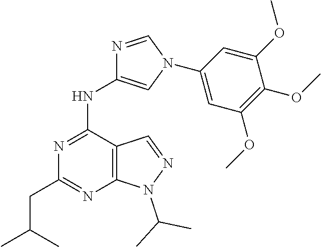 [SMILES: COc1cc(-n2cnc(Nc3nc(CC(C)C)nc4c3cnn4C(C)C)c2)cc(OC)c1OC]

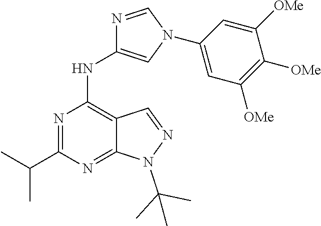 [SMILES: COc1cc(-n2cnc(Nc3nc(C(C)C)nc4c3cnn4C(C)(C)C)c2)cc(OC)c1OC]

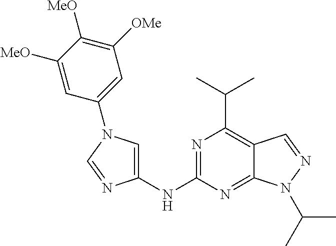 [SMILES: COc1cc(-n2cnc(Nc3nc(C(C)C)c4cnn(C(C)C)c4n3)c2)cc(OC)c1OC]

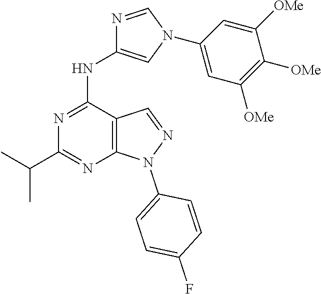 [SMILES: COc1cc(-n2cnc(Nc3nc(C(C)C)nc4c3cnn4-c3ccc(F)cc3)c2)cc(OC)c1OC]

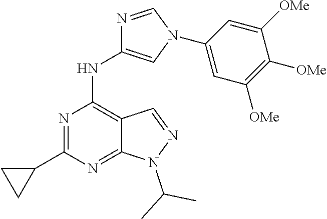 [SMILES: COc1cc(-n2cnc(Nc3nc(C4CC4)nc4c3cnn4C(C)C)c2)cc(OC)c1OC]

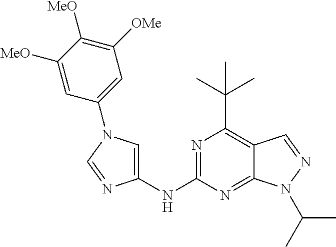 [SMILES: COc1cc(-n2cnc(Nc3nc(C(C)(C)C)c4cnn(C(C)C)c4n3)c2)cc(OC)c1OC]

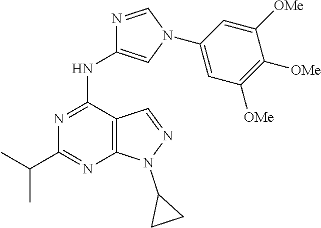 [SMILES: COc1cc(-n2cnc(Nc3nc(C(C)C)nc4c3cnn4C3CC3)c2)cc(OC)c1OC]

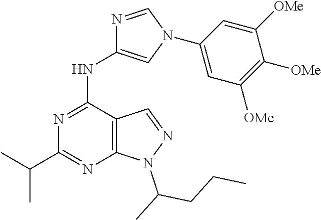 [SMILES: CCCC(C)n1ncc2c(Nc3cn(-c4cc(OC)c(OC)c(OC)c4)cn3)nc(C(C)C)nc21]

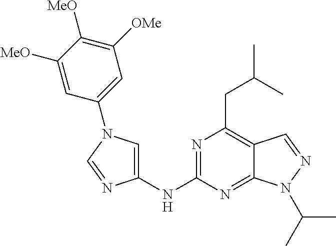 [SMILES: COc1cc(-n2cnc(Nc3nc(CC(C)C)c4cnn(C(C)C)c4n3)c2)cc(OC)c1OC]

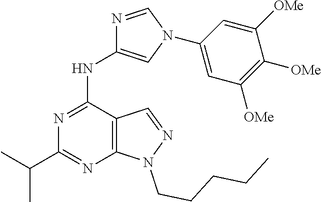 [SMILES: CCCCCn1ncc2c(Nc3cn(-c4cc(OC)c(OC)c(OC)c4)cn3)nc(C(C)C)nc21]

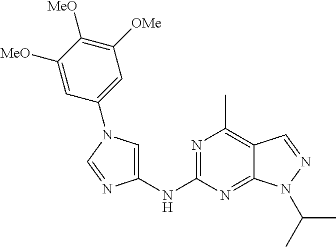 [SMILES: COc1cc(-n2cnc(Nc3nc(C)c4cnn(C(C)C)c4n3)c2)cc(OC)c1OC]

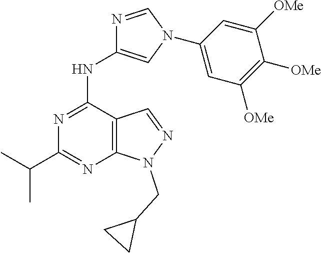 [SMILES: COc1cc(-n2cnc(Nc3nc(C(C)C)nc4c3cnn4CC3CC3)c2)cc(OC)c1OC]

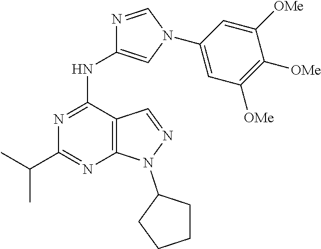 [SMILES: COc1cc(-n2cnc(Nc3nc(C(C)C)nc4c3cnn4C3CCCC3)c2)cc(OC)c1OC]

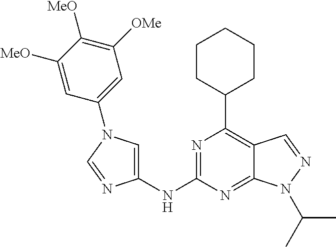 [SMILES: COc1cc(-n2cnc(Nc3nc(C4CCCCC4)c4cnn(C(C)C)c4n3)c2)cc(OC)c1OC]

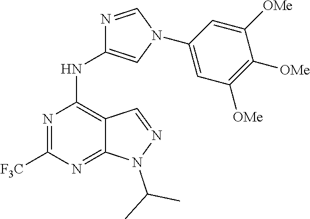 [SMILES: COc1cc(-n2cnc(Nc3nc(C(F)(F)F)nc4c3cnn4C(C)C)c2)cc(OC)c1OC]

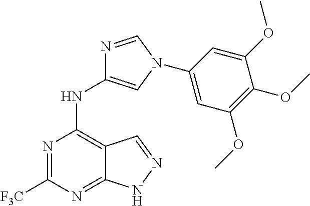 [SMILES: COc1cc(-n2cnc(Nc3nc(C(F)(F)F)nc4[nH]ncc34)c2)cc(OC)c1OC]

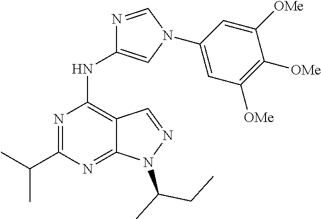 [SMILES: CC[C@@H](C)n1ncc2c(Nc3cn(-c4cc(OC)c(OC)c(OC)c4)cn3)nc(C(C)C)nc21]

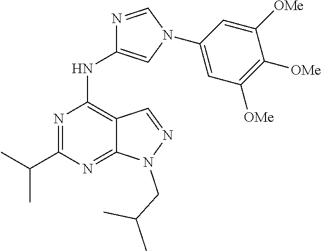 [SMILES: COc1cc(-n2cnc(Nc3nc(C(C)C)nc4c3cnn4CC(C)C)c2)cc(OC)c1OC]

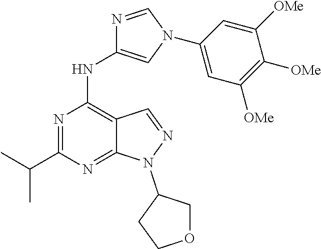 [SMILES: COc1cc(-n2cnc(Nc3nc(C(C)C)nc4c3cnn4C3CCOC3)c2)cc(OC)c1OC]

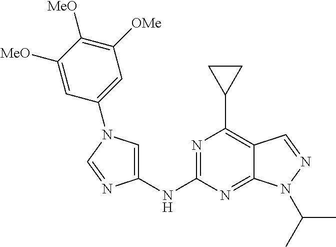 [SMILES: COc1cc(-n2cnc(Nc3nc(C4CC4)c4cnn(C(C)C)c4n3)c2)cc(OC)c1OC]

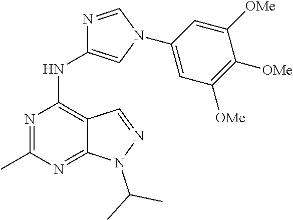 [SMILES: COc1cc(-n2cnc(Nc3nc(C)nc4c3cnn4C(C)C)c2)cc(OC)c1OC]

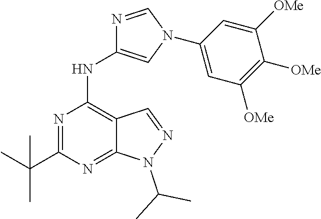 [SMILES: COc1cc(-n2cnc(Nc3nc(C(C)(C)C)nc4c3cnn4C(C)C)c2)cc(OC)c1OC]

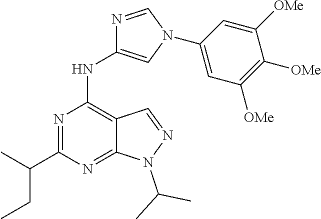 [SMILES: CCC(C)c1nc(Nc2cn(-c3cc(OC)c(OC)c(OC)c3)cn2)c2cnn(C(C)C)c2n1]

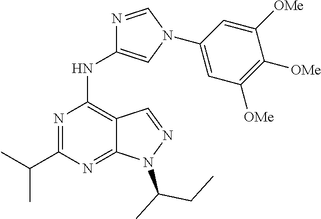 [SMILES: CC[C@@H](C)n1ncc2c(Nc3cn(-c4cc(OC)c(OC)c(OC)c4)cn3)nc(C(C)C)nc21]

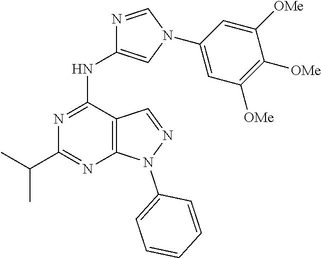 [SMILES: COc1cc(-n2cnc(Nc3nc(C(C)C)nc4c3cnn4-c3ccccc3)c2)cc(OC)c1OC]

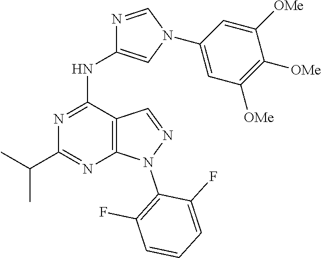 [SMILES: COc1cc(-n2cnc(Nc3nc(C(C)C)nc4c3cnn4-c3c(F)cccc3F)c2)cc(OC)c1OC]

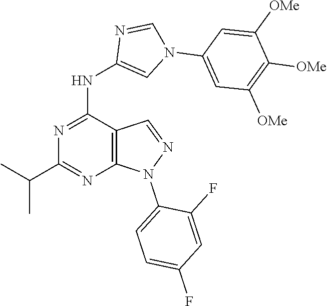 [SMILES: COc1cc(-n2cnc(Nc3nc(C(C)C)nc4c3cnn4-c3ccc(F)cc3F)c2)cc(OC)c1OC]

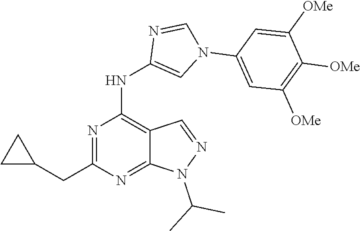 [SMILES: COc1cc(-n2cnc(Nc3nc(CC4CC4)nc4c3cnn4C(C)C)c2)cc(OC)c1OC]

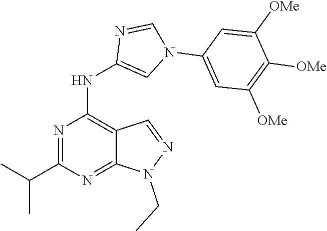 [SMILES: CCn1ncc2c(Nc3cn(-c4cc(OC)c(OC)c(OC)c4)cn3)nc(C(C)C)nc21]

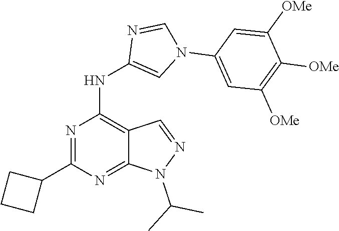 [SMILES: COc1cc(-n2cnc(Nc3nc(C4CCC4)nc4c3cnn4C(C)C)c2)cc(OC)c1OC]

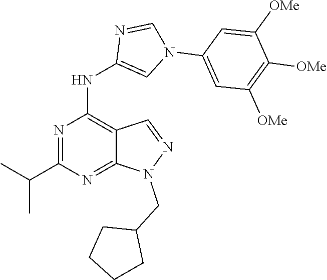 [SMILES: COc1cc(-n2cnc(Nc3nc(C(C)C)nc4c3cnn4CC3CCCC3)c2)cc(OC)c1OC]

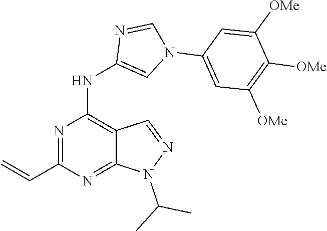 [SMILES: C=Cc1nc(Nc2cn(-c3cc(OC)c(OC)c(OC)c3)cn2)c2cnn(C(C)C)c2n1]

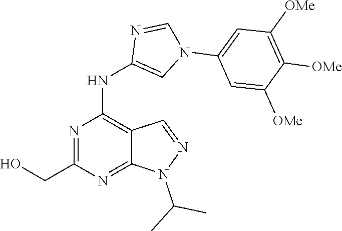 [SMILES: COc1cc(-n2cnc(Nc3nc(CO)nc4c3cnn4C(C)C)c2)cc(OC)c1OC]

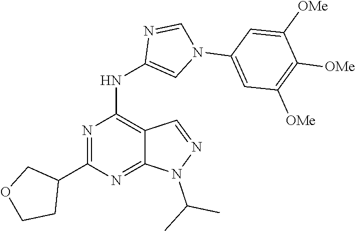 [SMILES: COc1cc(-n2cnc(Nc3nc(C4CCOC4)nc4c3cnn4C(C)C)c2)cc(OC)c1OC]

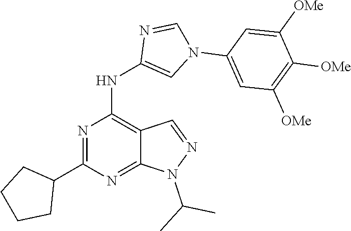 [SMILES: COc1cc(-n2cnc(Nc3nc(C4CCCC4)nc4c3cnn4C(C)C)c2)cc(OC)c1OC]

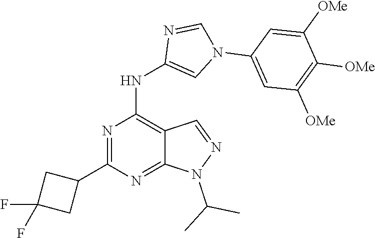 [SMILES: COc1cc(-n2cnc(Nc3nc(C4CC(F)(F)C4)nc4c3cnn4C(C)C)c2)cc(OC)c1OC]

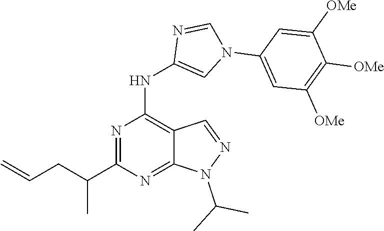 [SMILES: C=CCC(C)c1nc(Nc2cn(-c3cc(OC)c(OC)c(OC)c3)cn2)c2cnn(C(C)C)c2n1]

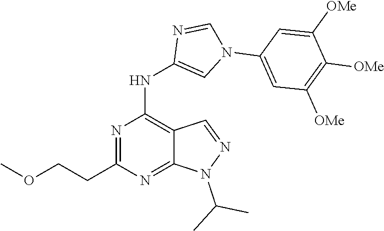 [SMILES: COCCc1nc(Nc2cn(-c3cc(OC)c(OC)c(OC)c3)cn2)c2cnn(C(C)C)c2n1]

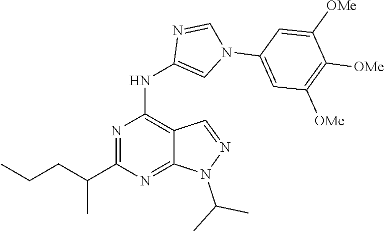 [SMILES: CCCC(C)c1nc(Nc2cn(-c3cc(OC)c(OC)c(OC)c3)cn2)c2cnn(C(C)C)c2n1]

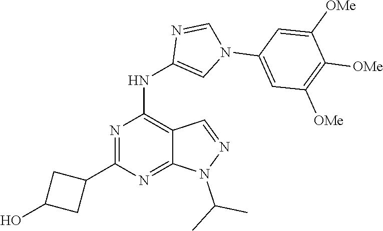 [SMILES: COc1cc(-n2cnc(Nc3nc(C4CC(O)C4)nc4c3cnn4C(C)C)c2)cc(OC)c1OC]

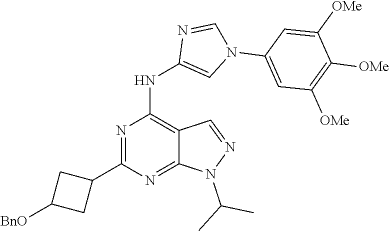 [SMILES: COc1cc(-n2cnc(Nc3nc(C4CC(OCc5ccccc5)C4)nc4c3cnn4C(C)C)c2)cc(OC)c1OC]

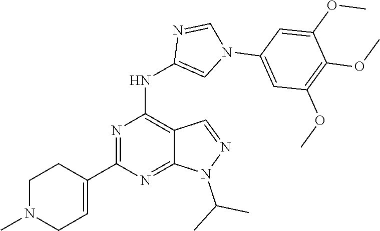

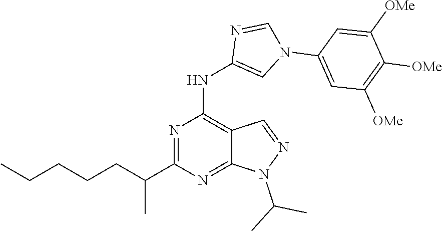 [SMILES: CCCCCC(C)c1nc(Nc2cn(-c3cc(OC)c(OC)c(OC)c3)cn2)c2cnn(C(C)C)c2n1]

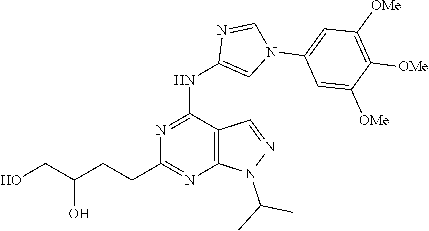 [SMILES: COc1cc(-n2cnc(Nc3nc(CCC(O)CO)nc4c3cnn4C(C)C)c2)cc(OC)c1OC]

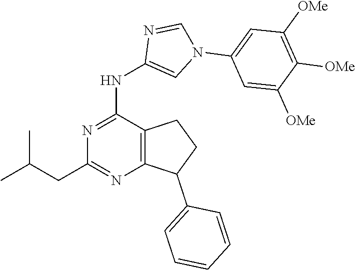 [SMILES: COc1cc(-n2cnc(Nc3nc(CC(C)C)nc4c3CCC4c3ccccc3)c2)cc(OC)c1OC]

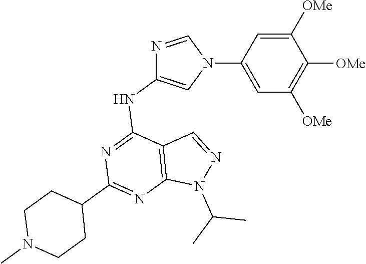 [SMILES: COc1cc(-n2cnc(Nc3nc(C4CCN(C)CC4)nc4c3cnn4C(C)C)c2)cc(OC)c1OC]

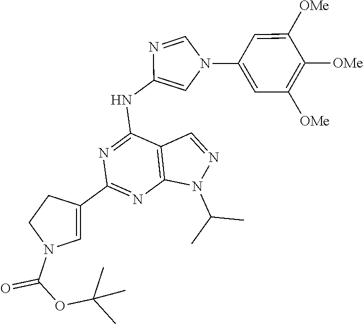 [SMILES: COc1cc(-n2cnc(Nc3nc(C4=CN(C(=O)OC(C)(C)C)CC4)nc4c3cnn4C(C)C)c2)cc(OC)c1OC]

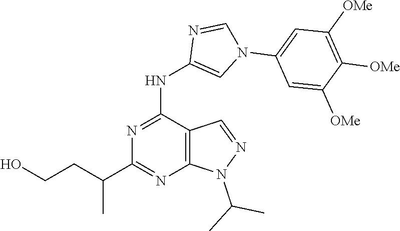 [SMILES: COc1cc(-n2cnc(Nc3nc(C(C)CCO)nc4c3cnn4C(C)C)c2)cc(OC)c1OC]

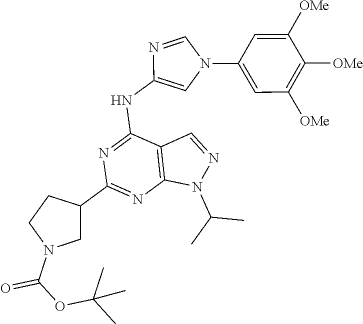 [SMILES: COc1cc(-n2cnc(Nc3nc(C4CCN(C(=O)OC(C)(C)C)C4)nc4c3cnn4C(C)C)c2)cc(OC)c1OC]

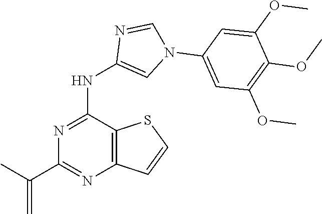 [SMILES: C=C(C)c1nc(Nc2cn(-c3cc(OC)c(OC)c(OC)c3)cn2)c2sccc2n1]

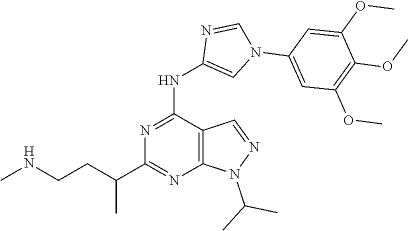

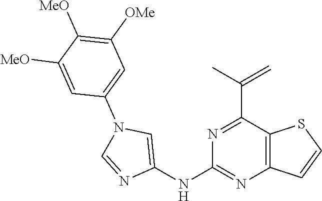 [SMILES: C=C(C)c1nc(Nc2cn(-c3cc(OC)c(OC)c(OC)c3)cn2)nc2ccsc12]

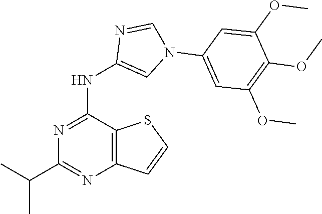 [SMILES: COc1cc(-n2cnc(Nc3nc(C(C)C)nc4ccsc34)c2)cc(OC)c1OC]

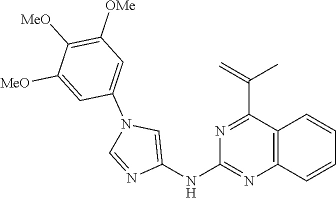 [SMILES: C=C(C)c1nc(Nc2cn(-c3cc(OC)c(OC)c(OC)c3)cn2)nc2ccccc12]

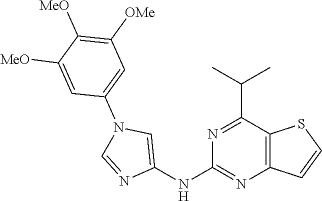 [SMILES: COc1cc(-n2cnc(Nc3nc(C(C)C)c4sccc4n3)c2)cc(OC)c1OC]

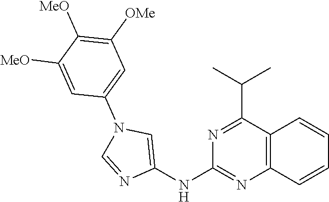 [SMILES: COc1cc(-n2cnc(Nc3nc(C(C)C)c4ccccc4n3)c2)cc(OC)c1OC]

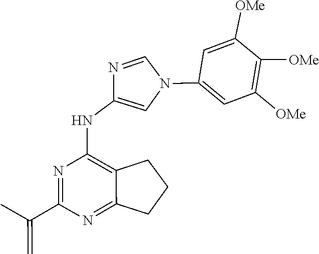 [SMILES: C=C(C)c1nc2c(c(Nc3cn(-c4cc(OC)c(OC)c(OC)c4)cn3)n1)CCC2]

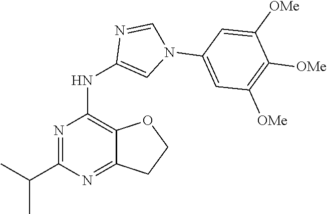 [SMILES: COc1cc(-n2cnc(Nc3nc(C(C)C)nc4c3OCC4)c2)cc(OC)c1OC]

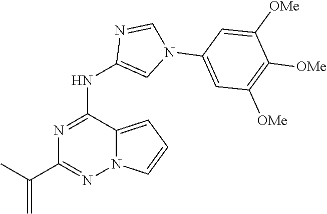 [SMILES: C=C(C)c1nc(Nc2cn(-c3cc(OC)c(OC)c(OC)c3)cn2)c2cccn2n1]

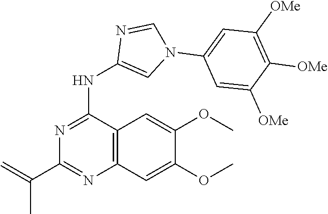 [SMILES: C=C(C)c1nc(Nc2cn(-c3cc(OC)c(OC)c(OC)c3)cn2)c2cc(OC)c(OC)cc2n1]

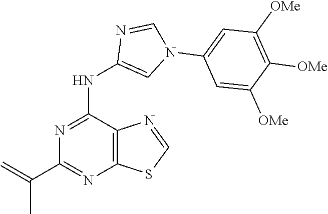 [SMILES: C=C(C)c1nc(Nc2cn(-c3cc(OC)c(OC)c(OC)c3)cn2)c2ncsc2n1]

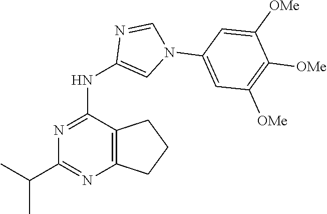 [SMILES: COc1cc(-n2cnc(Nc3nc(C(C)C)nc4c3CCC4)c2)cc(OC)c1OC]

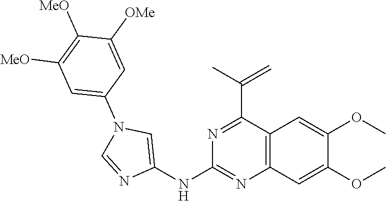 [SMILES: C=C(C)c1nc(Nc2cn(-c3cc(OC)c(OC)c(OC)c3)cn2)nc2cc(OC)c(OC)cc12]

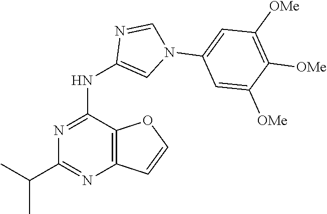 [SMILES: COc1cc(-n2cnc(Nc3nc(C(C)C)nc4ccoc34)c2)cc(OC)c1OC]

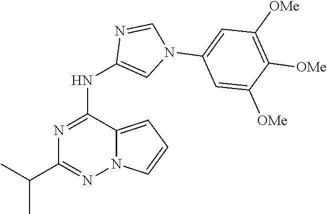 [SMILES: COc1cc(-n2cnc(Nc3nc(C(C)C)nn4cccc34)c2)cc(OC)c1OC]

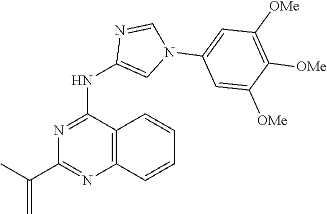 [SMILES: C=C(C)c1nc(Nc2cn(-c3cc(OC)c(OC)c(OC)c3)cn2)c2ccccc2n1]

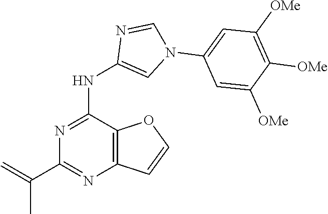 [SMILES: C=C(C)c1nc(Nc2cn(-c3cc(OC)c(OC)c(OC)c3)cn2)c2occc2n1]

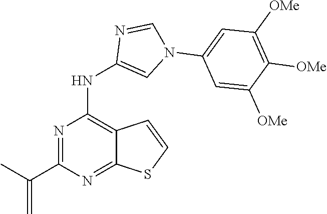 [SMILES: C=C(C)c1nc(Nc2cn(-c3cc(OC)c(OC)c(OC)c3)cn2)c2ccsc2n1]

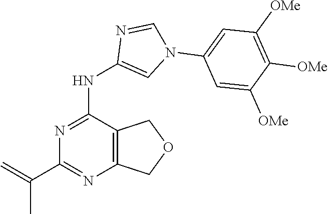 [SMILES: C=C(C)c1nc2c(c(Nc3cn(-c4cc(OC)c(OC)c(OC)c4)cn3)n1)COC2]

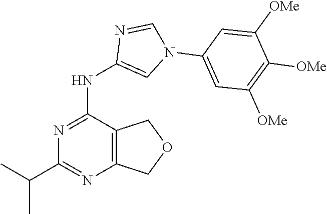 [SMILES: COc1cc(-n2cnc(Nc3nc(C(C)C)nc4c3COC4)c2)cc(OC)c1OC]

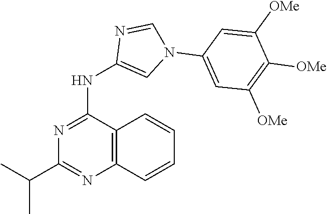 [SMILES: COc1cc(-n2cnc(Nc3nc(C(C)C)nc4ccccc34)c2)cc(OC)c1OC]

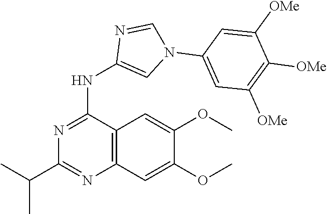 [SMILES: COc1cc2nc(C(C)C)nc(Nc3cn(-c4cc(OC)c(OC)c(OC)c4)cn3)c2cc1OC]

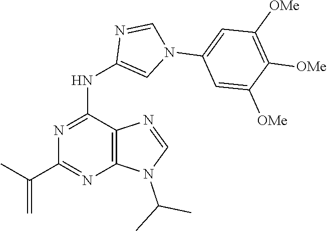 [SMILES: C=C(C)c1nc(Nc2cn(-c3cc(OC)c(OC)c(OC)c3)cn2)c2ncn(C(C)C)c2n1]

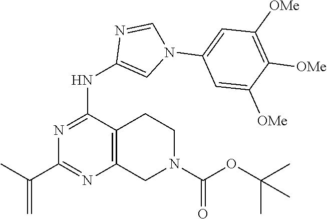 [SMILES: C=C(C)c1nc2c(c(Nc3cn(-c4cc(OC)c(OC)c(OC)c4)cn3)n1)CCN(C(=O)OC(C)(C)C)C2]

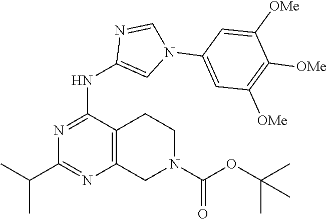 [SMILES: COc1cc(-n2cnc(Nc3nc(C(C)C)nc4c3CCN(C(=O)OC(C)(C)C)C4)c2)cc(OC)c1OC]

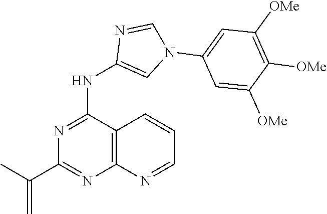 [SMILES: C=C(C)c1nc(Nc2cn(-c3cc(OC)c(OC)c(OC)c3)cn2)c2cccnc2n1]

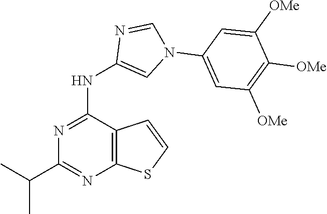 [SMILES: COc1cc(-n2cnc(Nc3nc(C(C)C)nc4sccc34)c2)cc(OC)c1OC]

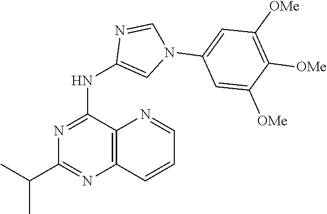 [SMILES: COc1cc(-n2cnc(Nc3nc(C(C)C)nc4cccnc34)c2)cc(OC)c1OC]

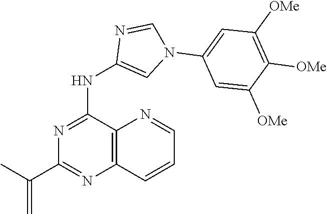 [SMILES: C=C(C)c1nc(Nc2cn(-c3cc(OC)c(OC)c(OC)c3)cn2)c2ncccc2n1]

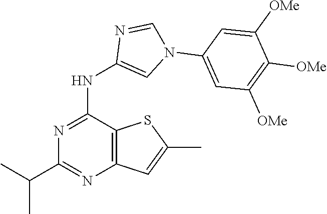 [SMILES: COc1cc(-n2cnc(Nc3nc(C(C)C)nc4cc(C)sc34)c2)cc(OC)c1OC]

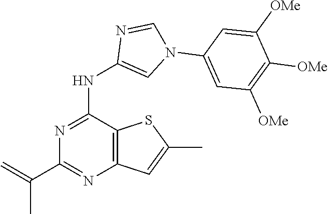 [SMILES: C=C(C)c1nc(Nc2cn(-c3cc(OC)c(OC)c(OC)c3)cn2)c2sc(C)cc2n1]

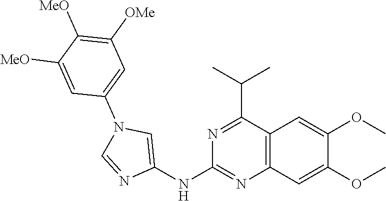 [SMILES: COc1cc2nc(Nc3cn(-c4cc(OC)c(OC)c(OC)c4)cn3)nc(C(C)C)c2cc1OC]

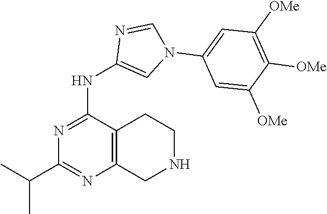 [SMILES: COc1cc(-n2cnc(Nc3nc(C(C)C)nc4c3CCNC4)c2)cc(OC)c1OC]

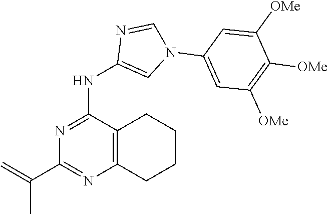 [SMILES: C=C(C)c1nc2c(c(Nc3cn(-c4cc(OC)c(OC)c(OC)c4)cn3)n1)CCCC2]

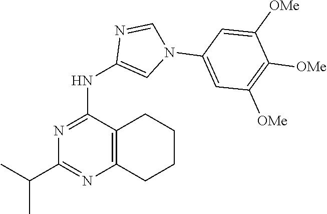 [SMILES: COc1cc(-n2cnc(Nc3nc(C(C)C)nc4c3CCCC4)c2)cc(OC)c1OC]

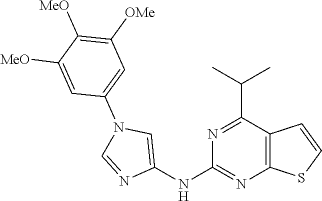 [SMILES: COc1cc(-n2cnc(Nc3nc(C(C)C)c4ccsc4n3)c2)cc(OC)c1OC]

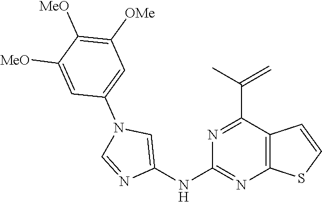 [SMILES: C=C(C)c1nc(Nc2cn(-c3cc(OC)c(OC)c(OC)c3)cn2)nc2sccc12]

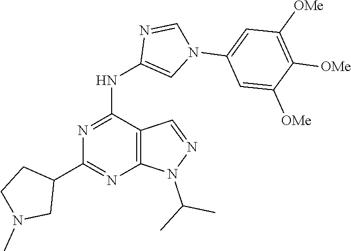 [SMILES: COc1cc(-n2cnc(Nc3nc(C4CCN(C)C4)nc4c3cnn4C(C)C)c2)cc(OC)c1OC]

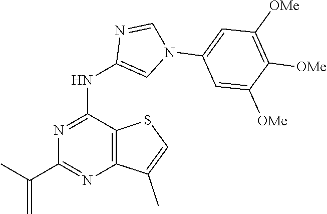 [SMILES: C=C(C)c1nc(Nc2cn(-c3cc(OC)c(OC)c(OC)c3)cn2)c2scc(C)c2n1]

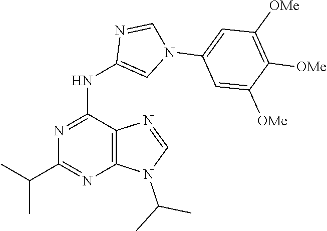 [SMILES: COc1cc(-n2cnc(Nc3nc(C(C)C)nc4c3ncn4C(C)C)c2)cc(OC)c1OC]

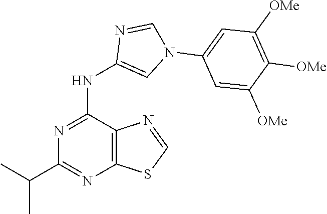 [SMILES: COc1cc(-n2cnc(Nc3nc(C(C)C)nc4scnc34)c2)cc(OC)c1OC]

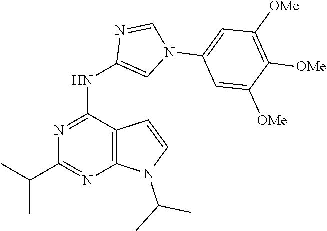 [SMILES: COc1cc(-n2cnc(Nc3nc(C(C)C)nc4c3ccn4C(C)C)c2)cc(OC)c1OC]

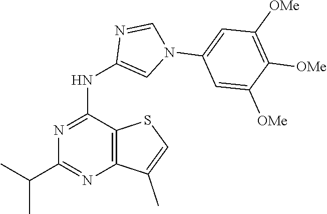 [SMILES: COc1cc(-n2cnc(Nc3nc(C(C)C)nc4c(C)csc34)c2)cc(OC)c1OC]

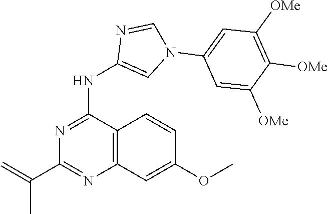 [SMILES: C=C(C)c1nc(Nc2cn(-c3cc(OC)c(OC)c(OC)c3)cn2)c2ccc(OC)cc2n1]

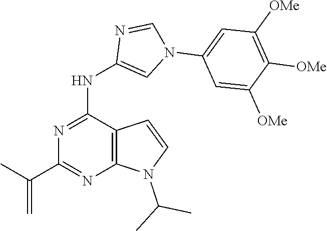 [SMILES: C=C(C)c1nc(Nc2cn(-c3cc(OC)c(OC)c(OC)c3)cn2)c2ccn(C(C)C)c2n1]

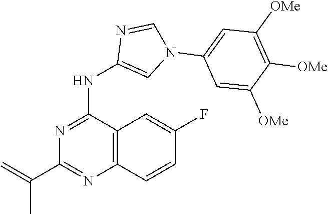 [SMILES: C=C(C)c1nc(Nc2cn(-c3cc(OC)c(OC)c(OC)c3)cn2)c2cc(F)ccc2n1]

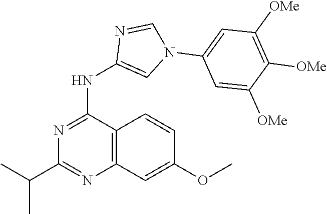 [SMILES: COc1ccc2c(Nc3cn(-c4cc(OC)c(OC)c(OC)c4)cn3)nc(C(C)C)nc2c1]

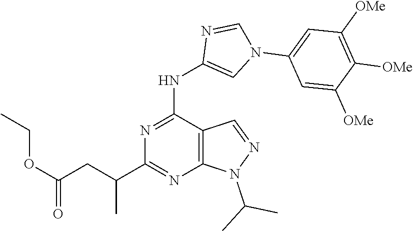 [SMILES: CCOC(=O)CC(C)c1nc(Nc2cn(-c3cc(OC)c(OC)c(OC)c3)cn2)c2cnn(C(C)C)c2n1]

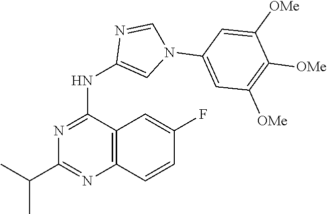 [SMILES: COc1cc(-n2cnc(Nc3nc(C(C)C)nc4ccc(F)cc34)c2)cc(OC)c1OC]

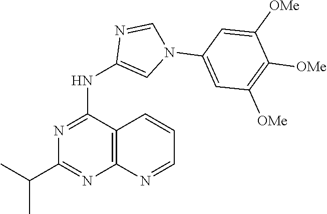 [SMILES: COc1cc(-n2cnc(Nc3nc(C(C)C)nc4ncccc34)c2)cc(OC)c1OC]

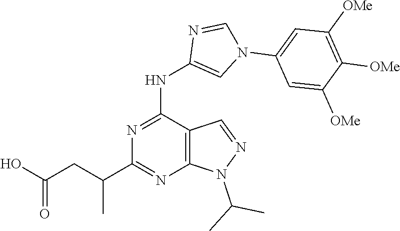 [SMILES: COc1cc(-n2cnc(Nc3nc(C(C)CC(=O)O)nc4c3cnn4C(C)C)c2)cc(OC)c1OC]

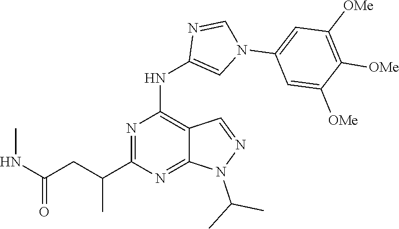 [SMILES: CNC(=O)CC(C)c1nc(Nc2cn(-c3cc(OC)c(OC)c(OC)c3)cn2)c2cnn(C(C)C)c2n1]

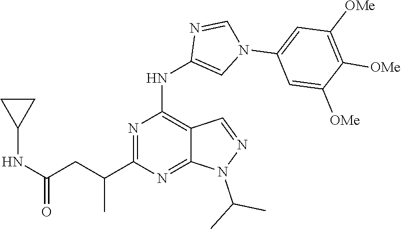 [SMILES: COc1cc(-n2cnc(Nc3nc(C(C)CC(=O)NC4CC4)nc4c3cnn4C(C)C)c2)cc(OC)c1OC]

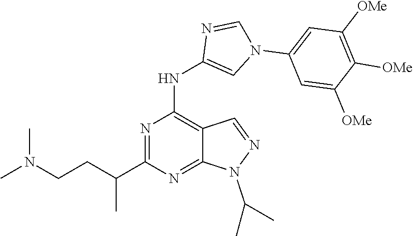 [SMILES: COc1cc(-n2cnc(Nc3nc(C(C)CCN(C)C)nc4c3cnn4C(C)C)c2)cc(OC)c1OC]

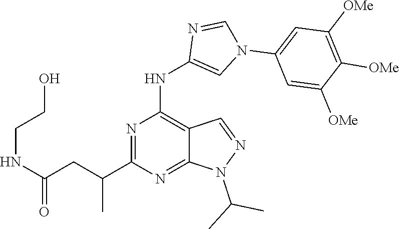 [SMILES: COc1cc(-n2cnc(Nc3nc(C(C)CC(=O)NCCO)nc4c3cnn4C(C)C)c2)cc(OC)c1OC]

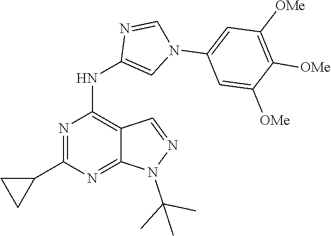 [SMILES: COc1cc(-n2cnc(Nc3nc(C4CC4)nc4c3cnn4C(C)(C)C)c2)cc(OC)c1OC]

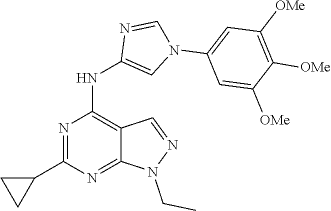 [SMILES: CCn1ncc2c(Nc3cn(-c4cc(OC)c(OC)c(OC)c4)cn3)nc(C3CC3)nc21]

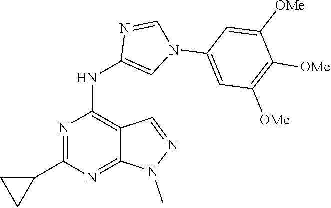 [SMILES: COc1cc(-n2cnc(Nc3nc(C4CC4)nc4c3cnn4C)c2)cc(OC)c1OC]

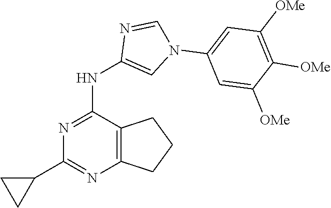 [SMILES: COc1cc(-n2cnc(Nc3nc(C4CC4)nc4c3CCC4)c2)cc(OC)c1OC]

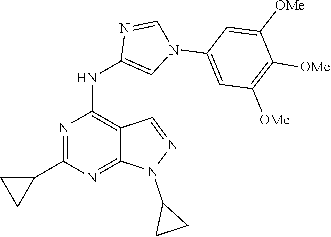 [SMILES: COc1cc(-n2cnc(Nc3nc(C4CC4)nc4c3cnn4C3CC3)c2)cc(OC)c1OC]

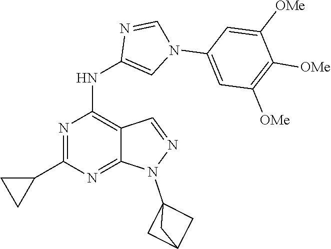 [SMILES: COc1cc(-n2cnc(Nc3nc(C4CC4)nc4c3cnn4C34CC(C3)C4)c2)cc(OC)c1OC]

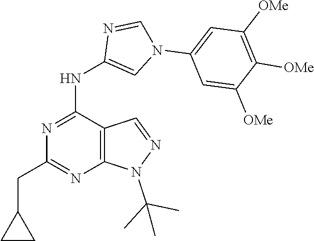 [SMILES: COc1cc(-n2cnc(Nc3nc(CC4CC4)nc4c3cnn4C(C)(C)C)c2)cc(OC)c1OC]

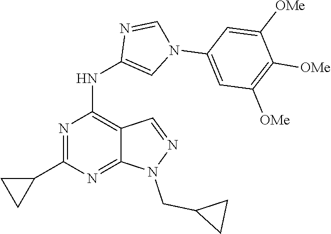 [SMILES: COc1cc(-n2cnc(Nc3nc(C4CC4)nc4c3cnn4CC3CC3)c2)cc(OC)c1OC]

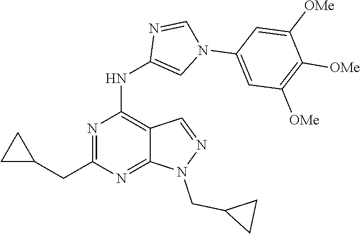 [SMILES: COc1cc(-n2cnc(Nc3nc(CC4CC4)nc4c3cnn4CC3CC3)c2)cc(OC)c1OC]

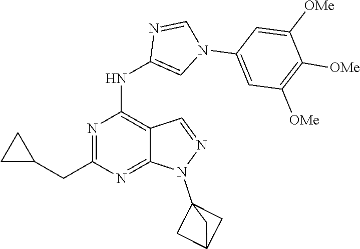 [SMILES: COc1cc(-n2cnc(Nc3nc(CC4CC4)nc4c3cnn4C34CC(C3)C4)c2)cc(OC)c1OC]

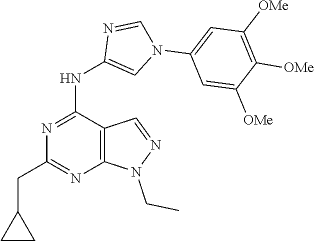 [SMILES: CCn1ncc2c(Nc3cn(-c4cc(OC)c(OC)c(OC)c4)cn3)nc(CC3CC3)nc21]

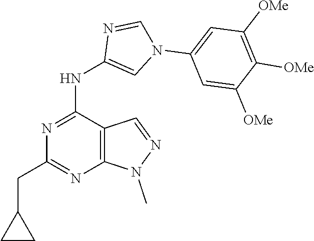 [SMILES: COc1cc(-n2cnc(Nc3nc(CC4CC4)nc4c3cnn4C)c2)cc(OC)c1OC]

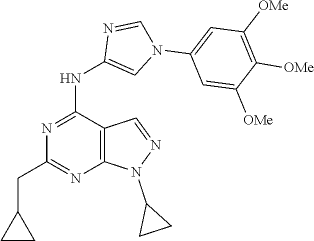 [SMILES: COc1cc(-n2cnc(Nc3nc(CC4CC4)nc4c3cnn4C3CC3)c2)cc(OC)c1OC]

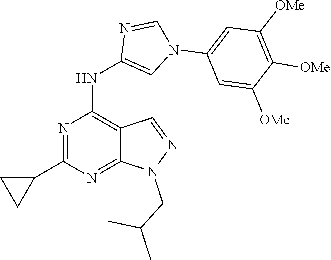 [SMILES: COc1cc(-n2cnc(Nc3nc(C4CC4)nc4c3cnn4CC(C)C)c2)cc(OC)c1OC]

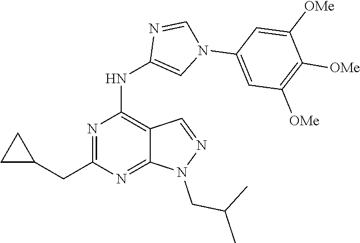 [SMILES: COc1cc(-n2cnc(Nc3nc(CC4CC4)nc4c3cnn4CC(C)C)c2)cc(OC)c1OC]

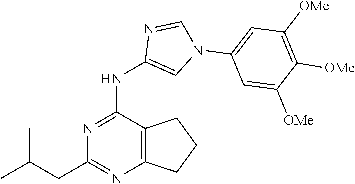 [SMILES: COc1cc(-n2cnc(Nc3nc(CC(C)C)nc4c3CCC4)c2)cc(OC)c1OC]

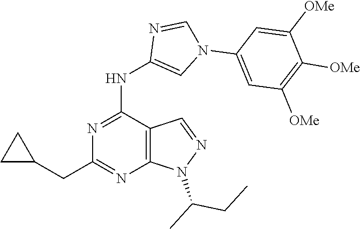 [SMILES: CC[C@H](C)n1ncc2c(Nc3cn(-c4cc(OC)c(OC)c(OC)c4)cn3)nc(CC3CC3)nc21]

In further aspects, the invention provides a compound represented by formula (III) or formula (IV):

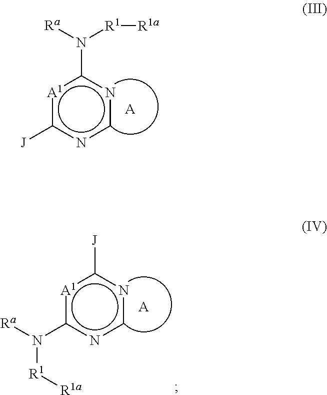

or a pharmaceutically acceptable salt thereof;
wherein:
A is a fused optionally substituted aromatic ring, heteroaromatic ring, cycloalkyl ring, cycloalkenyl ring, heterocycloalkyl ring, or heterocycloalkenyl ring;
A¹ is CH or N;
R^{a} represents H or alkyl;
R¹ represents heteroarylene;
R^{1a} represents H or optionally substituted —C(O)alkyl, —C(O)aryl, —C(O)heteroaryl, —C(O)O(alkyl), —C(O)(heterocyclyl), —C(O)NR^{x}R^{y}, alkyl, cycloalkyl, cycloalkenyl, heterocycloalkyl, aryl, or heteroaryl;
J represents optionally substituted alkyl, alkenyl, haloalkyl, hydroxyalkyl, alkoxyalkyl, cycloalkyl. (cycloalkyl)alkyl, heterocycloalkyl, (heterocycloalkyl)alkyl, or heterocycloalkenyl; and
R^{x} and R^{y} each independently represent H, alkyl, aralkyl, heteroaralkyl, aryl, heteroaryl, cycloalkyl, heterocycloalkyl, (cycloalkyl)alkyl, (heterocycloalkyl)alkyl, or hydroxyalkyl.

In certain embodiments, the compound is represented by formula (IIIa) or formula (IVa):

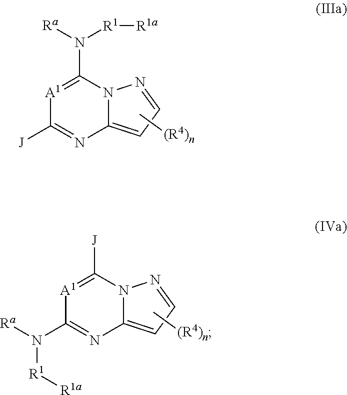

wherein:
R⁴, independently for each occurrence, represents halo, cyano, —CH₂C(O)NH₂, —C(O)R⁵, —C(O)OR⁵, —S(O)₂R⁵, or optionally substituted alkyl, alkenyl, haloalkyl, hydroxyalkyl, aminoalkyl, alkoxy, aryl, aralkyl, heteroaryl, heteroaralkyl, heterocycloalkyl, heterocycloalkenyl, (heterocycloalkyl)alkyl, cycloalkyl, (cycloalkyl)alkyl, halocycloalkyl, hydroxycycloalkyl, aminocycloalkyl, aryloxy, heteroaryloxy, arylalkyloxy, or heteroarylalkyloxy;
R⁵, independently for each occurrence, represents optionally substituted alkyl, aralkyl, aryl, heteroaralkyl, heteroaryl, cycloalkyl, heterocycloalkyl, (cycloalkyl)alkyl, or (heterocycloalkyl)alkyl; and
n is an integer from 0-2.

In certain embodiments, A¹ is CH. Alternatively, A¹ may be N.

In any one of formulae (IIIa) and (IVa), in certain embodiments, n is 0 or 1.

In any of the foregoing embodiments, R⁴, if present, is alkyl.

In any of the foregoing embodiments, R^{a} may be H.

In any of the foregoing embodiments, R¹ is a nitrogen-containing heteroarylene, such as a 5-membered nitrogen-containing heteroarylene. In any of the foregoing embodiments, R¹ is imidazolene.

In any of the foregoing embodiments, —R¹—R^{1a} represents

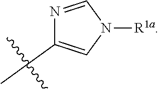

In any of the foregoing embodiments, R^{1a} may be optionally substituted phenyl. In any of the foregoing embodiments, R^{1a} may be phenyl, substituted by one or more occurrences of alkoxy. In any of the foregoing embodiments, R^{1a} is 3,4,5-trimethoxyphenyl.

In any of the foregoing embodiments, J is optionally substituted cycloalkyl.

In certain embodiments, the compound of the invention is selected from the group consisting of the compounds depicted in the following table:

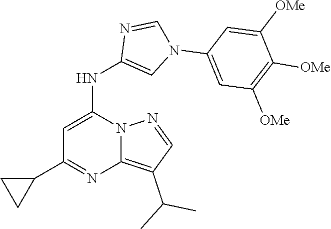 [SMILES: COc1cc(-n2cnc(Nc3cc(C4CC4)nc4c(C(C)C)cnn34)c2)cc(OC)c1OC]

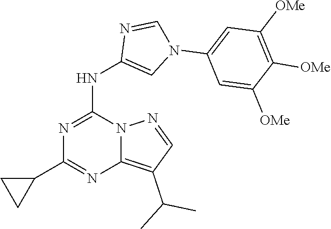 [SMILES: COc1cc(-n2cnc(Nc3nc(C4CC4)nc4c(C(C)C)cnn34)c2)cc(OC)c1OC]

### Pharmaceutical Compositions

The invention provides pharmaceutical compositions, each comprising one or more compounds of the invention, or pharmaceutically acceptable salts thereof, and a pharmaceutically acceptable carrier. In certain embodiments, the pharmaceutical composition comprises a compound of the invention and a pharmaceutically acceptable carrier. In certain embodiments, the pharmaceutical composition comprises a plurality of compounds of the invention, or pharmaceutically acceptable salts thereof, and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of a disease or condition that would be benefitted by inhibition of ALK2 kinase.

Pharmaceutical compositions of the invention can be prepared by combining one or more compounds of the invention, or pharmaceutically acceptable salts thereof, with a pharmaceutically acceptable carrier and, optionally, one or more additional pharmaceutically active agents.

### Methods of Use

The present invention provides compounds, and pharmaceutically acceptable salts thereof, that are useful for treating or preventing a disease or condition whose treatment would benefit from ALK2 kinase inhibition.

In certain aspects, the invention provides a method of inhibiting ALK2 kinase, comprising administering to a subject in need thereof an effective amount of a compound of the invention (e.g., a compound of formula (I), (II), (III), or (IV)), or a pharmaceutically acceptable salt thereof. In certain aspects, the invention provides a method of inhibiting ALK2 kinase, comprising administering to a subject in need thereof an amount of a compound of the invention (e.g., a compound of formula (I), (II), (III), or (IV)), or a pharmaceutically acceptable salt thereof.

In certain aspects, the invention provides a compound of the invention, or a pharmaceutically acceptable salt thereof, for use as a medicament.

In certain aspects, the invention provides methods of treating fibrodysplasia ossificans progressive, comprising the step of administering to a subject in need thereof a therapeutically effective amount of a compound of the invention, or a pharmaceutically acceptable salt thereof. In certain aspects, the invention provides methods of treating fibrodysplasia ossificans progressive, comprising the step of administering to a subject in need thereof an amount of a compound of the invention, or a pharmaceutically acceptable salt thereof. In certain embodiments, the amount is an effective amount.

The present invention also provides a method of treating cancer, comprising the step of administering to a subject in need thereof a therapeutically effective amount of a compound of the invention, or a pharmaceutically acceptable salt thereof. In certain aspects, the invention provides a method of treating cancer, comprising the step of administering to a subject in need thereof an amount of a compound of the invention, or a pharmaceutically acceptable salt thereof. In certain embodiments, the amount is an effective amount.

In certain embodiments, the cancer comprises tumors of the central nervous system, breast cancer, prostate cancer, skin cancer (including basal cell carcinoma cell carcinoma, squamous cell carcinoma and melanoma), cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcoma, soft tissue sarcomas, osteosarcomas, peripheral primitive neuroectodermal tumors, retinoblastomas, rhabdomyosarcomas, and Wilms tumors.

In certain embodiments, the cancer is a glioma, such as diffuse intrinsic pontine glioma.

The present invention also provides a method of treating anemia associated with high hepcidin, Iron Refractory Iron Deficiency Anemia (IRIDA), anemia of chronic diseases, cancer-related anemia, chemotherapy-associated anemia, anemia of inflammation, or hepcidin-producing adenoma, comprising administering to a subject in need thereof a therapeutically effective amount of a compound of the invention, or a pharmaceutically acceptable salt thereof.

The present invention also provides a method of treating anemia associated with high hepcidin, Iron Refractory Iron Deficiency Anemia (IRIDA), anemia of chronic diseases, cancer-related anemia, chemotherapy-associated anemia, anemia of inflammation, or hepcidin-producing adenoma, comprising administering to a subject in need thereof an amount of a compound of the invention, or a pharmaceutically acceptable salt thereof. In certain embodiments, the amount is an effective amount.

In certain embodiments, the present disclosure provides methods of treating IRIDA.

The present invention also provides a method of treating spondyloarthritis (SpA), comprising administering to a subject in need thereof a therapeutically effective amount of a compound of the invention, or a pharmaceutically acceptable salt thereof.

The present invention provides a method of treating spondyloarthritis (SpA), comprising administering to a subject in need thereof an amount of a compound of the invention, or a pharmaceutically acceptable salt thereof. In certain embodiments, the amount is an effective amount.

The compounds of the invention are useful in treating any disease or condition whose treatment would benefit from ALK2 kinase inhibition, meaning that in such disease or condition it would be desirable to reduce ALK2 kinase activity. For example, it may be desirable to reduce ALK2 kinase activity in the setting of inappropriate activation or hyperactivation of ALK2 kinase.

In any of the foregoing, an additional pharmaceutically active agent other than a compound of the invention may also be administered to the subject.

### Formulations, Routes of Administration, and Dosing

The compounds of the invention, and pharmaceutically acceptable salts thereof, either alone or as a component of a pharmaceutical composition, can be administered to a mammalian host, such as a human patient, in a variety of forms adapted to the chosen route of administration, e.g., orally or parenterally, by intravenous, intraperitoneal, intramuscular, topical, or subcutaneous routes. Additional routes of administration are also contemplated by the invention.

Thus, the present compounds or pharmaceutically acceptable salts thereof may be systemically administered, e.g., orally, in combination with a pharmaceutically acceptable to vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound, or a pharmaceutically acceptable salt thereof, may be combined with one or more pharmaceutically acceptable carriers and used in the form of ingestible tablets, buccal tablets, troches, capsules, elixirs, suspensions, syrups, wafers, and the like. In some embodiments, such compositions and preparations contain at least 0.1% by weight of active compound, or a pharmaceutically acceptable salt thereof. The percentage of the active compound, or a pharmaceutically acceptable salt thereof, in such compositions and preparations may, of course, be varied and may conveniently be between about 2% to about 60% of the weight of a given unit dosage form. In some embodiments, the amount of active compound, or a pharmaceutically acceptable salt thereof, in such compositions is a therapeutically effective amount.

The tablets, troches, pills, capsules, and the like may also contain the following diluents and carriers: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, or a pharmaceutically acceptable salt thereof, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound, or a pharmaceutically acceptable salt thereof, may be incorporated into sustained-release preparations and devices.

The active compound, or a pharmaceutically acceptable salt thereof, may also be administered intravenously or intraperitoneally by infusion or injection. Solutions of the active compound, or a pharmaceutically acceptable salt thereof, can be prepared in water or physiologically acceptable aqueous solution, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active compound, or a pharmaceutically acceptable salt thereof, which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound, or a pharmaceutically acceptable salt thereof, in the required amount in the appropriate solvent with various of the other ingredients enumerated above, as required, followed by filter sterilization. In the case of sterile powders for the preparation of sterile injectable solutions, methods of preparation can include vacuum drying and the freeze drying techniques, which yield a powder of the active compound, or a pharmaceutically acceptable salt thereof, plus any additional desired ingredient present in the previously sterile-filtered solutions.

For topical administration, the active compound, or a pharmaceutically acceptable salt thereof, may be applied in pure form, i.e., when they are liquids. However, it will generally be desirable to administer them to the skin as compositions or formulations, in combination with a pharmaceutically acceptable carrier suitable for dermatologic use, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the active compound, or pharmaceutically acceptable salt thereof, can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention, or pharmaceutically acceptable salts thereof, to the skin are known in the art; for example, see Jacquet et al. (U.S. Pat. No. 4,608,392; incorporated herein by reference), Geria (U.S. Pat. No. 4,992,478; incorporated herein by reference), Smith et al. (U.S. Pat. No. 4,559,157; incorporated herein by reference), and Wortzman (U.S. Pat. No. 4,820,508; incorporated herein by reference).

Useful dosages of the active compound, or a pharmaceutically acceptable salt thereof, can be determined, at least initially, by comparing their in vitro activity and in vivo activity in animal models. Methods for the extrapolation of effective dosages in mice, and other animals, to humans are known in the art; for example, see U.S. Pat. No. 4,938,949 (incorporated herein by reference).

The amount of the active compound, or a pharmaceutically acceptable salt thereof, required for use in treatment will vary not only with the particular compound or salt selected but also with the route of administration, the nature of the condition being treated, and the age and condition of the patient and will be ultimately at the discretion of the attendant physician or clinician.

In general, however, a suitable dose will be in the range of from about 0.5 to about 100 mg/kg body weight of the recipient per day, e.g., from about 3 to about 90 mg/kg of body weight per day, from about 6 to about 75 mg per kilogram of body weight per day, from about of 10 to about 60 mg/kg of body weight per day, or from about 15 to about 50 mg/kg of body weight per day.

An active compound, or a pharmaceutically acceptable salt thereof, can be conveniently formulated in unit dosage form; for example, containing 5 to 1000 mg, 10 to 750 mg, or 50 to 500 mg of active compound, or a pharmaceutically acceptable salt thereof, per unit dosage form. In one embodiment, the invention provides a composition comprising an active compound, or pharmaceutically acceptable salt thereof, formulated in such a unit dosage form. The desired dose may conveniently be presented in a single dose or as divided doses to be administered at appropriate intervals, for example, as two, three, four or more sub-doses per day. The sub-dose itself may be further divided, e.g., into a number of discrete loosely spaced administrations.

An active compound, or a pharmaceutically acceptable salt thereof, can also be administered in combination with other therapeutic agents, for example, other agents that are useful for treating or preventing a disease or condition whose treatment would benefit from ALK2 kinase inhibition.

Other delivery systems can include time-release, delayed release, or sustained release delivery systems such as are well-known in the art. Such systems can avoid repeated administrations of the active compound, or a pharmaceutically acceptable salt thereof, increasing convenience to the subject and the physician. Many types of release delivery systems are available and known to those of ordinary skill in the art. Use of a long-term sustained release implant may be desirable. Long-term release, as used herein, means that the delivery system or is implant constructed and arranged to deliver therapeutic levels of the active compound, or a pharmaceutically acceptable salt thereof, for at least 30 days, and preferably 60 days.

In certain embodiments, an active compound, or pharmaceutically acceptable salt thereof, is formulated for intraocular administration, for example direct injection or insertion within or in association with an intraocular medical device.

An active compound, or a pharmaceutically acceptable salt thereof, may be formulated for depositing into a medical device, which may include any of a variety of conventional grafts, stents, including stent grafts, catheters, balloons, baskets, or other device that can be deployed or permanently implanted within a body lumen. As a particular example, it would be desirable to have devices and methods which can deliver compounds of the invention, or pharmaceutically acceptable salts thereof, to the region of a body which has been treated by interventional technique.

In exemplary embodiments, an active compound, or a pharmaceutically acceptable salt thereof, may be deposited within a medical device, such as a stent, and delivered to the treatment site for treatment of a portion of the body.

Stents have been used as delivery vehicles for therapeutic agents (i.e., drugs). Intravascular stents are generally permanently implanted in coronary or peripheral vessels.

Stent designs include those of U.S. Pat. No. 4,733,655 (Palmaz), U.S. Pat. No. 4,800,882 (Gianturco), or U.S. Pat. No. 4,886,062 (Wiktor). Such designs include both metal and polymeric stents, as well as self-expanding and balloon-expandable stents. Stents may also be used to deliver a drug at the site of contact with the vasculature, as disclosed in U.S. Pat. No. 5,102,417 (Palmaz), U.S. Pat. No. 5,419,760 (Narciso, Jr.), U.S. Pat. No. 5,429,634 (Narciso, Jr.), and in International Patent Application Nos. WO 91/12779 (Medtronic, Inc.) and WO 90/13332 (Cedars-Sanai Medical Center), for example.

The term “deposited” means that the active compound, or a pharmaceutically acceptable salt thereof, is coated, adsorbed, placed, or otherwise incorporated into the device by methods known in the art. For example, the active compound, or a pharmaceutically acceptable salt thereof, may be embedded and released from within (“matrix type”) or surrounded by and released through (“reservoir type”) polymer materials that coat or span the medical device. In the latter example, the active compound, or a pharmaceutically acceptable salt thereof, may be entrapped within the polymer materials or coupled to the polymer materials using one or more the techniques for generating such materials known in the art. In other formulations, the active compound, or a pharmaceutically acceptable salt thereof, may be linked to the surface of the medical device without the need for a coating, for example by means of detachable bonds, and release with time or can be removed by active mechanical or chemical processes. In other formulations, the active compound, or a pharmaceutically acceptable salt thereof, may be in a permanently immobilized form that presents the active compound at the implantation site.

In certain embodiments, the active compound, or a pharmaceutically acceptable salt thereof, may be incorporated with polymer compositions during the formation of biocompatible coatings for medical devices, such as stents. The coatings produced from these components are typically homogeneous and are useful for coating a number of devices designed for implantation.

The polymer may be either a biostable or a bioabsorbable polymer depending on the desired rate of release or the desired degree of polymer stability, but frequently a bioabsorbable polymer is suitable for this embodiment because, unlike a biostable polymer, it will typically not be present long after implantation to cause any adverse, chronic local response. Bioabsorbable polymers that could be used include, but are not limited to, poly(L-lactic acid), polycaprolactone, polyglycolide (PGA), poly(lactide-co-glycolide) (PLLA/PGA), poly(hydroxybutyrate), poly(hydroxybutyrate-co-valerate), polydioxanone, polyorthoester, polyanhydride, poly(glycolic acid), poly(D-lactic acid), poly(L-lactic acid), poly(D, L-lactic acid), poly(D, L-lactide) (PLA), poly (L-lactide) (PLLA), poly(glycolic acid-co-trimethylene carbonate) (PGA/PTMC), polyethylene oxide (PEO), polydioxanone (PDS), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid, polyepsilon caprolactone, polyhydroxy butyric acid, polyorthoesters, polyacetals, polydihydropyrans, polycyanoacrylates, cross linked or amphipathic block copolymers of hydrogels, and other suitable bioabsorbable polymers known in the art. Also, biostable polymers with a relatively low chronic tissue response such as polyurethanes, silicones, and polyesters could be used, and other polymers could also be used if they can be dissolved and cured or polymerized on the medical device such as polyolefins, polyisobutylene and ethylene-alphaolefin copolymers; acrylic polymers and copolymers, vinyl halide polymers and copolymers, such as polyvinyl chloride; polyvinylpyrrolidone; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; pyran copolymer; polyhydroxy-propyl-methacrylamide-phenol; polyhydroxyethyl-aspartamide-phenol; polyethyleneoxide-polylysine substituted with palmitoyl residues; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers, and carboxymethyl cellulose.

Polymers and semipermeable polymer matrices may be formed into shaped articles, such as valves, stents, tubing, prostheses and the like.

In certain embodiments of the invention, the compound of the invention, or pharmaceutically acceptable salt thereof, is coupled to a polymer or semipermeable polymer matrix that is formed as a stent or stent-graft device.

Typically, polymers are applied to the surface of an implantable device by spin coating, dipping, or spraying. Additional methods known in the art can also be utilized for this purpose. Methods of spraying include traditional methods as well as microdeposition techniques with an inkjet type of dispenser. Additionally, a polymer can be deposited on an implantable device using photo-patterning to place the polymer on only specific portions of the device. This coating of the device provides a uniform layer around the device which allows for improved diffusion of various analytes through the device coating.

In certain embodiments of the invention, the compound of the invention, or pharmaceutically acceptable salt thereof, is formulated for release from the polymer coating into the environment in which the medical device is placed. Preferably, the active compound, or a pharmaceutically acceptable salt thereof, is released in a controlled manner over an extended time frame (e.g., months) using at least one of several well-known techniques involving polymer carriers or layers to control elution. Some of these techniques are described in U.S. Patent Application 2004/0243225A1, the entire disclosure of which is incorporated herein in its entirety.

Moreover, as described for example in U.S. Pat. No. 6,770,729, which is incorporated herein in its entirety, the reagents and reaction conditions of the polymer compositions can be manipulated so that the release of the active compound, or a pharmaceutically acceptable salt thereof, from the polymer coating can be controlled. For example, the diffusion coefficient of the one or more polymer coatings can be modulated to control the release of the active compound, or a pharmaceutically acceptable salt thereof, from the polymer coating. In a variation on this theme, the diffusion coefficient of the one or more polymer coatings can be controlled to modulate the ability of an analyte that is present in the environment in which the medical device is placed (e.g., an analyte that facilitates the breakdown or hydrolysis of some portion of the polymer) to access one or more components within the polymer composition (and for example, thereby modulate the release of the active compound, or a pharmaceutically acceptable salt thereof, from the polymer coating). Yet another embodiment of the invention includes a device having a plurality of polymer coatings, each having a plurality of diffusion coefficients. In such embodiments of the invention, the release of the active compound, or a pharmaceutically acceptable salt thereof, from the polymer coating can be modulated by the plurality of polymer coatings.

In yet another embodiment of the invention, the release of the active compound, or a pharmaceutically acceptable salt thereof, from the polymer coating is controlled by modulating one or more of the properties of the polymer composition, such as the presence of one or more endogenous or exogenous compounds, or alternatively, the pH of the polymer composition. For example, certain polymer compositions can be designed to release an active compound, or a pharmaceutically acceptable salt thereof, in response to a decrease in the pH of the polymer composition.

### Kits

Kits comprising a compound of the invention are also provided. In one embodiment, a kit is provided comprising a compound of the invention, or a pharmaceutically acceptable salt thereof, and at least one of packaging material, and instructions for administering the compound of the invention or the pharmaceutically acceptable salt thereof and the other therapeutic agent or agents to a mammal to treat or prevent a disease or condition that would benefit from ALK2 inhibition. In one embodiment, the mammal is a human. In a specific embodiment, the mammal is a human.

In another embodiment, a kit is provided comprising a compound of the invention, or a pharmaceutically acceptable salt thereof, and at least one of at least one other therapeutic agent, packaging material, and instructions for administering the compound of the invention or the pharmaceutically acceptable salt thereof and the other therapeutic agent or agents to a mammal to treat or prevent a disease or condition that would benefit from ALK2 inhibition. In a specific embodiment, the mammal is a human.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof.

### EXAMPLES

Having now described the present invention in detail, the same will be more clearly understood by reference to the following examples, which are included herewith for purposes of illustration only and are not intended to be limiting of the invention.

For purposes of the present invention, the numerical descriptors “pyrrolo[2,1-f][1,2,4]triazine” and “pyrrolo[1,2-f][1,2,4]triazine” and the like in the context of a chemical name provided for a compound disclosed herein are understood to be synonymous and, therefore, may be and sometimes are used interchangeably. As a non-limiting example, the chemical names “2,4-dichloropyrrolo[2,1-f][1,2,4]triazine” and “2,4-dichloropyrrolo[1,2-f][1,2,4]triazine” are both understood to refer to a compound having the following structure:

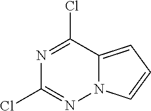 [SMILES: Clc1nc(Cl)c2cccn2n1]

As another non-limiting example, the chemical names “2-chloro-N-(1-methyl-1H-imidazol-4-yl)pyrrolo[2,1-f][1,2,4]triazin-4-amine” and “2-chloro-N-(1-methyl-1H-imidazol-4-yl)pyrrolo[1,2-f][1,2,4]triazin-4-amine” are both understood to refer to a compound having the following structure:

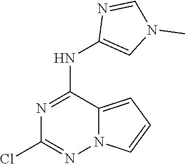 [SMILES: Cn1cnc(Nc2nc(Cl)nn3cccc23)c1]

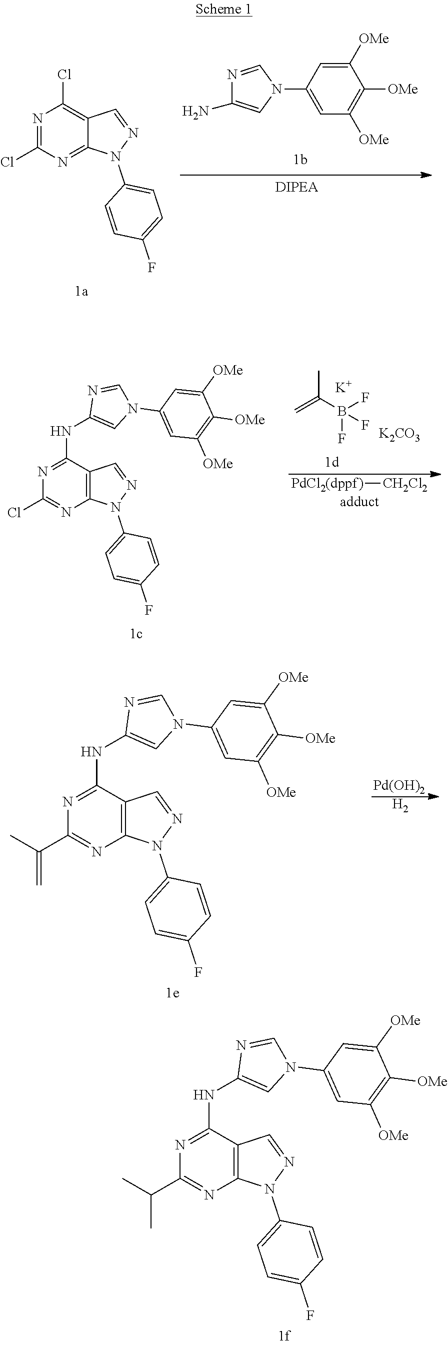

### Preparation of 1-(4-fluorophenyl)-6-isopropyl-N-(1-(3,4,5-trimethoxyphenyl)-1H-imidazol-4-yl)-1H-pyrazolo[3,4-d]pyrimidin-4-amine (1f)

### Step-1: Preparation of 6-chloro-1-(4-fluorophenyl)-N-(1-(3,4,5-trimethoxyphenyl)-1H-imidazol-4-yl)-1H-pyrazolo[3,4-d]pyrimidin-4-amine (1c)

To a solution of 4,6-dichloro-1-(4-fluorophenyl)-1H-pyrazolo[3,4-d]pyrimidine (Ia) (3 g, 10.60 mmol; CAS #1251465-40-3) in 2-Propanol (20 mL) was added DIPEA (5.55 mL, 31.8 mmol), 1-(3,4,5-trimethoxyphenyl)-1H-imidazol-4-amine (1b) (2.77 g, 11.13 mmol; prepared according to the procedure reported in Kotian, P. L. et al. PCT Int. Appl. (2018), WO to 2018/232094 A1; 20181220; incorporated by reference) and heated at reflux for 2 h. The reaction mixture was cooled down slowly by adding icy water and the solid obtained was collected by filtration to afford 6-chloro-1-(4-fluorophenyl)-N-(1-(3,4,5-trimethoxyphenyl)-1H-imidazol-4-yl)-1H-pyrazolo[3,4-d]pyrimidin-4-amine (1c) (3.6 g, 69% yield) as a yellow solid; ¹H NMR (300 MHz, DMSO-d₆) δ 11.56 (s, 1H, D₂O exchangeable), 8.71 (s, 1H), 8.21 (s, 1H), 8.09 (dd, J=8.6, 5.0 Hz, 2H), 7.90 (d, J=1.6 Hz, 1H), 7.50-7.38 (m, 2H), 6.94 (s, 2H), 3.88 (s, 6H), 3.70 (s, 3H); MS (ES+): 496.10 (M+1); (ES−): 494.10 (M−1).

### Step-2: Preparation of 1-(4-fluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (1e)

To a degassed solution of 6-chloro-1-(4-fluorophenyl)-N-(1-(3,4,5-trimethoxyphenyl)-1H-imidazol-4-yl)-1H-pyrazolo[3,4-d]pyrimidin-4-amine (1c) (364 mg, 2.63 mmol) in DMF/H₂O (5 mL, ratio: 4:1) was added potassium isopropenyltrifluoroborate (1d) (273 mg, 1.842 mmol; CAS #395083-14-4), potassium carbonate (364 mg, 2.63 mmol), PdCl₂(dppf)-CH₂Cl₂ adduct (172 mg, 0.211 mmol) and the resulting mixture was heated at 150° C. for 1 h in a microwave. The reaction was diluted with EtOAc, washed with water, brine, dried, filtered and concentrated in vacuum. The residue obtained was purified using flash column chromatography [silica gel (24 g), eluting with DMA-80 in DCM from 0-80%] to give 1-(4-fluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (1e) (243 mg, 46% yield); ¹H NMR (300 MHz, DMSO-d₆) δ 11.12 (s, 1H), 8.70 (s, 1H), 8.36-8.28 (m, 2H), 8.26 (d, J=1.6 Hz, 1H), 8.12 (s, 1H), 7.49-7.38 (m, 2H), 6.96 (s, 2H), 6.59-6.48 (m, 1H), 5.66-5.57 (m, 1H), 3.88 (s, 6H), 3.70 (s, 3H), 2.33 (s, 3H).

### Step-3: Preparation of 1-(4-fluorophenyl)-6-isopropyl-N-(1-(3,4,5-trimethoxyphenyl)-1H-imidazol-4-yl)-1H-pyrazolo[3,4-d]pyrimidin-4-amine (1f)

To a degassed solution of 1-(4-fluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (1e) (240 mg, 0.48 mmol) in MeOH (50 mL) was added palladium hydroxide on carbon (13.44 mg, 0.096 mmol). The resulting mixture was stirred for 12 h at RT under a H₂ atmosphere with a H₂ balloon (48.2 mg, 23.93 mmol). The reaction was then back filled with Ar and filtered through a short pad of Celite. The solvent was removed, the obtained residue was purified by reverse phase column chromatography [C18 column, eluting with ACN in water (containing 0.1% HCl) from 0-100%] to give 1-(4-fluorophenyl)-6-isopropyl-N-(1-(3,4,5-trimethoxyphenyl)-1H-imidazol-4-yl)-1H-pyrazolo[3,4-d]pyrimidin-4-amine (1f) (45 mg, 19% yield) HCl salt as a white solid; ¹H NMR (300 MHz, DMSO-d₆) δ 11.60 (s, 1H, D₂O exchangeable), 8.65 (s, 2H), 8.30-8.21 (m, 2H), 8.17 (d, J=1.6 Hz, 1H), 7.42 (t, J=8.8 Hz, 2H), 7.02 (s, 2H), 3.88 (s, 6H), 3.71 (s, 3H), 3.23-3.09 (m, 1H), 1.39 (d, J=6.9 Hz, 6H); ¹⁹F NMR (282 MHz, DMSO-d₆) δ −115.98; MS (ES+): 504.2 (M+1); (ES−): 502.2 (M−1); Analysis calculated for C₂₆H₂₆FN₇O₃·HCl·H₂O: C, 55.96; H, 5.24; Cl, 6.35; N, 17.57; Found: C, 56.04; H, 5.44; Cl, 6.55; N, 17.52.

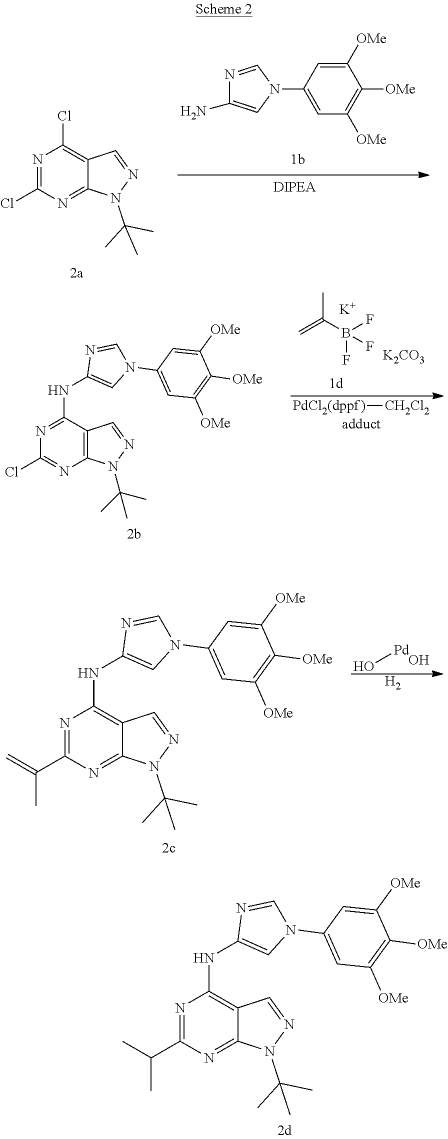

### Preparation of 1-(tert-butyl)-6-isopropyl-N-(1-(3,4,5-trimethoxyphenyl)-1H-imidazol-4-yl)-1H-pyrazolo[3,4-d]pyrimidin-4-amine (2d)

### Step-1: Preparation of 1-(tert-butyl)-6-chloro-N-(1-(3,4,5-trimethoxyphenyl)-1H-imidazol-4-yl)-1H-pyrazolo[3,4-d]pyrimidin-4-amine (2b)

Compound 2b was prepared according to the procedure reported in step-1 of scheme 1, from 1-(tert-butyl)-4,6-dichloro-1H-pyrazolo[3,4-d]pyrimidine (2a) (7.8 g, 31.8 mmol; CAS #864292-49-9) in 2-propanol (40 mL) using DIPEA (16.67 mL, 95 mmol), 1-(3,4,5-trimethoxyphenyl)-1H-imidazol-4-amine (1b) (7.93 g, 31.8 mmol) and refluxing for 3 h. This gave after work up 1-(tert-butyl)-6-chloro-N-(1-(3,4,5-trimethoxyphenyl)-1H-imidazol-4-yl)-1H-pyrazolo[3,4-d]pyrimidin-4-amine (2b) (8.9 g, 61%) as a yellow solid; ¹H NMR (300 MHz, DMSO-d₆) δ 11.31 (s, 1H, D₂O exchangeable), 8.44 (s, 1H), 8.19 (s, 1H), 7.88 (d, J=1.6 Hz, 1H), 6.93 (s, 2H), 3.88 (s, 6H), 3.70 (s, 3H), 1.70 (s, 9H); MS (ES+): 458.10 (M+1); (ES−): 456.10 (M+1).

### Step-2: Preparation of 1-(tert-butyl)-6-(prop-1-en-2-yl)-N-(1-(3,4,5-trimethoxyphenyl)-1H-imidazol-4-yl)-1H-pyrazolo[3,4-d]pyrimidin-4-amine (2c)

Compound 2c was prepared according to the procedure reported in step-2 of scheme 1, from 1-(tert-butyl)-6-chloro-N-(1-(3,4,5-trimethoxyphenyl)-1H-imidazol-4-yl)-1H-pyrazolo[3,4-d]pyrimidin-4-amine (2b) (1 g, 2.184 mmol) in dioxane/H₂O (27 mL, ratio: 8:1) using potassium isopropenyltrifluoroborate (1d) (485 mg, 3.28 mmol), potassium carbonate (604 mg, 4.37 mmol), PdCl₂(dppf)-CH₂Cl₂ adduct (268 mg, 0.328 mmol) and heating at 100° C. for 10 h. This gave after work up and purification using flash column chromatography [silica gel (24 g), eluting with DMA80 in DCM from 0-80%] 1-(tert-butyl)-6-(prop-1-en-2-yl)-N-(1-(3,4,5-trimethoxyphenyl)-1H-imidazol-4-yl)-1H-pyrazolo[3,4-d]pyrimidin-4-amine (2c) (620 mg, 61%) as a solid; ¹H NMR (300 MHz, DMSO-d₆) δ 10.84 (s, 1H), 8.40 (s, 1H), 8.24 (d, J=1.6 Hz, 1H), 8.10 (d, J=1.6 Hz, 1H), 6.94 (s, 2H), 6.54-6.38 (m, 1H), 5.56 (dd, J=2.8, 1.6 Hz, 1H), 3.88 (s, 6H), 3.70 (s, 3H), 2.29 (s, 3H), 1.75 (s, 9H).

### Step-3: Preparation of 1-(tert-butyl)-6-isopropyl-N-(1-(3,4,5-trimethoxyphenyl)-1H-imidazol-4-yl)-1H-pyrazolo[3,4-d]pyrimidin-4-amine (2d)

Compound 2d was prepared according to the procedure reported in step-3 of scheme 1, from 1-(tert-butyl)-6-(prop-1-en-2-yl)-N-(1-(3,4,5-trimethoxyphenyl)-1H-imidazol-4-yl)-1H-pyrazolo[3,4-d]pyrimidin-4-amine (2c) (278 mg, 0.60 mmol) in MeOH/DCM (110 mL, Ratio: 10:1) using palladium hydroxide on carbon, 20 wt. % loading (dry basis), matrix carbon, wet support (63.2 mg, 0.09 mmol) and stirring at RT for 12 h under a H₂ atmosphere. This gave after work up and purification using flash column chromatography [silica gel (24 g), eluting with DMA-80 in DCM from 0-80%] followed by purification using reverse phase column chromatography [C18 column, eluting with ACN in water (containing 0.1% HCl) from 0-100%] 1-(tert-butyl)-6-isopropyl-N-(1-(3,4,5-trimethoxyphenyl)-1H-imidazol-4-yl)-1H-pyrazolo[3,4-d]pyrimidin-4-amine (2d) (111 mg, 40% yield) HCl salt as a white solid; ¹H NMR (300 MHz, DMSO-d₆) δ 11.83 (s, 1H, D₂O exchangeable), 8.61 (s, 1H), 8.42 (s, 1H), 8.07 (d, J=1.7 Hz, 1H), 7.02 (s, 2H), 3.89 (s, 6H), 3.71 (s, 3H), 3.22-3.11 (m, 1H), 1.75 (s, 9H), 1.38 (d, J=6.9 Hz, 6H); MS (ES+), 466.2 (M+1); (ES−), 464.2 (M−1); Calculated for C₂₄H₃₁N₇O₃·(HCl)·1.5 (H₂O): C, 54.49; H, 6.67; Cl, 6.70; N, 18.53; Found: C, 54.64; H, 6.49; Cl, 6.58; N, 18.52.

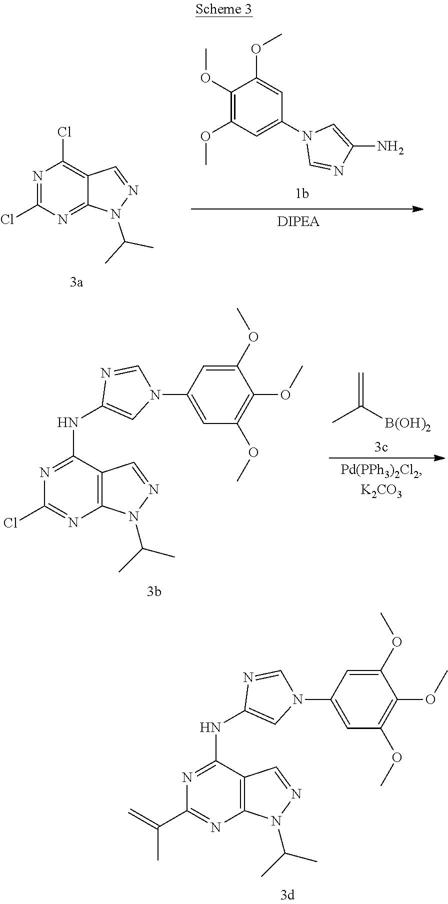

### Preparation of 1-isopropyl-6-(prop-1-en-2-yl)-N-(1-(3,4,5-trimethoxyphenyl)-1H-imidazol-4-yl)-1H-pyrazolo[3,4-d]pyrimidin-4-amine (3d)

### Step-1: Preparation of 6-chloro-1-isopropyl-N-(1-(3,4,5-trimethoxyphenyl)-1H-imidazol-4-yl)-1H-pyrazolo[3,4-d]pyrimidin-4-amine (3b)

Compound 3b was prepared according to the procedure reported in step-1 of scheme 1, from 4,6-dichloro-1-isopropyl-1H-pyrazolo[3,4-di]pyrimidine (3a) (758 mg, 3.28 mmol; CAS #21254-22-8) in 2-propanol (25 mL) using DIPEA (2 mL, 11.45 mmol) and 1-(3,4,5-trimethoxyphenyl)-1H-imidazol-4-amine (1b) (815 mg, 3.27 mmol) and heating at 90° C. for 2.5 h. This gave after work up 6-chloro-1-isopropyl-N-(1-(3,4,5-trimethoxyphenyl)-1H-imidazol-4-yl)-1H-pyrazolo[3,4-d]pyrimidin-4-amine (3b) (1.008 g, 70% yield) and was used as such for the next step; ¹H NMR (300 MHz, DMSO-d₆) δ 11.37 (s, 1H), 8.46 (s, 1H), 8.18 (s, 1H), 7.87 (d, J=1.6 Hz, 1H), 6.93 (s, 2H), 5.04-4.89 (m, 1H), 3.87 (s, 6H), 3.70 (s, 3H), 1.45 (d, J=6.7 Hz, 6H).

### Step-2: Preparation of 1-isopropyl-6-(prop-1-en-2-yl)-N-(1-(3,4,5-trimethoxyphenyl)-1H-imidazol-4-yl)-1H-pyrazolo[3,4-d]pyrimidin-4-amine (3d)

To a solution of 6-chloro-1-isopropyl-N-(1-(3,4,5-trimethoxyphenyl)-1H-imidazol-4-yl)-1H-pyrazolo[3,4-d]pyrimidin-4-amine (3b) (303 mg, 0.683 mmol) and prop-1-en-2-ylboronic acid (3c) (88 mg, 1.024 mmol) in dioxane (5 mL) was added a solution of potassium carbonate (283 mg, 2.048 mmol) in water (0.5 mL), bis(triphenylphosphine)palladium(II) chloride (96 mg, 0.137 mmol) and heated at 100° C. for 5 h under argon. The solvent was removed in vacuum and the residue obtained was purified by flash column chromatography [silica gel (12 g), eluting with DMA-80 in DCM from 0-70%] to provide 1-isopropyl-6-(prop-1-en-2-yl)-N-(1-(3,4,5-trimethoxyphenyl)-1H-imidazol-4-yl)-1H-pyrazolo[3,4-d]pyrimidin-4-amine (3d) (264 mg, 86% yield) as a yellow solid, 84 mgs of this solid was re-purified using reverse phase column chromatography [C18 column (50 g), eluting with ACN in water (containing 0.1% HCl) from 0-100%] to provide HCl salt of 1-isopropyl-6-(prop-1-en-2-yl)-N-(1-(3,4,5-trimethoxyphenyl)-1H-imidazol-4-yl)-1H-pyrazolo[3,4-d]pyrimidin-4-amine (3d) (49 mg) as a white solid; ¹H NMR (300 MHz, DMSO-d₆) δ 11.37 (s, 1H), 8.64 (s, 1H), 8.39 (s, 1H), 8.08 (s, 1H), 6.97 (s, 2H), 6.51-6.14 (m, 1H), 5.71-5.38 (m, 1H), 5.09-4.96 (m, 1H), 3.82 (s, 6H), 3.64 (s, 3H), 2.22 (s, 3H), 1.58-1.22 (m, 6H); MS (ES+): 450.2 (M+1); (ES−): 448.2 (M−1); Analysis calculated for: C₂₃H₂₇N₇O₃·0.85HCl·1.5H₂O: C, 54.43; H, 6.13; Cl, 5.94; N, 19.32; Found: C, 54.23; H, 6.06; Cl, 5.99; N, 19.24.

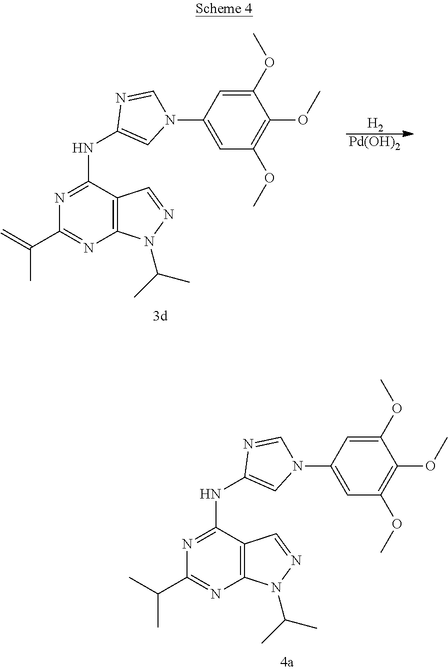

### Preparation of 1,6-diisopropyl-N-(1-(3,4,5-trimethoxyphenyl)-1H-imidazol-4-yl)-1H-pyrazolo[3,4-d]pyrimidin-4-amine (4a)

Compound 4a was prepared according to the procedure reported in step-3 of scheme 1, from 1-isopropyl-6-(prop-1-en-2-yl)-N-(1-(3,4,5-trimethoxyphenyl)-1H-imidazol-4-yl)-1H-pyrazolo[3,4-d]pyrimidin-4-amine (3d) (180 mg, 0.400 mmol) in MeOH (20 mL) using palladium hydroxide on carbon, 20 wt. % loading (dry basis), matrix carbon, wet support (50 mg, 0.071 mmol) and stirring overnight at RT under a H₂ atmosphere. This gave after work up and purification using reverse phase column chromatography [C18 column (50 g), eluting with ACN in water (containing 0.1% HCl) from 0-100%] 1,6-diisopropyl-N-(1-(3,4,5-trimethoxyphenyl)-1H-imidazol-4-yl)-1H-pyrazolo[3,4-d]pyrimidin-4-amine (4a) (119 mg, 66% yield) HCl salt as a white solid; ¹H NMR (300 MHz, DMSO-d₆) δ 12.12 (s, 1H), 8.60 (s, 1H), 8.52 (s, 1H), 8.07 (d, J=1.7 Hz, 1H), 7.01 (s, 2H), 5.21-5.02 (m, 1H), 3.88 (s, 6H), 3.70 (s, 3H), 3.30-3.08 (m, 1H), 1.47 (d, J=6.6 Hz, 6H), 1.39 (d, J=6.9 Hz, 6H); MS (ES+): 452.2 (M+1); (ES−): 450.2 (M−1); Analysis calculated for: C₂₁H₂₉N₇O₃·0.85HCl·1.75H₂O: C, 53.74; H, 6.54; Cl, 5.86; N, 19.07; Found: C, 53.79; H, 6.61; Cl, 6.00; N, 18.92.

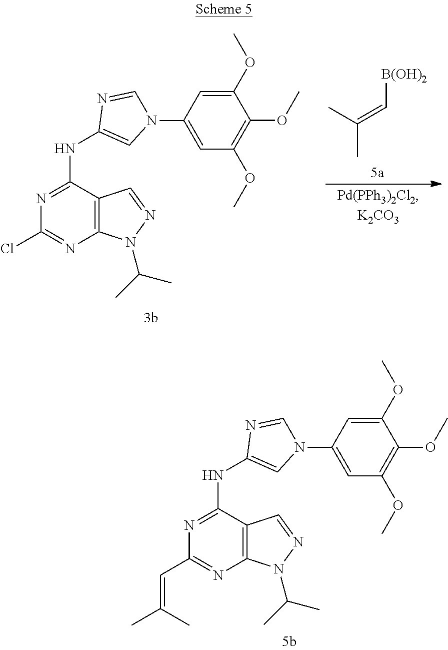

### Preparation of 1-isopropyl-6-(2-methylprop-1-en-1-yl)-N-(1-(3,4,5-trimethoxyphenyl)-1H-imidazol-4-yl)-1H-pyrazolo[3,4-d]pyrimidin-4-amine (5b)

Compound 5b was prepared according to the procedure reported in step-2 of scheme 3, from 6-chloro-1-isopropyl-N-(1-(3,4,5-trimethoxyphenyl)-1H-imidazol-4-yl)-1H-pyrazolo[3,4-d]pyrimidin-4-amine (3b) (306 mg, 0.689 mmol) in dioxane (5 mL) using (2-methylprop-1-en-1-yl)boronic acid (5a) (103 mg, 1.034 mmol), a solution of potassium carbonate (286 mg, 2.068 mmol) in water (0.5 mL), bis(triphenylphosphine)palladium(II) chloride (96 mg, 0.138 mmol) and heating at 100° C. for 5 h under argon. This gave after work up and purification using flash column chromatography [silica gel (24 g), eluting with DMA-80 in DCM from 0-90%]-isopropyl-6-(2-methylprop-1-en-1-yl)-N-(1-(3,4,5-trimethoxyphenyl)-1H-imidazol-4-yl)-1H-pyrazolo[3,4-d]pyrimidin-4-amine (5b) (320 mg, 100% yield) as a yellow solid, 84 mgs of this solid was further purified using the reverse phase column chromatography [C18 (50 g), eluting with ACN in water (containing 0.1% HCl) from 0-100%] to provide 1-isopropyl-6-(2-methylprop-1-en-1-yl)-N-(1-(3,4,5-trimethoxyphenyl)-1H-imidazol-4-yl)-1H-pyrazolo[3,4-d]pyrimidin-4-amine (5b) HCl salt (44 mgs) as a white solid; ¹H NMR (300 MHz, DMSO-d₆) δ 11.65 (s, 1H), 8.60 (s, 1H), 8.45 (s, 1H), 8.02 (d, J=1.6 Hz, 1H), 7.01 (s, 2H), 6.40 (s, 1H), 5.15-4.94 (m, 1H), 3.89 (s, 6H), 3.70 (s, 3H), 2.40-2.24 (m, 3H), 2.06-1.89 (m, 3H), 1.48 (d, J=6.7 Hz, 6H); MS (ES+): 464.3 (M+1); MS (ES−): 462.2 (M−1); Analysis calculated for C₂₄H₂₉N₇O₃·0.85HCl·1.75H₂O: C, 54.80; H, 6.39; Cl, 5.73; N, 18.64; Found: C, 54.86; H, 6.28; Cl, 5.49; N, 18.54.

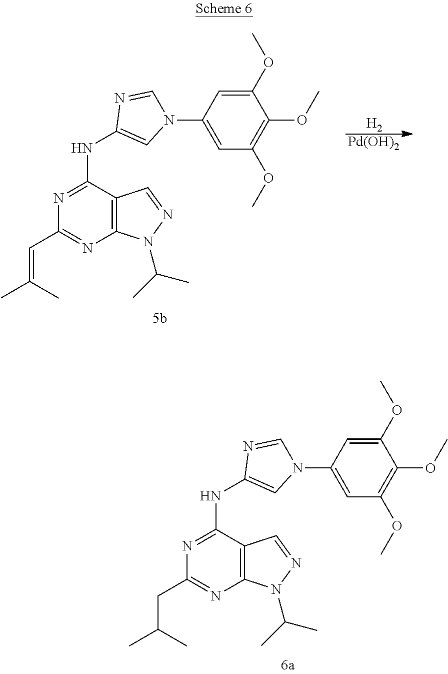

### Preparation of 6-isobutyl-1-isopropyl-N-(1-(3,4,5-trimethoxyphenyl)-1H-imidazol-4-yl)-1H-pyrazolo[3,4-d]pyrimidin-4-amine (6a)

Compound 6a was prepared according to the procedure reported in step-3 of scheme 1, from 1-isopropyl-6-(2-methylprop-1-en-1-yl)-N-(1-(3,4,5-trimethoxyphenyl)-1H-imidazol-4-yl)-1H-pyrazolo[3,4-d]pyrimidin-4-amine (5b) (240 mg, 0.518 mmol) in MeOH (20 mL) using palladium hydroxide on carbon, 20 wt. % loading (dry basis), matrix carbon, wet support (90 mg, 0.128 mmol) and stirring at RT for 3 days under a H₂ atmosphere. This gave after work up and purification using reverse phase column chromatography [C18 column (50 g), eluting with ACN in water (containing 0.1% HCl) from 0-100%] 6-isobutyl-1-isopropyl-N-(1-(3,4,5-trimethoxyphenyl)-1H-imidazol-4-yl)-1H-pyrazolo[3,4-d]pyrimidin-4-amine (6a) (92 mg, 38% yield) HCl salt as a white solid; ¹H NMR (300 MHz, DMSO-d %) δ 10.81 (s, 1H), 8.38 (s, 1H), 8.22 (d, J=1.6 Hz, 1H), 8.08 (s, 1H), 6.91 (s, 2H), 5.20-4.81 (m, 1H), 3.88 (s, 6H), 3.69 (s, 3H), 2.72 (d, J=7.1 Hz, 2H), 2.45-2.29 (m, 1H), 1.44 (d, J=6.7 Hz, 6H), 0.97 (d, J=6.6 Hz, 6H); MS (ES+): 466.3 (M+1); MS (ES−): 464.3 (M−1); Analysis calculated for C₂₄H₃₁N₇O₃: C, 61.92; H, 6.71; N, 21.06 Found: C, 61.82; H, 6.62; N, 20.91.

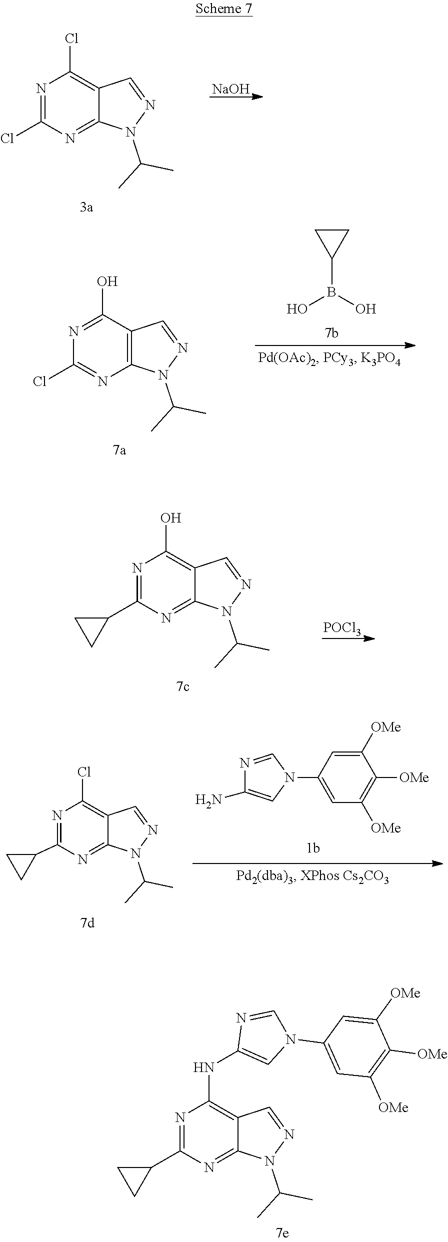

### Preparation of 6-cyclopropyl-1-isopropyl-N-(1-(3,4,5-trimethoxyphenyl)-1H-imidazol-4-yl)-1H-pyrazolo[3,4-d]pyrimidin-4-amine (7e)

### Step-1: Preparation of 6-chloro-1-isopropyl-1H-pyrazolo[3,4-d]pyrimidin-4-ol (7a)

A solution of 4,6-dichloro-1-isopropyl-1H-pyrazolo[3,4-d]pyrimidine (3a) (4.8 g, 20.77 mmol) in NaOH (3 N, 69.2 mL, 208 mmol) was heated to 60° C. for 1.5 h. The reaction mixture was cooled to RT and filtered. The filtrate was acidified with HCl (2 N) to PH-4 and the white solid obtained was collected by filtration to give 6-chloro-1-isopropyl-1H-pyrazolo[3,4-d]pyrimidin-4-ol (7a) (3.15 g, 71% yield) as a white solid which was used as such in the next step; MS (ES+): 213.05 (M+1); (ES−): 211.05 (M−1).

### Step-2: Preparation of 6-cyclopropyl-1-isopropyl-1H-pyrazolo[3,4-d]pyrimidin-4-ol (7c)

Compound 7c was prepared according to the procedure reported in step-1 of scheme 1, from 6-chloro-1-isopropyl-1H-pyrazolo[3,4-d]pyrimidin-4-ol (7a) (243 mg, 1.143 mmol) in toluene (6 mL) and water (0.6 mL) using cyclopropylboronic acid (7b)(196 mg, 2.286 mmol), palladium(II) acetate (25.7 mg, 0.114 mmol), tricyclohexylphosphine (64.1 mg, 0.229 mmol), K3P04 (606 mg, 2.86 mmol) and heating at 100° C. for 7 h under argon. This gave after workup and purification using flash column chromatography [silica gel (24 g), eluting with MeOH in EtOAc 1:9 in hexanes from 10-100%] 6-cyclopropyl-1-isopropyl-1H-pyrazolo[3,4-d]pyrimidin-4-ol (7c) (249 mg, 100% yield) as a yellow solid; MS (ES+): 219.10 (M+1).

### Step-3: Preparation of 4-chloro-6-cyclopropyl-1-isopropyl-1H-pyrazolo[3,4-d]pyrimidine (7d)

A mixture of 6-cyclopropyl-1-isopropyl-1H-pyrazolo[3,4-d]pyrimidin-4-ol (7c) (249 mg, 1.141 mmol) in POCl₃ (5 mL, 53.6 mmol) was heated at 100° C. for 1 h, cooled to room temperature and concentrated in vacuum to dryness. The residue obtained was purified by flash column chromatography [silica gel (12 g), eluting with EtOAc in hexane from 0-40%] to afford 4-chloro-6-cyclopropyl-1-isopropyl-1H-pyrazolo[3,4-di]pyrimidine (7d) (196 mg, 73% yield); ¹H NMR (300 MHz, DMSO-d₆) δ 8.31 (s, 1H), 5.14-4.97 (m, 1H), 2.33-2.20 (m, 1H), 1.50 (d, J=3.1 Hz, 3H), 1.48 (d, J=3.1 Hz, 3H), 1.17-1.05 (m, 4H); MS (ES+): 237.10 (M+1).

### Step-4: Preparation of 6-cyclopropyl-1-isopropyl-N-(1-(3,4,5-trimethoxyphenyl)-1H-imidazol-4-yl)-1H-pyrazolo[3,4-d]pyrimidin-4-amine (7e)

To a degassed solution of XPhos (155 mg, 0.324 mmol) in toluene/t-Butanol (10 mL, ratio: 4:1) was added cesium carbonate (661 mg, 2.028 mmol), Pd₂(dba)₃ (149 mg, 0.162 mmol), 4-chloro-6-cyclopropyl-1-isopropyl-1H-pyrazolo[3,4-d]pyrimidine (7d) (192 mg, 0.811 mmol), 1-(3,4,5-trimethoxyphenyl)-1H-imidazol-4-amine (1b) (404 mg, 1.622 mmol) and heated at 110° C. for 4 h under argon. The solvent was evaporated and the residue obtained was purified using flash column chromatography [silica gel (12 g), eluting with MeOH in DCM from 0-15%] followed by purification using reverse phase column chromatography [C18 column (50 g), eluting with ACN in water (containing 0.1% HCl) from 0-100%] to give 6-cyclopropyl-1-isopropyl-N-(1-(3,4,5-trimethoxyphenyl)-1H-imidazol-4-yl)-1H-pyrazolo[3,4-d]pyrimidin-4-amine (7e) (106 mg, 29% yield) HCl salt as a white solid; ¹H NMR (300 MHz, DMSO-d₆) δ 11.69 (s, 1H), 8.65 (s, 1H), 8.42 (s, 1H), 7.99 (d, J=1.6 Hz, 1H), 7.03 (s, 2H), 5.17-5.08 (m, 1H), 3.90 (s, 6H), 3.71 (s, 3H), 2.42-2.17 (m, 1H), 1.46 (d, J=6.6 Hz, 6H), 1.34-1.19 (m, 2H), 1.19-1.00 (m, 2H); MS (ES+): 450.2 (M+1); (ES−): 448.2 (M−1); Analysis calculated for: C₂₃H₂₇N₇O₃·0.95HCl·1.9H₂O: C, 53.29; H, 6.17; Cl, 6.50; N, 18.91; Found: C, 53.46; H, 6.05; Cl, 6.54; N, 18.64.

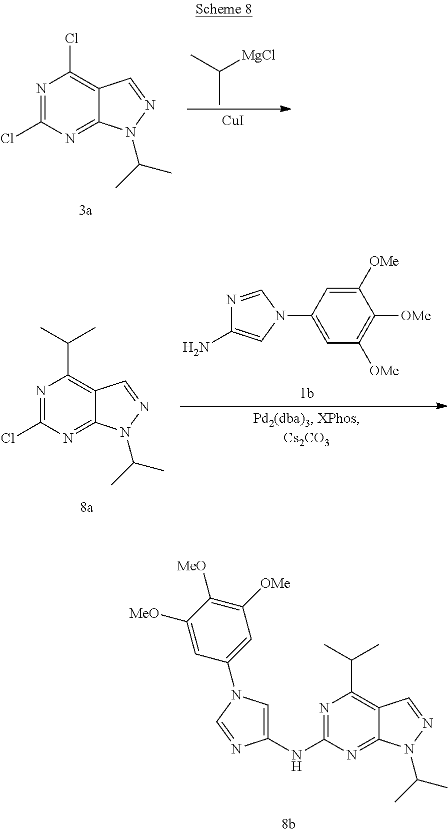

### Preparation of 1,4-diisopropyl-N-(1-(3,4,5-trimethoxyphenyl)-1H-imidazol-4-yl)-1H-pyrazolo[3,4-d]pyrimidin-6-amine (8b)

### Step-1: Preparation of 6-chloro-1,4-diisopropyl-1H-pyrazolo[3,4-d]pyrimidine (8a)

Nitrogen was bubbled to a solution of 4,6-dichloro-1-isopropyl-1H-pyrazolo[3,4-d]pyrimidine (3a) (500 mg, 2.164 mmol) and copper (I) iodide (20.60 mg, 0.108 mmol) in THF (5 mL) at −20° C. for 10 minutes, added dropwise isopropylmagnesiumchloride (2.380 mL, 4.76 mmol) and stirred at RT until the reaction was complete. The reaction was carefully diluted with saturated ammonium chloride and the aqueous layer was extracted with ethyl acetate (3×20 mL). The combined organics were dried, filtered, and concentrated in vacuum and the residue obtained was purified using flash column chromatography [silica gel (12 g), eluting with EtOAc in hexane from 0-100%] to give 6-chloro-1,4-diisopropyl-1H-pyrazolo[3,4-d]pyrimidine (8a) (232 mg, 45% yield); ¹H NMR (300 MHz, DMSO-d₆) δ 8.58 (s, 1H), 5.14-4.93 (m, 1H), 3.58-3.36 (m, J=7.0 Hz, 1H), 1.52-1.42 (m, 6H), 1.46-1.28 (m, 6H).

### Step-2: Preparation of 1,4-diisopropyl-N-(1-(3,4,5-trimethoxyphenyl)-1H-imidazol-4-yl)-1H-pyrazolo[3,4-d]pyrimidin-6-amine (8b)

Compound 8b was prepared according to the procedure reported in step-4 of scheme 7, from 6-chloro-1,4-diisopropyl-1H-pyrazolo[3,4-d]pyrimidine (8a) (563 mg, 0.9718 mmol) in toluene/t-Butanol (25 mL, ratio: 4:1) using XPhos (185 mg, 0.389 mmol), cesium carbonate (1108 mg, 3.40 mmol), Pd₂(dba)₃ (178 mg, 0.194 mmol), 1-(3,4,5-trimethoxyphenyl)-1H-imidazol-4-amine (1b) (291 mg, 1.166 mmol) and heating at 110° C. for 12 hrs. This gave after workup and purification using flash column chromatography [silica gel (24 g), eluting with DMA-80 in DCM from 0-50%] followed by purification using reverse phase column chromatography [C18 steel column, eluting with ACN in water (containing 0.1% HCl) from 0-100%] 1,4-diisopropyl-N-(1-(3,4,5-trimethoxyphenyl)-1H-imidazol-4-yl)-1H-pyrazolo[3,4-d]pyrimidin-6-amine (8b) (12 mg, 2% yield) HCl salt as a reddish solid. 1H NMR (300 MHz, DMSO-d₆) δ 10.46 (s, 1H), 9.20-9.10 (m, 1H), 8.30 (s, 1H), 8.12-7.97 (m, 1H), 7.12 (d, J=2.4 Hz, 2H), 5.14-4.98 (m, 1H), 3.90 (s, 6H), 3.72 (s, 3H), 3.49-3.33 (m, 1H), 1.52-1.38 (m, 6H), 1.38 (d, J=6.9 Hz, 6H); MS (ES+): 452.2 (M+1).

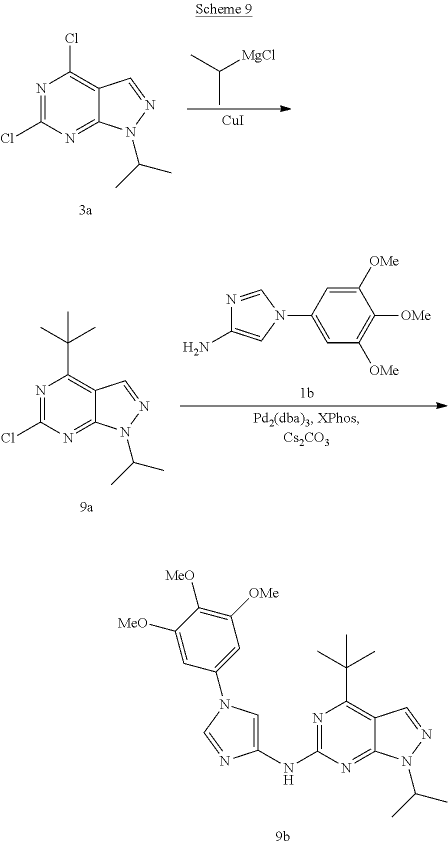

### Preparation of 4-(tert-butyl)-1-isopropyl-N-(1-(3,4,5-trimethoxyphenyl)-1H-imidazol-4-yl)-1H-pyrazolo[3,4-d]pyrimidin-6-amine (9b)

### Step-1: Preparation of 4-(tert-butyl)-6-chloro-1-isopropyl-1H-pyrazolo[3,4-d]pyrimidine (9a)

Compound 9a was prepared according to the procedure reported in step-1 of scheme 8, from 4,6-dichloro-1-isopropyl-1H-pyrazolo[3,4-d]pyrimidine (3a) (1 g, 4.33 mmol) in THF (10 mL) using tert-butyl magnesium chloride (4.76 mL, 9.52 mmol) and copper (I) iodide (41 mg, 0.216 mmol). This gave after workup and purification using flash column chromatography [silica gel (12 g), eluting with EtOAc in hexane from 0-100%] 4-(tert-butyl)-6-chloro-1-isopropyl-1H-pyrazolo[3,4-d]pyrimidine (9a) (767 mg, 70% yield); ¹H NMR (300 MHz, DMSO-d₆) δ 8.64 (d, J=0.6 Hz, 1H), 5.16-4.97 (m, 1H), 1.47 (s, 15H); MS (ES+): 253.10 (M+1).

### Step-2: Preparation of 4-(tert-butyl)-1-isopropyl-N-(1-(3,4,5-trimethoxyphenyl)-1H-imidazol-4-yl)-1H-pyrazolo[3,4-d]pyrimidin-6-amine (9b)

Compound 9b was prepared according to the procedure reported in step-3 of scheme 100, from 4-(tert-butyl)-6-chloro-1-isopropyl-1H-pyrazolo[3,4-d]pyrimidine (9a) (1757 mg, 3.034 mmol) in toluene (20 mL) and t-butanol (5 mL) using 1-(3,4,5-trimethoxyphenyl)-1H-imidazol-4-amine (1b) (908 mg, 3.64 mmol), XPhos (579 mg, 1.214 mmol), cesium carbonate (3460 mg, 10.62 mmol), Pd₂(dba)₃ (556 mg, 0.607 mmol) and heating at 110° C. for 12 h. This gave after work up and purification using flash column chromatography [silica gel (24 g), eluting with DMA-80 in DCM from 0-50%] followed by purification using reverse phase column chromatography [C18 column, eluting with ACN in water (containing 0.1% HCl) from 0-100%] 4-(tert-butyl)-1-isopropyl-N-(1-(3,4,5-trimethoxyphenyl)-1H-imidazol-4-yl)-1H-pyrazolo[3,4-d]pyrimidin-6-amine (9b) (48 mg, 2% yield) HCl salt as a light yellow; ¹H NMR (300 MHz, DMSO-d₆) δ 10.07 (s, 1H), 8.59 (s, 1H), 8.29 (s, 1H), 8.01 (d, J=1.8 Hz, 1H), 7.00 (s, 2H), 5.12-4.98 (m, 1H), 3.89 (s, 6H), 3.71 (s, 3H), 1.53-1.45 (m, 15H); MS (ES+): 466.2 (M+1).

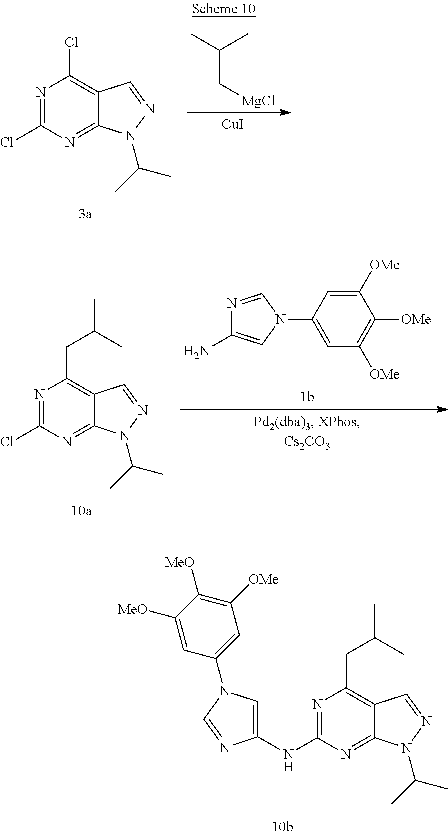

### Preparation of 4-isobutyl-1-isopropyl-N-(1-(3,4,5-trimethoxyphenyl)-1H-imidazol-4-yl)-1H-pyrazolo[3,4-d]pyrimidin-6-amine (10b)

### Step-1: Preparation of 6-chloro-4-isobutyl-1-isopropyl-1H-pyrazolo[3,4-d]pyrimidine (10a)

Compound 10a was prepared according to the procedure reported in step-1 of scheme 8, from 4,6-dichloro-1-isopropyl-1H-pyrazolo[3,4-d]pyrimidine (3a) (1 g, 4.33 mmol) in THF (10 mL) using isobutyl magnesium chloride (4.76 mL, 9.52 mmol) and copper (I) iodide (41 mg, 0.216 mmol) and stirring at RT until the reaction is complete. This gave after workup and purification using flash column chromatography [silica gel (12 g), eluting with EtOAc in hexane from 0-100%] 6-chloro-4-isobutyl-1-isopropyl-1H-pyrazolo[3,4-d]pyrimidine (10a) (318 mg, 29% yield); ¹H NMR (300 MHz, DMSO-d₆) δ 8.55 (s, 1H), 5.14-4.93 (m, 2H), 2.35-2.13 (m, 1H), 1.52-1.42 (m, 7H), 0.93 (d, J=6.6 Hz, 6H).

### Step-2: Preparation of 4-isobutyl-1-isopropyl-N-(1-(3,4,5-trimethoxyphenyl)-1H-imidazol-4-yl)-1H-pyrazolo[3,4-d]pyrimidin-6-amine (10b)

Compound 10b was prepared according to the procedure reported in step-4 of scheme 7, from 6-chloro-4-isobutyl-1-isopropyl-1H-pyrazolo[3,4-d]pyrimidine (10a) (729 mg, 1.259 mmol) in toluene (20 mL) and t-butanol (5 mL) using 1-(3,4,5-trimethoxyphenyl)-1H-imidazol-4-amine (1b) (377 mg, 1.511 mmol), cesium carbonate (1436 mg, 4.41 mmol), Pd₂(dba)₃ (231 mg, 0.252 mmol), XPhos (240 mg, 0.504 mmol) and heating at 110° C. for 12 h under argon. This gave after workup and purification using flash column chromatography [silica gel (24 g), eluting with DMA-80 in DCM from 0-50%] followed by purification using reverse phase column chromatography [C18 column (50 g), eluting with ACN in water (containing 0.1% HCl) from 0-100%] 4-isobutyl-1-isopropyl-N-(1-(3,4,5-trimethoxyphenyl)-1H-imidazol-4-yl)-1H-pyrazolo[3,4-d]pyrimidin-6-amine (10b) (45 mg, 4.5% yield) HCl salt as a light brown solid; ¹H NMR (300 MHz, DMSO-d₆) δ 10.18 (s, 1H, D₂O exchangeable), 8.56 (s, 1H), 8.19 (s, 1H), 8.00 (d, J=1.7 Hz, 1H), 7.00 (s, 2H), 5.11-4.96 (m, 1H), 3.89 (s, 6H), 3.71 (s, 3H), 2.83 (d, J=7.3 Hz, 2H), 2.35-2.20 (m, 1H), 1.50 (d, J=6.7 Hz, 6H), 0.96 (d, J=6.6 Hz, 6H); MS (ES+): 466.2 (M+1).

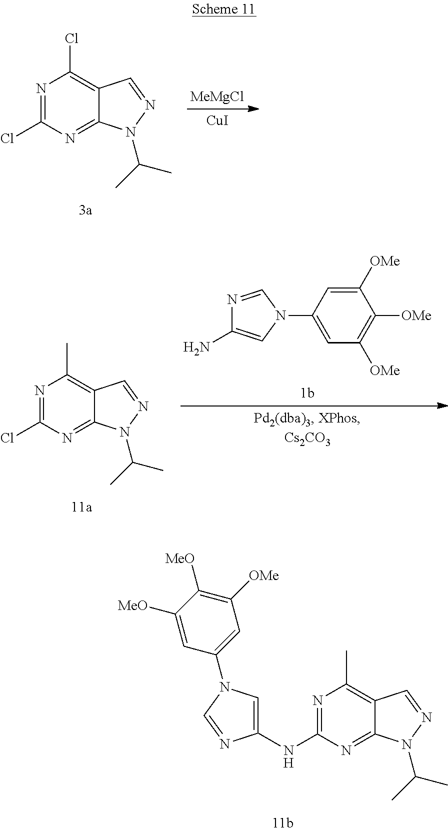 [SMILES: "MeMgCl" ; CC(C)n1ncc2c(Cl)nc(Cl)nc21 ; "CuI" ; Cc1nc(Cl)nc2c1cnn2C(C)C ; COc1cc(-n2cnc(N)c2)cc(OC)c1OC ; COc1cc(-n2cnc(Nc3nc(C)c4cnn(C(C)C)c4n3)c2)cc(OC)c1OC]

### Preparation of 1-isopropyl-4-methyl-N-(1-(3,4,5-trimethoxyphenyl)-1H-imidazol-4-yl)-1H-pyrazolo[3,4-d]pyrimidin-6-amine (11b)

### Step-1: Preparation of 6-chloro-1-isopropyl-4-methyl-1H-pyrazolo[3,4-d]pyrimidine (11a)

Compound 11a was prepared according to the procedure reported in step-1 of scheme 8, from 4,6-dichloro-1-isopropyl-1H-pyrazolo[3,4-d]pyrimidine (3a) (1 g, 4.33 mmol) in THF (20 mL) using methyl magnesium chloride (1.442 mL, 4.33 mmol) and copper (I) iodide (41 mg, 0.216 mmol). This gave after workup and purification using flash column chromatography [silica gel (12 g), eluting with EtOAc in hexane from 0-100%] 6-chloro-1-isopropyl-4-methyl-1H-pyrazolo[3,4-d]pyrimidine (11a) (86 mg, 9% yield); MS (ES+): 211.10 (M+1).

### Step-2: Preparation of 1-isopropyl-4-methyl-N-(1-(3,4,5-trimethoxyphenyl)-1H-imidazol-4-yl)-1H-pyrazolo[3,4-d]pyrimidin-6-amine (11b)

Compound 11b was prepared according to the procedure reported in step-4 of scheme 7, from 6-chloro-1-isopropyl-4-methyl-1H-pyrazolo[3,4-d]pyrimidine (11a) (0.236 g, 0.408 mmol) in toluene (20 mL) and t-butanol (5 mL), using 1-(3,4,5-trimethoxyphenyl)-1H-imidazol-4-amine (1b) (0.122 g, 0.49 mmol) cesium carbonate (0.465 g, 1.428 mmol), Pd₂(dba)₃ (0.075 g, 0.082 mmol), XPhos (0.078 g, 0.163 mmol) and stirring at 110° C. for 12 h under argon. This gave after workup and purification using flash column chromatography [silica gel (24 g), eluting with DMA-80 in DCM from 0-50%] followed by purification using reverse phase column chromatography [C18 column (50 g), eluting with ACN in water (containing 0.1% HCl) from 0-100%] 1-isopropyl-4-methyl-N-(1-(3,4,5-trimethoxyphenyl)-1H-imidazol-4-yl)-1H-pyrazolo[3,4-d]pyrimidin-6-amine (11b) (0.03 g, 9% yield) HCl salt as a white solid; ¹H NMR (300 MHz, DMSO-d₆) δ 10.57 (m, 1H, D₂O exchangeable), 9.27 (s, 1H), 8.27 (s, 1H), 8.15-8.06 (m, 1H), 7.15 (s, 2H), 5.07 (m, J=6.6 Hz, 1H), 3.90 (s, 6H), 3.72 (s, 3H), 2.69 (s, 3H), 1.49 (d, J=6.5, 4.5 Hz, 6H). MS (ES+): 424.2 (M+1); Analysis calculated for C₂₁H₂N₇O₃·1.5 HCl·2.25H₂O: C, 48.80; H, 6.04; Cl, 9.95; N, 18.97; Found: C, 48.76; H, 5.83; Cl, 9.80; N, 18.98.

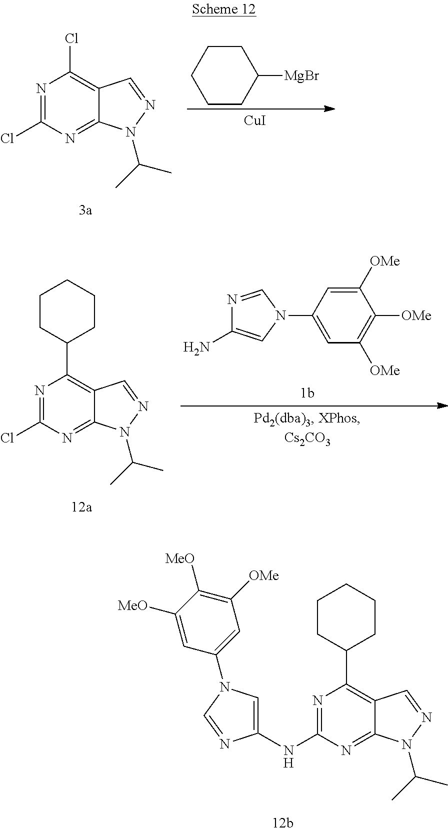

### Preparation of 4-cyclohexyl-1-isopropyl-N-(1-(3,4,5-trimethoxyphenyl)-1H-imidazol-4-yl)-1H-pyrazolo[3,4-d]pyrimidin-6-amine (12b)

### Step-1: Preparation of 6-chloro-4-cyclohexyl-1-isopropyl-1H-pyrazolo[3,4-d]pyrimidine (12a)

Compound 12a was prepared according to the procedure reported in step-1 of scheme 8, from 4,6-dichloro-1-isopropyl-1H-pyrazolo[3,4-d]pyrimidine (3a) (1 g, 4.33 mmol) in THF (20 mL) using cyclohexylmagnesium bromide (4.33 mL, 4.33 mmol) and copper (1) iodide (41 mg, 0.216 mmol). This gave after workup and purification using flash column chromatography [silica gel (12 g), eluting with EtOAc in hexane from 0-100%] 6-chloro-4-cyclohexyl-1-isopropyl-1H-pyrazolo[3,4-d]pyrimidine (12a) (726 mg, 60% yield); MS (ES+): 279.10 (M+1).

### Step-2: Preparation of 4-cyclohexyl-1-isopropyl-N-(1-(3,4,5-trimethoxyphenyl)-1H-imidazol-4-yl)-1H-pyrazolo[3,4-d]pyrimidin-6-amine (12b)

Compound 12b was prepared according to the procedure reported in step-4 of scheme 7, from 6-chloro-4-cyclohexyl-1-isopropyl-1H-pyrazolo[3,4-d]pyrimidine (12a) (1508 mg, 2.604 mmol) in toluene (20 mL) and t-butanol (5 mL) using 1-(3,4,5-trimethoxyphenyl)-1H-imidazol-4-amine (1b) (779 mg, 3.12 mmol) cesium carbonate (2970 mg, 9.11 mmol), Pd₂(dba)₃ (477 mg, 0.521 mmol), XPhos (497 mg, 1.042 mmol) and heating at 110° C. for 12 h under argon. This gave after workup and purification using flash column chromatography [silica gel (24 g), eluting with DMA-80 in DCM from 0-50%] followed by purification using reverse phase column chromatography [C18 column (50 g), eluting with ACN in water (containing 0.1% HCl) from 0-100%] 4-cyclohexyl-1-isopropyl-N-(1-(3,4,5-trimethoxyphenyl)-1H-imidazol-4-yl)-1H-pyrazolo[3,4-d]pyrimidin-6-amine (12b) (92 mg, 4% yield) HCl salt as a white solid; ¹H NMR (300 MHz, DMSO-d₆) δ 10.27 (s, 1H, D₂O exchangeable), 8.82 (s, 1H), 8.26 (s, 1H), 8.02 (s, 1H, D₂O exchangeable), 7.06 (s, 2H), 5.14-4.98 (m, 1H), 3.89 (s, 6H), 3.71 (s, 3H), 3.63-3.45 (m, 2H), 3.14-2.99 (m, 1H), 2.02-1.62 (m, 8H), 1.48 (d, J=6.7 Hz, 6H); MS (ES+): 492.3 (M+1).

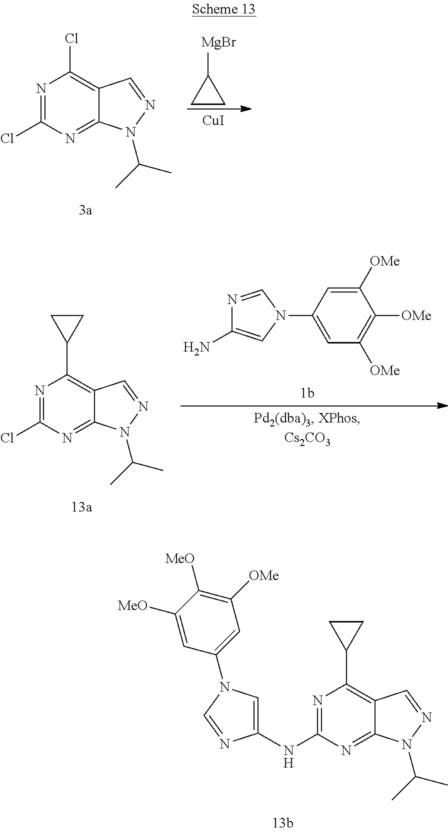

### Preparation of 4-cyclopropyl-1-isopropyl-N-(1-(3,4,5-trimethoxyphenyl)-1H-imidazol-4-yl)-1H-pyrazolo[3,4-d]pyrimidin-6-amine (13b)

### Step-1: Preparation of 6-chloro-4-cyclopropyl-1-isopropyl-1H-pyrazolo[3,4-d]pyrimidine (13a)

Compound 13a was prepared according to the procedure reported in step-1 of scheme 8, from 4,6-dichloro-1-isopropyl-1H-pyrazolo[3,4-d]pyrimidine (3a) (1 g, 4.33 mmol) in THF (20 mL) using cyclopropylmagnesium bromide (4.33 mL, 4.33 mmol) and copper (I) iodide (41 mg, 0.216 mmol). This gave after workup and purification using flash column chromatography [silica gel (12 g), eluting with EtOAc in hexane from 0-100%] 6-chloro-4-cyclopropyl-1-isopropyl-1H-pyrazolo[3,4-d]pyrimidine (13a) (122 mg, 12% yield); ¹H NMR (300 MHz, DMSO-d₆) δ 8.60 (s, 1H), 5.02 (hept. J=6.6 Hz, 1H), 2.71-2.60 (m, 1H), 1.47 (d, J=6.7 Hz, 6H), 1.32-1.23 (m, 4H); MS (ES+): 237.10 (M+1).

### Step-2: Preparation of 4-cyclopropyl-1-isopropyl-N-(1-(3,4,5-trimethoxyphenyl)-1H-imidazol-4-yl)-1H-pyrazolo[3,4-d]pyrimidin-6-amine (13b)

Compound 13b was prepared according to the procedure reported in step-4 of scheme 7, from 6-chloro-4-cyclopropyl-1-isopropyl-1H-pyrazolo[3,4-d]pyrimidine (13a) (299 mg, 0.5154 mmol) in toluene (20 mL) and t-butanol (5 mL) using 1-(3,4,5-trimethoxyphenyl)-1H-imidazol-4-amine (1b) (154 mg, 0.618 mmol) cesium carbonate (588 mg, 1.804 mmol), Pd₂(dba)₃ (94 mg, 0.103 mmol), XPhos (98 mg, 0.206 mmol) and heating at 110° C. for 12 h under argon. This gave after workup and purification using flash column chromatography [silica gel (24 g), eluting with DMA-80 in DCM from 0-50%] followed by purification using reverse phase column chromatography [C18 column (50 g), eluting with ACN in water (containing 0.1% HCl) from 0-100%] 4-cyclopropyl-1-isopropyl-N-(1-(3,4,5-trimethoxyphenyl)-1H-imidazol-4-yl)-1H-pyrazolo[3,4-d]pyrimidin-6-amine (13b) (57 mg, 14% yield) HCl salt as a light yellow solid; ¹H NMR (300 MHz, DMSO-d₆) δ 10.07 (s, 1H, D₂O exchangeable), 8.73 (s, 1H), 8.36-8.25 (m, 1H), 7.95 (s, 1H), 7.04 (d, J=3.8 Hz, 2H), 5.08-4.94 (m, 1H), 3.89 (s, 6H), 3.71 (s, 3H), 2.10-1.95 (m, 1H), 1.48 (d, J=6.7 Hz, 6H), 1.33-1.26 (m, 2H), 1.26-1.15 (m, 2H); MS (ES+): 450.2 (M+1).

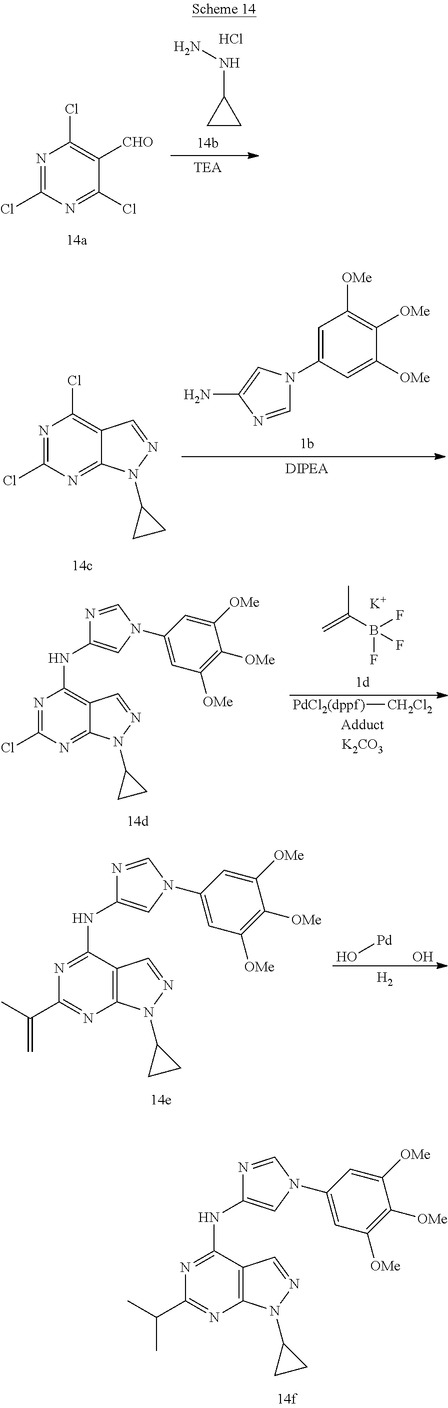

### Preparation of 1-cyclopropyl-6-isopropyl-N-(1-(3,4,5-trimethoxyphenyl)-1H-imidazol-4-yl)-1H-pyrazolo[3,4-d]pyrimidin-4-amine (14f)

### Step-1: Preparation of 4,6-dichloro-1-cyclopropyl-1H-pyrazolo[3,4-d]pyrimidine (14c)

To a solution of 2,4,6-trichloropyrimidine-5-carbaldehyde (14a) (1 g, 4.73 mmol; CAS #50270-27-4) in EtOH (10 mL) cooled to at −78° C. was added drop wise a solution of cyclopropyl hydrazine hydrochloride (14b) (0.513 g, 4.73 mmol; CAS #213764-25-1) in EtOH (10.00 mL), followed by triethylamine (TEA) (1.978 mL, 14.19 mmol) and stirred at −78° C. for 30 min. The reaction mixture was warmed to 0° C. over a period of 30 min, warmed to RT stirred for 2 h at RT and quenched with water (50 mL). The solid obtained was collected by filtration and purified using flash column chromatography [silica gel (24 g), eluting with EtOAc:MeOH (9:1) in hexane from 0-100%] to give 4,6-dichloro-1-cyclopropyl-1H-pyrazolo[3,4-d]pyrimidine (14c) (0.68 g, 63% yield) as a white solid; ¹H NMR (300 MHz, DMSO-d₆) δ 8.47 (d, J=2.4 Hz, 1H), 3.97-3.82 (m, 1H), 1.23-1.13 (m, 4H); MS (ES+): 229.00 & 231.00 (M+1).

### Step-2: Preparation of 6-chloro-1-cyclopropyl-N-(1-(3,4,5-trimethoxyphenyl)-1H-imidazol-4-yl)-1H-pyrazolo[3,4-d]pyrimidin-4-amine (14d)

Compound 14d was prepared according to the procedure reported in step-1 of scheme 1, from 4,6-dichloro-1-cyclopropyl-1H-pyrazolo[3,4-d]pyrimidine (14c) (0.6 g, 2.62 mmol) in 2-propanol (15 mL) using DIPEA (1.37 mL, 7.86 mmol), 1-(3,4,5-trimethoxyphenyl)-1H-imidazol-4-amine (1b) (0.653 g, 2.62 mmol) and heating at 90° C. for 4 h. This gave after work up 6-chloro-1-cyclopropyl-N-(1-(3,4,5-trimethoxyphenyl)-1H-imidazol-4-yl)-1H-pyrazolo[3,4-d]pyrimidin-4-amine (14d) (0.7 g 61% yield) as a white solid; ¹H NMR (300 MHz, DMSO-d₆) δ 11.34 (s, 1H), 8.38 (s, 1H), 8.17 (s, 1H), 7.87 (d, J=1.6 Hz, 1H), 6.92 (d, J=1.8 Hz, 2H), 3.87 (s, 6H), 3.85-3.75 (m, 1H), 3.70 (s, 3H), 1.22-1.03 (m, 4H); MS (ES+): 442.10 & 444.10 (M+1); (ES−): 440.10 & 442.10 (M−1).

### Step-3: Preparation of 1-cyclopropyl-6-(prop-1-en-2-yl)-N-(1-(3,4,5-trimethoxyphenyl)-1H-imidazol-4-yl)-1H-pyrazolo[3,4-d]pyrimidin-4-amine (14e)

Compound 14e was prepared according to the procedure reported in step-2 of scheme 1, from 6-chloro-1-cyclopropyl-N-(1-(3,4,5-trimethoxyphenyl)-1H-imidazol-4-yl)-1H-pyrazolo[3,4-d]pyrimidin-4-amine (14d) (420 mg, 0.951 mmol) in dioxane/H₂O (5 mL, ratio: 4:1) using potassium isopropenyltrifluoroborate (1d) (246 mg, 1.663 mmol), potassium carbonate (328 mg, 2.376 mmol), PdCl₂(dppf)-CH₂Cl₂ adduct (155 mg, 0.190 mmol) and beating for 1 h at 150° C. in a microwave. This gave after work up and purification by flash column chromatography [silica gel (24 g), eluting with DMA-80 in DCM from 0-80%] 1-cyclopropyl-6-(prop-1-en-2-yl)-N-(1-(3,4,5-trimethoxyphenyl)-1H-imidazol-4-yl)-1H-pyrazolo[3,4-d]pyrimidin-4-amine (14e) (0.2 g, 47% yield); ¹H NMR (300 MHz, DMSO-d₆) δ 10.88 (s, 1H), 8.36 (s, 1H), 8.23 (d, J=1.6 Hz, 1H), 8.10 (s, 1H), 6.94 (s, 2H), 6.48 (d, J=1.8 Hz, 1H), 5.56 (dd, J=2.8, 1.6 Hz, 1H), 3.97-3.89 (m, 1H), 3.87 (s, 6H), 3.69 (s, 3H), 2.31 (s, 3H), 1.26-1.15 (m, 2H), 1.14-1.03 (m, 2H); MS (ES+): 448.20 (M+1); (ES−): 446.15 (M−1).

### Step-4: Preparation of 1-cyclopropyl-6-isopropyl-N-(1-(3,4,5-trimethoxyphenyl)-1H-imidazol-4-yl)-1H-pyrazolo[3,4-d]pyrimidin-4-amine (14f)

Compound 14f was prepared according to the procedure reported in step-3 of scheme 1, from 1-cyclopropyl-6-(prop-1-en-2-yl)-N-(1-(3,4,5-trimethoxyphenyl)-1H-imidazol-4-yl)-1H-pyrazolo[3,4-d]pyrimidin-4-amine (14e) (200 mg, 0.447 mmol) in MeOH (10 mL) using palladium hydroxide on carbon, 20 wt. % loading (dry basis), matrix carbon, wet support (62.8 mg, 0.089 mmol) and stirring overnight at RT under a H₂ atmosphere. This gave after work up and purification using reverse phase column chromatography [C18 column (50 g), eluting with ACN in water (containing 0.1% HCl) from 0-100%] 1-cyclopropyl-6-isopropyl-N-(1-(3,4,5-trimethoxyphenyl)-1H-imidazol-4-yl)-1H-pyrazolo[3,4-d]pyrimidin-4-amine (14f) (112 mg, 56% yield) HCl salt as a white solid; ¹H NMR (300 MHz, DMSO-d₆) δ 11.79 (s, 1H), 8.55 (s, 1H), 8.40 (s, 1H), 8.09 (d, J=1.6 Hz, 1H), 7.00 (s, 2H), 3.96-3.95 (m, 1H), 3.88 (s, 6H), 3.70 (s, 3H), 3.19 (hept, J=6.8 Hz, 1H), 1.39 (d, J=6.8 Hz, 6H), 1.24-1.04 (m, 4H); MS (ES+): 450.2 (M+1); MS (ES−): 448.2 (M−1); Analysis calculated for: C₂₃H₂₇N₇O₃. 1HCl·1.25H₂O. C, 54.33; H, 6.05; Cl, 6.97; N, 19.28; Found: C, 54.53; H, 6.00; Cl, 6.65; N, 18.89.

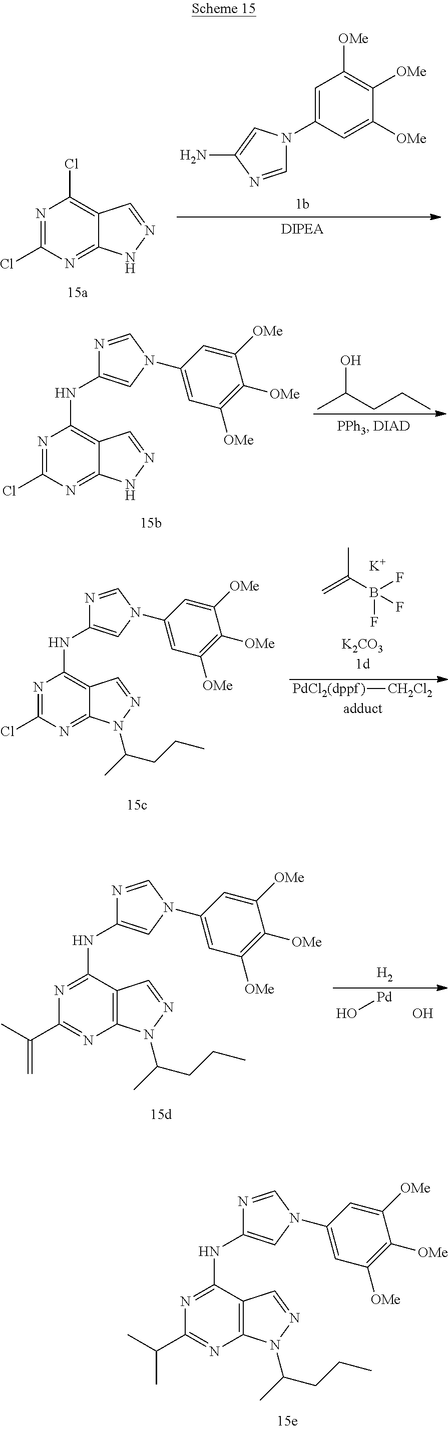

### Preparation of 6-isopropyl-1-(pentan-2-yl)-N-(1-(3,4,5-trimethoxyphenyl)-1H-imidazol-4-yl)-1H-pyrazolo[3,4-d]pyrimidin-4-amine (15e)

### Step-1: Preparation of 6-chloro-N-(1-(3,4,5-trimethoxyphenyl)-1H-imidazol-4-yl)-1H-pyrazolo[3,4-d]pyrimidin-4-amine (15b)

Compound 15b was prepared according to the procedure reported in step-1 of scheme 1, from 4,6-dichloro-1H-pyrazolo[3,4-d]pyrimidine (15a) (5 g, 26.5 mmol) in 2-propanol (150 mL) using DIPEA (13.86 mL, 79 mmol), 1-(3,4,5-trimethoxyphenyl)-1H-imidazol-4-amine (1b) (6.92 g, 27.8 mmol) and heating overnight at 90° C. This gave after work up 6-chloro-N-(1-(3,4,5-trimethoxyphenyl)-1H-imidazol-4-yl)-1H-pyrazolo[3,4-d]pyrimidin-4-amine (15b)(9.2 g, 87% yield) as an orange solid; MS (ES+): 402.10 & 404.05 (M+1); (ES−): 400.10 & 402.10 (M−1).

### Step-2: Preparation of 6-chloro-1-(pentan-2-yl)-N-(1-(3,4,5-trimethoxyphenyl)-1H-imidazol-4-yl)-1H-pyrazolo[3,4-d]pyrimidin-4-amine (15c)

To a mixture of triphenylphosphine (587 mg, 2.240 mmol), pentan-2-ol (197 mg, 2.240 mmol) and 6-chloro-N-(1-(3,4,5-trimethoxyphenyl)-1H-imidazol-4-yl)-1H-pyrazolo[3,4-d]pyrimidin-4-amine (15b) (500 mg, 1.244 mmol) in THF (10 mL) at 0° C. was added drop wise DIAD (0.436 mL, 2.240 mmol) and was stirred at 0° C. for 10 min. The reaction mixture was concentrated in vacuum and the residue obtained was purified by flash column chromatography [silica gel (24 g), eluting with DMA-80 in DCM from 0-60%] to give 6-chloro-1-(pentan-2-yl)-N-(1-(3,4,5-trimethoxyphenyl)-1H-imidazol-4-yl)-1H-pyrazolo[3,4-d]pyrimidin-4-amine (15c) (560 mg, 95% yield) as a yellow solid; MS (ES+): 472.10 & 474.10 (M+1); (ES−): 470.10 & 472.20 (M−1).

### Step-3: Preparation of 1-(pentan-2-yl)-6-(prop-1-en-2-yl)-N-(1-(3,4,5-trimethoxyphenyl)-1H-imidazol-4-yl)-1H-pyrazolo[3,4-d]pyrimidin-4-amine (15d)

Compound 15d was prepared according to the procedure reported in step-1 of scheme 1, from 6-chloro-1-(pentan-2-yl)-N-(1-(3,4,5-trimethoxyphenyl)-1H-imidazol-4-yl)-1H-pyrazolo[3,4-d]pyrimidin-4-amine (15c) (300 mg, 0.636 mmol) in dioxane/H₂O (5 mL, ratio: 4:1) using potassium isopropenyltrifluoroborate (l d) (165 mg, 1.112 mmol), potassium carbonate (220 mg, 1.589 mmol), PdCl₂(dppf)-CH₂Cl₂ adduct (104 mg, 0.127 mmol) and heating for 1 h at 150° C. in a microwave. This gave after work up and purification by flash column chromatography [silica gel (24 g), eluting with DMA-80 in DCM from 0-80%] 1-(pentan-2-yl)-6-(prop-1-en-2-yl)-N-(1-(3,4,5-trimethoxyphenyl)-1H-imidazol-4-yl)-1H-pyrazolo[3,4-d]pyrimidin-4-amine (15d) (80 mg, 26% yield); MS (ES+): 478.25 (M+1); (ES−): 476.20 (M−1).

### Step-4: Preparation of 6-isopropyl-1-(pentan-2-yl)-N-(1-(3,4,5-trimethoxyphenyl)-1H-imidazol-4-yl)-1H-pyrazolo[3,4-d]pyrimidin-4-amine (15e)

Compound 15e was prepared according to the procedure reported in step-3 of scheme 1, from 1-(pentan-2-yl)-6-(prop-1-en-2-yl)-N-(1-(3,4,5-trimethoxyphenyl)-1H-imidazol-4-yl)-1H-pyrazolo[3,4-d]pyrimidin-4-amine (15d) (80 mg, 0.168 mmol) in MeOH (10 mL) using palladium hydroxide on carbon, 20 wt. % loading (dry basis), matrix carbon, wet support (23.53 mg, 0.034 mmol) and stirring overnight at RT under a H₂ atmosphere. This gave after work up and purification using reverse phase column chromatography [C18 column (50 g), eluting with ACN in water (containing 0.1% HCl) from 0-100%] 6-isopropyl-1-(pentan-2-yl)-N-(1-(3,4,5-trimethoxyphenyl)-1H-imidazol-4-yl)-1H-pyrazolo[3,4-d]pyrimidin-4-amine (15e) (45 mg, 56% yield) HCl salt as a white solid; ¹H NMR (300 MHz, DMSO-d₆) δ 11.28 (s, 1H, D₂O exchangeable), 8.39 (s, 1H), 8.10 (d, J=1.7 Hz, 1H), 7.87-7.78 (m, 1H), 6.97 (s, 2H), 5.05-4.75 (m, 1H), 3.87 (s, 6H), 3.70 (s, 3H), 3.19-3.06 (m, 1H), 2.03-1.83 (m, 1H), 1.84-1.57 (m, 1H), 1.45 (d, J=6.7 Hz, 3H), 1.37 (d, J=6.8 Hz, 6H), 1.10-0.92 (m, 2H), 0.81 (t, J=7.2 Hz, 3H); MS (ES+): 480.2 (M+1); (ES−): 478.3 (M−1).

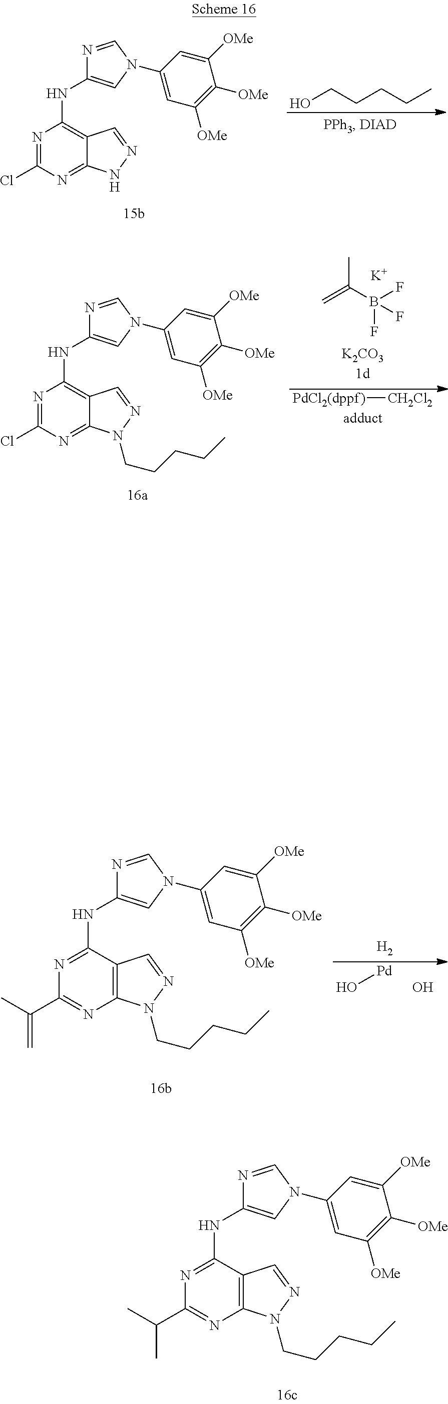

### Preparation of 6-isopropyl-1-pentyl-N-(1-(3,4,5-trimethoxyphenyl)-1H-imidazol-4-yl)-1H-pyrazolo[3,4-d]pyrimidin-4-amine (16c)

### Step-1: Preparation of 6-chloro-1-pentyl-N-(1-(3,4,5-trimethoxyphenyl)-1H-imidazol-4-yl)-1H-pyrazolo[3,4-d]pyrimidin-4-amine (16a)

Compound 16a was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (500 mg, 1.244 mmol) in THF (10 mL) using triphenylphosphine (587 mg, 2.240 mmol), pentan-1-ol (197 mg, 2.240 mmol), DIAD (0.436 mL, 2.240 mmol) and stirring at 0° C. for 10 min. This gave after work up and purification using flash column chromatography [silica gel (24 g), eluting with DMA-80 in DCM from 0-60%] to give 6-chloro-1-pentyl-N-(1-(3,4,5-trimethoxyphenyl)-1H-imidazol-4-yl)-1H-pyrazolo[3,4-d]pyrimidin-4-amine (16a) (580 mg, 99% yield) as a yellow solid; ¹H NMR (300 MHz, DMSO-d₆) δ 11.36 (s, 1H), 8.44 (s, 1H), 8.18 (s, 1H), 7.87 (d, J=1.6 Hz, 1H), 6.92 (s, 2H), 4.26 (t, J=7.0 Hz, 2H), 3.87 (s, 6H), 3.70 (s, 3H), 1.86-1.77 (m, 2H), 1.30 (d, J=7.4 Hz, 2H), 1.24-1.19 (m, 2H), 0.85-0.80 (m, 3H); MS (ES+): 472.20 (M+1); (ES−): 470.10 (M−1).

### Step-2: Preparation of 1-pentyl-6-(prop-1-en-2-yl)-N-(1-(3,4,5-trimethoxyphenyl)-1H-imidazol-4-yl)-1H-pyrazolo[3,4-d]pyrimidin-4-amine (16b)

Compound 16b was prepared according to the procedure reported in step-2 of scheme 1, from 6-chloro-1-pentyl-N-(1-(3,4,5-trimethoxyphenyl)-1H-imidazol-4-yl)-1H-pyrazolo[3,4-d]pyrimidin-4-amine (16a) (300 mg, 0.636 mmol) in dioxane/H₂O (5 mL, ratio: 4:1) using potassium isopropenyltrifluoroborate (1d)(165 mg, 1.112 mmol), potassium carbonate (220 mg, 1.589 mmol), PdCl₂(dppf)-CH₂Cl₂ adduct (104 mg, 0.127 mmol) and heating for 1 h at 150° C. in a microwave. This gave after work up and purification using flash column chromatography [silica gel (24 g), eluting with DMA-80 in DCM from 0-80%] 1-pentyl-6-(prop-1-en-2-yl)-N-(1-(3,4,5-trimethoxyphenyl)-1H-imidazol-4-yl)-1H-pyrazolo[3,4-d]pyrimidin-4-amine (16b) (80 mg, 26% yield); ¹H NMR (300 MHz, DMSO-d₆) δ 10.89 (s, 1H), 8.42 (s, 1H), 8.23 (d, J=1.6 Hz, 1H), 8.10 (s, 1H), 6.94 (s, 2H), 6.50-6.43 (m, 1H), 5.55 (dd, J=2.8, 1.6 Hz, 1H), 4.34 (t, J=6.8 Hz, 2H), 3.87 (s, 6H), 3.69 (s, 3H), 2.30 (s, 3H), 1.85 (p, J=6.9 Hz, 2H), 1.38-1.25 (m, 2H), 1.25-1.13 (m, 2H), 0.82 (t, J=7.2 Hz, 3H); MS (ES+): 478.20 (M+1).

### Step-3: Preparation of 6-isopropyl-1-pentyl-N-(1-(3,4,5-trimethoxyphenyl)-1H-imidazol-4-yl)-1H-pyrazolo[3,4-d]pyrimidin-4-amine (16c)

Compound 16c was prepared according to the procedure reported in step-3 of scheme 1, from 1-pentyl-6-(prop-1-en-2-yl)-N-(1-(3,4,5-trimethoxyphenyl)-1H-imidazol-4-yl)-1H-pyrazolo[3,4-d]pyrimidin-4-amine (16b) (80 mg, 0.168 mmol) in MeOH (10 mL) using palladium hydroxide on carbon, 20 wt. % loading (dry basis), matrix carbon, wet support (23.53 mg, 0.034 mmol) and stirring overnight at RT under a H₂ atmosphere. This gave after work up and purification using reverse phase column chromatography [C18 column (50 g), eluting with ACN in water (containing 0.1% HCl) from 0-100%] 6-isopropyl-1-pentyl-N-(1-(3,4,5-trimethoxyphenyl)-1H-imidazol-4-yl)-1H-pyrazolo[3,4-d]pyrimidin-4-amine (16c) (45 mg, 56% yield) HCl salt as a white solid; ¹H NMR (300 MHz, DMSO-d₆) δ 11.53 (s, 1H), 8.47 (m, 2H), 8.10 (d, J=1.6 Hz, 1H), 6.98 (s, 2H), 4.34 (t, J=6.9 Hz, 2H), 3.88 (s, 6H), 3.70 (s, 3H), 3.16 (p, J=6.8 Hz, 1H), 1.83 (p, J=7.1 Hz, 2H), 1.37 (d, J=6.9 Hz, 6H), 1.34-1.26 (m, 2H), 1.24-1.14 (m, 2H), 0.83 (t, J=7.2 Hz, 3H); MS (ES+): 480.3 (M+1); MS (ES−): 478.2 (M−1).

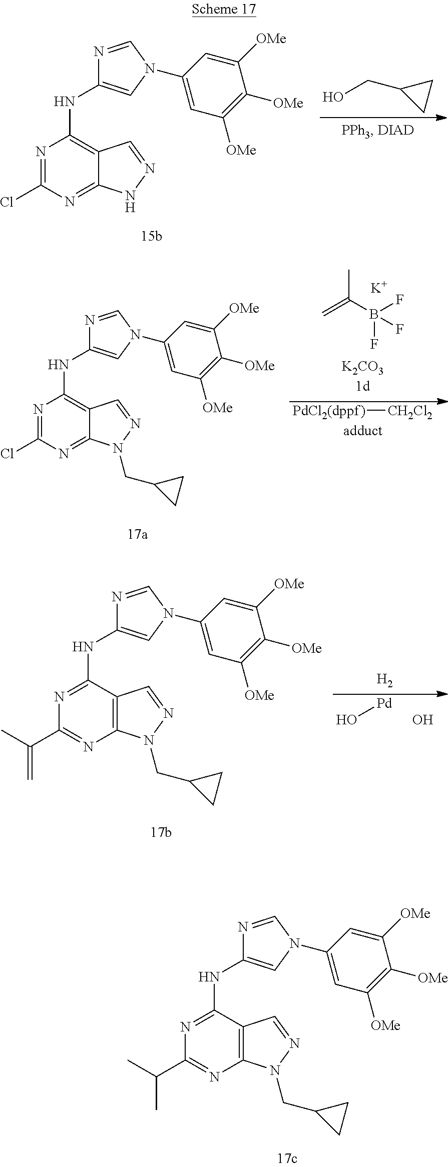

### Preparation of 1-(cyclopropylmethyl)-6-isopropyl-N-(1-(3,4,5-trimethoxyphenyl)-1H-imidazol-4-yl)-1H-pyrazolo[3,4-d]pyrimidin-4-amine (17c)

### Step-1: Preparation of 6-chloro-1-(cyclopropylmethyl)-N-(1-(3,4,5-trimethoxyphenyl)-1H-imidazol-4-yl)-1H-pyrazolo[3,4-d]pyrimidin-4-amine (17a)

Compound 17a was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (4 g, 9.96 mmol) in THF (100 mL) using triphenylphosphine (4.70 g, 17.92 mmol), cyclopropylmethanol (1.292 g, 17.92 mmol), DIAD (3.62 g, 17.92 mmol) and stirring at 0° C. for 5 min. This gave after work up and purification using flash column chromatography [silica gel (80 g), eluting with DMA-80 in DCM from 0-60%] 6-chloro-1-(cyclopropylmethyl)-N-(1-(3,4,5-trimethoxyphenyl)-1H-imidazol-4-yl)-1H-pyrazolo[3,4-d]pyrimidin-4-amine (17a) (2.5 g, 55% yield) as a yellow solid; MS (ES+): 456.15 & 458.10 (M+1); (ES−): 454.10 & 456.10 (M−1).

### Step-2: Preparation of 1-(cyclopropylmethyl)-6-(prop-1-en-2-yl)-N-(1-(3,4,5-trimethoxyphenyl)-1H-imidazol-4-yl)-1H-pyrazolo[3,4-d]pyrimidin-4-amine (17b)

Compound 17b was prepared according to the procedure reported in step-2 of scheme 1, from 6-chloro-1-(cyclopropylmethyl)-N-(1-(3,4,5-trimethoxyphenyl)-1H-imidazol-4-yl)-1H-pyrazolo[3,4-d]pyrimidin-4-amine (17a) (550 mg, 1.206 mmol) in dioxane/H₂O (10 mL, ratio: 4:1) using potassium isopropenyltrifluoroborate (1d)(312 mg, 2.111 mmol), potassium carbonate (417 mg, 3.02 mmol), PdCl₂(dppf)-CH₂Cl₂ adduct (197 mg, 0.241 mmol) and stirring for 1 h at 150° C. on microwave. This gave after work up and purification using flash column chromatography [silica gel (24 g), eluting with DMA-80 in DCM from 0-80%] 1-(cyclopropylmethyl)-6-(prop-1-en-2-yl)-N-(1-(3,4,5-trimethoxyphenyl)-1H-imidazol-4-yl)-1H-pyrazolo[3,4-d]pyrimidin-4-amine (17b) (80 mg, 26% yield); MS (ES+): 462.20 (M+1); (ES−): 460.15 (M−1).

### Step-3: Preparation of 1-(cyclopropylmethyl)-6-isopropyl-N-(1-(3,4,5-trimethoxyphenyl)-1H-imidazol-4-yl)-1H-pyrazolo[3,4-d]pyrimidin-4-amine (17c)

Compound 17c was prepared according to the procedure reported in step-3 of scheme 1, from 1-(cyclopropylmethyl)-6-(prop-1-en-2-yl)-N-(1-(3,4,5-trimethoxyphenyl)-1H-imidazol-4-yl)-1H-pyrazolo[3,4-d]pyrimidin-4-amine (17b) (160 mg, 0.347 mmol) in MeOH (10 mL) using palladium hydroxide on carbon, 20 wt. % loading (dry basis), matrix carbon, wet support (48.7 mg, 0.069 mmol) and stirring overnight at RT under a H₂ atmosphere. This gave after work up and purification using reverse phase column chromatography [C18 column (50 g), eluting with ACN in water (containing 0.1% HCl) from 0-100%] 1-(cyclopropylmethyl)-6-isopropyl-N-(1-(3,4,5-trimethoxyphenyl)-1H-imidazol-4-yl)-1H-pyrazolo[3,4-d]pyrimidin-4-amine (17c) (95 mg, 59% yield) HCl salt as a white solid; ¹H NMR (300 MHz, DMSO-d₆) δ 12.09 (s, 1H, D₂O exchangeable), 8.61 (s, 1H), 8.52 (s, 1H), 8.08 (d, J=1.5 Hz, 1H), 7.02 (s, 2H), 4.25 (d, J=7.1 Hz, 2H), 3.88 (s, 6H), 3.70 (s, 3H), 3.20 (hept, J=6.7 Hz, 1H), 1.38 (dd, J=6.8, 1.1 Hz, 6H), 1.34-1.18 (m, 1H), 0.57-0.37 (m, 4H); MS (ES+): 464.20 (M+1); Analysis calculated for C₂₄H₂₉N₇O₃·HCl·2H₂O: C, 53.78; H, 6.39; Cl, 6.61; N, 18.29; Found: C, 53.76; H, 6.33; Cl, 6.38; N, 18.11.

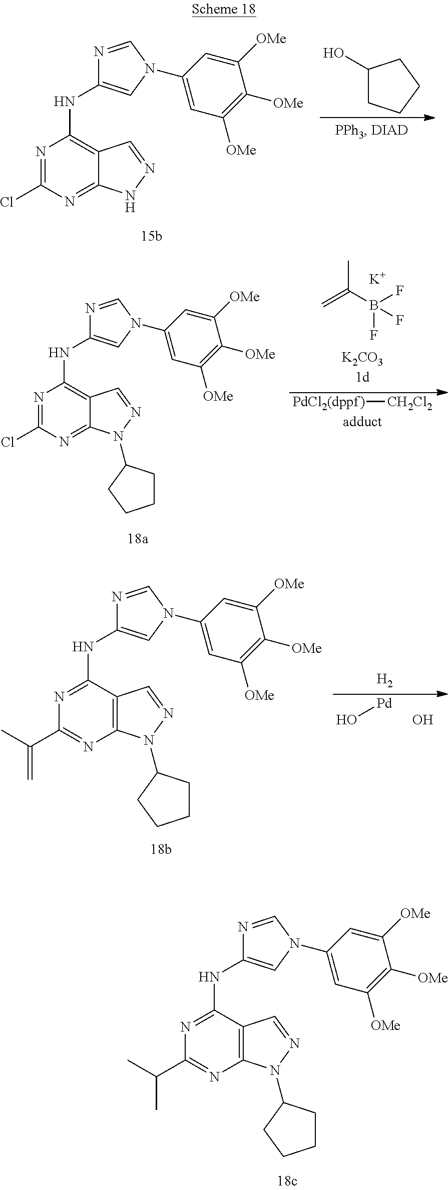

### Preparation of 1-cyclopentyl-6-isopropyl-N-(1-(3,4,5-trimethoxyphenyl)-1H-imidazol-4-yl)-1H-pyrazolo[3,4-d]pyrimidin-4-amine (18c)

### Step-1: Preparation of 6-chloro-1-cyclopentyl-N-(1-(3,4,5-trimethoxyphenyl)-1H-imidazol-4-yl)-1H-pyrazolo[3,4-d]pyrimidin-4-amine (18a)

Compound 18a was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (500 mg, 1.244 mmol) in THF (20 mL) using triphenylphosphine (587 mg, 2.240 mmol), cyclopentanol (193 mg, 2.240 mmol), DIAD (0.436 mL, 2.240 mmol) and stirring at 0° C. for 5 min. This gave after work up and purification using flash column chromatography [silica gel (24 g), eluting with DMA-80 in DCM from 0-60%] 6-chloro-1-cyclopentyl-N-(1-(3,4,5-trimethoxyphenyl)-1H-imidazol-4-yl)-1H-pyrazolo[3,4-d]pyrimidin-4-amine (18a) (210 mg, 36% yield) as a brown solid; MS (ES+): 470.10 & 472.10 (M+1); (ES−): 468.15 & 470.20 (M−1).

### Step-2: Preparation of 1-cyclopentyl-6-(prop-1-en-2-yl)-N-(1-(3,4,5-trimethoxyphenyl)-1H-imidazol-4-yl)-1H-pyrazolo[3,4-d]pyrimidin-4-amine (18b)

Compound 18b was prepared according to the procedure reported in step-2 of scheme 1, from 6-chloro-1-cyclopentyl-N-(1-(3,4,5-trimethoxyphenyl)-1H-imidazol-4-yl)-1H-pyrazolo[3,4-d]pyrimidin-4-amine (18a) (210 mg, 0.447 mmol) in dioxane/H₂O (8 mL, ratio: 4:1) using potassium isopropenyltrifluoroborate (1d) (116 mg, 0.782 mmol), potassium carbonate (154 mg, 1.117 mmol), PdCl₂(dppf)-CH₂Cl₂ adduct (73.0 mg, 0.089 mmol) and beating for 1 h at 150° C. in a microwave. This gave after work up and purification using flash column chromatography [silica gel (24 g), eluting with DMA-80 in DCM from 0-80%] 1-cyclopentyl-6-(prop-1-en-2-yl)-N-(1-(3,4,5-trimethoxyphenyl)-1H-imidazol-4-yl)-1H-pyrazolo[3,4-d]pyrimidin-4-amine (18b) (125 mg, 59% yield); ¹H NMR (300 MHz, DMSO-d₆) δ 10.88 (s, 1H), 8.42 (s, 1H), 8.23 (d, J=1.6 Hz, 1H), 8.10 (s, 1H), 6.94 (s, 2H), 6.52-6.42 (m, 1H), 5.55 (dd, J=2.8, 1.6 Hz, 1H), 5.25 (p, J=7.2 Hz, 1H), 3.88 (s, 6H), 3.70 (s, 3H), 2.30 (s, 3H), 2.17-1.62 (m, 8H); MS (ES+): 476.20 (M+1).

### Step-3: Preparation of 1-cyclopentyl-6-isopropyl-N-(1-(3,4,5-trimethoxyphenyl)-1H-imidazol-4-yl)-1H-pyrazolo[3,4-d]pyrimidin-4-amine (18c)

Compound 18c was prepared according to the procedure reported in step-3 of scheme 1, from 1-cyclopentyl-6-(prop-1-en-2-yl)-N-(1-(3,4,5-trimethoxyphenyl)-1H-imidazol-4-yl)-1H-pyrazolo[3,4-d]pyrimidin-4-amine (18b) (120 mg, 0.252 mmol) in MeOH (10 mL) using palladium hydroxide on carbon, 20 wt. % loading (dry basis), matrix carbon, wet support (35.4 mg, 0.050 mmol) and stirring overnight at RT under a H₂ atmosphere. This gave after work up and purification using reverse phase column chromatography [C18 column (50 g), eluting with ACN in water (containing 0.1% HCl) from 0-100%] 1-cyclopentyl-6-isopropyl-N-(1-(3,4,5-trimethoxyphenyl)-1H-imidazol-4-yl)-1H-pyrazolo[3,4-d]pyrimidin-4-amine (18c) (56 mg, 47% yield) HCl salt as a white solid; ¹H NMR (300 MHz, DMSO-d₆) δ 12.02 (s, 1H, D₂O exchangeable), 8.58 (s, 1H), 8.51 (s, 1H), 8.07 (d, J=1.5 Hz, 1H, D₂O exchangeable), 7.01 (s, 2H), 5.27 (p, J=7.1 Hz, 1H), 3.88 (s, 6H), 3.70 (s, 3H), 3.19 (hept,J=6.8 Hz, 1H), 2.18-1.65 (m, 8H), 1.38 (d, J=6.8 Hz, 6H); MS (ES+): 478.20 (M+1); Analysis calculated for C₂₃H₃₁N₇O₃·HCl·H₂O: C, 56.44; H, 6.44; Cl, 6.66; N, 18.43; Found: C, 56.26; H, 6.20; Cl, 6.28; N, 18.06.

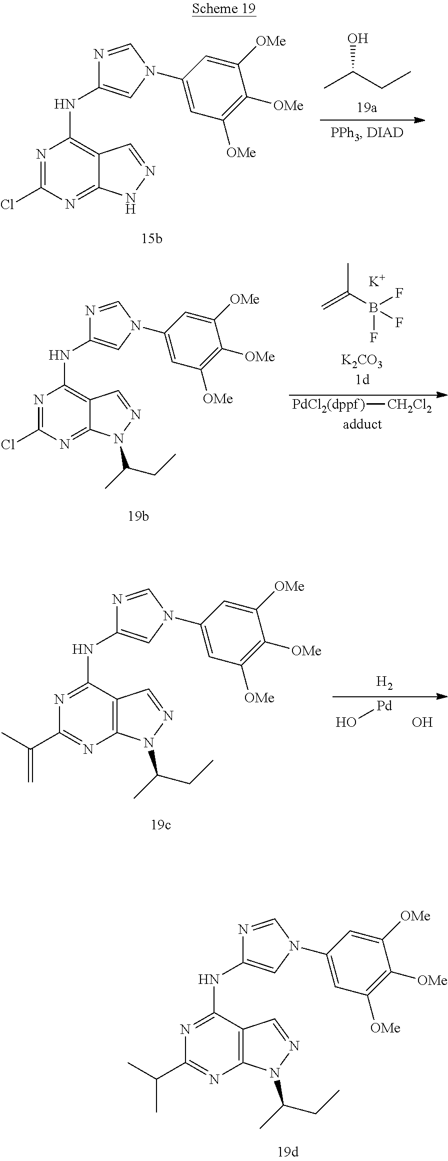

### Preparation of (R)-1-(sec-butyl)-6-isopropyl-N-(1-(3,4,5-trimethoxyphenyl)-1H-imidazol-4-yl)-1H-pyrazolo[3,4-d]pyrimidin-4-amine (19d)

### Step-1: Preparation of (R)-1-(sec-butyl)-6-chloro-N-(1-(3,4,5-trimethoxyphenyl)-1H-imidazol-4-yl)-1H-pyrazolo[3,4-d]pyrimidin-4-amine (19b)

Compound 19b was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (0.6 g, 1.493 mmol) in THF (20 mL) using triphenylphosphine (0.705 g, 2.69 mmol), (s)-butan-2-ol (19a) (0.199 g 2.69 mmol; CAS #4221-99-2), DIAD (0.523 mL, 2.69 mmol) and stirring at 0° C. for 10 min. This gave after work up and purification using flash column chromatography [silica gel (24 g), eluting with DMA80 in DCM from 0-60%] (R)-1-(sec-butyl)-6-chloro-N-(1-(3,4,5-trimethoxyphenyl)-1H-imidazol-4-yl)-1H-pyrazolo[3,4-d]pyrimidin-4-amine (19b) (0.24 g, 35% yield) as a yellow solid; MS (ES+): 458.10 & 460.10 (M+1); (ES−): 456.10 & 458.10 (M−1).

### Step-2: Preparation of (R)-1-(sec-butyl)-6-(prop-1-en-2-yl)-N-(1-(3,4,5-trimethoxyphenyl)-1H-imidazol-4-yl)-1H-pyrazolo[3,4-d]pyrimidin-4-amine (19c)

Compound 19c was prepared according to the procedure reported in step-2 of scheme 1, from (R)-1-(sec-butyl)-6-chloro-N-(1-(3,4,5-trimethoxyphenyl)-1H-imidazol-4-yl)-1H-pyrazolo[3,4-d]pyrimidin-4-amine (19b) (240 mg, 0.524 mmol) in dioxane/H₂O (8 mL, ratio: 4:1) using potassium isopropenyltrifluoroborate (1d)(136 mg, 0.917 mmol), potassium carbonate (181 mg, 1.310 mmol), PdCl₂(dppf)-CH₂Cl₂ adduct (86 mg, 0.105 mmol) and heating for 1 h at 150° C. in a microwave. This gave after work up and purification using flash column chromatography [silica gel (24 g), eluting with DMA-80 in DCM from 0-80%] (R)-1-(sec-butyl)-6-(prop-1-en-2-yl)-N-(1-(3,4,5-trimethoxyphenyl)-1H-imidazol-4-yl)-1H-pyrazolo[3,4-d]pyrimidin-4-amine (19c) (100 mg, 41% yield) as a white solid; MS (ES+): 464.20 (M+1).

### Step-3: Preparation of (R)-1-(sec-butyl)-6-isopropyl-N-(1-(3,4,5-trimethoxyphenyl)-1H-imidazol-4-yl)-1H-pyrazolo[3,4-d]pyrimidin-4-amine (19d)

Compound 19d was prepared according to the procedure reported in step-3 of scheme 1, from (R)-1-(sec-butyl)-6-(prop-1-en-2-yl)-N-(1-(3,4,5-trimethoxyphenyl)-1H-imidazol-4-yl)-1H-pyrazolo[3,4-d]pyrimidin-4-amine (19c) (100 mg, 0.216 mmol) in MeOH (10 mL) using palladium hydroxide on carbon, 20 wt. % loading (dry basis), matrix carbon, wet support (30.3 mg, 0.043 mmol) and stirring for 2 days at RT under a H₂ atmosphere. This gave after work up and purification using reverse phase column chromatography [C18 column (50 g), eluting with ACN in water (containing 0.1% HCl) from 0-100%] (R)-1-(sec-butyl)-6-isopropyl-N-(1-(3,4,5-trimethoxyphenyl)-1H-imidazol-4-yl)-1H-pyrazolo[3,4-d]pyrimidin-4-amine (19d) (38 mg, 38% yield) HCl salt as a white solid; ¹H NMR (300 MHz, DMSO-d₆) δ 11.53 (s, 1H), 8.54-8.37 (m, 2H), 8.10 (s, J=1.6 Hz, 1H), 6.98 (s, 2H), 4.92-4.76 (m, 1H), 3.88 (s, 6H), 3.70 (s, 3H), 3.24-3.08 (m, 1H), 2.00-1.75 (m, 2H), 1.46 (d, J=6.6 Hz, 3H), 1.37 (d, J=6.8 Hz, 6H), 0.67 (t, J=7.3 Hz, 3H); MS (ES+): 466.20 (M+1); Chiral HPLC: AD-H column 70/30 [(0.1% DEA in Hexane in 0.1% DEA in ethanol)] 1.0 mL/min UV detection 256 nm, 30 mins run time (Temp 40° C.); compound 19d [Rₜ=13.75 (peak-1), 93.50%]; compound 22d [Rₜ=22.59 (peak-2), 6.50%]; 86.99% ee; Optical rotation: [α]D=(−) 11.429 [CH₃OH, 0.14].

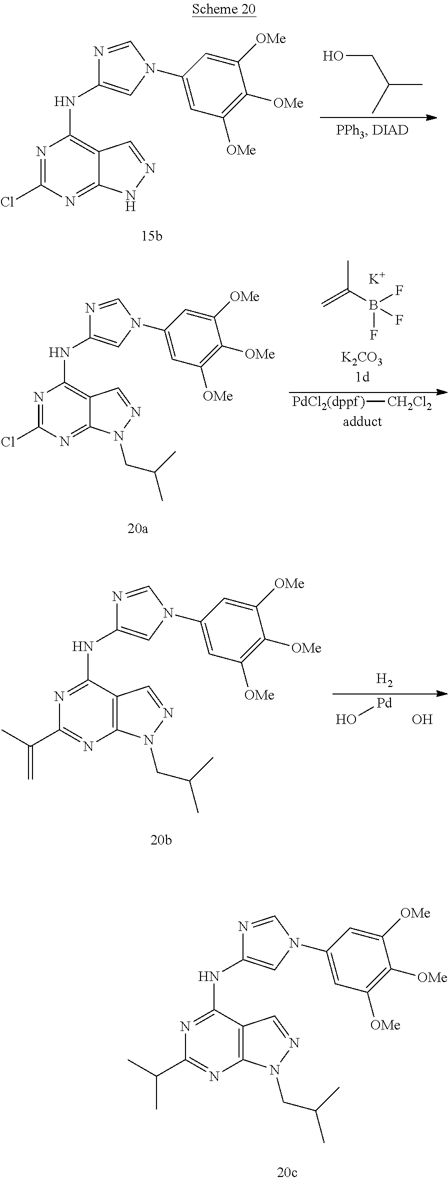

### Preparation of 1-isobutyl-6-isopropyl-N-(1-(3,4,5-trimethoxyphenyl)-1H-imidazol-4-yl)-1H-pyrazolo[3,4-d]pyrimidin-4-amine (20c)

### Step-1: Preparation of 6-chloro-1-isobutyl-N-(1-(3,4,5-trimethoxyphenyl)-1H-imidazol-4-yl)-1H-pyrazolo[3,4-d]pyrimidin-4-amine (20a)

Compound 20a was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (5 g, 12.44 mmol) in THF (100 mL) using triphenylphosphine (5.87 g, 22.40 mmol), 2-methylpropan-1-ol (1.660 g, 22.40 mmol), DIAD (4.36 mL, 22.40 mmol) and stirring at 0° C. for 10 min. This gave after work up and purification using flash column chromatography [silica gel (24 g), eluting with EtOAc in hexane from 0-60%] 6-chloro-1-isobutyl-N-(1-(3,4,5-trimethoxyphenyl)-1H-imidazol-4-yl)-1H-pyrazolo[3,4-d]pyrimidin-4-amine (20a) (2.5 g, 44% yield) as a clear oil; MS (ES+): 458.10 & 460.15 (M+1); (ES−): 456.15 & 458.15 (M−1).

### Step-2: Preparation of 1-isobutyl-6-(prop-1-en-2-yl)-N-(1-(3,4,5-trimethoxyphenyl)-1H-imidazol-4-yl)-1H-pyrazolo[3,4-d]pyrimidin-4-amine (20b)

Compound 20b was prepared according to the procedure reported in step-2 of scheme 1, from 6-chloro-1-isobutyl-N-(1-(3,4,5-trimethoxyphenyl)-1H-imidazol-4-yl)-1H-pyrazolo[3,4-d]pyrimidin-4-amine (20a) (150 mg, 0.328 mmol) in dioxane/H₂O (8 mL, ratio: 4:1) using potassium isopropenyltrifluoroborate (1d)(85 mg, 0.573 mmol), potassium carbonate (113 mg, 0.819 mmol), PdCl₂(dppf)-CH₂Cl₂ adduct (53.5 mg, 0.066 mmol) and heating for 1 h at 150° C. in a microwave. This gave after work up and purification using flash column chromatography [silica gel (24 g), eluting with DMA-80 in DCM from 0-80%] 1-isobutyl-6-(prop-1-en-2-yl)-N-(1-(3,4,5-trimethoxyphenyl)-1H-imidazol-4-yl)-1H-pyrazolo[3,4-d]pyrimidin-4-amine (20b) (100 mg, 66% yield) as a white solid; MS (ES+): 464.20 (M+1).

### Step-3: Preparation of 1-isobutyl-6-isopropyl-N-(1-(3,4,5-trimethoxyphenyl)-1H-imidazol-4-yl)-1H-pyrazolo[3,4-d]pyrimidin-4-amine (20c)

Compound 20c was prepared according to the procedure reported in step-3 of scheme 1, from 1-isobutyl-6-(prop-1-en-2-yl)-N-(1-(3,4,5-trimethoxyphenyl)-1H-imidazol-4-yl)-1H-pyrazolo[3,4-d]pyrimidin-4-amine (20b) (100 mg, 0.216 mmol) in MeOH (10 mL) using palladium hydroxide on carbon, 20 wt. % loading (dry basis), matrix carbon, wet support (30.3 mg, 0.043 mmol) and stirring for 2 days at RT under a H₂ atmosphere. This gave after work up and purification using reverse phase column chromatography [C18 column (50 g), eluting with ACN in water (containing 0.1% HCl) from 0-100%] 1-isobutyl-6-isopropyl-N-(1-(3,4,5-trimethoxyphenyl)-1H-imidazol-4-yl)-1H-pyrazolo[3,4-d]pyrimidin-4-amine (20c) (24 mg, 24% yield) HCl salt as a white solid; ¹H NMR (300 MHz, DMSO-d₆) δ 11.16 (s, 1H), 8.47-8.25 (m, 2H), 8.12 (s, 1H), 6.96 (s, 2H), 4.14 (d, J=7.1 Hz, 2H), 3.87 (s, 6H), 3.70 (s, 3H), 3.12 (p, J=6.8 Hz, 1H), 2.31-2.15 (m, 1H), 1.37 (d, J=6.9 Hz, 6H), 0.85 (d, J=6.7 Hz, 6H); MS (ES+): 466.20 (M+1).

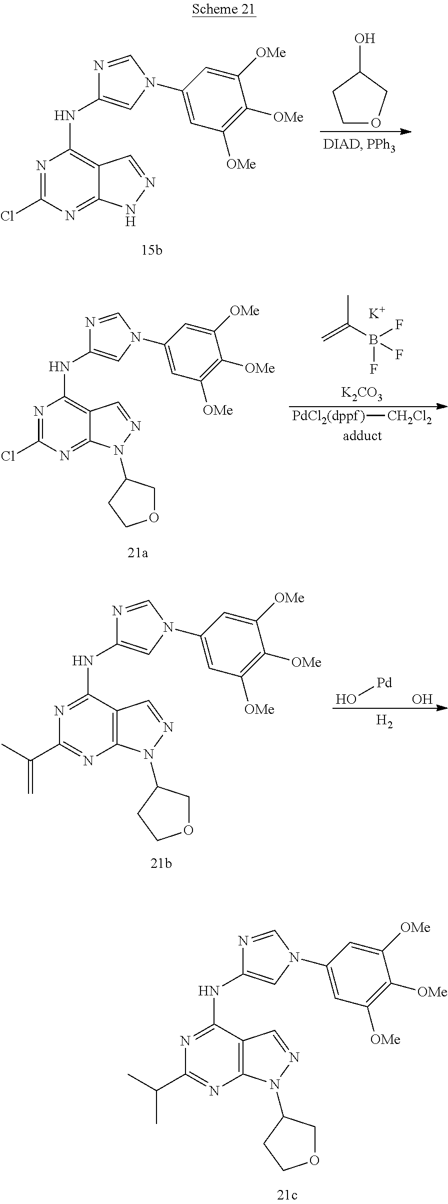

### Preparation of 6-isopropyl-1-(tetrahydrofuran-3-yl)-N-(1-(3,4,5-trimethoxyphenyl)-1H-imidazol-4-yl)-1H-pyrazolo[3,4-d]pyrimidin-4-amine (21c)

### Step-1: Preparation of 6-chloro-1-(tetrahydrofuran-3-yl)-N-(1-(3,4,5-trimethoxyphenyl)-1H-imidazol-4-yl)-1H-pyrazolo[3,4-d]pyrimidin-4-amine (21a)

Compound 21a was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (1 g, 2.489 mmol) in THF (20 mL) using triphenylphosphine (1.175 g, 4.48 mmol), tetrahydrofuran-3-ol (0.395 g, 4.48 mmol; CAS #453-20-3), DIAD (0.871 mL, 4.48 mmol) and stirring at 0° C. for 10 min. This gave after work up and purification using flash column chromatography [silica gel (24 g), eluting with DMA-80 in DCM from 0-60%] to give 6-chloro-1-(tetrahydrofuran-3-yl)-N-(1-(3,4,5-trimethoxyphenyl)-1H-imidazol-4-yl)-1H-pyrazolo[3,4-d]pyrimidin-4-amine (21a) (0.26 g, 22% yield) as a yellow solid; MS (ES+): 472.15 (M+1); (ES−): 470.15 (M−1).

### Step-2: Preparation of 6-(prop-1-en-2-yl)-1-(tetrahydrofuran-3-yl)-N-(1-(3,4,5-trimethoxyphenyl)-1H-imidazol-4-yl)-1H-pyrazolo[3,4-d]pyrimidin-4-amine (21b)

Compound 21b was prepared according to the procedure reported in step-2 of scheme 1, from 6-chloro-1-(tetrahydrofuran-3-yl)-N-(1-(3,4,5-trimethoxyphenyl)-1H-imidazol-4-yl)-1H-pyrazolo[3,4-d]pyrimidin-4-amine (21a) (0.26 g, 0.551 mmol) in dioxane/H₂O (8 mL, ratio: 4:1) using potassium isopropenyltrifluoroborate (1d)(143 mg, 0.964 mmol), potassium carbonate (190 mg, 1.377 mmol), PdCl₂(dppf)-CH₂Cl₂ adduct (90 mg, 0.110 mmol) and heating for 1 h at 150° C. in a microwave. This gave after work up and purification using flash column chromatography [silica gel (24 g), eluting with DMA-80 in DCM from 0-80%] 6-(prop-1-en-2-yl)-1-(tetrahydrofuran-3-yl)-N-(1-(3,4,5-trimethoxyphenyl)-1H-imidazol-4-yl)-1H-pyrazolo[3,4-d]pyrimidin-4-amine (21b) (0.12 g, 46% yield) as a white solid; MS (ES+): 478.20 (M+1); (ES−): 476.20 (M−1).

### Step-3: Preparation of 6-isopropyl-1-(tetrahydrofuran-3-yl)-N-(1-(3,4,5-trimethoxyphenyl)-1H-imidazol-4-yl)-1H-pyrazolo[3,4-d]pyrimidin-4-amine (21c)

Compound 21c was prepared according to the procedure reported in step-3 of scheme 1, from 6-(prop-1-en-2-yl)-1-(tetrahydrofuran-3-yl)-N-(1-(3,4,5-trimethoxyphenyl)-1H-imidazol-4-yl)-1H-pyrazolo[3,4-d]pyrimidin-4-amine (21b) (120 mg, 0.251 mmol) in MeOH (10 mL) using palladium hydroxide on carbon, 20 wt. % loading (dry basis), matrix carbon, wet support (35.3 mg, 0.050 mmol) and stirring for 2 days at RT under a H₂ atmosphere. This gave after work up and purification using flash column chromatography [silica gel (12 g), eluting with DMA-80 in DCM from 0-50%] followed by purification using reverse phase column chromatography [C18 column (50 g), eluting with ACN in water (containing 0.1% HCl) from 0-100%] 6-isopropyl-1-(tetrahydrofuran-3-yl)-N-(1-(3,4,5-trimethoxyphenyl)-1H-imidazol-4-yl)-1H-pyrazolo[3,4-d]pyrimidin-4-amine (21c) (38 mg, 32% yield) HCl salt as a white solid; ¹H NMR (300 MHz, DMSO-d₆) δ 11.16 (s, 1H), 8.58-8.26 (m, 2H), 8.12 (d, J=1.6 Hz, 1H), 6.96 (s, 2H), 5.56-5.40 (m, 1H), 4.20-4.00 (m, 2H), 3.99-3.85 (m, 8H), 3.70 (s, 3H), 3.22-3.03 (m, 1H), 2.46-2.21 (m, 2H), 1.37 (d, J=6.9 Hz, 6H); MS (ES+): 480.20 (M+1).

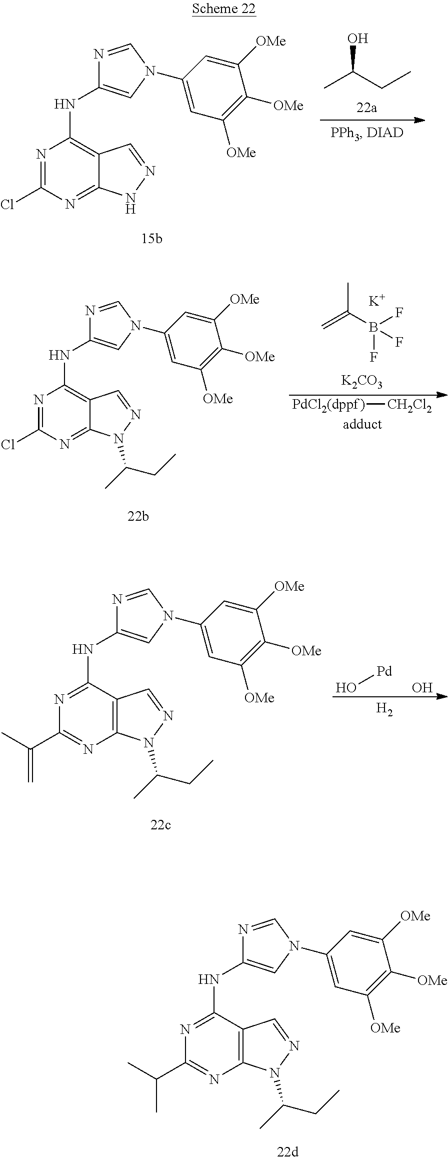

### Preparation of (S)-1-(sec-butyl)-6-isopropyl-N-(1-(3,4,5-trimethoxyphenyl)-1H-imidazol-4-yl)-1H-pyrazolo[3,4-d]pyrimidin-4-amine (22d)

### Step-1: Preparation of (S)-1-(sec-butyl)-6-chloro-N-(1-(3,4,5-trimethoxyphenyl)-1H-imidazol-4-yl)-1H-pyrazolo[3,4-d]pyrimidin-4-amine (22b)

Compound 22b was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (2.5 g, 6.22 mmol) in THF (40 mL) using triphenylphosphine, (R)-butan-2-ol (22a), DIAD and stirring at 0° C. for 10 min. This gave after work up and purification using flash column chromatography [silica gel (80 g), eluting with DMA-80 in DCM from 0-60%] (S)-1-(sec-butyl)-6-chloro-N-(1-(3,4,5-trimethoxyphenyl)-1H-imidazol-4-yl)-1H-pyrazolo[3,4-d]pyrimidin-4-amine (22b) (0.6 g, 21% yield) as a yellow solid; ¹H NMR (300 MHz, DMSO-d₆) δ 11.36 (s, 1H), 8.47 (s, 1H), 8.18 (s, 1H), 7.87 (d, J=1.6 Hz, 1H), 6.92 (s, 2H), 4.70 (q, J=6.8 Hz, 1H), 3.87 (s, 6H), 3.69 (s, 3H), 1.97-1.74 (m, 2H), 1.44 (d, J=6.7 Hz, 3H), 0.67 (t, J=7.3 Hz, 3H); MS (ES+): 458.10 (M+1); (ES−): 456.10 (M−1).

### Step-2: Preparation of (S)-1-(sec-butyl)-6-(prop-1-en-2-yl)-N-(1-(3,4,5-trimethoxyphenyl)-1H-imidazol-4-yl)-1H-pyrazolo[3,4-d]pyrimidin-4-amine (22c)

Compound 22c was prepared according to the procedure reported in step-2 of scheme 1, from (S)-1-(sec-butyl)-6-chloro-N-(1-(3,4,5-trimethoxyphenyl)-1H-imidazol-4-yl)-1H-pyrazolo[3,4-d]pyrimidin-4-amine (22b) (0.6 g, 1.310 mmol) in dioxane/H₂O (10 mL, ratio: 4:1) using potassium isopropenyltrifluoroborate (1d)(339 mg, 2.293 mmol), potassium carbonate (453 mg, 3.28 mmol), PdCl₂(dppf)-CH₂Cl₂ adduct (214 mg, 0.262 mmol) and heating for 1 h at 150° C. in a microwave. This gave after work up and purification using flash column chromatography [silica gel (24 g), eluting with DMA-80 in DCM from 0-80%] (S)-1-(sec-butyl)-6-(prop-1-en-2-yl)-N-(1-(3,4,5-trimethoxyphenyl)-1H-imidazol-4-yl)-1H-pyrazolo[3,4-d]pyrimidin-4-amine (22c) (360 mg, 59% yield) as a white solid; MS (ES+): 464.20 (M+1); (ES−): 462.20 (M−1).

### Step-3: Preparation of(S)-1-(sec-butyl)-6-isopropyl-N-(1-(3,4,5-trimethoxyphenyl)-1H-imidazol-4-yl)-1H-pyrazolo[3,4-d]pyrimidin-4-amine (22d)

Compound 22d was prepared according to the procedure reported in step-3 of scheme 1, from (S)-1-(sec-butyl)-6-(prop-1-en-2-yl)-N-(1-(3,4,5-trimethoxyphenyl)-1H-imidazol-4-yl)-1H-pyrazolo[3,4-d]pyrimidin-4-amine (22c) (0.35 g, 0.755 mmol) in MeOH (20 mL) using palladium hydroxide on carbon, 20 wt. % loading (dry basis), matrix carbon, wet support (0.106 g, 0.151 mmol) and stirring for 2 days at RT under a H₂ atmosphere. This gave after work up and purification using flash column chromatography [silica gel (12 g), eluting with DMA-80 in DCM from 0-50%] followed by purification using reverse phase column chromatography [C18 column (50 g), eluting with ACN in water (containing 0.1% HCl) from 0-100%] (S)-1-(sec-butyl)-6-isopropyl-N-(1-(3,4,5-trimethoxyphenyl)-1H-imidazol-4-yl)-1H-pyrazolo[3,4-d]pyrimidin-4-amine (22d)(130 mg, 37% yield) HCl salt as a white solid; ¹H NMR (300 MHz, DMSO-d₆) δ 11.79 (s, 1H), 8.59-8.43 (m, 2H), 8.09 (d, J=1.7 Hz, 1H), 7.00 (s, 2H), 4.85 (q, J=6.8 Hz, 1H), 3.88 (s, 6H), 3.70 (s, 3H), 3.17 (p, J=6.9 Hz, 1H), 2.00-1.77 (m, 2H), 1.46 (d, J=6.7 Hz, 3H), 1.38 (d, J=6.8 Hz, 6H), 0.68 (t, J=7.3 Hz, 3H); MS (ES+): 466.20 (M+1); MS (ES−): 464.20 (M−1); Chiral HPLC: AD-H column 70/30 [(0.1% DEA in Hexane in 0.1% DEA in ethanol)] 1.0 mL/min UV detection 256 nm, 30 mins run time (Temp 40° C.); compound 19d [Rt=13.80 (peak-1), 0.09%]; compound 22d [Rₜ=22.27 (peak-2); 99.91%]; 99.82% ee; Optical rotation: [α]D=(+) 21.88 [CH₃OH, 0.165]; Analysis calculated for C₂₄H₃₁N₇O₃. 1HCl·2H₂O. C, 53.58; H, 6.74; Cl, 6.59; N, 18.22; Found: C, 53.79; H, 6.48; Cl, 6.54; N, 18.15.

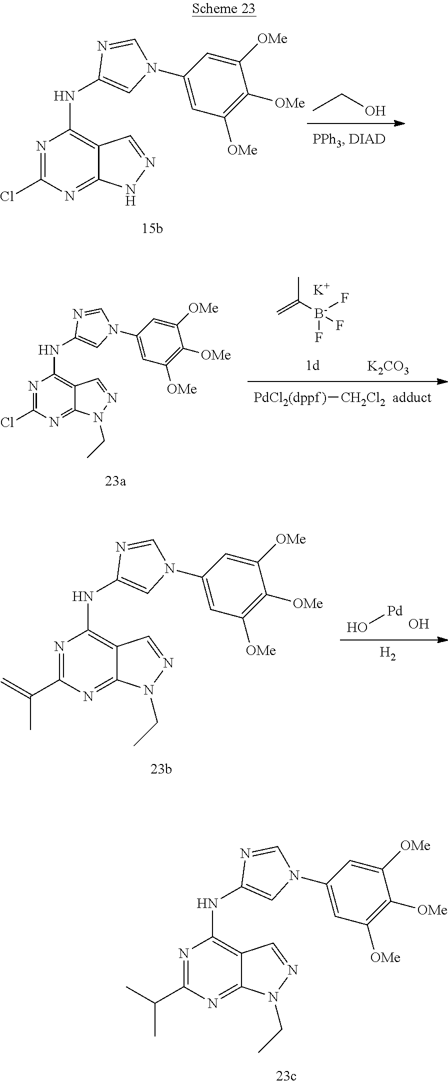

### Preparation of 1-ethyl-6-isopropyl-N-(1-(3,4,5-trimethoxyphenyl)-1H-imidazol-4-yl)-1H-pyrazolo[3,4-d]pyrimidin-4-amine (23c)

### Step-1: Preparation of 6-chloro-1-ethyl-N-(1-(3,4,5-trimethoxyphenyl)-1H-imidazol-4-yl)-1H-pyrazolo[3,4-d]pyrimidin-4-amine (23a)

Compound 23a was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (1 g, 2.489 mmol) in THF (20 mL) using triphenylphosphine, ethanol, DIAD. This gave after work up and purification using flash column chromatography [silica gel (80 g), eluting with DMA-80 in DCM from 0-60%] 6-chloro-1-ethyl-N-(1-(3,4,5-trimethoxyphenyl)-1H-imidazol-4-yl)-1H-pyrazolo[3,4-d]pyrimidin-4-amine (23a) (0.5 g, 47% yield) as a yellow solid; ¹H NMR (300 MHz, DMSO-d₆) δ 11.38 (s, 1H), 8.44 (s, 1H), 8.18 (s, 1H), 7.87 (d, J=1.6 Hz, 1H), 6.92 (s, 2H), 4.31 (q, J=7.2 Hz, 2H), 3.87 (s, 6H), 3.69 (s, 3H), 1.38 (t, J=7.2 Hz, 3H); MS (ES+): 430.10 (M+1); MS (ES−): 429.10 (M−1).

### Step-2: Preparation of 1-ethyl-6-(prop-1-en-2-yl)-N-(1-(3,4,5-trimethoxyphenyl)-1H-imidazol-4-yl)-1H-pyrazolo[3,4-d]pyrimidin-4-amine (23b)

Compound 23b was prepared according to the procedure reported in step-1 of scheme 1, from 6-chloro-1-ethyl-N-(1-(3,4,5-trimethoxyphenyl)-1H-imidazol-4-yl)-1H-pyrazolo[3,4-d]pyrimidin-4-amine (23a) (0.5 g, 1.163 mmol) in dioxane/H₂O (10 mL, ratio: 4:1) using potassium isopropenyltrifluoroborate (1d)(301 mg, 2.036 mmol), potassium carbonate (402 mg, 2.91 mmol), PdCl₂(dppf)-CH₂Cl₂ adduct (190 mg, 0.233 mmol) and heating for 1 h at 150° C. in a microwave. This gave after work up and purification by flash column chromatography [silica gel (24 g), eluting with DMA-80 in DCM from 0-80%] 1-ethyl-6-(prop-1-en-2-yl)-N-(1-(3,4,5-trimethoxyphenyl)-1H-imidazol-4-yl)-1H-pyrazolo[3,4-d]pyrimidin-4-amine (23b) (220 mg, 43% yield); MS (ES+): 436.15 (M+1).

### Step-3: Preparation of 1-ethyl-6-isopropyl-N-(1-(3,4,5-trimethoxyphenyl)-1H-imidazol-4-yl)-1H-pyrazolo[3,4-d]pyrimidin-4-amine (23c)

Compound 23c was prepared according to the procedure reported in step-3 of scheme 1, from 1-ethyl-6-(prop-1-en-2-yl)-N-(1-(3,4,5-trimethoxyphenyl)-1H-imidazol-4-yl)-1H-pyrazolo[3,4-d]pyrimidin-4-amine (23b) (0.22 g, 0.505 mmol) in MeOH (10 mL) using palladium hydroxide on carbon, 20 wt. % loading (dry basis), matrix carbon, wet support (0.071 g, 0.101 mmol) and stirring for 2 days at RT under a H₂ atmosphere. This gave after work up and purification using flash column chromatography [silica gel (12 g), eluting with DMA-80 in DCM from 0-50%] followed by purification using reverse phase column chromatography [C18 column (50 g), eluting with ACN in water (containing 0.1% HCl) from 0-100%](1-ethyl-6-isopropyl-N-(1-(3,4,5-trimethoxyphenyl)-1H-imidazol-4-yl)-1H-pyrazolo[3,4-d]pyrimidin-4-amine (23c) (100 mg, 45% yield) HCl salt as a white solid; ¹H NMR (300 MHz, DMSO-d₆) δ 12.06 (s, 1H, D₂O exchangeable), 8.60 (s, 1H), 8.51 (s, 1H), 8.09 (d, J=1.6 Hz, 1H), 7.01 (s, 2H), 4.40 (q. J=7.2 Hz, 2H), 3.88 (s, 6H), 3.70 (s, 3H), 3.19 (h, J=6.9 Hz, 1H), 1.44-1.36 (m, 9H); MS (ES+): 438.20 (M+1); Analysis calculated for C₂₂H₂₇N₇O₃. 1.1HCl·1.25H₂O. C, 52.84; H, 6.17; Cl, 7.80; N, 19.60; Found: C, 52.88; H, 6.17; Cl, 7.92; N, 19.49.

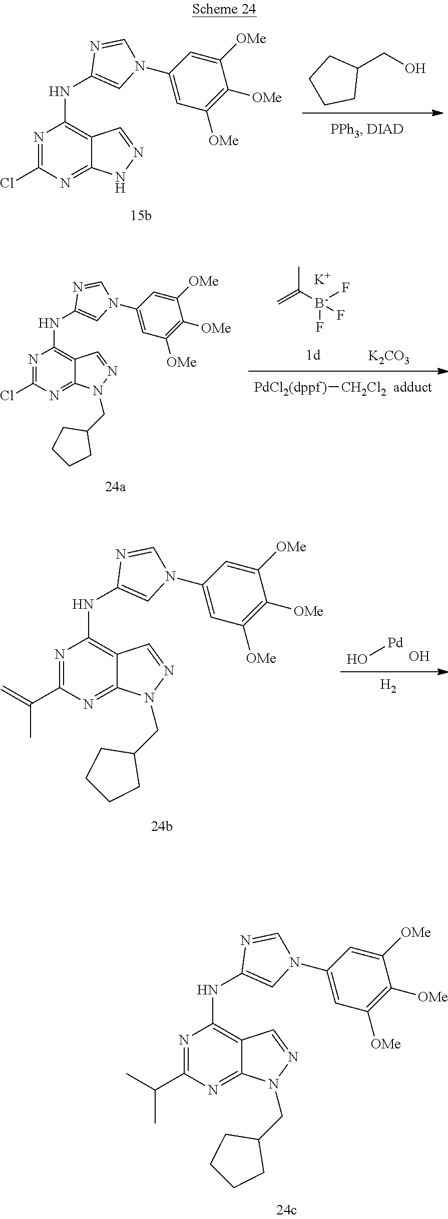

### Preparation of 1-(cyclopentylmethyl)-6-isopropyl-N-(1-(3,4,5-trimethoxyphenyl)-1H-imidazol-4-yl)-1H-pyrazolo[3,4-d]pyrimidin-4-amine (24c)

### Step-1: Preparation of 6-chloro-1-(cyclopentylmethyl)-N-(1-(3,4,5-trimethoxyphenyl)-1H-imidazol-4-yl)-1H-pyrazolo[3,4-d]pyrimidin-4-amine (24a)

Compound 24a was prepared according to the procedure reported in step-2 of scheme 15, from 6-chloro-N-(1-(3,4,5-trimethoxyphenyl)-1H-imidazol-4-yl)-1H-pyrazolo[3,4-d]pyrimidin-4-amine (15b) (1 g, 2.489 mmol) in THF (20 mL) using triphenylphosphine, cyclopentylmethanol, DIAD. This gave after work up and purification using flash column chromatography [silica gel (80 g), eluting with DMA-80 in DCM from 0-60%] 6-chloro-1-(cyclopentylmethyl)-N-(1-(3,4,5-trimethoxyphenyl)-1H-imidazol-4-yl)-1H-pyrazolo[3,4-d]pyrimidin-4-amine (24a) (0.52 g, 43% yield) as a yellow solid; MS (ES+): 484.20 (M+1).

### Step-2: Preparation of 1-(cyclopentylmethyl)-6-(prop-1-en-2-yl)-N-(1-(3,4,5-trimethoxyphenyl)-1H-imidazol-4-yl)-1H-pyrazolo[3,4-d]pyrimidin-4-amine (24b)

Compound 24b was prepared according to the procedure reported in step-1 of scheme 1, from 6-chloro-1-(cyclopentylmethyl)-N-(1-(3,4,5-trimethoxyphenyl)-1H-imidazol-4-yl)-1H-pyrazolo[3,4-d]pyrimidin-4-amine (24a) (0.52 g, 1.074 mmol) in dioxane/H₂O (10 mL, ratio: 4:1) using potassium isopropenyltrifluoroborate (1d) (278 mg, 1.880 mmol), potassium carbonate (371 mg, 2.69 mmol), PdCl₂(dppf)-CH₂Cl₂ adduct (175 mg, 0.215 mmol) and heating for 1 h at 150° C. in a microwave. This gave after work up and purification using flash column chromatography [silica gel (24 g), eluting with DMA-80 in DCM from 0-80%] 1-(cyclopentylmethyl)-6-(prop-1-en-2-yl)-N-(1-(3,4,5-trimethoxyphenyl)-1H-imidazol-4-yl)-1H-pyrazolo[3,4-d]pyrimidin-4-amine (24b) (220 mg, 42% yield) as a white solid; MS (ES+): 490.20 (M+1).

### Step-3: Preparation of 1-(cyclopentylmethyl)-6-isopropyl-N-(1-(3,4,5-trimethoxyphenyl)-1H-imidazol-4-yl)-1H-pyrazolo[3,4-d]pyrimidin-4-amine (24c)

Compound 24c was prepared according to the procedure reported in step-3 of scheme 1, from 1-(cyclopentylmethyl)-6-(prop-1-en-2-yl)-N-(1-(3,4,5-trimethoxyphenyl)-1H-imidazol-4-yl)-1H-pyrazolo[3,4-d]pyrimidin-4-amine (24b) (0.22 g, 0.449 mmol) in MeOH (10 mL) using palladium hydroxide on carbon, 20 wt. % loading (dry basis), matrix carbon, wet support (0.063 g, 0.090 mmol) and stirring for 2 days at RT under a H₂ atmosphere. This gave after work up and purification using flash column chromatography [silica gel (12 g), eluting with DMA-80 in DCM from 0-50%] followed by purification using reverse phase column chromatography [C18 column (50 g), eluting with ACN in water (containing 0.1% HCl) from 0-100%] 1-(cyclopentylmethyl)-6-isopropyl-N-(1-(3,4,5-trimethoxyphenyl)-1H-imidazol-4-yl)-1H-pyrazolo[3,4-d]pyrimidin-4-amine (24c) (120 mg, 54% yield) HCl salt as a white solid; ¹H NMR (300 MHz, DMSO-d₆) δ 12.11 (s, 1H), 8.62 (s, 1H), 8.53 (s, 1H), 8.08 (d, J=1.6 Hz, 1H), 7.02 (s, 2H), 4.29 (d, J=7.4 Hz, 2H), 3.88 (s, 6H), 3.70 (s, 3H), 3.21 (h, J=6.9 Hz, 1H), 2.47-2.39 (m, 1H), 1.67-1.44 (m, 6H), 1.37 (d, J=6.8 Hz, 6H), 1.34-1.19 (m, 2H); MS (ES+): 492.25 (M+1); Analysis calculated for C₂₆H₃₃N₇O₃·1HCl·1.5H₂O. C, 56.26; H, 6.72; Cl, 6.39; N, 17.66; Found: C, 56.09; H, 6.70; Cl, 6.21; N, 17.56.

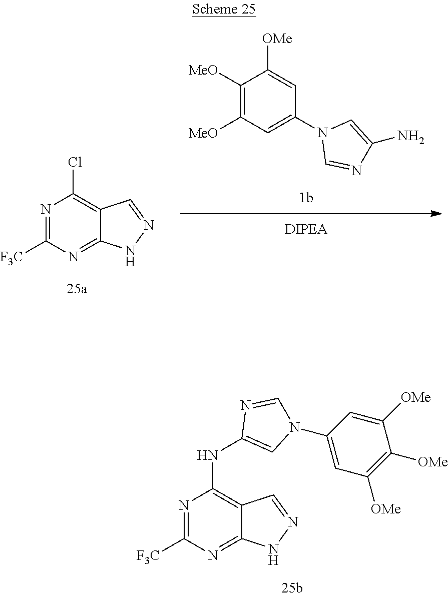 [SMILES: FC(F)(F)c1nc(Cl)c2cn[nH]c2n1 ; COc1cc(-n2cnc(N)c2)cc(OC)c1OC ; COc1cc(-n2cnc(Nc3nc(C(F)(F)F)nc4[nH]ncc34)c2)cc(OC)c1OC]

### Preparation of 6-(trifluoromethyl)-N-(1-(3,4,5-trimethoxyphenyl)-1H-imidazol-4-yl)-1H-pyrazolo[3,4-d]pyrimidin-4-amine (25b)

Compound 25b was prepared according to the procedure reported in step-1 of scheme 1, from 4-chloro-6-(trifluoromethyl)-1H-pyrazolo[3,4-d]pyrimidine (25a) (238 mg, 1.069 mmol; CAS #1780-80-9) in 2-propanol (8 mL) using DIPEA (0.560 mL, 3.21 mmol), 1-(3,4,5-trimethoxyphenyl)-1H-imidazol-4-amine (1b) (293 mg, 1.176 mmol) and heating at 95° C. for 3 h. This gave after work up and purification using reverse phase column chromatography [C18 column (50 g), eluting with ACN in water (containing 0.1% HCl) from 0-100%] 6-(trifluoromethyl)-N-(1-(3,4,5-trimethoxyphenyl)-1H-imidazol-4-yl)-1H-pyrazolo[3,4-d]pyrimidin-4-amine (25b) (311 mg, 67% yield) HCl salt as a white solid; ¹H NMR (300 MHz, DMSO-d₆) δ 11.62 (s, 1H), 8.61 (s, 1H), 8.45 (s, 1H), 8.05 (s, 1H), 6.96 (s, 2H), 3.88 (s, 6H), 3.71 (s, 3H); ¹⁹F NMR (282 MHz, DMSO) δ −69.07. MS (ES+): 436.1 (M+1); MS (ES−): 434.1 (M−1); Analysis calculated for C₁₈H₁₆F₃N₇O₃·HCl: C, 45.82; H, 3.63; Cl, 7.51; N, 20.78; Found: C, 45.63; H, 3.80; Cl, 7.21; N, 20.46.

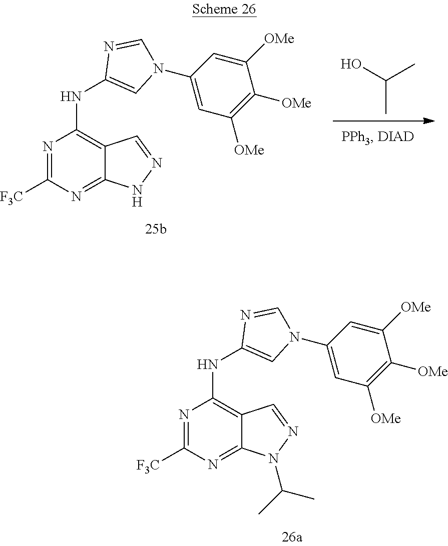

### Preparation of 1-isopropyl-6-(trifluoromethyl)-N-(1-(3,4,5-trimethoxyphenyl)-1H-imidazol-4-yl)-1H-pyrazolo[3,4-d]pyrimidin-4-amine

Compound 26a was prepared according to the procedure reported in step-2 of scheme 15, from 6-(trifluoromethyl)-N-(1-(3,4,5-trimethoxyphenyl)-1H-imidazol-4-v)-1H-pyrazolo[3,4-d]pyrimidin-4-amine (25b) (310 mg, 0.712 mmol) in THF (10 mL) using triphenylphosphine (224 mg, 0.854 mmol), propan-2-ol (51.3 mg, 0.854 mmol), DIAD (0.166 mL, 0.854 mmol). This gave after work up and purification using flash column chromatography [silica gel (12 g), eluting with MeOH in DCM from 0-15%] followed by purification using reverse phase column chromatography [C18 column (50 g), eluting with ACN in water (containing 0.1% HCl) from 0-100%] 1-isopropyl-6-(trifluoromethyl)-N-(1-(3,4,5-trimethoxyphenyl)-1H-imidazol-4-yl)-1H-pyrazolo[3,4-d]pyrimidin-4-amine (26a) (137 mg, 40% yield) HCl salt as a white solid; ¹H NMR (300 MHz, DMSO-d₆) δ 11.74 (s, 1H, D₂O exchangeable), 8.78-8.42 (m, 2H), 8.07 (s, 1H), 6.99 (s, 2H), 5.10 (m, J=6.7 Hz, 1H), 3.89 (s, 6H), 3.72 (s, 3H), 1.50 (d, J=6.6 Hz, 6H)¹⁹F NMR (282 MHz, DMSO-d₆) δ −68.91. MS (ES+): 478.2 (M+1); MS (ES−): 476.2 (M−1); Analysis calculated for C₂₁H₂₂F₃N₇O₃·0.75HCl: C, 49.97; H, 4.54; Cl, 5.27; N, 19.42; Found: C, 49.68; H, 4.80; Cl, 5.26; N, 19.24.

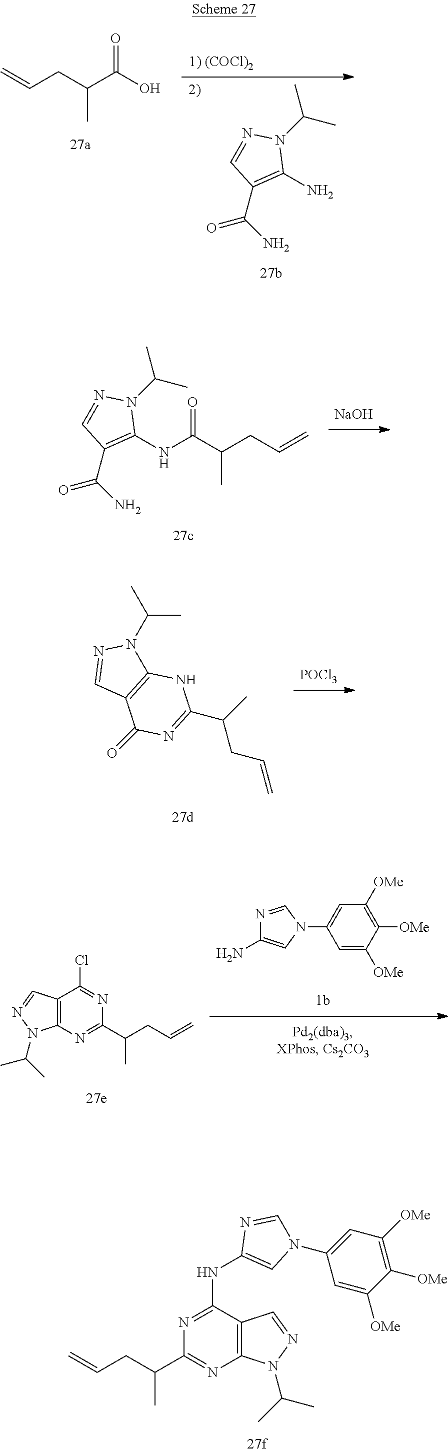

### Preparation of 1-isopropyl-6-(pent-4-en-2-yl)-N-(1-(3,4,5-trimethoxyphenyl)-1H-imidazol-4-yl)-1H-pyrazolo[3,4-d]pyrimidin-4-amine (27f)

### Step-1: Preparation of 1-isopropyl-5-(2-methylpent-4-enamido)-1H-pyrazole-4-carboxamide (27c)

To a stirred solution of 2-methylpent-4-enoic acid (27a) (5.0 g, 43.80 mmol; CAS #1575-74-2) in DCM (100 mL) at 0° C. was added drop-wise oxalyl chloride (16.68 g, 131.41 mmol), DMF (0.5 mL) and stirred at RT for 1.5 h. The reaction mixture was concentrated under nitrogen at RT and diluted with 1,4-dioxane (50 mL). This mixture was added drop wise to a stirred solution of 5-amino-1-isopropyl-1H-pyrazole-4-carboxamide (27b) (5.89 g, 35.04 mmol; CAS #21254-24-0) in 1,4 dioxane (50 mL) at RT and stirred for 12 h at RT. The reaction mixture was diluted with water (250 mL) extracted with ethyl acetate (250 mL×2). The combined organic layers were washed with IN HCl solution (250 mL), brine (50 mL), dried, filtered, concentrated in vacuum and the residue obtained was triturated with n-heptane. The solid obtained was collected by filtration, dried to give 1-isopropyl-5-(2-methylpent-4-enamido)-1H-pyrazole-4-carboxamide (27c) (2.11 g, 18.22%) as an off-white solid; ¹H NMR (300 MHz, DMSO-d₆) δ 9.75 (s, 1H), 7.87 (s, 1H), 7.23 (s, 1H), 6.98 (s, 1H), 5.92-5.72 (m, 1H), 5.02 (d, J=11.6 Hz, 2H), 4.29 (p, J=6.5 Hz, 1H), 2.73-2.60 (m, 1H), 2.47-2.31 (m, 1H), 2.21-2.06 (m, 1H), 1.30 (dd, J=6.5, 1.4 Hz, 6H), 1.11 (d, J=6.8 Hz, 3H).

### Step-2: Preparation of 1-isopropyl-6-(pent-4-en-2-yl)-1,7-dihydro-4H-pyrazolo[3,4-d]pyrimidin-4-one (27d)

A mixture of 1-isopropyl-5-(2-methylpent-4-enamido)-1H-pyrazole-4-carboxamide (27c) (0.53 g, 2.0 mmol) in NaOH (2N) (0.8 g, 20.0 mmol) was heated at 100° C. for 1 h. The reaction mixture was cooled to RT and IN HCl was added until the pH of the mixture was acidic. The aqueous layer was extracted with ethyl acetate (50 mL×2), washed with brine (50 mL), dried, filtered and concentrated to give 1-isopropyl-6-(pent-4-en-2-yl)-1,7-dihydro-4H-pyrazolo[3,4-d]pyrimidin-4-one (27d) (0.5 g, 99% yield) as a light brown solid; ¹H NMR (300 MHz, DMSO-d₆) δ 11.98 (s, 1H), 7.98 (s, 1H), 5.85-5.65 (m, 1H), 5.08-4.86 (m, 3H), 2.96-2.81 (m, 1H), 2.38-2.22 (m, 1H), 1.44 (dd, J=6.7, 4.4 Hz, 6H), 1.24 (d, J=6.7 Hz, 4H), 0.86 (t, J=5.7 Hz, OH).

### Step-3: Preparation of 4-chloro-1-isopropyl-6-(pent-4-en-2-yl)-1H-pyrazolo[3,4-d]pyrimidine (27e)

A mixture of 1-isopropyl-6-(pent-4-en-2-yl)-1,7-dihydro-4H-pyrazolo[3,4-d]pyrimidin-4-one (27d) (1.88 g, 7.63 mmol) and POCl₃ (45 mL, 482.75 mmol) was heated for 1 h at 100° C. The reaction mixture was cooled to RT poured into ice water and the pH was adjusted to basic using a solution of NaHCO₃. The reaction mixture was extracted with ethyl acetate (500 mL×2), washed with brine (500 mL), dried, filtered and concentrated in vacuum. The residue obtained was purified using flash column chromatography [silica gel, eluting with EtOAc in heptane from 0-5%] to give 4-chloro-1-isopropyl-6-(pent-4-en-2-yl)-1H-pyrazolo[3,4-d] pyrimidine (27e) (1.44 g, 71% yield) as an oily mass; ¹H NMR (300 MHz, DMSO-d₆) δ 8.37 (s, 1H), 5.86-5.66 (m, 1H), 5.22-5.05 (m, 1H), 5.09-4.94 (m, 1H), 4.99-4.89 (m, 1H), 3.23-3.10 (m, 1H), 2.68-2.53 (m, 1H), 2.45-2.30 (m, 1H), 1.50 (dd, J=6.7, 1.8 Hz, 6H), 1.30 (d, J=6.9 Hz, 3H).

### Step-4: Preparation of 1-isopropyl-6-(pent-4-en-2-yl)-N-(1-(3,4,5-trimethoxyphenyl)-1H-imidazol-4-yl)-1H-pyrazolo[3,4-d]pyrimidin-4-amine (27f)

To a stirred solution of 4-chloro-1-isopropyl-6-(pent-4-en-2-yl)-1H-pyrazolo[3,4-d]pyrimidine (27e) (1.44 g, 5.43 mmol) in 1,4-dioxane (44.0 mL) was added 1-(3,4,5-trimethoxyphenyl)-1H-imidazol-4-amine (1 b) (1.76 g, 7.07 mmol), Pd₂(dba)₃ (0.994 g, 1.086 mmol), X-phos (1.035 g, 2.17 mmol), Cs₂CO₃ (5.30 g, 16.29 mmol) and heated at 120° C. for 4 h under nitrogen. The reaction mixture was cooled to RT, filtered through a small pad of Celite and the filtrate was concentrated in vacuo. The obtained residue was purified using flash column chromatography [silica gel, eluting with 10% MeOH in DCM], crystallized using IPA and further purified using reverse phase column chromatography [C18 column (130 g), eluting with ACN in water (containing 0.1% HCl) from 0-100%] to give 1-isopropyl-6-(pent-4-en-2-yl)-N-(1-(3,4,5-trimethoxyphenyl)-1H-imidazol-4-yl)-1H-pyrazolo[3,4-d]pyrimidin-4-amine (27f) (0.170 g, 68% yield) HCl salt as a white solid; ¹H NMR (300 MHz, DMSO-d₆) δ 11.08 (s, 1H, D₂O exchangeable), 8.47-8.25 (m, 2H), 8.10 (d, J=1.5 Hz, 1H), 6.95 (s, 2H), 5.95-5.69 (m, 1H), 5.16-4.83 (m, 3H), 3.87 (s, 6H), 3.69 (s, 3H), 3.06 (q, J=6.9 Hz, 1H), 2.76-2.61 (m, 1H), 2.40 (m, 1H), 1.46 (d, J=6.6 Hz, 6H), 1.34 (d, J=6.9 Hz, 3H); MS (ES+): 478.15 (M+1); MS (ES−): 476.20 (M−1); Analysis calculated for C₂₃H₃₁N₇O₃·HCl·1.75H₂O. C, 55.04; H, 6.56; Cl, 6.50; N, 17.97; Found: C, 55.03; H, 6.35; Cl, 6.28; N, 17.63.

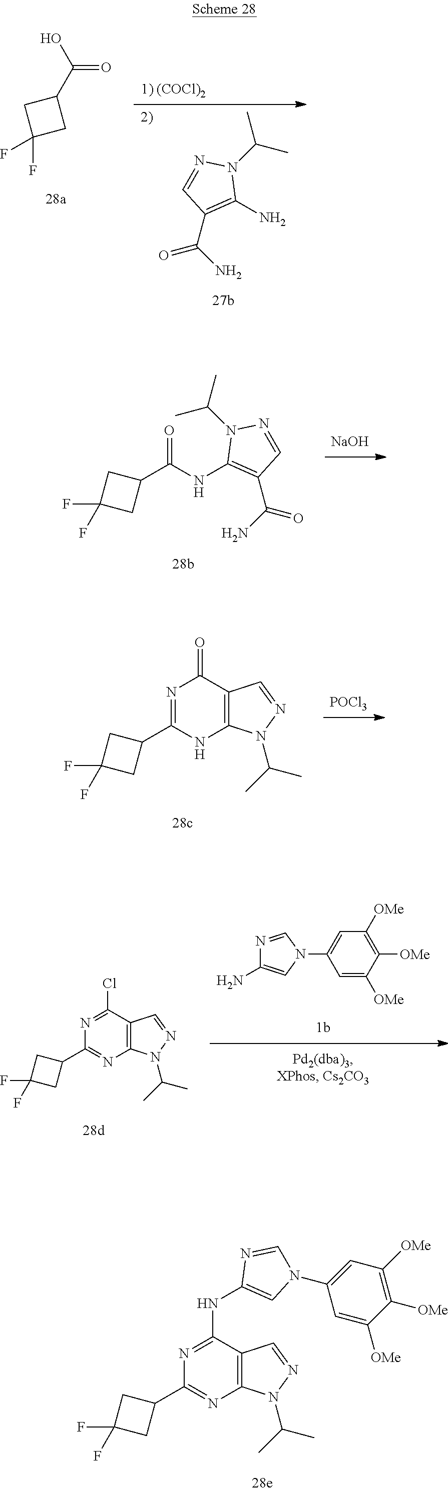

### Preparation of 6-(3,3-difluorocyclobutyl)-1-isopropyl-N-(1-(3,4,5-trimethoxyphenyl)-1H-imidazol-4-yl)-1H-pyrazolo[3,4-d]pyrimidin-4-amine (28e)

### Step-1: Preparation of 5-(3,3-difluorocyclobutanecarboxamido)-1-isopropyl-1H-pyrazole-4-carboxamide (28b)

Compound 28b was prepared according to the procedure reported in step-1 of scheme 27, from 3,3-difluorocyclobutanecarboxylic acid (28a) (1.0 g, 7.34 mmol) in DCM (20.0 mL) using oxalyl chloride (2.79 g, 22.04 mmol), 5-amino-1-isopropyl-1H-pyrazole-4-carboxamide (27b) (0.88 g, 5.24 mmol) in 1,4-dioxane (30 mL) and stirring at RT for 12 h. This gave after work up and purification using column chromatography [silica gel, eluting with 5% MeOH in DCM] 5-(3,3-difluorocyclobutanecarboxamido)-1-isopropyl-1H-pyrazole-4-carboxamide (28b) (500 mg, 33% yield) as an off-white solid; ¹H NMR (300 MHz, DMSO-d₆) δ 9.89 (s, 1H), 7.90 (s, 1H), 7.35 (s, 1H), 6.97 (s, 1H), 4.32 (p, J=6.6 Hz, 1H), 3.01-2.68 (m, 5H), 1.31 (d, J=6.6 Hz, 6H).

### Step-2: Preparation of 6-(3,3-difluorocyclobutyl)-1-isopropyl-1H-pyrazolo[3,4-d]pyrimidin-4 (7H)-one (28c)

Compound 28c was prepared according to the procedure reported in step-2 of scheme 27, from 5-(3,3-difluorocyclobutanecarboxamido)-1-isopropyl-1H-pyrazole-4-carboxamide (28b) (500 mg, 1.74 mmol) using an aqueous solution of NaOH (2N) (0.69 g, 17.46 mmol) and heating at 70° C. for 0.5 h. This gave after work up 6-(3,3-difluorocyclobutyl)-1-isopropyl-1H-pyrazolo[3,4-d]pyrimidin-4 (7H)-one (28c) (300 mg, 65% yield) as an off white solid; ¹H NMR (300 MHz, DMSO-d₆) δ 12.14 (s, 1H), 8.01 (s, 1H), 5.10-4.86 (m, 1H), 3.54-3.37 (m, 1H), 3.18-2.79 (m, 4H), 1.45 (d, J=6.7 Hz, 6H).

### Step-3: Preparation of 4-chloro-6-(3,3-difluorocyclobutyl)-1-isopropyl-1H-pyrazolo[3,4-d]pyrimidine (28d)

Compound 28d was prepared according to the procedure reported in step-3 of scheme 27, from 6-(3,3-difluorocyclobutyl)-1-isopropyl-1H-pyrazolo[3,4-d]pyrimidin-4 (7H)-one (28c) (0.5 g, 1.86 mmol) using POCl₃ (16.28 g, 106.23 mmol) and heating at 100° C. for 1 h. This gave after work up and purification using column chromatography [silica gel, eluting with 10% MeOH in DCM] 4-chloro-6-(3,3-difluorocyclobutyl)-1-isopropyl-1H-pyrazolo[3,4-d]pyrimidine (28d) (300 mg, 57% yield) as an oil; ¹H NMR (300 MHz, DMSO-d₆) δ 8.36 (s, 1H), 5.08 (hept, J=6.6 Hz, 1H), 3.63 (qd, J=8.6, 3.4 Hz, 1H), 3.08-2.83 (m, 4H), 1.44 (d, J=6.7 Hz, 6H).

### Step-4: Preparation of 6-(3,3-difluorocyclobutyl)-1-isopropyl-N-(1-(3,4,5-trimethoxyphenyl)-1H-imidazol-4-yl)-1H-pyrazolo[3,4-d]pyrimidin-4-amine (28e)

Compound 28e was prepared according to the procedure reported in step-4 of scheme 27, from 4-chloro-6-(3,3-difluorocyclobutyl)-1-isopropyl-1H-pyrazolo[3,4-d]pyrimidine (28d) (0.3 g, 1.04 mmol) in 1,4-dioxane (9.0 mL) using 1-(3,4,5-trimethoxyphenyl)-1H-imidazol-4-amine (1 b) (0.34 g, 1.36 mmol), Pd₂(dba)₃ (0.191 g, 0.20 mmol), X-phos (0.197 g 0.418 mmol), Cs₂CO₃ (1.02 g, 3.13 mmol) and heating at 120° C. for 4 h under nitrogen. This gave after work up, purification using column chromatography [silica gel, eluting with 10% MeOH in DCM], crystallization using IPA and final purification using reverse phase column chromatography [C18 column (130 g), eluting with ACN in water (containing 0.1% HCl) from 0-100%] 6-(3,3-difluorocyclobutyl)-1-isopropyl-N-(1-(3,4,5-trimethoxyphenyl)-1H-imidazol-4-yl)-1H-pyrazolo[3,4-d]pyrimidin-4-amine (28e) (0.184 g, 94% yield) HCl salt as a white solid; ¹H NMR (300 MHz, DMSO-d₆) δ 11.01 (s, 1H, D₂O exchangeable), 8.43 (s, 1H), 8.28 (s, 1H), 8.06 (s, 1H), 6.94 (s, 2H), 5.14-4.95 (m, 1H), 3.87 (s, 6H), 3.69 (s, 3H), 3.61-3.45 (m, 1H), 3.20-2.96 (m, 4H), 1.45 (d, J=6.7 Hz, 6H); ¹⁹F NMR (282 MHz, DMSO-d₆) δ −78.64, −93.38; MS (ES+): 500.10 (M+1); MS (ES−): 498.10 (M−1); Analysis calculated for C₂₄H₂₇F₂N₇O₃·0.95HCl·1.25H₂O: C, 51.78; H, 5.51; Cl, 6.05; N, 17.61; Found: C, 51.60; H, 5.34; Cl, 5.86; N, 17.51.

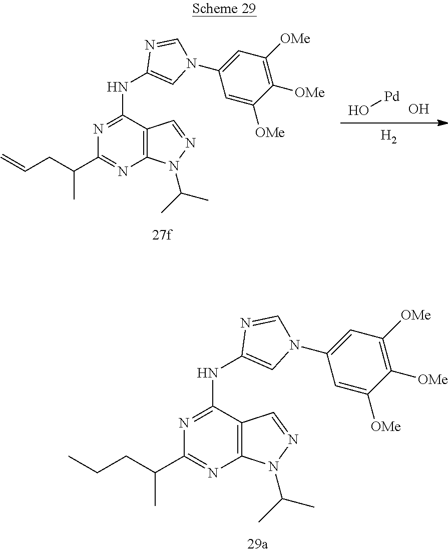

### Preparation of 1-isopropyl-6-(pentan-2-yl)-N-(1-(3,4,5-trimethoxyphenyl)-1H-imidazol-4-yl)-1H-pyrazolo[3,4-d]pyrimidin-4-amine (29a)

Compound 29a was prepared according to the procedure reported in step-3 of scheme 1, from 1-isopropyl-6-(pent-4-en-2-yl)-N-(1-(3,4,5-trimethoxyphenyl)-1H-imidazol-4-yl)-1H-pyrazolo[3,4-d]pyrimidin-4-amine (27f) (0.5 g, 1.046 mmol) in MeOH (15 mL) using Pd(OH)₂ (20% in H₂O) (0.294 g, 0.209 mmol) and stirring for 3 days at RT under a H₂ atmosphere. This gave after work up and purification using flash column chromatography [silica gel (12 g), eluting with EtOAc in n-heptane from 50-100%], crystallizing using IPA and final purification using reverse phase column chromatography [C18 column (130 g), eluting with ACN in water (containing 0.1% HCl) from 0-100%] 1-isopropyl-6-(pentan-2-yl)-N-(1-(3,4,5-trimethoxyphenyl)-1H-imidazol-4-yl)-1H-pyrazolo[3,4-d]pyrimidin-4-amine (29a) (0.225 g, 74% yield) HCl salt as a white solid; ¹H NMR (300 MHz, DMSO-d₆) δ 11.14 (s, 1H, D₂O exchangeable), 8.44-8.26 (m, 2H), 8.09 (s, 1H), 6.94 (s, 2H), 5.09-4.98 (m, 11H), 3.87 (s, 6H), 3.70 (s, 3H), 3.05-2.88 (m, 1H), 1.95-1.77 (m, 1H), 1.72-1.53 (m, 1H), 1.46 (d, J=6.7 Hz, 6H), 1.33 (d, J=6.9 Hz, 3H), 1.31-1.19 (m, 2H), 0.86 (t, J=7.3 Hz, 3H); MS (ES+): 480.20 (M+1); MS (ES−): 478.15 (M−1); Analysis calculated for C₂₅H₃₃N₇O₃·HCl·1.25H₂O. C, 55.75; H, 6.83; Cl, 6.58; N, 18.21; Found: C, 55.97; H, 6.62; Cl, 6.38; N, 18.10.

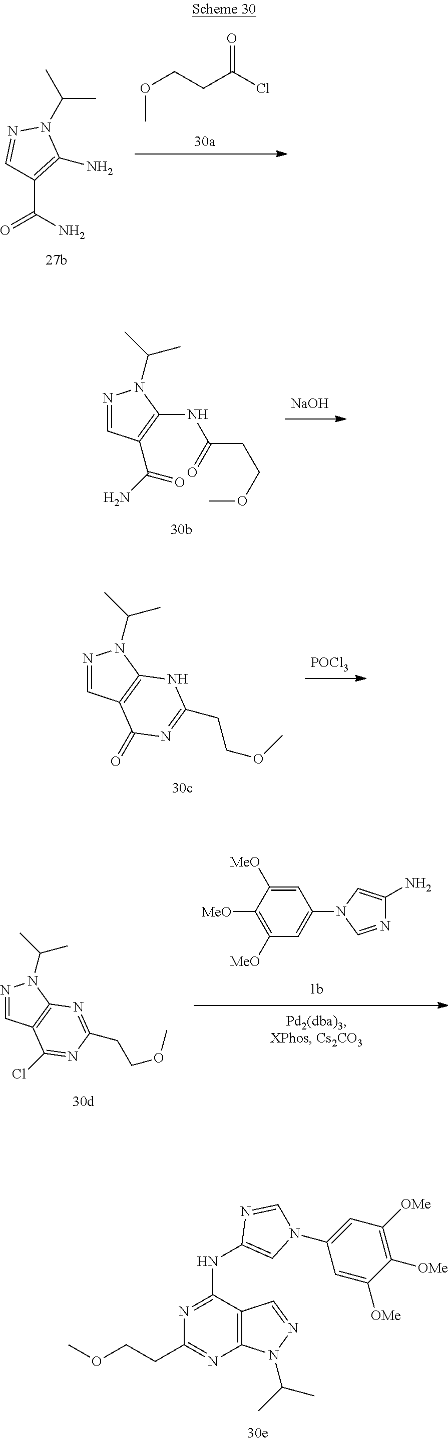

### Preparation of 1-isopropyl-6-(2-methoxyethyl)-N-(1-(3,4,5-trimethoxyphenyl)-1H-imidazol-4-yl)-1H-pyrazolo[3,4-d]pyrimidin-4-amine (30e)

### Step-1: Preparation of 1-isopropyl-5-(3-methoxypropanamido)-1H-pyrazole-4-carboxamide (30b)

To a solution of 5-amino-1-isopropyl-1H-pyrazole-4-carboxamide (27b) (1.0 g, 5.94 mmol) in 1,4 dioxane (30 mL) was added a solution of 3-methoxypropanoyl chloride (30a) (1.02, 8.32 mmol, CAS #4244-59-1) drop wise in 1,4 dioxane and stirred for 12 h at RT. The reaction mixture was concentrated in vacuum and the residue obtained was purified using column chromatography [silica gel, eluting with 10% MeOH in DCM] to give 1-isopropyl-5-(3-methoxypropanamido)-1H-pyrazole-4-carboxamide (30b)(700 mg, 46% yield) as an off-white solid. ¹H NMR (300 MHz, DMSO-d₆) δ 9.86 (s, 1H), 7.85 (s, 1H), 7.09 (d, J=13.8 Hz, 2H), 4.62-4.22 (m, 1H), 3.62 (t, J=6.2 Hz, 2H), 3.26 (s, 3H), 2.59 (t, J=6.2 Hz, 2H), 1.31 (d, J=6.6 Hz, 6H).

### Step-2: Preparation of 1-isopropyl-6-(2-methoxyethyl)-1H-pyrazolo[3,4-d]pyrimidin-4 (7H)-one (30c)

Compound 30c was prepared according to the procedure reported in step-2 of scheme 27, from 1-isopropyl-5-(3-methoxypropanamido)-1H-pyrazole-4-carboxamide (30b)(700 mg, 2.75 mmol) using an aqueous solution of NaOH (2N) (1.10 g, 27.52 mmol) and heating at 70° C. for 0.5 h. This gave after work up 1-isopropyl-6-(2-methoxyethyl)-1H-pyrazolo[3,4-d]pyrimidin-4 (7H)-one (30c) (550 mg, 85.40% yield) as an off white solid, 1H NMR (300 MHz, DMSO-d₆) δ 12.15 (s, 1H), 8.02 (s, 1H), 5.08-4.78 (m, 1H), 4.04 (t, J=6.6 Hz, 2H), 3.35 (s, 3H), 3.14 (t, J=6.6 Hz, 2H), 1.44 (d, J=6.7 Hz, 6H).

### Step-3: Preparation of 4-chloro-1-isopropyl-6-(2-methoxyethyl)-1H-pyrazolo[3,4-d]pyrimidine (30d)

Compound 30d was prepared according to the procedure reported in step-3 of scheme 27, from 1-isopropyl-6-(2-methoxyethyl)-1H-pyrazolo[3,4-d]pyrimidin-4 (7H)-one (30c) (1.0 g, 4.32 mmol) using POCl₃ (36.98 g, 241.23 mmol) and heating at 90° C. for 1 h. This gave after work up and purification using column chromatography [silica gel, eluting with 10% MeOH in DCM] 4-chloro-1-isopropyl-6-(2-methoxyethyl)-1H-pyrazolo[3,4-d]pyrimidine (30d) (350 mg, 41%) as an oil; ¹H NMR (300 MHz, DMSO-d₆) δ 8.43 (s, 1H), 5.35-4.88 (m, 1H), 4.15 (t, J=6.5 Hz, 2H), 3.45 (t, J=6.5 Hz, 2H), 3.33 (s, 3H), 1.51 (d, J=6.7 Hz, 6H).

### Step-4: Preparation of 1-isopropyl-6-(2-methoxyethyl)-N-(1-(3,4,5-trimethoxyphenyl)-1H-imidazol-4-yl)-1H-pyrazolo[3,4-d]pyrimidin-4-amine (30e)

Compound 30e was prepared according to the procedure reported in step-4 of scheme 27, from 4-chloro-1-isopropyl-6-(2-methoxyethyl)-1H-pyrazolo[3,4-d]pyrimidine (30d) (0.35 g, 1.37 mmol) in 1,4-dioxane (10.5 mL) using 1-(3,4,5-trimethoxyphenyl)-1H-imidazol-4-amine (1b) (0.45 g, 1.78 mmol), Pd₂(dba)₃ (0.25 g, 0.27 mmol), X-phos (0.26 g, 0.54 mmol), Cs₂CO₃ (1.34 g, 4.12 mmol) and heating at 90° C. for 4 h. This gave after work up and purification using column chromatography [silica gel, eluting with 10% MeOH in DCM], crystallizing using IPA and final purification using reverse phase column chromatography [C18 column (30 g), eluting with ACN in water (containing 0.1% HCl) from 0-100%] 1-isopropyl-6-(2-methoxyethyl)-N-(1-(3,4,5-trimethoxyphenyl)-1H-imidazol-4-yl)-1H-pyrazolo[3,4-d]pyrimidin-4-amine (30e) (0.121 g, 78.1% yield) HCl salt as a white solid; ¹H NMR (300 MHz, DMSO-d₆) δ 11.18 (s, 1H, D₂O exchangeable), 8.48-8.29 (m, 2H), 8.06 (s, 1H), 6.98 (s, 2H), 5.10-4.97 (m, 1H), 3.93-3.89 (m, 2H), 3.88 (s, 6H), 3.70 (s, 3H), 3.26 (s, 3H), 3.10 (t, J=6.5 Hz, 2H), 1.45 (d, J=6.7 Hz, 6H); MS (ES+): 468.10 (M+1); MS (ES−): 466.10 (M−1); Analysis calculated for C₂₃H₂₉N₇O₄·HCl·2H₂O: C, 51.16; H, 6.35; Cl, 6.56; N, 18.16; Found: C, 51.06; H, 6.23. Cl, 6.35; N, 17.97.

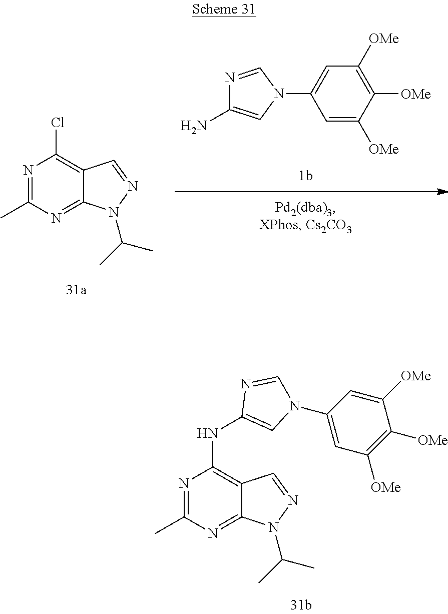 [SMILES: Cc1nc(Cl)c2cnn(C(C)C)c2n1 ; COc1cc(-n2cnc(N)c2)cc(OC)c1OC ; COc1cc(-n2cnc(Nc3nc(C)nc4c3cnn4C(C)C)c2)cc(OC)c1OC]

### Preparation of 1-isopropyl-6-methyl-N-(1-(3,4,5-trimethoxyphenyl)-1H-imidazol-4-yl)-1H-pyrazolo[3,4-d]pyrimidin-4-amine (31b)

Compound 31b was prepared according to the procedure reported in step-4 of scheme 27, from 4-chloro-1-isopropyl-6-methyl-1H-pyrazolo[3,4-d]pyrimidine (31a) (160 mg, 0.760 mmol; CAS #1251212-42-6) in toluene (8 mL) and t-Butanol (2 mL) using 1-(3,4,5-trimethoxyphenyl)-1H-imidazol-4-amine (1b) (379 mg, 1.519 mmol), Pd₂(dba)₃ (139 mg, 0.152 mmol), X-phos (145 mg, 0.304 mmol), Cs₂CO₃ (619 mg, 1.899 mmol) and heating at 90° C. for 4 h. This gave after work up and purification using flash column chromatography [silica gel (12 g), eluting with MeOH in DCM from 0-15%] followed by purification using reverse phase column chromatography [C18 column (50 g), eluting with ACN in water (containing 0.1% HCl) from 0-100%]l-isopropyl-6-methyl-N-(1-(3,4,5-trimethoxyphenyl)-1H-imidazol-4-yl)-1H-pyrazolo[3,4-d]pyrimidin-4-amine (31b) (0.240 g, 75% yield) HCl salt as a white yellow solid; ¹H NMR (300 MHz, DMSO-d₆) δ 12.50 (s, 1H, D₂O exchangeable), 9.00-8.44 (m, 2H), 8.05 (d, J=1.7 Hz, 1H), 7.06 (s, 2H), 5.19-5.06 (m, 1H), 3.90 (s, 6H), 3.71 (s, 3H), 2.68 (s, 3H), 1.47 ((d, J=6.6 Hz, 6H); MS (ES+): 424.2 (M+1); MS (ES−): 422.2 (M−1). Analysis calculated for C₂₁H₂₅N₇O₃·HCl·1.6H₂O: C, 51.61; H, 6.02; Cl, 7.25; N, 20.06; Found: C, 51.58; H, 5.94; Cl, 7.15; N, 19.84.

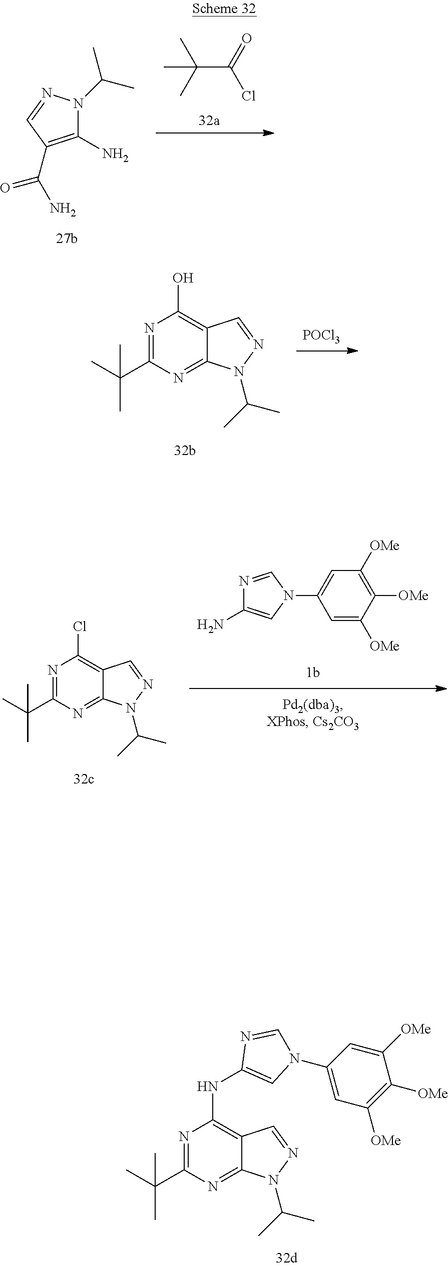

### Preparation of 6-(tert-butyl)-1-isopropyl-N-(1-(3,4,5-trimethoxyphenyl)-1H-imidazol-4-yl)-1H-pyrazolo[3,4-d]pyrimidin-4-amine (32d)

### Step-1: Preparation of 6-(tert-butyl)-1-isopropyl-1H-pyrazolo[3,4-d]pyrimidin-4-ol (32b)

Compound 32b was prepared according to the procedure reported in step-1 of scheme 30, from 5-amino-1-isopropyl-1H-pyrazole-4-carboxamide (27b) (376 mg, 2.235 mmol) in 1,4 dioxane (10 mL) using pivaloyl chloride (32a) (411 mg, 3.41 mmol), stirring at RT for 2 h followed by heating to 120° C. in a sealed tube for 24 h. This gave after work up and purification using flash column chromatography [silica gel (12 g), eluting with EtOAc/MeOH (9:1) in hexanes from 20-100%] 6-(tert-butyl)-1-isopropyl-1H-pyrazolo[3,4-d]pyrimidin-4-ol (32b) (162 mg, 20% yield) as a yellow solid; MS (ES+): 235.10 (M+1); MS (ES−): 233.10 (M−1).

### Step-2: Preparation of 6-(tert-butyl)-4-chloro-1-isopropyl-1H-pyrazolo[3,4-d]pyrimidine (32c)

Compound 32c was prepared according to the procedure reported in step-3 of scheme 27, from 6-(tert-butyl)-1-isopropyl-1H-pyrazolo[3,4-d]pyrimidin-4-ol (32b) (162 mg, 0.691 mmol) using POCl₃ (5 mL, 53.6 mmol) and heating at 100° C. for 1 h. This gave after work up and purification using flash column chromatography [silica gel (12 g), eluting with EtOAc in hexane from 0-40%] 6-(tert-butyl)-4-chloro-1-isopropyl-1H-pyrazolo[3,4-d]pyrimidine (32c) (74 mg, 42% yield) as a colorless oil; ¹H NMR (300 MHz, DMSO-d₆) δ 8.36 (s, 1H), 5.19-4.99 (m, 1H), 1.52 (d, J=6.7 Hz, 6H), 1.41 (s, 9H); MS (ES+): 253.10 (M+1).

### Step-3: Preparation of 6-(tert-butyl)-1-isopropyl-N-(1-(3,4,5-trimethoxyphenyl)-1H-imidazol-4-yl)-1H-pyrazolo[3,4-d]pyrimidin-4-amine (32d)

Compound 32d was prepared according to the procedure reported in step-4 of scheme 27, from 6-(tert-butyl)-4-chloro-1-isopropyl-1H-pyrazolo[3,4-d]pyrimidine (32c) (70 mg, 0.277 mmol) in toluene (8 mL) and t-Butanol (2 mL) using 1-(3,4,5-trimethoxyphenyl)-1H-imidazol-4-amine (1b) (138 mg, 0.554 mmol), Pd₂(dba)₃ (50.7 mg, 0.055 mmol), X-phos (52.8 mg, 0.111 mmol), Cs₂CO3 (226 mg, 0.692 mmol) and heating at 110° C. for 4 h. This gave after work up and purification using flash column chromatography [silica gel (12 g), eluting with EtOAc/MeOH (9:1) in hexanes from 0-100%] followed by purification using reverse phase column chromatography [C18 column (50 g), eluting with ACN in water (containing 0.1% HCl) from 0-100%] 6-(tert-butyl)-1-isopropyl-N-(1-(3,4,5-trimethoxyphenyl)-1H-imidazol-4-yl)-1H-pyrazolo[3,4-d]pyrimidin-4-amine (32d) (58 mg, 45% yield) HCl salt as a white yellow solid; ¹H NMR (300 MHz, DMSO-d₆) δ 11.89 (s, 1H, D₂O exchangeable), 8.70-8.37 (m, 2H), 8.08 (s, 1H), 7.00 (s, 2H), 5.19-4.97 (m, 1H), 3.88 (s, 6H), 3.71 (s, 3H), 1.56-1.26 (m, 15H); MS (ES+): 466.2 (M+1).

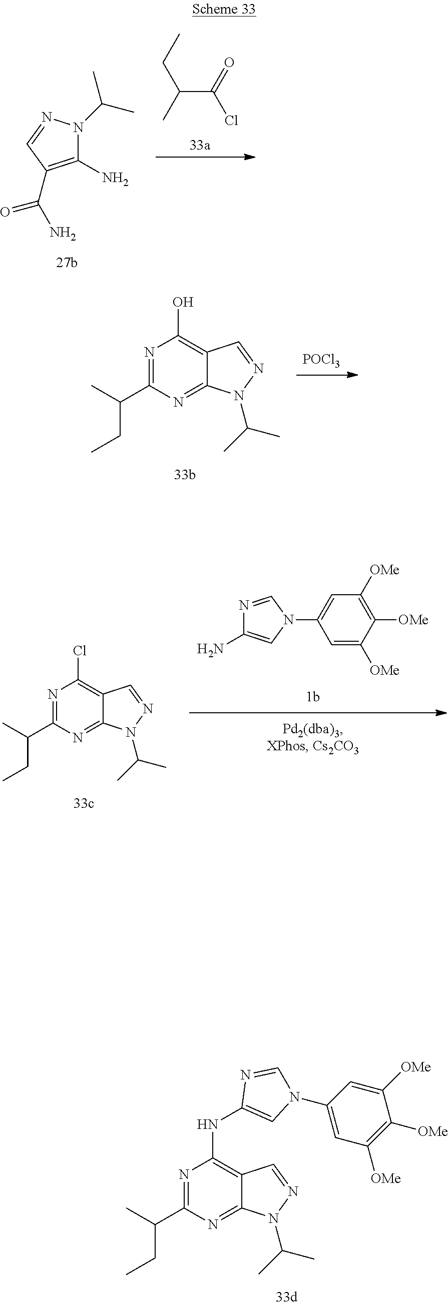

### Preparation of 6-(sec-butyl)-1-isopropyl-N-(1-(3,4,5-trimethoxyphenyl)-1H-imidazol-4-yl)-1H-pyrazolo[3,4-d]pyrimidin-4-amine (33d)

### Step-1: Preparation of 6-(sec-butyl)-1-isopropyl-1H-pyrazolo[3,4-d]pyrimidin-4-ol (33b)

Compound 33b was prepared according to the procedure reported in step-1 of scheme 30, from 5-amino-1-isopropyl-1H-pyrazole-4-carboxamide (27b) (316 mg, 1.879 mmol) in 1,4 dioxane (8 mL) using 2-methylbutanoyl chloride (33a) (411 mg, 3.41 mmol; CAS #57526-28-0), stirring at RT for 2 h and heating to 120° C. in a sealed tube for 24 h. This gave after work up and purification using flash column chromatography [silica gel (12 g), eluting with EtOAc/MeOH (9:1) in hexanes from 0-100%] 6-(sec-butyl)-1-isopropyl-1H-pyrazolo[3,4-d]pyrimidin-4-ol (33b) (274 mg, 62% yield) as a yellow solid; ¹H NMR (300 MHz, DMSO-d₆) δ 11.94 (s, 1H), 7.98 (s, 1H), 5.04-4.77 (m, 1H), 2.81-2.64 (m, 1H), 1.83-1.69 (m, 1H), 1.65-1.50 (m, 1H), 1.44 (dd, J=6.7, 1.7 Hz, 6H), 1.23 (d, J=6.9 Hz, 3H), 0.84 (t, J=7.4 Hz, 3H); MS (ES+): 235.10 (M+1); MS (ES−): 233.10 (M−1).

### Step-2: Preparation of 6-(sec-butyl)-4-chloro-1-isopropyl-1H-pyrazolo[3,4-d]pyrimidine (33c)

Compound 33c was prepared according to the procedure reported in step-3 of scheme 27, from 6-(sec-butyl)-1-isopropyl-1H-pyrazolo[3,4-d]pyrimidin-4-ol (33b) (274 mg, 1.169 mmol) using POCl₃ (6 mL, 64.4 mmol) and heating at 100° C. for 1 h. This gave after work up and purification using flash column chromatography [silica gel (12 g), eluting with EtOAc in hexane from 0-40%] 6-(sec-butyl)-4-chloro-1-isopropyl-1H-pyrazolo[3,4-d]pyrimidine (33c) (226 mg 0.76% yield) as a colorless oil; ¹H NMR (300 MHz, DMSO-d₄) δ 8.37 (s, 1H), 5.28-4.84 (m, 1H), 3.08-2.89 (m, 1H), 1.94-1.75 (m, 1H), 1.75-1.56 (m, 1H), 1.51 (d, 61H), 1.30 (d, J=6.9 Hz, 3H), 0.83 (t, J=7.4 Hz, 3H); MS (ES+): 253.10 (M+1).

### Step-3: Preparation of 6-(sec-butyl)-1-isopropyl-N-(1-(3,4,5-trimethoxyphenyl)-1H-imidazol-4-yl)-1H-pyrazolo[3,4-d]pyrimidin-4-amine (33d)

Compound 33d was prepared according to the procedure reported in step-4 of scheme 27, from 6-(sec-butyl)-4-chloro-1-isopropyl-1H-pyrazolo[3,4-d]pyrimidine (33c)(220 mg, 0.870 mmol) in toluene (8 mL) and t-Butanol (2 mL) using 1-(3,4,5-trimethoxyphenyl)-1H-imidazol-4-amine (1b) (434 mg, 1.741 mmol), Pd₂(dba)₃ (159 mg, 0.174 mmol), XPhos (166 mg, 0.348 mmol), Cs₂CO3 (709 mg, 2.176 mmol) and heating at 110° C. for 4 h. This gave after work up and purification using flash column chromatography [silica gel (12 g), eluting with EtOAc/MeOH (9:1) in hexanes from 20-100%] followed by purification using reverse phase column chromatography [C18 column (50 g), eluting with ACN in water (containing 0.1% HCl) from 0-100%] 6-(sec-butyl)-1-isopropyl-N-(1-(3,4,5-trimethoxyphenyl)-1H-imidazol-4-yl)-1H-pyrazolo[3,4-d]pyrimidin-4-amine (33d) (195 mg, 48% yield) HCl salt as a white solid; ¹H NMR (300 MHz, DMSO-d₆) δ 12.11 (s, 1H, D₂O exchangeable), 8.81-8.41 (m, 2H), 8.06 (d, J=1.6 Hz, 1H), 7.02 (s, 2H), 5.18-5.07 (m, 1H), 3.89 (s, 6H), 3.71 (s, 3H), 3.08-2.91 (m, 1H), 2.01-1.81 (m, 1H), 1.81-1.60 (m, 1H), 1.48 (d, J=6.6 Hz, 6H), 1.37 (d, J=6.8 Hz, 3H), 0.91 (t, J=7.4 Hz, 3H); MS (ES+): 466.2 (M+1); Analysis calculated for: C₂₄H₃N₇O₃·HCl·H₂O: C, 55.43; H, 6.59; N, 18.85; Cl, 6.82; Found: C, 55.39; H, 6.61; N, 18.71; Cl, 6.69.

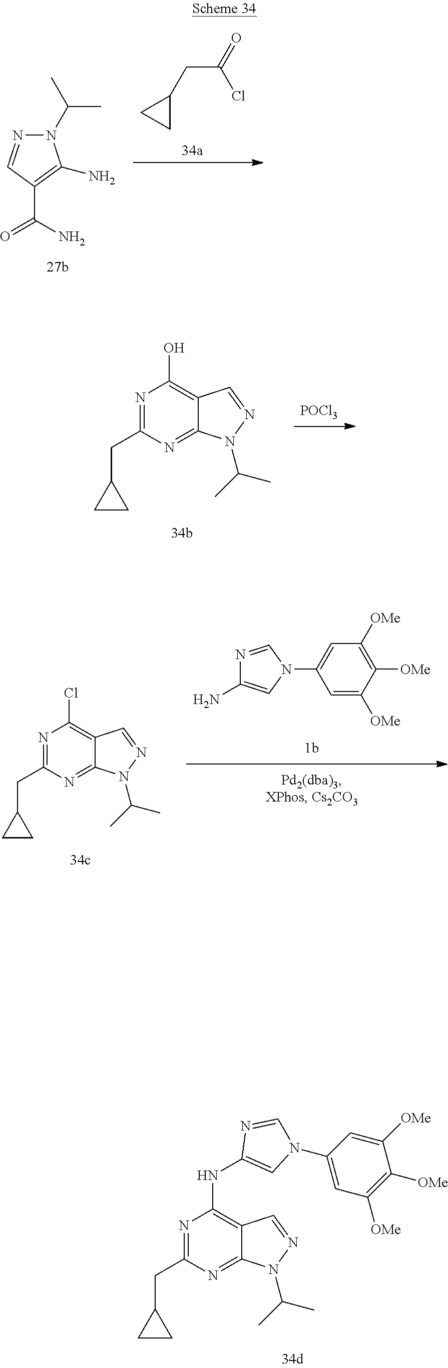

### Preparation of 6-(cyclopropylmethyl)-1-isopropyl-N-(1-(3,4,5-trimethoxyphenyl)-1H-imidazol-4-yl)-1H-pyrazolo[3,4-d]pyrimidin-4-amine (34d)

### Step-1: Preparation of 6-(cyclopropylmethyl)-1-isopropyl-1H-pyrazolo[3,4-d]pyrimidin-4-ol (34b)

Compound 34b was prepared according to the procedure reported in step-1 of scheme 30, from 5-amino-1-isopropyl-1H-pyrazole-4-carboxamide (27b) (305 mg, 1.813 mmol) in 1,4 dioxane (8 mL) using 2-cyclopropylacetyl chloride (34a) (298 mg, 2.51 mmol; CAS #54322-65-5), stirring at RT for 2 h and heating to 120° C. in a sealed tube for 24 h. This gave after work up and purification using flash column chromatography [silica gel (12 g), eluting with EtOAc/MeOH (9:1) in hexanes from 0-100%] 6-(cyclopropylmethyl)-1-isopropyl-1H-pyrazolo[3,4-d]pyrimidin-4-ol (34b) (277 mg, 66% yield) as a yellow solid; ¹H NMR (300 MHz, DMSO-d₆) δ 11.99 (s, 1H), 8.00 (s, 1H), 4.94 (p, J=6.7 Hz, 1H), 2.54-2.51 (m, 2H), 1.44 (d, J=6.7 Hz, 6H), 1.22-1.10 (m, 1H), 0.56-0.41 (m, 2H), 0.35-0.22 (m, 2H); MS (ES+): 233.10 (M+1); MS (ES−): 231.10 (M−1).

### Step-2: Preparation of 4-chloro-6-(cyclopropylmethyl)-1-isopropyl-1H-pyrazolo[3,4-d]pyrimidine (34c)

Compound 34c was prepared according to the procedure reported in step-3 of scheme 27, from 6-(cyclopropylmethyl)-1-isopropyl-1H-pyrazolo[3,4-d]pyrimidin-4-ol (34b) (260 mg, 1.119 mmol) using POCl₃ (6 mL, 64.4 mmol) and heating at 100° C. for 1 h. This gave after work up and purification using flash column chromatography [silica gel (12 g), eluting with EtOAc in hexane from 0-30%] 4-chloro-6-(cyclopropylmethyl)-1-isopropyl-1H-pyrazolo[3,4-d]pyrimidine (34c) (199 mg, 71% yield) as a colorless oil; ¹H NMR (300 MHz, DMSO-d₆) δ 8.39 (s, 1H), 5.13 (p, J=6.6 Hz, 1H), 2.85 (d, J=7.0 Hz, 2H), 1.51 (d, J=6.7 Hz, 6H), 1.35-1.13 (m, 1H), 0.59-0.40 (m, 2H), 0.39-0.15 (m, 2H); MS (ES+): 251.10 (M+1).

### Step-3: Preparation of 6-(cyclopropylmethyl)-1-isopropyl-N-(1-(3,4,5-trimethoxyphenyl)-1H-imidazol-4-yl)-1H-pyrazolo[3,4-d]pyrimidin-4-amine (34d)

Compound 34d was prepared according to the procedure reported in step-4 of scheme 27, from 4-chloro-6-(cyclopropylmethyl)-1-isopropyl-1H-pyrazolo[3,4-d]pyrimidine (34c) (190 mg, 0.758 mmol) in toluene (8 mL) and t-Butanol (2 mL) using 1-(3,4,5-trimethoxyphenyl)-1H-imidazol-4-amine (1b) (378 mg, 1.516 mmol), Pd₂(dba)₃ (139 mg, 0.152 mmol), XPhos (145 mg, 0.303 mmol), Cs₂CO3 (617 mg, 1.894 mmol) and heating at 110° C. for 4 h. This gave after work up and purification using flash column chromatography [silica gel (12 g), eluting with EtOAc/MeOH (9:1) in hexanes from 0-100%] followed by purification using reverse phase column chromatography [C18 column (50 g), eluting with ACN in water (containing 0.1% HCl) from 0-100%] 6-(cyclopropylmethyl)-1-isopropyl-N-(1-(3,4,5-trimethoxyphenyl)-1H-imidazol-4-yl)-1H-pyrazolo[3,4-d]pyrimidin-4-amine (34d) (149 mg, 42% yield) HCl salt as a white solid; ¹H NMR (300 MHz, DMSO-d₆) δ 12.46 (s, 1H, D₂O exchangeable), 8.59 (s, 2H), 8.05 (s, 1H), 7.02 (s, 2H), 5.20-5.01 (m, 1H), 3.89 (s, 6H), 3.70 (s, 3H), 2.88 (d, J=7.0 Hz, 2H), 1.48 (d, J=6.6 Hz, 6H), 1.36-1.17 (m, 1H), 0.71-0.53 (m, 2H), 0.53-0.31 (m, 2H); MS (ES+): 464.2 (M+1).

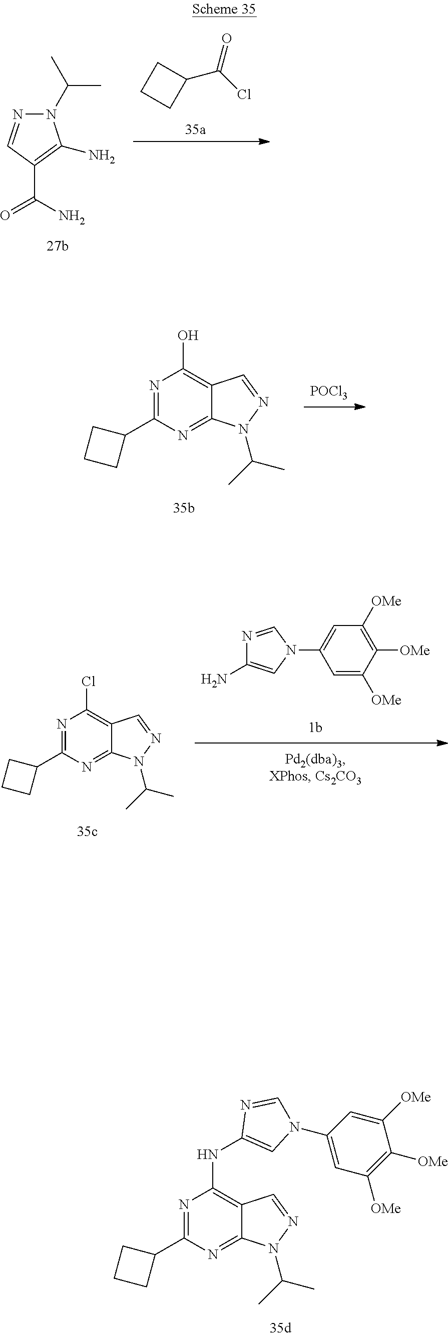

### Preparation of 6-cyclobutyl-1-isopropyl-N-(1-(3,4,5-trimethoxyphenyl)-1H-imidazol-4-yl)-1H-pyrazolo[3,4-d]pyrimidin-4-amine (35d)

### Step-1: Preparation of 6-cyclobutyl-1-isopropyl-1H-pyrazolo[3,4-d]pyrimidin-4-ol (35b)

Compound 35b was prepared according to the procedure reported in step-1 of scheme 30, from 5-amino-1-isopropyl-1H-pyrazole-4-carboxamide (27b) (300 mg, 1.784 mmol) in 1,4 dioxane (8 mL) using cyclobutanecarbonyl chloride (35a) (423 mg, 3.57 mmol; CAS #5006-22-4), stirring at RT for 2 h and heating to 120° C. in a sealed tube for 24 h. This gave after work up and purification using flash column chromatography [silica gel (12 g), eluting with EtOAc/MeOH (9:1) in hexanes from 0-100%] 6-cyclobutyl-1-isopropyl-1H-pyrazolo[3,4-d]pyrimidin-4-ol (35b) (343 mg, 83% yield) as a gray solid; ¹H NMR (300 MHz, DMSO-d₆) δ 11.89 (s, 1H), 7.98 (s, 1H), 5.07-4.88 (m, 1H), 3.60-3.44 (m, 1H), 2.47-2.31 (m, 2H), 2.31-2.17 (m, 2H), 2.06-1.92 (m, 1H), 1.92-1.74 (m, 1H), 1.46 (d, J=6.7 Hz, 6H); MS (ES+): 233.10 (M+1); MS (ES−): 231.10 (M−1).

### Step-2: Preparation of 4-chloro-6-cyclobutyl-1-isopropyl-1H-pyrazolo[3,4-d]pyrimidine (35c)

Compound 35c was prepared according to the procedure reported in step-3 of scheme 27, from 6-cyclobutyl-1-isopropyl-1H-pyrazolo[3,4-d]pyrimidin-4-ol (35b) (340 mg, 1.464 mmol) using POCl₃ (6 mL, 64.4 mmol) and heating at 100° C. for 1 h. This gave after work up and purification using flash column chromatography [silica gel (12 g), eluting with EtOAc in hexane from 0-30%] 4-chloro-6-cyclobutyl-1-isopropyl-1H-pyrazolo[3,4-d]pyrimidine (35c) (286 mg, 78% yield) as a colorless oil; ¹H NMR (300 MHz, DMSO-d₆) δ 8.38 (s, 1H), 5.26-4.97 (m, 1H), 3.97-3.69 (m, 1H), 2.48-2.28 (m, 4H), 2.14-1.99 (m, 1H), 1.99-1.81 (m, 1H), 1.51 (d, J=6.7 Hz, 6H); MS (ES+): 251.05 (M+1).

### Step-3: Preparation of 6-cyclobutyl-1-isopropyl-N-(1-(3,4,5-trimethoxyphenyl)-1H-imidazol-4-yl)-1H-pyrazolo[3,4-d]pyrimidin-4-amine (35d)

Compound 35d was prepared according to the procedure reported in step-4 of scheme 27, from 4-chloro-6-cyclobutyl-1-isopropyl-1H-pyrazolo[3,4-d]pyrimidine (35c) (280 mg, 1.117 mmol) in toluene (8 mL) and t-Butanol (2 mL) using 1-(3,4,5-trimethoxyphenyl)-1H-imidazol-4-amine (1b) (418 mg, 1.675 mmol), Pd₂(dba)₃ (205 mg, 0.223 mmol), XPhos (213 mg, 0.447 mmol), Cs₂CO₃ (910 mg, 2.79 mmol) and heating at 110° C. for 4 h. This gave after work up and purification using flash column chromatography [silica gel (12 g), eluting with EtOAc/MeOH (9:1) in hexanes from 0-100%] followed by purification using reverse phase column chromatography [C18 column (50 g), eluting with ACN in water (containing 0.1% HCl) from 0-100%] 6-cyclobutyl-1-isopropyl-N-(1-(3,4,5-trimethoxyphenyl)-1H-imidazol-4-yl)-1H-pyrazolo[3,4-d]pyrimidin-4-amine (35d) (208 mg, 40% yield) HCl salt as a white solid; ¹H NMR (300 MHz, DMSO-d₆) δ 12.58 (s, 1H, D₂O exchangeable), 8.83-8.42 (m, 2H), 8.08 (s, 1H), 7.04 (s, 2H), 5.17-5.09 (m, 1H), 3.88 (s, 7H), 3.70 (s, 3H), 2.55-2.35 (m, 4H), 2.20-2.01 (m, 1H), 2.01-1.82 (m, 1H), 1.47 (d, J=6.6 Hz, 6H); MS (ES+): 464.2 (M+1); Analysis calculated for: C₂₄H₂N₇O₃·1.05HCl·1.4H₂O: C, 54.69; H, 6.28; N, 18.60; Cl, 7.06; Found: C, 54.69; H, 6.31; N, 18.76; Cl, 6.96.

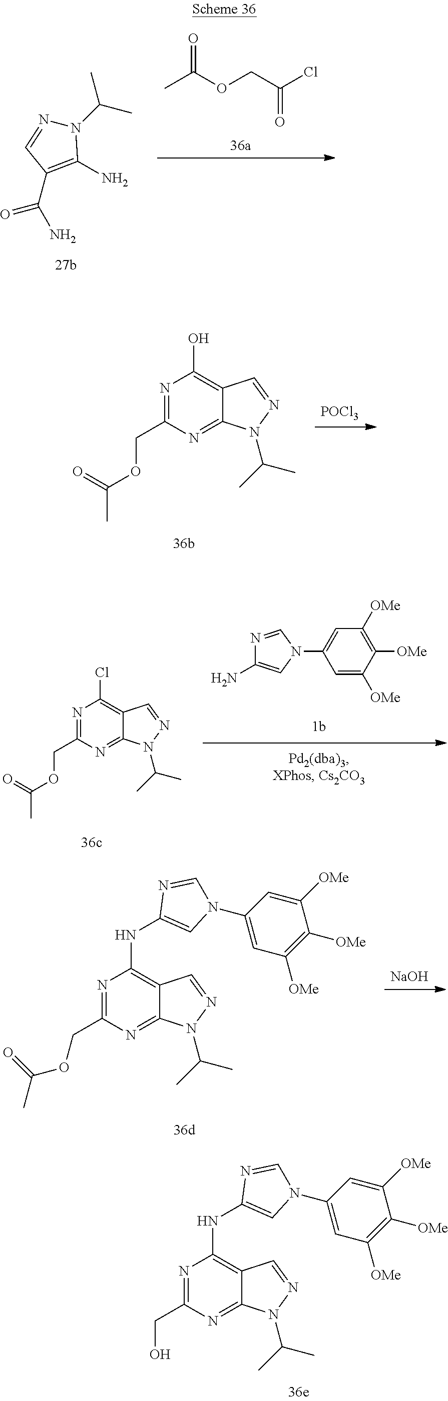

### Preparation of (1-isopropyl-4-((1-(3,4,5-trimethoxyphenyl)-1H-imidazol-4-yl)amino)-1H-pyrazolo[3,4-d]pyrimidin-6-yl)methanol (36e)

### Step-1: Preparation of (4-hydroxy-1-isopropyl-1H-pyrazolo[3,4-d]pyrimidin-6-yl)methyl acetate (36b)

Compound 36b was prepared according to the procedure reported in step-1 of scheme 30, from 5-amino-1-isopropyl-1H-pyrazole-4-carboxamide (27b) (316 mg, 1.879 mmol) in 1,4 dioxane (8 mL) using 2-chloro-2-oxoethyl acetate (36a) (1029 mg, 7.54 mmol; CAS #13831-31-7) stirring at RT for 20 min and heating to 120° C. in a scaled tube for 24 h. This gave after work up and purification using flash column chromatography [silica gel (12 g), eluting with EtOAc/MeOH (9:1) in hexanes from 0-100%] (4-hydroxy-1-isopropyl-1H-pyrazolo[3,4-d]pyrimidin-6-yl)methyl acetate (36b) (528 mg, 56% yield) as a colorless oil; MS (ES+): 251.05 (M+1); MS (ES−): 248.50 (M−1).

### Step-2: Preparation of (4-chloro-1-isopropyl-1H-pyrazolo[3,4-d]pyrimidin-6-yl)methyl acetate (36c)

Compound 36c was prepared according to the procedure reported in step-3 of scheme 27, from (4-hydroxy-1-isopropyl-1H-pyrazolo[3,4-d]pyrimidin-6-yl)methyl acetate (36b) (528 mg, 2.110 mmol) using POCl₃ (6 mL, 64.4 mmol) and heating at 100° C. for 1 h. This gave after work up and purification using flash column chromatography [silica gel (12 g), eluting with EtOAc in hexane from 0-40%] (4-chloro-1-isopropyl-1H-pyrazolo[3,4-d]pyrimidin-6-yl)methyl acetate (36c) (257 mg, 45% yield) as a colorless oil; ¹H NMR (300 MHz, DMSO-d₆) δ 8.47 (s, 1H), 5.32 (s, 2H), 5.19-5.04 (in, 1H), 2.19 (s, 3H), 1.51 (d, J=6.7 Hz, 6H); MS (ES+): 269.10 (M+1).

### Step-3: Preparation of (1-isopropyl-4-((1-(3,4,5-trimethoxyphenyl)-1H-imidazol-4-yl)amino)-1H-pyrazolo[3,4-d]pyrimidin-6-yl)methyl acetate (36d)

Compound 36d was prepared according to the procedure reported in step-4 of scheme 27, from (4-chloro-1-isopropyl-1H-pyrazolo[3,4-d]pyrimidin-6-yl)methyl acetate (36c)(254 mg, 0.945 mmol) in toluene (8 mL) and t-Butanol (2 mL) using 1-(3,4,5-trimethoxyphenyl)-1H-imidazol-4-amine (1b) (353 mg, 1.418 mmol), Pd₂(dba)₃ (173 mg, 0.189 mmol), X-phos (180 mg, 0.378 mmol) Cs₂CO₃ (770 mg, 2.363 mmol) and heating at 110° C. for 4 h. This gave after work up and purification using flash column chromatography [silica gel (12 g), eluting with EtOAc/MeOH (9:1) in hexanes from 0-100%] (1-isopropyl-4-((1-(3,4,5-trimethoxyphenyl)-1H-imidazol-4-yl)amino)-1H-pyrazolo[3,4-d]pyrimidin-6-yl)methyl acetate (36d) (455 mg, 100% yield) as a yellow oil; MS (ES+): 482.15 (M+1).

### Step-4: Preparation of (1-isopropyl-4-((1-(3,4,5-trimethoxyphenyl)-1H-imidazol-4-yl)amino)-1H-pyrazolo[3,4-d]pyrimidin-6-yl)methanol (36e)

Compound 36e was prepared according to the procedure reported in step-2 of scheme 27, from (1-isopropyl-4-((1-(3,4,5-trimethoxyphenyl)-1H-imidazol-4-yl)amino)-1H-pyrazolo[3,4-d]pyrimidin-6-yl)methyl acetate (36d) (455 mg, 0.945 mmol) in MeOH/THF (6 mL, 1:1) using a solution of NaOH (151 mg, 3.78 mmol) in water (2 mL) and stirring at RT for 3 h. This gave after work up and purification using flash column chromatography [silica gel (12 g), eluting with MeOH in DCM from 0-10%] followed by reverse phase column chromatography [C18 column (50 g), eluting with ACN in water (containing 0.1% HCl) from 0-100%] (1-isopropyl-4-((1-(3,4,5-trimethoxyphenyl)-1H-imidazol-4-yl)amino)-1H-pyrazolo[3,4-d]pyrimidin-6-yl)methanol (36e) (40 mg, 10% yield) HCl salt as a white yellow solid; ¹H NMR (300 MHz, DMSO-d₆) δ 13.44 (s, 1H, D₂O exchangeable), 8.77 (s, 1H), 8.62 (s, 1H), 7.95 (s, 1H), 7.04 (s, 2H), 5.07 (t, J=6.7 Hz, 1H), 4.74 (s, 2H), 3.89 (s, 6H), 3.70 (s, 3H), 1.48 (d, J=6.6 Hz, 6H); ¹H NMR (300 MHz, DMSO-d₆/D₂O) δ 8.73 (s, 1H), 8.60 (s, 11H), 7.97 (s, 1H), 7.04 (s, 2H), 5.06 (dd, J=13.3, 6.8 Hz, 1H), 4.73 (s, 2H), 3.89 (s, 6H), 3.70 (s, 3H), 1.48 (d, J=6.7 Hz, 6H). MS (ES+): 440.2 (M+1); Analysis calculated for C₂₁H₂₅N₇O₄·HCl·1.25H₂O: C, 50.60; H, 5.76; N, 19.67; Cl, 7.11; Found: C, 50.75; H, 5.62; N, 19.34; Cl, 6.99.

### Scheme 37

### Intentionally Left Blank.

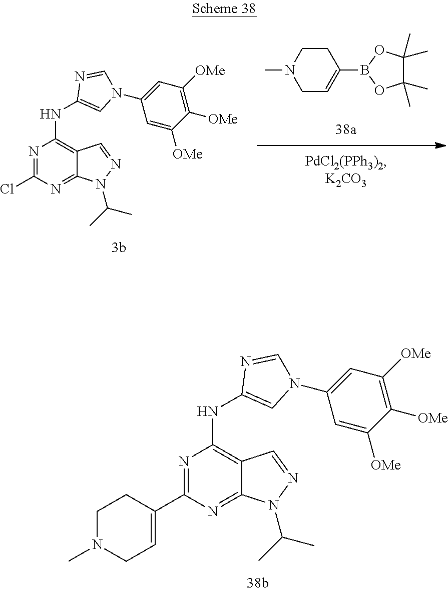

### Preparation of 1-isopropyl-6-(1-methyl-1,2,3,6-tetrahydropyridin-4-yl)-N-(1-(3,4,5-trimethoxyphenyl)-1H-imidazol-4-yl)-1H-pyrazolo[3,4-d]pyrimidin-4-amine (38b)

Compound 38b was prepared according to the procedure reported in step-2 of scheme 3, from 6-chloro-1-isopropyl-N-(1-(3,4,5-trimethoxyphenyl)-1H-imidazol-4-yl)-1H-pyrazolo[3,4-d]pyrimidin-4-amine (3b) (0.2 g, 0.45 mmol) in 1,4-dioxane (10 mL) using 1-methyl-4-(4,4,5,5-tetramethyl-1,3,2-dioxaborolan-2-yl)-1,2,3,6-tetrahydropyridine (38a) (125 mg, 0.56 mmol), a solution of potassium carbonate (186 mg, 1.35 mmol) in water (2 mL), bis(triphenylphosphine)palladium(II) chloride (63 mg, 0.90 mmol) and heating at 100° C. for 6 h under argon. This gave after work up and purification using column chromatography [silica gel, eluting with 10% MeOH in DCM] followed by purification using reverse phase column chromatography [C18 (130 g), eluting with ACN in water (containing 0.1% HCl) from 0-100%] to provide 1-isopropyl-6-(1-methyl-1,2,3,6-tetrahydropyridin-4-yl)-N-(1-(3,4,5-trimethoxyphenyl)-1H-imidazol-4-yl)-1H-pyrazolo[3,4-d]pyrimidin-4-amine (38b) (38 mg, 54% yield) HCl salt as a white solid; ¹H NMR (300 MHz, DMSO-d₆) δ 10.99 (s, 1H, D₂O exchangeable), 10.14 (s, 1H, D₂O exchangeable), 8.47 (s, 1H), 8.28 (d, J=1.4 Hz, 1H), 8.00 (s, 1H), 7.21 (s, 1H), 6.99 (s, 2H), 5.16-4.97 (m, 1H), 4.19-4.03 (m, 2H), 3.90 (s, 6H), 3.70 (s, 3H), 3.32-3.24 (m, 2H), 3.24-2.95 (m, 2H), 2.91 (d, J=4.7 Hz, 3H), 1.49 (d, J=6.7 Hz, 6H); MS (ES+): 505.20 (M+1); (ES−) 503.10 (M−1); Analysis calculated for C₂₆H₃₂N₈O₃·2.25HCl·3H₂O: C, 48.74; H, 6.33; N, 17.49; found: C, 48.51; H, 5.93; N, 17.37

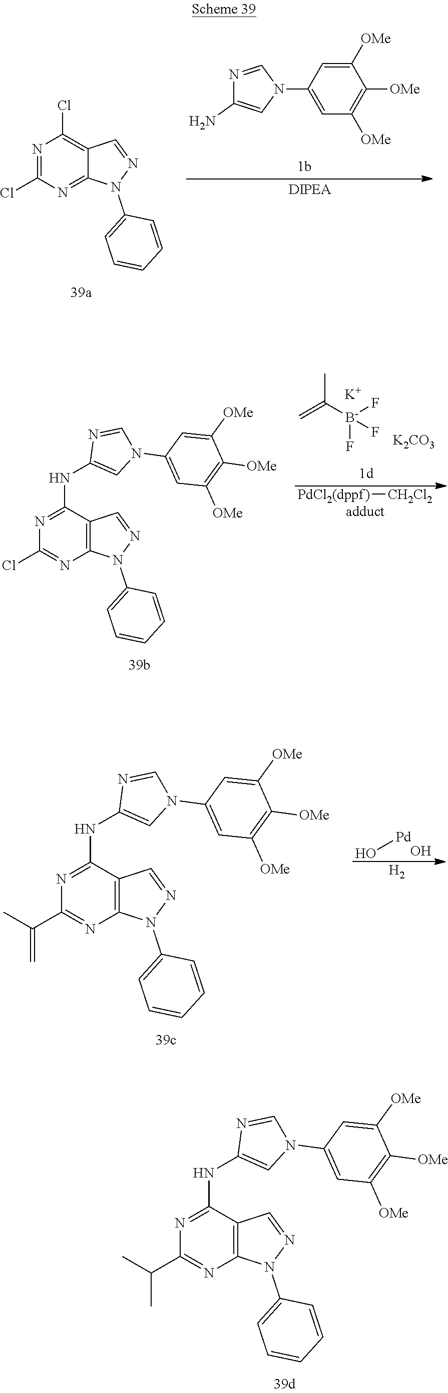

### Preparation of 6-isopropyl-1-phenyl-N-(1-(3,4,5-trimethoxyphenyl)-1H-imidazol-4-yl)-1H-pyrazolo[3,4-d]pyrimidin-4-amine (39d)

### Step-1: Preparation of 6-chloro-1-phenyl-N-(1-(3,4,5-trimethoxyphenyl)-1H-imidazol-4-yl)-1H-pyrazolo[3,4-d]pyrimidin-4-amine (39b)

Compound 39b was prepared according to the procedure reported in step-1 of scheme 1, from 4,6-dichloro-1-phenyl-1H-pyrazolo[3,4-d]pyrimidine (39a) (850 mg, 3.21 mmol; CAS #99971-84-3) in 2-propanol (20 mL) using DIPEA (1.680 mL, 9.62 mmol), 1-(3,4,5-trimethoxyphenyl)-1H-imidazol-4-amine (1b) (839 mg, 3.37 mmol) and refluxing for 12 h. This gave after work up 6-chloro-1-phenyl-N-(1-(3,4,5-trimethoxyphenyl)-1H-imidazol-4-yl)-1H-pyrazolo[3,4-d]pyrimidin-4-amine (39b) (1.1 g, 72% yield) as a yellow solid; MS (ES+): 478.10 (M+1); MS (ES−): 476.10 (M−1).

### Step-2: Preparation of 1-phenyl-6-(prop-1-en-2-yl)-N-(1-(3,4,5-trimethoxyphenyl)-1H-imidazol-4-yl)-1H-pyrazolo[3,4-d]pyrimidin-4-amine (39c)

Compound 39c was prepared according to the procedure reported in step-2 of scheme 1, from 6-chloro-1-phenyl-N-(1-(3,4,5-trimethoxyphenyl)-1H-imidazol-4-yl)-1H-pyrazolo[3,4-d]pyrimidin-4-amine (39b) (1 g, 2.092 mmol) in dioxane/H₂O (10 mL, ratio: 8:1) using potassium isopropenyltrifluoroborate (I d) (0.542 g, 3.66 mmol), potassium carbonate (0.723 g, 5.23 mmol), PdCl₂(dppf)-CH₂Cl₂ adduct (0.342 g, 0.418 mmol) and heating at 150° C. for 1.5 h in a microwave. This gave after work up and purification using flash column chromatography [silica gel (24 g), eluting with DMA-80 in DCM from 0-80%] 1-phenyl-6-(prop-1-en-2-yl)-N-(1-(3,4,5-trimethoxyphenyl)-1H-imidazol-4-yl)-1H-pyrazolo[3,4-d]pyrimidin-4-amine (39c) (180 mg, 18% yield); ¹H NMR (300 MHz, DMSO-d₆) δ 10.84 (s, 1H), 8.40 (s, 1H), 8.24 (d, J=1.6 Hz, 1H), 8.10 (d, J=1.6 Hz, 1H), 6.94 (s, 2H), 6.54-6.38 (m, 1H), 5.56 (dd, J=2.8, 1.6 Hz, 1H), 3.88 (s, 6H), 3.70 (s, 3H), 2.29 (s, 3H), 1.75 (s, 9H).

### Step-3: Preparation of 6-isopropyl-1-phenyl-N-(1-(3,4,5-trimethoxyphenyl)-1H-imidazol-4-yl)-1H-pyrazolo[3,4-d]pyrimidin-4-amine (39d)

Compound 39d was prepared according to the procedure reported in step-3 of scheme 1, from 1-phenyl-6-(prop-1-en-2-yl)-N-(1-(3,4,5-trimethoxyphenyl)-1H-imidazol-4-yl)-1H-pyrazolo[3,4-d]pyrimidin-4-amine (39c) (170 mg, 0.352 mmol) in MeOH (11 mL) using palladium hydroxide on carbon, 20 wt. % loading (dry basis), matrix carbon, wet support (54.3 mg, 0.077 mmol) and stirring overnight at RT under a H₂ atmosphere. This gave after work up and purification using flash column chromatography [silica gel (40 g), eluting with MeOH in DCM from 0-15%] followed by purification using reverse phase column chromatography [C18 column, eluting with ACN in water (containing 0.1% HCl) from 0-100%] 6-isopropyl-1-phenyl-N-(1-(3,4,5-trimethoxyphenyl)-1H-imidazol-4-yl)-1H-pyrazolo[3,4-d]pyrimidin-4-amine (39d) (125 mg, 73% yield) HCl salt as a white solid; ¹H NMR (300 MHz, DMSO-d₆) δ 11.17 (s, 1H, D₂O exchangeable), 8.67 (s, 1H), 8.36 (s, 1H), 8.31-8.22 (m, 2H), 8.18 (d, J=1.7 Hz, 1H), 7.64-7.47 (m, 2H), 7.40-7.24 (m, 1H), 6.97 (s, 2H), 3.88 (s, 6H), 3.70 (s, 3H), 3.25-3.09 (m, 1H), 1.41 (d, J=6.9 Hz, 6H); MS (ES+): 486.2 (M+23); (ES−): 484.6 (M−1); Analysis calculated for C₂₆H₂₇N₇O₃·0.85HCl·2H₂O: C, 56.51; H, 5.81; Cl, 5.45; N, 17.74; Found: C, 56.84; H, 5.74; Cl, 5.68; N, 17.65.

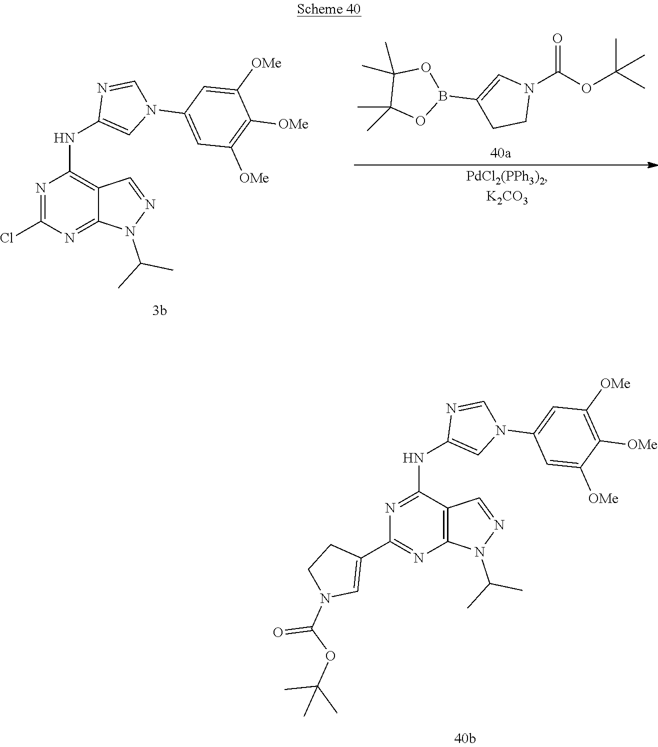 [SMILES: COc1cc(-n2cnc(Nc3nc(Cl)nc4c3cnn4C(C)C)c2)cc(OC)c1OC ; CC(C)(C)OC(=O)N1C=C(B2OC(C)(C)C(C)(C)O2)CC1 ; COc1cc(-n2cnc(Nc3nc(C4=CN(C(=O)OC(C)(C)C)CC4)nc4c3cnn4C(C)C)c2)cc(OC)c1OC]

### Preparation of tert-butyl 4-(1-isopropyl-4-((1-(3,4,5-trimethoxyphenyl)-1H-imidazol-4-yl)amino)-1H-pyrazolo[3,4-d]pyrimidin-6-yl)-2,3-dihydro-1H-pyrrole-1-carboxylate (40b)

Compound 40b was prepared according to the procedure reported in step-2 of scheme 3, from 6-chloro-1-isopropyl-N-(1-(3,4,5-trimethoxyphenyl)-1H-imidazol-4-yl)-1H-pyrazolo[3,4-d]pyrimidin-4-amine (3b) (0.75 g, 1.68 mmol) in 1,4-dioxane (37.5 mL) using tert-butyl 4-(4,4,5,5-tetramethyl-1,3,2-dioxaborolan-2-yl)-2,3-dihydro-1H-pyrrole-1-carboxylate (40a) (0.623 g, 2.11 mmol), a solution of potassium carbonate (0.7 g, 5.06 mmol) in water (7.5 mL), bis(triphenylphosphine)palladium(ii) chloride (0.237 g, 0.337 mmol) and heating at 100° C. for 6 h under argon. This gave after work up and purification using flash column chromatography [silica gel (24 g), eluting with 10% MeOH in DCM] followed by purification using reverse phase column chromatography [C18 (430 g), eluting with ACN in water (containing 0.1% HCl) from 0-100%] tert-butyl 4-(1-isopropyl-4-((1-(3,4,5-trimethoxyphenyl)-1H-imidazol-4-yl)amino)-1H-pyrazolo[3,4-d]pyrimidin-6-yl)-2,3-dihydro-1H-pyrrole-1-carboxylate (40b) HCl salt as a white solid; ¹H NMR (300 MHz, DMSO-d₆) δ 10.90 (s, 1H), 8.49-8.30 (m, 2H), 8.01 (d, J=1.6 Hz, 1H), 6.96 (s, 2H), 5.02 (p, J=6.6 Hz, 1H), 3.95-3.81 (m, 8H), 3.70 (s, 3H), 3.14 (s, 2H), 1.53-1.27 (m, 15H); MS (ES+): 577.20 (M+1); (ES−): 575.10 (M−1).

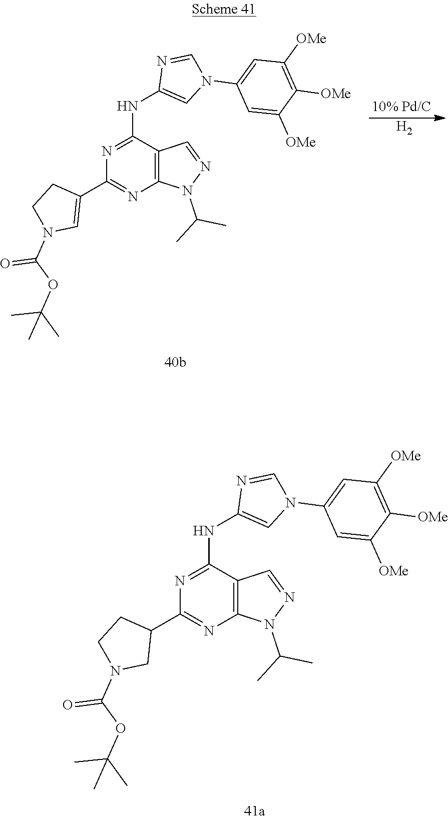 [SMILES: COc1cc(-n2cnc(Nc3nc(C4=CN(C(=O)OC(C)(C)C)CC4)nc4c3cnn4C(C)C)c2)cc(OC)c1OC ; "H2" ; COc1cc(-n2cnc(Nc3nc(C4CCN(C(=O)OC(C)(C)C)C4)nc4c3cnn4C(C)C)c2)cc(OC)c1OC]

### Preparation of tert-butyl 3-(1-isopropyl-4-((1-(3,4,5-trimethoxyphenyl)-1H-imidazol-4-yl)amino)-1H-pyrazolo[3,4-d]pyrimidin-6-yl)pyrrolidine-1-carboxylate (41a)

To a stirred solution of tert-butyl 4-(1-isopropyl-4-((1-(3,4,5-trimethoxyphenyl)-1H-imidazol-4-yl)amino)-1H-pyrazolo[3,4-d]pyrimidin-6-yl)-2,3-dihydro-1H-pyrrole-1-carboxylate (40b) (0.30 g, 0.52 mmol) in MeOH (15 mL) and EtOH (15 mL) was added 10% Pd/C (0.022 g, 0.20 mmol) and stirred at RT for 48 h under a H₂ atmosphere. The reaction mixture was filtered through a pad of Celite, washed with MeOH (30 mL), and concentrated in vacuum. The residue obtained was purified using column chromatography [silica gel, eluting with 3% MeOH in DCM] followed by purification using reverse phase column chromatography [C-18 column (100 g), eluting with ACN in water (containing 0.1% HCl) from 0-100%] to afford tert-butyl 3-(1-isopropyl-4-((1-(3,4,5-trimethoxyphenyl)-1H-imidazol-4-yl)amino)-1H-pyrazolo[3,4-d]pyrimidin-6-yl)pyrrolidine-1-carboxylate (41a) (10 mg, 25% yield) HCl salt as a white solid; ¹H NMR (300 MHz, DMSO-d₆) δ 11.03 (s, 1H), 8.40 (s, 1H), 8.34 (s, 1H), 8.02 (s, 1H), 6.95 (s, 2H), 5.02 (p, J=6.6 Hz, 1H), 3.88 (s, 6H), 3.84-3.71 (m, 1H), 3.69 (s, 3H), 3.64 (m, 3H), 3.35 (s, 1H), 2.31 (m, 2H), 1.46 (d, J=6.7 Hz, 6H), 1.34 (2d, 9H); MS (ES+): 579.20 (M+1).

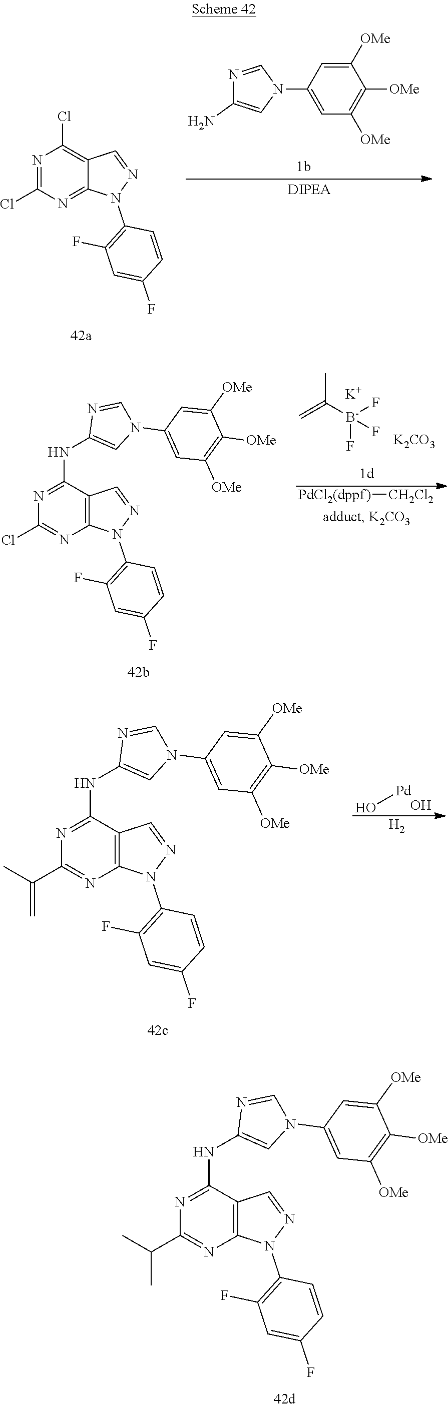

### Preparation of 1-(2,4-difluorophenyl)-6-isopropyl-N-(1-(3,4,5-trimethoxyphenyl)-1H-imidazol-4-yl)-1H-pyrazolo[3,4-d]pyrimidin-4-amine (42d)

### Step-1: Preparation of 6-chloro-1-(2,4-difluorophenyl)-N-(1-(3,4,5-trimethoxyphenyl)-1H-imidazol-4-yl)-1H-pyrazolo[3,4-d]pyrimidin-4-amine (42b)

Compound 42b was prepared according to the procedure reported in step-1 of scheme 1, from 4,6-dichloro-1-(2,4-difluorophenyl)-1H-pyrazolo[3,4-d]pyrimidine (42a) (536 mg, 1.893 mmol; CAS #1260764-81-5) in 2-propanol (20 mL) using DIPEA (0.992 mL, 5.68 mmol), 1-(3,4,5-trimethoxyphenyl)-1H-imidazol-4-amine (1b) (496 mg, 1.988 mmol) and refluxing for 2 h. This gave after work up 6-chloro-1-(2,4-difluorophenyl)-N-(1-(3,4,5-trimethoxyphenyl)-1H-imidazol-4-yl)-1H-pyrazolo[3,4-d]pyrimidin-4-amine (42b) (856 mg, 91% yield) as a brown solid; MS (ES+): 514.10 (M+1).

### Step-2: Preparation of 1-(2,4-difluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (42c)

Compound 42c was prepared according to the procedure reported in step-1 of scheme 1, from 6-chloro-1-(2,4-difluorophenyl)-N-(1-(3,4,5-trimethoxyphenyl)-1H-imidazol-4-yl)-1H-pyrazolo[3,4-d]pyrimidin-4-amine (42b) (500 mg, 0.973 mmol) in dioxane/H₂O (5 mL, ratio: 4:1) using potassium isopropenyltrifluoroborate (1d)(252 mg, 1.703 mmol), potassium carbonate (336 mg, 2.432 mmol), PdCl₂(dppf)-CH₂Cl₂ adduct (159 mg, 0.195 mmol) and heating for 1 h at 150° C. in a microwave. This gave after work up 1-(2,4-difluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (42c) (74 mg, 15% yield); ¹H NMR (300 MHz, DMSO-d₆) δ 11.13 (s, 1H), 8.72 (s, 1H), 8.27 (d, J=1.5 Hz, 1H), 8.13 (s, 1H), 7.79 (td, J=8.7, 6.0 Hz, 1H), 7.69-7.53 (m, 1H), 7.42-7.27 (m, 1H), 6.96 (s, 2H), 6.46-6.32 (m, 1H), 5.55 (s, 1H), 3.88 (s, 6H), 3.70 (s, 3H), 2.25 (s, 3H); ¹⁹F NMR (282 MHz, DMSO-d₆) δ −108.62, −116.05; MS (ES+): 520.10 (M+1).

### Step-3: Preparation of 1-(2,4-difluorophenyl)-6-isopropyl-N-(1-(3,4,5-trimethoxyphenyl)-1H-imidazol-4-yl)-1H-pyrazolo[3,4-d]pyrimidin-4-amine (42d)

Compound 42d was prepared according to the procedure reported in step-3 of scheme 1, from 1-(2,4-difluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (42c) (74 mg, 0.142 mmol) in MeOH (11 mL) using palladium hydroxide on carbon, 20 wt. % loading (dry basis), matrix carbon, wet support (22.0 mg, 0.031 mmol) and stirring overnight at RT under a H₂ atmosphere. This gave after work up and purification using flash column chromatography [silica gel (12 g), eluting with MeOH in DCM from 0-40%] followed by purification using reverse phase column chromatography [C18 column, eluting with ACN in water (containing 0.1% HCl) from 0-100%] 1-(2,4-difluorophenyl)-6-isopropyl-N-(1-(3,4,5-trimethoxyphenyl)-1H-imidazol-4-yl)-1H-pyrazolo[3,4-d]pyrimidin-4-amine (42d) (3 mg, 4% yield) HCl salt as a white solid; ¹H NMR (300 MHz, DMSO-d₆) δ 11.11 (s, 1H, D₂O exchangeable), 8.67 (s, 1H), 8.27 (s, 1H), 8.17 (s, 1H), 7.82-7.70 (m, 1H), 7.68-7.53 (m, 1H), 7.44-7.22 (m, 1H), 6.94 (s, 21H), 3.87 (s, 6H), 3.70 (s, 3H), 3.10-2.94 (m, 1H), 1.33 (d, J=6.8 Hz, 6H); ¹⁹F NMR (282 MHz, DMSO-d₆) δ −108.40, −116.10; MS (ES+): 522.2 (M+1).

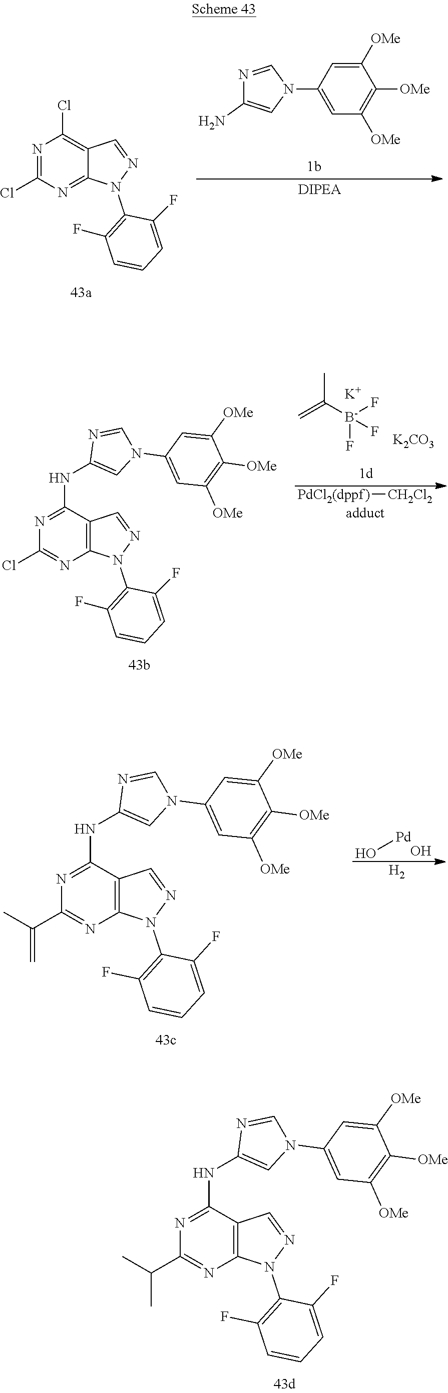

### Preparation of 1-(2,6-difluorophenyl)-6-isopropyl-N-(1-(3,4,5-trimethoxyphenyl)-1H-imidazol-4-yl)-1H-pyrazolo[3,4-d]pyrimidin-4-amine (43d)

### Step-1: Preparation of 6-chloro-1-(2,6-difluorophenyl)-N-(1-(3,4,5-trimethoxyphenyl)-1H-imidazol-4-yl)-1H-pyrazolo[3,4-d]pyrimidin-4-amine (43b)

Compound 43b was prepared according to the procedure reported in step-1 of scheme 1, from 4,6-dichloro-1-(2,6-difluorophenyl)-1H-pyrazolo[3,4-d]pyrimidine (43a) (440 mg, 1.554 mmol; CAS #2060595-18-6) in 2-propanol (20 mL) using DIPEA (0.814 mL, 4.66 mmol), 1-(3,4,5-trimethoxyphenyl)-1H-imidazol-4-amine (1b) (407 mg, 1.632 mmol) and refluxing for 2 h. This gave after work up 6-chloro-1-(2,6-difluorophenyl)-N-(1-(3,4,5-trimethoxyphenyl)-1H-imidazol-4-yl)-1H-pyrazolo[3,4-d]pyrimidin-4-amine (43b) (378 mg, 49% yield) as a yellow solid; MS (ES+): 514.10 (M+1).

### Step-2: Preparation of 1-(2,6-difluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (43c)

Compound 43c was prepared according to the procedure reported in step-1 of scheme 1, from 6-chloro-1-(2,6-difluorophenyl)-N-(1-(3,4,5-trimethoxyphenyl)-1H-imidazol-4-yl)-1H-pyrazolo[3,4-d]pyrimidin-4-amine (43b) (500 mg, 0.973 mmol) in dioxane/H₂O (5 mL, ratio: 4:1) using potassium isopropenyltrifluoroborate (1d)(252 mg, 1.703 mmol), potassium carbonate (336 mg, 2.432 mmol), PdCl₂(dppf)-CH₂Cl₂ adduct (159 mg, 0.195 mmol) and heating for 1 h at 150° C. in a microwave. This gave after work up and purification by flash column chromatography [silica gel (24 g), eluting with DMA-80 in DCM from 0-80%] 1-(2,6-difluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (43c) (275 mg, 54% yield); ¹H NMR (300 MHz, DMSO-d₆) δ 11.18 (s, 1H), 8.77 (s, 1H), 8.28 (d, J=1.6 Hz, 1H), 8.13 (s, 1H), 7.77-7.68 (m, 1H), 7.45 (t, J=8.3 Hz, 2H), 6.97 (s, 2H), 6.36 (s, 1H), 5.54 (s, 1H), 3.88 (s, 6H), 3.70 (s, 3H), 2.23 (s, 3H); ¹⁹F NMR (282 MHz, DMSO-d₆) δ −118.76; MS (ES+): 520.20 (M+1).

### Step-3: Preparation of 1-(2,6-difluorophenyl)-6-isopropyl-N-(1-(3,4,5-trimethoxyphenyl)-1H-imidazol-4-yl)-1H-pyrazolo[3,4-d]pyrimidin-4-amine (43d)

Compound 43d was prepared according to the procedure reported in step-3 of scheme 1, from 1-(2,6-difluorophenyl)-6-(prop-1-en-2-yl)-N-(1-(3,4,5-trimethoxyphenyl)-1H-imidazol-4-yl)-1H-pyrazolo[3,4-d]pyrimidin-4-amine (43c) (270 mg, 0.52 mmol) in MeOH (11 mL) using palladium hydroxide on carbon, 20 wt. % loading (dry basis), matrix carbon, wet support (80 mg, 0.114 mmol) and stirring overnight at RT under a H₂ atmosphere. This gave after work up and purification using flash column chromatography [silica gel (40 g), eluting with MeOH in DCM from 0-15%] followed by purification using reverse phase column chromatography [C18 column, eluting with ACN in water (containing 0.1% HCl) from 0-100%] 1-(2,6-difluorophenyl)-6-isopropyl-N-(1-(3,4,5-trimethoxyphenyl)-1H-imidazol-4-yl)-1H-pyrazolo[3,4-d]pyrimidin-4-amine (43d) (35 mg, 13% yield) HCl salt as a white solid; ¹H NMR (300 MHz, DMSO-d₆) δ 11.23 (s, 1H, D₂O exchangeable), 8.72 (s, 1H), 8.37 (s, 1H), 8.17 (s, 1H), 7.81-7.63 (m, 1H), 7.51-7.35 (m, 2H), 6.97 (s, 2H), 3.87 (s, 6H), 3.70 (s, 3H), 3.10-2.95 (m, 1H), 1.31 (d, J=6.8 Hz, 6H); ¹⁹F NMR (282 MHz, DMSO-d₆) δ −118.80; MS (ES+): 522.20 (M+1).

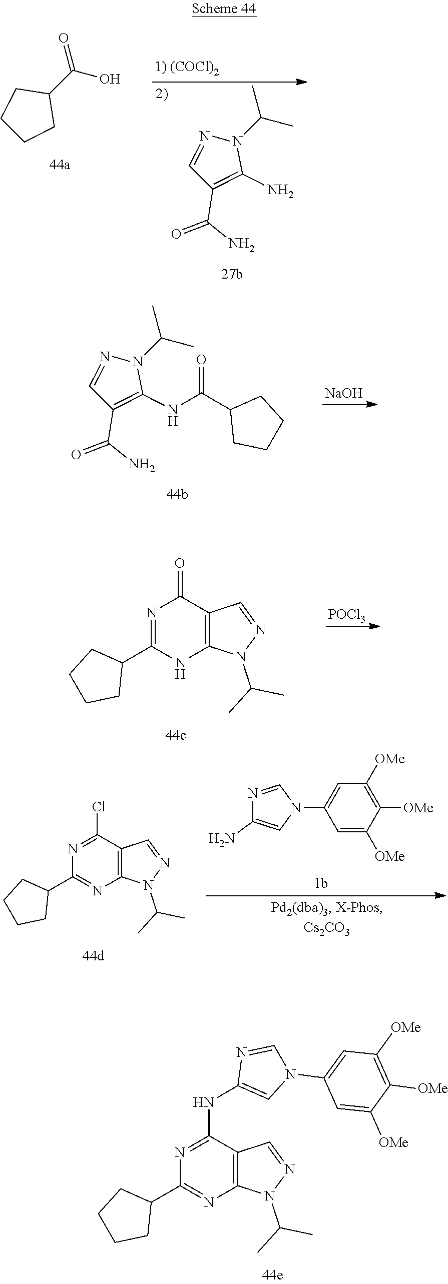

### Preparation of 6-cyclopentyl-1-isopropyl-N-(1-(3,4,5-trimethoxyphenyl)-1H-imidazol-4-yl)-1H-pyrazolo[3,4-d]pyrimidin-4-amine (44e)

### Step-1: Preparation of 5-(cyclopentanecarboxamido)-1-isopropyl-1H-pyrazole-4-carboxamide (44b)

Compound 44b was prepared according to the procedure reported in step-1 of scheme 27, from cyclopentanecarboxylic acid (44a) (1.0 g, 8.76 mmol; CAS #3400-45-1) in DCM (20.0 mL) using oxalyl chloride (3.33 g, 26.28 mmol) and 5-amino-1-isopropyl-1H-pyrazole-4-carboxamide (27b) (1.05 g, 6.24 mmol) in 1,4-dioxane (30 mL) and stirring at RT for 12 h. This gave after work up and purification using column chromatography [silica gel, eluting with MeOH in DCM from 0-5%] 5-(cyclopentanecarboxamido)-1-isopropyl-1H-pyrazole-4-carboxamide (44b) (400 mg, 24% yield) as an off-white solid; ¹H NMR (300 MHz, DMSO-d₆) δ 11.97 (s, 1H), 7.97 (s, 1H), 5.08-4.70 (m, 1H), 3.14-3.00 (m, 1H), 2.05-1.50 (m, 6H), 1.43 (d, J=6.7 Hz, 6H), 1.35-1.07 (m, 2H).

### Step-2: Preparation of 6-cyclopentyl-1-isopropyl-1H-pyrazolo[3,4-d]pyrimidin-4 (7H)-one (44c)

Compound 44c was prepared according to the procedure reported in step-2 of scheme 27, from 5-(cyclopentanecarboxamido)-1-isopropyl-1H-pyrazole-4-carboxamide (44b) (400 mg, 1.51 mmol) using a solution of NaOH (2N) (0.6 g, 15.13 mmol) and heating at 70° C. for 0.5 h. This gave after work up 6-cyclopentyl-1-isopropyl-1H-pyrazolo[3,4-d]pyrimidin-4 (7H)-one (44c) (350 mg, 94% yield) as an off white solid, ¹H NMR (300 MHz, DMSO-d₆) δ 11.97 (s, 1H), 7.97 (s, 1H), 5.06-4.73 (m, 1H), 3.17-2.99 (m, 1H), 2.09-1.51 (m, 6H), 1.43 (d, J=6.7 Hz, 6H), 1.23 (m, 2H).

### Step-3: Preparation of 4-chloro-6-cyclopentyl-1-isopropyl-1H-pyrazolo[3,4-d]pyrimidine (44d)

Compound 44d was prepared according to the procedure reported in step-3 of scheme 27, from 6-cyclopentyl-1-isopropyl-1H-pyrazolo[3,4-d]pyrimidin-4 (7H)-one (44c) (0.8 g, 3.25 mmol) using POCl₃ (28.38 g, 185.13 mmol) and heating at 100° C. for 1 h. This gave after work up and purification using column chromatography [silica gel, eluting with 10% MeOH in DCM] 4-chloro-6-cyclopentyl-1-isopropyl-1H-pyrazolo[3,4-d]pyrimidine (44d) (600 mg, 70% yield) as an oil; ¹H NMR (300 MHz, DMSO-d₆) δ 8.36 (s, 1H), 5.21-4.96 (m, 1H), 3.45-3.37 (m, 1H), 2.15-1.59 (m, 6H), 1.50 (d, J=6.7 Hz, 6H), 1.35-1.11 (m, 2H).

### Step-4: Preparation of 6-cyclopentyl-1-isopropyl-N-(1-(3,4,5-trimethoxyphenyl)-1H-imidazol-4-yl)-1H-pyrazolo[3,4-d]pyrimidin-4-amine (44e)

Compound 44e was prepared according to the procedure reported in step-4 of scheme 27, from 4-chloro-6-cyclopentyl-1-isopropyl-1H-pyrazolo[3,4-d]pyrimidine (44d) (0.6 g, 2.27 mmol) in 1,4-dioxane (18 mL) using 1-(3,4,5-trimethoxyphenyl)-1H-imidazol-4-amine (1b) (0.734 g, 2.94 mmol), Pd₂(dba)₃ (0.41 g, 0.453 mmol), X-phos (0.42 g, 0.90 mmol), Cs₂CO3 (2.21 g, 6.79 mmol) and heating at 120° C. for 4 h. This gave after work up and purification using column chromatography [silica gel, eluting with 10% MeOH in DCM] 6-cyclopentyl-1-isopropyl-N-(1-(3,4,5-trimethoxyphenyl)-1H-imidazol-4-yl)-1H-pyrazolo[3,4-d]pyrimidin-4-amine (44e) (0.32 g, 30% yield) as an off white solid; ¹H NMR (300 MHz, DMSO-d₆) δ 10.81 (s, 1H, D₂O exchangeable), 8.38 (s, 1H), 8.21 (d, J=1.6 Hz, 1H), 8.06 (s, 1H), 6.91 (s, 2H), 5.13-4.88 (m, 1H), 3.88 (s, 6H), 3.69 (s, 3H), 3.33-3.18 (m, 1H), 2.24-1.91 (m, 4H), 1.90-1.56 (m, 4H), 1.45 (d, J=6.6 Hz, 6H); Analysis calculated for C₂₅H₃₁N₇O₃: C, 62.88; H, 6.54; N, 20.53; Found: C, 62.95; H, 6.58; N, 20.18.

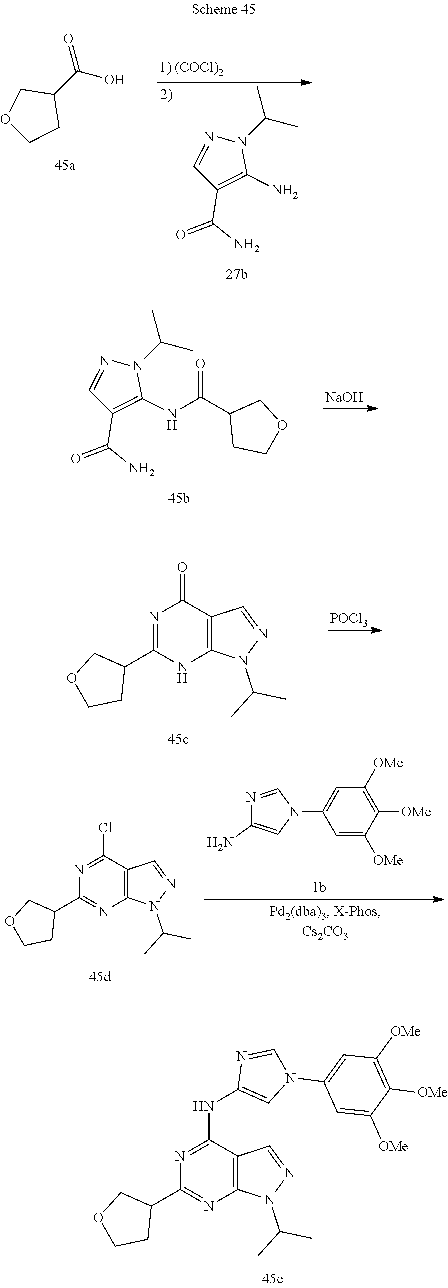

### Preparation of 1-isopropyl-6-(tetrahydrofuran-3-yl)-N-(1-(3,4,5-trimethoxyphenyl)-1H-imidazol-4-yl)-1H-pyrazolo[3,4-d]pyrimidin-4-amine (45e)

### Step-1: Preparation of 1-isopropyl-5-(tetrahydrofuran-3-carboxamido)-1H-pyrazole-4-carboxamide (45b)

Compound 45b was prepared according to the procedure reported in step-1 of scheme 27, from tetrahydrofuran-3-carboxylic acid (45a) (1.0 g, 8.61 mmol; CAS #89364-31-8) in DCM (20.0 mL) using oxalyl chloride (3.27 g, 25.84 mmol), 5-amino-1-isopropyl-1H-pyrazole-4-carboxamide (27b) (1.02 g, 6.06 mmol) in 1,4-dioxane (30 mL) and stirring at RT for 12 h. This gave after work up and purification using column chromatography [silica gel, eluting with MeOH in DCM from 0-5%] 1-isopropyl-5-(tetrahydrofuran-3-carboxamido)-1H-pyrazole-4-carboxamide (45b) (850 mg, 53% yield) as an off-white solid; ¹H NMR (300 MHz, DMSO-d₆) δ 9.83 (s, 1H), 7.89 (s, 1H), 7.33 (s, 1H), 6.97 (s, 1H), 4.42-4.16 (m, 1H), 4.09-3.79 (m, 2H), 3.79-3.61 (m, 2H), 3.30-3.17 (m, 1H), 2.21-2.03 (m, 2H), 1.31 (d, J=6.6 Hz, 6H).

### Step-2: Preparation of 1-isopropyl-6-(tetrahydrofuran-3-yl)-1H-pyrazolo[3,4-d]pyrimidin-4 (7H)-one (45c)

Compound 45c was prepared according to the procedure reported in step-2 of scheme 27, from 1-isopropyl-5-(tetrahydrofuran-3-carboxamido)-1H-pyrazole-4-carboxamide (45b) (800 mg, 3 mmol) using a solution of NaOH (2N) (1.32 g, 15.13 mmol) and heating at 70° C. for 0.5 h. This gave after work up 1-isopropyl-6-(tetrahydrofuran-3-yl)-1H-pyrazolo[3,4-d]pyrimidin-4 (7H)-one (45c) (650 mg, 87% yield) as an off white solid; ¹H NMR (300 MHz, DMSO-d₆) δ 12.10 (s, 1H), 8.00 (s, 1H), 5.12-4.75 (m, 1H), 4.11-4.00 (m, 1H), 3.95-3.83 (m, 2H), 3.82-3.71 (m, 1H), 3.61-3.39 (m, 1H), 2.36-2.07 (m, 2H), 1.44 (d, J=6.7 Hz, 6H).

### Step-3: Preparation of 4-chloro-1-isopropyl-6-(tetrahydrofuran-3-yl)-1H-pyrazolo[3,4-d]pyrimidine (45d)

Compound 45d was prepared according to the procedure reported in step-3 of scheme 27, from 1-isopropyl-6-(tetrahydrofuran-3-yl)-1H-pyrazolo[3,4-d]pyrimidin-4 (7H)-one (45c) (0.6 g, 2.42 mmol) using POCl₃ (21.12 g, 137.74 mmol) and heating at 100° C. for 1 h. This gave after work up and purification using column chromatography [silica gel, eluting with MeOH in DCM from 0-10%] 4-chloro-1-isopropyl-6-(tetrahydrofuran-3-yl)-1H-pyrazolo[3,4-d]pyrimidine (45d) (250 mg, 39% yield) as an oil; ¹H NMR (300 MHz, DMSO-d₆) δ 8.40 (s, 1H), 5.32-4.95 (m, 1H), 4.13 (t, J=8.1 Hz, 1H), 3.98-3.88 (m, 2H), 3.88-3.71 (m, 2H), 2.39-2.23 (m, 2H), 1.50 (d, J=6.7 Hz, 6H).

### Step-4: Preparation of 1-isopropyl-6-(tetrahydrofuran-3-yl)-N-(1-(3,4,5-trimethoxyphenyl)-1H-imidazol-4-yl)-1H-pyrazolo[3,4-d]pyrimidin-4-amine (45e)

Compound 45e was prepared according to the procedure reported in step-4 of scheme 27, from 4-chloro-1-isopropyl-6-(tetrahydrofuran-3-yl)-1H-pyrazolo[3,4-d]pyrimidine (45d) (0.25 g, 0.94 mmol) in 1,4-dioxane (7.5 mL) using 1-(3,4,5-trimethoxyphenyl)-1H-imidazol-4-amine (1b) (0.30 g, 1.20 mmol), Pd₂(dba)₃ (0.17 g, 0.18 mmol), X-phos (0.17 g, 0.35 mmol), Cs₂CO₃ (0.91 g, 2.81 mmol) and heating at 120° C. for 4 h. his gave after work up and purification using column chromatography [silica gel, eluting with MeOH in DCM from 0-10%] 1-isopropyl-6-(tetrahydrofuran-3-yl)-N-(1-(3,4,5-trimethoxyphenyl)-1H-imidazol-4-yl)-1H-pyrazolo[3,4-d]pyrimidin-4-amine (45e) (0.115 g, 26% yield) as an off white solid; ¹H NMR (300 MHz, DMSO-d₆) δ 10.92 (s, 1H, D₂O exchangeable), 8.40 (s, 1H), 8.32-8.13 (m, 2H), 6.97 (s, 2H), 5.14-4.93 (m, 1H), 4.31-4.16 (m, 1H), 4.15-3.96 (m, 2H), 3.90 (s, 6H), 3.89-3.76 (m, 1H), 3.70 (s, 3H), 3.69-3.56 (m, 1H), 2.47-2.31 (m, 1H), 2.31-2.10 (m, 1H), 1.46 (dd, J=6.6, 3.0 Hz, 6H); Analysis calculated for: C₂₄H₂₉N₇O₄: C, 60.11; H, 6.10; N, 20.45; Found: C, 60.03; H, 6.10; N, 20.14.

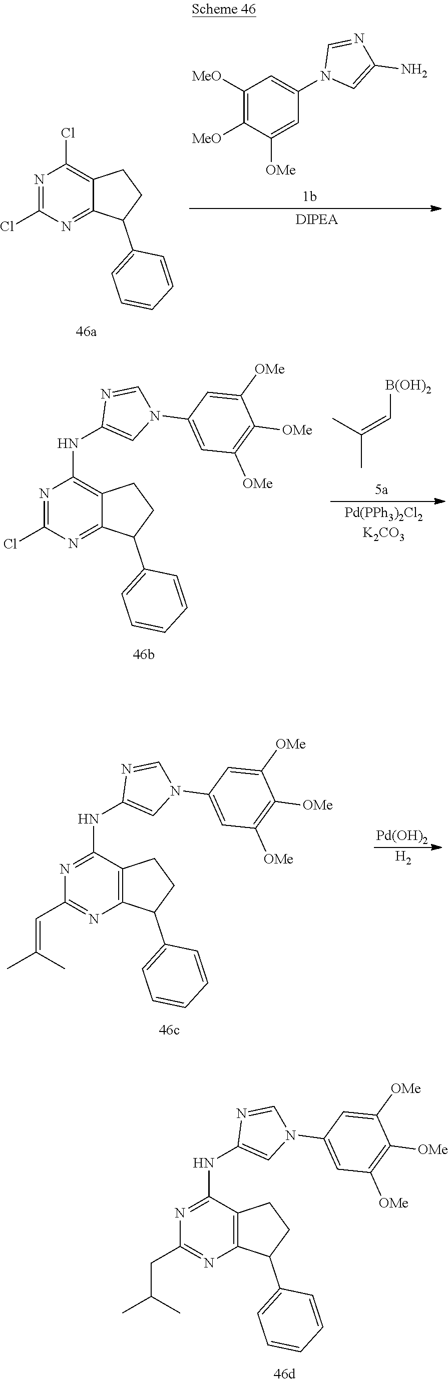

### Preparation of 2-isobutyl-7-phenyl-N-(1-(3,4,5-trimethoxyphenyl)-1H-imidazol-4-yl)-6,7-dihydro-5H-cyclopenta[d]pyrimidin-4-amine (46d)

### Step-1: Preparation of 2-chloro-7-phenyl-N-(1-(3,4,5-trimethoxyphenyl)-1H-imidazol-4-yl)-6,7-dihydro-5H-cyclopenta[d]pyrimidin-4-amine (46b)

Compound 46b was prepared according to the procedure reported in step-1 of scheme 1, from 2,4-dichloro-7-phenyl-6,7-dihydro-5H-cyclopenta[d]pyrimidine (46a) (1.1 g, 4.148 mmol; CAS #1263868-24-1) in 2-propanol (16.5 mL) using DIPEA (2.1 mL, 12.444 mmol), 1-(3,4,5-trimethoxyphenyl)-1H-imidazol-4-amine (1b) (1.34 g, 5.39 mmol) and heating at 82° C. for 15 h. This gave after work up 2-chloro-7-phenyl-N-(1-(3,4,5-trimethoxyphenyl)-1H-imidazol-4-yl)-6,7-dihydro-5H-cyclopenta[d]pyrimidin-4-amine (46b) (1.3 g, 63% yield) as an off white solid; ¹H NMR (300 MHz, DMSO-d₆) δ 10.06 (s, 1H), 8.16 (d, J=1.6 Hz, 1H), 7.79 (d, J=1.6 Hz, 1H), 7.37-7.18 (m, 3H), 7.21-7.12 (m, 2H), 6.90 (s, 2H), 4.27 (t, J=8.0 Hz, 1H), 3.87 (s, 6H), 3.69 (s, 3H), 3.03-2.93 (m, 1H), 2.88-2.77 (m, 1H), 2.67-2.54 (m, 1H), 2.11-1.95 (m, 1H).

### Step-2: Preparation of 2-(2-methylprop-1-en-1-yl)-7-phenyl-N-(1-(3,4,5-trimethoxyphenyl)-1H-imidazol-4-yl)-6,7-dihydro-5H-cyclopenta[d]pyrimidin-4-amine (46c)

Compound 46c was prepared according to the procedure reported in step-2 of scheme 3, from 2-chloro-7-phenyl-N-(1-(3,4,5-trimethoxyphenyl)-1H-imidazol-4-yl)-6,7-dihydro-5H-cyclopenta[d]pyrimidin-4-amine (46b) (0.7 g, 1.46 mmol) in 1,4-dioxane (12 mL) using (2-methylprop-1-en-1-yl)boronic acid (5a) (0.22 g, 2.196 mmol), a solution of potassium carbonate (0.605 g, 4.38 mmol) in water (1.4 mL), bis(triphenylphosphine)palladium(II) chloride (0.20 g, 0.292 mmol) and heating at 120° C. for 4 h under argon. This gave after work up and purification using flash column chromatography [silica gel, eluting with Methanol in ethyl acetate from 0 to 2%] 2-(2-methylprop-1-en-1-yl)-7-phenyl-N-(1-(3,4,5-trimethoxyphenyl)-1H-imidazol-4-yl)-6,7-dihydro-5H-cyclopenta[d]pyrimidin-4-amine (46c) (0.385 g, 53% yield) as a white solid; ¹H NMR (300 MHz, DMSO-d₆) δ 9.36 (s, 1H), 8.15 (d, J=1.6 Hz, 1H), 7.90 (d, J=1.6 Hz, 1H), 7.36-7.24 (m, 2H), 7.24-7.10 (m, 3H), 6.89 (s, 2H), 6.20 (s, 1H), 4.24 (t, J=7.9 Hz, 1H), 3.88 (s, 6H), 3.69 (s, 3H), 3.11-2.93 (m, 1H), 2.94-2.75 (m, 1H), 2.63-2.53 (m, 1H), 2.17 (s, 3H), 2.06-1.94 (m, 1H), 1.85 (s, 3H).

### Step-3: Preparation of 2-isobutyl-7-phenyl-N-(1-(3,4,5-trimethoxyphenyl)-1H-imidazol-4-yl)-6,7-dihydro-5H-cyclopenta[d]pyrimidin-4-amine (46d)

Compound 46d was prepared according to the procedure reported in step-3 of scheme 1, from 2-(2-methylprop-1-en-1-yl)-7-phenyl-N-(1-(3,4,5-trimethoxyphenyl)-1H-imidazol-4-yl)-6,7-dihydro-5H-cyclopenta[d]pyrimidin-4-amine (46c) (0.44 g, 0.884 mmol) in MeOH (15 mL) using Pd(OH)₂ (20% in H₂O) (0.247 g, 0.176 mmol) and stirring for 3 days at RT under a H₂ atmosphere. This gave after work up and crystallization (using IPA) a brown solid, which was further purified using reverse phase column chromatography [C18 column (100 g), eluting with ACN in water (containing 0.1% HCl) from 0-100%]2-isobutyl-7-phenyl-N-(1-(3,4,5-trimethoxyphenyl)-1H-imidazol-4-yl)-6,7-dihydro-5H-cyclopenta[d]pyrimidin-4-amine (46d) (0.025 g, 50%) HCl salt as a white solid; ¹H NMR (300 MHz, DMSO-d₆) δ 14.31 (s, 1H), 11.50 (s, 1H), 8.36 (d, J=1.6 Hz, 1H), 8.10 (d, J=1.6 Hz, 1H), 7.45-7.28 (m, 3H), 7.28-7.16 (m, 2H), 6.95 (s, 2H), 4.72-4.52 (m, 1H), 3.89 (s, 6H), 3.70 (s, 3H), 3.23-3.07 (m, 1H), 3.06-2.88 (m, 1H), 2.84-2.68 (m, 3H), 2.37-2.16 (m, 1H), 2.09-1.91 (m, 1H), 0.96 (dd, J=6.6, 1.9 Hz, 6H); MS (ES+): 500.20 (M+1); MS (ES−): 498.20 (M−1).

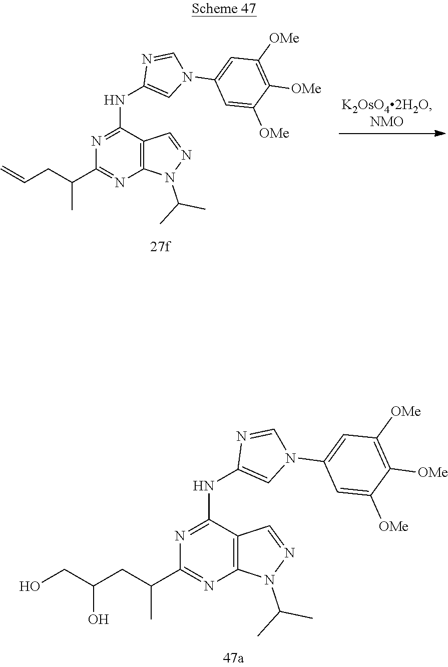 [SMILES: C=CCC(C)c1nc(Nc2cn(-c3cc(OC)c(OC)c(OC)c3)cn2)c2cnn(C(C)C)c2n1 ; COc1cc(-n2cnc(Nc3nc(C(C)CC(O)CO)nc4c3cnn4C(C)C)c2)cc(OC)c1OC]

### Preparation of 4-(1-isopropyl-4-((1-(3,4,5-trimethoxyphenyl)-1H-imidazol-4-yl)amino)-1H-pyrazolo[3,4-d]pyrimidin-6-yl)pentane-1,2-diol (47a)

To a stirred solution of 1-isopropyl-6-(pent-4-en-2-yl)-N-(1-(3,4,5-trimethoxyphenyl)-1H-imidazol-4-yl)-1H-pyrazolo[3,4-d]pyrimidin-4-amine) (27f) (4.0 g, 8.38 mmol) in acetone (240 mL) and water (40 mL) was added 4-methyl morpholine N-oxide (NMO) (10.0 g, 42.68 mmol), K₂OsO₄·2H₂O (2.0 g, 5.43 mmol) and stirred at RT for 15 h. Reaction was quenched with sodium sulfite (47.2 g), stirred for 45 min, filtered through a pad of Celite and washed with acetone (200 mL). The filtrate was concentrated and diluted with water (200 mL) and extracted with EtOAc (4×200 mL). The combined organics were washed with brine (200 mL), dried, filtered and concentrated in vacuum. The residue obtained was purified using column chromatography [silica gel, eluting with MeOH in DCM from 0% to 10%] to give 4-(1-isopropyl-4-((1-(3,4,5-trimethoxyphenyl)-1H-imidazol-4-yl)amino)-1H-pyrazolo[3,4-d]pyrimidin-6-yl)pentane-1,2-diol (47a) (1.05 g, 25%) as white solid; ¹H NMR (300 MHz, DMSO-d₆) δ 10.88-10.69 (m, 1H), 8.37 (s, 1H), 8.21 (s, 1H), 8.14 (d, J=8.2 Hz, 1H), 6.94 (d, J=6.2 Hz, 2H), 5.11-4.92 (m, 1H), 4.51 (d, J=5.1 Hz, 1H), 4.48-4.34 (m, 2H), 3.88 (s, 6H), 3.69 (s, 3H), 3.31-3.05 (m, 3H), 1.45 (dd, J=6.7, 2.3 Hz, 6H), 1.32 (dd. J=6.9, 4.1 Hz, 3H); MS (ES+): 512.20 (M+1); MS (ES−): 510.20 (M−1).

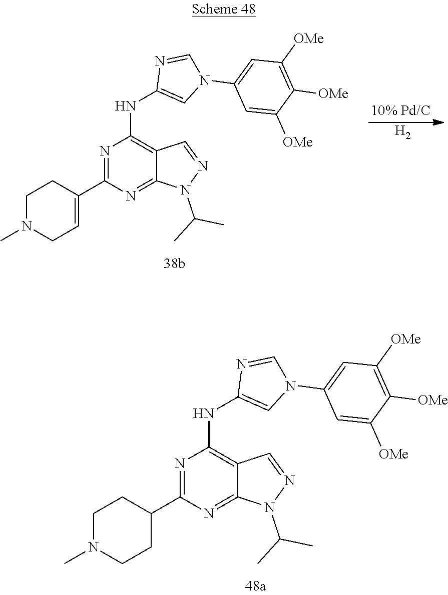 [SMILES: COc1cc(-n2cnc(Nc3nc(C4=CCN(C)CC4)nc4c3cnn4C(C)C)c2)cc(OC)c1OC ; "H2" ; COc1cc(-n2cnc(Nc3nc(C4CCN(C)CC4)nc4c3cnn4C(C)C)c2)cc(OC)c1OC]

### Preparation of 1-isopropyl-6-(1-methylpiperidin-4-yl)-N-(1-(3,4,5-trimethoxyphenyl)-1H-imidazol-4-yl)-1H-pyrazolo[3,4-d]pyrimidin-4-amine (48a)

Compound 48a was prepared according to the procedure reported in step-1 of scheme 1, from 1-isopropyl-6-(1-methyl-1,2,3,6-tetrahydropyridin-4-yl)-N-(1-(3,4,5-trimethoxyphenyl)-1H-imidazol-4-yl)-1H-pyrazolo[3,4-d]pyrimidin-4-amine (38b) (300 mg, 0.59 mmol) in MeOH (18 mL) and EtOH (18 mL) using 10% Pd/C (0.012 g, 0.11 mmol) and stirring at RT for 24 h under a H₂ atmosphere. This gave after work up and purification using column chromatography [silica gel, eluting with 10% MeOH in DCM] followed by purification using reverse phase column chromatography [C-18 column (130 g), eluting with ACN in water (containing 0.1% HCl) from 0-100%] 1-isopropyl-6-(1-methylpiperidin-4-yl)-N-(1-(3,4,5-trimethoxyphenyl)-1H-imidazol-4-yl)-1H-pyrazolo[3,4-d]pyrimidin-4-amine (48a) (0.074 g, 62% yield) HCl salt as a white solid; ¹H NMR (300 MHz, DMSO-d₆) δ 10.95 (s, 1H), 9.84 (s, 1H), 8.42 (s, 1H), 8.28 (s, 1H), 7.99 (d, J=11.6 Hz, 1H), 6.96 (s, 2H), 5.11-4.88 (m, 1H), 3.89 (s, 6H), 3.70 (s, 3H), 3.56-3.42 (m, 2H), 3.19-2.97 (m, 3H), 2.79 (d, J=4.7 Hz, 3H), 2.74-2.64 (m, 1H), 2.43-2.29 (m, 1H), 2.19-1.99 (m, 2H), 1.55-1.35 (m, 6H); MS (ES+): 507.20 (M+1); MS (ES−): 505.20 (M−1); Analysis calculated for C₂₆H₃₄N₈O₃·2HCl·5.75H₂O: C, 45.71; H, 7.01; Cl, 10.38; N, 16.40; Found: C, 45.88; H, 6.78; Cl, 10.05; N, 16.29.

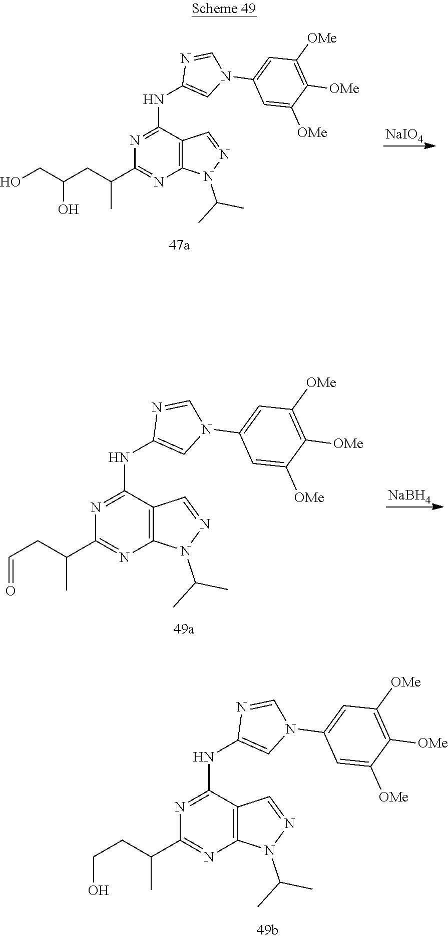

### Preparation of 3-(1-isopropyl-4-((1-(3,4,5-trimethoxyphenyl)-1H-imidazol-4-yl)amino)-1H-pyrazolo[3,4-d]pyrimidin-6-yl)butan-1-ol (49b)

### Step-1: Preparation of 3-(1-isopropyl-4-((1-(3,4,5-trimethoxyphenyl)-1H-imidazol-4-yl)amino)-1H-pyrazolo[3,4-d]pyrimidin-6-yl)butanal (49a)

To a stirred ice-cold solution of 4-(1-isopropyl-4-((1-(3,4,5-trimethoxyphenyl)-1H-imidazol-4-yl)amino)-1H-pyrazolo[3,4-d]pyrimidin-6-yl)pentane-1,2-diol (47a) (0.25 g, 0.49 mmol) in 1,4-dioxane (12.5 mL) was added a solution of saturated aqueous sodium bicarbonate (2.5 mL) and sodium metaperiodate (0.63 g, 2.95 mmol). The reaction mixture was stirred for 6 h, diluted with ethyl acetate (100 mL), filtered through a pad of Celite, washed with ethyl acetate (50 mL) and the filtrate was concentrated to give 3-(1-isopropyl-4-((1-(3,4,5-trimethoxyphenyl)-1H-imidazol-4-yl)amino)-1H-pyrazolo[3,4-d]pyrimidin-6-yl)butanal (49a) (0.23 g), which was used as such for next step; ¹H NMR (300 MHz, DMSO-d₄) δ 10.86 (s, 1H), 9.78 (s, 1H), 8.39 (s, 1H), 8.22 (s, 1H), 8.03 (s, 1H), 6.97 (s, 2H), 5.07-4.90 (m, 2H), 3.88 (s, 6H), 3.70 (s, 3H), 3.08-2.64 (m, 2H), 1.50-1.42 (m, 9H).

### Step-2: Preparation of 3-(1-isopropyl-4-((1-(3,4,5-trimethoxyphenyl)-1H-imidazol-4-yl)amino)-1H-pyrazolo[3,4-d]pyrimidin-6-yl)butan-1-ol (49b)

To a stirred solution of 3-(1-isopropyl-4-((1-(3,4,5-trimethoxyphenyl)-1H-imidazol-4-yl)amino)-1H-pyrazolo[3,4-d]pyrimidin-6-yl)butanal (49a) (0.1 g, 0.21 mmol) in MeOH (5.0 mL) was added at 0° C. sodium borohydride (35 mg, 0.982 mmol) and stirred at RT for 2 h. The reaction was quenched at 0° C. with saturated aqueous NH₄Cl solution (50 mL) and extracted with ethyl acetate (2×100 mL). Combined organics were washed with brine (100 mL), dried, filtered and concentrated in vacuum. The residue obtained was purified using column chromatography [silicagel, eluting with MeOH in DCM from 0% to 3%] to give 3-(1-isopropyl-4-((1-(3,4,5-trimethoxyphenyl)-1H-imidazol-4-yl)amino)-1H-pyrazolo[3,4-d]pyrimidin-6-yl)butan-1-ol (49b) (36 mg, 36% yield) as a brown solid; ¹H NMR (300 MHz, DMSO-d₆) δ 10.84 (s, 1H), 8.38 (s, 1H), 8.23 (s, 1H), 8.14 (s, 1H), 6.93 (s, 2H), 5.13-4.94 (m, 1H), 4.44 (t, J=5.1 Hz, 1H), 3.88 (s, 6H), 3.70 (s, 3H), 3.53-3.38 (m, 2H), 3.16-2.99 (m, 1H), 2.18-2.00 (m, 1H), 1.87-1.68 (m, 1H), 1.46 (d, J=6.7 Hz, 6H), 1.34 (d, J=6.9 Hz, 3H); MS (ES+): 482.20 (M+1); (ES−): 480.10 (M−1).

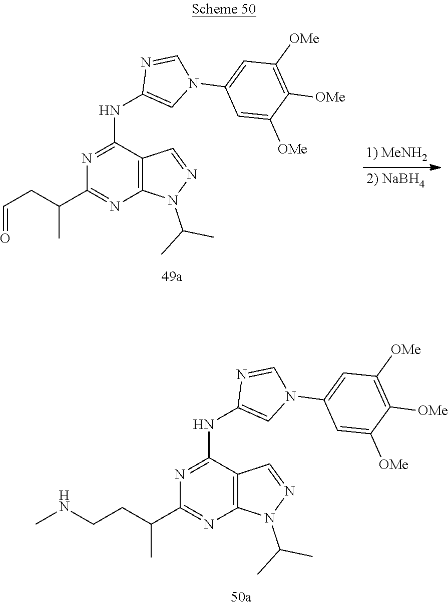 [SMILES: COc1cc(-n2cnc(Nc3nc(C(C)CC=O)nc4c3cnn4C(C)C)c2)cc(OC)c1OC ; CNCCC(C)c1nc(Nc2cn(-c3cc(OC)c(OC)c(OC)c3)cn2)c2cnn(C(C)C)c2n1]

### Preparation of 1-isopropyl-6-(4-(methylamino)butan-2-yl)-N-(1-(3,4,5-trimethoxyphenyl)-1H-imidazol-4-yl)-1H-pyrazolo[3,4-d]pyrimidin-4-amine (50a)

To a stirred solution of 3-(1-isopropyl-4-((1-(3,4,5-trimethoxyphenyl)-1H-imidazol-4-yl)amino)-1H-pyrazolo[3,4-d]pyrimidin-6-yl)butanal (49a) (0.23 g, 0.479 mmol) in MeOH (10.0 mL) was added 7% methyl amine in THF (0.42 mL, 0.958 mmol) and stirred at RT for 2 h. To this mixture cooled to 0° C. was added sodium borohydride (36 mg, 0.958 mmol) and stirred at RT for 2 h. The reaction was quenched with 2N NaOH (50.0 mL) and extracted with ethyl acetate (2×100 mL). Combined organics were washed with brine (100 mL), dried, filtered and concentrated in vacuum and the residue obtained was purified using column chromatography [silica gel, eluting with 20% MeOH in DCM] followed by purification using reverse phase column chromatography [C18 column (50 g), eluting with ACN in water (containing 0.1% HCl) from 0-100%] I-isopropyl-6-(4-(methylamino)butan-2-yl)-N-(1-(3,4,5-trimethoxyphenyl)-1H-imidazol-4-yl)-1H-pyrazolo[3,4-d]pyrimidin-4-amine (50a) (0.013 g, 45% yield) HCl salt as a white solid; ¹H NMR (300 MHz, DMSO-d₆) δ 11.18 (s, 1H), 8.69 (s, 3H), 8.43 (s, 1H), 8.10 (s, 1H), 6.98 (s, 2H), 5.15-4.95 (m, 1H), 3.89 (s, 6H), 3.70 (s, 3H), 3.19-3.00 (m, 1H), 2.98-2.69 (m, 2H), 2.50 (s, 3H), 2.33-2.10 (m, 1H), 2.04-1.86 (m, 1H), 1.46 (dd, J=6.6 Hz, 6H), 1.37 (d, J=6.8 Hz, 3H); MS (ES+): 495.30 (M+1); MS (ES−): 493.20 (M−1).

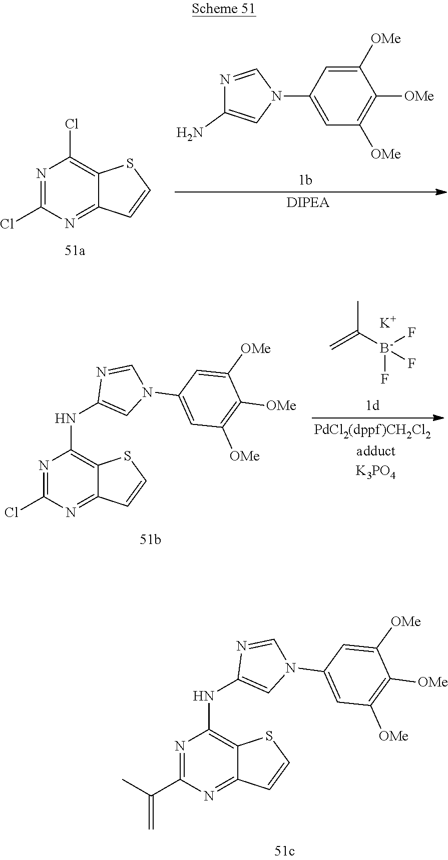

### Preparation of 2-(prop-1-en-2-yl)-N-(1-(3,4,5-trimethoxyphenyl)-1H-imidazol-4-yl)thieno[3,2-d]pyrimidin-4-amine (51c)

### Step-1: Preparation of 2-chloro-N-(1-(3,4,5-trimethoxyphenyl)-1H-imidazol-4-yl)thieno[3,2-d]pyrimidin-4-amine (51b)

Compound 51b was prepared according to the procedure reported in step-1 of scheme 1, from 2,4-dichlorothieno[3,2-d]pyrimidine (51a) (1.0 g, 4.88 mmol; CAS #16234-14-3) in 2-propanol (20 mL) using DIPEA (2.5 mL, 14.64 mmol), 1-(3,4,5-trimethoxyphenyl)-1H-imidazol-4-amine (1b) (1.22 g, 4.89 mmol) and heating at 80° C. for 2 h. This gave after work up 2-chloro-N-(1-(3,4,5-trimethoxyphenyl)-1H-imidazol-4-yl)thieno[3,2-d]pyrimidin-4-amine (51b) (0.86 g, 41% yield); ¹H NMR (300 MHz, DMSO-d₆) δ 10.82 (s, 1H), 8.24 (d, J=6.1 Hz, 2H), 7.95 (s, 1H), 7.39 (d, J=5.4 Hz, 1H), 6.96 (s, 2H), 3.88 (s, 6H), 3.70 (s, 3H).

### Step-2: Preparation of 2-(prop-1-en-2-yl)-N-(1-(3,4,5-trimethoxyphenyl)-1H-imidazol-4-yl)thieno[3,2-d]pyrimidin-4-amine (51c)

Compound 51c was prepared according to the procedure reported in step-1 of scheme 1, from 2-chloro-N-(1-(3,4,5-trimethoxyphenyl)-1H-imidazol-4-yl)thieno[3,2-d]pyrimidin-4-amine (51b) (2.0 g, 4.79 mmol) in toluene (100 mL) using potassium isopropenyltrifluoroborate (1d) (0.92 g, 6.22 mmol), and a solution of potassium phosphate (1.53 g, 7.2 mmol) in water (5.0 mL), PdCl₂(dppf)-CH₂Cl₂ adduct (392 mg, 0.48 mmol) and heating at 80° C. for 6 h. This gave after work up and purification by column chromatography [silica gel, eluting with DMA-80 in DCM from 5-10%] followed by triturating with IPA (30 mL), filtration and drying afforded 2-(prop-1-en-2-yl)-N-(1-(3,4,5-trimethoxyphenyl)-1H-imidazol-4-yl)thieno[3,2-d]pyrimidin-4-amine (51c) (1.23 g, 60% yield) as an off white solid; ¹H NMR (300 MHz, DMSO-d₆) δ 10.53 (s, 1H, D₂O exchangeable), 8.26 (d, J=1.5 Hz, 1H), 8.18 (d, J=5.4 Hz, 1H), 8.13 (d, J=1.6 Hz, 1H), 7.46 (d, J=5.4 Hz, 1H), 6.96 (s, 2H), 6.43 (d, J=2.8 Hz, 1H), 5.52 (d, J=2.5 Hz, 1H), 3.88 (s, 6H), 3.70 (s, 3H), 2.31 (s, 3H); MS (ES+): 424.10 (M+1); (ES−): 422.10 (M−1); Analysis calculated for C₂₁H₂₁N₅O₃S·0.25H₂O: C, 58.93; H, 5.06; N, 16.36; Found: C, 59.13; H, 4.99; N, 16.34.

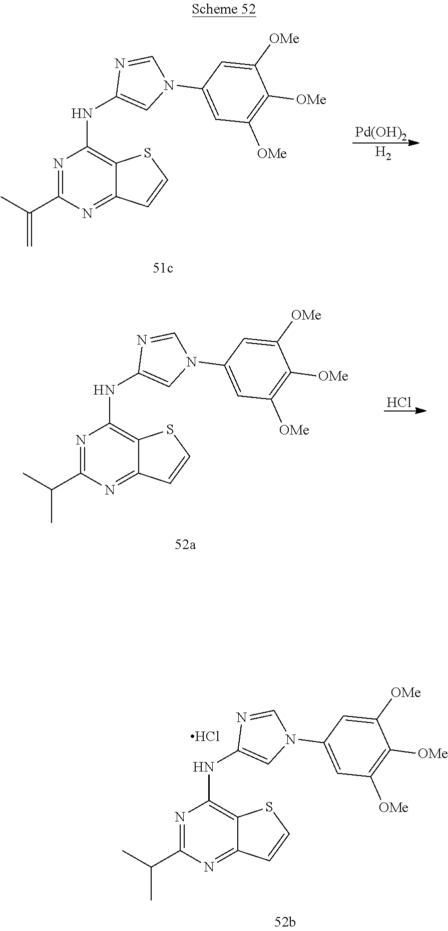

### Preparation of 2-isopropyl-N-(1-(3,4,5-trimethoxyphenyl)-1H-imidazol-4-yl)thieno[3,2-d]pyrimidin-4-amine hydrochloride (52b)

### Step-1: Preparation of 2-isopropyl-N-(1-(3,4,5-trimethoxyphenyl)-1H-imidazol-4-yl)thieno[3,2-d]pyrimidin-4-amine (52a)

Compound 52a was prepared according to the procedure reported in step-3 of scheme 1, from 2-(prop-1-en-2-yl)-N-(1-(3,4,5-trimethoxyphenyl)-1H-imidazol-4-yl)thieno[3,2-d]pyrimidin-4-amine (51c) (500 mg, 1.18 mmol) in MeOH:DCM (110 mL) using 50% wet 20% palladium hydroxide on carbon (164 mg, 0.12 mmol) and stirring at RT for 15 h under a H₂ atmosphere. This gave after work up and purification using column chromatography [silica gel, eluting with 10% DMA-80 in DCM] 2-isopropyl-N-(1-(3,4,5-trimethoxyphenyl)-1H-imidazol-4-yl)thieno[3,2-d]pyrimidin-4-amine (52a) (400 mg, 80% yield) as an off-white solid; ¹H NMR (300 MHz, DMSO-d₆) δ 10.39 (s, 1H), 8.22 (s, 1H), 8.18-8.08 (m, 2H), 7.38 (d, J=5.4 Hz, 1H), 6.94 (s, 2H), 3.88 (s, 6H), 3.70 (s, 3H), 3.20-3.00 (m, 1H), 1.37 (d, J=6.9 Hz, 6H).

### Step-2: Preparation of 2-isopropyl-N-(1-(3,4,5-trimethoxyphenyl)-1H-imidazol-4-yl)thieno[3,2-d]pyrimidin-4-amine hydrochloride (52b)

To a stirred solution of 2-isopropyl-N-(1-(3,4,5-trimethoxyphenyl)-1H-imidazol-4-yl)thieno[3,2-d]pyrimidin-4-amine (52a) (350 mg, 0.82 mmol) in ethanol (5 mL) was added 19% HCl in EtOH (2 mL) and stirred at RT for 1 h. The resulted precipitate was filtered, washed with MTBE (5 mL) and dried in an oven at 50° C. to afford 2-isopropyl-N-(1-(3,4,5-trimethoxyphenyl)-1H-imidazol-4-yl)thieno[3,2-d]pyrimidin-4-amine hydrochloride (52b) (300 mg, 79% yield) HCl salt as a white solid; ¹H NMR (300 MHz, DMSO-d₆) δ 8.46 (d, 1=30.7 Hz, 2H), 8.18 (d, J=1.6 Hz, 1H), 7.57 (d, J=5.4 Hz, 1H), 6.99 (s, 2H), 3.88 (s, 6H), 3.71 (s, 3H), 3.48-3.18 (m, 1H), 1.45 (d, J=6.8 Hz, 6H); MS (ES+): 426.20 (M+1); (ES−): 424.20 (M−1); Analysis calculated for C₂₁H₂₃N₅O₃S·1.5HCl·2.25H₂O: C, 48.44; H, 5.61; N, 13.45; Found: C, 48.28; H, 5.41; N, 13.30.

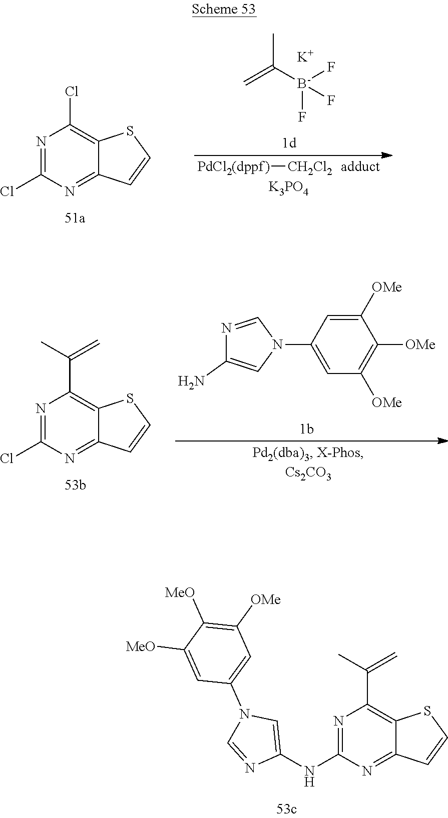

### Preparation of 4-(prop-1-en-2-yl)-N-(1-(3,4,5-trimethoxyphenyl)-1H-imidazol-4-yl)thieno[3,2-d]pyrimidin-2-amine (53c)

### Step-1: Preparation of 2-chloro-4-(prop-1-en-2-yl)thieno[3,2-d]pyrimidine (53b)

Compound 53b was prepared according to the procedure reported in step-1 of scheme 1, from 2,4-dichlorothieno[3,2-d]pyrimidine (51a) (3.0 g 14.63 mmol; CAS #16234-14-3) in toluene (60 mL) using potassium isopropenyltrifluoroborate (1d) a solution of potassium phosphate (4.66 g, 21.94 mmol) in water (3.0 mL), PdCl₂(dppf)-CH₂Cl₂ adduct (1.19 g, 1.46 mmol) and heating at 60° C. This gave after work up and purification by column chromatography [silica gel, eluting with EtOAc in n-heptane from 0% to 10%] 2-chloro-4-(prop-1-en-2-yl)thieno[3,2-d]pyrimidine (53b) (2.1 g, 68% yield) as an off white solid; ¹H NMR (300 MHz, DMSO-d₆) δ 8.63 (d, J=5.5 Hz, 1H), 7.67 (d, J=5.5 Hz, 1H), 6.26-6.08 (m, 1H), 6.03-5.88 (m, 1H), 2.26 (t, J=1.1 Hz, 3H).

### Step-2: Preparation of 4-(prop-1-en-2-yl)-N-(1-(3,4,5-trimethoxyphenyl)-1H-imidazol-4-yl)thieno[3,2-d]pyrimidin-2-amine (53c)

Compound 53c was prepared according to the procedure reported in step-4 of scheme 7, from 2-chloro-4-(prop-1-en-2-yl)thieno[3,2-d]pyrimidine (53b) (2.0 g, 9.49 mmol) in 1,4-dioxane (60 mL) using 1-(3,4,5-trimethoxyphenyl)-1H-imidazol-4-amine (1b) (3.1 g, 12.34 mmol) cesium carbonate (9.27 g, 28.5 mmol), Pd₂(dba)₃ (870 mg, 0.95 mmol), XPhos (1.8 g, 3.8 mmol) and heating at 100° C. for 6 h under argon. This gave after workup and purification using flash column chromatography [silica gel (24 g), eluting with 10% DMA-80 in DCM] 4-(prop-1-en-2-yl)-N-(1-(3,4,5-trimethoxyphenyl)-1H-imidazol-4-yl)thieno[3,2-d]pyrimidin-2-amine (53c) (1.70 g, 42% yield) as a fluorescent yellow solid; ¹H NMR (300 MHz, DMSO-d₆) δ 9.76 (s, 1H D₂O exchangeable), 8.32 (d, J=5.5 Hz, 1H), 8.12 (d, J=1.6 Hz, 1H), 7.90 (s, 1H), 7.45 (d, J=5.6 Hz, 1H), 6.93 (s, 2H), 6.07 (s, 1H), 5.87 (s, 1H), 3.89 (s, 6H), 3.69 (s, 3H), 2.34 (s, 3H); MS (ES+): 424.10 (M+1); Analysis calculated for C₂₁H₂₁N₅O₃S: C, 59.56; H, 5.00; N, 16.54; Found: C, 59.28; H, 4.92; N, 16.52.

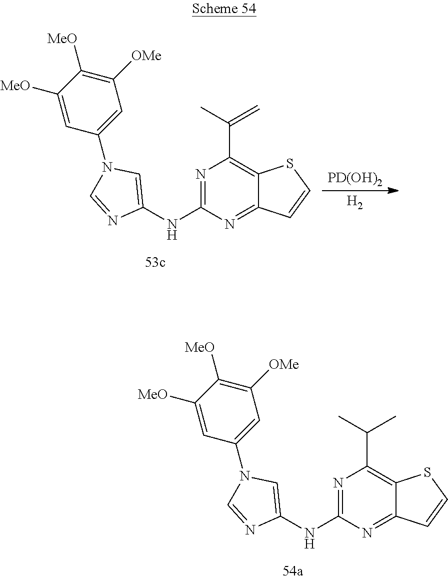

### Preparation of 4-isopropyl-N-(1-(3,4,5-trimethoxyphenyl)-1H-imidazol-4-yl)thieno[3,2-d]pyrimidin-2-amine (54a)

Compound 54a was prepared according to the procedure reported in step-3 of scheme 1, from 4-(prop-1-en-2-yl)-N-(1-(3,4,5-trimethoxyphenyl)-1H-imidazol-4-yl)thieno[3,2-d]pyrimidin-2-amine (53c) (500 mg, 1.18 mmol) in MeOH:DCM (ratio: 10:1, 110 mL) using 50% wet, 20% palladium hydroxide on carbon (168 mg, 0.24 mmol) and stirring for 15 h at RT under a H₂ atmosphere. This gave after work up and purification using column chromatography [silica gel, eluting with 10% DMA-80 in DCM] 4-isopropyl-N-(1-(3,4,5-trimethoxyphenyl)-1H-imidazol-4-yl)thieno[3,2-d]pyrimidin-2-amine (54a) (350 mg, 70% yield) as white solid; ¹H NMR (300 MHz, DMSO-d₆) δ 9.65 (s, 1H, D₂O exchangeable), 8.26 (d, J=5.4 Hz, 1H), 8.10 (d, J=1.7 Hz, 1H), 7.92 (d, J=1.6 Hz, 1H), 7.41 (d, J=5.4 Hz, 1H), 6.92 (s, 2H), 3.89 (s, 6H), 3.69 (s, 3H), 3.30-3.16 (m, 1H), 1.41 (d, J=6.8 Hz, 6H); MS (ES+): 426.15 (M+1); Analysis calculated for C₂₁H₂₃N₅O₃S: C, 59.28; H, 5.45; N, 16.46; Found: C, 59.24; H, 5.48; N, 16.44.

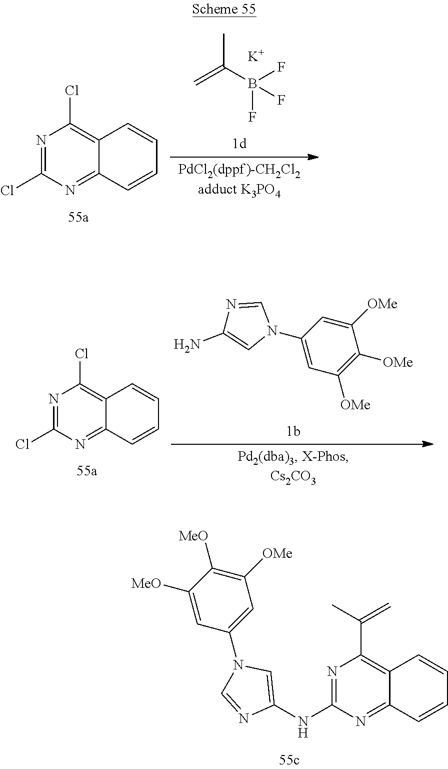

### Preparation of 4-(prop-1-en-2-yl)-N-(1-(3,4,5-trimethoxyphenyl)-1H-imidazol-4-yl)quinazolin-2-amine (55c)

### Step-1: Preparation of 2-chloro-4-(prop-1-en-2-yl)quinazoline (55b)

Compound 55b was prepared according to the procedure reported in step-1 of scheme 1, from 2,4-dichloroquinazoline (55a) (3.0 g, 15.073 mmol; CAS #607-68-1) in toluene (49.8 mL) using potassium isopropenyltrifluoroborate (1d) (2.23 g, 15.073 mmol), potassium phosphate (4.799 g, 22.609 mmol), PdCl₂(dppf)-CH₂Cl₂ adduct (1.846 g, 2.261 mmol) and heating at reflux for 1 h. This gave after work up and purification using column chromatography [silica gel, eluting with EtOAc in n-heptane from 0-8%)] 2-chloro-4-(prop-1-en-2-yl)quinazoline (55b) (2 g, 65% yield) as a light yellow solid; ¹H NMR (300 MHz, DMSO-d₆) δ 8.27 (dt, J=8.6, 1.8 Hz, 1H), 8.13-8.04 (m, 1H), 8.01-7.94 (m, 1H), 7.86-7.64 (m, 1H), 5.87 (s, 1H), 5.46 (s, 1H), 2.24 (s, 3H).

### Step-2: Preparation of 4-(prop-1-en-2-yl)-N-(1-(3,4,5-trimethoxyphenyl)-1H-imidazol-4-yl)quinazolin-2-amine (55c)

Compound 55c was prepared according to the procedure reported in step-4 of scheme 7, from 2-chloro-4-(prop-1-en-2-yl)quinazoline (55b) (1 g, 4.89 mmol) in 1,4-dioxane (30 mL) using 1-(3,4,5-trimethoxyphenyl)-1H-imidazol-4-amine (1b) (1.22 g, 4.89 mmol), cesium carbonate (4.77 g, 14.66 mmol), Pd₂(dba)₃ (0.671 g, 0.73 mmol), XPhos (0.931 g, 1.95 mmol) and heating at 100° C. for 12 h under argon. This gave after workup and purification using flash column chromatography [silica gel (24 g), eluting with EtOAc in n-heptane from 0-85%] 4-(prop-1-en-2-yl)-N-(1-(3,4,5-trimethoxyphenyl)-1H-imidazol-4-yl)quinazolin-2-amine (55c) (0.280 g, 14% yield) as a yellow solid; ¹H NMR (300 MHz, DMSO-d₆) δ 9.96 (s, 1H, D₂O exchangeable), 8.11 (d, J=1.6 Hz, 1H), 8.09-7.98 (m, 2H), 7.80 (d, J=7.9 Hz, 2H), 7.41-7.24 (m, 11H), 6.95 (s, 2H), 5.75 (s, 1H), 5.36 (s, 1H), 3.91 (s, 6H), 3.70 (s, 3H), 2.26 (s, 3H); MS (ES+): 418.20 (M+1).

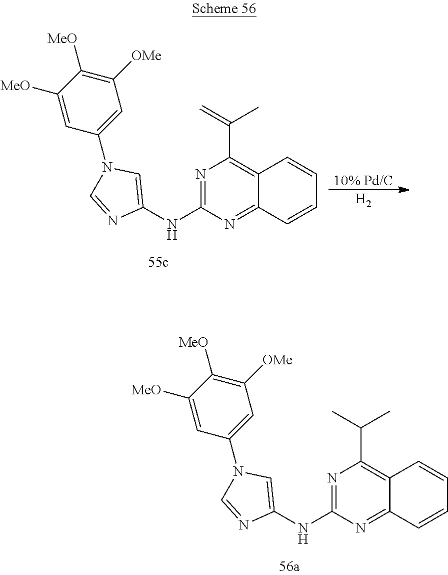

### Preparation of 4-isopropyl-N-(1-(3,4,5-trimethoxyphenyl)-1H-imidazol-4-yl)quinazolin-2-amine (56a)

Compound 56a was prepared according to the procedure reported in scheme 41, from 4-(prop-1-en-2-yl)-N-(1-(3,4,5-trimethoxyphenyl)-1H-imidazol-4-yl)quinazolin-2-amine (55c) (0.18 g, 0.43 mmol) in ethanol (7.2 mL) and acetic acid (7.2 mL) using 50% wet, 10% Pd/C (0.183 g, 0.086 mmol) and stirring at RT for 12 h under a H₂ atmosphere. This gave after work up and purification using column chromatography [silica gel, eluting with MeOH in DCM from 0-4%] 4-isopropyl-N-(1-(3,4,5-trimethoxyphenyl)-1H-imidazol-4-yl)quinazolin-2-amine (56a) (0.045 g, 25% yield) as a brown solid; ¹H NMR (300 MHz, DMSO-d₆) δ 9.75 (s, 1H, D₂O exchangeable), 8.13 (d, J=9.7 Hz, 2H), 8.06 (s, 1H), 7.76 (s, 2H), 7.35 (d, J=8.9 Hz, 1H), 6.94 (d, J=2.0 Hz, 2H), 4.08-3.81 (m, 7H), 3.70 (s, 3H), 1.38 (d, J=6.6 Hz, 6H); MS (ES+): 420.2 (M+1).

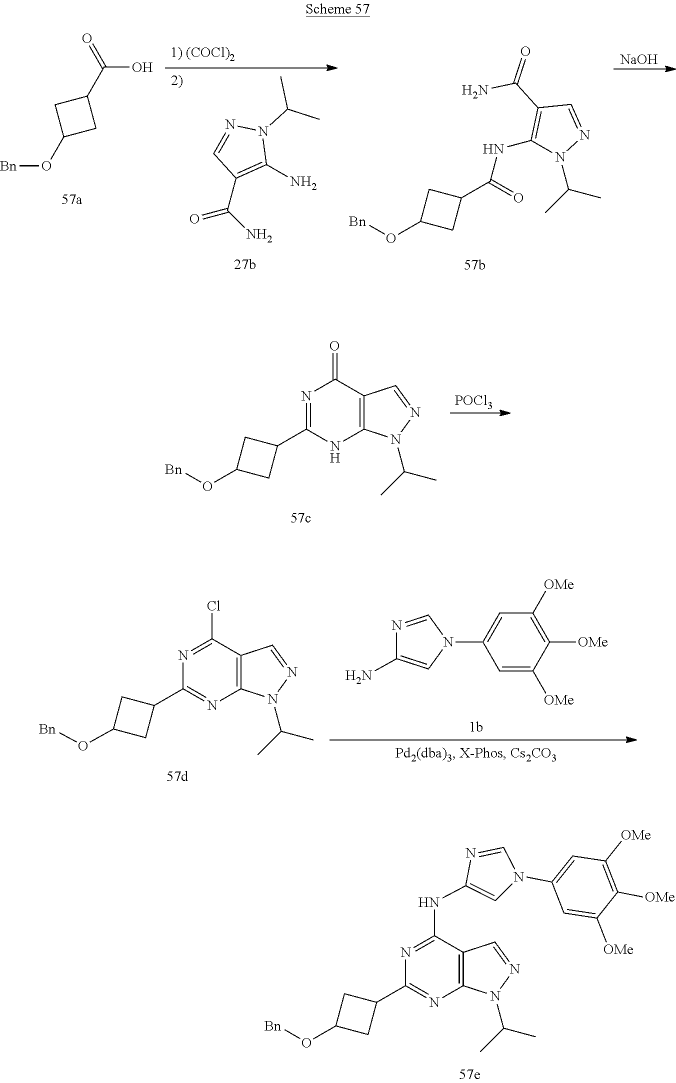

### Preparation of 6-(3-(benzyloxy)cyclobutyl)-1-isopropyl-N-(1-(3,4,5-trimethoxyphenyl)-1H-imidazol-4-yl)-1H-pyrazolo[3,4-d]pyrimidin-4-amine (57e)

### Step-1: Preparation of 5-(3-(benzyloxy)cyclobutanecarboxamido)-1-isopropyl-1H-pyrazole-4-carboxamide (57b)

Compound 57b was prepared according to the procedure reported in step-1 of scheme 27, from 3-(benzyloxy)cyclobutanecarboxylic acid (57a) (4.0 g, 19.39 mmol; CAS #4958-02-5) in DCM (40.0 mL) using oxalyl chloride (7.38 g, 58.14 mmol), 5-amino-1-isopropyl-1H-pyrazole-4-carboxamide (27b) (2.3 g, 13.67 mmol) in 1,4-dioxane (23 mL) and stirring at RT for 12 h. This gave after work up and purification using column chromatography [silica gel, eluting with 5% MeOH in DCM] 5-(3-(benzyloxy)cyclobutanecarboxamido)-1-isopropyl-1H-pyrazole-4-carboxamide (57b) (2.5 g, 52% yield) as an off-white solid; ¹H NMR (300 MHz, DMSO-d₆) δ 9.68 (d, J=6.2 Hz, 1H), 7.87 (s, 1H), 7.43-7.20 (m, 5H), 6.96 (s, 1H), 4.38 (s, 2H), 4.34-4.14 (m, 1H), 4.05-3.92 (m, 1H), 2.95-2.69 (m, 1H), 2.50-2.38 (m, 2H), 2.36-1.95 (m, 2H), 1.31 (d, J=6.6 Hz, 6H).

### Step-2: Preparation of 6-(3-(benzyloxy)cyclobutyl)-1-isopropyl-1H-pyrazolo[3,4-d]pyrimidin-4 (7H)-one (57c)

Compound 57c was prepared according to the procedure reported in step-2 of scheme 27, from 5-(3-(benzyloxy)cyclobutanecarboxamido)-1-isopropyl-1H-pyrazole-4-carboxamide (57b) (2.5 g, 7.01 mmol) using a solution of NaOH (2N) (2.80 g, 70.00 mmol) and heating at 70° C. for 0.5 h. This gave after work up 6-(3-(benzyloxy)cyclobutyl)-1-isopropyl-1H-pyrazolo[3,4-d]pyrimidin-4 (7H)-one (57c) (2.0 g, 84% yield) as an off white solid; ¹H NMR (300 MHz, DMSO-d₆) δ 11.97 (s, 1H), 8.01-7.95 (m, 1H), 7.42-7.26 (m, 5H), 5.06-4.89 (m, 1H), 4.45-4.38 (m, 2H), 4.11-3.95 (m, 1H), 3.12-2.95 (m, 1H), 2.66-2.52 (m, 2H), 2.40-2.18 (m, 2H), 1.45 (d, J=6.7 Hz, 6H).

### Step-3: Preparation of 6-(3-(benzyloxy)cyclobutyl)-4-chloro-1-isopropyl-1H-pyrazolo[3,4-d]pyrimidine (57d)

Compound 57d was prepared according to the procedure reported in step-3 of scheme 27, from 6-(3-(benzyloxy)cyclobutyl)-1-isopropyl-1H-pyrazolo[3,4-d]pyrimidin-4 (7H)-one (57c) (2.5 g, 7.38 mmol) using POCl₃ (64.56 g, 421.08 mmol) and heating at 100° C. for 1 h. This gave after work up and purification using column chromatography [silica gel, eluting with 10% MeOH in DCM] 6-(3-(benzyloxy)cyclobutyl)-4-chloro-1-isopropyl-1H-pyrazolo[3,4-d]pyrimidine (57d) (1.9 g, 72% yield) as an oil; ¹H NMR (300 MHz, DMSO-d₆) δ 8.38 (s, 1H), 7.40-7.26 (m, 5H), 5.22-5.06 (m, 1H), 4.43 (d, J=3.8 Hz, 2H), 4.17-4.01 (m, 1H), 3.42-3.24 (m, 11H), 2.76-2.56 (m, 2H), 2.42-2.21 (m, 2H), 1.50 (d, J=6.7 Hz, 6H).

### Step-4: Preparation of 6-(3-(benzyloxy)cyclobutyl)-1-isopropyl-N-(1-(3,4,5-trimethoxyphenyl)-1H-imidazol-4-yl)-1H-pyrazolo[3,4-d]pyrimidin-4-amine (57e)

Compound 57e was prepared according to the procedure reported in step-4 of scheme 27, from 6-(3-(benzyloxy)cyclobutyl)-4-chloro-1-isopropyl-1H-pyrazolo[3,4-d]pyrimidine (57d) (1.0 g, 2.8 mmol) in 1,4-dioxane (18 mL) using 1-(3,4,5-trimethoxyphenyl)-1H-imidazol-4-amine (1b) (0.9 g, 3.64 mmol), Pd₂(dba)₃ (0.51 g, 0.56 mmol), X-phos (0.529 g 1.12 mmol), Cs₂CO3 (2.73 g, 8.4 mmol) and heating at 120° C. for 4 h. This gave after work up and purification using column chromatography [silica gel, eluting with 10% MeOH in DCM] followed by purification using reverse phase column chromatography [C18 column (130 g), eluting with ACN in water (containing 0.1% HCl) from 0-100%] 6-(3-(benzyloxy)cyclobutyl)-1-isopropyl-N-(1-(3,4,5-trimethoxyphenyl)-1H-imidazol-4-yl)-1H-pyrazolo[3,4-d]pyrimidin-4-amine (57e) (145 mg, 41%) HCl salt as a white solid; ¹H NMR (300 MHz, DMSO-d₆) δ 11.24 (s, 1H, D₂O exchangeable), 8.41 (d, J=13.9 Hz, 2H), 8.22-8.05 (m, 1H), 7.37-7.11 (m, 5H), 6.98 (d, J=3.3 Hz, 2H), 5.21-4.91 (m, 1H), 4.34 (d, J=12.4 Hz, 2H), 4.11-4.01 (m, 1H), 3.86 (d, J=2.4 Hz, 6H), 3.65 (s, 3H), 3.34-3.13 (m, 1H), 2.79-2.58 (m, 2H), 2.46-2.25 (m, 2H), 1.59-1.35 (m, 6H); MS (ES+): 570.2 (M+1); C₃₁H₃₅N₇O₄·1HCl·1.75H₂O: C, 58.39; H, 6.24; Cl, 5.56; N, 15.38; Found: C, 58.26; H, 6.30; Cl, 5.38; N, 15.29.

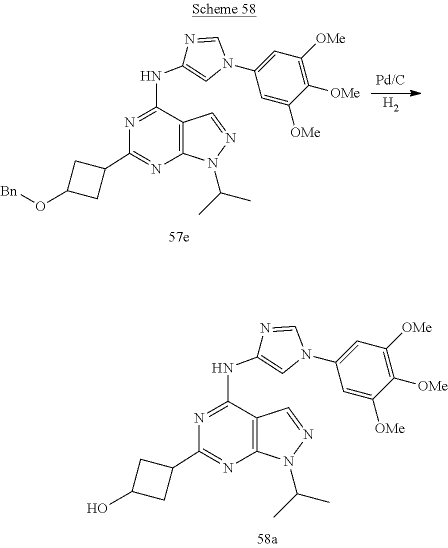 [SMILES: COc1cc(-n2cnc(Nc3nc(C4CC(OCc5ccccc5)C4)nc4c3cnn4C(C)C)c2)cc(OC)c1OC ; "H2" ; COc1cc(-n2cnc(Nc3nc(C4CC(O)C4)nc4c3cnn4C(C)C)c2)cc(OC)c1OC]

### Preparation of 3-(1-isopropyl-4-((1-(3,4,5-trimethoxyphenyl)-1H-imidazol-4-yl)amino)-1H-pyrazolo[3,4-d]pyrimidin-6-yl)cyclobutanol (58a)

Compound 58a was prepared according to the procedure reported in scheme 41, from 6-(3-(benzyloxy)cyclobutyl)-1-isopropyl-N-(1-(3,4,5-trimethoxyphenyl)-1H-imidazol-4-yl)-1H-pyrazolo[3,4-d]pyrimidin-4-amine (57e) (0.3 g, 0.526 mmol) in ethanol (10.0 mL) using ammonium formate (0.132 mg, 2.093 mmol), 10% Pd/C (0.022 g, 0.206 mmol) and heating at 80° C. for 1 h under a H₂ atmosphere. This gave after work up and purification by column chromatography [silica gel, eluting with 10% MeOH in DCM] followed by purification using reverse phase column chromatography [C18 column (130 g), eluting with ACN in water (containing 0.1% HCl) from 0-100%] 3-(1-isopropyl-4-((1-(3,4,5-trimethoxyphenyl)-1H-imidazol-4-yl)amino)-1H-pyrazolo[3,4-d]pyrimidin-6-yl)cyclobutanol (58a) (0.049 g, 49% yield) HCl salt as a white solid; ¹H NMR (300 MHz, DMSO-d₆) δ 11.52 (s, 1H, D₂O exchangeable), 8.63-8.28 (m, 2H), 8.12 (dd, J=25.5, 1.6 Hz, 1H), 6.98 (d, J=4.3 Hz, 2H), 5.15-4.98 (m, 1H), 4.20-4.07 (m, 1H), 3.88 (s, 6H), 3.69 (s, 3H), 3.27-3.04 (m, 1H), 2.76-2.55 (m, 2H), 2.43-2.22 (m, 2H), 1.45 (dd, J=6.7, 2.5 Hz, 6H); MS (ES+): 480.1 (M+1); Analysis calculated for C₂₄H₂₉N₇O₄·1HCl·1.75H₂O: C, 52.65; H, 6.17; Cl, 6.48; N, 17.91; Found: C, 52.87; H, 6.19; Cl, 6.33; N, 17.89.

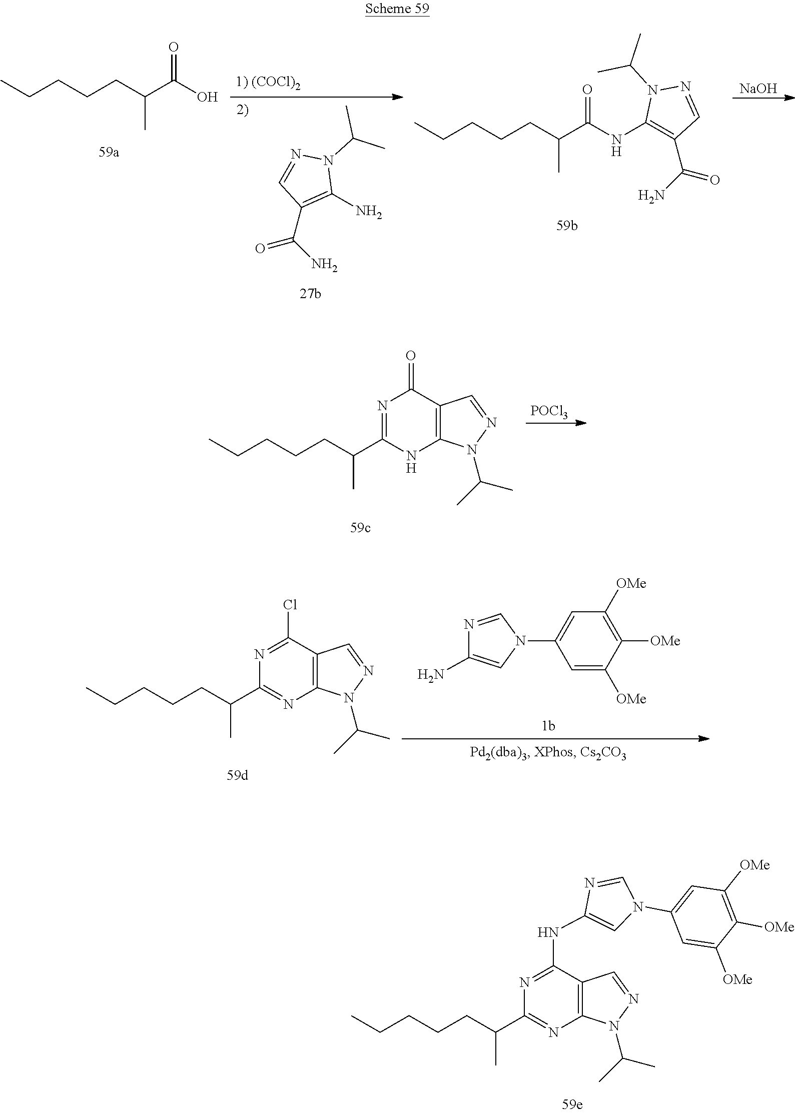

### Preparation of 6-(heptan-2-yl)-1-isopropyl-N-(1-(3,4,5-trimethoxyphenyl)-1H-imidazol-4-yl)-1H-pyrazolo[3,4-d]pyrimidin-4-amine (59e)

### Step-1: Preparation of 1-isopropyl-5-(2-methylheptanamido)-1H-pyrazole-4-carboxamide (59b)

Compound 59b was prepared according to the procedure reported in step-1 of scheme 27, from 2-methylheptanoic acid (59a) (1.0 g, 6.93 mmol; CAS #1188-02-9) in DCM (10 mL) using oxalyl chloride (2.64 g, 20.79 mmol), 2 drops of DMF, 5-amino-1-isopropyl-1H-pyrazole-4-carboxamide (27b) (0.776 g, 4.61 mmol) in 1,4-dioxane (7 mL) and stirring at RT for 12 h. This gave after work up and purification using column chromatography [silica gel, eluting with 5% MeOH in DCM] 1-isopropyl-5-(2-methylheptanamido)-1H-pyrazole-4-carboxamide (59b) (460 mg, 34% yield) as an off-white solid; ¹H NMR (300 MHz, DMSO-d₆) δ 9.70 (s, 1H), 7.87 (s, 1H), 7.23 (s, 1H), 6.98 (s, 1H), 4.37-4.16 (m, 1H), 1.72-1.50 (m, 1H), 1.39-1.16 (m, 14H), 1.10 (d, J=6.8 Hz, 3H), 0.91-0.78 (m, 3H).

### Step-2: Preparation of 6-(heptan-2-yl)-1-isopropyl-1H-pyrazolo[3,4-d]pyrimidin-4 (7H)-one (59c)

Compound 59c was prepared according to the procedure reported in step-2 of scheme 27, from 1-isopropyl-5-(2-methylheptanamido)-1H-pyrazole-4-carboxamide (59b) (1.0 g, 3.396 mmol) using a solution of NaOH (2N) (1.35 g, 33.75 mmol) and heating at 70° C. for 0.5 h. This gave after work up 6-(heptan-2-yl)-1-isopropyl-1H-pyrazolo[3,4-d]pyrimidin-4 (7H)-one (59c) (700 mg, 75% yield) as an off-white solid; ¹H NMR (300 MHz, DMSO-d₆) δ 11.95 (s, 1H), 7.98 (s, 1H), 5.04-4.78 (m, 1H), 2.88-2.71 (m, 1H), 1.87-1.65 (m, 1H), 1.59-1.38 (m, 7H), 1.35-1.12 (m, 9H), 0.96-0.69 (m, 3H).

### Step-3: Preparation of 4-chloro-6-(heptan-2-yl)-1-isopropyl-1H-pyrazolo[3,4-d]pyrimidine (59d)

Compound 59d was prepared according to the procedure reported in step-3 of scheme 27, from 6-(heptan-2-yl)-1-isopropyl-1H-pyrazolo[3,4-d]pyrimidin-4 (7H)-one (59c) (0.7 g, 2.53 mmol) using POCl₃ (22.52 g, 146.899 mmol) and heating at 100° C. for 1 h. This gave after work up and purification using column chromatography [silica gel, eluting with 30% EtOAc in n-heptane] 4-chloro-6-(heptan-2-yl)-1-isopropyl-1H-pyrazolo[3,4-d]pyrimidine (59d) (0.5 g, 67% yield) as an oily mass; ¹H NMR (300 MHz, DMSO-d₆) δ 11.96 (s, 1H), 7.98 (s, 1H), 5.00-4.83 (m, 1H), 2.89-2.71 (m, 1H), 1.82-1.69 (m, 1H), 1.54-1.48 (m, 1H), 1.44 (dd. J=6.7, 3.3 Hz, 6H), 1.33-1.16 (m, 9H), 0.91-0.77 (m, 3H).

### Step-4: Preparation of 6-(heptan-2-yl)-1-isopropyl-N-(1-(3,4,5-trimethoxyphenyl)-1H-imidazol-4-yl)-1H-pyrazolo[3,4-d]pyrimidin-4-amine (59e)

Compound 59e was prepared according to the procedure reported in step-4 of scheme 27, from 4-chloro-6-(heptan-2-yl)-1-isopropyl-1H-pyrazolo[3,4-d]pyrimidine (59d) (500 mg, 1.695 mmol) in 1,4-dioxane (15.0 mL) using 1-(3,4,5-trimethoxyphenyl)-1H-imidazol-4-amine (1b) (0.549 g, 2.202 mmol), Pd₂(dba)₃ (0.310 g, 0.338 mmol), X-phos (0.323 g 0.667 mmol). Cs₂CO₃ (1.65 g, 5.12 mmol) and heating at 120° C. for 12 h. This gave after work up and purification using column chromatography [silica gel, eluting with 10% MeOH in DCM] followed by purification using reverse phase column chromatography [C18 column (130 g), eluting with ACN in water (containing 0.1% HCl) from 0-100%] 6-(heptan-2-yl)-1-isopropyl-N-(1-(3,4,5-trimethoxyphenyl)-1H-imidazol-4-yl)-1H-pyrazolo[3,4-d]pyrimidin-4-amine (59e) (0.048 g, 48% yield) HCl salt as a white solid; ¹H NMR (300 MHz, DMSO-d₆) δ 11.40 (s, 1H, D₂O exchangeable), 8.41 (s, 2H), 8.08 (d, J=1.6 Hz, 1H), 6.96 (s, 2H), 5.21-4.94 (m, 1H), 3.88 (s, 6H), 3.70 (s, 3H), 3.12-2.83 (m, 1H), 2.01-1.77 (m, 1H), 1.72-1.55 (m, 1H), 1.46 (d, J=6.6 Hz, 6H), 1.34 (d, J=6.8 Hz, 3H), 1.30-1.14 (m, 6H), 0.85-0.60 (m, 3H); MS (ES+): 508.2 (M+1); Analysis calculated for C₂₇H₃₇N₇O₃·HCl·2H₂O: C, 55.90; H, 7.30; Cl, 6.11; N, 16.90; Found: C, 56.59; H, 7.32; Cl, 5.74; N, 16.66.

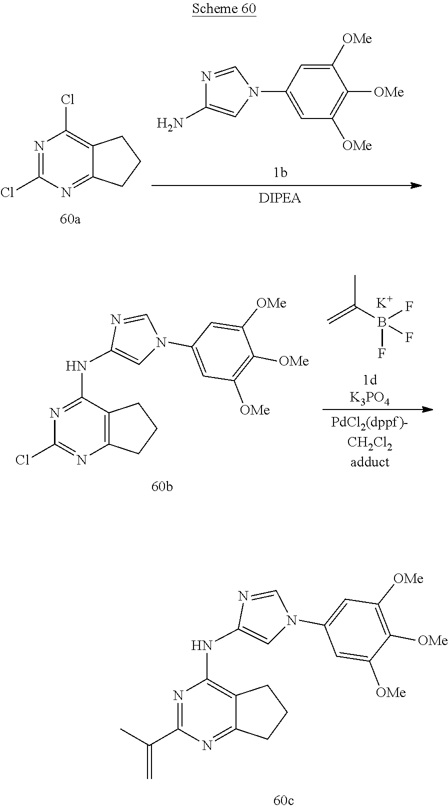

### Preparation of 2-(prop-1-en-2-yl)-N-(1-(3,4,5-trimethoxyphenyl)-1H-imidazol-4-yl)-6,7-dihydro-5H-cyclopenta[d]pyrimidin-4-amine (60c)

### Step-1: Preparation of 2-chloro-N-(1-(3,4,5-trimethoxyphenyl)-1H-imidazol-4-yl)-6,7-dihydro-5H-cyclopenta[d]pyrimidin-4-amine (60b)

Compound 60b was prepared according to the procedure reported in step-1 of scheme 1, from 2,4-dichloro-6,7-dihydro-5H-cyclopenta[d]pyrimidine (60a) (0.5 g, 2.64 mmol; CAS #5466-43-3) in EtOH (10.0 mL) and DCM (2.0 mL) using DIPEA (1.0 g, 7.97 mmol), 1-(3,4,5-trimethoxyphenyl)-1H-imidazol-4-amine (1b) (0.79 g, 3.17 mmol) and heating at reflux for 12 h. This gave after work up 2-chloro-N-(1-(3,4,5-trimethoxyphenyl)-1H-imidazol-4-yl)-6,7-dihydro-5H-cyclopenta[d]pyrimidin-4-amine (60b) (0.28 g, 26% yield) as a brown solid; ¹H NMR (300 MHz, DMSO-d₆) δ 9.89 (s, 1H), 8.14 (d, J=1.6 Hz, 1H), 7.76 (d, J=1.6 Hz, 1H), 6.90 (s, 2H), 3.87 (s, 6H), 3.69 (s, 3H), 2.85-2.74 (m, 4H), 2.12-1.96 (m, 2H).

### Step-2: Preparation of 2-(prop-1-en-2-yl)-N-(1-(3,4,5-trimethoxyphenyl)-1H-imidazol-4-yl)-6,7-dihydro-5H-cyclopenta[d]pyrimidin-4-amine (60c)

Compound 60c was prepared according to the procedure reported in step-1 of scheme 1, from 2-chloro-N-(1-(3,4,5-trimethoxyphenyl)-1H-imidazol-4-yl)-6,7-dihydro-5H-cyclopenta[d]pyrimidin-4-amine (60b) (0.28 g, 0.70 mmol) in 1,4-dioxane (8.4 mL) using potassium isopropenyltrifluoroborate (I d) (0.15 g, 1.39 mmol), a solution of potassium phosphate (0.44 g, 2.08 mmol) in water (0.84 mL). PdCl₂(dppf)-CH₂Cl₂ adduct (0.11 g, 0.139 mmol) and heating at 100° C. for 12 h. This gave after work up and purification using column chromatography [silica gel (24 g), eluting with MeOH in DCM from 0% to 5%] 2-(prop-1-en-2-yl)-N-(1-(3,4,5-trimethoxyphenyl)-1H-imidazol-4-yl)-6,7-dihydro-5H-cyclopenta[d]pyrimidin-4-amine (60c) (0.25 g, 88% yield) as an off-white solid; ¹H NMR (300 MHz, DMSO-d₆) δ 9.49 (s, 1H, D₂O exchangeable), 8.20 (d, J=1.5 Hz, 1H), 8.03 (d, J=1.6 Hz, 1H), 6.92 (s, 2H), 6.33 (d, J=2.8 Hz, 1H), 5.43 (s, 1H), 3.87 (s, 6H), 3.69 (s, 3H), 2.96-2.76 (m, 4H), 2.25 (s, 3H), 2.13-1.93 (m, 2H); MS (ES+): 408.30 (M+1).

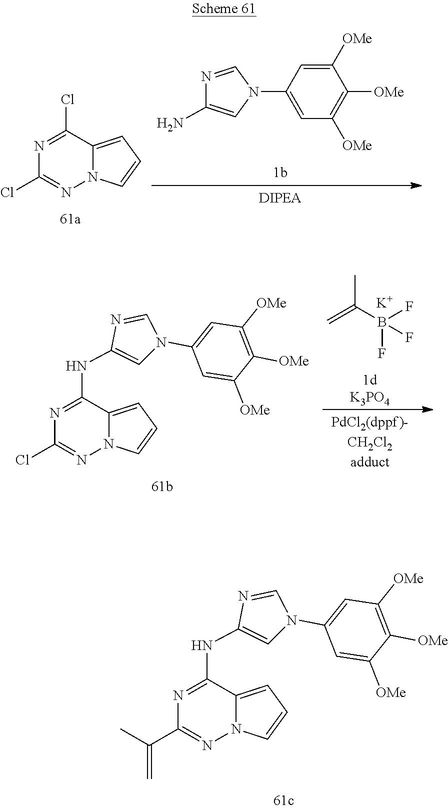

### Preparation of 2-(prop-1-en-2-yl)-N-(1-(3,4,5-trimethoxyphenyl)-1H-imidazol-4-yl)pyrrolo[2,1-f][1,2,4]triazin-4-amine (61c)

### Step-1: Preparation of 2-chloro-N-(1-(3,4,5-trimethoxyphenyl)-1H-imidazol-4-yl)pyrrolo[2,1-f][1,2,4]triazin-4-amine (61b)

Compound 61b was prepared according to the procedure reported in step-1 of scheme 1, from 2,4-dichloropyrrolo[2,1-f][1,2,4]triazine (61a) (0.5 g, 2.66 mmol; CAS #918538-05-3) in EtOH (10.0 mL) and DCM (2.0 mL) using DIPEA (1.0 g, 7.97 mmol), 1-(3,4,5-trimethoxyphenyl)-1H-imidazol-4-amine (1b) (0.795 g, 3.19 mmol) and stirring at RT for 12 h. This gave after work up 2-chloro-N-(1-(3,4,5-trimethoxyphenyl)-1H-imidazol-4-yl)pyrrolo[2,1-f][1,2,4]triazin-4-amine (61b) (0.65 g, 60% yield) as a brown solid H NMR (300 MHz, DMSO-d₆) δ 11.31 (s, 1H), 8.21 (d, J=1.6 Hz, 1H), 7.89 (d, J=1.6 Hz, 1H), 7.77 (dd, J=2.6, 1.5 Hz, 11H), 7.39 (d, J=4.5 Hz, 1H), 6.94 (s, 2H), 6.72 (dd, J=4.5, 2.6 Hz, 1H), 3.88 (s, 6H), 3.70 (s, 3H).

### Step-2: Preparation of 2-(prop-1-en-2-yl)-N-(1-(3,4,5-trimethoxyphenyl)-1H-imidazol-4-yl)pyrrolo[2,1-f][1,2,4]triazin-4-amine (61c)

Compound 61c was prepared according to the procedure reported in step-1 of scheme 1, from 2-chloro-N-(1-(3,4,5-trimethoxyphenyl)-1H-imidazol-4-yl)pyrrolo[2,1-f][1,2,4]triazin-4-amine (61b) (0.5 g, 1.25 mmol) in 1,4-dioxane (15 mL) using potassium isopropenyltrifluoroborate (Id)(0.369 g, 2.49 mmol), a solution of potassium carbonate (0.517 g, 3.74 mmol) in water (3 mL), PdCl₂(dppf)-CH₂Cl₂ adduct (0.203 g, 0.249 mmol) and heating at 100° C. for 12 h. This gave after work up and purification using column chromatography [silica gel, eluting with MeOH in DCM from 0% to 5%] 2-(prop-1-en-2-yl)-N-(1-(3,4,5-trimethoxyphenyl)-1H-imidazol-4-yl)pyrrolo[2,1-f][1,2,4]triazin-4-amine (61c) (0.3 g, 59% yield) as an off-white solid; ¹H NMR (300 MHz, DMSO-d₆) δ 10.80 (s, 1H, D₂O exchangeable), 8.25 (d, J=1.6 Hz, 1H), 8.07 (d, J=1.6 Hz, 1H), 7.75 (dd, J=2.6, 1.5 Hz, 1H), 7.32 (d, J=4.4 Hz, 1H), 6.95 (s, 2H), 6.70 (dd, J=4.3, 2.6 Hz, 1H), 6.34 (d, 1H), 5.52 (s, 1H), 3.88 (s, 6H), 3.70 (s, 3H), 2.18 (s, 3H); MS (ES+): 407.20 (M+1); (ES−): 405.20 (M−1).

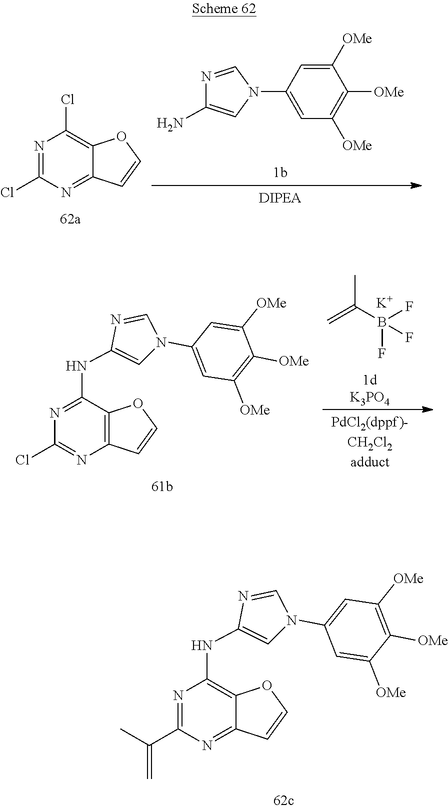

### Preparation of 2-(prop-1-en-2-yl)-N-(1-(3,4,5-trimethoxyphenyl)-1H-imidazol-4-yl)furo[3,2-d]pyrimidin-4-amine (62c)

### Step-1: Preparation of 2-chloro-N-(1-(3,4,5-trimethoxyphenyl)-1H-imidazol-4-yl)furo[3,2-d]pyrimidin-4-amine (62b)

Compound 62b was prepared according to the procedure reported in step-1 of scheme 1, from 2,4-dichlorofuro[3,2-d]pyrimidine (62a) (1.0 g, 5.29 mmol; CAS #956034-07-4) in EtOH (20 mL) using DIPEA (2.05 g, 15.86 mmol) and 1-(3,4,5-trimethoxyphenyl)-1H-imidazol-4-amine (1b) (1.45 g, 5.82 mmol) and stirring at RT for 12 h. This gave after work up 2-chloro-N-(1-(3,4,5-trimethoxyphenyl)-1H-imidazol-4-yl)furo[3,2-d]pyrimidin-4-amine (62b) (1.0 g, 47% yield) as a brown solid; ¹H NMR (300 MHz, DMSO-d₆) δ 10.88 (s, 1H), 8.36 (di. J=2.2 Hz, 1H), 8.17 (d, J=1.6 Hz, 1H), 7.85 (d, J=1.6 Hz, 1H), 7.04 (d, J=2.2 Hz, 1H), 6.93 (s, 2H), 3.87 (s, 6H), 3.69 (s, 3H).

### Step-2: Preparation of 2-(prop-1-en-2-yl)-N-(1-(3,4,5-trimethoxyphenyl)-1H-imidazol-4-yl)furo[3,2-d]pyrimidin-4-amine (62c)

Compound 62c was prepared according to the procedure reported in step-1 of scheme 1, from 2-chloro-N-(1-(3,4,5-trimethoxyphenyl)-1H-imidazol-4-yl)furo[3,2-d]pyrimidin-4-amine (62b) (1.5 g, 3.73 mmol) in 1,4-dioxane (30 mL) using potassium isopropenyltrifluoroborate (1d) (0.828 g, 5.59 mmol), a solution of potassium phosphate (1.18 g, 5.59 mmol) in water (2.0 mL), PdCl₂(dppf)-CH₂Cl₂ adduct (0.457 g, 0.559 mmol) and heating at 100° C. for 12 h. This gave after work up and crystallization using MeOH (20 mL) 2-(prop-1-en-2-yl)-N-(1-(3,4,5-trimethoxyphenyl)-1H-imidazol-4-yl)furo[3,2-d]pyrimidin-4-amine (62c) (0.6 g, 1.47 mmol) as an off-white solid; ¹H NMR (300 MHz, DMSO-d₆) δ 10.53 (s, 1H), 8.30 (d, J=2.2 Hz, 1H), 8.21 (s, 1H), 8.09 (d, J=1.3 Hz, 1H), 7.07 (d, J=2.2 Hz, 1H), 6.94 (s, 2H), 6.34 (s, 1H), 5.46 (s, 1H), 3.87 (s, 6H), 3.69 (s, 3H), 2.28 (s, 3H); MS (ES+): 408.4 (M+1), (ES−): 406.4 (M−1); Analysis calculated for C₂₁H₂₁N₅O₄·0.25H₂O: C, 61.23; H, 5.26; N, 17.00; Found: C, 61.21; H, 5.19; N, 17.04.

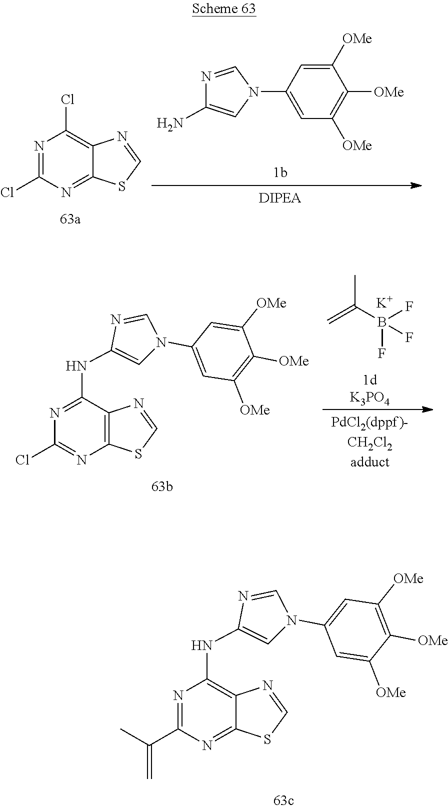

### Preparation of 5-(prop-1-en-2-yl)-N-(1-(3,4,5-trimethoxyphenyl)-1H-imidazol-4-yl)thiazolo[5,4-d]pyrimidin-7-amine (63c)

### Step-1: Preparation of 5-chloro-N-(1-(3,4,5-trimethoxyphenyl)-1H-imidazol-4-yl)thiazolo[5,4-d]pyrimidin-7-amine (63b)

Compound 63b was prepared according to the procedure reported in step-1 of scheme 1, from 5,7-dichlorothiazolo[5,4-d]pyrimidine (63a) (900 mg, 4.37 mmol; CAS #13479-88-4) in EtOH (2.0 mL) using DIPEA (2.3 mL, 13.11 mmol), 1-(3,4,5-trimethoxyphenyl)-1H-imidazol-4-amine (1b) (1.31 g, 5.26 mmol) and heating at 80° C. for 2 h. This gave after work up 5-chloro-N-(1-(3,4,5-trimethoxyphenyl)-1H-imidazol-4-yl)thiazolo[5,4-d]pyrimidin-7-amine (63b) (1.2 g, 66% yield) as a cream color solid; ¹H NMR (300 MHz, DMSO-d₆) δ 10.80 (s, 1H), 9.37 (s, 1H), 8.19 (d, J=1.5 Hz, 1H), 7.89 (s, 1H), 6.94 (s, 2H), 3.88 (s, 6H), 3.70 (s, 3H).

### Step-2: Preparation of 5-(prop-1-en-2-yl)-N-(1-(3,4,5-trimethoxyphenyl)-1H-imidazol-4-yl)thiazolo[5,4-d]pyrimidin-7-amine (63c)

Compound 63c was prepared according to the procedure reported in step-1 of scheme 1, from 5-chloro-N-(1-(3,4,5-trimethoxyphenyl)-1H-imidazol-4-yl)thiazolo[5,4-d]pyrimidin-7-amine (63b) (1.3 g, 3.1 mmol) in toluene (100 mL) using potassium isopropenyltrifluoroborate (1d)(0.92 g, 6.2 mmol), a solution of potassium phosphate (1.0 g, 4.66 mmol) in water (5 mL), PdCl₂(dppf)-CH₂Cl₂ adduct (380 mg, 0.465 mmol) and heating at 100° C. for 6 h. This gave after work up 5-(prop-1-en-2-yl)-N-(1-(3,4,5-trimethoxyphenyl)-1H-imidazol-4-yl)thiazolo[5,4-d]pyrimidin-7-amine (63c) (700 mg, 53% yield) as a cream color solid; ¹H NMR (300 MHz, DMSO-d₆) δ 10.24 (s, 1H, D₂O exchangeable), 9.34 (s, 1H), 8.22 (d, J=1.6 Hz, 1H), 8.11 (d, J=1.6 Hz, 1H), 6.95 (s, 2H), 6.49-6.38 (m, 1H), 5.59 (s, 1H), 3.88 (s, 6H), 3.69 (s, 3H), 2.30 (s, 3H); MS (ES+): 425.10 (M+1); Analysis calculated for: C₂₀H₂₀N₆O₃S·0.5H₂O: C, 56.00; H, 4.82; N, 19.59; Found: C, 55.89; H, 4.73; N, 19.32.

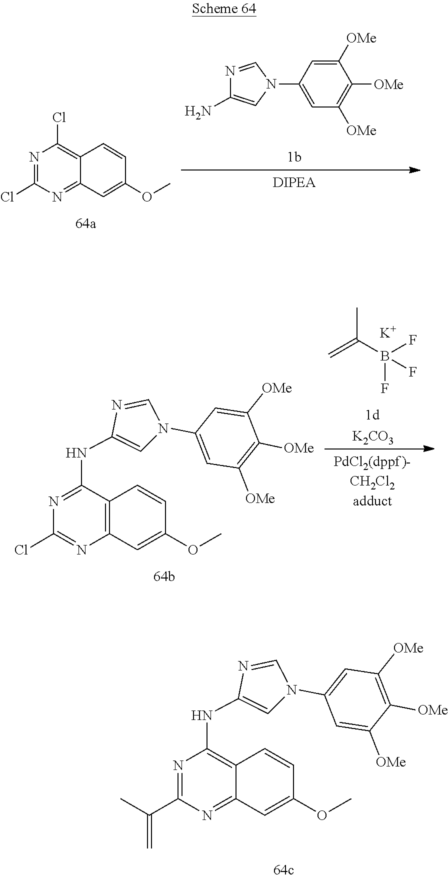

### Preparation of 7-methoxy-2-(prop-1-en-2-yl)-N-(1-(3,4,5-trimethoxyphenyl)-1H-imidazol-4-yl)quinazolin-4-amine (64c)

### Step-1: Preparation of 2-chloro-7-methoxy-N-(1-(3,4,5-trimethoxyphenyl)-1H-imidazol-4-yl)quinazolin-4-amine (64b)

Compound 64b was prepared according to the procedure reported in step-1 of scheme 1, from 2,4-dichloro-7-methoxyquinazoline (64a) (0.33 g, 1.44 mmol; CAS #62484-31-5) in EtOH (6.6 mL) and DCM (1.0 mL) using DIPEA (0.58 g, 4.32 mmol), 1-(3,4,5-trimethoxyphenyl)-1H-imidazol-4-amine (1b) (0.430 g, 1.73 mmol) and heating at 60° C. for 12 h. This gave after work up 2-chloro-7-methoxy-N-(1-(3,4,5-trimethoxyphenyl)-1H-imidazol-4-yl)quinazolin-4-amine (64b) (0.28 g, 44% yield) as a brown solid; ¹H NMR (300 MHz, DMSO-d₆) δ 10.90 (s, 1H), 8.65 (d, J=9.1 Hz, 1H), 8.21 (d, J=1.6 Hz, 1H), 7.96 (d, J=1.6 Hz, 1H), 7.27-7.10 (m, 2H), 6.94 (s, 2H), 3.92 (s, 3H), 3.89 (s, 6H), 3.71 (s, 3H).

### Step-2: Preparation of 7-methoxy-2-(prop-1-en-2-yl)-N-(1-(3,4,5-trimethoxyphenyl)-1H-imidazol-4-yl)quinazolin-4-amine (64c)

Compound 64c was prepared according to the procedure reported in step-1 of scheme 1, from 2-chloro-7-methoxy-N-(1-(3,4,5-trimethoxyphenyl)-1H-imidazol-4-yl)quinazolin-4-amine (64b) (0.28 g, 0.63 mmol) in 1,4-dioxane (11.2 mL) using potassium isopropenyltrifluoroborate (Id)(0.281 g, 1.90 mmol), a solution of potassium carbonate (0.262 g, 1.90 mmol) in water (2.0 mL), PdCl₂(dppf)-CH₂Cl₂ adduct (0.103 g, 0.126 mmol) and heating at 100° C. for 12 h. This gave after work up and purification using column chromatography [silica gel (24 g), eluting with MeOH in DCM from 0-5%] 7-methoxy-2-(prop-1-en-2-yl)-N-(1-(3,4,5-trimethoxyphenyl)-1H-imidazol-4-yl)quinazolin-4-amine (64c) (0.1 g, 35% yield) as an off-white solid; ¹H NMR (300 MHz, DMSO-d₆) δ 10.48 (s, 1H), 8.62 (d, J=9.1 Hz, 1H), 8.26 (d, J=1.6 Hz, 1H), 8.19 (d, J=1.6 Hz, 1H), 7.22-7.08 (m, 2H), 6.95 (s, 2H), 6.48 (d, J=2.9 Hz, 1H), 5.56 (s, 1H), 3.92 (s, 3H), 3.88 (s, 6H), 3.69 (s, 3H), 2.30 (s, 3H); MS (ES+): 448.20 (M+1); (ES−): 446.10 (M−1); Analysis calculated for: C₂₄H₂₅N₅O₄: C, 64.42; H, 5.63; N, 15.65; Found: C, 64.34; H, 5.66; N, 15.62.

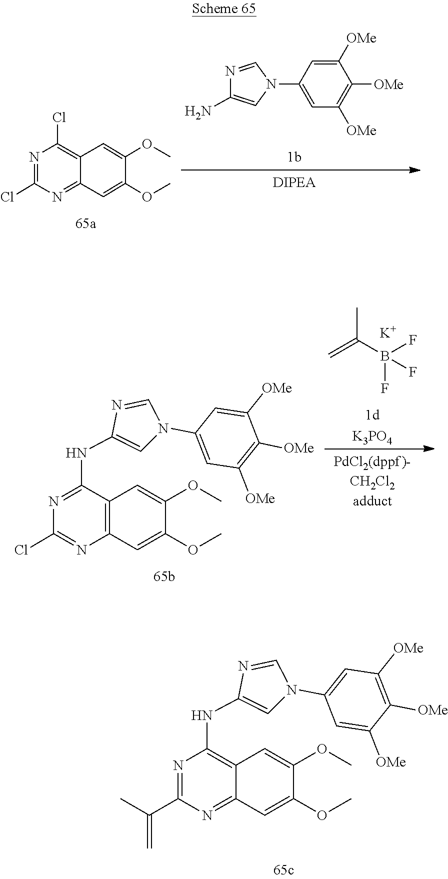

### Preparation of 6,7-dimethoxy-2-(prop-1-en-2-yl)-N-(1-(3,4,5-trimethoxyphenyl)-1H-imidazol-4-yl)quinazolin-4-amine (65c)

### Step-1: Preparation of 2-chloro-6,7-dimethoxy-N-(1-(3,4,5-trimethoxyphenyl)-1H-imidazol-4-yl)quinazolin-4-amine (65b)

Compound 65b was prepared according to the procedure reported in step-1 of scheme 1, from 2,4-dichloro-6,7-dimethoxyquinazoline (65a) (1.0 g, 3.86 mmol; CAS #27631-29-4) in EtOH (20 mL) and DCM (2 mL) using DIPEA (1.4 g, 11.57 mmol), 1-(3,4,5-trimethoxyphenyl)-1H-imidazol-4-amine (1b) (0.96 g, 3.85 mmol) and stirring at RT for 12 h. This gave afterwork up 2-chloro-6,7-dimethoxy-N-(1-(3,4,5-trimethoxyphenyl)-1H-imidazol-4-yl)quinazolin-4-amine (65b) (0.87 g, 48% yield) as a brown solid; ¹H NMR (300 MHz, DMSO-d₆) δ 10.85 (s, 1H), 8.21 (d, J=1.6 Hz, 1H), 8.13 (s, 1H), 7.96 (d, J=1.6 Hz, 1H), 7.17 (s, 1H), 6.94 (s, 2H), 3.98-3.85 (m, 12H), 3.70 (s, 3H).

### Step-2: Preparation of 6,7-dimethoxy-2-(prop-1-en-2-yl)-N-(1-(3,4,5-trimethoxyphenyl)-1H-imidazol-4-yl)quinazolin-4-amine (65c)

Compound 65c was prepared according to the procedure reported in step-1 of scheme 1, from 2-chloro-6,7-dimethoxy-N-(1-(3,4,5-trimethoxyphenyl)-1H-imidazol-4-yl)quinazolin-4-amine (65b) (0.7 g, 1.48 mmol) in 1,4-dioxane (21 mL) using potassium isopropenyltrifluoroborate (1d) (0.32 g, 2.96 mmol), a solution of potassium phosphate (0.94 g, 4.45 mmol) in water (2.1 mL), PdCl₂(dppf)-CH₂Cl₂ adduct (0.24 g, 0.29 mmol) and heating at 100° C. for 12 h. This gave after work up and purification using column chromatography [silica gel, eluting with MeOH in DCM from 0-5%] 6,7-dimethoxy-2-(prop-1-en-2-yl)-N-(1-(3,4,5-trimethoxyphenyl)-1H-imidazol-4-yl)quinazolin-4-amine (65c) (0.120 g, 17% yield) as an off-white solid; ¹H NMR (300 MHz, DMSO-d₄) δ 10.44 (s, 1H, D₂O exchangeable), 8.28 (d, J=1.6 Hz, 1H), 8.20 (d, J=1.6 Hz, 1H), 8.12 (s, 1H), 7.19 (s, 1H), 6.96 (s, 2H), 6.44 (d, J=2.9 Hz, 1H), 5.51 (s, 1H), 4.03-3.84 (m, 12H), 3.70 (s, 3H), 2.31 (s, 3H); MS (ES+): 478.20 (M+1); (ES−): 476.15 (M−1).

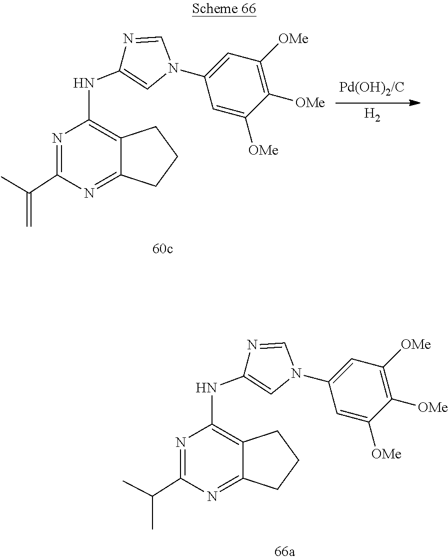 [SMILES: C=C(C)c1nc2c(c(Nc3cn(-c4cc(OC)c(OC)c(OC)c4)cn3)n1)CCC2 ; "H2" ; COc1cc(-n2cnc(Nc3nc(C(C)C)nc4c3CCC4)c2)cc(OC)c1OC]

### Preparation of 2-isopropyl-N-(1-(3,4,5-trimethoxyphenyl)-1H-imidazol-4-yl)-6,7-dihydro-5H-cyclopenta[d]pyrimidin-4-amine (66a)

To a stirred solution of 2-(prop-1-en-2-yl)-N-(1-(3,4,5-trimethoxyphenyl)-1H-imidazol-4-yl)-6,7-dihydro-5H-cyclopenta[d]pyrimidin-4-amine (60c) (0.25 g, 0.61 mmol) in MeOH (30 mL) and DCM (3 mL) was added 50% wet, 20% Pd(OH)₂ on carbon (0.065 g, 0.046 mmol) and stirred at RT for 16 h under hydrogen at atmospheric pressure. The reaction mixture was filtered through a pad of Celite, washed with 10% MeOH in DCM (50 mL) and the filtrate was concentrated in vacuum. The residue obtained was crystallized using diethyl ether (5 mL) to give 2-isopropyl-N-(1-(3,4,5-trimethoxyphenyl)-1H-imidazol-4-yl)-6,7-dihydro-5H-cyclopenta[d]pyrimidin-4-amine (66a) (40 mg, 16% yield) as an off white solid; ¹H NMR (300 MHz, DMSO-d₆) δ 9.40 (s, 1H, D₂O exchangeable), 8.16 (d, J=1.6 Hz, 1H), 8.04 (d, J=1.6 Hz, 1H), 6.89 (s, 2H), 3.86 (s, 6H), 3.68 (s, 3H), 3.06-2.93 (m, 1H), 2.89-2.68 (m, 4H), 2.07-1.91 (m, 2H), 1.30 (d, J=6.9 Hz, 6H); MS (ES+): 410.30 (M+1).

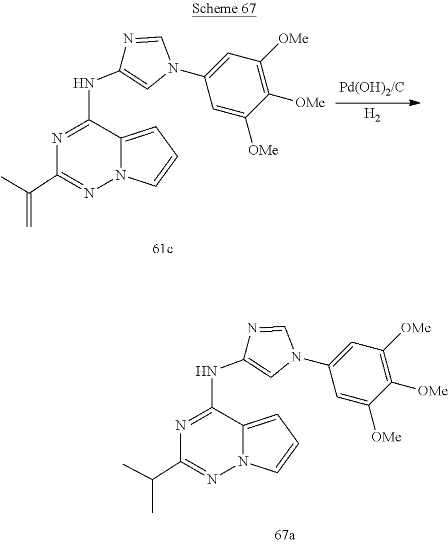 [SMILES: C=C(C)c1nc(Nc2cn(-c3cc(OC)c(OC)c(OC)c3)cn2)c2cccn2n1 ; "H2" ; COc1cc(-n2cnc(Nc3nc(C(C)C)nn4cccc34)c2)cc(OC)c1OC]

### Preparation of 2-isopropyl-N-(1-(3,4,5-trimethoxyphenyl)-1H-imidazol-4-yl)pyrrolo[2,1-f][1,2,4]triazin-4-amine (67a)

Compound 67a was prepared according to the procedure reported in scheme 66, from 2-(prop-1-en-2-yl)-N-(1-(3,4,5-trimethoxyphenyl)-1H-imidazol-4-yl)pyrrolo[2,1-f][1,2,4]triazin-4-amine (61c) (0.175 g, 0.43 mmol) in MeOH (10.5 mL) and DCM (3.5 mL) using 50% wet, 20% Pd(OH)₂ on carbon (0.045 g 0.032 mmol) and stirring at RT for 16 h under a hydrogen atmosphere. This gave after work up and recrystallization using diethyl ether (5.0 mL) 2-isopropyl-N-(1-(3,4,5-trimethoxyphenyl)-1H-imidazol-4-yl)pyrrolo[2,1-f][1,2,4]triazin-4-amine (67a) (90 mg, 51% yield) as an off white solid; ¹H NMR (300 MHz, DMSO-d₆) δ 10.76 (s, 1H), 8.22 (d, J=1.6 Hz, 1H), 8.12 (s, 1H), 7.65 (d, J=2.6 Hz, 1H), 7.24 (s, 1H), 6.93 (s, 2H), 6.63 (dd, J=4.3, 2.5 Hz, 1H), 3.87 (s, 6H), 3.70 (s, 3H), 2.95 (p, J=7.0 Hz, 1H), 1.33 (d, J=6.8 Hz, 6H); MS (ES+): 409.20 (M+1); (ES−): 407.20 (M−1).

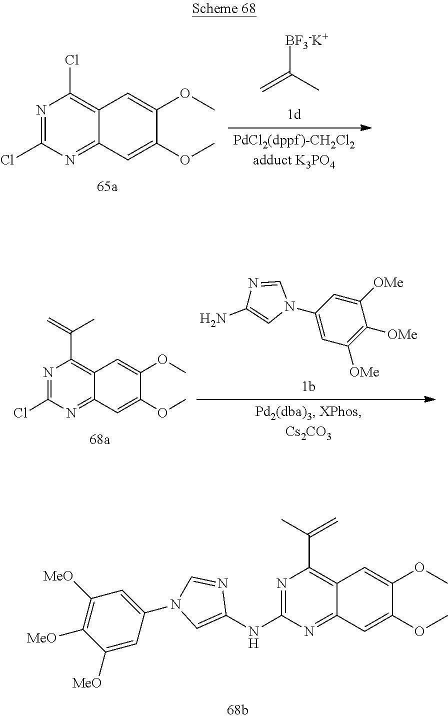

### Preparation of 6,7-dimethoxy-4-(prop-1-en-2-yl)-N-(1-(3,4,5-trimethoxyphenyl)-1H-imidazol-4-yl)quinazolin-2-amine (68b)

### Step-1: Preparation of 2-chloro-6,7-dimethoxy-4-(prop-1-en-2-yl)quinazoline (68a)

Compound 68a was prepared according to the procedure reported in step-1 of scheme 1, from 2,4-dichloro-6,7-dimethoxyquinazoline (65a) (1.0 g, 3.86 mmol) in toluene (15.0 mL) using potassium isopropenyltrifluoroborate (1d) (0.54 g, 5.01 mmol), a solution of potassium phosphate (2.45 g, 11.57 mmol) in water (3.0 mL), PdCl₂(dppf)-CH₂Cl₂ adduct (0.63 g, 0.77 mmol) and heating at 100° C. for 12 h. This gave after work up and purification using column chromatography [silica gel, eluting with MeOH in DCM from 0-5%] 2-chloro-6,7-dimethoxy-4-(prop-1-en-2-yl)quinazoline (68a) (0.12 g, 12% yield) as an off-white solid; 1H NMR (300 MHz, DMSO-d₆) δ 7.45 (s, 1H), 7.39 (s, 1H), 5.84-5.77 (m, 1H), 5.52 (s, 1H), 4.00 (s, 3H), 3.92 (s, 3H), 2.22 (s, 3H).

### Step-2: Preparation of 6,7-dimethoxy-4-(prop-1-en-2-yl)-N-(1-(3,4,5-trimethoxyphenyl)-1H-imidazol-4-yl)quinazolin-2-amine (68b)

Compound 68b was prepared according to the procedure reported in step-4 of scheme 27, from 2-chloro-6,7-dimethoxy-4-(prop-1-en-2-yl)quinazoline (68a) (0.220 g, 0.83 mmol) in 1,4-dioxane (4.4 mL) using 1-(3,4,5-trimethoxyphenyl)-1H-imidazol-4-amine (1b) (0.24 g, 0.96 mmol), Pd₂(dba)₃ (0.15 g, 0.166 mmol), X-Phos (0.15 g, 0.33 mmol), Cs₂CO3 (0.81 g, 2.48 mmol) and heating at 100° C. for 12 h. This gave after work up and purification using column chromatography [silica gel, eluting with MeOH in DCM from 0-5%] 6,7-dimethoxy-4-(prop-1-en-2-yl)-N-(1-(3,4,5-trimethoxyphenyl)-1H-imidazol-4-yl)quinazolin-2-amine (68b) (0.07 g, 17.67% yield) as an off white solid; ¹H NMR (300 MHz, DMSO-d₆) δ 9.57 (s, 1H, D₂O exchangeable), 8.06 (d, J=1.6 Hz, 1H), 8.04-7.97 (m, 1H), 7.29 (s, 1H), 7.24 (s, 1H), 6.93 (s, 2H), 5.70 (s, 1H), 5.40 (s, 1H), 3.95 (s, 3H), 3.90 (s, 6H), 3.83 (s, 3H), 3.69 (s, 3H), 2.24 (s, 3H); MS (ES+): 478.20 (M+1).

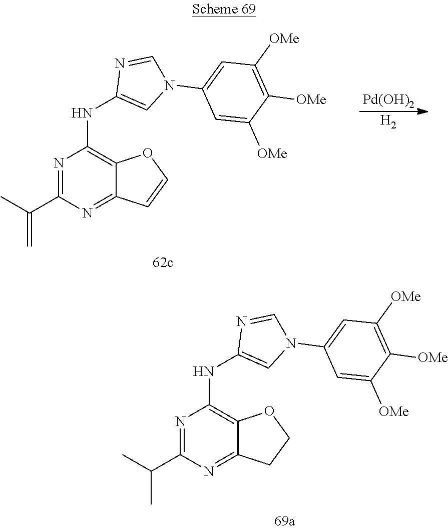

### Preparation of 2-isopropyl-N-(1-(3,4,5-trimethoxyphenyl)-1H-imidazol-4-yl)-6,7-dihydrofuro[3,2-d]pyrimidin-4-amine (69a)

Compound 69a was prepared according to the procedure reported in step-3 of scheme 1, from 2-(prop-1-en-2-yl)-N-(1-(3,4,5-trimethoxyphenyl)-1H-imidazol-4-yl)furo[3,2-d]pyrimidin-4-amine (62c) (0.25 g, 0.614 mmol) in MeOH:DCM (ratio 10:2, 20 mL) using 50% wet, 20% to Pd(OH)₂ on carbon (42 mg, 0.03 mmol) and stirring for 12 h at RT under a H₂ atmosphere. The reaction mixture was filtered through a pad of Celite, washed with 10% MeOH in DCM (10 mL) and the filtrate was concentrated in vacuum. The residue obtained was crystallized using MeOH (10 mL) to give 2-isopropyl-N-(1-(3,4,5-trimethoxyphenyl)-1H-imidazol-4-yl)-6,7-dihydrofuro[3,2-d]pyrimidin-4-amine (69a) (0.03 g, 12% yield) as an off white solid; ¹H NMR (300 MHz, DMSO-d₆) δ 9.33 (s, 1H, D₂O exchangeable), 8.11 (d, J=1.6 Hz, 1H), 7.99 (d, J=1.6 Hz, 1H), 6.88 (s, 2H), 4.60 (t, J=9.0 Hz, 2H), 3.86 (s, 6H), 3.68 (s, 3H), 3.19 (t, J=9.0 Hz, 2H), 3.09-2.92 (m, 1H), 1.29 (d, J=6.9 Hz, 6H); MS (ES+): 412.20 (M+1).

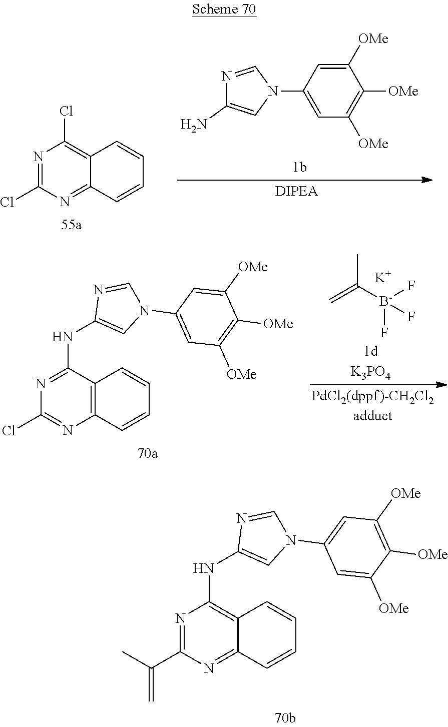

### Preparation of 2-(prop-1-en-2-yl)-N-(1-(3,4,5-trimethoxyphenyl)-H-imidazol-4-yl)quinazolin-4-amine (70b)

### Step-1: Preparation of 2-chloro-N-(1-(3,4,5-trimethoxyphenyl)-1H-imidazol-4-yl)quinazolin-4-amine (70a)

Compound 70a was prepared according to the procedure reported in step-1 of scheme 1, from 2,4-dichloroquinazoline (55a) (0.5 g, 2.51 mmol) in EtOH (15 mL) and DCM (1 mL) using DIPEA (1.073 g, 8.304 mmol), 1-(3,4,5-trimethoxyphenyl)-1H-imidazol-4-amine (1b) (0.828 g, 3.322 mmol) and stirring at RT for 12 h. This gave after work up 2-chloro-N-(1-(3,4,5-trimethoxyphenyl)-1H-imidazol-4-yl)quinazolin-4-amine (70a) (0.59 g, 57% yield) as a white solid; ¹H NMR (300 MHz, DMSO-d₆) δ 11.12 (s, 1H), 8.76 (d, J=8.2 Hz, 1H), 8.24 (d, J=1.6 Hz, 1H), 8.01 (d, J=1.6 Hz, 1H), 7.92-7.82 (m, 1H), 7.72 (d, J=8.2 Hz, 1H), 7.66-7.55 (m, 1H), 6.95 (s, 2H), 3.89 (s, 6H), 3.71 (s, 3H).

### Step-2: Preparation of 2-(prop-1-en-2-yl)-N-(1-(3,4,5-trimethoxyphenyl)-1H-imidazol-4-yl)quinazolin-4-amine (70b)

Compound 70b was prepared according to the procedure reported in step-1 of scheme 1, from 2-chloro-N-(1-(3,4,5-trimethoxyphenyl)-1H-imidazol-4-yl)quinazolin-4-amine (70a) (0.590 g, 1.43 mmol) in 1,4-dioxane (17.7 mL) using potassium isopropenyltrifluoroborate (1d)(0.418 g, 2.826 mmol), a solution of potassium phosphate (0.586 g, 4.239 mmol) in water (3 mL), PdCl₂(dppf)-CH₂C₁ adduct (0.230 g, 0.283 mmol) and heating at 100° C. for 12 h. This gave after work up and purification using column chromatography [silica gel (24 g), eluting with MeOH in DCM from 0-3%] 2-(prop-1-en-2-yl)-N-(1-(3,4,5-trimethoxyphenyl)-1H-imidazol-4-yl)quinazolin-4-amine (70b) (0.2 g, 34%) as a pale yellow solid; ¹H NMR (300 MHz, DMSO-d₆) δ 10.64 (s, 1H), 8.72 (d, J=8.4 Hz, 1H), 8.28 (d, J=1.5 Hz, 1H), 8.23 (d, J=1.6 Hz, 1H), 7.89-7.70 (m, 2H), 7.60-7.44 (m, 1H), 6.97 (s, 2H), 6.50 (d, J=2.7 Hz, 1H), 5.59 (s, 1H), 3.88 (s, 6H), 3.70 (s, 3H), 2.31 (s, 3H); MS (ES+): 418.20 (M+1); (ES−): 416.20 (M−1).

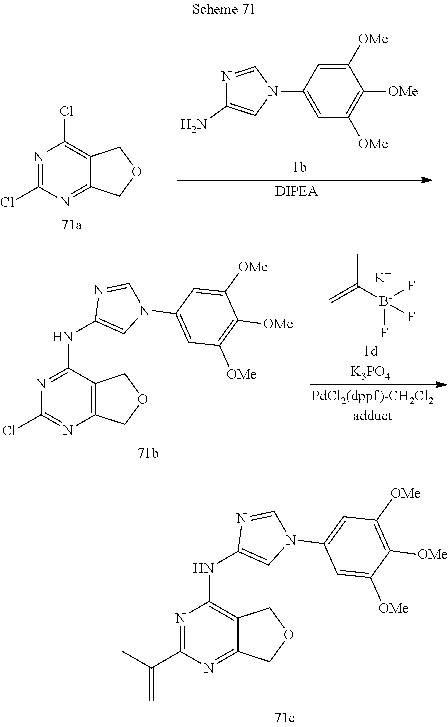

### Preparation of 2-(prop-1-en-2-yl)-N-(1-(3,4,5-trimethoxyphenyl)-1H-imidazol-4-yl)-5,7-dihydrofuro[3,4-d]pyrimidin-4-amine (71c)

### Step-1: Preparation of 2-chloro-N-(1-(3,4,5-trimethoxyphenyl)-1H-imidazol-4-yl)-5,7-dihydrofuro[3,4-d]pyrimidin-4-amine (71b)

Compound 71b was prepared according to the procedure reported in step-1 of scheme 1, from 2,4-dichloro-5,7-dihydrofuro[3,4-d]pyrimidine (71a) (0.8 g, 4.19 mmol; CAS #848398-41-4) in EtOH (24 mL) using DIPEA (1.623 g, 12.564 mmol), 1-(3,4,5-trimethoxyphenyl)-1H-imidazol-4-amine (1b) (1.25 g, 5.03 mmol) and stirring at RT for 12 h. This gave after work up 2-chloro-N-(1-(3,4,5-trimethoxyphenyl)-1H-imidazol-4-yl)-5,7-dihydrofuro[3,4-d]pyrimidin-4-amine (71b) (0.450 g, 27%) as a brown solid; ¹H NMR (300 MHz, DMSO-d₆) δ 10.41 (s, 1H), 8.16 (s, 1H), 7.78 (d, J=1.6 Hz, 1H), 6.91 (s, 2H), 4.96 (s, 2H), 4.83 (s, 2H), 3.87 (s, 6H), 3.69 (s, 3H).

### Step-2: Preparation of 2-(prop-1-en-2-yl)-N-(1-(3,4,5-trimethoxyphenyl)-1H-imidazol-4-yl)-5,7-dihydrofuro[3,4-d]pyrimidin-4-amine (71c)

Compound 71c was prepared according to the procedure reported in step-1 of scheme 1, from 2-chloro-N-(1-(3,4,5-trimethoxyphenyl)-1H-imidazol-4-yl)-5,7-dihydrofuro[3,4-d]pyrimidin-4-amine (71b) (0.45 g, 1.11 mmol) in 1,4-dioxane (13.5 mL) using potassium isopropenyltrifluoroborate (1d) (0.412 g, 2.785 mmol), potassium phosphate (0.709 g, 3.342 mmol), PdCl₂(dppf)-CH₂Cl₂ adduct (0.182 g, 0.223 mmol) and heating at 100° C. for 12 h. This gave after work up and purification using column chromatography [silica gel (24 g), eluting with MeOH in DCM from 0-2%] 2-(prop-1-en-2-yl)-N-(1-(3,4,5-trimethoxyphenyl)-1H-imidazol-4-yl)-5,7-dihydrofuro[3,4-d]pyrimidin-4-amine (71c) (0.190 g, 42%) as an off-white solid; ¹H NMR (300 MHz, DMSO-d₆) δ 10.01 (s, 1H D₂O exchangeable), 8.21 (d, J=1.5 Hz, 1H), 8.02 (d, 1H), 6.92 (s, 2H), 6.43-6.31 (m, 1H), 5.50 (s, 1H), 5.03 (s, 2H), 4.86 (s, 2H), 3.86 (s, 6H), 3.68 (s, 3H), 2.26 (s, 3H); MS (ES+): 410.20 (M+1); (ES−): 408.20 (M−1)

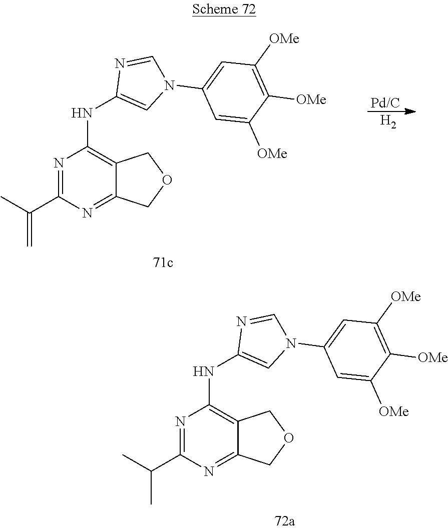 [SMILES: C=C(C)c1nc2c(c(Nc3cn(-c4cc(OC)c(OC)c(OC)c4)cn3)n1)COC2 ; "Pd/C" ; "H2" ; COc1cc(-n2cnc(Nc3nc(C(C)C)nc4c3COC4)c2)cc(OC)c1OC]

### Preparation of 2-isopropyl-N-(1-(3,4,5-trimethoxyphenyl)-1H-imidazol-4-yl)-5,7-dihydrofuro[3,4-d]pyrimidin-4-amine (72a)

Compound 72a was prepared according to the procedure reported in scheme 41, from 2-(prop-1-en-2-yl)-N-(1-(3,4,5-trimethoxyphenyl)-1H-imidazol-4-yl)-5,7-dihydrofuro[3,4-d]pyrimidin-4-amine (71c) (0.14 g, 0.342 mmol) in ethanol (8.4 mL) and DCM (4.2 mL) using 50% wet, 10% Pd/C (0.145 g, 0.068 mmol) and stirring at RT for 4 h under a H₂ atmosphere. This gave after work up and purification by column chromatography [silica gel, eluting with MeOH in DCM from 0-3%] 2-isopropyl-N-(1-(3,4,5-trimethoxyphenyl)-1H-imidazol-4-yl)-5,7-dihydrofuro[3,4-d]pyrimidin-4-amine (72a) (0.082 g, 58% yield) as an off white solid; ¹H NMR (300 MHz, DMSO-d₆) δ 9.91 (s, 11H, D₂O exchangeable), 8.18 (d, J=1.6 Hz, 1H), 8.03 (d, J=1.6 Hz, 1H), 6.90 (s, 2H), 4.98 (s, 21H), 4.82 (s, 2H), 3.86 (s, 6H), 3.68 (s, 3H), 3.14-2.98 (m, 1H), 1.32 (d, J=6.9 Hz, 6H); MS (ES+): 412.20 (M+1).

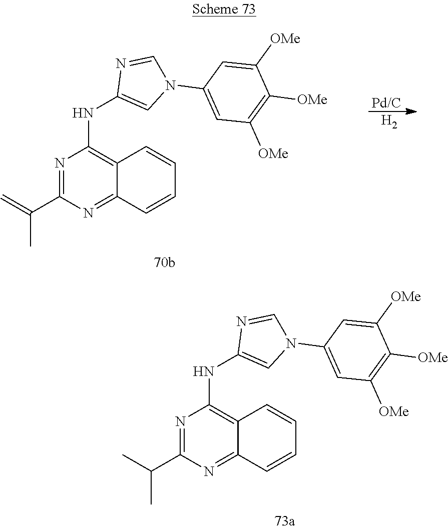 [SMILES: C=C(C)c1nc(Nc2cn(-c3cc(OC)c(OC)c(OC)c3)cn2)c2ccccc2n1 ; "Pd/C" ; "H2" ; COc1cc(-n2cnc(Nc3nc(C(C)C)nc4ccccc34)c2)cc(OC)c1OC]

### Preparation of 2-isopropyl-N-(1-(3,4,5-trimethoxyphenyl)-1H-imidazol-4-yl)quinazolin-4-amine (73a)

Compound 73a was prepared according to the procedure reported in scheme 41, from 2-(prop-1-en-2-yl)-N-(1-(3,4,5-trimethoxyphenyl)-1H-imidazol-4-yl)quinazolin-4-amine (70b) (0.14 g, 0.335 mmol) in ethanol (8.4 mL) and DCM (4.2 mL) using 50% wet 10% Pd/C (0.143 g, 0.067 mmol) and stirring at RT for 4 h under a H₂ atmosphere. This gave after work up and purification by column chromatography [silica gel, eluting with MeOH in DCM from 0-3%] 2-isopropyl-N-(1-(3,4,5-trimethoxyphenyl)-1H-imidazol-4-yl)quinazolin-4-amine (73a) (0.035 g, 25% yield) as an off white solid; ¹H NMR (300 MHz, DMSO-d) δ 10.62 (s, 1H, D₂O exchangeable), 8.70 (d, J=8.5 Hz, 1H), 8.33-8.22 (m, 2H), 7.87-7.67 (m, 2H), 7.56-7.42 (m, 1H), 6.96 (s, 2H), 3.89 (s, 6H), 3.71 (s, 3H), 3.22-3.06 (m, 1H), 1.40 (d, J=6.9 Hz, 6H); MS (ES+): 420.20 (M+1).

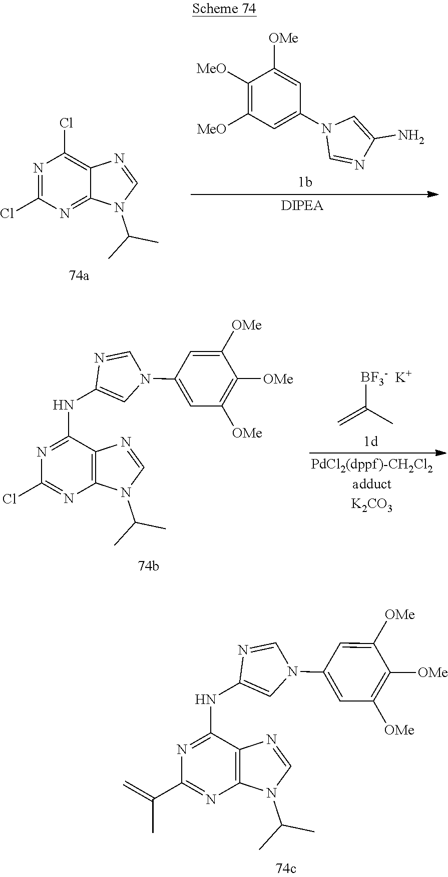

### Preparation of 9-isopropyl-2-(prop-1-en-2-yl)-N-(1-(3,4,5-trimethoxyphenyl)-1H-imidazol-4-yl)-9H-purin-6-amine (74c)

### Step-1: Preparation of 2-chloro-9-isopropyl-N-(1-(3,4,5-trimethoxyphenyl)-1H-imidazol-4-yl)-9H-purin-6-amine (74b)

Compound 74b was prepared according to the procedure reported in step-1 of scheme 1, from 2,6-dichloro-9-isopropyl-9H-purine (74a) (1.0 g, 4.33 mmol; CAS #20343645-7) in EtOH (20 mL) and DCM (2 mL) using DIPEA (1.67 g, 12.98 mmol), 1-(3,4,5-trimethoxyphenyl)-1H-imidazol-4-amine (1b) (1.29 g, 5.18 mmol) and heating at reflux for 12 h. This gave after work up 2-chloro-9-isopropyl-N-(1-(3,4,5-trimethoxyphenyl)-1H-imidazol-4-yl)-9H-purin-6-amine (74b) (0.8 g, 42% yield) as a brown solid; ¹H NMR (300 MHz. DMSO-d₆) δ 10.43 (s, 1H), 8.41 (s, 1H), 8.14 (d, J=1.6 Hz, 1H), 7.84 (d, J=1.6 Hz, 1H), 6.92 (s, 2H), 4.91-4.59 (m, 1H), 3.88 (s, 6H), 3.70 (s, 3H), 1.53 (d, J=6.7 Hz, 6H).

### Step-2: Preparation of 9-isopropyl-2-(prop-1-en-2-yl)-N-(1-(3,4,5-trimethoxyphenyl)-1H-imidazol-4-yl)-9H-purin-6-amine (74c)

Compound 74c was prepared according to the procedure reported in step-1 of scheme 1, from 2-chloro-9-isopropyl-N-(1-(3,4,5-trimethoxyphenyl)-1H-imidazol-4-yl)-9H-purin-6-amine (74b) (0.8 g, 1.8 mmol) in 1,4-dioxane/H₂O (24 mL) using potassium isopropenyltrifluoroborate (1d) (0.39 g, 3.62 mmol), a solution of potassium carbonate (0.75 g, 5.44 mmol) in water (2.4 mL), PdCl₂(dppf)-CH₂Cl₂ adduct (0.29 g, 0.36 mmol) and heating for 12 h at 100° C. This gave after work up and purification using column chromatography [silica gel, eluting with MeOH in DCM from 0-5%] 9-isopropyl-2-(prop-1-en-2-yl)-N-(1-(3,4,5-trimethoxyphenyl)-1H-imidazol-4-yl)-9H-purin-6-amine (74c) (0.35 g, 43% yield) as an off white solid; ¹H NMR (300 MHz, DMSO-d₆) δ 9.89 (s, 1H, D₂O exchangeable), 8.38 (s, 1H), 8.19 (d, J=1.6 Hz, 1H), 8.09 (d, J=1.6 Hz, 1H), 6.93 (s, 2H), 6.38 (d, J=2.8 Hz, 1H), 5.47 (s, 1H), 4.91-4.73 (m, 1H), 3.88 (s, 6H), 3.69 (s, 3H), 2.30 (s, 3H), 1.58 (d, J=6.7 Hz, 6H); MS (ES+): 450.25 (M+1); Calculated for C₂₃H₂₇N₇O₃·0.5 (H₂O): C, 60.25; H, 6.16; N, 21.38; Found: C, 60.40; H, 6.08; N, 21.05.

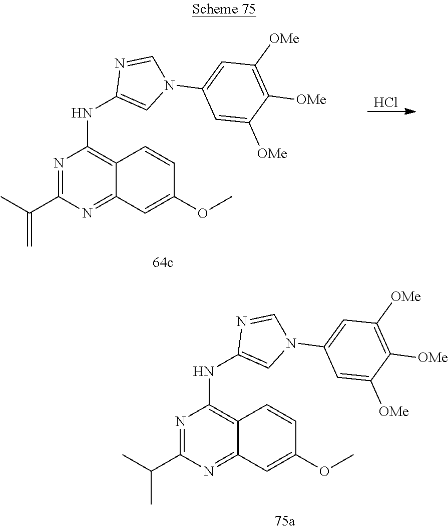 [SMILES: C=C(C)c1nc(Nc2cn(-c3cc(OC)c(OC)c(OC)c3)cn2)c2ccc(OC)cc2n1 ; Cl ; COc1ccc2c(Nc3cn(-c4cc(OC)c(OC)c(OC)c4)cn3)nc(C(C)C)nc2c1]

### Preparation of 2-isopropyl-7-methoxy-N-(1-(3,4,5-trimethoxyphenyl)-1H-imidazol-4-yl)quinazolin-4-amine (75a)

Compound 75a was prepared according to the procedure reported in scheme 41, from 7-methoxy-2-(prop-1-en-2-yl)-N-(1-(3,4,5-trimethoxyphenyl)-1H-imidazol-4-yl)quinazolin-4-amine (64c) (0.05 g, 0.11 mmol) in methanol (10 mL) and DCM (1 mL) using 50% wet 10% Pd/C (0.031 g, 0.022 mmol) and stirring at RT for 16 h under a H₂ atmosphere. This gave after work up and crystallization using diethyl ether (5.0 mL) 2-isopropyl-7-methoxy-N-(1-(3,4,5-trimethoxyphenyl)-1H-imidazol-4-yl)quinazolin-4-amine (75a) (45 mg, 91.8% yield) as a yellow solid; ¹H NMR (300 MHz, DMSO-d₆) δ 8.71 (s, 1H), 8.32 (s, 1H), 8.23 (s, 1H), 7.22 (s, 2H), 6.96 (s, 2H), 3.94 (s, 3H), 3.88 (s, 6H), 3.70 (s, 3H), 3.26-3.09 (m, 1H), 1.42 (d, J=6.8 Hz, 6H); MS (ES+): 450.4 (M+1); (ES−): 448.3.

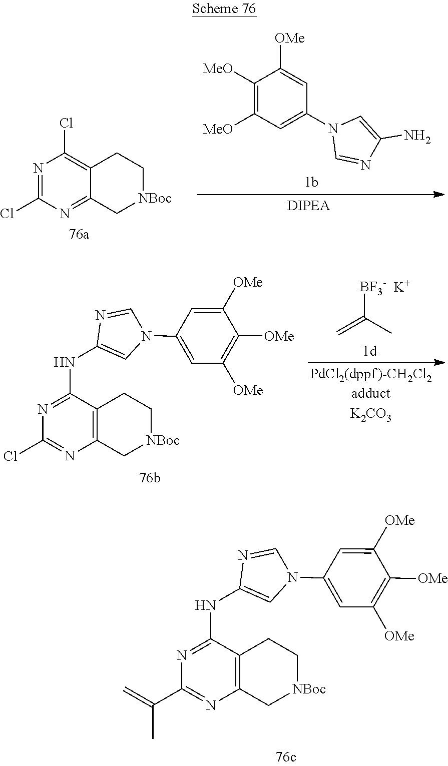

### Preparation of tert-butyl 2-(prop-1-en-2-yl)-4-((1-(3,4,5-trimethoxyphenyl)-1H-imidazol-4-yl)amino)-5,6-dihydropyrido[3,4-d]pyrimidine-7 (8H)-carboxylate (76c)

### Step-1: Preparation of tert-butyl 2-chloro-4-((1-(3,4,5-trimethoxyphenyl)-1H-imidazol-4-yl)amino)-5,6-dihydropyrido[3,4-d]pyrimidine-7 (8H)-carboxylate (76b)

Compound 76b was prepared according to the procedure reported in step-1 of scheme 1, from tert-butyl 2,4-dichloro-5,6-dihydropyrido[3,4-d]pyrimidine-7 (8H)-carboxylate (76a) (1.0 g, 3.29 mmol; CAS #916420-27-4) in EtOH (20 mL) and DCM (2 mL) using DIPEA (1.27 g, 9.86 mmol), 1-(3,4,5-trimethoxyphenyl)-1H-imidazol-4-amine (1b) (0.983 g, 3.94 mmol) and stirring at RT for 12 h. This gave after work up and purification using column chromatography [silica gel, eluting with MeOH in DCM from 0-5%] tert-butyl 2-chloro-4-((1-(3,4,5-trimethoxyphenyl)-1H-imidazol-4-yl)amino)-5,6-dihydropyrido[3,4-d]pyrimidine-7 (8H)-carboxylate (76b) (0.5 g, 29% yield) as a brown solid; ¹H NMR (300 MHz, DMF-d₇) S 9.68 (s, 1H), 8.16 (d, J=1.6 Hz, 1H), 7.79 (d, J=1.6 Hz, 1H), 6.90 (s, 2H), 4.35 (s, 2H), 3.87 (s, 6H), 3.69 (s, 3H), 3.67-3.53 (m, 2H), 2.73-2.61 (m, 2H), 1.43 (s, 9H).

### Step-2: Preparation of tert-butyl 2-(prop-1-en-2-yl)-4-((1-(3,4,5-trimethoxyphenyl)-1H-imidazol-4-yl)amino)-5,6-dihydropyrido[3,4-d]pyrimidine-7 (8H)-carboxylate (76c)

Compound 76c was prepared according to the procedure reported in step-1 of scheme 1, from tert-butyl 2-chloro-4-((1-(3,4,5-trimethoxyphenyl)-1H-imidazol-4-yl)amino)-5,6-dihydropyrido[3,4-d]pyrimidine-7 (8H)-carboxylate (76b) (0.2 g, 0.39 mmol) in 1,4-dioxane (10.0 mL) using potassium isopropenyltrifluoroborate (1d) (0.114 g, 0.77 mmol), a solution of potassium carbonate (0.16 g, 1.60 mmol) in water (2.0 mL). PdCl₂(dppf)-CH₂Cl₂ adduct (0.064 g, 0.077 mmol) and heating at 100° C. for 12 h. This gave after work up and purification using column chromatography [silica gel, eluting with MeOH in DCM from 0-5%] tert-butyl 2-(prop-1-en-2-yl)-4-((1-(3,4,5-trimethoxyphenyl)-1H-imidazol-4-yl)amino)-5,6-dihydropyrido[3,4-d]pyrimidine-7 (8H)-carboxylate (76c) (90 mg, 44.2% yield) as an off white solid; ¹H NMR (300 MHz, DMSO-d₆) δ 9.28 (s, 1H, D₂O exchangeable), 8.22 (d, J=1.6 Hz, 1H), 8.04 (d, J=1.6 Hz, 1H), 6.91 (s, 2H), 6.31 (d, J=2.7 Hz, 1H), 5.47 (s, 1H), 4.39 (s, 2H), 3.86 (s, 6H), 3.68 (s, 3H), 3.67-3.53 (m, 2H), 2.78-2.60 (m, 2H), 2.23 (s, 3H), 1.44 (s, 9H), MS (ES+): 523.30 (M+1).

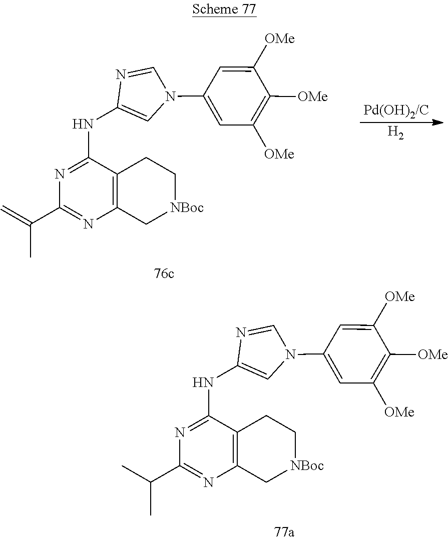 [SMILES: C=C(C)c1nc2c(c(Nc3cn(-c4cc(OC)c(OC)c(OC)c4)cn3)n1)CCN(C(=O)OC(C)(C)C)C2 ; "Pd(OH)2/C" ; "H2" ; COc1cc(-n2cnc(Nc3nc(C(C)C)nc4c3CCN(C(=O)OC(C)(C)C)C4)c2)cc(OC)c1OC]

### Preparation of tert-butyl 2-isopropyl-4-((1-(3,4,5-trimethoxyphenyl)-1H-imidazol-4-yl)amino)-5,6-dihydropyrido[3,4-d]pyrimidine-7 (8H)-carboxylate (77a)

Compound 77a was prepared according to the procedure reported in scheme 66, from tert-butyl 2-(prop-1-en-2-yl)-4-((1-(3,4,5-trimethoxyphenyl)-1H-imidazol-4-yl)amino)-5,6-dihydropyrido[3,4-d]pyrimidine-7 (8H)-carboxylate (76c) (0.15 g, 0.29 mmol) in MeOH (9 mL) and DCM (0.9 mL) using 20% Pd(OH)₂ on carbon (0.04 g, 0.028 mmol) and stirring at RT for 16 h under a H₂ atmosphere. This gave after work up and recrystallization using diethyl ether (5 mL) tert-butyl 2-isopropyl-4-((1-(3,4,5-trimethoxyphenyl)-1H-imidazol-4-yl)amino)-5,6-dihydropyrido[3,4-d]pyrimidine-7 (8H)-carboxylate (77a) (70 mg, 46% yield) as a brown solid; ¹H NMR (300 MHz, DMSO-d₆) δ 9.19 (s, 1H, D₂O exchangeable), 8.19 (s, 1H), 8.07 (d, J=1.6 Hz, 1H), 6.90 (s, 2H), 4.35 (s, 2H), 3.87 (s, 6H), 3.69 (s, 3H), 3.66-3.52 (m, 2H), 3.10-2.88 (m, 1H), 2.72-2.60 (m, 2H), 1.44 (s, 9H), 1.31 (d, J=6.8 Hz, 6H); MS (ES+): 525.30 (M+1).

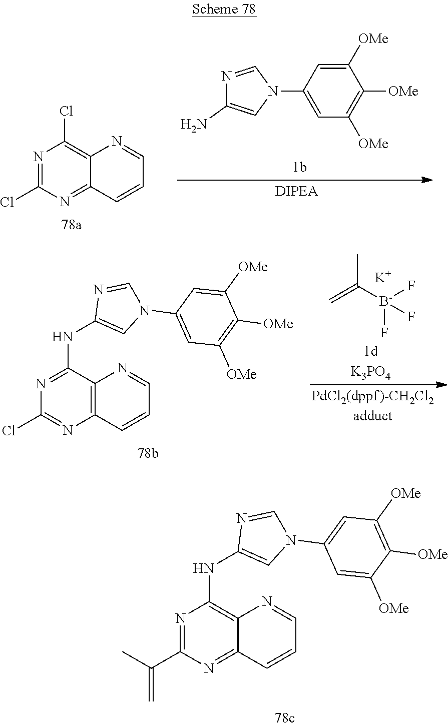

### Preparation of 2-(prop-1-en-2-yl)-N-(1-(3,4,5-trimethoxyphenyl)-1H-imidazol-4-yl)pyrido[3,2-d]pyrimidin-4-amine (78c)

### Step-1: Preparation of 2-chloro-N-(1-(3,4,5-trimethoxyphenyl)-1H-imidazol-4-yl)pyrido[3,2-d]pyrimidin-4-amine (78b)

Compound 78b was prepared according to the procedure reported in step-1 of scheme 1, from 2,4-dichloropyrido[3,2-d]pyrimidine (78a) (0.25 g, 1.25 mmol; CAS #39551-54-7) in EtOH (15 mL) using DIPEA (0.484 g, 3.75 mmol) and 1-(3,4,5-trimethoxyphenyl)-1H-imidazol-4-amine (1b) (0.374 g, 1.5 mmol) and stirring at RT for 12 h. This gave after work up 2-chloro-N-(1-(3,4,5-trimethoxyphenyl)-1H-imidazol-4-yl)pyrido[3,2-d]pyrimidin-4-amine (78b) (0.36 g, 73% yield) as a yellow solid; ¹H NMR (300 MHz, Chloroform-d) δ 9.61 (s, 1H), 8.79 (dd, J=4.3, 1.6 Hz, 1H), 8.09 (dd. J=8.5, 1.5 Hz, 1H), 8.00 (d, J=1.6 Hz, 1H), 7.72 (dd, J=8.5, 4.3 Hz, 1H), 7.66 (d, J=1.6 Hz, 1H), 6.68 (s, 2H), 3.95 (s, 6H), 3.89 (s, 3H).

### Step-2: Preparation of 2-(prop-1-en-2-yl)-N-(1-(3,4,5-trimethoxyphenyl)-1H-imidazol-4-yl)pyrido[3,2-d]pyrimidin-4-amine (78c)

Compound 78c was prepared according to the procedure reported in step-1 of scheme 1, from 2-chloro-N-(1-(3,4,5-trimethoxyphenyl)-1H-imidazol-4-yl)pyrido[3,2-d]pyrimidin-4-amine (78b) (0.36 g, 0.872 mmol) in 1,4-dioxane (10.8 mL) using potassium isopropenyltrifluoroborate (1d) (0.387 g, 2.616 mmol), potassium phosphate (0.370 g, 1.744 mmol), PdCl₂(dppf)-CH₂Cl₂ adduct (0.107 g, 0.131 mmol) and heating at 110° C. for 12 h under a nitrogen atmosphere. This gave after work up and purification using column chromatography [silica gel, eluting with MeOH in DCM from 0-2%] 2-(prop-1-en-2-yl)-N-(1-(3,4,5-trimethoxyphenyl)-1H-imidazol-4-yl)pyrido[3,2-d]pyrimidin-4-amine (78c) (0.220 g, 60%) as an bright yellow; ¹H NMR (300 MHz, DMSO-d₆) δ 9.74 (s, 1H, D₂O exchangeable), 8.89 (dd. J=4.3, 1.5 Hz, 1H), 8.25 (d, J=1.5 Hz, 11H), 8.22 (d, J=1.5 Hz, 1H), 8.14 (d, J=1.6 Hz, 1H), 7.90 (dd, J=8.5, 4.3 Hz, 1H), 6.97 (s, 2H), 6.55 (s, 1H), 5.65 (s, 1H), 3.88 (s, 6H), 3.70 (s, 3H), 2.31 (s, 3H); MS (ES+): 419.20 (M+1).

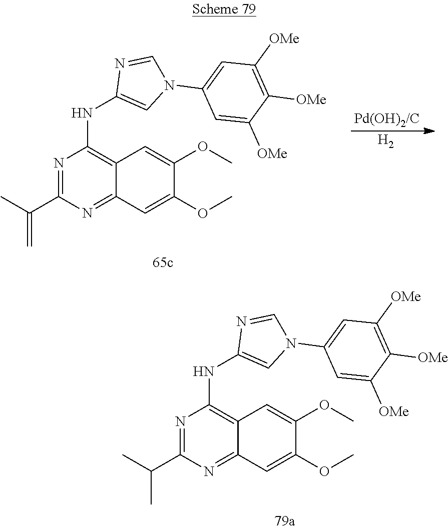 [SMILES: C=C(C)c1nc(Nc2cn(-c3cc(OC)c(OC)c(OC)c3)cn2)c2cc(OC)c(OC)cc2n1 ; "Pd(OH)2/C" ; "H2" ; COc1cc2nc(C(C)C)nc(Nc3cn(-c4cc(OC)c(OC)c(OC)c4)cn3)c2cc1OC]

### Preparation of 2-isopropyl-6,7-dimethoxy-N-(1-(3,4,5-trimethoxyphenyl)-1H-imidazol-4-yl)quinazolin-4-amine (79a)

Compound 79a was prepared according to the procedure reported in step-3 of scheme 1, from 6,7-dimethoxy-2-(prop-1-en-2-yl)-N-(1-(3,4,5-trimethoxyphenyl)-1H-imidazol-4-yl)quinazolin-4-amine (65c) (0.12 g, 0.25 mmol) in MeOH (14.4 mL) and DCM (1.44 mL) using 50% wet, 20% Pd(OH)? on carbon (0.031 g, 0.022 mmol) and stirring for 16 h at RT under a H₂ atmosphere. This gave after work up and recrystallization using diethyl ether (5 mL) 2-isopropyl-6,7-dimethoxy-N-(1-(3,4,5-trimethoxyphenyl)-1H-imidazol-4-yl)quinazolin-4-amine (79a) (37 mg, 31% yield) as an off white solid; ¹H NMR (300 MHz, DMSO-d₆) δ 10.41 (s, 1H), 8.25 (s, 2H), 8.08 (s, 1H), 7.14 (s, 1H), 6.95 (s, 2H), 4.02-3.82 (m, 12H), 3.70 (s, 3H), 3.17-2.99 (m, 1H), 1.38 (d, J=6.9 Hz, 6H); MS (ES+): 480.20 (M+1); Analysis calculated for C₂₅H₂₉N₅O₅·1.25H₂O: C, 59.81; H, 6.32; N, 13.95; Found: C, 60.08; H, 6.13; N, 13.59.

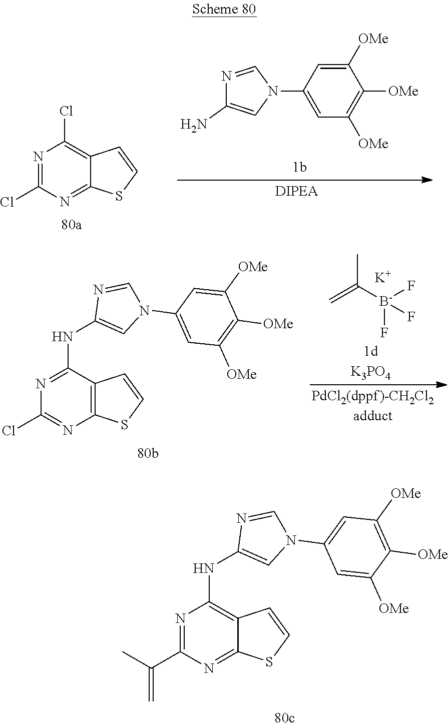

### Preparation of 2-(prop-1-en-2-yl)-N-(1-(3,4,5-trimethoxyphenyl)-1H-imidazol-4-yl)thieno[2,3-d]pyrimidin-4-amine (80c)

### Step-1: Preparation of 2-chloro-N-(1-(3,4,5-trimethoxyphenyl)-1H-imidazol-4-yl)thieno[2,3-d]pyrimidin-4-amine (80b)

Compound 80b was prepared according to the procedure reported in step-1 of scheme 1, from 2,4-dichlorothieno[2,3-d]pyrimidine (80a) (1.0 g, 4.88 mmol; CAS #18740-39-1) in IPA (100 mL) using DIPEA (1.47 g, 14.61 mmol), 1-(3,4,5-trimethoxyphenyl)-1H-imidazol-4-amine (1b) (1.45 g, 5.82 mmol) and heating at 85° C. for 12 h. This gave after work up 2-chloro-N-(1-(3,4,5-trimethoxyphenyl)-1H-imidazol-4-yl)thieno[2,3-d]pyrimidin-4-amine (80b) (1.08 g, 53% yield) as a cream colored solid; ¹H NMR (300 MHz, DMSO-d₆) δ 10.99 (s, 1H), 8.20 (d, J=1.6 Hz, 1H), 8.02 (s, 1H), 7.91 (d, J=1.6 Hz, 1H), 7.71 (d, J=5.9 Hz, 1H), 6.94 (s, 2H), 3.88 (s, 6H), 3.70 (s, 3H).

### Step-2: Preparation of 2-(prop-1-en-2-yl)-N-(1-(3,4,5-trimethoxyphenyl)-1H-imidazol-4-yl)thieno[2,3-d]pyrimidin-4-amine (80c)

Compound 80c was prepared according to the procedure reported in step-1 of scheme 1, from 2-chloro-N-(1-(3,4,5-trimethoxyphenyl)-1H-imidazol-4-yl)thieno[2,3-d]pyrimidin-4-amine (80b) (1.0 g, 2.39 mmol) in toluene (50 mL) using potassium isopropenyltrifluoroborate (1d) (0.71 g, 4.80 mmol), potassium phosphate (0.76 g, 3.58 mmol), PdCl₂(dppf)-CH₂Cl₂ adduct (196 mg, 0.24 mmol) and heating at 100° C. for 15 h under a nitrogen atmosphere. This gave after work up and purification using column chromatography [silica gel, eluting with methanolic ammonia in DCM from 2-5%] 2-(prop-1-en-2-yl)-N-(1-(3,4,5-trimethoxyphenyl)-1H-imidazol-4-yl)thieno[2,3-d]pyrimidin-4-amine (80c) (0.72 g, 71% yield) as a white solid; ¹H NMR (300 MHz, DMSO-d₆) δ 10.59 (s, 1H), 8.27 (d, J=1.6 Hz, 1H), 8.15 (d, J=1.6 Hz, 1H), 8.04 (d, J=6.0 Hz, 1H), 7.66 (d, J=6.0 Hz, 1H), 6.96 (s, 2H), 6.44 (d, J=2.7 Hz, 1H), 5.55 (s, 1H), 3.88 (s, 6H), 3.70 (s, 3H), 2.31 (s, 3H); MS (ES+): 424.10 (M+1); Analysis calculated for: C₂₁H₂₁N₅O₃S: C, 59.56; H, 5.00; N, 16.54; Found: C, 59.90; H, 4.93; N, 16.36.

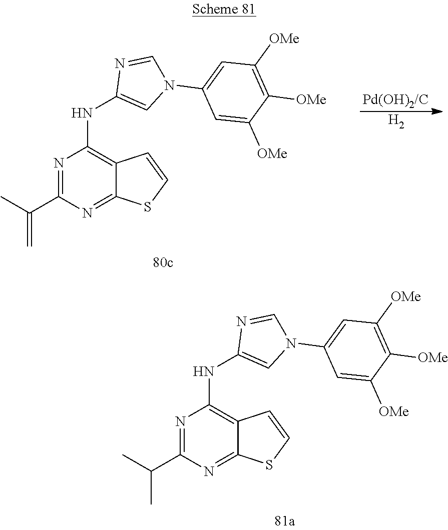 [SMILES: C=C(C)c1nc(Nc2cn(-c3cc(OC)c(OC)c(OC)c3)cn2)c2ccsc2n1 ; "Pd(OH)2/C" ; "H2" ; COc1cc(-n2cnc(Nc3nc(C(C)C)nc4sccc34)c2)cc(OC)c1OC]

### Preparation of 2-isopropyl-N-(1-(3,4,5-trimethoxyphenyl)-1H-imidazol-4-yl)thieno[2,3-d]pyrimidin-4-amine (81a)

Compound 81a was prepared according to the procedure reported in step-3 of scheme 1, from 2-(prop-1-en-2-yl)-N-(1-(3,4,5-trimethoxyphenyl)-1H-imidazol-4-yl)thieno[2,3-d]pyrimidin-4-amine (80c) (500 mg, 1.18 mmol) in MeOH:DCM (60 mL, ratio: 10:1) using 50% wet, 20% Pd(OH)₂ on carbon (168 mg, 0.12 mmol) and stirring for 15 h at RT under a H₂ atmosphere. This gave after work up and purification using column chromatography [silica gel, eluting with methanolic ammonia in DCM from 2-5%] 2-isopropyl-N-(1-(3,4,5-trimethoxyphenyl)-1H-imidazol-4-yl)thieno[2,3-d]pyrimidin-4-amine (81a) (110 mg, 22% yield) as a light tan solid; ¹H NMR (300 MHz, DMSO-d₆) δ 10.52 (s, 1H, D₂O exchangeable), 8.23 (d, J=1.6 Hz, 1H), 8.17 (d, 1H), 7.98 (d, J=6.0 Hz, 1H), 7.56 (d, J=6.0 Hz, 1H), 6.93 (s, 2H), 3.87 (s, 6H), 3.69 (s, 3H), 3.20-3.04 (m, 1H), 1.37 (d, J=6.9 Hz, 6H); MS (ES+): 426.20 (M+1); Analysis calculated for: C₂₁H₂₃N₅O₃S. 0.25H₂O: C, 58.66; H, 5.51; N, 16.29; Found: C, 58.71; H, 5.40; N, 16.20.

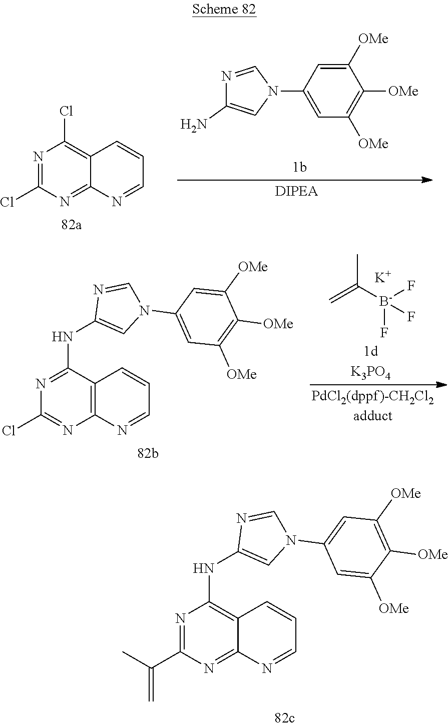

### Preparation of 2-(prop-1-en-2-yl)-N-(1-(3,4,5-trimethoxyphenyl)-1H-imidazol-4-yl)pyrido[2,3-d]pyrimidin-4-amine (82c)

### Step-1: Preparation of 2-chloro-N-(1-(3,4,5-trimethoxyphenyl)-1H-imidazol-4-yl)pyrido[2,3-d]pyrimidin-4-amine (82b)

Compound 82b was prepared according to the procedure reported in step-1 of scheme 1, from 2,4-dichloropyrido[2,3-d]pyrimidine (82a) (0.5 g, 2.49 mmol; CAS #126728-20-9) in ethanol (15 mL) using DIPEA (0.968 g, 7.497 mmol), 1-(3,4,5-trimethoxyphenyl)-1H-imidazol-4-amine (1b) (0.748 g, 3.0 mmol) and stirring at RT for 12 h. This gave after work up 2-chloro-N-(1-(3,4,5-trimethoxyphenyl)-1H-imidazol-4-yl)pyrido[2,3-d]pyrimidin-4-amine (82b) (0.66 g, 64.2% yield) as a yellow solid; ¹H NMR (300 MHz, DMSO-d) δ 11.44 (s, 1H), 9.19 (dd, J=8.3, 1.8 Hz, 1H), 9.04 (dd, J=4.4, 1.7 Hz, 1H), 8.24 (d, J=1.6 Hz, 1H), 8.01 (d, J=1.6 Hz, 1H), 7.64 (dd, J=8.3, 4.4 Hz, 1H), 6.95 (s, 2H), 3.88 (s, 6H), 3.70 (s, 3H).

### Step-2: Preparation of 2-(prop-1-en-2-yl)-N-(1-(3,4,5-trimethoxyphenyl)-1H-imidazol-4-yl)pyrido[2,3-d]pyrimidin-4-amine (82c)

Compound 82c was prepared according to the procedure reported in step-1 of scheme 1, from 2-chloro-N-(1-(3,4,5-trimethoxyphenyl)-1H-imidazol-4-yl)pyrido[2,3-d]pyrimidin-4-amine (82b) (0.66 g, 1.599 mmol) in 1,4-dioxane (19.8 mL) using potassium isopropenyltrifluoroborate (1d) (0.473 g, 3.197 mmol), potassium phosphate (0.508 g, 2.397 mmol), PdCl₂(dppf)-CH₂Cl₂ adduct (0.196 g, 0.239 mmol) and heating at 110° C. for 12 h under a nitrogen atmosphere. This gave after work up and purification using column chromatography [silica gel, eluting with MeOH in DCM from 0-2%] 2-(prop-1-en-2-yl)-N-(1-(3,4,5-trimethoxyphenyl)-1H-imidazol-4-yl)pyrido[2,3-d]pyrimidin-4-amine (82c) (0.05 g, 7.5% yield) as a reddish brown solid; ¹H NMR (300 MHz, DMSO-d₆) δ 10.97 (s, 1H), 9.16 (d, J=8.3 Hz, 1H), 9.06-8.97 (m, 1H), 8.30 (d, J=1.5 Hz, 1H), 8.22 (d, J=1.6 Hz, 1H), 7.56 (dd. J=8.2, 4.4 Hz, 1H), 6.97 (s, 2H), 6.61-6.49 (m, 1H), 5.66 (s, 1H), 3.88 (s, 6H), 3.70 (s, 3H), 2.32 (s, 3H); MS (ES+): 419.20 (M+1).

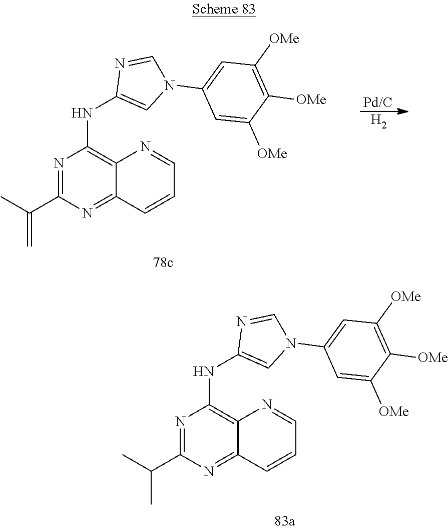 [SMILES: C=C(C)c1nc(Nc2cn(-c3cc(OC)c(OC)c(OC)c3)cn2)c2ncccc2n1 ; "Pd/C" ; "H2" ; COc1cc(-n2cnc(Nc3nc(C(C)C)nc4cccnc34)c2)cc(OC)c1OC]

### Preparation of 2-isopropyl-N-(1-(3,4,5-trimethoxyphenyl)-1H-imidazol-4-yl)pyrido[3,2-d]pyrimidin-4-amine (83a)

Compound 83 was prepared according to the procedure reported in scheme 41, from 2-(prop-1-en-2-yl)-N-(1-(3,4,5-trimethoxyphenyl)-1H-imidazol-4-yl)pyrido[3,2-d]pyrimidin-4-amine (78c) (0.14 g, 0.33 mmol) in ethanol (8.4 mL) and DCM (1.4 mL) using 50% wet, 10% Pd/C (0.142 g, 0.067 mmol) and stirring at RT for 2 h under a H₂ atmosphere. This gave after work up and purification by column chromatography [silica gel, eluting with MeOH in DCM from 0-3%] 2-isopropyl-N-(1-(3,4,5-trimethoxyphenyl)-1H-imidazol-4-yl)pyrido[3,2-d]pyrimidin-4-amine (83a) (0.048 g, 35% yield) as a yellow solid; ¹H NMR (300 MHz, DMSO-d₆) δ 9.70 (s, 1H, D₂O exchangeable), 8.86 (dd. J=4.3, 1.5 Hz, 1H), 8.27-8.09 (m, 3H), 7.88 (dd, J=8.5, 4.2 Hz, 1H), 6.96 (s, 2H), 3.88 (s, 6H), 3.70 (s, 3H), 3.24-3.11 (m, 1H), 1.39 (d, J=6.9 Hz, 6H); MS (ES+): 421.20 (M+1).

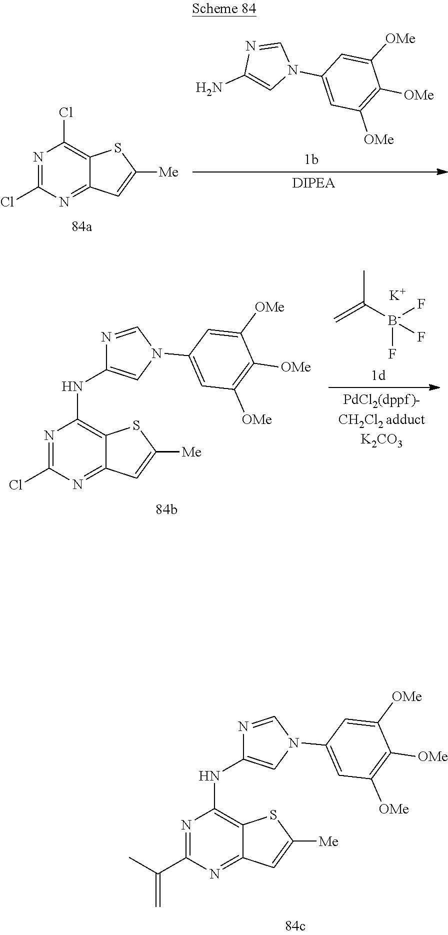

### Preparation of 6-methyl-2-(prop-1-en-2-yl)-N-(1-(3,4,5-trimethoxyphenyl)-1H-imidazol-4-yl)thieno[3,2-d]pyrimidin-4-amine (84c)

### Step-1: Preparation of 2-chloro-6-methyl-N-(1-(3,4,5-trimethoxyphenyl)-1H-imidazol-4-yl)thieno[3,2-d]pyrimidin-4-amine (84b)

Compound 84b was prepared according to the procedure reported in step-1 of scheme 1, from 2,4-dichloro-6-methylthieno[3,2-d]pyrimidine (84a) (1.0 g, 4.88 mmol; CAS #35265-82-8) 5 in EtOH (20 mL) using DIPEA (884 mg, 6.85 mmol), 1-(3,4,5-trimethoxyphenyl)-1H-imidazol-4-amine (1b) (0.568 g, 2.28 mmol) and heating at reflux for 12 h. This gave after work up 2-chloro-6-methyl-N-(1-(3,4,5-trimethoxyphenyl)-1H-imidazol-4-yl)thieno[3,2-d]pyrimidin-4-amine (84b) (450 mg, 46% yield) as a brown solid; ¹H NMR (300 MHz, DMSO-d₆) δ 8.24 (s, 2H), 7.92 (s, 1H), 7.12 (s, 1H), 6.95 (s, 2H), 3.87 (s, 6H), 3.69 (s, 3H), 2.59 (s, 3H).

### Step-2: Preparation of 6-methyl-2-(prop-1-en-2-yl)-N-(1-(3,4,5-trimethoxyphenyl)-1H-imidazol-4-yl)thieno[3,2-d]pyrimidin-4-amine (84c)

Compound 84c was prepared according to the procedure reported in step-1 of scheme 1, from 2-chloro-6-methyl-N-(1-(3,4,5-trimethoxyphenyl)-1H-imidazol-4-yl)thieno[3,2-d]pyrimidin-4-amine (84b) (300 mg, 0.695 mmol) in 1,4-dioxane (15 mL) using potassium isopropenyltrifluoroborate (1d) (0.226 g, 1.527 mmol), a solution of potassium carbonate (0.287 g, 2.07 mmol) in water (2.0 mL), PdCl₂(dppf)-CH₂Cl₂ adduct (0.113 g, 0.138 mmol) and heating at 100° C. for 12 h. This gave after work up and recrystallization using MeOH (20 mL) 6-methyl-2-(prop-1-en-2-yl)-N-(1-(3,4,5-trimethoxyphenyl)-1H-imidazol-4-yl)thieno[3,2-d]pyrimidin-4-amine (84c) (0.2 g, 66% yield) as an yellowish brown solid; ¹H NMR (300 MHz, DMSO-d₆) δ 10.33 (s, 11H, D₂O exchangeable), 8.24 (d, J=1.5 Hz, 1H), 8.09 (d, J=1.6 Hz, 1H), 7.17 (d, J=1.3 Hz, 1H), 6.94 (s, 2H), 6.45-6.32 (m, 1H), 5.48 (s, 1H), 3.87 (s, 6H), 3.69 (s, 3H), 2.66-2.58 (m, 3H), 2.28 (s, 3H); MS (ES+): 438.15 (M+1).

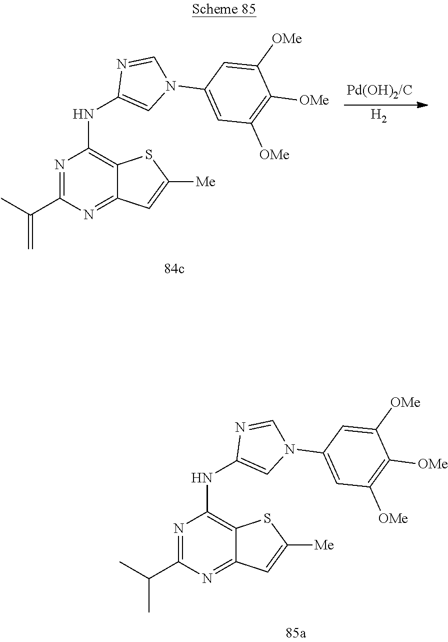 [SMILES: C=C(C)c1nc(Nc2cn(-c3cc(OC)c(OC)c(OC)c3)cn2)c2sc(C)cc2n1 ; "H2" ; COc1cc(-n2cnc(Nc3nc(C(C)C)nc4cc(C)sc34)c2)cc(OC)c1OC]

### Preparation of 2-isopropyl-6-methyl-N-(1-(3,4,5-trimethoxyphenyl)-1H-imidazol-4-yl)thieno[3,2-d]pyrimidin-4-amine (85a)

Compound 85a was prepared according to the procedure reported in step-3 of scheme 1, from 6-methyl-2-(prop-1-en-2-yl)-N-(1-(3,4,5-trimethoxyphenyl)-1H-imidazol-4-yl)thieno[3,2-d]pyrimidin-4-amine (84c) (0.15 g, 0.343 mmol) in MeOH:DCM (ratio 10:2, 20 mL) using 50% wet, 20% Pd(OH)₂ on carbon (7 mg, 0.005 mmol) and stirring at RT for 12 h under a H₂ atmosphere. This gave after work up and recrystallization using MeOH (10 mL) 2-isopropyl-6-methyl-N-(1-(3,4,5-trimethoxyphenyl)-1H-imidazol-4-yl)thieno[3,2-d]pyrimidin-4-amine (85a) (0.055 g, 36% yield) as an grayish brown solid; ¹H NMR (300 MHz, DMSO-d₆) δ 10.22 (s, 1H, D₂O exchangeable), 8.21 (d, J=1.6 Hz, 1H), 8.11 (d, J=1.6 Hz, 1H), 7.09 (d, J=1.3 Hz, 1H), 6.93 (s, 2H), 3.87 (s, 6H), 3.69 (s, 3H), 3.14-2.98 (m, 1H), 2.58 (s, 3H), 1.35 (d, J=6.9 Hz, 6H); MS (ES+): 440.20 (M+1).

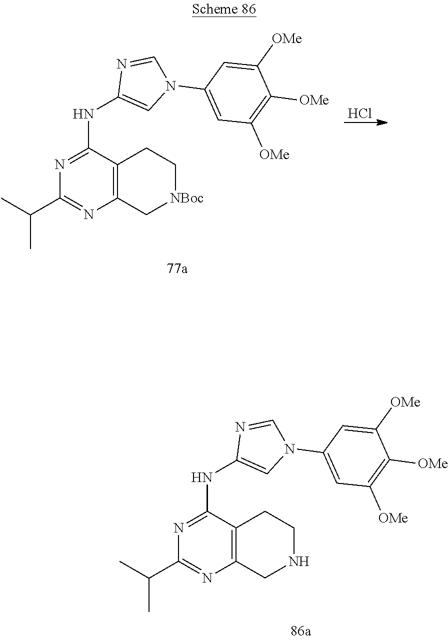 [SMILES: COc1cc(-n2cnc(Nc3nc(C(C)C)nc4c3CCN(C(=O)OC(C)(C)C)C4)c2)cc(OC)c1OC ; Cl ; COc1cc(-n2cnc(Nc3nc(C(C)C)nc4c3CCNC4)c2)cc(OC)c1OC]

### Preparation of 2-isopropyl-N-(1-(3,4,5-trimethoxyphenyl)-1H-imidazol-4-yl)-5,6,7,8-tetrahydropyrido[3,4-d]pyrimidin-4-amine (86a)

To a stirred solution of tert-butyl 2-isopropyl-4-((1-(3,4,5-trimethoxyphenyl)-1H-imidazol-4-yl)amino)-5,6-dihydropyrido[3,4-d]pyrimidine-7 (8H)-carboxylate (77a) (0.17 g, 0.32 mmol) in EtOH (3 mL) was added 23% HCl in EtOH (1 mL) and heated at 70° C. for 2 h. The reaction mixture was concentrated and the residue obtained was triturated with DCM:Ethyl acetate (ratio 1:1, 5.0 mL) and filtered to give 2-isopropyl-N-(1-(3,4,5-trimethoxyphenyl)-1H-imidazol-4-yl)-5,6,7,8-tetrahydropyrido[3,4-d]pyrimidin-4-amine (86a) (0.145 g, 98% yield) HCl salt as an light brown solid; ¹H NMR (300 MHz, DMSO-d₆) δ 9.86 (s, 3H, D₂O exchangeable), 8.48 (s, 1H), 8.11 (d, J=1.6 Hz, 1H), 6.97 (s, 2H), 4.33 (s, 2H), 3.87 (s, 6H), 3.69 (s, 3H), 3.54-3.37 (m, 2H), 3.30-3.09 (m, 1H), 2.94 (s, 2H), 1.36 (d, J=6.8 Hz, 6H); MS (ES+): 425.20 (M+1).

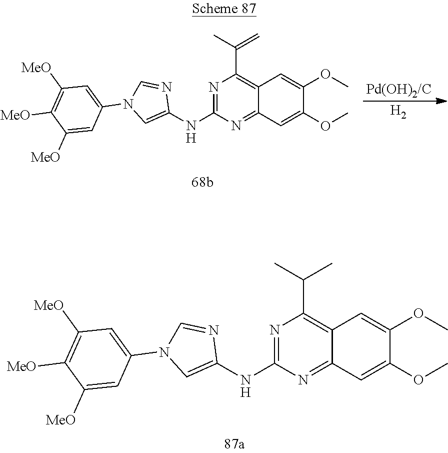

### Preparation of 4-isopropyl-6,7-dimethoxy-N-(1-(3,4,5-trimethoxyphenyl)-1H-imidazol-4-yl)quinazolin-2-amine (87a)

Compound 87a was prepared according to the procedure reported in step-3 of scheme 1, from 6,7-dimethoxy-4-(prop-1-en-2-yl)-N-(1-(3,4,5-trimethoxyphenyl)-1H-imidazol-4-yl)quinazolin-2-amine (68b) (0.20 g, 0.419 mmol) in MeOH (24 mL) and DCM (2.4 mL) using 50% wet, 20% Pd(OH)₂ on carbon (0.044 g, 0.0313 mmol) and stirring for 16 h at RT under a H₂ atmosphere. This gave after work up and recrystallization using diethyl ether (5.0 mL) 4-isopropyl-6,7-dimethoxy-N-(1-(3,4,5-trimethoxyphenyl)-1H-imidazol-4-yl)quinazolin-2-amine (87a) (70 mg, 35% yield) as an grayish brown solid; ¹H NMR (300 MHz, DMSO-d₆) δ 9.36 (s, 1H, D₂O exchangeable), 8.07 (s, 1H), 8.01 (s, 1H), 7.34 (s, 1H), 7.14 (s, 1H), 6.92 (s, 2H), 3.92 (s, 3H), 3.91-3.80 (m, 10H), 3.69 (s, 3H), 1.36 (d, J=6.6 Hz, 6H); MS (ES+): 480.20 (M+1).

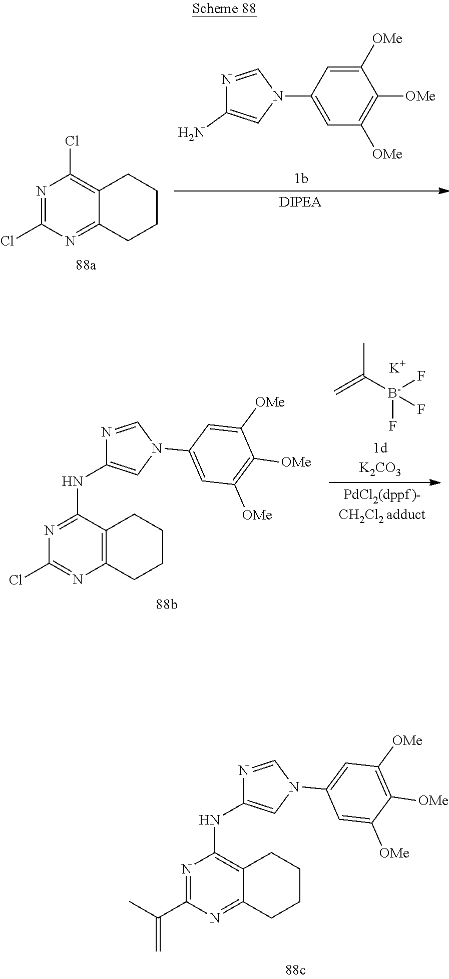

### Preparation of 2-(prop-1-en-2-yl)-N-(1-(3,4,5-trimethoxyphenyl)-1H-imidazol-4-yl)-5,6,7,8-tetrahydroquinazolin-4-amine (88c)

### Step-1: Preparation of 2-chloro-N-(1-(3,4,5-trimethoxyphenyl)-1H-imidazol-4-yl)-5,6,7,8-tetrahydroquinazolin-4-amine (88b)

Compound 88b was prepared according to the procedure reported in step-1 of scheme 1, from 2,4-dichloro-5,6,7,8-tetrahydroquinazoline (88a) (0.8 g, 3.94 mmol; CAS #1127-85-1) in EtOH (16 mL) using DIPEA (1.52 g, 11.81 mmol), 1-(3,4,5-trimethoxyphenyl)-1H-imidazol-4-amine (1b) (1.17 g, 4.69 mmol) and heating at 78° C. for 16 h. This gave after work up 2-chloro-N-(1-(3,4,5-trimethoxyphenyl)-1H-imidazol-4-yl)-5,6,7,8-tetrahydroquinazolin-4-amine (88b) (0.40 g, 25% yield) as a brown solid; ¹H NMR (300 MHz, DMSO-d₆) δ 9.29 (s, 1H), 8.15 (d, J=1.7 Hz, 1H), 7.77 (d, J=1.6 Hz, 1H), 6.90 (s, 2H), 3.87 (s, 6H), 3.69 (s, 3H), 2.67-2.53 (m, 4H), 1.76 (m, 4H).

### Step-2: Preparation of 2-(prop-1-en-2-yl)-N-(1-(3,4,5-trimethoxyphenyl)-1H-imidazol-4-yl)-5,6,7,8-tetrahydroquinazolin-4-amine (88c)

Compound 88c was prepared according to the procedure reported in step-1 of scheme 1, from 2-chloro-N-(1-(3,4,5-trimethoxyphenyl)-1H-imidazol-4-yl)-5,6,7,8-tetrahydroquinazolin-4-amine (88b) (0.4 g, 0.962 mmol) in 1,4-dioxane (12 mL) using potassium isopropenyltrifluoroborate (l d) (0.427 g, 2.88 mmol), a solution of K₂CO₃ (0.398 g, 2.88 mmol) in H₂O (1.2 mL), PdCl₂(dppf)-CH₂Cl₂ adduct (0.157 g, 0.192 mmol) and heating at 110° C. for 16 h under a nitrogen atmosphere. This gave after work up and purification using column chromatography [silica gel, eluting with MeOH in DCM from 0-5%] 2-(prop-1-en-2-yl)-N-(1-(3,4,5-trimethoxyphenyl)-1H-imidazol-4-yl)-5,6,7,8-tetrahydroquinazolin-4-amine (88c) (0.27 g, 66% yield) as an greenish yellow solid; ¹H NMR (300 MHz, DMSO-d₆) δ 8.89 (s, 1H), 8.20 (d, J=1.6 Hz, 1H), 8.03 (d, J=1.6 Hz, 1H), 6.91 (s, 2H), 6.29 (d, J=2.8 Hz, 1H), 5.42 (s, 1H), 3.87 (s, 6H), 3.69 (s, 3H), 2.74-2.56 (m, 4H), 2.23 (s, 3H), 1.88-1.67 (m, 4H); MS (ES+): 422.20 (M+1).

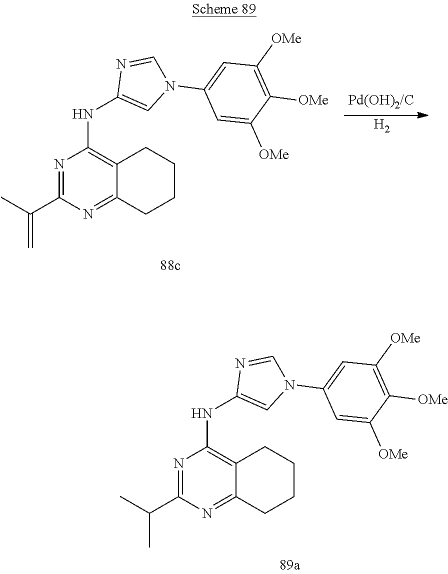 [SMILES: C=C(C)c1nc2c(c(Nc3cn(-c4cc(OC)c(OC)c(OC)c4)cn3)n1)CCCC2 ; "H2" ; COc1cc(-n2cnc(Nc3nc(C(C)C)nc4c3CCCC4)c2)cc(OC)c1OC]

### Preparation of 2-isopropyl-N-(1-(3,4,5-trimethoxyphenyl)-1H-imidazol-4-yl)-5,6,7,8-tetrahydroquinazolin-4-amine (89a)

Compound 89a was prepared according to the procedure reported in step-3 of scheme 1, from 2-(prop-1-en-2-yl)-N-(1-(3,4,5-trimethoxyphenyl)-1H-imidazol-4-yl)-5,6,7,8-tetrahydroquinazolin-4-amine (88c) (0.15 g, 0.356 mmol) in MeOH (30 mL) and DCM (3 mL) using 50% wet, 20% Pd(OH)₂ on carbon (0.037 g 0.0263 mmol) and stirring for 16 h at RT under a H₂ atmosphere. This gave after work up and recrystallization using diethyl ether (5 mL) 2-isopropyl-N-(1-(3,4,5-trimethoxyphenyl)-1H-imidazol-4-yl)-5,6,7,8-tetrahydroquinazolin-4-amine (89a) (0.14 g, 93% yield) as a gray solid; ¹H NMR (300 MHz, DMSO-d₆) δ 8.22 (s, 1H), 8.06 (d, J=1.6 Hz, 1H), 6.90 (s, 2H), 3.86 (s, 6H), 3.69 (s, 3H), 3.13-2.93 (m, 1H), 2.76-2.54 (m, 4H), 1.90-1.64 (m, 4H), 1.32 (d, J=6.8 Hz, 6H); MS (ES+): 424.30 (M+1).

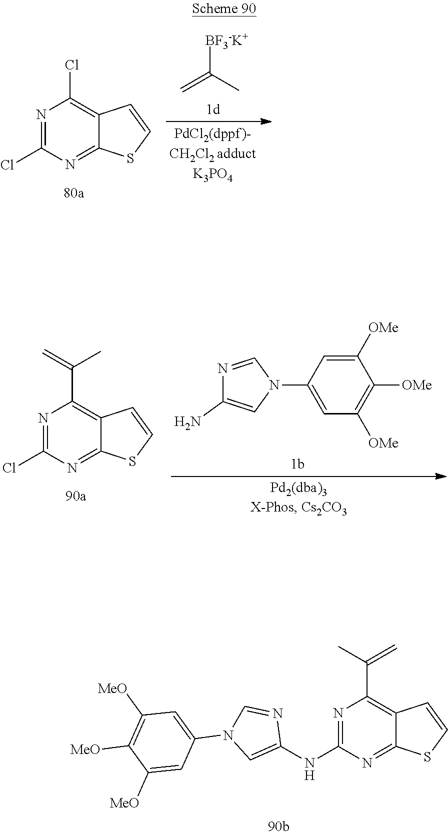

### Preparation of 4-(prop-1-en-2-yl)-N-(1-(3,4,5-trimethoxyphenyl)-1H-imidazol-4-yl)thieno[2,3-d]pyrimidin-2-amine (90b)

### Step-1: Preparation of 2-chloro-4-(prop-1-en-2-yl)thieno[2,3-d]pyrimidine (90a)

Compound 90a was prepared according to the procedure reported in step-1 of scheme 1, from 2,4-dichlorothieno[2,3-d]pyrimidine (80a) (2 g 9.75 mmol) in toluene (100 mL) using potassium isopropenyltrifluoroborate (1d) (1.44 g, 9.73 mmol), a solution of potassium phosphate (3.1 g, 14.62 mmol) in water (2 mL), PdCl₂(dppf)-CH₂Cl₂ adduct (0.8 g, 0.975 mmol) and heating at 50° C. for 3 h. This gave after work up and purification using column chromatography [silica gel, eluting with EtOAc in n-heptane from 0-10%] 2-chloro-4-(prop-1-en-2-yl)thieno[2,3-d]pyrimidine (90a) (1.5 g, 7.12 mmol) as a white solid; ¹H NMR (300 MHz, DMSO-d₆) δ 8.02 (d, J=6.1 Hz, 1H), 7.71 (d, J=6.1 Hz, 1H), 5.86 (d, J=8.7 Hz, 2H), 2.24 (s, 3H).

### Step-2: Preparation of 4-(prop-1-en-2-yl)-N-(1-(3,4,5-trimethoxyphenyl)-1H-imidazol-4-yl)thieno[2,3-d]pyrimidin-2-amine (90b)

Compound 90b was prepared according to the procedure reported in step-4 of scheme 27, from 2-chloro-4-(prop-1-en-2-yl)thieno[2,3-d]pyrimidine (90a) (500 mg, 2.37 mmol) in 1,4-dioxane (50 mL) using 1-(3,4,5-trimethoxyphenyl)-1H-imidazol-4-amine (1b) (720 mg, 3.08 mmol), Pd₂(dba)₃ (217 mg, 0.237 mmol), X-phos (0.452 g, 0.95 mmol), Cs₂CO₃ (3.10 g, 9.48 mmol) and heating at 100° C. for 15 h. This gave after work up and purification using column chromatography [silica gel, eluting with 5% methanolic ammonia in DCM]4-(prop-1-en-2-yl)-N-(1-(3,4,5-trimethoxyphenyl)-1H-imidazol-4-yl)thieno[2,3-d]pyrimidin-2-amine (90b) (260 mg, 26% yield) as a fluorescent green solid; ¹H NMR (300 MHz, DMSO-d₆) δ 9.93 (s, 1H, D₂O exchangeable), 8.11 (d, J=1.6 Hz, 1H), 7.83 (d, J=1.6 Hz, 1H), 7.45 (s, 2H), 6.91 (s, 2H), 5.77 (d, J=16.3 Hz, 2H), 3.87 (s, 6H), 3.69 (s, 3H), 2.30 (s, 3H); MS (ES+): 424.20 (M+1); Analysis calculated for: C₂₁H₂₁N₅O₃S: C, 59.56; H, 5.00; N, 16.54; Found: C, 59.39; H, 4.96; N, 16.32.

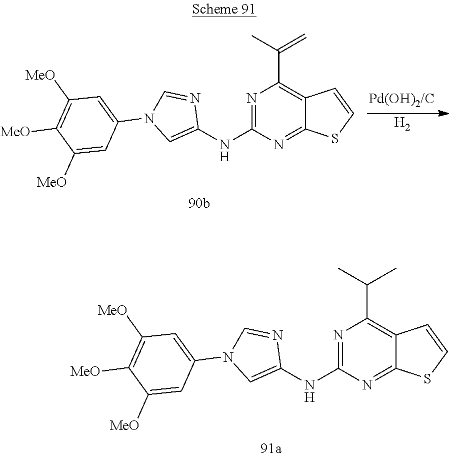 [SMILES: C=C(C)c1nc(Nc2cn(-c3cc(OC)c(OC)c(OC)c3)cn2)nc2sccc12 ; "H2" ; COc1cc(-n2cnc(Nc3nc(C(C)C)c4ccsc4n3)c2)cc(OC)c1OC]

### Preparation of 4-isopropyl-N-(1-(3,4,5-trimethoxyphenyl)-1H-imidazol-4-yl)thieno[2,3-d]pyrimidin-2-amine (91a)

Compound 91a was prepared according to the procedure reported in step-3 of scheme 1, from 4-(prop-1-en-2-yl)-N-(1-(3,4,5-trimethoxyphenyl)-1H-imidazol-4-yl)thieno[2,3-d]pyrimidin-2-amine (90b) (150 mg, 0.354 mmol) in MeOH:DCM:CH₃COOH (50 mL, ratio: 50:1:0.2) using 50% wet, 20% Pd(OH)? on carbon (100 mg, 0.07 mmol) and stirring for 15 h at RT under a H₂ atmosphere. This gave after work up and purification using column chromatography [silica gel, eluting with methanolic ammonia in DCM from 2-5%] 4-isopropyl-N-(1-(3,4,5-trimethoxyphenyl)-1H-imidazol-4-yl)thieno[2,3-d]pyrimidin-2-amine (91a) (90 mg, 60% yield) as a off-white solid; ¹H NMR (300 MHz, DMSO-d₆) δ 9.86 (s, 1H, D₂O exchangeable), 8.12 (d, J=1.6 Hz, 1H), 7.90 (s, 1H), 7.49 (d, J=6.0 Hz, 1H), 7.41 (d, J=6.0 Hz, 1H), 6.92 (s, 2H), 3.88 (s, 6H), 3.69 (s, 3H), 3.64-3.49 (m, 1H), 1.37 (d, J=6.8 Hz, 6H); MS (ES+): 426.20 (M+1); Analysis calculated for: C₂₁H₂₃N₅O₃S. 0.25 H₂O: C, 58.66; H, 5.51; N, 16.29; Found: C, 58.78; H, 5.56; N, 16.42.

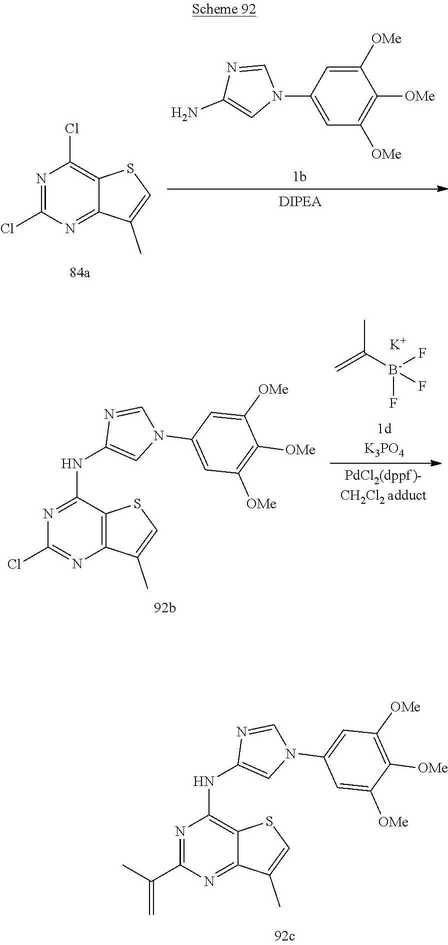

### Preparation of 7-methyl-2-(prop-1-en-2-yl)-N-(1-(3,4,5-trimethoxyphenyl)-1H-imidazol-4-yl)thieno[3,2-d]pyrimidin-4-amine (92c)

### Step-1: Preparation of 2-chloro-7-methyl-N-(1-(3,4,5-trimethoxyphenyl)-1H-imidazol-4-yl)thieno[3,2-d]pyrimidin-4-amine (92b)

Compound 92b was prepared according to the procedure reported in step-1 of scheme 1, from 2,4-dichloro-7-methylthieno[3,2-d]pyrimidine (92a) (0.7 g, 3.195 mmol; CAS #35265-83-9) in IPA (21 mL) using DIPEA (1.238 g, 9.585 mmol), 1-(3,4,5-trimethoxyphenyl)-1H-imidazol-4-amine (1b) (0.955 g, 3.834 mmol) and heating at reflux for 12 h. This gave after work up 2-chloro-7-methyl-N-(1-(3,4,5-trimethoxyphenyl)-1H-imidazol-4-yl)thieno[3,2-d]pyrimidin-4-amine (92b) (0.5 g, 36% yield) as an off white solid; ¹H NMR (300 MHz, Chloroform-d) δ 7.96 (s, 11H), 7.84 (s, 1H), 7.59 (s, 1H), 7.37 (d, J=1.3 Hz, 1H), 6.61 (s, 2H), 3.88 (s, 6H), 3.83 (s, 3H), 2.40 (d, J=1.2 Hz, 3H).

### Step-2: Preparation of 7-methyl-2-(prop-1-en-2-yl)-N-(1-(3,4,5-trimethoxyphenyl)-1H-imidazol-4-yl)thieno[3,2-d]pyrimidin-4-amine (92c)

Compound 92c was prepared according to the procedure reported in step-1 of scheme 1, from 2-chloro-7-methyl-N-(1-(3,4,5-trimethoxyphenyl)-1H-imidazol-4-yl)thieno[3,2-d]pyrimidin-4-amine (92b) (0.4 g, 0.926 mmol) in 1,4-dioxane (12 mL) using potassium isopropenyltrifluoroborate (id) (0.358 g, 2.42 mmol), a solution of potassium phosphate (0.411 g, 1.936 mmol) in water (1 mL), PdCl₂(dppf)-CH₂Cl₂ adduct (0.118 g, 0.145 mmol) and heating at reflux for 12 h under a nitrogen atmosphere. This gave after work up and purification using column chromatography [silica gel, eluting with MeOH in DCM from 0-3%)] 7-methyl-2-(prop-1-en-2-yl)-N-(1-(3,4,5-trimethoxyphenyl)-1H-imidazol-4-yl)thieno[3,2-d]pyrimidin-4-amine (92c) (0.2 g, 50% yield) as a white solid; ¹H NMR (300 MHz, DMSO-d₆) δ 10.45 (s, 1H, D₂O exchangeable), 8.25 (d, J=1.6 Hz, 1H), 8.14 (d, J=1.6 Hz, 1H), 7.81 (d, J=1.4 Hz, 1H), 6.96 (s, 2H), 6.47 (d, 1H), 5.52 (s, 1H), 3.88 (s, 6H), 3.70 (s, 3H), 2.39 (d, J=1.2 Hz, 3H), 2.33 (s, 3H); MS (ES+): 438.10 (M+1); (ES−): 436.10 (M−1).

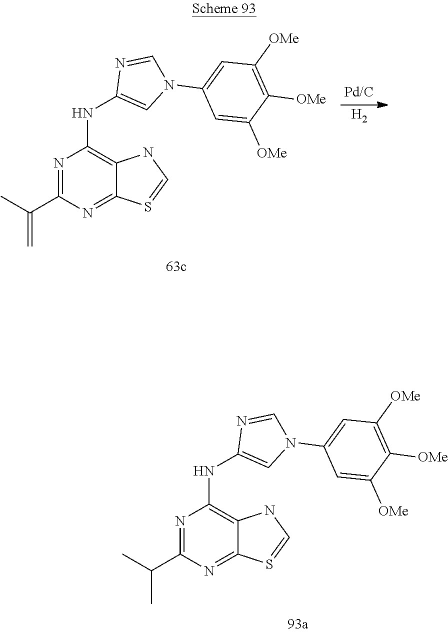

### Preparation of 5-isopropyl-N-(1-(3,4,5-trimethoxyphenyl)-1H-imidazol-4-yl)thiazolo[5,4-d]pyrimidin-7-amine (93a)

Compound 93a was prepared according to the procedure reported in scheme 41, from 5-(prop-1-en-2-yl)-N-(1-(3,4,5-trimethoxyphenyl)-1H-imidazol-4-yl)thiazolo[5,4-d]pyrimidin-7-amine (63c) (50 mg, 0.118 mmol) in methanol (20 mL) using Pd/C (37.6 mg, 0.035 mmol) and stirring at RT for 5 h under a H₂ atmosphere. This gave after work up and purification using reverse phase column chromatography [C18 column (30 g), eluting with ACN in water (containing 0.1% HCl) from 0-100%] 5-isopropyl-N-(1-(3,4,5-trimethoxyphenyl)-1H-imidazol-4-yl)thiazolo[5,4-d]pyrimidin-7-amine (93a) (11 mg, 22% yield) HCl salt as an off-white solid; ¹H NMR (300 MHz, DMSO-d₆) δ 10.63 (s, 1H, D₂O exchangeable), 9.35 (s, 1H), 8.69 (s, 1H, D₂O exchangeable), 8.20 (s, 1H, D₂O exchangeable), 7.03 (s, 2H), 3.88 (s, 6H), 3.71 (s, 3H), 3.26-3.08 (m, 1H), 1.35 (d, J=6.6 Hz, 6H); MS (ES+): 427.1 (M+1).

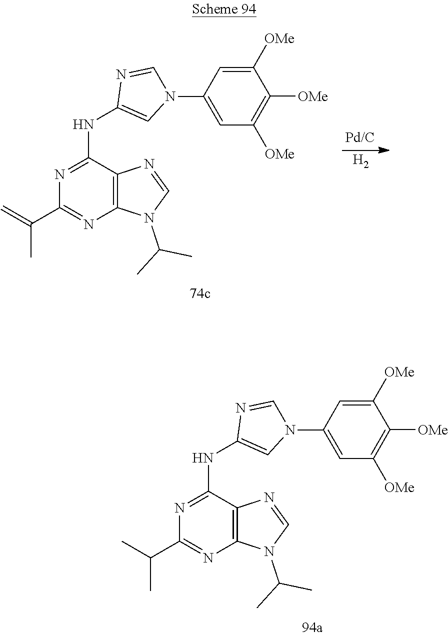 [SMILES: C=C(C)c1nc(Nc2cn(-c3cc(OC)c(OC)c(OC)c3)cn2)c2ncn(C(C)C)c2n1 ; "H2" ; COc1cc(-n2cnc(Nc3nc(C(C)C)nc4c3ncn4C(C)C)c2)cc(OC)c1OC]

### Preparation of 2,9-diisopropyl-N-(1-(3,4,5-trimethoxyphenyl)-1H-imidazol-4-yl)-9H-purin-6-amine (94a)

Compound 94a was prepared according to the procedure reported in scheme 41, from 9-isopropyl-2-(prop-1-en-2-yl)-N-(1-(3,4,5-trimethoxyphenyl)-1H-imidazol-4-yl)-9H-purin-6-amine (74c) (60 mg, 0.133 mmol) in methanol (20 mL) using Pd/C (42.6 mg, 0.040 mmol) and stirring at RT for 5 h under a H₂ atmosphere. This gave after work up and purification using reverse phase column chromatography [C18 column (30 g), eluting with ACN in water (containing 0.1% HCl) from 0-100%] 2,9-diisopropyl-N-(1-(3,4,5-trimethoxyphenyl)-1H-imidazol-4-yl)-9H-purin-6-amine (94a) (20 mg, 33% yield) HCl salt as an off-white solid; ¹H NMR (300 MHz, DMSO-d₆) δ 11.21 (s, 1H, D₂O exchangeable), 8.77 (s, 1H), 8.70 (s, 1H), 8.03 (d, J=1.7 Hz, 1H), 7.04 (s, 2H), 4.93-4.80 (m, 1H), 3.88 (s, 6H), 3.71 (s, 3H), 3.26-3.12 (m, 1H), 1.58 (d, J=6.8 Hz, 6H), 1.38 (d, J=6.8 Hz, 6H); MS (ES+): 452.3 (M+1).

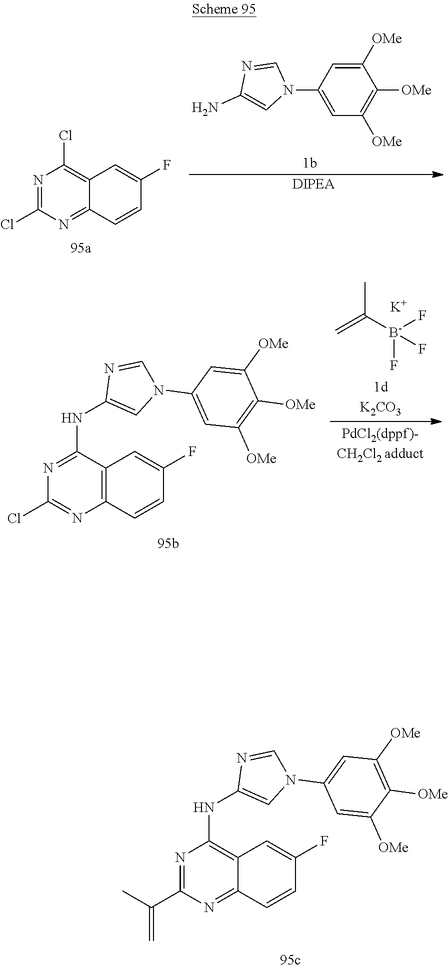

### Preparation of 6-fluoro-2-(prop-1-en-2-yl)-N-(1-(3,4,5-trimethoxyphenyl)-1H-imidazol-4-yl)quinazolin-4-amine (95c)

### Step-1: Preparation of 2-chloro-6-fluoro-N-(1-(3,4,5-trimethoxyphenyl)-1H-imidazol-4-yl)quinazolin-4-amine (95b)

Compound 95b was prepared according to the procedure reported in step-1 of scheme 1, from 2,4-dichloro-6-fluoroquinazoline (95a) (1.0 g, 4.61 mmol; CAS #134517-57-0) in EtOH (20 mL) using DIPEA (2.084 mg, 16.123 mmol), 1-(3,4,5-trimethoxyphenyl)-1H-imidazol-4-amine (1b) (1.57 g, 5.49 mmol) and stirring at RT for 12 h. This gave after work up 2-chloro-6-fluoro-N-(1-(3,4,5-trimethoxyphenyl)-1H-imidazol-4-yl)quinazolin-4-amine (95b) (1.0 g, 51% yield) as a brown solid; ¹H NMR (300 MHz, DMSO-d₆) δ 11.14 (s, 1H), 8.65 (d, J=10.1 Hz, 1H), 8.23 (d, J=1.5 Hz, 1H), 8.00 (d, J=1.5 Hz, 1H), 7.80 (d, J=6.3 Hz, 2H), 6.94 (s, 2H), 3.88 (s, 6H), 3.70 (s, 3H).

### Step-2: Preparation of 6-fluoro-2-(prop-1-en-2-yl)-N-(1-(3,4,5-trimethoxyphenyl)-1H-imidazol-4-yl)quinazolin-4-amine (95c)

Compound 95c was prepared according to the procedure reported in step-1 of scheme 1, 2-chloro-6-fluoro-N-(1-(3,4,5-trimethoxyphenyl)-1H-imidazol-4-yl)quinazolin-4-amine (95b) (1.0 g, 2.326 mmol) in 1,4-dioxane (20 mL) using potassium isopropenyltrifluoroborate (1 d) (1.03 g, 6.960 mmol), a solution of potassium carbonate (0.964 g, 6.975 mmol) in water (2 mL), PdCl₂(dppf)-CH₂Cl₂ adduct (0.569 g, 0.696 mmol) and heating at 140° C. for 12 h. This gave after work up and recrystallization using MeOH (20 mL) 6-fluoro-2-(prop-1-en-2-yl)-N-(1-(3,4,5-trimethoxyphenyl)-1H-imidazol-4-yl)quinazolin-4-amine (95c) (0.280 g, 28% yield) as an off white solid; ¹H NMR (300 MHz, DMSO-d₆) δ 10.68 (s, 1H), 8.63 (d, J=10.1 Hz, 1H), 8.30 (s, 1H), 8.22 (s, 1H), 7.95-7.81 (m, 1H), 7.81-7.62 (m, 1H), 6.97 (s, 2H), 6.50 (s, 1H), 5.60 (s, 1H), 3.89 (s, 6H), 3.70 (s, 3H), 2.31 (s, 3H) ¹⁹F NMR (282 MHz, DMSO) δ −112.91; MS (ES+): 436.20 (M+1); (ES−): 434.10 (M−1).

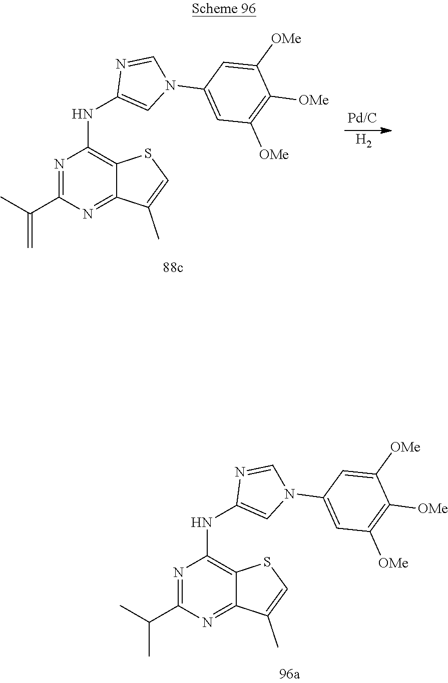 [SMILES: C=C(C)c1nc(Nc2cn(-c3cc(OC)c(OC)c(OC)c3)cn2)c2scc(C)c2n1 ; "H2" ; COc1cc(-n2cnc(Nc3nc(C(C)C)nc4c(C)csc34)c2)cc(OC)c1OC]

### Preparation of 2-isopropyl-7-methyl-N-(1-(3,4,5-trimethoxyphenyl)-1H-imidazol-4-yl)thieno[3,2-d]pyrimidin-4-amine (96a)

Compound 96a was prepared according to the procedure reported in scheme 41, from 7-methyl-2-(prop-1-en-2-yl)-N-(1-(3,4,5-trimethoxyphenyl)-1H-imidazol-4-yl)thieno[3,2-d]pyrimidin-4-amine (92c) (0.14 g, 0.32 mmol) in ethanol (4.2 mL) and DCM (2.1 mL) using 50% wet, 10% Pd/C (0.136 g, 0.064 mmol) and stirring at RT for 12 h under a H₂ atmosphere. This gave after work up and purification using column chromatography (silica gel, eluting with MeOH in DCM from 0-3%) 2-isopropyl-7-methyl-N-(1-(3,4,5-trimethoxyphenyl)-1H-imidazol-4-yl)thieno[3,2-d]pyrimidin-4-amine (96a) (0.024 g, 17% yield) as a white solid; ¹H NMR (300 MHz, DMSO-d₆) δ 10.35 (s, 1H), 8.22 (d, J=1.6 Hz, 1H), 8.15 (d, J=1.6 Hz, 1H), 7.75 (d, J=1.4 Hz, 1H), 6.94 (d, J=4.7 Hz, 2H), 3.87 (s, 6H), 3.69 (s, 3H), 3.21-3.05 (m, 1H), 2.34 (s, 3H), 1.38 (d, J=6.9 Hz, 6H); MS (ES+): 440.10 (M+1); Analysis calculated for: C₂₂H₂₅N₅O₃S·0.5H₂O: C, 58.91; H, 5.84; N, 15.61; Found: C, 59.08; H, 5.50; N, 15.79.

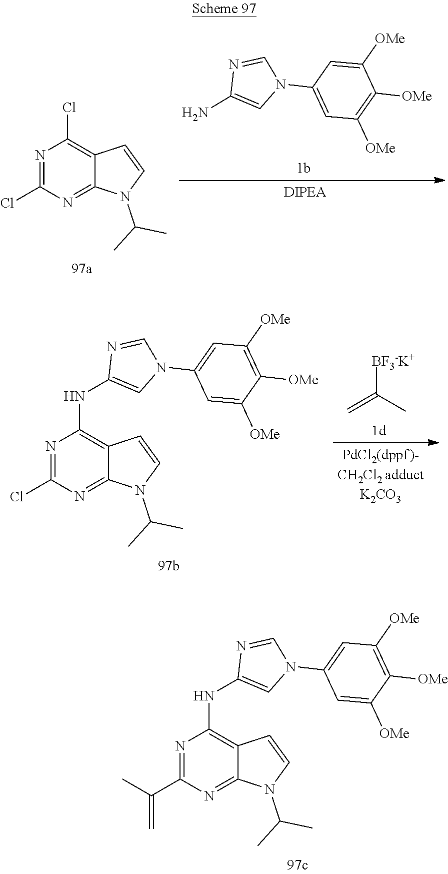

### Preparation of 7-isopropyl-2-(prop-1-en-2-yl)-N-(1-(3,4,5-trimethoxyphenyl)-1H-imidazol-4-yl)-7H-pyrrolo[2,3-d]pyrimidin-4-amine (97c)

### Step-1: Preparation of 2-chloro-7-isopropyl-N-(1-(3,4,5-trimethoxyphenyl)-1H-imidazol-4-yl)-7H-pyrrolo[2,3-d]pyrimidin-4-amine (97b)

Compound 97b was prepared according to the procedure reported in step-1 of scheme 1, from 2,4-dichloro-7-isopropyl-7H-pyrrolo[2,3-d]pyrimidine (97a) (1.5 g, 6.52 mmol; CAS #1227635-12-2) in IPA (30 mL) using DIPEA (2.94 g, 22.81 mmol), 1-(3,4,5-trimethoxyphenyl)-1H-imidazol-4-amine (1b) (01.95 g, 7.82 mmol) and heating at reflux for 24 h. This gave after work up 2-chloro-7-isopropyl-N-(1-(3,4,5-trimethoxyphenyl)-1H-imidazol-4-yl)-7H-pyrrolo[2,3-d]pyrimidin-4-amine (97b) (0.400 g, 14%) as a brown solid; ¹H NMR (300 MHz, DMSO-d₆) δ 10.47 (s, 1H), 8.15 (d, J=1.6 Hz, 1H), 7.85 (d, 0.1=1.6 Hz, 1H), 7.38 (d, J=3.6 Hz, 1H), 6.91 (s, 3H), 4.99-4.75 (m, 1H), 3.88 (s, 6H), 3.70 (s, 3H), 1.43 (d, J=6.7 Hz, 6H).

### Step-2: Preparation of 7-isopropyl-2-(prop-1-en-2-yl)-N-(1-(3,4,5-trimethoxyphenyl)-1H-imidazol-4-yl)-7H-pyrrolo[2,3-d]pyrimidin-4-amine (97c)

Compound 97c was prepared according to the procedure reported in step-1 of scheme 1, from 2-chloro-7-isopropyl-N-(1-(3,4,5-trimethoxyphenyl)-1H-imidazol-4-yl)-7H-pyrrolo[2,3-d]pyrimidin-4-amine (97b) (0.4 g, 0.9 mmol) in 1,4-dioxane (20 mL) using potassium isopropenyltrifluoroborate (1d) (0.334 g, 2.25 mmol), a solution of potassium carbonate (0.37 g, 2.7 mmol) in water (4 mL), PdCl₂(dppf)-CH₂Cl₂ adduct (0.164 g, 0.18 mmol) and heating at 110° C. for 16 h. This gave after work up and purification using column chromatography [silica gel, eluting with MeOH in DCM from 0-5%] 7-isopropyl-2-(prop-1-en-2-yl)-N-(1-(3,4,5-trimethoxyphenyl)-1H-imidazol-4-yl)-7H-pyrrolo[2,3-d]pyrimidin-4-amine (97c) (0.25 g, 61% yield) as an light tan solid; ¹H NMR (300 MHz, DMSO-d₆) δ 10.15 (s, 1H), 8.21 (d, J=1.6 Hz, 1H), 8.11 (d, J=1.7 Hz, 1H), 7.37 (d, J=3.6 Hz, 1H), 7.03-6.87 (m, 3H), 6.36 (d, J=2.9 Hz, 1H), 5.42 (s, 1H), 5.06-4.87 (m, 1H), 3.87 (s, 6H), 3.69 (s, 3H), 2.30 (s, 3H), 1.46 (d, J=6.8 Hz, 6H); MS (ES+): 449.20 (M+1); Analysis calculated for: C₂₄H₂₈N₆O₃·0.25H₂O; C, 63.63; H, 6.34; N, 18.55; Found: C, 63.54; H, 6.41; N, 18.19.

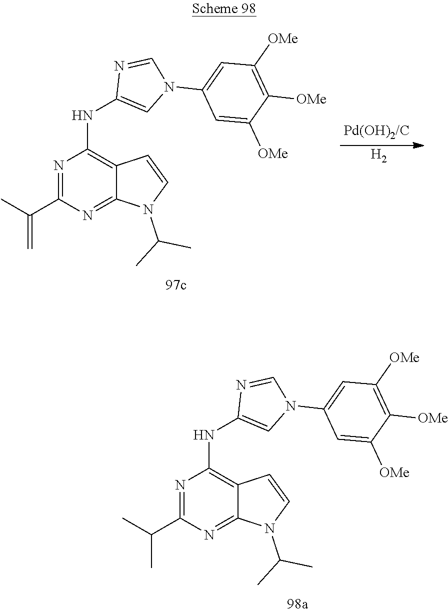 [SMILES: C=C(C)c1nc(Nc2cn(-c3cc(OC)c(OC)c(OC)c3)cn2)c2ccn(C(C)C)c2n1 ; "H2" ; COc1cc(-n2cnc(Nc3nc(C(C)C)nc4c3ccn4C(C)C)c2)cc(OC)c1OC]

### Preparation of 2,7-diisopropyl-N-(1-(3,4,5-trimethoxyphenyl)-1H-imidazol-4-yl)-7H-pyrrolo[2,3-d]pyrimidin-4-amine (98a)

Compound 98a was prepared according to the procedure reported in scheme 66, from 7-isopropyl-2-(prop-1-en-2-yl)-N-(1-(3,4,5-trimethoxyphenyl)-1H-imidazol-4-yl)-7H-pyrrolo[2,3-d]pyrimidin-4-amine (97c) (0.15 g, 0.33 mmol) in MeOH (9.0 mL) and DCM (0.9 mL) using 50% wet, 20% Pd(OH)₂ on carbon (0.093 g 0.066 mmol) and stirring at RT for 16 h under a hydrogen atmosphere. This gave after work up and purification using column chromatography [silica gel, eluting with MeOH in DCM from 0-5%] 2,7-diisopropyl-N-(1-(3,4,5-trimethoxyphenyl)-1H-imidazol-4-yl)-7H-pyrrolo[2,3-d]pyrimidin-4-amine (98a) (75 mg, 51% yield) as a brown solid; ¹H NMR (300 MHz, DMSO₆) δ 10.03 (s, 1H), 8.27-8.09 (m, 2H), 7.27 (d, J=3.6 Hz, 1H), 6.97-6.83 (m, 3H), 5.02-4.82 (m, 1H), 3.87 (s, 6H), 3.69 (s, 3H), 3.14-2.99 (m, 1H), 1.43 (d, J=6.8 Hz, 6H), 1.37 (d, J=6.8 Hz, 6H); MS (ES+): 451.20 (M+1).

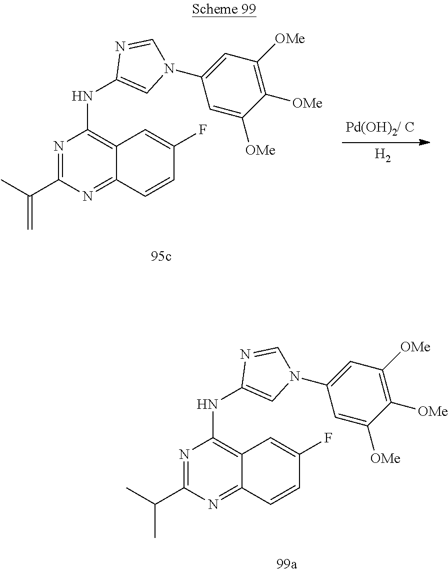 [SMILES: C=C(C)c1nc(Nc2cn(-c3cc(OC)c(OC)c(OC)c3)cn2)c2cc(F)ccc2n1 ; "H2" ; COc1cc(-n2cnc(Nc3nc(C(C)C)nc4ccc(F)cc34)c2)cc(OC)c1OC]

### Preparation of 6-fluoro-2-isopropyl-N-(1-(3,4,5-trimethoxyphenyl)-1H-imidazol-4-yl)quinazolin-4-amine (99a)

Compound 99a was prepared according to the procedure reported in step-3 of scheme 1, from 6-fluoro-2-(prop-1-en-2-yl)-N-(1-(3,4,5-trimethoxyphenyl)-1H-imidazol-4-yl)quinazolin-4-amine (95c) (0.25 g, 0.574 mmol) in MeOH:DCM (20 mL, Ratio: 10:2) using 50% wet, 20% Pd(OH)₂ on carbon (0.0161 mg, 0.0113 mmol) and stirring for 12 h at RT under a H₂ atmosphere. This gave after work up and recrystallization using MeOH (10 mL) 6-fluoro-2-isopropyl-N-(1-(3,4,5-trimethoxyphenyl)-1H-imidazol-4-yl)quinazolin-4-amine (99a) (100 mg, 40% yield) as an yellow solid; ¹H NMR (300 MHz, DMSO-d₆+D₂O) δ 8.69 (d, J=9.9 Hz, 1H), 8.35 (s, 1H), 8.27 (s, 1H), 7.93-7.84 (m, 2H), 6.96 (s, 2H), 3.88 (s, 6H), 3.71 (s, 3H), 3.24 (p, J=6.9 Hz, 1H), 1.43 (d, J=6.8 Hz, 6H); MS (ES+): 438.3 (M+1), (ES−): 436.3 (M−1).

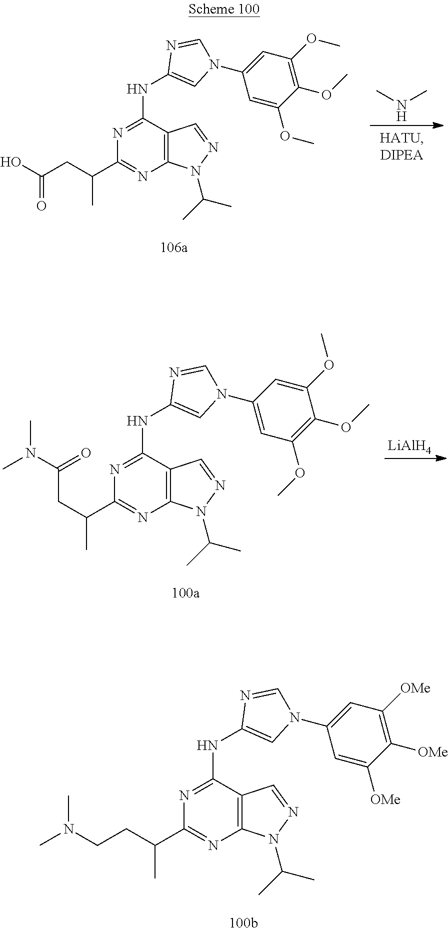

### Preparation of 6-(4-(dimethylamino)butan-2-yl)-1-isopropyl-N-(1-(3,4,5-trimethoxyphenyl)-1H-imidazol-4-yl)-1H-pyrazolo[3,4-d]pyrimidin-4-amine (100b)

### Step-1: Preparation of 3-(1-isopropyl-4-((1-(3,4,5-trimethoxyphenyl)-1H-imidazol-4-yl)amino)-1H-pyrazolo[3,4-d]pyrimidin-6-yl)-N,N-dimethylbutanamide (100a)

Compound 100a was prepared according to the procedure reported in scheme 107, from 3-(1-isopropyl-4-((1-(3,4,5-trimethoxyphenyl)-1H-imidazol-4-yl)amino)-1H-pyrazolo[3,4-d]pyrimidin-6-yl)butanoic acid (106a) (0.2 g, 0.40 mmol) in DMF (4.0 mL) using HATU (0.23 g, 0.61 mmol), DIPEA (0.21 mL, 1.21 mmol), dimethylamine in THF (0.043 g, 0.61 mmol, 2 M) and stirring at RT for 15 h. This gave after work up and purification using column chromatography [silica gel, eluting with MeOH in DCM from 0-5%] to afford 3-(1-isopropyl-4-((1-(3,4,5-trimethoxyphenyl)-1H-imidazol-4-yl)amino)-1H-pyrazolo[3,4-d]pyrimidin-6-yl)-N,N-dimethylbutanamide (100a) (160 mg, 77% yield) as a light brown solid; ¹H NMR (300 MHz, DMSO-d₆) δ 9.99 (s, 1H), 7.50 (s, 1H), 7.35 (s, 1H), 7.20 (s, 1H), 6.10 (s, 2H), 4.20-4.05 (m, 1H), 3.00 (s, 6H), 2.81 (s, 3H), 2.10 (dd, J=15.3, 7.2 Hz, 1H), 1.99 (s, 3H), 1.85 (s, 3H), 1.72-1.64 (m, 2H), 0.57 (t, J=5.9 Hz, 6H), 0.49 (d, J=6.9 Hz, 3H).

### Step-2: Preparation of 6-(4-(dimethylamino)butan-2-yl)-1-isopropyl-N-(1-(3,4,5-trimethoxyphenyl)-1H-imidazol-4-yl)-1H-pyrazolo[3,4-d]pyrimidin-4-amine (100b)

To a stirred solution of 3-(1-isopropyl-4-((1-(3,4,5-trimethoxyphenyl)-1H-imidazol-4-yl)amino)-1H-pyrazolo[3,4-d]pyrimidin-6-yl)-N,N-dimethylbutanamide (100a) (0.2 g, 0.38 mmol) in THF (10 mL) at 0° C. was added LiAlH₄ (2.5 M in THF, 0.3 mL, 0.77 mmol) and heated at reflux for 16 h. The reaction mixture was cooled to 0° C. additional LiAlH₄ (2.5 M in THF, 0.15 mL, 0.38 mmol) was added and heated at reflux for 9.0 h. The reaction mixture was cooled to room temperature, diluted with ethyl acetate (50 mL) added Na₂SO₄ (2.0 g) and stirred for 30 minutes. The slurry was filtered through a pad of Celite and washed with ethyl acetate (20 mL). Filtrate was concentrated and purified using reverse phase column chromatography [eluting with ACN in water (containing 0.1% HCl)] to afford 6-(4-(dimethylamino)butan-2-yl)-1-isopropyl-N-(1-(3,4,5-trimethoxyphenyl)-1H-imidazol-4-yl)-1H-pyrazolo[3,4-d]pyrimidin-4-amine (100b) (66 mg, 32%) HCl salt as a light yellow solid; ¹H NMR (300 MHz, DMSO-d₆) δ 11.16 (s, 1H), 10.17 (s, 1H), 8.42 (s, 2H), 8.09 (d, J=1.6 Hz, 1H), 6.99 (s, 2H), 5.15-4.94 (m, 1H), 3.89 (s, 6H), 3.70 (s, 3H), 3.21-2.86 (m, 3H), 2.79-2.63 (m, 6H), 2.33-2.13 (m, 1H), 2.09-1.86 (m, 1H), 1.47 (dd, J=6.7, 2.3 Hz, 6H), 1.38 (d, J=6.8 Hz, 3H); MS (ES+): 509.3 (M+1); (ES−): 507.2 (M−1); Analysis calculated for C₂₆H₃₆N₈O₃·2.45HCl·3.5H₂O: C, 47.24; H, 6.93; Cl, 13.14; N, 16.95; Found: C, 47.30; H, 6.67; Cl, 12.92; N, 16.63.

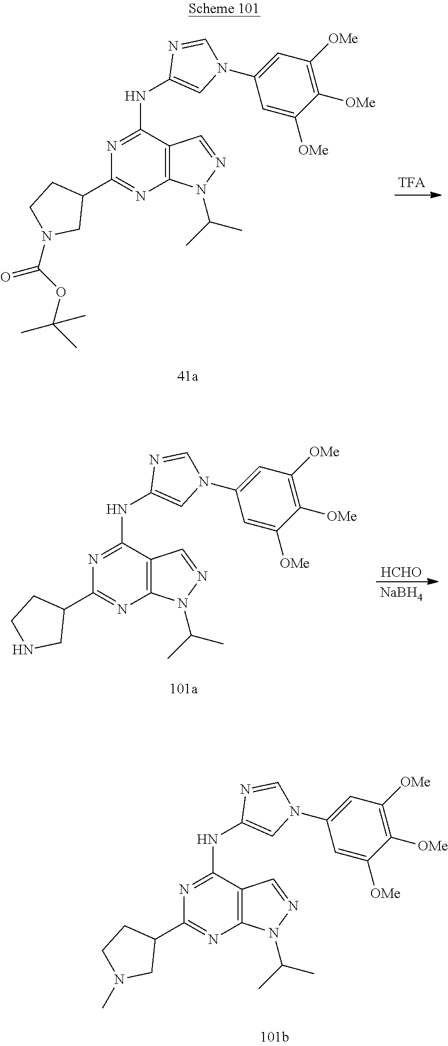 [SMILES: COc1cc(-n2cnc(Nc3nc(C4CCN(C(=O)OC(C)(C)C)C4)nc4c3cnn4C(C)C)c2)cc(OC)c1OC ; COc1cc(-n2cnc(Nc3nc(C4CCNC4)nc4c3cnn4C(C)C)c2)cc(OC)c1OC ; "HCHO" ; "NaBH4" ; COc1cc(-n2cnc(Nc3nc(C4CCN(C)C4)nc4c3cnn4C(C)C)c2)cc(OC)c1OC]

### Preparation of 1-isopropyl-6-(1-methylpyrrolidin-3-yl)-N-(1-(3,4,5-trimethoxyphenyl)-1H-imidazol-4-yl)-1H-pyrazolo[3,44d]pyrimidin-4-amine (101b)

### Step-1: Preparation of 1-isopropyl-6-(pyrrolidin-3-yl)-N-(1-(3,4,5-trimethoxyphenyl)-1H-imidazol-4-yl)-1H-pyrazolo[3,4-d]pyrimidin-4-amine (11a)

To a solution of tert-butyl 3-(1-isopropyl-4-((1-(3,4,5-trimethoxyphenyl)-1H-imidazol-4-yl)amino)-1H-pyrazolo[3,4d]pyrimidin-6-yl)pyrrolidine-1-carboxylate (41a) (0.1 g, 0.173 mmol) in DCM (5 mL) was added TFA (466 μL) and the reaction mixture was stirred at RT for 14 h. The residue obtained was purified using flash column chromatography [silica gel (12 g), eluting with CMA-80 in DCM from 0-100%] to furnish 1-isopropyl-6-(pyrrolidin-3-yl)-N-(1-(3,4,5-trimethoxyphenyl)-1H-imidazol-4-yl)-1H-pyrazolo[3,4-d]pyrimidin-4-amine (11a) (82.7 mg, 100% yield) as a yellow wax; MS (ES+): 479.5 (M+1).

### Step-2: Preparation of 1-isopropyl-6-(1-methylpyrrolidin-3-yl)-N-(1-(3,4,5-trimethoxyphenyl)-1H-imidazol-4-yl)-1H-pyrazolo[3,44]pyrimidin-4-amine (101b)

To a stirred solution of 1-isopropyl-6-(pyrrolidin-3-yl)-N-(1-(3,4,5-trimethoxyphenyl)-1H-imidazol-4-yl)-1H-pyrazolo[3,4-d]pyrimidin-4-amine (101a) (82.0 mg, 0.171 mmol) in MeOH (3 mL) at 0° C. was added formaldehyde (5.66 mg, 0.188 mmol) and stirred for 30 minutes. To this mixture was added sodium borohydride (13.0 mg, 0.343 mmol) and stirred for 1 h at 0-5° C. Due to incomplete conversion, additional amount of formaldehyde (5.66 mg, 0.188 mmol) and sodium borohydride (13.0 mg, 0.343 mmol) were added and the reaction mixture was slowly warmed to RT overnight. Excess solvent was evaporated and the residue obtained was purified using reverse-phase column chromatography [C-18 column (35 g), eluting with 0.1% aqueous HCl in water and acetonitrile from 0-100%] to afford 1-isopropyl-6-(1-methylpyrrolidin-3-yl)-N-(1-(3,4,5-trimethoxyphenyl)-1H-imidazol-4-yl)-1H-pyrazolo[3,4-d]pyrimidin-4-amine (101b) (0.021 g, 25%) HCl salt as an off-white solid; ¹H NMR (300 MHz, DMSO-d₆) δ 11.05 (s, 1H), 10.91-10.34 (m, 1H), 8.54-8.25 (m, 2H), 8.06-7.89 (m, 1H), 7.00 (s, 2H), 5.15-4.98 (m, 1H), 4.16-3.93 (m, 1H), 3.92-3.87 (m, 6H), 3.70 (s, 4H), 3.58-3.40 (m, 2H), 3.27-3.10 (m, 1H), 2.95-2.82 (m, 3H), 2.70-2.52 (m, 2H), 1.47 (d, J=6.7 Hz, 6H); MS (ES+): 493.4 (M+1).

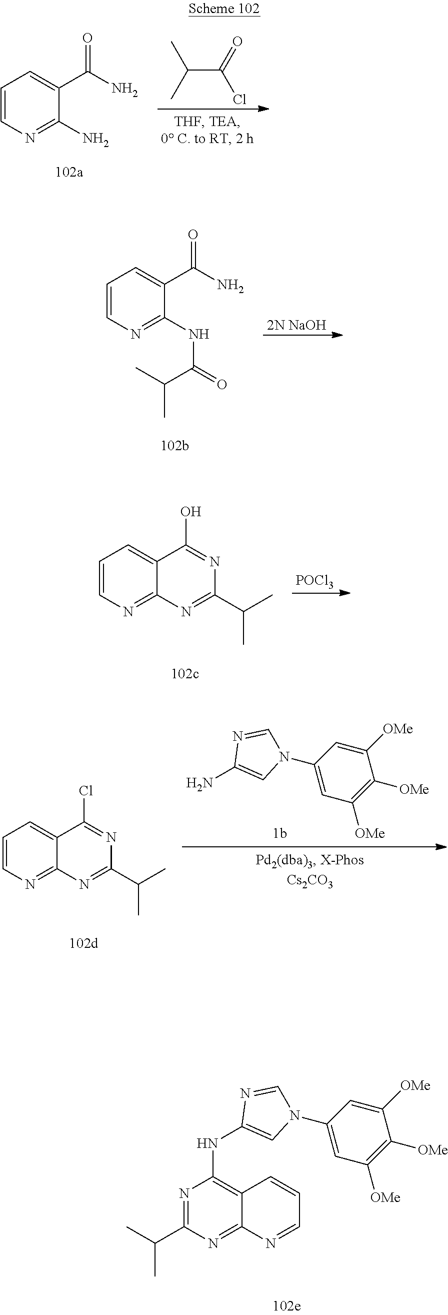

### Preparation of 2-isopropyl-N-(1-(3,4,5-trimethoxyphenyl)-1H-imidazol-4-yl)pyrido[2,3-d]pyrimidin-4-amine (102e)

### Step-1: Preparation of 2-isobutyramidonicotinamide (102b)

To a stirred solution of 2-aminonicotinamide (102a) (1.3 g, 9.48 mmol, CAS #13438-65-8) in THF (41.6 mL) was added triethylamine (1.438 g, 14.22 mmol), cooled to 0° C., added isobutyryl chloride (1.12 g, 10.52 mmol) and stirred at 0° C. for 2 h. The reaction mixture was quenched with water (100 mL) and extracted with DCM (2×100 mL). The combined organic layers were washed with brine (100 mL), dried, filtered and concentrated to give 2-isobutyramidonicotinamide (102b) (0.7 g, 27% yield) as a white solid. ¹H NMR (300 MHz, DMSO-d₆) δ 10.73 (s, 1H), 8.68 (dd, J=4.9, 1.9 Hz, 1H), 8.31 (dd, J=7.8, 1.9 Hz, 1H), 7.42 (dd, J=7.8, 4.9 Hz, 1H), 2.71 (p, J=6.9 Hz, 1H), 1.13 (d, J=6.8 Hz, 6H).

### Step-2: Preparation of 2-isopropylpyrido[2,3-d]pyrimidin-4-ol (102c)

Compound 102c was prepared according to the procedure reported in step-2 of scheme 27, from 2-isobutyramidonicotinamide (102b) (0.6 g, 2.16 mmol) using a solution of NaOH (2N, 24 mL, 12 mmol) and heating at 80° C. for 1 h. This gave after work up 2-isopropylpyrido[2,3-d]pyrimidin-4-ol (102c) (0.40 g, 98% yield) as white solid. ¹H NMR (300 MHz, DMSO-d₆) δ 12.43 (s, 1H), 8.91 (dd, J=4.6, 2.1 Hz, 1H), 8.46 (dd, J=7.8, 2.1 Hz, 1H), 7.49 (ddd, J=6.6, 4.6, 1.8 Hz, 1H), 3.03-2.84 (m, 1H), 1.27 (dd, J=6.8, 2.0 Hz, 6H).

### Step-3: Preparation of 4-chloro-2-isopropylpyrido[2,3-d]pyrimidine (102d)

Compound 102d was prepared according to the procedure reported in step-3 of scheme 27, from 2-isopropylpyrido[2,3-d]pyrimidin-4-ol (102c) (0.35 g, 1.85 mmol) using POCl₃ (8.51 g, 55.49 mmol) and heating at 110° C. for 1 h. This gave after work up 4-chloro-2-isopropylpyrido[2,3-d]pyrimidine (102d) (0.4 g, 100% yield) as a reddish liquid. ¹H NMR (300 MHz, DMSO-d₆) δ 9.11-8.92 (m, 1H), 8.77-8.64 (m, 1H), 7.69 (dd, J=7.8, 4.8 Hz, 1H), 3.05 (p, J=6.4 Hz, 1H), 1.49-1.22 (m, 6H).

### Step-4: Preparation of 2-isopropyl-N-(1-(3,4,5-trimethoxyphenyl)-1H-imidazol-4-yl)pyrido[2,3-d]pyrimidin-4-amine (102e)

Compound 102e was prepared according to the procedure reported in step-4 of scheme 27, from 4-chloro-2-isopropylpyrido[2,3-d]pyrimidine (102d) (0.24 g, 1.16 mmol) in 1,4-dioxane (7.2 mL) using 1-(3,4,5-trimethoxyphenyl)-1H-imidazol-4-amine (1b) (0.576 g, 2.31 mmol), Pd₂(dba)₃ (0.158 g, 0.17 mmol), X-phos (0.22 g, 0.46 mmol), Cs₂CO₃ (1.129 g, 3.47 mmol) and heating at 110° C. for 16 h. This gave after work up and purification using column chromatography [silica gel, eluting with MeOH in DCM from 0-2%] 2-isopropyl-N-(1-(3,4,5-trimethoxyphenyl)-1H-imidazol-4-yl)pyrido[2,3-d]pyrimidin-4-amine (102e) (0.140 g, 29% yield) as an off white solid; ¹H NMR (300 MHz, DMSO-d₆) δ 10.94 (s, 1H), 9.14 (d, J=8.2 Hz, 1H), 8.99 (dd. J=4.4, 1.7 Hz, 1H), 8.27 (s, 2H), 7.53 (dd, J=8.2, 4.4 Hz, 1H), 6.95 (s, 2H), 3.89 (s, 6H), 3.71 (s, 3H), 3.17 (p, J=6.9 Hz, 1H), 1.41 (d, J=6.8 Hz, 6H), MS (ES+): 421.30 (M+1); (ES−): 419.10 (M−1).

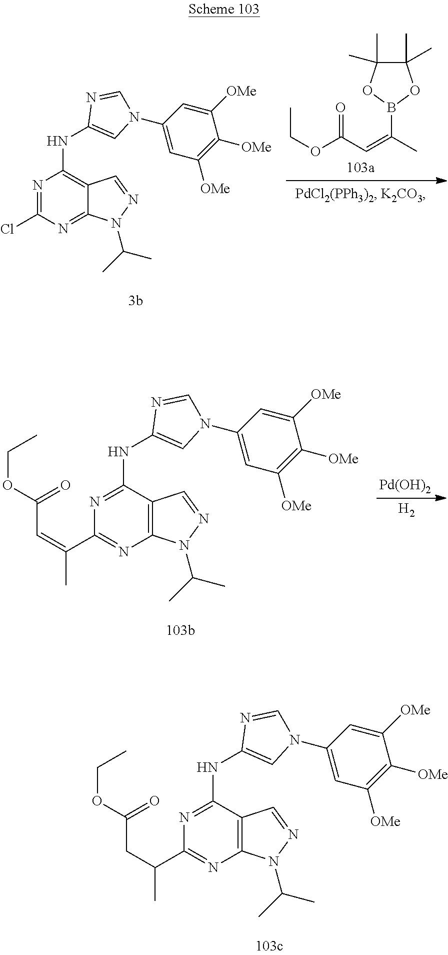

### Preparation of ethyl 3-(1-isopropyl-4-((1-(3,4,5-trimethoxyphenyl)-1H-imidazol-4-yl)amino)-1H-pyrazolo[3,4-d]pyrimidin-6-yl)butanoate (103c)

### Step-1: Preparation of (Z)-ethyl 3-(1-isopropyl-4-((1-(3,4,5-trimethoxyphenyl)-1H-imidazol-4-yl)amino)-1H-pyrazolo[3,4-d]pyrimidin-6-yl)but-2-enoate (103b)

Compound 103b was prepared according to the procedure reported in step-2 of scheme 3, from 6-chloro-1-isopropyl-N-(1-(1-(3,4,5-trimethoxphenyl)-H-imidazol-4-yl)-1H-pyrazolo[3,4-d]pyrimidin-4-amine (3b) (4.0 g, 9.01 mmol) in 1,4-dioxane (80 mL) using (E)-ethyl 3-(4,4,5,5-tetramethyl-1,3,2-dioxaborolan-2-yl)but-2-enoate (103a) (3.89 g, 16.2 mmol), a solution of potassium carbonate (3.73 g, 27.03 mmol) in water (8.0 mL), bis(triphenylphosphine)palladium(II) chloride (1.26 g, 1.80 mmol) and stirring at 100° C. for 12 h under argon. This gave after work up and purification using flash column chromatography [silica gel, eluting with methanol in DCM from 0 to 7%] (Z)-ethyl 3-(1-isopropyl-4-((1-(3,4,5-trimethoxyphenyl)-1H-imidazol-4-yl)amino)-1H-pyrazolo[3,4-d]pyrimidin-6-yl)but-2-enoate (103b) (2.9 g, 62% yield) as an off white solid; ¹H NMR (300 MHz, DMSO-d₆) δ 11.03 (s, 1H), 8.48 (s, 1H), 8.24 (d, J=1.5 Hz, 1H), 8.05 (s, 1H), 7.27 (q, J=1.3 Hz, 1H), 6.95 (s, 2H), 5.26-4.98 (m, 1H), 4.17 (q, J=7.1 Hz, 2H), 3.88 (s, 6H), 3.70 (s, 3H), 2.68 (d, J=1.4 Hz, 3H), 1.50 (d, J=6.6 Hz, 6H), 1.22 (t, J=7.1 Hz, 3H).

### Step-2: Preparation of ethyl 3-(1-isopropyl-4-((1-(3,4,5-trimethoxyphenyl)-1H-imidazol-4-yl)amino)-1H-pyrazolo[3,4-d]pyrimidin-6-yl)butanoate (103c)

Compound 103c was prepared according to the procedure reported in step-3 of scheme 1, from (Z)-ethyl 3-(1-isopropyl-4-((1-(3,4,5-trimethoxyphenyl)-1H-imidazol-4-yl)amino)-1H-pyrazolo[3,4-d]pyrimidin-6-yl)but-2-enoate (103b) (2.6 g, 4.98 mmol) in MeOH (260 mL), DCM (26 mL) and acetic acid (2 mL) using 50% wet, 20% Pd(OH)₂ on carbon (2.8 g, 1.99 mmol) and stirring at RT for 96 h under a H₂ atmosphere. This gave after work up and purification using column chromatography [silica gel, eluting with MeOH in DCM (from 0-4^{%})] ethyl 3-(1-isopropyl-4-((1-(3,4,5-trimethoxyphenyl)-1H-imidazol-4-yl)amino)-1H-pyrazolo[3,4-d]pyrimidin-6-yl)butanoate (103c) (2.4 g, 92% yield) as an off white solid; 1H NMR (300 MHz, DMSO-d₆) δ 10.84 (s, 1H, D₂O exchangeable), 8.39 (s, 1H), 8.23 (s, 1H), 8.07 (s, 1H), 6.99 (s, 2H), 5.11-4.89 (m, 1H), 4.00-3.91 (m, 2H), 3.89 (s, 6H), 3.69 (s, 3H), 3.47-3.36 (m, 1H), 2.99 (dd, J=15.7, 8.8 Hz, 1H), 2.69 (dd, J=15.8, 6.1 Hz, 1H), 1.45 (dd, J=6.7, 2.0 Hz, 6H), 1.36 (d, J=7.1 Hz, 3H), 0.95 (t, J=7.1 Hz, 3H); MS (ES+): 523.80 (M+1); (ES−): 521.80 (M−1).

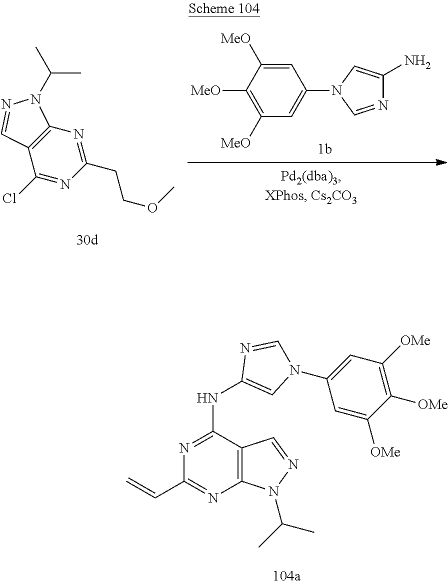 [SMILES: COCCc1nc(Cl)c2cnn(C(C)C)c2n1 ; COc1cc(-n2cnc(N)c2)cc(OC)c1OC ; C=Cc1nc(Nc2cn(-c3cc(OC)c(OC)c(OC)c3)cn2)c2cnn(C(C)C)c2n1]

### Preparation of 1-isopropyl-N-(1-(3,4,5-trimethoxyphenyl)-1H-imidazol-4-yl)-6-vinyl-1H-pyrazolo[3,4-d]pyrimidin-4-amine (104a)

Compound 104a was prepared according to the procedure reported in step-4 of scheme 27, from 4-chloro-1-isopropyl-6-(2-methoxyethyl)-1H-pyrazolo[3,4-d]pyrimidine (30d) (0.35 g, 1.37 mmol) in 1,4-dioxane (10.5 mL) using 1-(3,4,5-trimethoxyphenyl)-1H-imidazol-4-amine (1b) (0.45 g, 1.81 mmol), Pd₂(dba)₃ (0.25 g, 0.27 mmol), X-phos (0.26 g, 0.54 mmol), Cs₂CO₃ (1.34 g, g, 4.12 mmol) and heating at 110° C. for 4 h. This gave after work up and purification using column chromatography [silica gel, eluting with MeOH in DCM from 0-10%] 1-isopropyl-N-(1-(3,4,5-trimethoxyphenyl)-1H-imidazol-4-yl)-6-vinyl-1H-pyrazolo[3,4-d]pyrimidin-4-amine (104a) (0.3 g, 50% yield) as an off-white solid; ¹H NMR (300 MHz, DMSO-d₆) δ 10.89 (s, 1H, D₂O exchangeable), 8.44 (s, 1H), 8.21 (s, 1H), 8.09 (s, 1H), 6.96 (s, 2H), 6.82 (dd, J=17.2, 10.3 Hz, 1H), 6.67-6.51 (m, 1H), 5.84-5.68 (m, 1H), 5.13-4.96 (m, 1H), 3.88 (s, 6H), 3.69 (s, 3H), 1.46 (d, J=6.6 Hz, 6H).

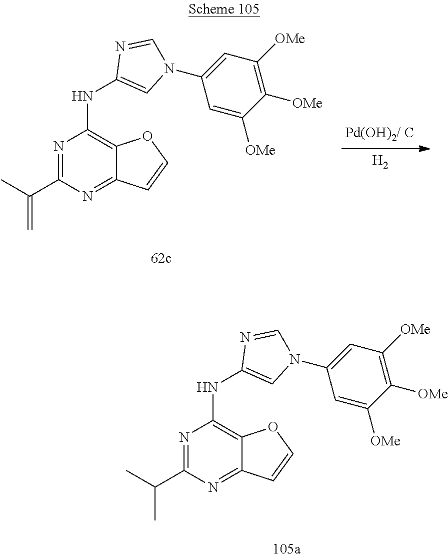 [SMILES: C=C(C)c1nc(Nc2cn(-c3cc(OC)c(OC)c(OC)c3)cn2)c2occc2n1 ; "H2" ; COc1cc(-n2cnc(Nc3nc(C(C)C)nc4ccoc34)c2)cc(OC)c1OC]

### Preparation of 2-isopropyl-N-(1-(3,4,5-trimethoxyphenyl)-1H-imidazol-4-yl)-4a,7a-dihydrofuro[3,2-d]pyrimidin-4-amine (105a)

Compound 105a was prepared according to the procedure reported in step-3 of scheme 1, from 2-(prop-1-en-2-yl)-N-(1-(3,4,5-trimethoxyphenyl)-1H-imidazol-4-yl)furo[3,2-d]pyrimidin-4-amine (62c) (0.2 g, 0.49 mmol) in MeOH and DCM (20 mL, ratio 10:2) using 50% wet, 20% Pd(OH)₂ on carbon (10 mg, 0.0071 mmol) and stirring for 12 h at RT under a H₂ atmosphere. The reaction mixture was filtered through a pad of Celite and the filtrate was concentrated in vacuum. The obtained residue was crystallized using MeOH (10 mL) to give 2-isopropyl-N-(1-(3,4,5-trimethoxyphenyl)-1H-imidazol-4-yl)-4a,7a-dihydrofuro[3,2-d]pyrimidin-4-amine (105a) (0.08 g, 40% yield) as an white solid; ¹H NMR (300 MHz, DMSO-d₆) δ 10.56 (s, 1H, D₂O exchangeable), 8.28 (d, J=2.2 Hz, 1H), 8.20 (d, J=1.6 Hz, 1H) 8.11 (d, J=1.6 Hz, 1H), 7.00 (d, J=2.2 Hz, 1H), 6.93 (s, 2H), 3.87 (s, 6H), 3.69 (s, 3H), 3.20-3.02 (m, 1H), 1.35 (d, J=6.9 Hz, 6H); MS (ES+): 410.20 (M+1); Analysis calculated for: C₂₁H₂₅N₅O₄: C, 61.30; H, 6.12; N, 17.02. Found: C, 61.32; H, 5.72; N, 17.03.

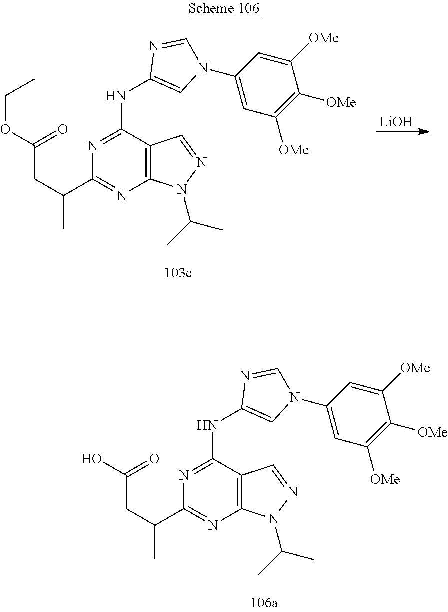

### Preparation of 3-(1-isopropyl-4-((1-(3,4,5-trimethoxyphenyl)-1H-imidazol-4-yl)amino)-1H-pyrazolo[3,4-d]pyrimidin-6-yl)butanoic acid (106a)

To stirred a solution of ethyl 3-(1-isopropyl-4-((1-(3,4,5-trimethoxyphenyl)-1H-imidazol-4-yl)amino)-1H-pyrazolo[3,4-d]pyrimidin-6-yl)butanoate (103c) (0.15 g, 0.286 mmol) in THF (2.25 mL) and MeOH (2.25 mL) was added LiOH·H₂O (0.036 g, 0.85 mmol) in water (0.75 mL) and stirred at RT for 16 h. The reaction mixture was diluted with water (300 mL) and pH was adjusted to 6.0 using 1N HCl and extracted with ethyl acetate (2×100 mL). The combined organics were washed with brine (100 mL) dried, filtered and concentrated to obtain an off-white solid residue (2.3 g). Acetone 10.0 mL was added to the residue obtained and stirred for 30 min at RT and the resulted solid was filtered to give 3-(1-isopropyl-4-((1-(3,4,5-trimethoxyphenyl)-1H-imidazol-4-yl)amino)-1H-pyrazolo[3,4-d]pyrimidin-6-yl)butanoic acid (106a) (75 mg, 53% yield) hydrochloride salt as an tan solid; ¹H NMR (300 MHz, DMSO-d₆) δ 12.08 (s, 1H), 10.83 (s, 1H), 8.39 (s, 1H), 8.23 (s, 1H), 8.10 (s, 1H), 6.99 (s, 2H), 5.10-4.93 (m, 1H), 3.88 (s, 6H), 3.69 (s, 3H), 3.47-3.36 (m, 1H), 2.96 (dd, J=15.9, 8.5 Hz, 1H), 2.61 (dd, J=15.9, 6.3 Hz, 1H), 1.45 (dd, J=6.7, 3.1 Hz, 6H), 1.33 (d, J=7.0 Hz, 3H); MS (ES+): 495.8 (M+1); (ES−): 493.8 (M−1); Analysis calculated for: C₂₄H₂₉N₇O₅.0.5HCl·0.75H₂O: C, 54.67; H, 5.93; N, 18.60; Found: C, 54.86; H, 5.83; N, 18.27.

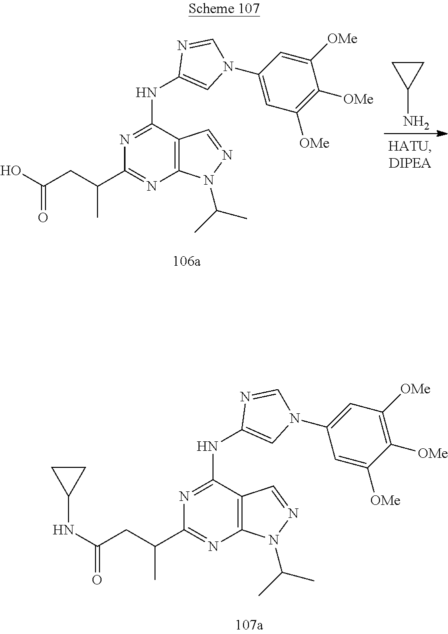

### Preparation of N-cyclopropyl-3-(1-isopropyl-4-((1-(3,4,5-trimethoxyphenyl)-1H-imidazol-4-yl)amino)-1H-pyrazolo[3,4-d]pyrimidin-6-yl)butanamide (107a)

To a stirred solution of 3-(1-isopropyl-4-((1-(3,4,5-trimethoxyphenyl)-1H-imidazol-4-yl)amino)-1H-pyrazolo[3,4-d]pyrimidin-6-yl)butanoic acid (106a) (0.25 g, 0.50 mmol) in DMF (5 mL) at 0° C. was added HATU (0.287 g, 0.756 mmol), DIPEA (0.195 g, 1.512 mmol) and stirred at 0° C. for 30 minutes followed by the addition of cyclopropylamine (0.043 g, 0.756 mmol). The reaction mixture was allowed to warm to room temperature over a 15 h period and diluted with water (18.0 mL). The solid obtained was collected by filtration, dried to afford N-cyclopropyl-3-(1-isopropyl-4-((1-(3,4,5-trimethoxyphenyl)-1H-imidazol-4-yl)amino)-1H-pyrazolo[3,4-d]pyrimidin-6-yl)butanamide (107a) (230 mg, 85% yield) as an off white solid; ¹H NMR (300 MHz, DMSO-d) δ 10.81 (s, 1H, D₂O exchangeable), 8.38 (s, 1H), 8.23 (d, J=1.5 Hz, 1H), 8.16 (s, 1H), 7.90 (d, J=4.1 Hz, 1H), 7.03 (s, 2H), 5.12-4.94 (m, 1H), 3.89 (s, 6H), 3.69 (s, 3H), 3.51-3.37 (m, 1H), 2.65-2.55 (m, 2H), 2.41-2.18 (m, 1H), 1.45 (d, J=6.7 Hz, 6H), 1.33 (d, J=6.9 Hz, 3H), 0.60-0.40 (m, 2H), 0.36-0.15 (m, 2H); MS (ES+): 535.30 (M+1).

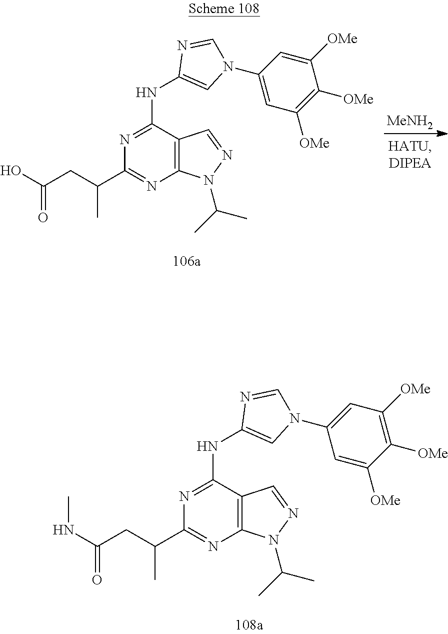 [SMILES: "MeNH2" ; COc1cc(-n2cnc(Nc3nc(C(C)CC(=O)O)nc4c3cnn4C(C)C)c2)cc(OC)c1OC ; CNC(=O)CC(C)c1nc(Nc2cn(-c3cc(OC)c(OC)c(OC)c3)cn2)c2cnn(C(C)C)c2n1]

### Preparation of 3-(1-isopropyl-4-((1-(3,4,5-trimethoxyphenyl)-1H-imidazol-4-yl)amino)-1H-pyrazolo[3,4-d]pyrimidin-6-yl)-N-methylbutanamide (108a)

Compound 108a was prepared according to the procedure reported in scheme 107, from 3-(1-isopropyl-4-((1-(3,4,5-trimethoxyphenyl)-1H-imidazol-4-yl)amino)-1H-pyrazolo[3,4-d]pyrimidin-6-yl)butanoic acid (106a) (0.25 g, 0.50 mmol) in DMF (5.0 mL) using HATU (0.287 g, 0.756 mmol), DIPEA (0.195 g, 1.51 mmol), 7% methylamine in THF (0.2 mL, 0.756 mmol) and stirring at RT for 15 h. This gave after work up and trituration with ether 3-(1-isopropyl-4-((1-(3,4,5-trimethoxyphenyl)-1H-imidazol-4-yl)amino)-1H-pyrazolo[3,4-d]pyrimidin-6-yl)-N-methylbutanamide (108a) (200 mg, 79% yield) as a brown solid; ¹H NMR (300 MHz, DMSO-d₆) δ 10.81 (s, 1H, D₂O exchangeable), 8.38 (s, 1H), 8.24 (d, J=1.5 Hz, 1H), 8.15 (s, 1H), 7.82-7.73 (m, 1H), 7.02 (s, 2H), 5.08-4.96 (m, 1H), 3.89 (s, 6H), 3.69 (s, 3H), 3.46-3.36 (m, 1H), 2.70-2.57 (m, 2H, 1H D₂O exchangeable), 2.51 (s, 3H), 2.43-2.31 (m, 1H), 1.45 (d, J=6.7 Hz, 6H), 1.34 (d, J=6.9 Hz, 3H); MS (ES+): 509.30 (M+1).

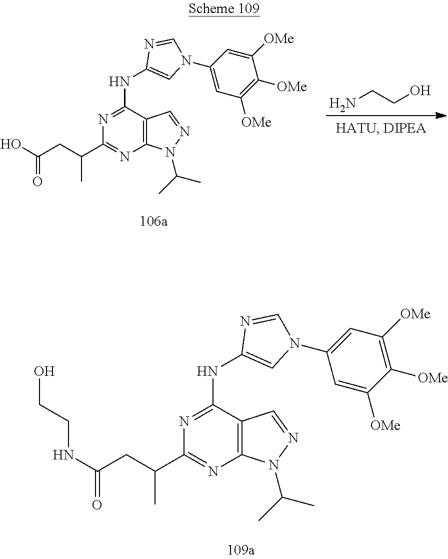

### Preparation of N-(2-hydroxyethyl)-3-(1-isopropyl-4-((1-(3,4,5-trimethoxyphenyl)-1H-imidazol-4-yl)amino)-1H-pyrazolo[3,4-d]pyrimidin-6-yl)butanamide (109a)

Compound 109a was prepared according to the procedure reported in scheme 107, from 3-(1-isopropyl-4-((1-(3,4,5-trimethoxyphenyl)-1H-imidazol-4-yl)amino)-1H-pyrazolo[3,4-d]pyrimidin-6-yl)butanoic acid (106a) (0.25 g, 0.5 mmol) in DMF (5.0 mL) using HATU (0.287 g, 0.756 mmol), DIPEA (0.195 g, 1.51 mmol), ethanolamine (0.046 g, 0.756 mmol) and stirring at RT for 15 h. This gave after work up and purification by column chromatography [silica gel, eluting with MeOH in DCM from 0-5%] to afford N-(2-hydroxyethyl)-3-(1-isopropyl-4-((1-(3,4,5-trimethoxyphenyl)-1H-imidazol-4-yl)amino)-1H-pyrazolo[3,4-d]pyrimidin-6-yl)butanamide (109a) (180 mg, 67% yield) as an off white solid; ¹H NMR (300 MHz, DMSO-d₆) δ 10.82 (s, 1H), 8.39 (s, 1H), 8.24 (s, 1H), 8.16 (s, 1H), 7.87 (t, J=5.6 Hz, 1H), 7.00 (s, 2H), 5.14-4.97 (m, 1H), 4.60 (t, J=5.4 Hz, 1H), 3.89 (s, 6H), 3.70 (s, 3H), 3.50-3.37 (m, 1H), 3.34-3.24 (m, 2H), 3.16-2.97 (m, 2H), 2.67 (dd, J=13.9, 6.2 Hz, 1H), 2.44 (dd, 1H), 1.46 (d, J=6.7 Hz, 6H), 1.35 (d, J=6.9 Hz, 3H); MS (ES+): 539.30 (M+1); (ES−): 537.30 (M−1).

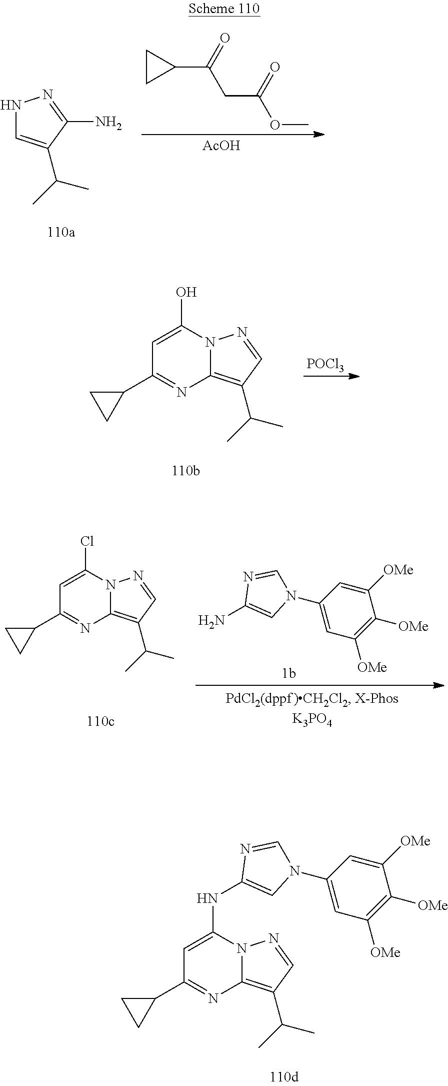

### Preparation of 5-cyclopropyl-3-isopropyl-N-(1-(3,4,5-trimethoxyphenyl)-1H-imidazol-4-yl)pyrazolo[1,5-a]pyrimidin-7-amine (110d)

### Step-1: Preparation of 5-cyclopropyl-3-isopropylpyrazolo[1,5-a]pyrimidin-7-ol (110b)

To a stirred solution of 4-isopropyl-1H-pyrazol-3-amine (110a) (1.5 g, 11.98 mmol, CAS #15152149-2) in acetic acid (7.5 mL) was added methyl 3-cyclopropyl-3-oxopropanoate (1.70 g, 11.96 mmol, CAS #32249-35-7) and heated at 120° C. for 16 h. The reaction was concentrated and azeotroped with toluene to get 5-cyclopropyl-3-isopropylpyrazolo[1,5-a]pyrimidin-7-ol (110b) (2 g, 77%) as a yellow solid; ¹H NMR (300 MHz, DMSO-d₆) δ 11.93 (s, 1H), 7.77 (s, 1H), 5.21 (s, 1H), 3.14 (h, J=6.8 Hz, 1H), 2.03-1.90 (m, 1H), 1.23 (d, J=6.7 Hz, 6H), 1.13-1.04 (m, 2H), 0.99-0.85 (m, 2H).

### Step-2: Preparation of 7-chloro-5-cyclopropyl-3-isopropylpyrazolo[1,5-a]pyrimidine (110c)

Compound 110c was prepared according to the procedure reported in step-3 of scheme 27, from 5-cyclopropyl-3-isopropylpyrazolo[1,5-a]pyrimidin-7-ol (110b) (2 g, 9.21 mmol) using POCl₃ (42.34 g, 276.15 mmol), N,N-dimethylaniline (3.35 g, 27.62 mmol) and heating at 110° C. for 1 h. This gave after work up and purification by column chromatography [silica gel (mesh size: 320-400) eluting with EtOAc in n-heptane (0% to 5%] 7-chloro-5-cyclopropyl-3-isopropylpyrazolo[1,5-a]pyrimidine (110c) (0.7 g, 32% yield) as a yellow solid; ¹H NMR (300 MHz, DMSO-d₆) δ 8.11 (s, 1H), 7.29 (s, 1H), 3.24-3.07 (m, 1H), 2.20 (m, 1H), 1.31 (d, J=6.7 Hz, 6H), 1.15-1.05 (m, 4H).

### Step-3: Preparation of 5-cyclopropyl-3-isopropyl-N-(1-(3,4,5-trimethoxyphenyl)-1H-imidazol-4-yl)pyrazolo[1,5-a]pyrimidin-7-amine (110d)

Compound 110d was prepared according to the procedure reported in step-4 of scheme 7, from 7-chloro-5-cyclopropyl-3-isopropylpyrazolo[1,5-a]pyrimidine (110c) (0.4 g, 1.7 mmol) in 1,4-dioxane (12 mL) using 1-(3,4,5-trimethoxyphenyl)-1H-imidazol-4-amine (1b) (0.47 g, 1.89 mmol), XPhos (0.323 g, 0.68 mmol), K₃PO₄ (0.539 g, 2.54 mmol), PdCl₂(dppf)-CH₂Cl₂ adduct (0.21 g, 0.25 mmol) and heating at 110° C. for 16 h. This gave after workup and purification using flash column chromatography [silica gel, eluting with EtOAc in n-heptane from 0-70%] 5-cyclopropyl-3-isopropyl-N-(1-(3,4,5-trimethoxyphenyl)-1H-imidazol-4-yl)pyrazolo[1,5-a]pyrimidin-7-amine (110d) (60 mg, 8% yield) as a brown solid; ¹H NMR (300 MHz, DMSO-d₆) δ 9.63 (s, 1H, D₂O exchangeable), 8.25 (d, J=1.6 Hz, 1H), 7.94 (s, 1H), 7.69 (d, J=1.6 Hz, 1H), 6.97 (s, 2H), 6.76 (s, 1H), 3.88 (s, 6H), 3.69 (s, 3H), 3.18-3.03 (m, 1H), 2.11-1.94 (m, 1H), 1.30 (d, J=6.9 Hz, 6H), 1.02-0.88 (m, 4H); MS (ES+): 449.20 (M+1); Analysis calculated for C₂₄H₂₈N₆O₃: C, 64.63; H, 6.94; N, 18.09; Found: C, 64.62; H, 6.58; N, 17.73.

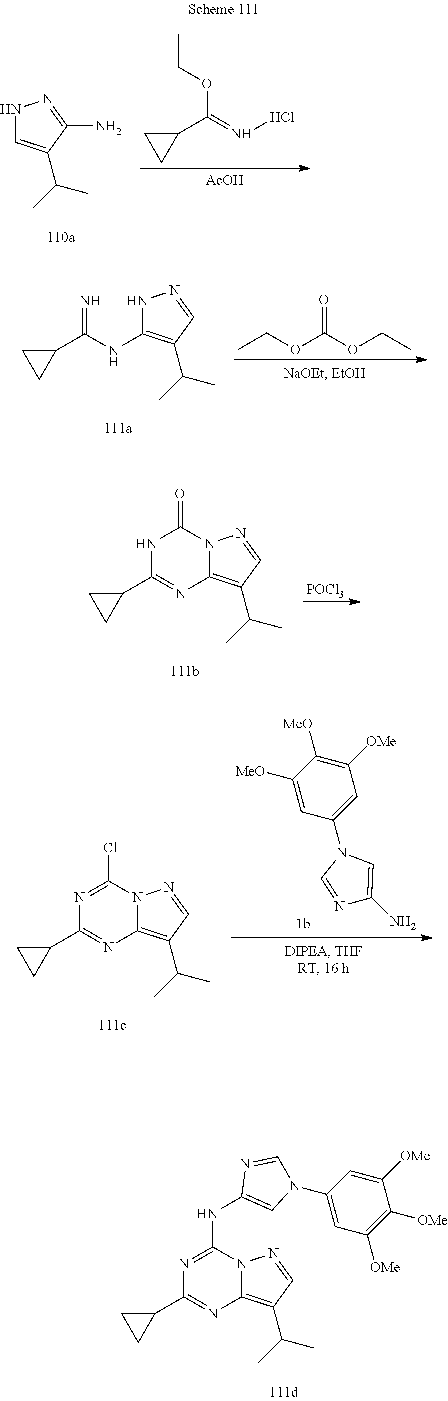

### Preparation of 2-cyclopropyl-8-isopropyl-N-(1-(3,4,5-trimethoxyphenyl)-1H-imidazol-4-yl)pyrazolo[1,5-a][1,3,5]triazin-4-amine (111d)

### Step-1: Preparation of N-(4-isopropyl-1H-pyrazol-5-yl)cyclopropanecarboximidamide (111a)

To a stirred solution of 4-isopropyl-1H-pyrazol-3-amine (110a) (0.5 g, 3.99 mmol) in DCM (15 mL) was added ethylcyclopropanecarbaimidate hydrochloride (1.35 g, 9.02 mmol, CAS #63190-44-3), acetic acid (0.24 g, 3.99 mmol) and stirred at RT for 16 h. The reaction mixture was concentrated in vacuum to N-(4-isopropyl-1H-pyrazol-5-yl)cyclopropanecarboximidamide (111a) (0.76 g, 99% yield) as a sticky liquid, which was used in next step without purification. ¹H NMR (300 MHz, DMSO-d₆) δ 10.01 (s, 1H), 8.00 (s, 1H), 7.02 (s, 1H), 2.54 (m, 1H), 1.37-1.24 (m, 1H), 0.87 (d, J=6.8 Hz, 6H), 0.70-0.58 (m, 2H), 0.54-0.43 (m, 2H).

### Step-2: Preparation of 2-cyclopropyl-8-isopropylpyrazolo[1,5-a][1,3,5]triazin-4 (3H)-one (111b)

To a stirred solution of N-(4-isopropyl-1H-pyrazol-5-yl)cyclopropanecarboximidamide (111a) (0.7 g, 3.64 mmol) in ethanol (25 mL) was added diethylcarbonate (3.44 g, 29.15 mmol), sodium ethoxide (2.48 g, 36.41 mmol) and heated at 80° C. for 16 h. The reaction mixture was concentrated in vacuum and to the residue was added water, pH was adjusted to 6 using IN HCL. The solid obtained was collected by filtration dried to afford 2-cyclopropyl-8-isopropylpyrazolo[1,5-a][1,3,5]triazin-4 (3H)-one (111b) (0.35 g, 44% yield) as cream colored solid; ¹H NMR (300 MHz, DMSO-d) δ 12.53 (s, 1H), 7.90 (s, 1H), 2.95 (hept, J=6.9 Hz, 1H), 1.92 (p, J=6.5 Hz, 1H), 1.21 (d, J=6.9 Hz, 6H), 1.08-1.06 (m, 2H), 1.06-1.03 (m, 2H).

### Step-3: Preparation of 4-chloro-2-cyclopropyl-8-isopropylpyrazolo[1,5-a][1,3,5]triazine (111c)

Compound 111c was prepared according to the procedure reported in step-3 of scheme 27, from 2-cyclopropyl-8-isopropylpyrazolo[1,5-a][1,3,5]triazin-4 (3H)-one (111b) (0.35 g, 1.60 mmol) in toluene (6.7 mL) using POCl₃ (0.98 g, 6.41 mmol) and heating at 100° C. for 4 h. This gave after work up 4-chloro-2-cyclopropyl-8-isopropylpyrazolo[1,5-a][1,3,5]triazine (111c) (0.38 g) as a reddish liquid, which was used in next step without purification.

### Step-4: Preparation of 2-cyclopropyl-8-isopropyl-N-(1-(3,4,5-trimethoxyphenyl)-1H-imidazol-4-yl)pyrazolo[1,5-a][1,3,5]triazin-4-amine (111d)

Compound 111d was prepared according to the procedure reported in step-1 of scheme 1, from 4-chloro-2-cyclopropyl-8-isopropylpyrazolo[1,5-a][1,3,5]triazine (111c) (0.38 g, 1.61 mmol) in THF (9.5 mL) using 1-(3,4,5-trimethoxyphenyl)-1H-imidazol-4-amine (1b) (0.8 g, 3.21 mmol), DIPEA (0.55 g, 4.29 mmol) and stirring at rt for 16 h. This gave after work up 2-cyclopropyl-8-isopropyl-N-(1-(3,4,5-trimethoxyphenyl)-1H-imidazol-4-yl)pyrazolo[1,5-a][1,3,5]triazin-4-amine (111d) (0.71 g, 98% yield) as an off white solid. ¹H NMR (300 MHz, DMSO-d₆) δ 10.41 (s, 1H), 8.21 (s, 1H), 8.07 (s, 1H), 7.88 (s, 1H), 6.96 (s, 2H), 3.89 (s, 6H), 3.70 (s, 3H), 3.12 (p, J=6.9 Hz, 1H), 2.14-2.01 (m, 1H), 1.31 (d, J=6.9 Hz, 6H), 1.20-1.11 (m, 2H), 1.05-0.96 (m, 2H); MS (ES+): 450.3 (M+1), (ES−): 448.4 (M−1); Analysis calculated for: C₂₁H₂₇N₇O₃: C, 61.46; H, 6.05; N, 21.81; Found: C, 61.33; H, 5.99; N, 21.84.

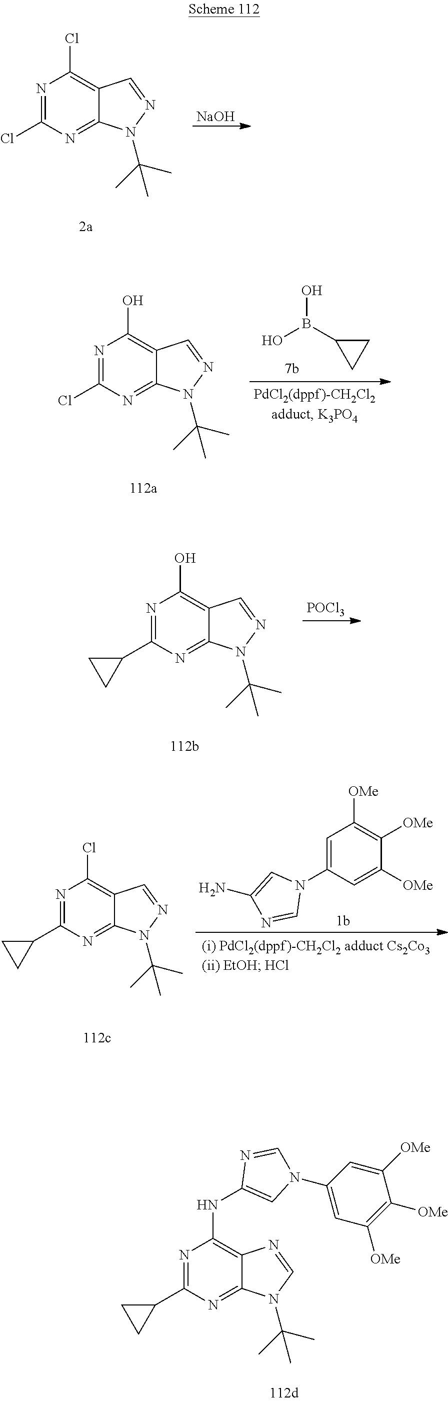

### Preparation of 1-(tert-butyl)-6-cyclopropyl-N-(1-(3,4,5-trimethoxyphenyl)-1H-imidazol-4-yl)-1H-pyrazolo[3,4-d]pyrimidin-4-amine (112d)

### Step-1: Preparation of 1-(tert-butyl)-6-chloro-1H-pyrazolo[3,4-d]pyrimidin-4-ol (112a)

Compound 112a was prepared according to the procedure reported in step-1 of scheme 7, from a solution of 1-tert-butyl)-4,6-dichloro-1H-pyrazolo[3,4-d]pyrimidine (2a) (28.0 g, 114.23 mmol) in NaOH (2N) (22.84 g, 571.0 mmol) and heating at 90° C. for 2 h to afford after work up 1-(tert-butyl)-6-chloro-1H-pyrazolo[3,4-d]pyrimidin-4-ol (112a) (22.0 g, 85% yield) as a white solid, which was used as such for the next step; ¹H NMR (300 MHz, DMSO-d₆) δ 8.02 (d, J=0.7 Hz, 1H), 1.67 (s, 9H).

### Step-2: Preparation of 1-(tert-butyl)-6-cyclopropyl-1H-pyrazolo[3,4-d]pyrimidin-4-ol (112b)

Compound 112b was prepared according to the procedure reported in step-1 of scheme 1, from 1-(tert-butyl)-6-chloro-1H-pyrazolo[3,4-d]pyrimidin-4-ol (112a) (8.0 g, 35.30 mmol) in toluene (160 mL) using cyclopropylboronic acid (7b) (12.12 g, 141.18 mmol), PdCl₂(dppf)-CH₂Cl₂ adduct (5.23 g, 7.05 mmol), a solution of K₃PO₄ (17.15 g, 80.73 mmol) in water (1.28 mL) and heating at 100° C. for 12 h to afford after work up and purification using flash column chromatography [silica gel, eluting with MeOH in DCM from 0-10%] 1-(tert-butyl)-6-cyclopropyl-1H-pyrazolo[3,4-d]pyrimidin-4-ol (112b) (6.0 g, 73% yield) as an off-white solid; ¹H NMR (300 MHz, DMSO-d₆) δ 12.30 (s, 1H), 7.89 (d, J=2.2 Hz, 1H), 2.16-1.91 (m, 1H), 1.64 (s, 9H), 1.20-0.97 (m, 4H).

### Step-3: Preparation of 1-(tert-butyl)-4-chloro-6-cyclopropyl-1H-pyrazolo[3,4-d]pyrimidine (112c)

Compound 112c was prepared according to the procedure reported in step-3 of scheme 7, from 1-(tert-butyl)-6-cyclopropyl-1H-pyrazolo[3,4-d]pyrimidin-4-ol (112b) (5.0 g, 21.52 mmol) using POCl₃ (191.46 g, 1248.43 mmol) and heating at 100° C. for 1 h to afford after work up and purification using flash column chromatography [silica gel, eluting with EtOAc in n-heptane from 0-50%] 1-(tert-butyl)-4-chloro-6-cyclopropyl-1H-pyrazolo[3,4-d]pyrimidine (112c) (4.0 g, 74% yield) as an oily mass; ¹H NMR (300 MHz, DMSO-d₆) δ 8.25 (d, J=1.8 Hz, 1H), 2.35-2.20 (m, 1H), 1.75 (s, 9H), 1.23-1.10 (m, 4H).

### Step-4: Preparation of 1-(tert-butyl)-6-cyclopropyl-N-(1-(3,4,5-trimethoxyphenyl)-1H-imidazol-4-yl)-1H-pyrazolo[3,4-d]pyrimidin-4-amine (112d)

To a stirred a solution of 1-(tert-butyl)-4-chloro-6-cyclopropyl-1H-pyrazolo[3,4-d]pyrimidine (112c) (4.0 g, 15.95 mmol) in 1,4-dioxane (100.0 mL) was added 1-(3,4,5-trimethoxyphenyl)-1H-imidazol-4-amine (1b) (4.37 g, 17.54 mmol), PdCl₂(dppf)-CH₂Cl₂ adduct (0.65 g, 0.79 mmol) and Cs₂CO₃ (15.59 g 47.86 mmol). The reaction mixture was purged with nitrogen gas for 15 min and heated at 100° C. for 12 h. The mixture was cooled to RT, filtered through a pad of Celite and concentrated in vacuo. The residue obtained was purified using flash column chromatography [silica gel, eluting with MeOH in DCM from 0-10%] to give 1-(tert-butyl)-6-cyclopropyl-N-(1-(3,4,5-trimethoxyphenyl)-1H-imidazol-4-yl)-1H-pyrazolo[3,4-d]pyrimidin-4-amine (112d) (2.0 g, 27% yield) free base as a grey solid; ¹H NMR (300 MHz, DMSO-d₆) δ 10.71 (s, 1H), 8.38-8.11 (m, 2H), 7.97 (s, 1H), 6.94 (s, 2H), 3.90 (s, 6H), 3.71 (d, J=1.9 Hz, 3H), 2.16 (s, 1H), 1.72 (s, 9H), 1.24-1.14 (m, 2H), 1.06-0.96 (m, 2H). The free base of compound 112d was converted to its HCl salt by dissolving (1.0 g, 2.16 mmol) in EtOH (4 mL), adding 14% HCl in EtOH (2 mL) and stirring for 1 h at RT. The solid obtained was collected by filtration and dried to afford 1-(tert-butyl)-6-cyclopropyl-N-(1-(3,4,5-trimethoxyphenyl)-1H-imidazol-4-yl)-1H-pyrazolo[3,4-d]pyrimidin-4-amine (112d) (1.05 g, 97% yield) HCl salt as a white solid; ¹H NMR (300 MHz, DMSO-d₆) δ 11.03 (s, 1H, D₂O exchangeable), 8.49 (s, 1H), 8.27 (s, 1H), 8.00 (d, J=1.7 Hz, 1H, D₂O exchangeable), 7.00 (s, 2H), 3.89 (s, 6H), 3.70 (s, 3H), 2.25-2.12 (m, 1H), 1.72 (s, 9H), 1.23-1.12 (m, 2H), 1.09-0.97 (m, 2H); MS (ES+): 464.2 (M+1); (ES−): 462.1 (M−1).

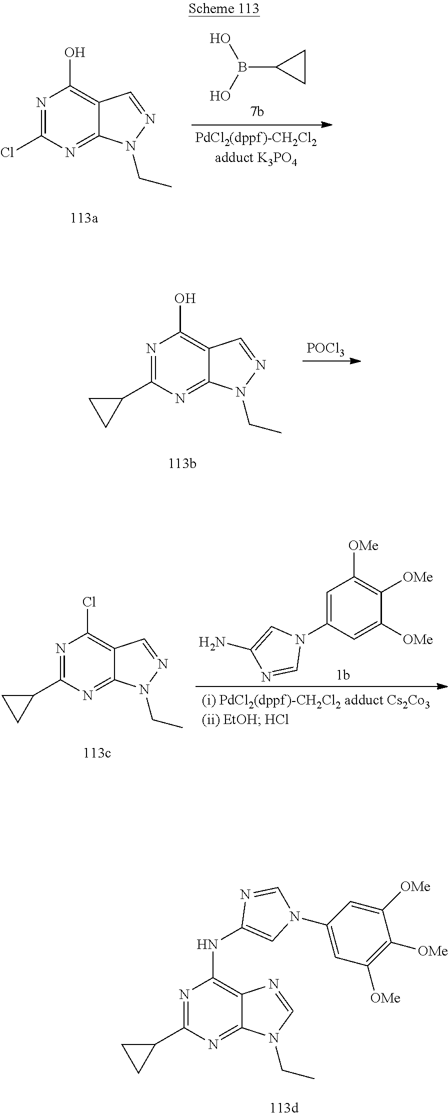

### Preparation of 6-cyclopropyl-1-ethyl-N-(1-(3,4,5-trimethoxyphenyl)-1H-imidazol-4-yl)-1H-pyrazolo[3,4-d]pyrimidin-4-amine (113d)

### Step-1: Preparation of 6-cyclopropyl-1-ethyl-1H-pyrazolo[3,4-d]pyrimidin-4-ol (113b)

Compound 113b was prepared according to the procedure reported in step-1 of scheme 1, from 6-chloro-1-ethyl-1H-pyrazolo[3,4-d]pyrimidin-4-ol (113a) (7.0 g, 35.24 mmol; CAS #1779131-19-9) in toluene (140 mL) using cyclopropylboronic acid (7b) (12.11 g, 140.97 mmol), PdCl₂(dppf)-CH₂Cl₂ adduct (1.28 g, 1.76 mmol), a solution of K₃PO₄ (29.92 g 140.97 mmol) in water (1.12 mL) and heating at 100° C. for 12 h to afford after work up and purification using flash column chromatography [silica gel, eluting with MeOH in DCM from 0-10%] 6-cyclopropyl-1-ethyl-1H-pyrazolo[3,4-d]pyrimidin-4-ol (113b) (3.0 g, 42% yield) as a brown solid; ¹H NMR (300 MHz, DMSO-d₆) δ 12.26 (s, 1H), 7.95 (s, 1H), 4.19 (q, J=7.3 Hz, 2H), 2.12-1.93 (m, 1H), 1.39-1.29 (m, 3H), 1.15-0.96 (m, 4H).

### Step-2: Preparation of 4-chloro-6-cyclopropyl-1-ethyl-1H-pyrazolo[3,4-d]pyrimidine (113c)

Compound 113c was prepared according to the procedure reported in step-3 of scheme 7, from 6-cyclopropyl-1-ethyl-1H-pyrazolo[3,4-d]pyrimidin-4-ol (113b) (1.2 g, 5.88 mmol) using POCl₃ (52.25 g, 341 mmol) and heating at 100° C. for 1 h to afford after work up and purification using flash column chromatography [silica gel, eluting with EtOAc in n-heptane from 0-50%] to afford 4-chloro-6-cyclopropyl-1-ethyl-1H-pyrazolo[3,4-d]pyrimidine (113c) (0.9 g, 69% yield) as an oily mass; ¹H NMR (300 MHz, DMSO-d₆) δ 8.29 (d, J=0.9 Hz, 1H), 4.47-4.32 (m, 2H), 2.33-2.19 (m, 1H), 1.40 (td, J=7.2, 0.9 Hz, 3H), 1.16-1.04 (m, 4H).

### Step-3: Preparation of 6-cyclopropyl-1-ethyl-N-(1-(3,4,5-trimethoxyphenyl)-1H-imidazol-4-yl)-1H-pyrazolo[3,4-d]pyrimidin-4-amine (113d)

Compound 113d was prepared according to the procedure reported in step-4 of scheme 112, from 4-chloro-6-cyclopropyl-1-ethyl-1H-pyrazolo[3,4-d]pyrimidine (113c) (3.5 g, 15.72 mmol) in 1,4-dioxane (87.5 mL) using 1-(3,4,5-trimethoxyphenyl)-1H-imidazol-4-amine (1b) (4.30 g, 17.25 mmol), cesium carbonate (15.36 g, 47.14 mmol), PdCl₂(dppf)-CH₂Cl₂ adduct (0.64 g, 0.78 mmol) and heating at 100° C. for 12 h to afford after work up and purification using flash column chromatography [silica gel, eluting with MeOH in DCM from 0-10%] 6-cyclopropyl-1-ethyl-N-(1-(3,4,5-trimethoxyphenyl)-1H-imidazol-4-yl)-1H-pyrazolo[3,4-d]pyrimidin-4-amine (113d) (3.3 g, 48% yield) free base as a grey solid; ¹H NMR (300 MHz, DMSO-d₆) δ 10.80 (s, 1H), 8.33 (s, 1H), 8.21 (s, 1H), 7.95 (s, 1H), 6.95 (s, 2H), 4.32 (q, J=7.3 Hz, 2H), 3.90 (d, J=2.2 Hz, 6H), 3.71 (d, J=2.2 Hz, 3H), 2.18-2.10 (m, 1H), 1.38 (t, J=7.3 Hz, 3H), 1.26-1.19 (m, 2H), 1.07-0.98 (m, 2H). The free base of compound 113d was converted to its HCl salt by dissolving (1.5 g, 3.44 mmol) in EtOH (15 mL), adding 14% HCl in EtOH (3 mL) and stirring for 1 h at RT to afford after work up 6-cyclopropyl-1-ethyl-N-(1-(3,4,5-trimethoxyphenyl)-1H-imidazol-4-yl)-1H-pyrazolo[3,4-d]pyrimidin-4-amine (113d) (1.45 g, 89% yield) HCl salt as a white solid; ¹H NMR (300 MHz, DMSO-d₆) δ 11.35 (s, 1H, D₂O exchangeable), 8.51 (s, 1H), 8.35 (s, 1H), 7.97 (d, J=1.7 Hz, 1H, D₂O exchangeable), 7.00 (s, 2H), 4.36 (q, J=7.2 Hz, 2H), 3.89 (s, 6H), 3.71 (s, 3H), 2.32-2.17 (m, 1H), 1.39 (t, J=7.2 Hz, 3H), 1.30-1.18 (m, 2H), 1.14-0.99 (m, 2H); MS (ES+): 436.2 (M+1); (ES−): 434.2 (M−1).

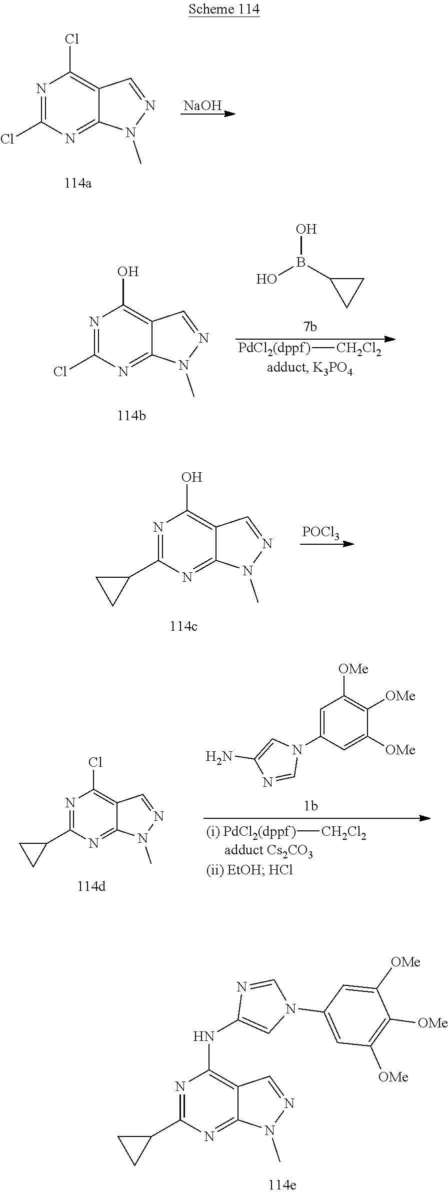

### Preparation of 6-cyclopropyl-1-methyl-N-(1-(3,4,5-trimethoxyphenyl)-1H-imidazol-4-yl)-1H-pyrazolo[3,4-d]pyrimidin-4-amine (114e)

### Step-1: Preparation of 6-chloro-1-methyl-1H-pyrazolo[3,4-d]pyrimidin-4-ol (114b)

Compound 114b was prepared according to the procedure reported in step-1 of scheme 7, from a solution of 4,6-dichloro-1-methyl-1H-pyrazolo[3,4-d]pyrimidine (114a) (22.0 g, 108.36 mmol; CAS #98141-42-5) in NaOH (2N, 135.4 mL) and heating at 90° C. for 2 h to afford after work up 6-chloro-1-methyl-1H-pyrazolo[3,4-d]pyrimidin-4-ol (114b) (20 g, 100% yield) as an oily mass; ¹H NMR (300 MHz, DMSO-d₆) δ 13.17 (s, 1H), 8.05 (s, 1H), 3.86 (s, 3H).

### Step-2: Preparation of 6-cyclopropyl-1-methyl-1H-pyrazolo[3,4-d]pyrimidin-4-ol (114c)

Compound 114c was prepared according to the procedure reported in step-1 of scheme 1, from 6-chloro-1-methyl-1H-pyrazolo[3,4-d]pyrimidin-4-ol (114b) (7.0 g, 37.92 mmol) in toluene (140 mL) using cyclopropylboronic acid (7b) (13.03 g, 151.69 mmol), PdCl₂(dppf)-CH₂Cl₂ adduct (3.09 g, 3.79 mmol), a solution of K₃PO₄ (32.0 g 151.69 mmol) in water (7 mL) and heating at 100° C. for 12 h to afford after work up and purification using flash column chromatography [silica gel, eluting with MeOH in DCM from 0-10%] 6-cyclopropyl-1-methyl-1H-pyrazolo[3,4-d]pyrimidin-4-ol (114c) (0.6 g, 8% yield) as a brown solid; ¹H NMR (300 MHz, DMSO-d₆) δ 12.26 (s, 1H), 7.94 (d, J=2.0 Hz, 1H), 3.79 (s, 3H), 2.06-2.00 (m, 1H), 1.19-0.99 (m, 4H).

### Step-3: Preparation of 4-chloro-6-cyclopropyl-1-methyl-1H-pyrazolo[3,4-d]pyrimidine (114d)

Compound 114d was prepared according to the procedure reported in step-3 of scheme 7, from 6-cyclopropyl-1-methyl-1H-pyrazolo[3,4-d]pyrimidin-4-ol (114c) (2.0 g, 10.51 mmol) using POCl₃ (93.50 g, 609.80 mmol) and heating at 100° C. for 1 h to afford after work up and purification using flash column chromatography [silica gel, eluting with 50% EtOAc in n-heptane] 4-chloro-6-cyclopropyl-1-methyl-1H-pyrazolo[3,4-d]pyrimidine (114d) (1.5 g, 68% yield) as an oily mass; ¹H NMR (300 MHz, DMSO-d₆) δ 8.31 (d, J=2.5 Hz, 1H), 4.00 (t, J=1.7 Hz, 3H), 2.29 (s, 1H), 1.18-1.08 (m, 4H).

### Step-4: Preparation of 6-cyclopropyl-1-methyl-N-(1-(3,4,5-trimethoxyphenyl)-1H-imidazol-4-yl)-1H-pyrazolo[3,4-d]pyrimidin-4-amine (114e)

Compound 114e was prepared according to the procedure reported in step-4 of scheme 112, from 4-chloro-6-cyclopropyl-1-methyl-1H-pyrazolo[3,4-d]pyrimidine (114d) (1.4 g, 6.70 mmol) in 1,4-dioxane (28 mL) using 1-(3,4,5-trimethoxyphenyl)-1H-imidazol-4-amine (1b) (2.00 g, 8.05 mmol), cesium carbonate (6.55 g 20.12 mmol), PdCl₂(dppf)-CH₂Cl₂ adduct (0.54 g, 0.67 mmol) and heating at 100° C. for 12 h to afford after work up and purification using flash column chromatography [silica gel, eluting with 10% MeOH in DCM] 6-cyclopropyl-1-methyl-N-(1-(3,4,5-trimethoxyphenyl)-1H-imidazol-4-yl)-1H-pyrazolo[3,4-d]pyrimidin-4-amine (114e) (1.5 g, 53% yield) free base as a grey solid; ¹H NMR (300 MHz, DMSO-d₆) δ 10.80 (s, 1H), 8.31 (s, 1H), 8.21 (s, 1H), 7.95 (s, 1H), 6.95 (s, 2H), 3.89 (s, 9H), 3.70 (t, J=1.7 Hz, 3H), 2.16 (s, 1H), 1.24-1.18 (m, 2H), 1.04-0.98 (m, 2H). The free base of compound 114e was converted to its HCl salt by dissolving (1.1 g, 2.60 mmol) in EtOH (16 mL), adding 14% HCl in EtOH (2.1 mL) and stirring for 1 h at RT to afford after work up 6-cyclopropyl-1-methyl-N-(1-(3,4,5-trimethoxyphenyl)-1H-imidazol-4-yl)-1H-pyrazolo[3,4-d]pyrimidin-4-amine (114e) (1.1 g, 92% yield) HCl salt as a white solid; ¹H NMR (300 MHz, DMSO-d) δ 11.33 (s, 1H, D₂O exchangeable), 8.50 (s, 1H), 8.33 (s, 1H), 7.98 (d, J=1.6 Hz, 1H, D₂O exchangeable), 7.00 (s, 2H), 3.94 (s, 3H), 3.89 (s, 6H), 2.24 (d, J=8.0, 4.1 Hz, 11-1), 1.23 (q, J=3.4 Hz, 2H), 1.09 (dd, J=7.9, 3.3 Hz, 2H); MS (ES+): 422.2 (M+1); (ES−): 420.1 (M−1); Analysis calculated for C₂₁H₂₃N₇O₃. 0.9 H₂O·1.3 HCl: C, 52.00; H, 5.42; Cl, 9.50; N, 20.21; Found: C, 52.02; H, 5.48; Cl, 9.65; N, 20.24.

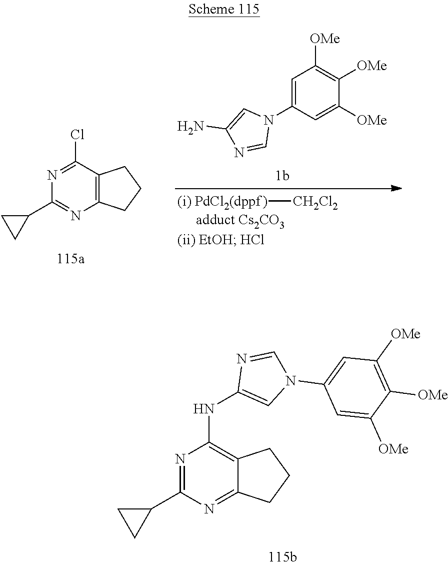

### Preparation of 2-cyclopropyl-N-(1-(3,4,5-trimethoxyphenyl)-1H-imidazol-4-yl)-6,7-dihydro-5H-cyclopenta[d]pyrimidin-4-amine (115b)

Compound 115b was prepared according to the procedure reported in step-4 of scheme 112, from 4-chloro-2-cyclopropyl-6,7-dihydro-5H-cyclopenta[d]pyrimidine (115a) (1.2 g, 6.16 mmol; CAS #1247618-11-6) in 1,4-dioxane (24 mL) using 1-(3,4,5-trimethoxyphenyl)-1H-imidazol-4-amine (1b) (1.61 g, 6.47 mmol), cesium carbonate (4.01 g, 12.32 mmol), PdCl₂(dppf)-CH₂Cl₂ adduct (0.251 g, 0.308 mmol) and heating at 90-100° C. for 12 h to afford after work up and purification using flash column chromatography [silica gel, eluting with 2-10% MeOH in DCM] 2-cyclopropyl-N-(1-(3,4,5-trimethoxyphenyl)-1H-imidazol-4-yl)-6,7-dihydro-5H-cyclopenta[d]pyrimidin-4-amine (115b) (1.1 g, 44% yield) free base as an off-white solid; ¹H NMR (400 MHz, DMSO-d₆) δ 9.28 (s, 1H), 8.09 (d, J=1.6 Hz, 1H), 7.81 (d, J=1.7 Hz, 1H), 6.85 (s, 2H), 3.82 (s, 6H), 3.63 (s, 3H), 2.77-2.64 (m, 4H), 2.07-1.98 (m, 1H), 1.96-1.87 (m, 2H), 1.05-0.97 (m, 2H), 0.92-0.83 (m, 2H). The free base of compound 115b was converted to its HCl salt by dissolving (1.0 g, 2.45 mmol) in EtOH (20 mL), adding 14% HCl in EtOH (2 mL) and stirring for 1 h at RT to afford after work up 2-cyclopropyl-N-(1-(3,4,5-trimethoxyphenyl)-1H-imidazol-4-yl)-6,7-dihydro-5H-cyclopenta[d]pyrimidin-4-amine (115b) (1.00 g, 92% yield) HCl salt as a white solid; ¹H NMR (300 MHz, DMSO-d₆) δ 11.18 (s, 1H, D₂O exchangeable), 8.33 (d, J=1.5 Hz, 1H), 7.83 (d, J=1.5 Hz, 1H), 6.96 (s, 2H), 3.89 (s, 6H), 3.71 (s, 3H), 3.04 (t, J=7.7 Hz, 2H), 2.91 (t, J=7.5 Hz, 2H), 2.41-2.12 (m, 3H), 1.48-1.23 (m, 4H); MS (ES+): 408.3 (M+1); Analysis calculated for C₂₂H₂₅N₅O₃. 2.65 H₂O·1.85 HCl: C, 50.56; H, 6.20; Cl, 12.55; N, 13.40; Found: C, 50.65; H, 6.08; Cl, 12.63; N, 13.37.

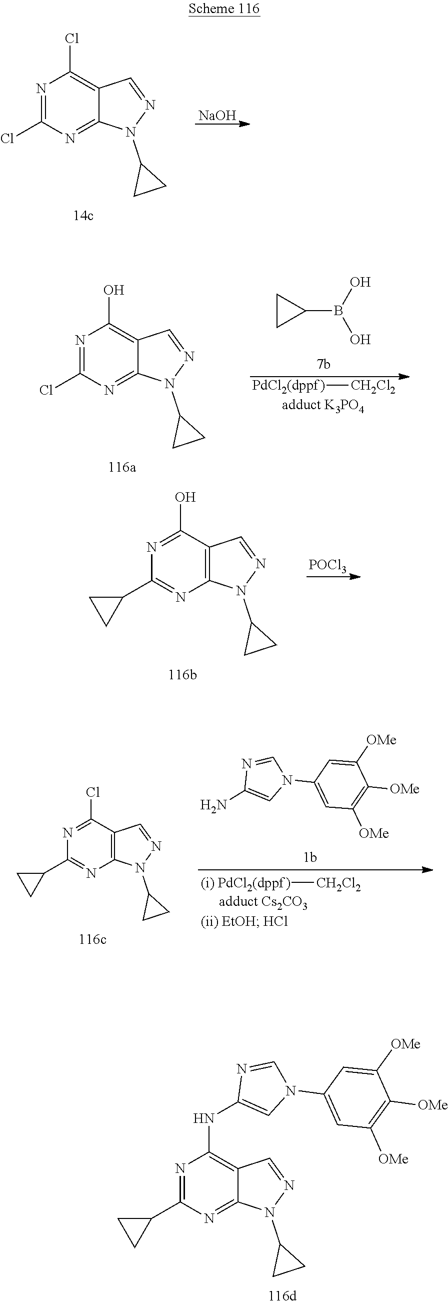

### Preparation of 1,6-dicyclopropyl-N-(1-(3,4,5-trimethoxyphenyl)-1H-imidazol-4-yl)-1H-pyrazolo[3,4-d]pyrimidin-4-amine (116d)

### Step-1: Preparation of 6-chloro-1-cyclopropyl-1H-pyrazolo[3,4-d]pyrimidin-4-ol (116a)

Compound 116a was prepared according to the procedure reported in step-1 of scheme 7, from 4,6-dichloro-1-cyclopropyl-1H-pyrazolo[3,4-d]pyrimidine (14c) (11.5 g, 50.21 mmol) in aqueous NaOH (2N, 62.5 mL) and heating at 90° C. for 1 h to afford after work up 6-chloro-1-cyclopropyl-1H-pyrazolo[3,4-d]pyrimidin-4-ol (116a) (9.0 g, 85% yield) as a yellow solid and was used as such for the next step; ¹H NMR (300 MHz, DMSO-d₆) δ 13.19 (s, 1H), 8.00 (s, 1H), 3.81-3.72 (m, 1H), 1.17-1.06 (m, 4H).

### Step-2: Preparation of 1,6-dicyclopropyl-1H-pyrazolo[3,4-d]pyrimidin-4-ol (116b)

Compound 116b was prepared according to the procedure reported in step-1 of scheme 1, from 6-chloro-1-cyclopropyl-1H-pyrazolo[3,4-d]pyrimidin-4-ol (116a) (5.5 g, 26.11 mmol) in 1,4-dioxane/toluene (110 mL; ratio 1:1) using cyclopropylboronic acid (7b) (5.60 g, 65.28 mmol), PdCl₂(dppf)-CH₂Cl₂ adduct (2.13 g, 2.61 mmol), a solution of K₃PO₄ (22.17 g, 104.45 mmol) in water (4.4 mL) and heating at 110° C. for 12 h to afford after work up and purification using flash column chromatography [silica gel, eluting with MeOH in DCM 0-3.5%] 1,6-dicyclopropyl-1H-pyrazolo[3,4-d]pyrimidin-4-ol (116b) (1.95 g, 35% yield) as an off-white solid; ¹H NMR (300 MHz, DMSO-d₆) δ 12.28 (s, 1H), 7.89 (s, 1H), 3.82-3.71 (m, 1H), 2.11-1.96 (m, 1H), 1.15-0.97 (m, 8H).

### Step-3: Preparation of 4-chloro-1,6-dicyclopropyl-1H-pyrazolo[3,4-d]pyrimidine (116c)

Compound 116c was prepared according to the procedure reported in step-3 of scheme 7, from 1,6-dicyclopropyl-1H-pyrazolo[3,4-d]pyrimidin-4-ol (116b) (2.0 g, 9.25 mmol) using POCl₃ (80.83 g, 527.19 mmol) and heating at 100° C. for 1 h to afford after work up and purification using flash column chromatography [silica gel, eluting with EtOAc in n-heptane from 0-20%] to afford 4-chloro-1,6-dicyclopropyl-1H-pyrazolo[3,4-d]pyrimidine (116c) (1.5 g, 69% yield) as an oily mass; ¹H NMR (300 MHz, DMSO-d₆) δ 8.26 (s, 1H), 3.95-3.83 (m, 1H), 2.37-2.23 (m, 1H), 1.27-1.08 (m, 8H).

### Step-4: Preparation of 1,6-dicyclopropyl-N-(1-(3,4,5-trimethoxyphenyl)-1H-imidazol-4-yl)-1H-pyrazolo[3,4-d]pyrimidin-4-amine (116d)

Compound 116d was prepared according to the procedure reported in step-4 of scheme 112, from 4-chloro-1,6-dicyclopropyl-1H-pyrazolo[3,4-d]pyrimidine (116c) (1.5 g, 6.39 mmol) in 1,4-dioxane (30 mL) using 1-(3,4,5-trimethoxyphenyl)-1H-imidazol-4-amine (1b) (1.67 g, 6.71 mmol), cesium carbonate (4.16 g, 12.78 mmol), PdCl₂(dppf)-CH₂Cl₂ adduct (0.208 g, 0.25 mmol) and heating at 100° C. for 12 h to afford after work up and purification using flash column chromatography [silica gel, eluting with MeOH in DCM from 0-3%] 1,6-dicyclopropyl-N-(1-(3,4,5-trimethoxyphenyl)-1H-imidazol-4-yl)-1H-pyrazolo[3,4-d]pyrimidin-4-amine (116d) (1.2 g, 42% yield) free base as an off-white solid; ¹H NMR (300 MHz, DMSO-d₆) δ 10.78 (s, 1H), 8.37-8.12 (m, 2H), 7.94 (s, 1H), 6.94 (s, 2H), 3.89 (s, 6H), 3.86-3.77 (m, 1H), 3.70 (s, 3H), 2.22-2.11 (m, 1H), 1.24-1.12 (m, 4H), 1.12-0.96 (m, 4H). The free base of compound 116d was converted to its HCl salt by dissolving (1.1 g, 2.46 mmol) in EtOH (22 mL), adding 14% HCl in EtOH (2.2 mL) and stirring for 1 h at RT to afford after work up 1,6-dicyclopropyl-N-(1-(3,4,5-trimethoxyphenyl)-1H-imidazol-4-yl)-1H-pyrazolo[3,4-d]pyrimidin-4-amine (116d) (1.05 g, 88% yield) HCl salt as a white solid; ¹H NMR (300 MHz, DMSO-d₆) δ 10.90 (s, 1H, D₂O exchangeable), 8.38-8.15 (m, 2H), 7.95 (d, J=1.7 Hz, 1H), 6.96 (s, 2H), 3.89 (s, 6H), 3.89-3.79 (m, 1H), 3.70 (s, 3H), 2.23-2.13 (m, 1H), 1.28-1.12 (m, 4H), 1.12-0.99 (m, 4H); MS (ES+): 448.3 (M+1); (ES−): 446.2 (M−1); Analysis calculated for C₂₃H₂₅N₇O₃. 1.25 H₂O·HCl: C, 54.54; H, 5.67; Cl, 7.00; N, 19.36; Found: C, 54.36; H, 5.33; Cl, 6.91; N, 19.22.

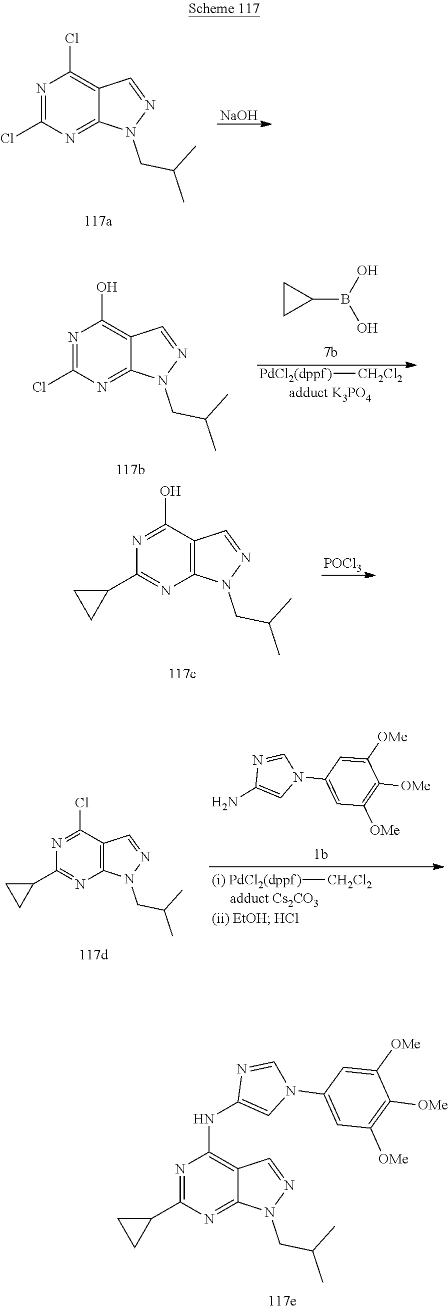

### Preparation of 6-cyclopropyl-1-isobutyl-N-(1-(3,4,5-trimethoxyphenyl)-1H-imidazol-4-yl)-1H-pyrazolo[3,4-d]pyrimidin-4-amine (117e)

### Step-1: Preparation of 6-chloro-1-isobutyl-1H-pyrazolo[3,4-d]pyrimidin-4-ol (117b)

Compound 117b was prepared according to the procedure reported in step-1 of scheme 7, from 4,6-dichloro-1-isobutyl-1H-pyrazolo[3,4-d]pyrimidine (117a) (1.35 g, 5.5 mmol; CAS #1415093-40-1) in aqueous NaOH (2N, 5.5 mL) and heating at 90° C. for 1 h to afford after work up 6-chloro-1-isobutyl-1H-pyrazolo[3,4-d]pyrimidin-4-ol (117b) (1.1 g, 88% yield) as an off-white solid, which was used as such for the next step; ¹H NMR (300 MHz, DMSO-d₆) δ 13.19 (s, 1H), 8.09 (s, 1H), 4.04 (d, J=7.2 Hz, 2H), 2.26-2.11 (m, 1H), 0.85 (d, J=6.6 Hz, 6H).

### Step-2: Preparation of 6-cyclopropyl-1-isobutyl-1H-pyrazolo[3,4-d]pyrimidin-4-ol (117c)

Compound 117c was prepared according to the procedure reported in step-1 of scheme 1, from 6-chloro-1-isobutyl-1H-pyrazolo[3,4-d]pyrimidin-4-ol (117b) (5.0 g, 22.06 mmol) in 1,4-dioxane/toluene (100 mL; ratio 1:1) using cyclopropylboronic acid (7b) (4.73 g, 55.14 mmol), PdCl₂(dppf)-CH₂Cl₂ adduct (1.8 g, 2.20 mmol), a solution of K₃PO₄ (18.73 g, 88.23 mmol) in water (3.0 mL) and heating at 110° C. for 12 h to afford after work up and purification using flash column chromatography [silica gel, eluting with MeOH in DCM from 0-3.5%] 6-cyclopropyl-1-isobutyl-1H-pyrazolo[3,4d]pyrimidin-4-ol (117c) (3.0 g, 59% yield) as an off-white solid; ¹H NMR (300 MHz, DMSO-d₆) δ 12.27 (s, 1H), 7.96 (s, 1H), 3.98 (d, J=7.0 Hz, 2H), 2.23-2.07 (m, 1H), 2.07-1.97 (m, 1H), 1.07 (d, J=6.2 Hz, 4H), 0.81 (d, J=6.7 Hz, 6H).

### Step-3: Preparation of 4-chloro-6-cyclopropyl-1-isobutyl-1H-pyrazolo[3,4-d]pyrimidine (117d)

Compound 117d was prepared according to the procedure reported in step-3 of scheme 7, from 6-cyclopropyl-1-isobutyl-1H-pyrazolo[3,4-d]pyrimidin-4-ol (117c) (2.0 g, 8.61 mmol) using POCl₃ (75.24 g, 490.76 mmol) and heating at 100° C. for 1 h to afford after work up and purification using flash column chromatography [silica gel, eluting with EtOAc in n-heptane from 0-20%] 4-chloro-6-cyclopropyl-1-isobutyl-1H-pyrazolo[3,4-d]pyrimidine (117d) (2.0 g, 93% yield) as an oily mass; ¹H NMR (300 MHz, DMSO-d₆) δ 8.33 (d, J=1.6 Hz, 1H), 4.21 (d, J=7.2 Hz, 2H), 2.36-2.16 (m, 2H), 1.21-1.07 (m, 4H), 0.85 (d, J=6.6 Hz, 6H).

### Step-4: Preparation of 6-cyclopropyl-1-isobutyl-N-(1-(3,4,5-trimethoxyphenyl)-1H-imidazol-4-yl)-1H-pyrazolo[3,4-d]pyrimidin-4-amine (117e)

Compound 117e was prepared according to the procedure reported in step-4 of scheme 112, from 4-chloro-6-cyclopropyl-1-isobutyl-1H-pyrazolo[3,4-d]pyrimidine (117d) (1.9 g, 7.58 mmol) in 1,4-dioxane (38 mL) using 1-(3,4,5-trimethoxyphenyl)-1H-imidazol-4-amine (1b) (1.98 g, 7.95 mmol), cesium carbonate (4.93 g, 15.15 mmol), PdCl₂(dppf)-CH₂Cl₂ adduct (0.309 g, 0.378 mmol) and heating at 90-100° C. for 12 h to afford after work up and purification using flash column chromatography [silica gel, eluting with MeOH in DCM from 0-3%] 6-cyclopropyl-1-isobutyl-N-(1-(3,4,5-trimethoxyphenyl)-1H-imidazol-4-yl)-1H-pyrazolo[3,4-d]pyrimidin-4-amine (117e) (1.6 g, 46% yield) free base as an off-white solid; ¹H NMR (400 MHz, DMSO-d₆) δ 10.81 (s, 1H), 8.33 (s, 1H), 8.21 (d, J=1.7 Hz, 1H), 7.94 (d, J=1.8 Hz, 1H), 6.94 (s, 2H), 4.10 (d, J=7.2 Hz, 2H), 3.88 (s, 6H), 3.69 (s, 3H), 2.31-2.17 (m, 1H), 2.17-2.08 (m, 1H), 1.25-1.17 (m, 2H), 1.06-0.97 (m, 2H), 0.84 (d, J=6.8 Hz, 6H); MS (ES+): 464.4 (M+1); (ES−): 462.2 (M−1). The free base of compound 117e was converted to its HCl salt by dissolving (1.2 g, 2.59 mmol) in EtOH (24 mL), adding 14% HCl in EtOH (2 mL) and stirring for 1 h at RT to afford after work up 6-cyclopropyl-1-isobutyl-N-(1-(3,4,5-trimethoxyphenyl)-1H-imidazol-4-yl)-1H-pyrazolo[3,4-d]pyrimidin-4-amine (117e) (1.1 g, 85% yield) HCl salt as a white solid; ¹H NMR (300 MHz, DMSO-d₆) δ 11.11 (s, 1H, D₂O exchangeable), 8.50-8.22 (m, 2H), 7.97 (d, J=1.7 Hz, 1H, D₂O exchangeable), 6.98 (s, 2H), 4.14 (s, 2H), 3.89 (s, 6H), 3.70 (s, 3H), 2.34-2.09 (m, 2H), 1.29-1.14 (m, 2H), 1.13-0.98 (m, 2H), 0.85 (d, J=6.7 Hz, 6H); MS (ES+): 464.3 (M+1); (ES−): 462.1 (M−1); Analysis calculated for C₂₄H₂₉N₇O₃·H₂O·1.25HCl: C, 54.68; H, 6.17; Cl, 8.41; N, 18.60; Found: C, 54.75; H, 6.32; Cl, 8.01; N, 18.66.

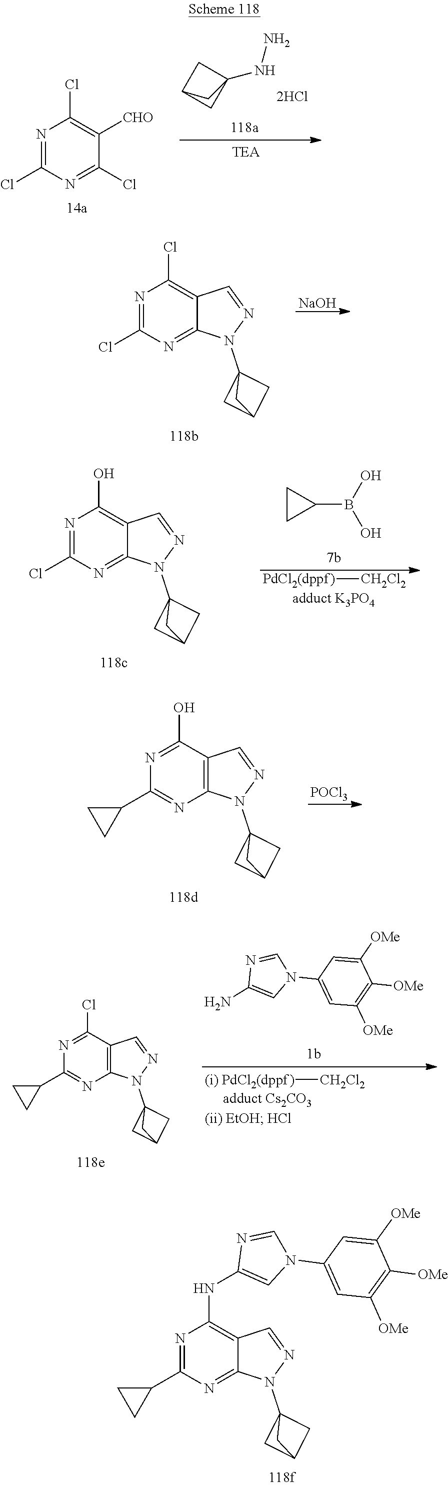

### Preparation of 1-(bicyclo[1.1.1]pentan-1-yl)-6-cyclopropyl-N-(1-(3,4,5-trimethoxyphenyl)-1H-imidazol-4-yl)-1H-pyrazolo[3,4-d]pyrimidin-4-amine (118f

### Step-1: Preparation of 1-(bicyclo[1.1.1]pentan-1-yl)-4,6-dichloro-1H-pyrazolo[3,4-d]pyrimidine (118b)

Compound 118b was prepared according to the procedure reported in step-1 of scheme 14, from 2,4,6-trichloropyrimidine-5-carbaldehyde (14a) (4.94 g, 23.36 mmol) in EtOH (100 mL) using bicyclo[1.1.1]pentan-1-ylhydrazine dihydrochloride (118a) (3.99 g, 23.32 mmol; CAS #1403746-38-2) in EtOH (40 mL), triethylamine (9.46 g, 93.52 mmol) and stirring at −78° C. for 2 h followed by stirring in ice water bath for 1 h (the reaction mixture was poured into ice water) to afford after work up 1-(bicyclo[1.1.1]pentan-1-yl)-4,6-dichloro-1H-pyrazolo[3,4-d]pyrimidine (118b) (4.5 g, 76% yield) as an off-white solid; ¹H NMR (300 MHz, DMSO-d₆) δ 8.54 (s, 1H), 2.75 (s, 1H), 2.45 (s, 6H).

### Step-2: Preparation of 1-(bicyclo[1.1.1]pentan-1-yl)-6-chloro-1H-pyrazolo[3,4-d]pyrimidin-4-ol (118c)

Compound 118c was prepared according to the procedure reported in step-1 of scheme 7, from 1-(bicyclo[1.1.1]pentan-1-yl)-4,6-dichloro-1H-pyrazolo[3,4-d]pyrimidine (118b) (4.5 g, 17.64 mmol) in aqueous NaOH (2N, 22 mL) and heating at 90° C. for 1 h to afford after work up 1-(bicyclo[1.1.1]pentan-1-yl)-6-chloro-1H-pyrazolo[3,4-d]pyrimidin-4-ol (118c) (3.9 g, 94% yield) as a yellow solid and was used as such for the next step; ¹H NMR (300 MHz, DMSO-d₆) δ 13.24 (s, 1H), 8.07 (s, 1H), 2.67 (s, 1H), 2.37 (s, 6H).

### Step-3: Preparation of 1-(bicyclo[1.1.1]pentan-1-yl)-6-cyclopropyl-1H-pyrazolo[3,4-d]pyrimidin-4-ol (118d)

Compound 118d was prepared according to the procedure reported in step-1 of scheme 1, from 1-(bicyclo[1.1.1]pentan-1-yl)-6-chloro-1H-pyrazolo[3,4-d]pyrimidin-4-ol (118c) (1.8 g, 7.6 mmol) in 1,4-dioxane/toluene (36 mL; ratio 1:1) using cyclopropylboronic acid (7b) (1.63 g, 18.97 mmol), PdCl₂(dppf)-CH₂Cl₂ adduct (0.62 g, 0.76 mmol), a solution of K₃PO₄ (6.45 g, 30.42 mmol) in water (1.6 mL) and heating at 110° C. for 12 h to afford after work up and purification using flash column chromatography [silica gel, eluting with MeOH in DCM from 0-3.5%] 1-(bicyclo[1.1.1]pentan-1-yl)-6-cyclopropyl-1H-pyrazolo[3,4-d]pyrimidin-4-ol (118d) (1.4 g, 76% yield) as an off-white solid; ¹H NMR (300 MHz, DMSO-d₆) δ 12.32 (s, 1H), 7.92 (s, 1H), 2.63 (s, 1H), 2.33 (s, 6H), 2.08-1.96 (m, 1H), 1.07 (d, J=6.1 Hz, 4H).

### Step-4: Preparation of 1-(bicyclo[1.1.1]pentan-1-yl)-4-chloro-6-cyclopropyl-1H-pyrazolo[3,4-d]pyrimidine (118e)

Compound 118e was prepared according to the procedure reported in step-3 of scheme 7, from 1-(bicyclo[1.1.1]pentan-1-yl)-6-cyclopropyl-1H-pyrazolo[3,4-d]pyrimidin-4-ol (118d) (2.2 g, 9.08 mmol) using POCl₃ (79.36 g, 517.58 mmol) and heating at 100° C. for 1 h to afford after work up and purification using flash column chromatography [silica gel, eluting with EtOAc in n-heptane from 0-20%] 1-(bicyclo[1.1.1]pentan-1-yl)-4-chloro-6-cyclopropyl-1H-pyrazolo[3,4-d]pyrimidine (118e) (1.7 g, 72% yield) as an oily mass; ¹H NMR (300 MHz, DMSO-d₆) δ 8.32 (d, J=2.2 Hz, 1H), 2.73 (s, 1H), 2.44 (d, J=1.7 Hz, 6H), 2.27 (s, 1H), 1.20-1.05 (m, 4H).

### Step-5: Preparation of 1-(bicyclo[1.1.1]pentan-1-yl)-6-cyclopropyl-N-(1-(3,4,5-trimethoxyphenyl)-1H-imidazol-4-yl)-1H-pyrazolo[3,4-d]pyrimidin-4-amine (118f)

Compound 118f was prepared according to the procedure reported in step-4 of scheme 112, from 1-(bicyclo[1.1.1]pentan-1-yl)-4-chloro-6-cyclopropyl-1H-pyrazolo[3,4-d]pyrimidine (118e) (1.7 g, 6.52 mmol) in 1,4-dioxane (37 mL) using 1-(3,4,5-trimethoxyphenyl)-1H-imidazol-4-amine (1b) (1.7 g, 6.84 mmol), cesium carbonate (4.24 g, 13.04 mmol), PdCl₂(dppf)-CH₂Cl₂ adduct (0.26 g, 0.32 mmol) and heating at 90° C. for 12 h to afford after work up and purification using flash column chromatography [silica gel, eluting with MeOH in DCM from 0-3%] 1-(bicyclo[1.1.1]pentan-1-yl)-6-cyclopropyl-N-(1-(3,4,5-trimethoxyphenyl)-1H-imidazol-4-yl)-1H-pyrazolo[3,4-d]pyrimidin-4-amine (118f) (1.1 g, 36% yield) free base as an off-white solid; ¹H NMR (300 MHz, DMSO-d₆) δ 10.79 (s, 1H), 8.32 (s, 1H), 8.20 (s, 1H), 7.95 (s, 1H), 6.94 (s, 2H), 3.89 (s, 6H), 3.70 (s, 3H), 2.67 (s, 1H), 2.41 (s, 6H), 2.17-2.11 (m, 1H), 1.23-1.17 (m, 2H), 1.07-1.00 (m, 2H). The free base of compound 118f was converted to its HCl salt by dissolving (1.05 g, 2.21 mmol) in EtOH (20 mL), adding 14% HCl in EtOH (17%) (2 mL) and stirring for 1 h at RT to afford after work up 1-(bicyclo[1.1.1]pentan-1-yl)-6-cyclopropyl-N-(1-(3,4,5-trimethoxyphenyl)-1H-imidazol-4-yl)-1H-pyrazolo[3,4-d]pyrimidin-4-amine (118f) (1.05 g, 93% yield) HCl salt as a white solid; ¹H NMR (300 MHz, DMSO-d₆) δ 10.98 (s, 1H, D₂O exchangeable), 8.41 (s, 1H), 8.29 (s, 1H), 7.98 (d, J=1.6 Hz, 1H), 6.98 (s, 2H), 3.89 (s, 6H), 3.70 (s, 3H), 2.67 (s, 1H), 2.40 (s, 6H), 2.23-2.10 (m, 1H), 1.23-1.12 (m, 2H), 1.10-0.96 (m, 2H); MS (ES+): 474.3 (M+1); (ES−): 472.2 (M−1); Analysis calculated for C₂₅H₂₇N₇O₃·1.25H₂O·1.5HCl: C, 54.52; H, 5.67; Cl, 9.66; N, 17.80; Found: C, 54.75; H, 5.58; Cl, 9.62; N, 17.86.

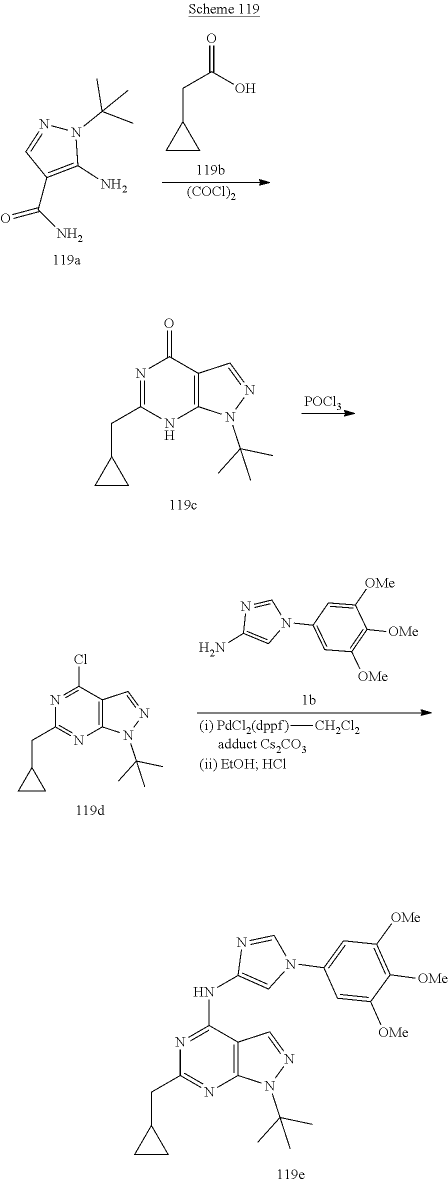

### Preparation of 1-(tert-butyl)-6-(cyclopropylmethyl)-N-(1-(3.4,5-trimethoxyphenyl)-1H-imidazol-4-yl)-1H-pyrazolo[3,4-d]pyrimidin-4-amine (119e)

### Step-1: Preparation of 1-(tert-butyl)-6-(cyclopropylmethyl)-1H-pyrazolo[3,4-d]pyrimidin-4 (7H)-one (119c)

Compound 119c was prepared according to the procedure reported in step-1 of scheme 27, using 2-cyclopropylacetic acid (119b) (5.0 g, 49.94 mmol) in DCM (100 mL) and oxalyl chloride (19.0 g, 149.82 mmol), DMF (5 drops) and stirring at RT for 1.5 h to afford after work up 2-cyclopropylacetyl chloride (6 g). To a solution of 5-amino-1-(tert-butyl)-1H-pyrazole-4-carboxamide (119a) (5.0 g, 27.44 mmol) in 1,4-dioxane (100 mL) was added 2-cyclopropylacetyl chloride (6 g) in 1,4-dioxane (60 mL) at RT and stirring at RT for 12 h to afford after work up and purification using column chromatography [silica gel, eluting with MeOH in DCM from 0-5%] 1-(tert-butyl)-6-(cyclopropylmethyl)-1H-pyrazolo[3,4-d]pyrimidin-4 (7H)-one (119c) (2 g, 30% yield) as an oily mass, ¹H NMR (300 MHz, DMSO-d₆) δ 12.00 (s, 1H), 7.92 (d, J=2.2 Hz, 1H), 2.24-2.08 (m, 2H), 1.69 (s, 9H), 0.99-0.82 (m, 1H), 0.26 (d, J=5.0 Hz, 2H), 0.10 (d, J=4.9 Hz, 2H).

### Step-2: Preparation of 1-(tert-butyl)-4-chloro-6-(cyclopropylmethyl)-1H-pyrazolo[3,4-d]pyrimidine (119d)

Compound 119d was prepared according to the procedure reported in step-3 of scheme 7, from 1-(tert-butyl)-6-(cyclopropylmethyl)-1H-pyrazolo[3,4-d]pyrimidin-4 (7H)-one (119c) (2.5 g, 10.15 mmol) using POCl₁ h (90.26 g, 588.68 mmol) and heating at 100° C. for 1 h to afford after work up and purification using flash column chromatography [silica gel, eluting with MeOH in DCM from 0-10%] 1-(tert-butyl)-4-chloro-6-(cyclopropylmethyl)-1H-pyrazolo[3,4-d]pyrimidine (119d) (1.5 g, 56% yield) as an oily mass; ¹H NMR (300 MHz, DMSO-d₆) δ 8.02 (s, 1H), 2.58 (d, J=7.0 Hz, 2H), 1.49 (s, 9H), 0.61-0.51 (m, 1H), 0.30-0.17 (m, 2H), 0.02-−0.10 (m, 2H).

### Step-3: Preparation of 1-(tert-butyl)-6-(cyclopropylmethyl)-N-(1-(3,4,5-trimethoxyphenyl)-1H-imidazol-4-yl)-1H-pyrazolo[3,4-d]pyrimidin-4-amine (119e)

Compound 119e was prepared according to the procedure reported in step-4 of scheme 7, from 1-(tert-butyl)-4-chloro-6-(cyclopropylmethyl)-1H-pyrazolo[3,4-d]pyrimidine (119d) (1.5 g, 5.67 mmol) in 1,4-dioxane (30 mL) using 1-(3,4,5-trimethoxyphenyl)-1H-imidazol-4-amine (1b) (1.69 g, 6.78 mmol), cesium carbonate (3.69 g, 11.33 mmol), PdCl₂(dppf)-CH₂Cl₂ adduct (0.46 g, 0.56 mmol) and heating at 100° C. for 14 h to afford after work up and purification using flash column chromatography [silica gel, eluting with MeOH in DCM from 0-10%] 1-(tert-butyl)-6-(cyclopropylmethyl)-N-(1-(3,4,5-trimethoxyphenyl)-1H-imidazol-4-yl)-1H-pyrazolo[3,4-d]pyrimidin-4-amine (119e) (900 mg, 33% yield) free base as a white solid; ¹H NMR (300 MHz, DMSO-d₆) δ 10.73 (s, 1H), 8.35 (s, 1H), 8.15 (d, J=16.8 Hz, 2H), 6.92 (s, 2H), 3.87 (s, 6H), 3.70 (s, 3H), 2.75 (d, J=6.9 Hz, 2H), 1.73 (s, 9H), 1.23 (s, 1H), 0.56-0.46 (m, 2H), 0.35-0.27 (m, 2H). The free base of compound 119e was converted to its HCl salt by dissolving (600 mg, 1.26 mmol) in EtOH (18 mL), adding 14% HCl in EtOH (1.8 mL) and stirring for 1 h at RT to afford after work up 1-(tert-butyl)-6-(cyclopropylmethyl)-N-(1-(3,4,5-trimethoxyphenyl)-1H-imidazol-4-yl)-1H-pyrazolo[3,4-d]pyrimidin-4-amine (119e) (550 mg, 85% yield) HCl salt as a white solid; ¹H NMR (300 MHz, DMSO-d₆) δ 11.30 (s, 1H, D₂O exchangeable), 8.39 (s, 2H), 8.06 (d, J=1.6 Hz, 1H), 6.97 (s, 2H), 3.88 (s, 6H), 3.70 (s, 3H), 2.80 (d, J=6.9 Hz, 2H), 1.74 (s, 9H), 1.26 (dq, J=8.0, 5.2 Hz, 1H), 0.64-0.49 (m, 2H), 0.41-0.24 (m, 2H); MS (ES+): 478.2 (M+1); (ES−): 476.2 (M−1); Analysis calculated for C₂₅H₃₁N₇O₃·2H₂O·HCl: C, 54.59; H, 6.60; Cl, 6.45; N, 17.83; Found: C, 54.44; H, 6.52; Cl, 6.25; N, 17.78.

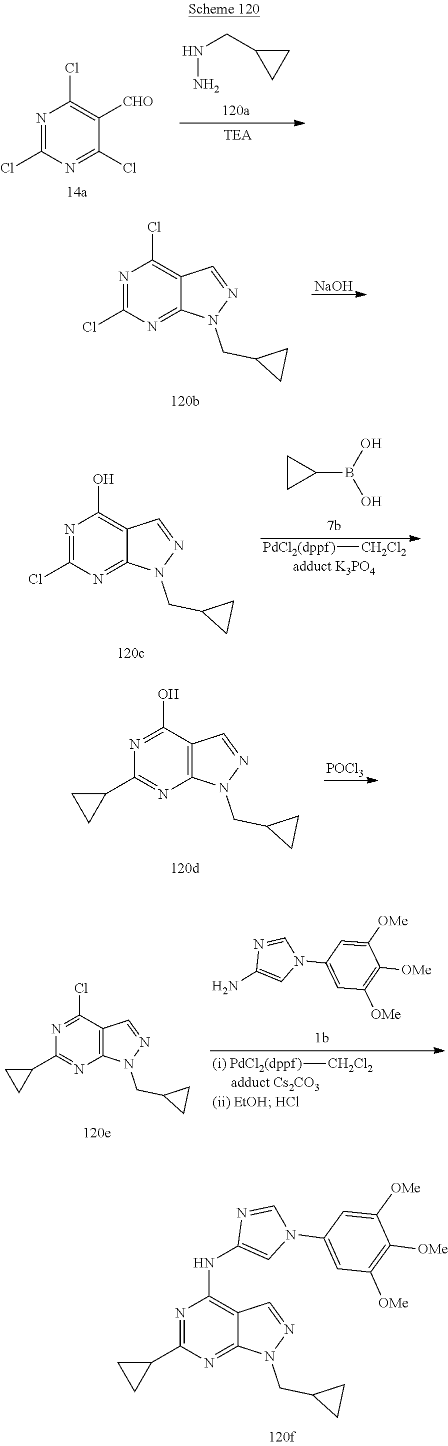

### Preparation of 6-cyclopropyl-1-(cyclopropylmethyl)-N-(1-(3,4,5-trimethoxyphenyl)-1H-imidazol-4-yl)-1H-pyrazolo[3,4-d]pyrimidin-4-amine (120)

### Step-1: Preparation of 4,6-dichloro-1-(cyclopropylmethyl)-1H-pyrazolo[3,4-d]pyrimidine (120b)

Compound 120b was prepared according to the procedure reported in step-1 of scheme 14, from 2,4,6-trichloropyrimidine-5-carbaldehyde (14a) (76.0 g, 359.46 mmol) in EtOH (1146 mL) using (cyclopropylmethyl)hydrazine (120a) (31.0 g, 359.45 mmol; CAS #40487-93-2) in EtOH (380 mL), triethylamine (100.2 mL, 718.9 mmol) and stirring at −78° C. to RT for 2 h to afford after work up and purification using flash column chromatography [silica gel, eluting with EtOAc in n-heptane from 10-100%] 4,6-dichloro-1-(cyclopropylmethyl)-1H-pyrazolo[3,4-d]pyrimidine (120b) (46.2 g, 53% yield) as a green liquid; ¹H NMR (300 MHz, DMSO-d₆) δ 8.64-8.49 (m, 1H), 4.30 (d, J=7.1 Hz, 2H), 1.22-1.11 (m, 1H), 0.60-0.50 (m, 2H), 0.46-0.40 (m, 2H).

### Step-2: Preparation of 6-chloro-1-(cyclopropylmethyl)-1H-pyrazolo[3,4-d]pyrimidin-4-ol (120c)

Compound 120c was prepared according to the procedure reported in step-1 of scheme 7, from 4,6-dichloro-1-(cyclopropylmethyl)-1H-pyrazolo[3,4-d]pyrimidine (120b) (30.0 g, 123.41 mmol) in aqueous NaOH (2N, 300 mL) and heating at 90° C. for 2 h to afford after work up 6-chloro-1-(cyclopropylmethyl)-1H-pyrazolo[3,4-d]pyrimidin-4-ol (120c) (21.9 g, 79% yield) as a white solid; ¹H NMR (300 MHz, DMSO-d₆) δ 13.19 (s, 1H), 8.09 (d, J=2.1 Hz, 1H), 4.10 (d, J=7.0 Hz, 2H), 1.36-1.19 (m, 1H), 0.56-0.47 (m, 2H), 0.40-0.33 (m, 2H).

### Step-3: Preparation of 6-cyclopropyl-1-(cyclopropylmethyl)-1H-pyrazolo[3,4-d]pyrimidin-4-ol (120d)

Compound 120d was prepared according to the procedure reported in step-1 of scheme 1, from 6-chloro-1-(cyclopropylmethyl)-1H-pyrazolo[3,4-d]pyrimidin-4-ol (120c) (10.0 g, 44.51 mmol) in toluene (332 mL) using cyclopropylboronic acid (7b) (7.64 g, 89.03 mmol). PdCl₂(dppf)-CH₂Cl₂ adduct (3.63 g, 4.45 mmol), a solution of K₃PO₄ (37.79 g, 178.04 mmol) in water (22 mL) and heating at 110° C. for 14 h to afford after work up and triturating with methanol, 6-cyclopropyl-1-(cyclopropylmethyl)-1H-pyrazolo[3,4-d]pyrimidin-4-ol (120d) (3.9 g, 38% yield) as a yellow solid; ¹H NMR (300 MHz, DMSO-d₆) δ 12.27 (s, 1H), 7.95 (s, 1H), 4.03 (d, J=7.1 Hz, 2H), 2.10-1.96 (m, 1H), 1.28-1.15 (m, 1H), 1.07 (d, J=6.3 Hz, 4H), 0.54-0.43 (m, 2H), 0.41-0.32 (m, 2H).

### Step-4: Preparation of 4-chloro-6-cyclopropyl-1-(cyclopropylmethyl)-1H-pyrazolo[3,4-d]pyrimidine (120e)

Compound 120e was prepared according to the procedure reported in step-3 of scheme 7, from 6-cyclopropyl-1-(cyclopropylmethyl)-1H-pyrazolo[3,4-d]pyrimidin-4-ol (120d) (3.2 g, 13.90 mmol) using POCl₃ (64 mL) and heating at 100° C. for 2 h to afford after work up and purification using flash column chromatography [silica gel, eluting with EtOAc in n-heptane from 0-10%] 4-chloro-6-cyclopropyl-1-(cyclopropylmethyl)-1H-pyrazolo[3,4-d]pyrimidine (120e) (3.2 g, 92% yield) as an off-white solid; ¹H NMR (300 MHz, DMSO-d₆) δ 8.47-8.11 (m, 1H), 4.25 (d, J=7.0 Hz, 2H), 2.36-2.18 (m, 1H), 1.19-1.06 (m, 4H), 0.95-0.78 (m, 1H), 0.62-0.46 (m, 2H), 0.46-0.36 (m, 2H).

### Step-5: Preparation of 6-cyclopropyl-1-(cyclopropylmethyl)-N-(1-(3,4,5-trimethoxyphenyl)-1H-imidazol-4-yl)-1H-pyrazolo[3,4-d]pyrimidin-4-amine (120f)

Compound 120f was prepared according to the procedure reported in step-4 of scheme 112, from 4-chloro-6-cyclopropyl-1-(cyclopropylmethyl)-1H-pyrazolo[3,4-d]pyrimidine (120e) (3.28 g, 13.19 mmol) in 1,4-dioxane (65.6 mL) using 1-(3,4,5-trimethoxyphenyl)-1H-imidazol-4-amine (1b) (3.94 g, 15.83 mmol), cesium carbonate (8.59 g, 26.38 mmol), PdCl₂(dppf)-CH₂Cl₂ adduct (0.538 g, 0.66 mmol) and heating at 100° C. for 14 h to afford after work up and triturating with methanol, 6-cyclopropyl-1-(cyclopropylmethyl)-N-(1-(3,4,5-trimethoxyphenyl)-1H-imidazol-4-yl)-1H-pyrazolo[3,4-d]pyrimidin-4-amine (120f) (2.6 g, 43% yield) free base as an off-white solid; ¹H NMR (300 MHz, DMSO-d₆) δ 10.80 (s, 1H), 8.33 (s, 1H), 8.21 (s, 1H), 7.95 (s, 1H), 6.95 (s, 2H), 4.16 (d, J=6.8 Hz, 2H), 3.89 (s, 6H), 3.70 (s, 3H), 2.15 (s, 1H), 1.28 (s, 1H), 1.21 (s, 2H), 1.06-0.97 (m, 2H), 0.52-0.36 (m, 4H). The free base of compound 120f was converted to its HCl salt by dissolving (1.5 g, 3.25 mmol) in EtOH (7.5 mL), adding 14% HCl in EtOH (3 mL) and stirring for 1 h at RT to afford after work up 6-cyclopropyl-1-(cyclopropylmethyl)-N-(1-(3,4,5-trimethoxyphenyl)-1H-imidazol-4-yl)-1H-pyrazolo[3,4-d]pyrimidin-4-amine (120f) (1.4 g, 86% yield) HCl salt as an off-white solid; ¹H NMR (300 MHz, DMSO-d₆) δ 11.20 (s, 1H), 8.46 (s, 1H), 8.34 (s, 1H), 7.98 (d, J=1.7 Hz, 1H), 6.99 (s, 2H), 4.20 (d, J=7.0 Hz, 2H), 3.89 (s, 6H), 3.71 (s, 3H), 2.32-2.13 (m, 1H), 1.37-1.16 (m, 3H), 1.12-1.01 (m, 2H), 0.54-0.45 (m, 2H), 0.45-0.36 (m, 2H); MS (ES+): 462.3 (M+1); (ES−): 460.2 (M−1); Analysis calculated for C₂₄H₂₇N₇O₃·1.75H₂O·HCl: C, 54.44; H, 6.00; Cl, 6.69; N, 18.52; Found: C, 54.44; H, 5.85; Cl, 6.74; N, 18.45.

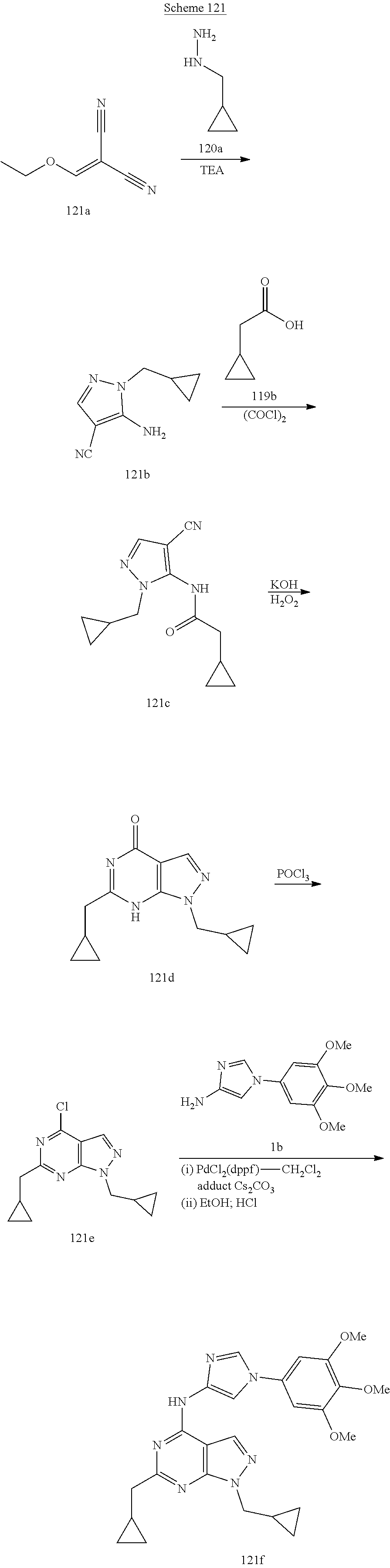

### Preparation of 1,6-bis(cyclopropylmethyl)-N-(1-(3,4,5-trimethoxyphenyl)-1H-imidazol-4-yl)-1H-pyrazolo[3,4-d]pyrimidin-4-amine (121f)

### Step-1: Preparation of 5-amino-1-(cyclopropylmethyl)-1H-pyrazole-4-carbonitrile (121b)

To a stirred solution of 2-(ethoxymethylene)malononitrile (121a) (38.0 g, 311.14 mmol; CAS #123-06-8) in EtOH (760.0 mL) was added triethylamine (31.48 g, 311.14 mmol) drop wise, (cyclopropylmethyl)hydrazine (120a) (26.80 g, 311.14 mmol) at RT and was stirred at 60° C. for 1 h. The reaction mixture was concentrated and the residue obtained was purified using column chromatography [silica gel, eluting with EtOAc] to give 5-amino-1-(cyclopropylmethyl)-1H-pyrazole-4-carbonitrile (121b) (14.0 g, 28% yield) as an off-white solid; ¹H NMR (300 MHz, DMSO-d₆) δ 7.52 (s, 1H), 6.54 (s, 2H), 3.77 (d, J=6.9 Hz, 2H), 1.23-1.04 (m, 1H), 0.51-0.36 (m, 2H), 0.32 (dt, J=5.1, 2.8 Hz, 2H).

### Step-2: Preparation of N-(4-cyano-1-(cyclopropylmethyl)-1H-pyrazol-5-yl)-2-cyclopropylacetamide (121c)

Compound 121c was prepared according to the procedure reported in step-1 of scheme 27, using 2-cyclopropylacetic acid (119b) (4.5 g, 44.95 mmol) in DCM (90 mL), oxalyl chloride (17.11 g, 134.83 mmol), DMF (5 drops) and stirring at RT for 4.5 h to afford after work up 2-cyclopropylacetyl chloride (5.5 g). To a solution of 5-amino-1-(cyclopropylmethyl)-1H-pyrazole-4-carbonitrile (121b) (3.0 g, 18.50 mmol) in 1,4-dioxane (300 mL) was added 2-cyclopropylacetyl chloride (5.5 g) in 1,4-dioxane (55 mL) at RT and stirred at 60° C. for 12 h. This afforded after work up N-(4-cyano-1-(cyclopropylmethyl)-1H-pyrazol-5-yl)-2-cyclopropylacetamide (121c) (4 g, 88% yield) and was used as such for the next step.

### Step-3: Preparation of 1,6-bis(cyclopropylmethyl)-1,7-dihydro-4H-pyrazolo[3,4-d]pyrimidin-4-one (121d)

To a stirred solution of N-(4-cyano-1-(cyclopropylmethyl)-1H-pyrazol-5-yl)-2-cyclopropylacetamide (121c) (3.5 g, 14.33 mmol) in aqueous KOH (5N, 34.4 mL) was added H₂O₂ (50% in water, 70.0 mL) drop wise at RT and stirred at 85° C. for 2 h. The reaction mixture was cooled to RT, pH was adjusted to acidic by using 1N HCl, and extracted with ethyl acetate (2×500 mL). The organic layer was washed with brine, dried, filtered and concentrated to give 1,6-bis(cyclopropylmethyl)-1,7-dihydro-4H-pyrazolo[3,4-d]pyrimidin-4-one (121d) (2.5 g, 71% yield) as an off-white solid; ¹H NMR (300 MHz, DMSO-d₆) δ 12.03 (s, 1H), 8.00 (s, 1H), 4.12 (d, J=7.0 Hz, 2H), 2.52 (s, 2H), 1.34-1.06 (m, 2H), 0.55-0.32 (m, 6H), 0.32-0.22 (m, 2H).

### Step-4: Preparation of 4-chloro-1,6-bis(cyclopropylmethyl)-1H-pyrazolo[3,4-d]pyrimidine (121e)

Compound 121e was prepared according to the procedure reported in step-3 of scheme 7, from 1,6-bis(cyclopropylmethyl)-1,7-dihydro-4H-pyrazolo[3,4-d]pyrimidin-4-one (121d) (2.0 g, 8.19 mmol) using POCl₃ (71.54 g, 466.63 mmol) and heating at 100° C. for 1 h to afford after work up and purification using flash column chromatography [silica gel, eluting with MeOH in DCM from 0-10%] 4-chloro-1,6-bis(cyclopropylmethyl)-1H-pyrazolo[3,4-d]pyrimidine (121e) (1.65 g, 77% yield) as an oily mass; ¹H NMR (300 MHz, DMSO-d₆) δ 8.14 (s, 1H), 4.05 (d, J=7.1 Hz, 2H), 2.59 (d, J=7.1 Hz, 2H), 1.14-0.88 (m, 2H), 0.35-0.13 (m, 6H), 0.07-−0.21 (m, 2H).

### Step-5: Preparation of 1,6-bis(cyclopropylmethyl)-N-(1-(3,4,5-trimethoxyphenyl)-1H-imidazol-4-yl)-1H-pyrazolo[3,4-d]pyrimidin-4-amine (121f)

Compound 121f was prepared according to the procedure reported in step-4 of scheme 112, from 4-chloro-1,6-bis(cyclopropylmethyl)-1H-pyrazolo[3,4-d]pyrimidine (121e) (1.65 g, 6.28 mmol) in 1,4-dioxane (33 mL) using 1-(3,4,5-trimethoxyphenyl)-1H-imidazol-4-amine (1b) (1.64 g, 6.59 mmol), cesium carbonate (4.069 g, 12.55 mmol), PdCl₂ (dppf)-CH₂Cl₂ adduct (0.256 g, 0.313 mmol) and heating at 100° C. for 4 h to afford after work up and purification using flash column chromatography [silica gel, eluting with MeOH in DCM from 0-10%]1,6-bis(cyclopropylmethyl)-N-(1-(3,4,5-trimethoxyphenyl)-1H-imidazol-4-yl)-1H-pyrazolo[3,4-d]pyrimidin-4-amine (1210 (1.1 g, 37% yield) free base as an off-white solid; ¹H NMR (300 MHz, DMSO-d) δ 10.85 (s, 1H), 8.39 (s, 1H), 8.17 (d, J=12.7 Hz, 2H), 6.92 (s, 2H), 4.17 (d, J=7.0 Hz, 2H), 3.87 (s, 6H), 3.69 (s, 3H), 2.75 (d, J=7.0 Hz, 2H), 1.44-1.12 (m, 2H), 0.56-0.44 (m, 4H), 0.43-0.37 (m, 2H), 0.34-0.28 (m, 2H). The free base of compound 121f was converted to its HCl salt by dissolving (1.0 g, 2.10 mmol) in EtOH (20 mL), adding 14% HCl in EtOH (3 mL) and stirring for 1 h at RT to afford after work up 1,6-bis(cyclopropylmethyl)-N-(1-(3,4,5-trimethoxyphenyl)-1H-imidazol-4-yl)-1H-pyrazolo[3,4-d]pyrimidin-4-amine (121f) (0.7 g, 65% yield) HCl salt as a white solid; ¹H NMR (300 MHz, DMSO-d₆) δ 11.79 (s, 1H, D₂O exchangeable), 8.47 (s, 2H), 8.09 (d, J=1.6 Hz, 1H), 6.99 (s, 2H), 4.23 (d, J=7.0 Hz, 2H), 3.89 (s, 6H), 3.71 (s, 3H), 2.83 (d, J=7.0 Hz, 2H), 1.38-1.18 (m, 2H), 0.65-0.55 (m, 2H), 0.55-0.47 (m, 2H), 0.47-0.41 (m, 2H), 0.41-0.32 (m, 2H); MS (ES+): 476.3 (M+1); (ES−): 474.2 (M−1); Analysis calculated for C₂₅H₂₉N₇O₃·1.1H₂O·1.1HCl: C, 56.08; H, 6.08; Cl, 7.28; N, 18.31; Found: C, 56.04; H, 5.98; Cl, 7.48; N, 18.18.

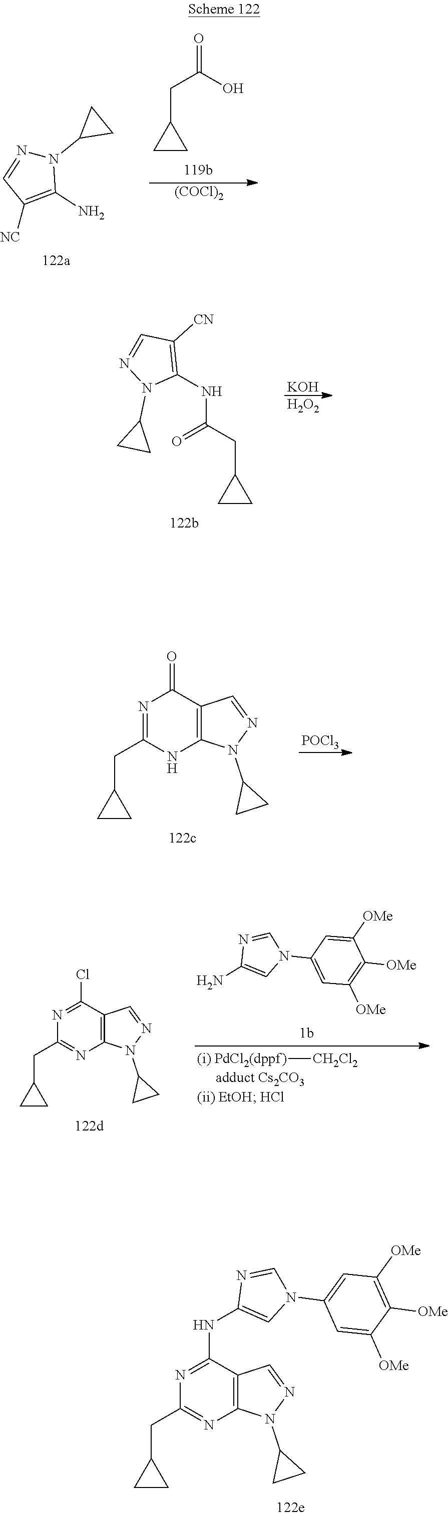

### Preparation of 1-cyclopropyl-6-(cyclopropylmethyl)-N-(1-(3,4,5-trimethoxyphenyl)-1H-imidazol-4-yl)-1H-pyrazolo[3,4-d]pyrimidin-4-amine (122e)

### Step-1: Preparation of N-(4-cyano-1-cyclopropyl-1H-pyrazol-5-yl)-2-cyclopropylacetamide (122b)

Compound 122b was prepared according to the procedure reported in step-1 of scheme 27, using 2-cyclopropylacetic acid (119b) (4.1 g, 40.95 mmol) in DCM (82 mL), oxalyl chloride (10.53 g, 83.01 mmol), DMF (5 drops) and stirring at RT for 1.5 h to afford after work up 2-cyclopropylacetyl chloride (4.8 g). To a solution of 5-amino-1-cyclopropyl-1H-pyrazole-4-carbonitrile (122a) (3.0 g, 20.25 mmol) in 1,4-dioxane (60 mL) was added 2-cyclopropylacetyl chloride (4.8 g) in 1,4-dioxane (15 mL) at RT stirred at 60° C. for 12 h to afford after work up N-(4-cyano-1-cyclopropyl-1H-pyrazol-5-yl)-2-cyclopropylacetamide (122b) (4.66 g, 100% yield) as an off-white solid, which was used as such for the next step.

### Step-2: Preparation of 1-cyclopropyl-6-(cyclopropylmethyl)-1H-pyrazolo[3,4-d]pyrimidin-4 (7H)-one (122c)

Compound 122c was prepared according to the procedure reported in step-3 of scheme 121, from N-(4-cyano-1-cyclopropyl-1H-pyrazol-5-yl)-2-cyclopropylacetamide (122b) (4.66 g, 20.24 mmol) in aqueous KOH (5N, 48.5 mL) using H₂O₂ (30% in water, 93.2 mL) and stirring at 85° C. for 2 h to afford after work up and purification 1-cyclopropyl-6-(cyclopropylmethyl)-1H-pyrazolo[3,4-d]pyrimidin-4 (7H)-one (122c) (2.5 g, 54% yield) as a white solid; ¹H NMR (300 MHz, DMSO-d₆) δ 12.04 (s, 1H), 7.94 (s, 1H), 3.92-3.79 (m, 1H), 2.52 (d, J=7.7 Hz, 2H), 1.27-1.04 (m, 5H), 0.55-0.43 (m, 2H), 0.34-0.23 (m, 2H).

### Step-3: Preparation of 4-chloro-1-cyclopropyl-6-(cyclopropylmethyl)-1H-pyrazolo[3,4-d]pyrimidine (122d)

Compound 122d was prepared according to the procedure reported in step-3 of scheme 7, from 1-cyclopropyl-6-(cyclopropylmethyl)-1H-pyrazolo[3,4-d]pyrimidin-4 (7H)-one (122c) (2.0 g, 8.69 mmol) using POCl₃ (71.51 g, 466.40 mmol) and heating at 100° C. for 1 h to afford after work up and purification using flash column chromatography [silica gel, eluting with EtOAc in n-heptane from 0-20%] 4-chloro-1-cyclopropyl-6-(cyclopropylmethyl)-1H-pyrazolo[3,4-d]pyrimidine (122d) (1.7 g, 79% yield) as an oily mass; ¹H NMR (300 MHz, DMSO-d₆) δ 8.30 (s, 1H), 4.00-3.87 (m, 1H), 2.84 (d, J=7.0 Hz, 2H), 1.25-1.08 (m, 5H), 0.55-0.41 (m, 2H), 0.32-0.21 (m, 2H).

### Step-4: Preparation of 1-cyclopropyl-6-(cyclopropylmethyl)-N-(1-(3,4,5-trimethoxyphenyl)-1H-imidazol-4-yl)-1H-pyrazolo[3,4-d]pyrimidin-4-amine (122e)

Compound 122e was prepared according to the procedure reported in step-4 of scheme 112, from 4-chloro-1-cyclopropyl-6-(cyclopropylmethyl)-1H-pyrazolo[3,4-d]pyrimidine (122d) (1.7 g, 6.84 mmol) in 1,4-dioxane (34 mL) using 1-(3,4,5-trimethoxyphenyl)-1H-imidazol-4-amine (1b) (1.78 g, 7.17 mmol), cesium carbonate (4.45 g, 13.67 mmol), PdCl₂(dppf)-CH₂Cl₂ adduct (0.27 g, 0.34 mmol) and heating at 100° C. for 12 h to afford after work up and purification using flash column chromatography [silica gel, eluting with MeOH in DCM from 0-10%] 1-cyclopropyl-6-(cyclopropylmethyl)-N-(1-(3,4,5-trimethoxyphenyl)-1H-imidazol-4-yl)-1H-pyrazolo[3,4-d]pyrimidin-4-amine (122e) (1.0 g, 32% yield) free base as an off-white solid; ¹H NMR (300 MHz, DMSO-d₆) δ 10.83 (s, 1H), 8.33 (s, 1H), 8.17 (d, J=12.0 Hz, 2H), 6.92 (s, 2H), 3.87 (s, 6H), 3.86-3.79 (m, 1H), 3.70 (s, 3H), 2.77 (d, J=6.9 Hz, 2H), 1.29 (s, 1H), 1.22-1.01 (m, 4H), 0.58-0.46 (m, 2H), 0.43-0.25 (m, 2H). The free base of compound 122e was converted to its HCl salt by dissolving (1.0 g, 2.17 mmol) in EtOH (20 mL), adding 14% HCl in EtOH (3 mL) and stirring for 1 h at RT to afford after work up 1-cyclopropyl-6-(cyclopropylmethyl)-N-(1-(3,4,5-trimethoxyphenyl)-1H-imidazol-4-yl)-1H-pyrazolo[3,4-d]pyrimidin-4-amine (122e) (0.95 g, 88% yield) HCl salt as a white solid; ¹H NMR (300 MHz, DMSO-d₆) δ 12.05 (s, 1H, D₂O exchangeable), 8.87-8.19 (m, 2H), 8.06 (d, J=1.6 Hz, 1H), 7.00 (s, 2H), 3.98-3.89 (m, 1H), 3.88 (s, 6H), 3.70 (s, 3H), 2.86 (d, J=7.0 Hz, 2H), 1.35-1.01 (m, 5H), 0.69-0.51 (m, 2H), 0.46-0.27 (m, 2H); MS (ES+): 462.3 (M+1); (ES−): 460.2 (M−1); Analysis calculated for C₂₄H₂₇N₇O₁.1.25H₂O·1.25 HCl: C, 54.43; H, 5.85; Cl, 8.37; N, 18.51; Found: C, 54.30; H, 5.85; Cl, 8.39; N, 18.39.

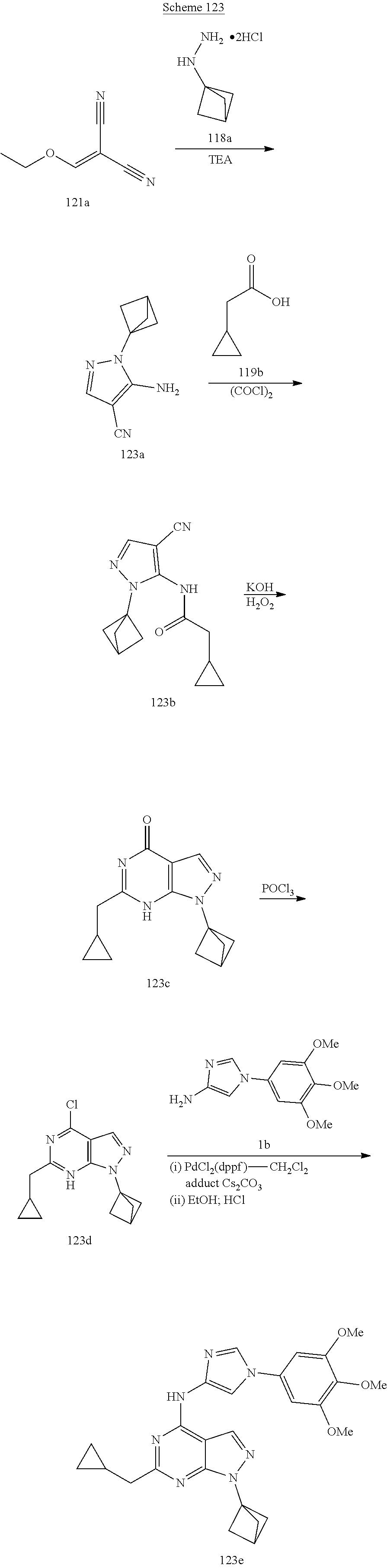

### Preparation of 1-(bicyclo[1.1.1]pentan-1-yl)-6-(cyclopropylmethyl)-N-(1-(3,4,5-trimethoxyphenyl)-1H-imidazol-4-yl)-1H-pyrazolo[3,4-d]pyrimidin-4-amine (123e)

### Step-1: Preparation of 5-amino-1-(bicyclo[1.1.1]pentan-1-yl)-1H-pyrazole-4-carbonitrile (123a)

Compound 123a was prepared according to the procedure reported in step-1 of scheme 121, from 2-(ethoxymethylene)malononitrile (121a) (7.13 g, 58.38 mmol) in EtOH (200 mL) using triethylamine (11.83 g, 116.91 mmol) and bicyclo[1.1.1]pentan-1-ylhydrazine dihydrochloride (118a) (10.0 g, 58.45 mmol; CAS #1403746-38-2) to afford after work up and purification using column chromatography [silica gel, eluting with EtOAc from 0-45%]5-amino-1-(bicyclo[1.1.1]pentan-1-yl)-1H-pyrazole-4-carbonitrile (123a) (6.0 g, 59% yield) as an off-white solid; ¹H NMR (300 MHz. DMSO-d₆) δ 7.50 (s, 1H), 6.40 (s, 2H), 2.55 (s, 1H), 2.29 (s, 6H).

### Step-2: Preparation of N-(1-(bicyclo[1.1.1]pentan-1-yl)-4-cyano-H-pyrazol-5-yl)-2-cyclopropylacetamide (123b)

Compound 123b was prepared according to the procedure reported in step-1 of scheme 27, using 2-cyclopropylacetic acid (119b) (3.5 g, 34.96 mmol) in DCM (70 mL) and oxalyl chloride (13.31 g, 104.87 mmol), DMF (5 drops) and stirring at RT for 4.5 h to afford after work up 2-cyclopropylacetyl chloride (4.13 g). To a solution of 5-amino-1-(bicyclo[1.1.1]pentan-1-yl)-1H-pyrazole-4-carbonitrile (123a) (3.0 g, 17.22 mmol) in 1,4-dioxane (90 mL) was added 2-cyclopropylacetyl chloride (4.13 g) in 1,4-dioxane (50 mL) at RT and heated at 60° C. for 12 h to afford after work up N-(1-(bicyclo[1.1.1]pentan-1-yl)-4-cyano-1H-pyrazol-5-yl)-2-cyclopropylacetamide (123b) (4.2 g), which was used as such for the next step; MS (ES+): 257.3 (M+1); (ES−): 255.2 (M−1).

### Step-3: Preparation of 1-(bicyclo[1.1.1]pentan-1-yl)-6-(cyclopropylmethyl)-1,7-dihydro-4H-pyrazolo[3,4-d]pyrimidin-4-one (123c)

Compound 123c was prepared according to the procedure reported in step-3 of scheme 121, from N-(1-(bicyclo[1.1.1]pentan-1-yl)-4-cyano-1H-pyrazol-5-yl)-2-cyclopropylacetamide (123b) (3.0 g, 11.70 mmol) in KOH (5N) (28.1 mL) using H₂O₂ (50% in water) (60 mL) and stirring at 85° C. for 2 h to afford after work up and purification 1-(bicyclo[1.1.1]pentan-1-yl)-6-(cyclopropylmethyl)-1,7-dihydro-4H-pyrazolo[3,4-d]pyrimidin-4-one (123c) (2.4 g, 80% yield) as an off-white solid; ¹H NMR (300 MHz, DMSO-d₆) δ 12.05 (s, 1H), 7.97 (s, 1H), 2.66 (s, 1H), 2.52 (s, 2H), 2.38 (s, 6H), 1.14 (dp, J=10.9, 3.9, 2.9 Hz, 1H), 0.56-0.44 (m, 2H), 0.32-0.21 (m, 2H).

### Step-4: Preparation of 1-(bicyclo[1.1.1]pentan-1-yl)-4-chloro-6-(cyclopropylmethyl)-1H-pyrazolo[3,4-d]pyrimidine (123d)

Compound 123d was prepared according to the procedure reported in step-3 of scheme 7, from 1-(bicyclo[1.1.1]pentan-1-yl)-6-(cyclopropylmethyl)-1,7-dihydro-4H-pyrazolo[3,4-d]pyrimidin-4-one (123c) (2.0 g, 7.80 mmol) using POCl₃ (68.19 g, 444.77 mmol) and heating at 100° C. for 1 h to afford after work up and purification using flash column chromatography [silica gel, eluting with EtOAc in n-heptane from 0-30%] 1-(bicyclo[1.1.1]pentan-1-yl)-4-chloro-6-(cyclopropylmethyl)-1H-pyrazolo[3,4-d]pyrimidine (123d) (1.5 g, 70% yield) as an oily mass; ¹H NMR (300 MHz, DMSO-d₆) δ 8.37 (s, 1H), 2.84 (d, J=7.0 Hz, 2H), 2.72 (s, 1H), 2.45 (s, 6H), 1.32-1.13 (m, 1H), 0.57-0.42 (m, 2H), 0.37-0.18 (m, 2H).

### Step-5: Preparation of 1-(bicyclo[1.1.1]pentan-1-yl)-6-(cyclopropylmethyl)-N-(1-(3,4,5-trimethoxyphenyl)-1H-imidazol-4-yl)-1H-pyrazolo[3,4-d]pyrimidin-4-amine (123e)

Compound 123e was prepared according to the procedure reported in step-4 of scheme 112, from 1-(bicyclo[1.1.1]pentan-1-yl)-4-chloro-6-(cyclopropylmethyl)-1H-pyrazolo[3,4-d]pyrimidine (123d) (1.4 g, 5.10 mmol) in 1,4-dioxane (28 mL) using 1-(3,4,5-trimethoxyphenyl)-1H-imidazol-4-amine (1b) (1.64 g, 6.58 mmol), cesium carbonate (3.32 g, 10.19 mmol), PdCl₂(dppf)-CH₂Cl₂ adduct (0.208 g, 0.254 mmol) and heating at 100° C. for 4 h to afford after work up and purification using flash column chromatography [silica gel, eluting with MeOH in DCM from 0-10%] 1-(bicyclo[1.1.1]pentan-1-yl)-6-(cyclopropylmethyl)-N-(1-(3,4,5-trimethoxyphenyl)-1H-imidazol-4-yl)-1H-pyrazolo[3,4-d]pyrimidin-4-amine (123e) (1.1 g, 44% yield) free base as an off-white solid; ¹H NMR (300 MHz, DMSO-d₆) δ 10.82 (s, 1H), 8.38 (s, 1H), 8.16 (d, J=14.6 Hz, 2H), 6.92 (s, 2H), 3.87 (s, 6H), 3.70 (s, 3H), 2.75 (d, J=6.9 Hz, 2H), 2.67 (s, 11H), 2.41 (s, 6H), 1.52-1.12 (m, 11H), 0.64-0.46 (m, 2H), 0.46-0.25 (m, 2H). The free base of compound 123e was converted to its HCl salt by dissolving (1.0 g, 2.05 mmol) in EtOH (20 mL), adding 14% HCl in EtOH (3 mL) and stirring for 1 h at RT to afford after work up 1-(bicyclo[1.1.1]pentan-1-yl)-6-(cyclopropylmethyl)-N-(1-(3,4,5-trimethoxyphenyl)-1H-imidazol-4-yl)-1H-pyrazolo[3,4-d]pyrimidin-4-amine (123e) (0.7 g, 65% yield) HCl salt as a white solid; ¹H NMR (300 MHz, DMSO-d₆) δ 11.64 (s, 11H, D₂O exchangeable), 8.72-8.24 (m, 2H), 8.08 (d, J=1.6 Hz, 11H), 6.99 (s, 2H), 3.89 (s, 6H), 3.71 (s, 3H), 2.81 (d, J=7.0 Hz, 2H), 2.70 (s, 1H), 2.43 (s, 6H), 1.36-1.17 (m, 114), 0.65-0.53 (m, 2H), 0.40-0.32 (m, 2H); MS (ES+): 488.3 (M+1); (ES−): 486.2 (M−1); Analysis calculated for C₂₆H₂₉N₇O₃. 1.25 H₂O·HCl: C, 57.14; H, 5.99; Cl, 6.49; N, 17.94; Found: C, 56.88; H, 5.97; Cl, 6.75; N, 17.82.

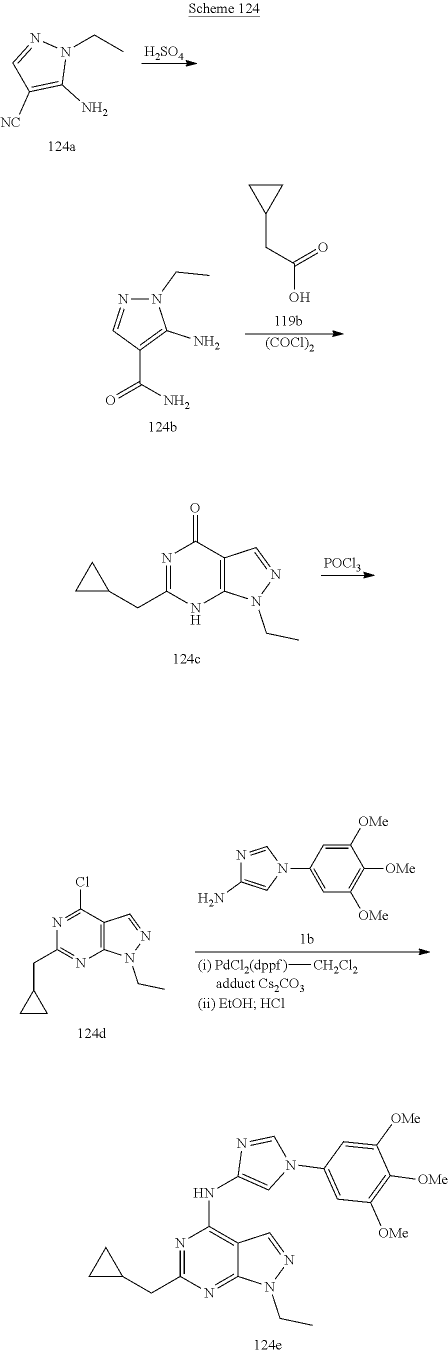

### Preparation of 6-(cyclopropylmethyl)-1-ethyl-N-(1-(3,4,5-trimethoxyphenyl)-1H-imidazol-4-yl)-1H-pyrazolo[3,4-d]pyrimidin-4-amine (124e)

### Step-1: Preparation of 5-amino-1-ethyl-H-pyrazole-4-carboxamide (124b)

To Conc. H₂SO₄ solution (25.0 mL) was added portion wise at 25° C.-50° C. 5-amino-1-ethyl-1H-pyrazole-4-carbonitrile (124a) (10.0 g, 73.44 mmol; CAS #4788-15-2) and the mixture was stirred at RT for 1 h. The reaction mixture was cooled and pH was adjusted to neutral with 3N NaOH solution (100 mL) and the resulted solid was filtered and dried in oven at 60° C. to give 5-amino-1-ethyl-1H-pyrazole-4-carboxamide (124b) (6.0 g, 53% yield) as an off-white solid; ¹H NMR (300 MHz. DMSO-d₆) δ 7.62 (s, 1H), 7.16 (s, 1H), 6.65 (s, 1H), 6.16 (s, 2H), 3.87 (q, J=7.2 Hz, 2H), 1.20 (t, J=7.1 Hz, 3H).

### Step-2: Preparation of 6-(cyclopropylmethyl)-1-ethyl-1,7-dihydro-4H-pyrazolo[3,4-d]pyrimidin-4-one (124c)

Compound 124c was prepared according to the procedure reported in step-1 of scheme 27, using 2-cyclopropylacetic acid (119b) (4 g, 39.95 mmol) in DCM (80 mL), oxalyl chloride (15.21 g, 119.85 mmol), DMF (0.5 mL) and stirring at RT for 1.5 h to afford after work up 2-cyclopropylacetyl chloride (4.6 g). To a solution of 5-amino-1-ethyl-1H-pyrazole-4-carboxamide (124b) (3.0 g, 19.46 mmol,) in 1,4-dioxane (15 mL) was added 2-cyclopropylacetyl chloride (4.6 g) in 1,4-dioxane (15 mL) at RT and stirred at 60° C. for 16 h to afford after work up 6-(cyclopropylmethyl)-1-ethyl-1,7-dihydro-4H-pyrazolo[3,4-d]pyrimidin-4-one (124c) (1.8 g crude, 42% yield) as an off-white solid, which was used as such for the next step.

### Step-3: Preparation of 4-chloro-6-(cyclopropylmethyl)-1-ethyl-1H-pyrazolo[3,4-d]pyrimidine (124d)

Compound 124d was prepared according to the procedure reported in step-3 of scheme 7, from 6-(cyclopropylmethyl)-1-ethyl-1,7-dihydro-4H-pyrazolo[3,4-d]pyrimidin-4-one (124c) (2.0 g, 9.16 mmol) using POCl₃ (81.49 g, 531.47 mmol) and heating at 100° C. for 1 h to afford after work up and purification using flash column chromatography [silica gel, eluting with EtOAc in n-heptane from 0-30%] 4-chloro-6-(cyclopropylmethyl)-1-ethyl-1H-pyrazolo[3,4-d]pyrimidine (124d) (1.7 g, 78% yield) as an oily mass; ¹H NMR (300 MHz, DMSO-d₆) δ 8.11 (s, 1H), 4.20 (q, J=7.2 Hz, 2H), 2.58 (d, J=7.0 Hz, 2H), 1.18 (t, J=7.2 Hz, 3H), 0.58 (t, J=6.6 Hz, 1H), 0.30-0.15 (m, 2H), 0.07-−0.05 (m, 2H).

### Step-4: Preparation of 6-(cyclopropylmethyl)-1-ethyl-N-(1-(3,4,5-trimethoxyphenyl)-1H-imidazol-4-yl)-1H-pyrazolo[3,4-d]pyrimidin-4-amine (124e)

Compound 124e was prepared according to the procedure reported in step-4 of scheme 112, from 4-chloro-6-(cyclopropylmethyl)-1-ethyl-1H-pyrazolo[3,4-d]pyrimidine (124d) (2.0 g, 8.45 mmol) in 1,4-dioxane (40 mL) using 1-(3,4,5-trimethoxyphenyl)-1H-imidazol-4-amine (1b) (2.21 g, 8.87 mmol), cesium carbonate (5.50 g, 16.89 mmol), PdCl₂(dppf)-CH₂Cl₂ adduct (0.34 g, 0.42 mmol) and heating at 100° C. for 16 h to afford after work up, purification [silica gel, eluting with MeOH in DCM from 0-10%] followed by trituration with MeOH, filtration and drying 6-(cyclopropylmethyl)-1-ethyl-N-(1-(3,4,5-trimethoxyphenyl)-1H-imidazol-4-yl)-1H-pyrazolo[3,4-d]pyrimidin-4-amine (124e) (1.7 g, 45% yield) free base as an off-white solid; ¹H NMR (300 MHz, DMSO-d₆) δ 10.84 (s, 1H), 8.39 (s, 1H), 8.17 (d, J=11.9 Hz, 2H), 6.92 (s, 2H), 4.32 (d, J=8.9 Hz, 2H), 3.87 (s, 6H), 3.70 (s, 3H), 2.75 (d, J=6.5 Hz, 2H), 1.56-1.10 (m, 4H), 0.52 (s, 2H), 0.32 (s, 2H). The free base of compound 124e was repurified using reverse phase column chromatography [C18 (50 g), eluting with ACN in water (containing 0.1% HCl) from 0-55%] to afford 6-(cyclopropylmethyl)-1-ethyl-N-(1-(3,4,5-trimethoxyphenyl)-1H-imidazol-4-yl)-1H-pyrazolo[3,4-d]pyrimidin-4-amine (124e) (925 mg, 50% yield) HCl salt as a white solid; ¹H NMR (300 MHz, DMSO-d₆) δ 11.68 (s, 1H, D₂O exchangeable), 8.62-8.30 (m, 2H), 8.08 (d, J=1.6 Hz, 1H), 6.98 (s, 2H), 4.37 (q, J=7.2 Hz, 2H), 3.88 (s, 6H), 3.70 (s, 3H), 2.82 (d, J=7.0 Hz, 2H), 1.40 (t, J=7.2 Hz, 3H), 1.36-1.18 (m, 1H), 0.64-0.49 (m, 2H), 0.42-0.27 (m, 2H); MS (ES+): 450.3 (M+1); (ES−): 448.2 (M−1); Analysis calculated for C₂₃H₂₇N-03.1.25H₂O·HCl: C, 54.33; H, 6.05; Cl, 6.97; N, 19.28; Found: C, 54.42; H, 6.12; Cl, 6.89; N, 19.20.

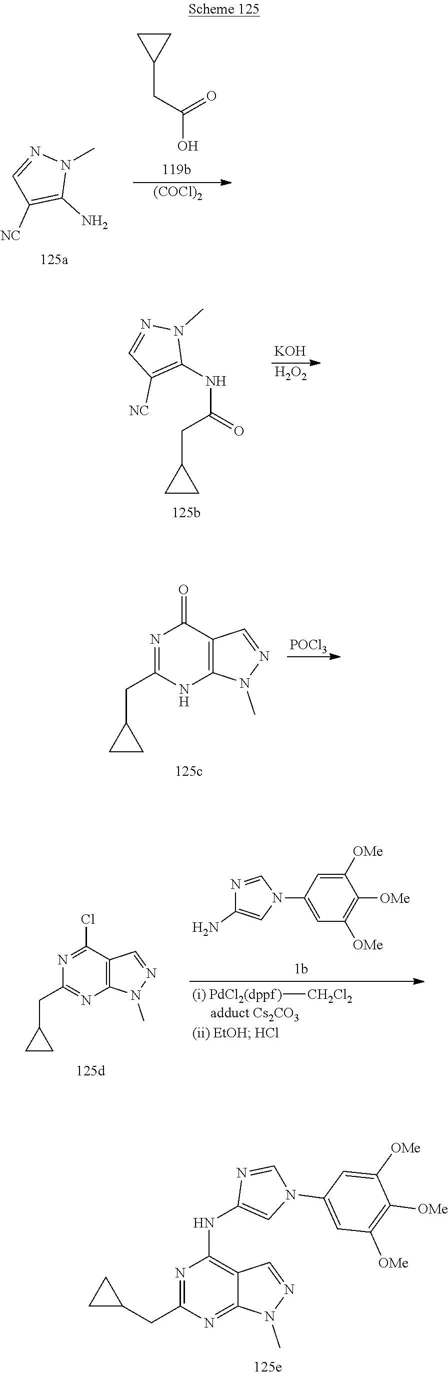

### Preparation of 6-(cyclopropylmethyl)-1-methyl-N-(1-(3,4,5-trimethoxyphenyl)-1H-imidazol-4-yl)-1H-pyrazolo[3,4-d]pyrimidin-4-amine (125e)

### Step-1: Preparation of N-(4-cyano-1-methyl-1H-pyrazol-5-yl)-2-cyclopropylacetamide (125b)

Compound 125b was prepared according to the procedure reported in step-1 of scheme 27, using 2-cyclopropylacetic acid (119b) (6.0 g, 59.93 mmol) in DCM (120 mL), oxalyl chloride (22.81 g, 179.78 mmol), DMF (0.5 mL) and stirring at 0° C. to RT for 1.5 h to afford after work up 2-cyclopropylacetyl chloride (7.1 g). To a solution of 5-amino-1-methyl-1H-J0 pyrazole-4-carbonitrile (125a) (3.64 g, 29.80 mmol; CAS #5334-41-8) in 1,4-dioxane (120 mL) was added 2-cyclopropylacetyl chloride (6 g) in 1,4-dioxane (30 mL) at RT and stirred at 60° C. for 12 h to afford after work up N-(4-cyano-1-methyl-1H-pyrazol-5-yl)-2-cyclopropylacetamide (125b) (6.0 g, 99% yield) as an off-white solid, which was used as such for the next step.

### Step-2: Preparation of 6-(cyclopropylmethyl)-1-methyl-1,7-dihydro-4H-pyrazolo[3,4-d]pyrimidin-4-one (125c)

Compound 125c was prepared according to the procedure reported in step-3 of scheme 121, from N-(4-cyano-1-methyl-1H-pyrazol-5-yl)-2-cyclopropylacetamide (125b) (3.2 g, 15.67 mmol) in aqueous KOH (5N, 37.6 mL) using H₂O₂ (30% in water, 64.0 mL) and stirring at 85° C. for 2 h. The reaction mixture was extracted with 20% MeOH in DCM (2×250 mL), washed with brine, dried, filtered and concentrated to get crude product, which was triturated with n-heptane (50 mL), and filtered to afford 6-(cyclopropylmethyl)-1-methyl-1,7-dihydro-4H-pyrazolo[3,4-d]pyrimidin-4-one (125c) (1.2 g, 38% yield) as white solid; ¹H NMR (300 MHz, DMSO-d₆) δ 12.03 (s, 1H), 7.99 (s, 1H), 3.88 (s, 3H), 2.53 (s, 2H), 1.22-1.09 (m, 1H), 0.55-0.43 (m, 2H), 0.38-0.22 (m, 2H).

### Step-3: Preparation of 4-chloro-6-(cyclopropylmethyl)-1-methyl-1H-pyrazolo[3,4-d]pyrimidine (125d)

Compound 125d was prepared according to the procedure reported in step-3 of scheme 7, from 6-(cyclopropylmethyl)-1-methyl-1,7-dihydro-4H-pyrazolo[3,4-d]pyrimidin-4-one (125c) (1.2 g, 5.88 mmol) using POCl₃ 3 (52.25 g, 340.79 mmol) and heating to 100° C. for 1 h to afford after work up and purification using flash column chromatography [silica gel, eluting with EtOAc in n-heptane from 0-20%] 4-chloro-6-(cyclopropylmethyl)-1-methyl-1H-pyrazolo[3,4-d]pyrimidine (125d) (1.1 g, 84% yield) as a white solid; ¹H NMR (300 MHz, DMSO-d₆) δ 8.37 (s, 1H), 4.04 (s, 3H), 2.84 (d, J=7.0 Hz, 2H), 1.33-1.14 (m, 1H), 0.58-0.42 (m, 2H), 0.32-0.20 (m, 2H).

### Step-4: Preparation of 6-(cyclopropylmethyl)-1-methyl-N-(1(3,4,5-trimethoxyphenyl)-1H-imidazol-4-yl)-1H-pyrazolo[3,4-d]pyrimidin-4-amine (125e)

Compound 125e was prepared according to the procedure reported in step-4 of scheme 7, from 4-chloro-6-(cyclopropylmethyl)-1-methyl-1H-pyrazolo[3,4-d]pyrimidine (125d) (1.1 g, 4.94 mmol) in 1,4-dioxane (22 mL) using 1-(3,4,5-trimethoxyphenyl)-1H-imidazol-4-amine (1b) (1.29 g, 5.17 mmol), cesium carbonate (3.21 g, 9.87 mmol), PdCl₂(dppf)-CH₂Cl₂ adduct (0.20 g, 0.24 mmol) and heating at 100° C. for 16 h to afford after work up and purification using flash column chromatography [silica gel, eluting with MeOH in DCM from 0-10%] 6-(cyclopropylmethyl)-1-methyl-N-(1-(3,4,5-trimethoxyphenyl)-1H-imidazol-4-yl)-1H-pyrazolo[3,4-d]pyrimidin-4-amine (125e) (1.0 g, 47% yield) free base as an off-white solid. ¹H NMR (300 MHz, DMSO-d₆) δ 10.85 (s, 1H), 8.37 (s, 1H), 8.17 (d, J=11.1 Hz, 2H), 6.92 (s, 2H), 3.91 (s, 3H), 3.87 (s, 6H), 3.69 (s, 3H), 2.76 (d, J=7.0 Hz, 2H), 1.69-0.89 (m, 1H), 0.57-0.48 (m, 2H), 0.36-0.28 (m, 2H). The free base of compound 125e was converted to its HCl salt by dissolving (1.0 g, 2.30 mmol) in EtOH (20 mL), adding 14% HCl in EtOH (3 mL) and stirring for 1 h at RT to afford after work up 6-(cyclopropylmethyl)-1-methyl-N-(1-(3,4,5-trimethoxyphenyl)-1H-imidazol-4-yl)-1H-pyrazolo[3,4-d]pyrimidin-4-amine (125e) (1.05 g, 97% yield) HCl salt as a white solid; ¹H NMR (300 MHz, DMSO-d₆) δ 12.08 (s, 1H, D₂O exchangeable), 8.53 (s, 2H), 8.07 (d, J=1.6 Hz, 1H), 7.00 (s, 2H), 3.97 (s, 3H), 3.88 (s, 6H), 3.70 (s, 3H), 2.85 (d, J=7.0 Hz, 2H), 1.36-1.17 (m, 1H), 0.67-0.55 (m, 2H), 0.44-0.33 (m, 2H); MS (ES+): 436.3 (M+1); Analysis calculated for C₂₂H₂₅N₇O₃·2.25 H₂O·1.35 HCl: C, 50.31; H, 5.92; Cl, 9.11; N, 18.67; Found: C, 50.51; H, 5.87; Cl, 8.97; N, 18.62.

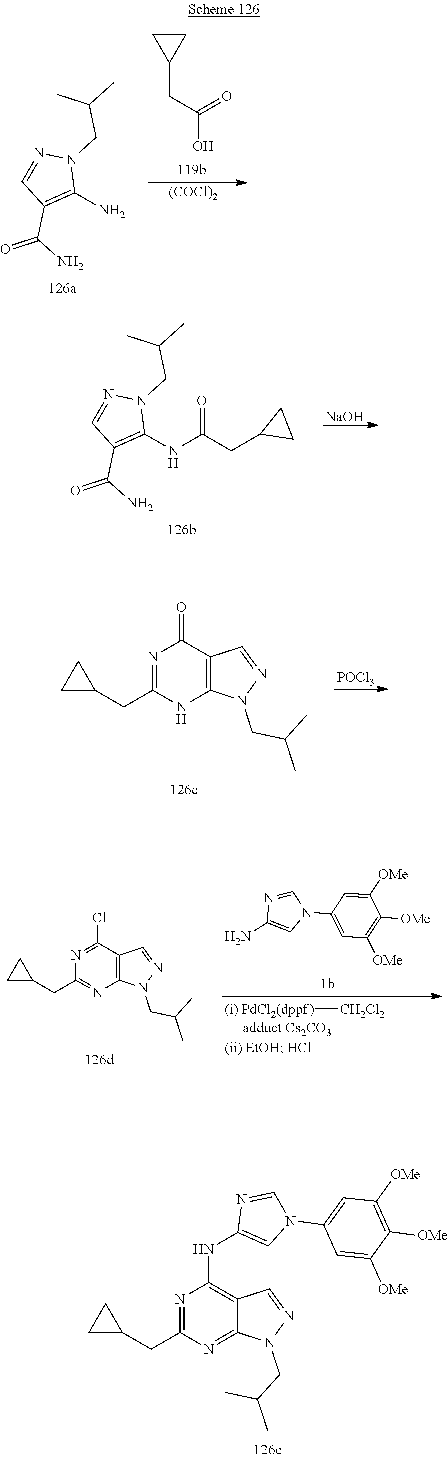

### Preparation of 6-(cyclopropylmethyl)-1-isobutyl-N-(1-(3,4,5-trimethoxyphenyl)-1H-imidazol-4-yl)-1H-pyrazolo[3,4-d]pyrimidin-4-amine (126e)

### Step-1: Preparation of 5-(2-cyclopropylacetamido)-1-isobutyl-1H-pyrazole-4-carboxamide (126b)

Compound 126b was prepared according to the procedure reported in step-1 of scheme 27, using 2-cyclopropylacetic acid (119b) (3.08 g, 30.76 mmol) in DCM (60 mL), oxalyl chloride (11.71 g, 92.28 mmol), DMF (2 drops) and stirring at RT for 1.5 h to afford after work up 2-cyclopropylacetyl chloride (3.63 g). To a solution of 5-amino-1-isobutyl-1H-pyrazole-4-carboxamide (126a) (2.8 g, 15.37 mmol; CAS #959432-42-9) in 1,4-dioxane (60 mL) was added 2-cyclopropylacetyl chloride (3.63 g) in 1,4-dioxane (36 mL) at RT and stirring at RT for 12 h to afford after work up and purification using flash column chromatography [silica gel, eluting with MeOH in DCM from 0-5%] 5-(2-cyclopropylacetamido)-1-isobutyl-1H-pyrazole-4-carboxamide (126b) (0.3 g, 7% yield) as an off-white solid and was used as such for the next step; ¹H NMR (300 MHz, DMSO-d₆) δ 9.78 (s, 11H), 7.85 (s, 1H), 7.24 (s, 1H), 7.03 (s, 1H), 3.70 (d, J=7.3 Hz, 2H), 2.23 (d, J=7.1 Hz, 2H), 2.17-2.02 (m, 1H), 1.04 (d, J=11.8 Hz, 1H), 0.80 (d, J=6.6 Hz, 6H), 0.53-0.45 (m, 2H), 0.27-0.19 (m, 2H).

### Step-2: Preparation of 6-(cyclopropylmethyl)-1-isobutyl-1,7-dihydro-4H-pyrazolo[3,4-d]pyrimidin-4-one (126c)

Compound 126c was prepared according to the procedure reported in step-1 of scheme 7, from 5-(2-cyclopropylacetamido)-1-isobutyl-1H-pyrazole-4-carboxamide (126b) (1.3 g, 4.92 mmol) in aqueous NaOH (2N, 12.25 mL) and heating at 70° C. for 0.5 h to afford after work up 6-(cyclopropylmethyl)-1-isobutyl-1,7-dihydro-4H-pyrazolo[3,4-d]pyrimidin-4-one (126c) (1.15 g, 95% yield) as an off-white solid; ¹H NMR (300 MHz, DMSO-d₆) δ 12.06-12.00 (m, 1H), 8.01 (s, 1H), 4.07 (d, J=7.7 Hz, 2H), 2.56-2.44 (m, 2H), 2.25-2.17 (m, 1H), 1.16 (s, 1H), 0.84 (d, J=7.1 Hz, 6H), 0.52-0.43 (m, 2H), 0.31-0.23 (m, 2H).

### Step-3: Preparation of 4-chloro-6-(cyclopropylmethyl)-1-isobutyl-1H-pyrazolo[3,4-d]pyrimidine (126d)

Compound 126d was prepared according to the procedure reported in step-3 of scheme 7, from 6-(cyclopropylmethyl)-1-isobutyl-1,7-dihydro-4H-pyrazolo[3,4-d]pyrimidin-4-one (126c) (1.15 g, 4.67 mmol) using POCl₃. (40.81 g, 266.12 mmol) and heating to 100° C. for 1 h to afford after work up 4-chloro-6-(cyclopropylmethyl)-1-isobutyl-1H-pyrazolo[3,4-d]pyrimidine (126d) (1.15 g, 93% yield) as an oily mass, and was used as such in the next step.

### Step-4: Preparation of 6-(cyclopropylmethyl)-1-isobutyl-N-(1-(3,4,5-trimethoxyphenyl)-1H-imidazol-4-yl)-1H-pyrazolo[3,4-d]pyrimidin-4-amine (126e)

Compound 126e was prepared according to the procedure reported in step-4 of scheme 112, from 4-chloro-6-(cyclopropylmethyl)-1-isobutyl-1H-pyrazolo[3,4-d]pyrimidine (126d) (1.15 g crude, 4.34 mmol) in 1,4-dioxane (23 mL) using 1-(3,4,5-trimethoxyphenyl)-1H-imidazol-4-amine (1b) 1.19 g, 4.77 mmol), cesium carbonate (2.83 g, 8.68 mmol), PdCl₂(dppf)-CH₂Cl₂ adduct (0.177 g, 0.21 mmol) and heating at 100° C. for 4 h to afford after work up and purification using flash column chromatography [silica gel, eluting with MeOH in DCM from 0-10%] 6-(cyclopropylmethyl)-1-isobutyl-N-(1-(3,4,5-trimethoxyphenyl)-1H-imidazol-4-yl)-1H-pyrazolo[3,4-d]pyrimidin-4-amine (126e) (0.450 g, 22% yield) free base as an off-white solid; ¹H NMR (300 MHz, DMSO-d₆) δ 10.84 (s, 1H), 8.40 (s, 1H), 8.17 (d, J=12.4 Hz, 2H), 6.92 (s, 2H), 4.11 (d, J=7.0 Hz, 2H), 3.87 (s, 6H), 3.69 (s, 3H), 2.75 (d, J=7.0 Hz, 2H), 2.29-2.19 (m, 1H), 1.30-1.24 (m, 1H), 0.84 (d, J=6.6 Hz, 6H), 0.55-0.46 (m, 2H), 0.35-0.27 (m, 2H). The free base of compound 126e was converted to its HCl salt by dissolving (0.450 g, 0.94 mmol) in EtOH (9 mL), adding 14% HCl in EtOH (0.9 mL) and stirring for 1 h at RT to afford after work up and purification using reverse phase column chromatography [C₁₈ (50 g), eluting with ACN in water (containing 0.1% HCl) from 0-55%] 6-(cyclopropylmethyl)-1-isobutyl-N-(1-(3,4,5-trimethoxyphenyl)-1H-imidazol-4-yl)-1H-pyrazolo[3,4-d]pyrimidin-4-amine (126e) (200 mg, 42% yield) HCl salt as a off-white solid; ¹H NMR (300 MHz, DMSO-d₆) δ 11.07 (s, 1H, D₂O exchangeable), 8.41 (s, 1H), 8.27 (s, 1H), 8.12 (s, 1H), 6.94 (s, 2H), 4.13 (d, J=7.2 Hz, 2H), 3.87 (s, 6H), 3.70 (s, 3H), 2.77 (d, J=6.9 Hz, 2H), 2.32-2.15 (m, 1H), 1.36-1.16 (m, 1H), 0.85 (d, J=6.7 Hz, 6H), 0.60-0.44 (m, 2H), 0.39-0.25 (m, 2H); MS (ES+): 478.3 (M+1); (ES−): 476.2 (M−1).

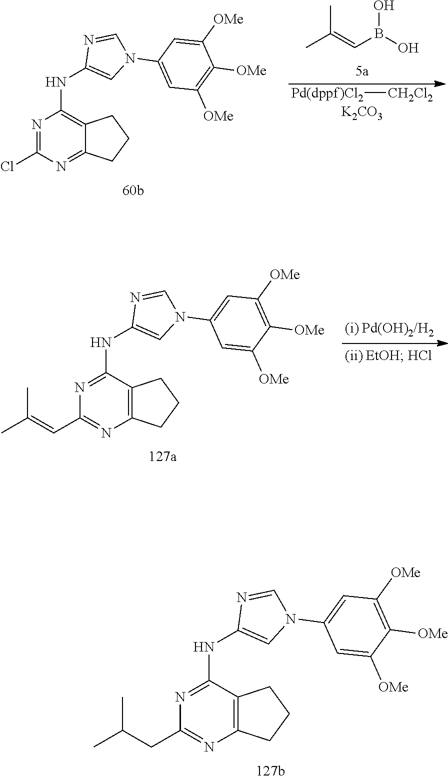

### Preparation of 2-isobutyl-N-(1-(3,4,5-trimethoxyphenyl)-1H-imidazol-4-yl)-6,7-dihydro-5H-cyclopenta[d]pyrimidin-4-amine (127b)

### Step-1: Preparation of 2-(2-methylprop-1-en-1-yl)-N-(1-(3,4,5-trimethoxyphenyl)-1H-imidazol-4-yl)-6,7-dihydro-5H-cyclopenta[d]pyrimidin-4-amine (127a)

Compound 127a was prepared according to the procedure reported in step-2 of scheme 3, from 2-chloro-N-(1-(3,4,5-trimethoxyphenyl)-1H-imidazol-4-yl)-6,7-dihydro-5H-cyclopenta[d]pyrimidin-4-amine (60b) (2.8 g, 6.97 mmol) in 1,4-dioxane (56 mL) using (2-methylprop-1-en-1-yl)boronic acid (5a) (0.87 g, 8.7 mmol), a solution of potassium carbonate (2.88 g, 20.90 mmol) in water (2.8 mL), Pd(dppf)Cl₂—CH₂Cl₂ adduct (1.13 g, 1.39 mmol) and stirring at 100° C. for 12 h under nitrogen. This gave after work up and purification using flash column chromatography [silica gel, eluting with methanol in DCM from 0-5%] followed by crystallization using MeOH (50 mL) 2-(2-methylprop-1-en-1-yl)-N-(1-(3,4,5-trimethoxyphenyl)-1H-imidazol-4-yl)-6,7-dihydro-5H-cyclopenta[d]pyrimidin-4-amine (127a) (1.5 g, 51% yield) as an off-white solid; ¹H NMR (400 MHz, DMSO-d₆) δ 9.20 (s, 1H), 8.13 (d, J=1.6 Hz, 1H), 7.88 (d, J=1.6 Hz, 1H), 6.88 (s, 2H), 6.29-6.23 (m, 1H), 3.88 (s, 6H), 3.68 (s, 3H), 2.90-2.67 (m, 4H), 2.27 (d, J=1.3 Hz, 3H), 2.06-1.94 (m, 2H), 1.90 (d, J=1.5 Hz, 3H).

### Step-2: Preparation of 2-isobutyl-N-(1-(3,4,5-trimethoxyphenyl)-1H-imidazol-4-yl)-6,7-dihydro-5H-cyclopenta[d]pyrimidin-4-amine (127b)

Compound 127b was prepared according to the procedure reported in step-3 of scheme 1, from 2-(2-methylprop-1-en-1-yl)-N-(1-(3,4,5-trimethoxyphenyl)-1H-imidazol-4-yl)-6,7-dihydro-5H-cyclopenta[d]pyrimidin-4-amine (127a) (1.5 g, 3.56 mmol) in MeOH:DCM (9:1) using 20% Pd(OH)₂ on carbon (50% wet) (1.01 g, 0.71 mmol), acetic acid (0.5 mL) and stirring under hydrogen gas (60 psi) at RT for 12 h. This gave after work up and trituration with MeOH (20 mL) 2-isobutyl-N-(1-(3,4,5-trimethoxyphenyl)-1H-imidazol-4-yl)-6,7-dihydro-5H-cyclopenta[d]pyrimidin-4-amine (127b) (0.8 g, 53% yield) free base as a grey solid ¹H NMR (300 MHz, DMSO-d₆) δ 10.33 (s, 1H), 8.25 (s, 1H), 8.01 (s, 1H), 6.91 (s, 2H), 3.88 (s, 6H), 3.69 (s, 3H), 2.96-2.82 (m, 4H), 2.74 (d, J=7.1 Hz, 2H), 2.38-2.23 (m, 1H), 2.14-2.03 (m, 2H), 0.97 (d, J=6.6 Hz, 6H). The free base of compound 127b was converted to its HCl salt by dissolving (1.0 g, 2.36 mmol) in EtOH (10 mL), adding 14% HCl in EtOH (2 mL) and stirring for 1 h at RT to afford after work up 2-isobutyl-N-(1-(3,4,5-trimethoxyphenyl)-1H-imidazol-4-yl)-6,7-dihydro-5H-cyclopenta[d]pyrimidin-4-amine (127b) (0.820 g, 75% yield) HCl salt as a grey solid; ¹H NMR (300 MHz, DMSO-d₆) δ 14.97 (s, 1H, D₂O exchangeable), 11.26 (s, 1H, D₂O exchangeable), 8.34 (d, J=1.5 Hz, 1H), 8.04 (d, J=1.6 Hz, 1H), 6.93 (s, 2H), 3.88 (s, 6H), 3.70 (s, 3H), 3.06 (t, 2H), 2.94 (t, 2H), 2.86 (d, J=7.0 Hz, 2H), 2.39-2.24 (m, 1H), 2.24-2.06 (m, 2H), 0.99 (d, J=6.7 Hz, 6H); MS (ES+): 424.3 (M+1); (ES−): 422.1 (M−1); Analysis calculated for C₂₃H₂₉N₅O₃·2HCl·H₂O: C, 53.70; H, 6.47; Cl, 13.78; N, 13.61; Found: C, 53.68; H, 6.45; Cl, 13.51; N, 13.49.

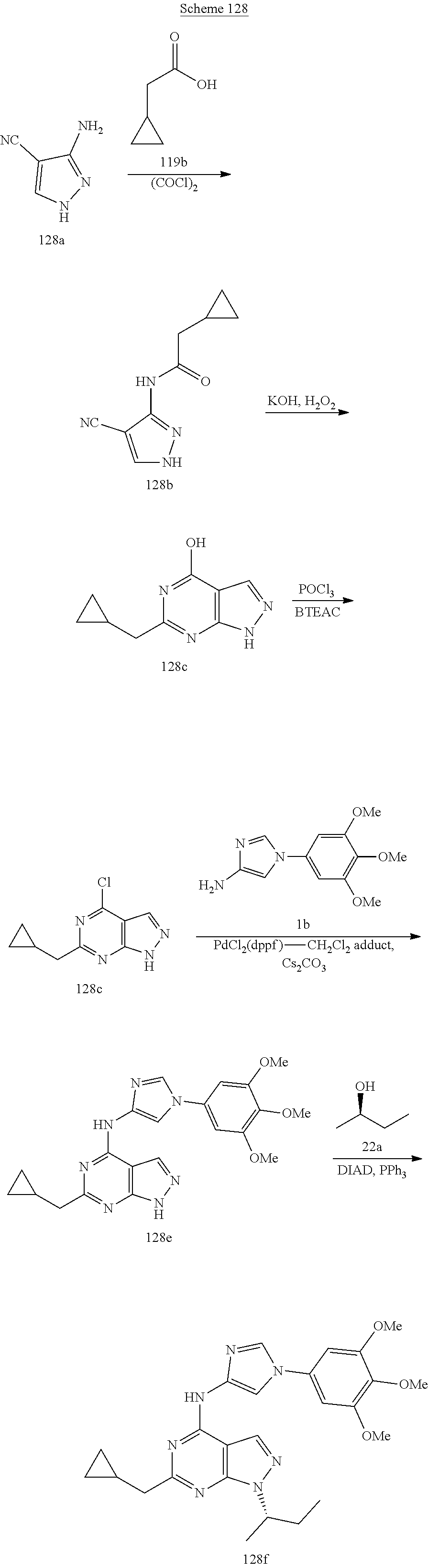

### Preparation of(S)-1-(sec-butyl)-6-(cyclopropylmethyl)-N-(1-(3,4,5-trimethoxyphenyl)-1H-imidazol-4-yl)-1H-pyrazolo[3,4-d]pyrimidin-4-amine (128f)

### Step-1: Preparation of N-(4-cyano-1H-pyrazol-3-yl)-2-cyclopropylacetamide (128b)

Compound 128b was prepared according to the procedure reported in step-1 of scheme 27, using 2-cyclopropylacetic acid (119b) (2.8 g, 27.97 mmol) in DCM (56 mL), oxalyl chloride (10.65 g, 83.91 mmol), DMF (4-5 drops) and stirring at RT for 4.5 h to afford after work up 2-cyclopropylacetyl chloride (3.35 g). To a solution of 3-amino-1H-pyrazole-4-carbonitrile (128a) (2.0 g, 18.50 mmol) in 1,4-dioxane (100 mL) was added 2-cyclopropylacetyl chloride (3.3 g, 27.83 mmol) in 1,4-dioxane (33 mL) at RT and stirring at 60° C. for 14 h to afford after work up N-(4-cyano-1H-pyrazol-3-yl)-2-cyclopropylacetamide (128b) (3.0 g 85% yield), which was used as such for the next step; MS (ES+): 191.3 (M+1); (ES−): 189.1 (M−1)

### Step-2: Preparation of 6-(cyclopropylmethyl)-1H-pyrazolo[3,4-d]pyrimidin-4-ol (128c)

Compound 128c was prepared according to the procedure reported in step-3 of scheme 121, from N-(4-cyano-1H-pyrazol-3-yl)-2-cyclopropylacetamide (128b) (3.0 g, 15.77 mmol) in aqueous KOH (5N, 37.87 mL, 189.37 mmol) using H₂O₂ (30% in water, 60 mL) and stirring at 75° C. for 2 h to afford after work up and purification 6-(cyclopropylmethyl)-1H-pyrazolo[3,4-d]pyrimidin-4-ol (128c) (1.4 g, 47% yield) as an off-white solid; ¹H NMR (300 MHz, DMSO-d₆) δ 13.61 (s, 1H), 11.94 (s, 1H), 7.99 (s, 1H), 2.48 (s, 2H), 1.27-1.07 (m, 1H), 0.54-0.40 (m, 2H), 0.31-0.20 (m, 2H).

### Step-3: Preparation of 4-chloro-6-(cyclopropylmethyl)-1H-pyrazolo[3,4-d]pyrimidine (128d)

To a stirred solution of 6-(cyclopropylmethyl)-1H-pyrazolo[3,4-d]pyrimidin-4-ol (128c) (0.5 g, 2.63 mmol) in acetonitrile (8.0 mL) was added benzyltriethylammonium chloride (1.19 g, 5.25 mmol). The mixture was heated to 50° C., added N,N-dimethylaniline (0.47 g, 3.94 mmol), followed by POCl₃ (4.03 g, 26.28 mmol) dropwise at 50° C.-65° C., and stirred at 75° C. for 0.5 h. The reaction mixture was cooled to RT, poured in ice water and the pH was adjusted to neutral using saturated aqueous NaHCO₃ solution and was extracted with DCM (2×100 mL). Combined organics were washed with brine, dried, filtered and concentrated in vacuo. The residue obtained was purified using flash column chromatography [silica gel, eluting with MeOH in DCM from 0-2.5%] to give 4-chloro-6-(cyclopropylmethyl)-1H-pyrazolo[3,4-d]pyrimidine (128d) (0.38 g, 69% yield) as an oily mass; ¹H NMR (300 MHz, DMSO-d₆) δ 14.31 (s, 1H), 8.36 (d, J=1.3 Hz, 1H), 2.84 (d, J=7.0 Hz, 2H), 1.26-1.20 (m, 1H), 0.57-0.45 (m, 2H), 0.29-0.14 (m, 2H).

### Step-4: Preparation of 6-(cyclopropylmethyl)-N-(1-(3,4,5-trimethoxyphenyl)-1H-imidazol-4-yl)-1H-pyrazolo[3,4-d]pyrimidin-4-amine (128e)

Compound 128e was prepared according to the procedure reported in step-4 of scheme 7, from 4-chloro-6-(cyclopropylmethyl)-1H-pyrazolo[3,4-d]pyrimidine (128d) (2.65 g, 12.70 mmol) in 1,4-dioxane (50 mL) using 1-(3,4,5-trimethoxyphenyl)-1H-imidazol-4-amine (1b) (2.98 g, 11.98 mmol), cesium carbonate (7.8 g, 23.96 mmol), PdCl₂(dppf)-CH₂Cl₂ adduct (0.48 g, 0.59 mmol) and heating at 95° C. for 12 h to afford after work up and purification using flash column chromatography [silica gel, eluting with MeOH in DCM from 0-5%] 6-(cyclopropylmethyl)-N-(1-(3,4,5-trimethoxyphenyl)-1H-imidazol-4-yl)-1H-pyrazolo[3,4-d]pyrimidin-4-amine (128e) (1.0 g, 19% yield) as a yellow solid; ¹H NMR (300 MHz, DMSO-d₆) δ 13.36 (s, 1H), 10.77 (s, 1H), 8.39 (s, 1H), 8.16 (d, J=13.0 Hz, 2H), 6.92 (s, 2H), 3.87 (s, 6H), 3.70 (s, 3H), 2.73 (d, J=6.9 Hz, 2H), 1.20-0.76 (m, 1H), 0.58-0.46 (m, 2H), 0.34-0.25 (m, 2H).

### Step-5: (S)-1-(sec-butyl)-6-(cyclopropylmethyl)-N-(1-(3,4,5-trimethoxyphenyl)-1H-imidazol-4-yl)-1H-pyrazolo[3,4-d]pyrimidin-4-amine (128f)

Compound 128f was prepared according to the procedure reported in step-2 of scheme 15, from 6-(cyclopropylmethyl)-N-(1-(3,4,5-trimethoxyphenyl)-1H-imidazol-4-yl)-1H-pyrazolo[3,4-d]pyrimidin-4-amine (128e) (1.0 g, 2.37 mmol) in THF (50 mL) using triphenylphosphine (3.11 g, 11.86 mmol), (R)-butan-2-ol (22a) (0.52 g, 7.11 mmol), DIAD (1.43 g, 7.11 mmol) and stirring at RT for 0.5 h. This gave after work up and purification using flash column chromatography [silica gel, eluting with MeOH in DCM from 0-4%] followed by reverse phase column chromatography [C18 (50 g), eluting with ACN in water (containing 0.1% HCl) from 0-55%] (S)-1-(sec-butyl)-6-(cyclopropylmethyl)-N-(1-(3,4,5-trimethoxyphenyl)-1H-imidazol-4-yl)-1H-pyrazolo[3,4-d]pyrimidin-4-amine (128f) (0.6 g, 21% yield) HCl salt as a white solid; ¹H NMR (300 MHz, DMSO-d₆) δ 11.12 (s, 1H, D₂O exchangeable), 8.44 (s, 1H), 8.29 (s, 1H), 8.13 (s, 1H), 6.95 (s, 2H), 4.95-4.69 (m, 1H), 3.88 (s, 6H), 3.70 (s, 3H), 2.78 (d, J=6.9 Hz, 2H), 1.99-1.74 (m, 2H), 1.45 (d, J=6.7 Hz, 3H), 1.34-1.21 (m, 1H), 0.67 (t, J=7.3 Hz, 3H), 0.61-0.50 (m, 2H), 0.40-0.29 (m, 2H); MS (ES+): 478.2 (M+1).

### Example 129

Biochemical assays to measure the inhibitory effects of the compounds were performed by ThermoFisher Scientific (Life Technologies). ALK2 inhibition was tested using LanthaScreen™ Eu Kinase Binding Assay screening protocol. Values generated from the enzymatic assay are shown in table below.

TABLE 1
Measured % inhibition values at 1 micromolar concentration.

| | % Inhibition | | % Inhibition | | % Inhibition |
|---|---|---|---|---|---|
| Compound | ALK2 @ 1 uM | Compound | ALK2 @ 1 uM | Compound | ALK2 @ 1 uM |
| 3d | +++ | 19d | +++ | 44e | +++ |
| 4a | +++ | 20c | +++ | 45e | +++ |
| 5b | +++ | 21c | +++ | 27f | +++ |
| 6a | +++ | 13b | +++ | 28e | +++ |
| 2d | +++ | 31b | +++ | 29a | +++ |
| 8b | +++ | 32d | +++ | 30e | +++ |
| 1f | +++ | 33d | +++ | 57e | +++ |
| 7e | +++ | 22d | +++ | 58a | +++ |
| 9b | +++ | 39d | +++ | 59e | +++ |
| 14f | +++ | 43d | +++ | 38b | +++ |
| 15e | +++ | 42d | +++ | 46d | +++ |
| 16c | +++ | 34d | +++ | 47a | +++ |
| 11b | +++ | 23c | +++ | 40b | +++ |
| 17c | +++ | 35d | +++ | 48a | +++ |
| 18c | +++ | 24c | +++ | 41a | +++ |
| 12b | +++ | 36e | +++ | 49b | +++ |
| 26a | +++ | 104a | +++ | 50a | +++ |
| 25b | +++ | | | 51c | +++ |
| | % Inhibition | | % Inhibition | | % Inhibition |
| Compound | ALK2 @ 1 uM | Compound | ALK2 @ 1 uM | Compound | ALK2 @ 1 uM |
| 52b | +++ | 74c | +++ | 93a | +++ |
| 53c | +++ | 79a | + | 94a | +++ |
| 54a | +++ | 77a | +++ | 96a | +++ |
| 55c | +++ | 76c | + | 98a | +++ |
| 60c | +++ | 81a | +++ | 97c | +++ |
| 56a | +++ | 82c | +++ | 64c | +++ |
| 61c | +++ | 78c | +++ | 75a | +++ |
| 69a | +++ | 83a | +++ | 95c | +++ |
| 63c | +++ | 84c | +++ | 99a | +++ |
| 65c | + | 85a | +++ | 100b | +++ |
| 68b | +++ | 86a | +++ | 103c | +++ |
| 67a | +++ | 87a | +++ | 106a | +++ |
| 105a | +++ | 89a | +++ | 102e | +++ |
| 70b | +++ | 88c | +++ | 107a | +++ |
| 71c | +++ | 90b | +++ | 108a | +++ |
| 80c | +++ | 91a | +++ | 109a | + |
| 73a | +++ | 92c | +++ | 110d | + |
| 72a | +++ | 101b | +++ | 111d | +++ |
| 112d | +++ | 118f | +++ | 124e | +++ |
| 113d | +++ | 119e | +++ | 125e | +++ |
| 114e | +++ | 120f | +++ | 122e | +++ |
| 115b | +++ | 121f | +++ | 117e | +++ |
| 116d | +++ | 123e | +++ | 126e | +++ |
| | | | | 127b | +++ |

One (+) is used to denote compound with values of less than 50% inhibition; Three (+++) indicate compounds with an IC₅₀ value greater than 50% inhibition.

### INCORPORATION BY REFERENCE

All of the U.S. patents, and U.S. and PCT published patent applications cited herein are hereby incorporated by reference, except for any claims, definitions, subject matter disclaimers or disavowals, and except to the extent that the incorporated material is inconsistent with the express disclosure herein, in which case the language in this disclosure controls.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A compound represented by formula (I) or formula (II): 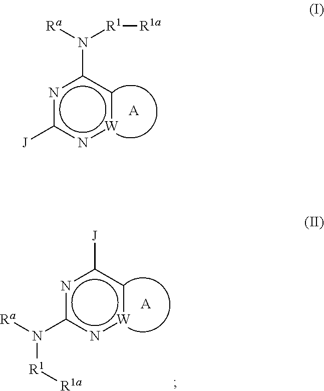
or a pharmaceutically acceptable salt thereof;
wherein:
A is a fused optionally substituted aromatic ring, heteroaromatic ring, cycloalkyl ring, cycloalkenyl ring, heterocycloalkyl ring, or heterocycloalkenyl ring;
W is C or N;
R^{a} represents H or alkyl;
R¹ represents heteroarylene;
R^{1a} represents H or optionally substituted —C(O)alkyl, —C(O)aryl, —C(O)heteroaryl, —C(O)O(alkyl), —C(O)(heterocyclyl), —C(O)NR^{x}R^{y}, alkyl, cycloalkyl, cycloalkenyl, heterocycloalkyl, aryl, or heteroaryl;
J represents optionally substituted alkyl, alkenyl, haloalkyl, hydroxyalkyl, alkoxyalkyl, aminoalkyl, cycloalkyl, (cycloalkyl)alkyl, heterocycloalkyl, (heterocycloalkyl)alkyl, or heterocycloalkenyl;
further wherein when J is heterocycloalkyl or heterocycloalkenyl, the point of attachment in J to the rest of the compound is a carbon atom; and
R^{x} and R^{y} each independently represent H alkyl, aralkyl, heteroaralkyl, aryl, heteroaryl, cycloalkyl, heterocycloalkyl, (cycloalkyl)alkyl, (heterocycloalkyl)alkyl, or hydroxyalkyl.

2. The compound of claim 1, wherein the compound is represented by formula (Ia) or formula (IIa): 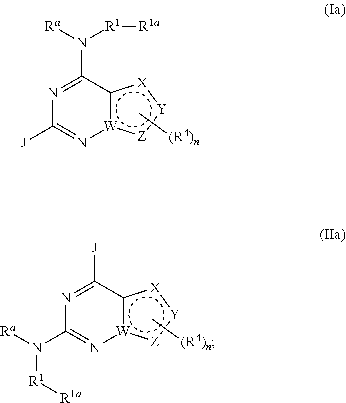
wherein:
W is C or N;
valence permitting, each of X, Y, and Z independently represent CH, CH₂, CO, N, NH, O, S, or SO₂, wherein any hydrogen of a CH, CH₂, or NH group is optionally replaced by an occurrence of R⁴;
R⁴, independently for each occurrence, represents halo, cyano, —CH₂C(O)NH₂, —C(O)R⁵, —C(O)OR⁵, —S(O)₂R⁵, or optionally substituted alkyl, alkenyl, haloalkyl, hydroxyalkyl, aminoalkyl, alkoxy, aryl, aralkyl, heteroaryl, heteroaralkyl, heterocycloalkyl, heterocycloalkenyl, (heterocycloalkyl)alkyl, cycloalkyl, (cycloalkyl)alkyl, halocycloalkyl, hydroxycycloalkyl, aminocycloalkyl, aryloxy, heteroaryloxy, arylalkyloxy, or heteroarylalkyloxy;
R⁵, independently for each occurrence, represents H or optionally substituted alkyl, aralkyl, aryl, heteroaralkyl, heteroaryl, cycloalkyl, heterocycloalkyl, (cycloalkyl)alkyl, or (heterocycloalkyl)alkyl; and
n is an integer from 0-4, as permitted by valence.

3. The compound of claim 2, wherein the compound is represented by formula (Ib) or (IIb): 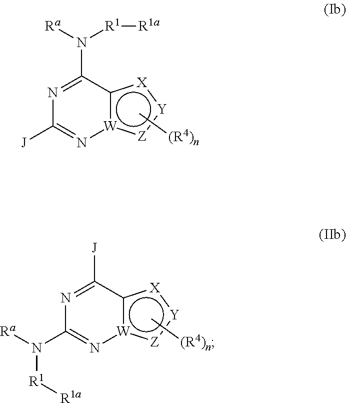
wherein each of X, Y, and Z independently represent CH, N, NH, O, S, or SO₂.

4. The compound of claim 3, wherein the compound is represented by formula (Ic) or (IIc): 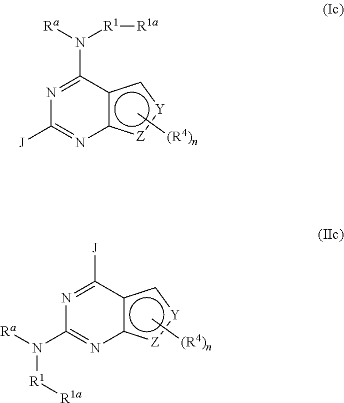
wherein each of Y and Z are independently selected from the group consisting of O, N, NH, and S.

5. The compound of claim 4, wherein Y is N; and Z is NH.

6. The compound of claim 3, wherein the compound is represented by formula (Id) or (IId): 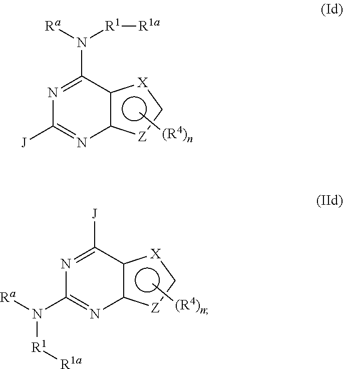
wherein at least one of X and Z is selected from the group consisting of O, N, NH, and S.

7. The compound of claim 6, wherein one of X and Z is selected from the group consisting of O, NH, and S; and the other of X and Z is CH.

8. The compound of claim 6 or 7, wherein X is selected from the group consisting of O, NH, and S.

9. The compound of claim 6 or 7, wherein Z is selected from the group consisting of O, NH, and S.

10. The compound of claim 6, wherein one of X and Z is NH; and the other of X and Z is CH.

11. The compound of claim 6, wherein one of X and Z is O; and the other of X and Z is CH.

12. The compound of claim 6, wherein one of X and Z is S; and the other of X and Z is CH.

13. The compound of claim 6, wherein each of X and Z are selected from the group consisting of O, N, NH, and S.

14. The compound of claim 13, wherein one of X and Z is N; and the other of X and Z is NH.

15. The compound of claim 13, wherein one of X and Z is S; and the other of X and Z is N.

16. The compound of claim 2, wherein the compound is represented by formula (Ie) or (IIe): 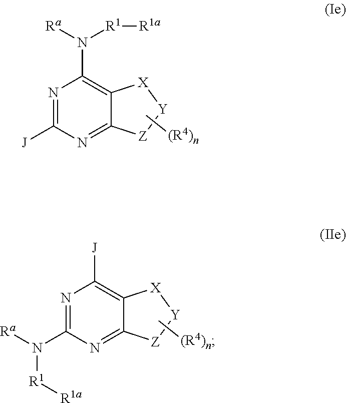
wherein X, Y, and Z independently represent CH₂, CO, NH, O, S, or SO₂.

17. The compound of claim 16, wherein each of X, Y, and Z is CH₂.

18. The compound of claim 16, wherein one of X, Y, and Z is O.

19. The compound of any one of claims 2-18, wherein n is 0 or 1.

20. The compound of claim 1, wherein the compound is represented by formula (If) or formula (IIf): 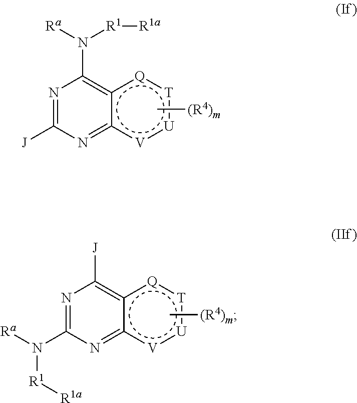
wherein:
valence permitting, each of Q, T, U, and V independently represent CH, CH₂, N, NH, O, or SO₂, wherein any hydrogen of a CH, CH₂, or NH group is optionally replaced by an occurrence of R⁴;
R⁴, independently for each occurrence, represents halo, cyano, —CH₂C(O)NH₂, —C(O)R⁵, —C(O)OR⁵, —S(O)₂R⁵, or optionally substituted alkyl, alkenyl, haloalkyl, hydroxyalkyl, aminoalkyl, alkoxy, aryl, aralkyl, heteroaryl, heteroaralkyl, heterocycloalkyl, heterocycloalkenyl, (heterocycloalkyl)alkyl, cycloalkyl, (cycloalkyl)alkyl, halocycloalkyl, hydroxycycloalkyl, aminocycloalkyl, aryloxy, heteroaryloxy, arylalkyloxy, or heteroarylalkyloxy;
R⁵, independently for each occurrence, represents H or optionally substituted alkyl, aralkyl, aryl, heteroaralkyl, heteroaryl, cycloalkyl, heterocycloalkyl, (cycloalkyl)alkyl, or (heterocycloalkyl)alkyl; and
m is an integer from 0-4, as permitted by valence.

21. The compound of claim 20, wherein the compound is represented by formula (Ig) or (IIg): 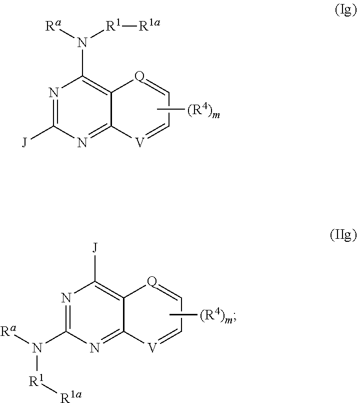
wherein Q represents CH or N; and V represents CH or N.

22. The compound of claim 21, wherein Q is N; and V is CH.

23. The compound of claim 21, wherein Q is CH; and V is N.

24. The compound of claim 21, wherein the compound is represented by formula (Ih) or (IIh):

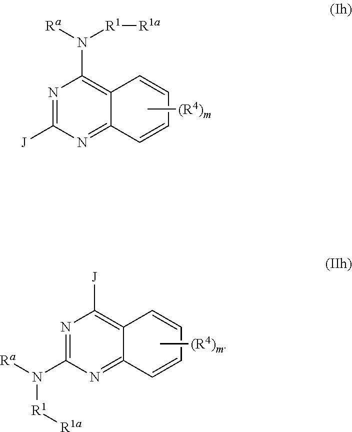

25. The compound of claim 20, wherein the compound is represented by formula (Ij) or (IIj): 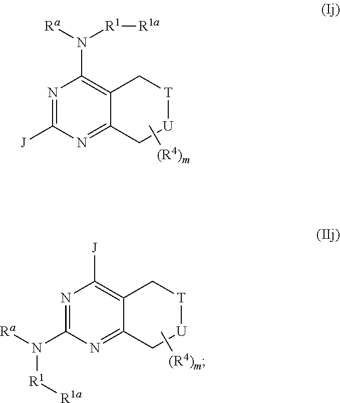
wherein T represents CH₂, NH, O, or SO₂; and U represents CH₂, NH, O, or SO₂.

26. The compound of claim 25, wherein the compound is represented by formula (Ik) or (IIk):

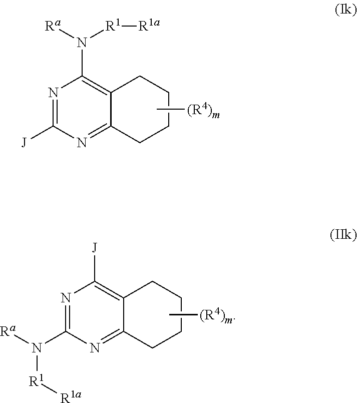

27. The compound of claim 25, wherein T is NH; and U is CH₂.

28. The compound of claim 25, wherein T is CH₂; and U is NH.

29. The compound of any one of claims 20-28, wherein m is 0 or 1.

30. The compound of any one of claims 1-29, wherein R⁴, if present, is halo, —C(O)O(alkyl), or is selected from the group consisting of optionally substituted alkyl, alkoxy, aryl, heterocycloalkyl, cycloalkyl, and (cycloalkyl)alkyl.

31. The compound of any one of claims 1-30, wherein R⁴, if present, is optionally substituted alkyl, cycloalkyl, or alkoxy.

32. The compound of any one of claims 1-31, wherein R^{a} is H.

33. The compound of any one of claims 1-32, wherein R¹ is a nitrogen-containing heteroarylene.

34. The compound of any one of claims 1-33, wherein R¹ is a 5-membered nitrogen-containing heteroarylene.

35. The compound of any one of claims 1-34, wherein R¹ is imidazolene.

36. The compound of any one of claims 1-35, wherein —R¹—R^{1a} represents

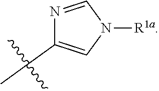

37. The compound of any one of claims 1-36, wherein R^{1a} is optionally substituted phenyl.

38. The compound of any one of claims 1-36, wherein R^{1a} is phenyl, substituted by one or more occurrences of alkoxy.

39. The compound of claim 38, wherein R^{1a} is 3,4,5-trimethoxyphenyl.

40. The compound of any one of claims 1-39, wherein J represents optionally substituted alkyl, alkenyl, cycloalkyl, or (cycloalkyl)alkyl.

41. The compound of any one of claims 1-40, wherein J represents optionally substituted branched alkyl or alkenyl.

42. The compound of any one of claims 1-41, wherein J represents isopropyl or isopropenyl.

43. The compound of any one of claims 1-40, wherein J represents optionally substituted cycloalkyl or (cycloalkyl)alkyl.

44. The compound of any one of claims 1-40, wherein J represents optionally substituted cycloalkyl.

45. The compound of any one of claims 1-39, wherein J represents optionally substituted heterocycloalkyl.

46. The compound of any one of claims 1-29, wherein:
R^{1a} is phenyl, substituted by two or more occurrences of alkoxy;
J is optionally substituted alkyl, alkenyl, cycloalkyl, or (cycloalkyl)alkyl, and
R⁴, if present, is halo, —C(O)O(alkyl), or is selected from the group consisting of optionally substituted alkyl, alkoxy, aryl, heterocycloalkyl, cycloalkyl, and (cycloalkyl)alkyl.

47. The compound of any one of claims 1-29, wherein:
R^{1a} is phenyl, substituted by two or more occurrences of alkoxy (including 3,4,5-trimethoxyphenyl);
J is optionally substituted alkyl, alkenyl, cycloalkyl, or (cycloalkyl)alkyl; and
R⁴, if present, is selected from the group consisting of optionally substituted alkyl, cycloalkyl, or alkoxy.

48. The compound of any one of claims 1-29, wherein:
R^{1a} is 3,4,5-trimethoxyphenyl;
J is optionally substituted alkyl, alkenyl, cycloalkyl, or (cycloalkyl)alkyl; and
R⁴, if present, is halo, —C(O)O(alkyl), or is selected from the group consisting of optionally substituted alkyl, alkoxy, aryl, heterocycloalkyl, cycloalkyl, and (cycloalkyl)alkyl.

49. The compound of any one of claims 1-29, wherein:
R^{1a} is 3,4,5-trimethoxyphenyl;
J is optionally substituted alkyl, alkenyl, cycloalkyl, or (cycloalkyl)alkyl; and
R⁴, if present, is selected from the group consisting of optionally substituted alkyl, cycloalkyl, or alkoxy.

50. The compound of claim 1, or a pharmaceutically acceptable salt thereof, selected from the following table:

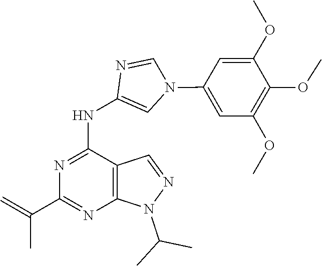 [SMILES: C=C(C)c1nc(Nc2cn(-c3cc(OC)c(OC)c(OC)c3)cn2)c2cnn(C(C)C)c2n1]

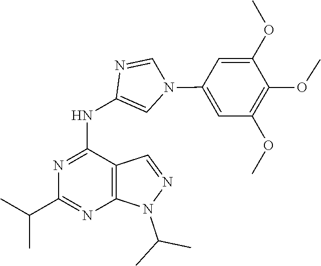 [SMILES: COc1cc(-n2cnc(Nc3nc(C(C)C)nc4c3cnn4C(C)C)c2)cc(OC)c1OC]

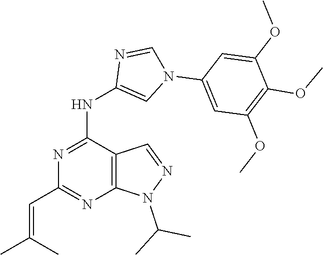 [SMILES: COc1cc(-n2cnc(Nc3nc(C=C(C)C)nc4c3cnn4C(C)C)c2)cc(OC)c1OC]

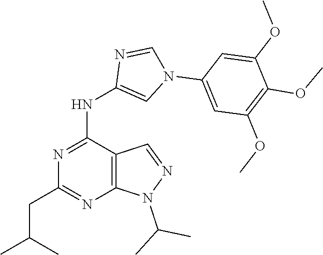 [SMILES: COc1cc(-n2cnc(Nc3nc(CC(C)C)nc4c3cnn4C(C)C)c2)cc(OC)c1OC]

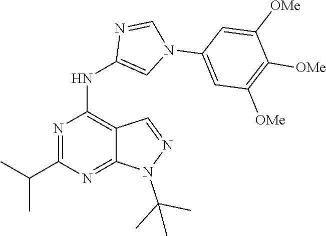 [SMILES: COc1cc(-n2cnc(Nc3nc(C(C)C)nc4c3cnn4C(C)(C)C)c2)cc(OC)c1OC]

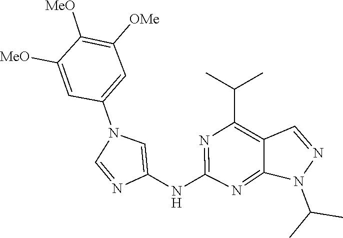 [SMILES: COc1cc(-n2cnc(Nc3nc(C(C)C)c4cnn(C(C)C)c4n3)c2)cc(OC)c1OC]

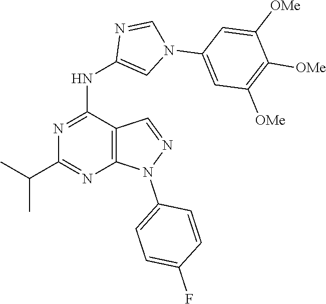 [SMILES: COc1cc(-n2cnc(Nc3nc(C(C)C)nc4c3cnn4-c3ccc(F)cc3)c2)cc(OC)c1OC]

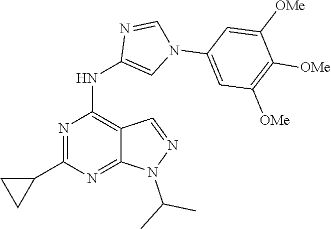 [SMILES: COc1cc(-n2cnc(Nc3nc(C4CC4)nc4c3cnn4C(C)C)c2)cc(OC)c1OC]

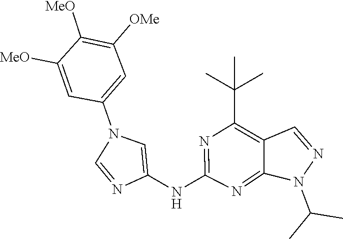 [SMILES: COc1cc(-n2cnc(Nc3nc(C(C)(C)C)c4cnn(C(C)C)c4n3)c2)cc(OC)c1OC]

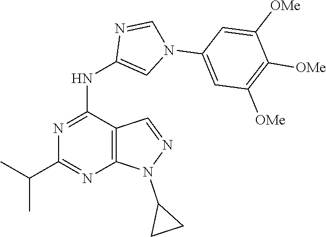 [SMILES: COc1cc(-n2cnc(Nc3nc(C(C)C)nc4c3cnn4C3CC3)c2)cc(OC)c1OC]

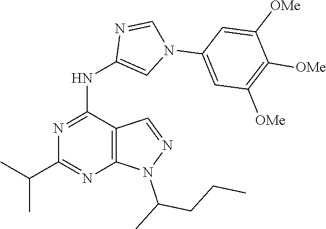 [SMILES: CCCC(C)n1ncc2c(Nc3cn(-c4cc(OC)c(OC)c(OC)c4)cn3)nc(C(C)C)nc21]

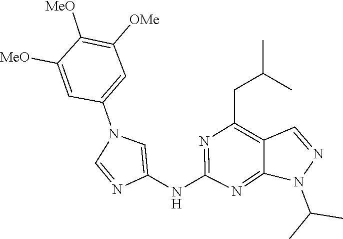 [SMILES: COc1cc(-n2cnc(Nc3nc(CC(C)C)c4cnn(C(C)C)c4n3)c2)cc(OC)c1OC]

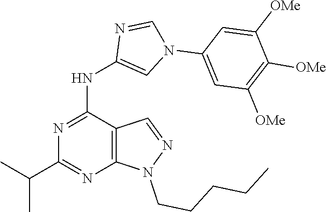 [SMILES: CCCCCn1ncc2c(Nc3cn(-c4cc(OC)c(OC)c(OC)c4)cn3)nc(C(C)C)nc21]

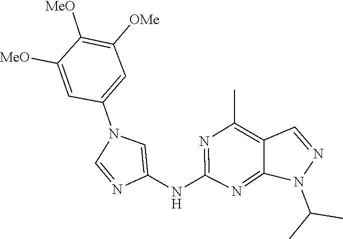 [SMILES: COc1cc(-n2cnc(Nc3nc(C)c4cnn(C(C)C)c4n3)c2)cc(OC)c1OC]

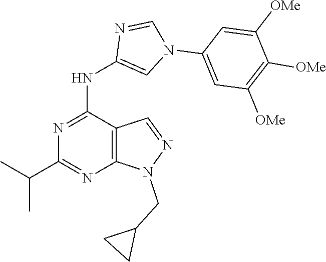 [SMILES: COc1cc(-n2cnc(Nc3nc(C(C)C)nc4c3cnn4CC3CC3)c2)cc(OC)c1OC]

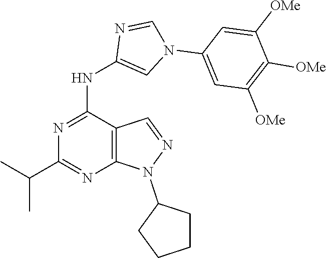 [SMILES: COc1cc(-n2cnc(Nc3nc(C(C)C)nc4c3cnn4C3CCCC3)c2)cc(OC)c1OC]

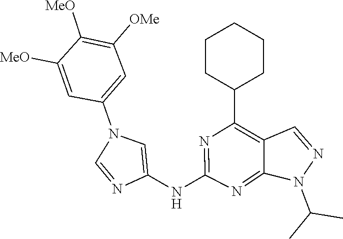 [SMILES: COc1cc(-n2cnc(Nc3nc(C4CCCCC4)c4cnn(C(C)C)c4n3)c2)cc(OC)c1OC]

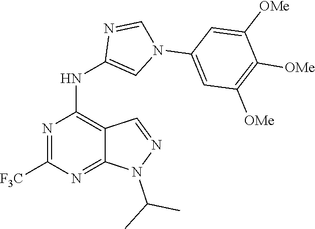 [SMILES: COc1cc(-n2cnc(Nc3nc(C(F)(F)F)nc4c3cnn4C(C)C)c2)cc(OC)c1OC]

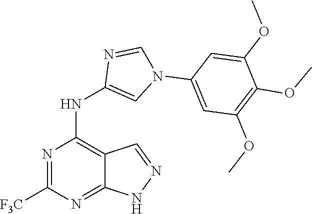 [SMILES: COc1cc(-n2cnc(Nc3nc(C(F)(F)F)nc4[nH]ncc34)c2)cc(OC)c1OC]

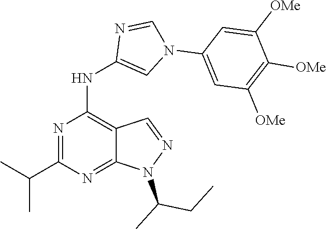 [SMILES: CC[C@@H](C)n1ncc2c(Nc3cn(-c4cc(OC)c(OC)c(OC)c4)cn3)nc(C(C)C)nc21]

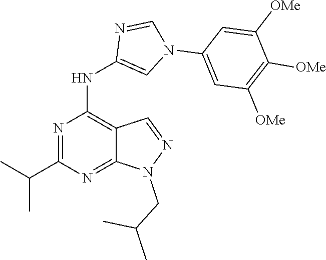 [SMILES: COc1cc(-n2cnc(Nc3nc(C(C)C)nc4c3cnn4CC(C)C)c2)cc(OC)c1OC]

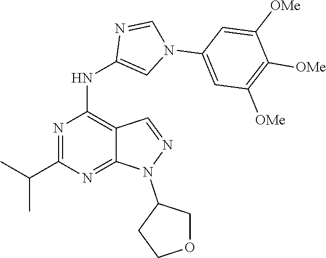 [SMILES: COc1cc(-n2cnc(Nc3nc(C(C)C)nc4c3cnn4C3CCOC3)c2)cc(OC)c1OC]

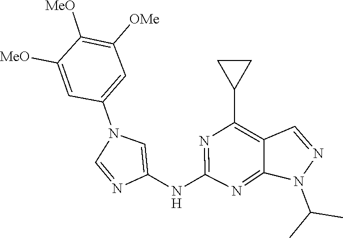 [SMILES: COc1cc(-n2cnc(Nc3nc(C4CC4)c4cnn(C(C)C)c4n3)c2)cc(OC)c1OC]

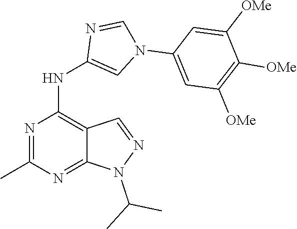 [SMILES: COc1cc(-n2cnc(Nc3nc(C)nc4c3cnn4C(C)C)c2)cc(OC)c1OC]

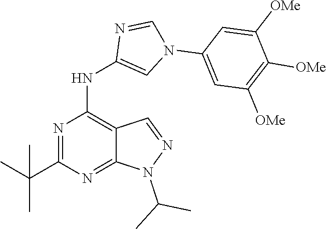 [SMILES: COc1cc(-n2cnc(Nc3nc(C(C)(C)C)nc4c3cnn4C(C)C)c2)cc(OC)c1OC]

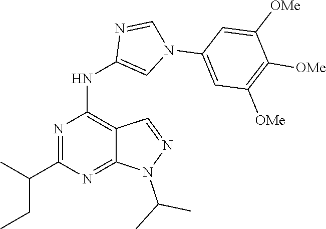 [SMILES: CCC(C)c1nc(Nc2cn(-c3cc(OC)c(OC)c(OC)c3)cn2)c2cnn(C(C)C)c2n1]

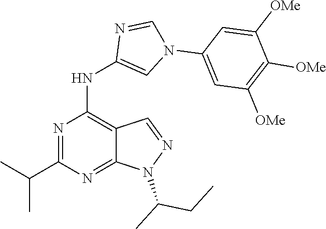 [SMILES: CC[C@H](C)n1ncc2c(Nc3cn(-c4cc(OC)c(OC)c(OC)c4)cn3)nc(C(C)C)nc21]

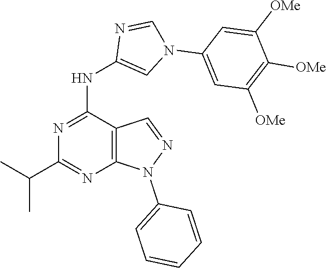 [SMILES: COc1cc(-n2cnc(Nc3nc(C(C)C)nc4c3cnn4-c3ccccc3)c2)cc(OC)c1OC]

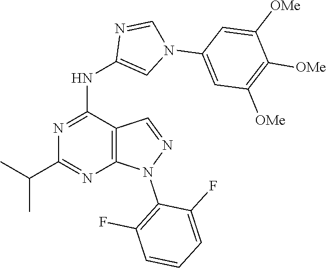 [SMILES: COc1cc(-n2cnc(Nc3nc(C(C)C)nc4c3cnn4-c3c(F)cccc3F)c2)cc(OC)c1OC]

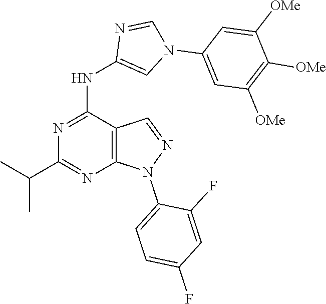 [SMILES: COc1cc(-n2cnc(Nc3nc(C(C)C)nc4c3cnn4-c3ccc(F)cc3F)c2)cc(OC)c1OC]

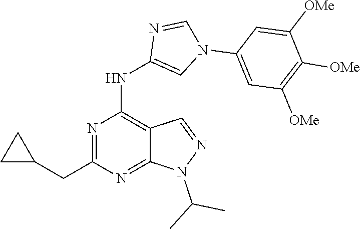 [SMILES: COc1cc(-n2cnc(Nc3nc(CC4CC4)nc4c3cnn4C(C)C)c2)cc(OC)c1OC]

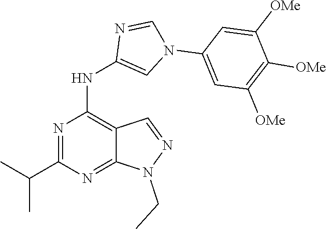 [SMILES: CCn1ncc2c(Nc3cn(-c4cc(OC)c(OC)c(OC)c4)cn3)nc(C(C)C)nc21]

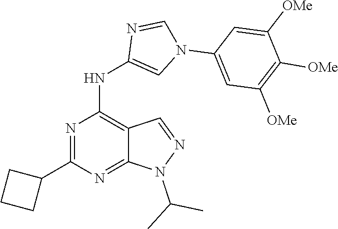 [SMILES: COc1cc(-n2cnc(Nc3nc(C4CCC4)nc4c3cnn4C(C)C)c2)cc(OC)c1OC]

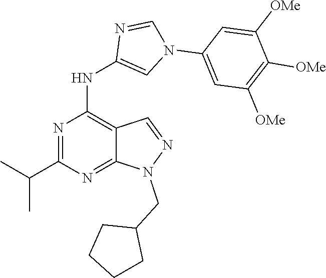 [SMILES: COc1cc(-n2cnc(Nc3nc(C(C)C)nc4c3cnn4CC3CCCC3)c2)cc(OC)c1OC]

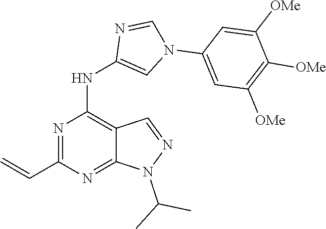 [SMILES: C=Cc1nc(Nc2cn(-c3cc(OC)c(OC)c(OC)c3)cn2)c2cnn(C(C)C)c2n1]

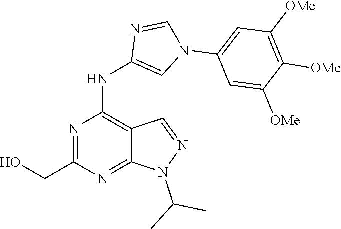 [SMILES: COc1cc(-n2cnc(Nc3nc(CO)nc4c3cnn4C(C)C)c2)cc(OC)c1OC]

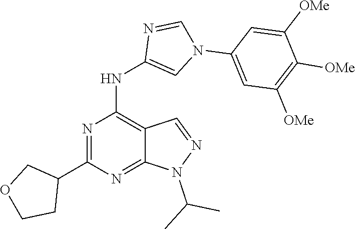 [SMILES: COc1cc(-n2cnc(Nc3nc(C4CCOC4)nc4c3cnn4C(C)C)c2)cc(OC)c1OC]

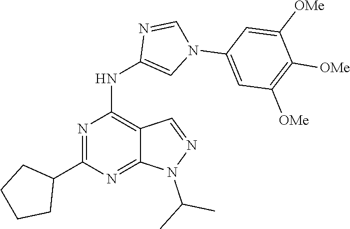 [SMILES: COc1cc(-n2cnc(Nc3nc(C4CCCC4)nc4c3cnn4C(C)C)c2)cc(OC)c1OC]

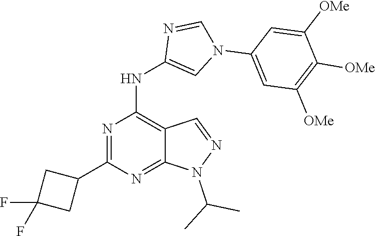 [SMILES: COc1cc(-n2cnc(Nc3nc(C4CC(F)(F)C4)nc4c3cnn4C(C)C)c2)cc(OC)c1OC]

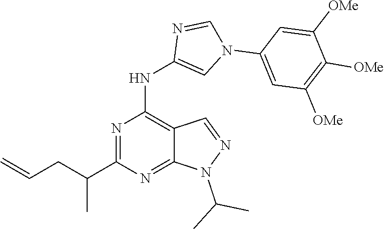 [SMILES: C=CCC(C)c1nc(Nc2cn(-c3cc(OC)c(OC)c(OC)c3)cn2)c2cnn(C(C)C)c2n1]

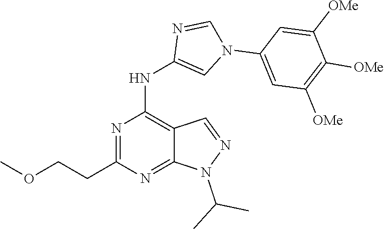 [SMILES: COCCc1nc(Nc2cn(-c3cc(OC)c(OC)c(OC)c3)cn2)c2cnn(C(C)C)c2n1]

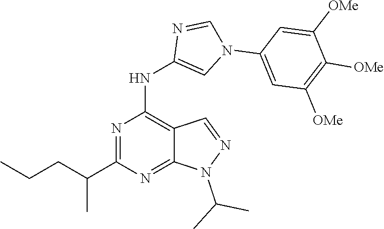 [SMILES: CCCC(C)c1nc(Nc2cn(-c3cc(OC)c(OC)c(OC)c3)cn2)c2cnn(C(C)C)c2n1]

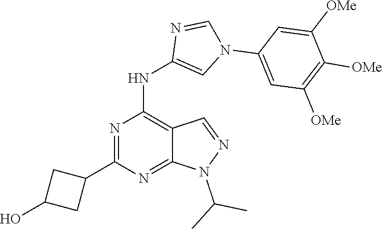 [SMILES: COc1cc(-n2cnc(Nc3nc(C4CC(O)C4)nc4c3cnn4C(C)C)c2)cc(OC)c1OC]

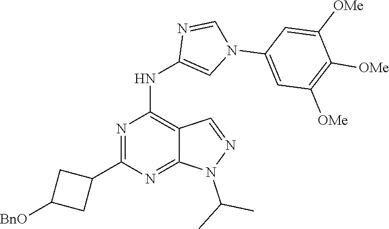 [SMILES: COc1cc(-n2cnc(Nc3nc(C4CC(OCc5ccccc5)C4)nc4c3cnn4C(C)C)c2)cc(OC)c1OC]

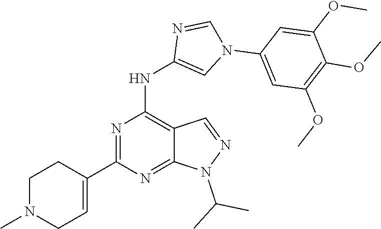

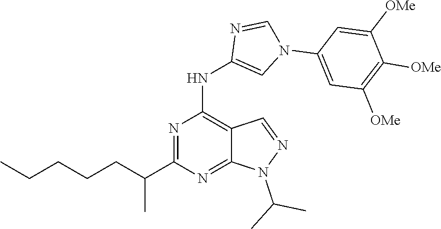 [SMILES: CCCCCC(C)c1nc(Nc2cn(-c3cc(OC)c(OC)c(OC)c3)cn2)c2cnn(C(C)C)c2n1]

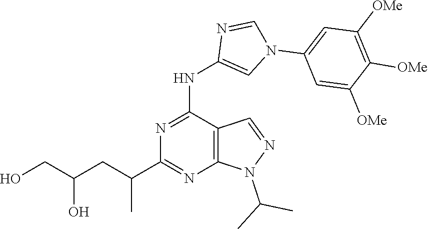 [SMILES: COc1cc(-n2cnc(Nc3nc(C(C)CC(O)CO)nc4c3cnn4C(C)C)c2)cc(OC)c1OC]

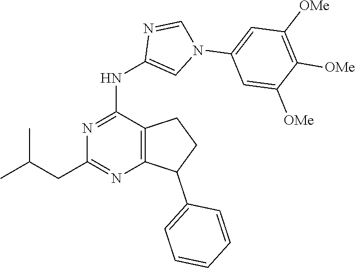 [SMILES: COc1cc(-n2cnc(Nc3nc(CC(C)C)nc4c3CCC4c3ccccc3)c2)cc(OC)c1OC]

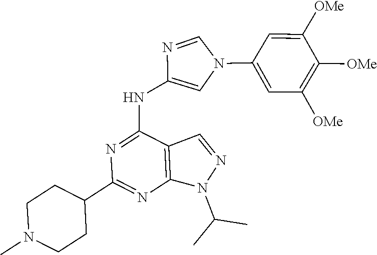 [SMILES: COc1cc(-n2cnc(Nc3nc(C4CCN(C)CC4)nc4c3cnn4C(C)C)c2)cc(OC)c1OC]

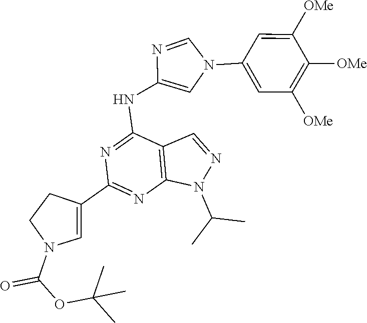 [SMILES: COc1cc(-n2cnc(Nc3nc(C4=CN(C(=O)OC(C)(C)C)CC4)nc4c3cnn4C(C)C)c2)cc(OC)c1OC]

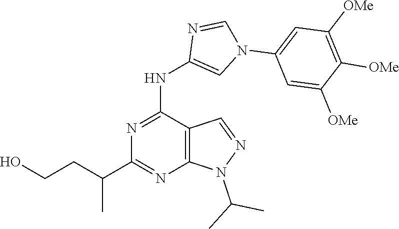 [SMILES: COc1cc(-n2cnc(Nc3nc(C(C)CCO)nc4c3cnn4C(C)C)c2)cc(OC)c1OC]

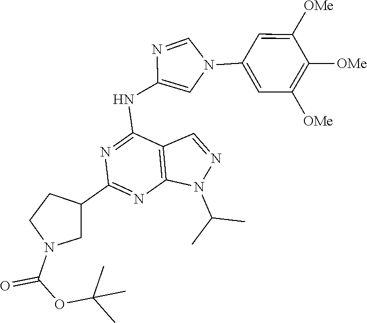 [SMILES: COc1cc(-n2cnc(Nc3nc(C4CCN(C(=O)OC(C)(C)C)C4)nc4c3cnn4C(C)C)c2)cc(OC)c1OC]

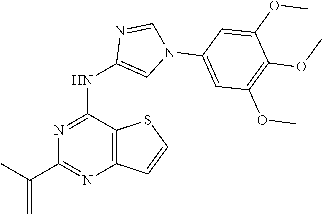 [SMILES: C=C(C)c1nc(Nc2cn(-c3cc(OC)c(OC)c(OC)c3)cn2)c2sccc2n1]

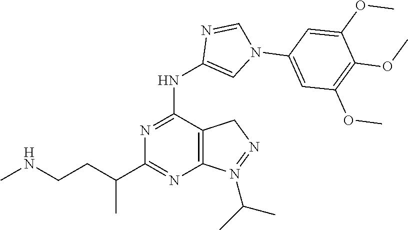

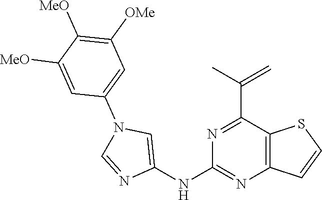 [SMILES: C=C(C)c1nc(Nc2cn(-c3cc(OC)c(OC)c(OC)c3)cn2)nc2ccsc12]

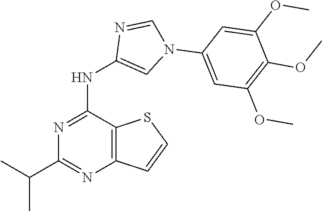 [SMILES: COc1cc(-n2cnc(Nc3nc(C(C)C)nc4ccsc34)c2)cc(OC)c1OC]

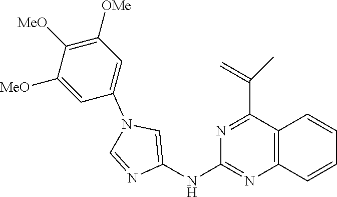 [SMILES: C=C(C)c1nc(Nc2cn(-c3cc(OC)c(OC)c(OC)c3)cn2)nc2ccccc12]

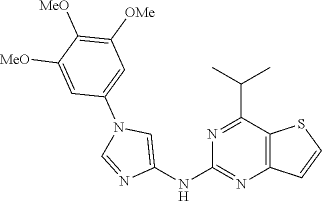 [SMILES: COc1cc(-n2cnc(Nc3nc(C(C)C)c4sccc4n3)c2)cc(OC)c1OC]

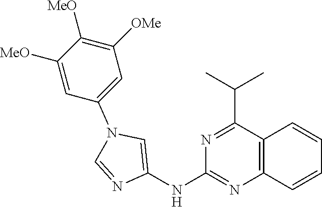 [SMILES: COc1cc(-n2cnc(Nc3nc(C(C)C)c4ccccc4n3)c2)cc(OC)c1OC]

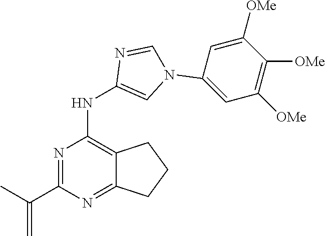 [SMILES: C=C(C)c1nc2c(c(Nc3cn(-c4cc(OC)c(OC)c(OC)c4)cn3)n1)CCC2]

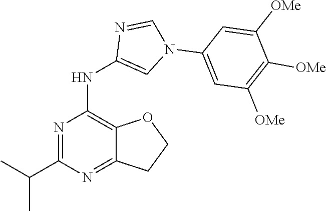 [SMILES: COc1cc(-n2cnc(Nc3nc(C(C)C)nc4c3OCC4)c2)cc(OC)c1OC]

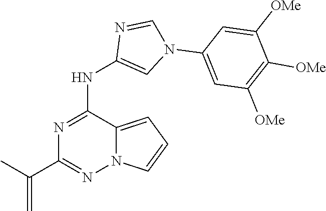 [SMILES: C=C(C)c1nc(Nc2cn(-c3cc(OC)c(OC)c(OC)c3)cn2)c2cccn2n1]

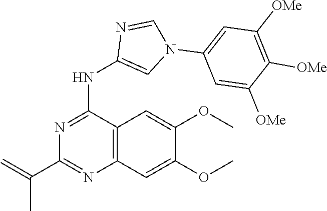 [SMILES: C=C(C)c1nc(Nc2cn(-c3cc(OC)c(OC)c(OC)c3)cn2)c2cc(OC)c(OC)cc2n1]

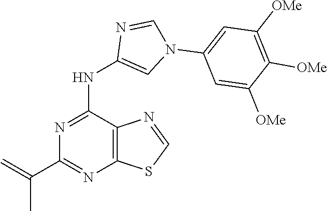 [SMILES: C=C(C)c1nc(Nc2cn(-c3cc(OC)c(OC)c(OC)c3)cn2)c2ncsc2n1]

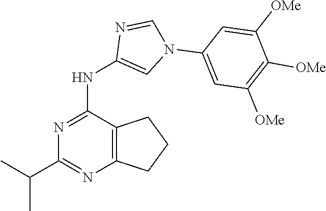 [SMILES: COc1cc(-n2cnc(Nc3nc(C(C)C)nc4c3CCC4)c2)cc(OC)c1OC]

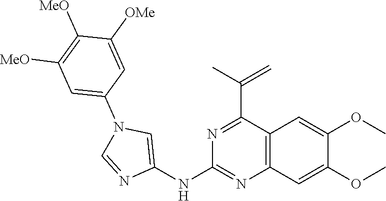 [SMILES: C=C(C)c1nc(Nc2cn(-c3cc(OC)c(OC)c(OC)c3)cn2)nc2cc(OC)c(OC)cc12]

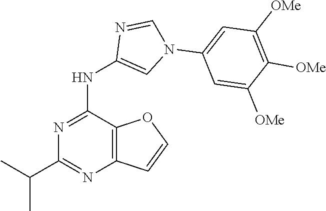 [SMILES: COc1cc(-n2cnc(Nc3nc(C(C)C)nc4ccoc34)c2)cc(OC)c1OC]

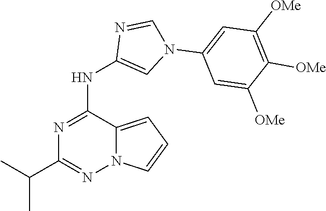 [SMILES: COc1cc(-n2cnc(Nc3nc(C(C)C)nn4cccc34)c2)cc(OC)c1OC]

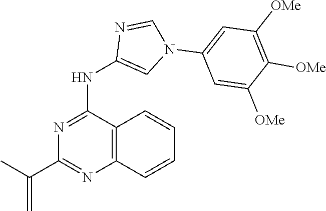 [SMILES: C=C(C)c1nc(Nc2cn(-c3cc(OC)c(OC)c(OC)c3)cn2)c2ccccc2n1]

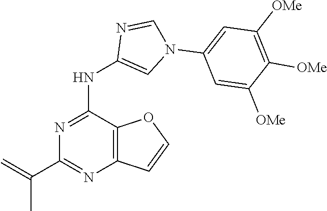 [SMILES: C=C(C)c1nc(Nc2cn(-c3cc(OC)c(OC)c(OC)c3)cn2)c2occc2n1]

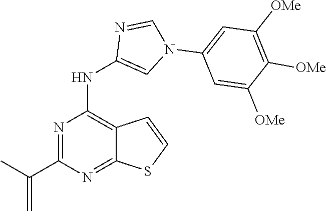 [SMILES: C=C(C)c1nc(Nc2cn(-c3cc(OC)c(OC)c(OC)c3)cn2)c2ccsc2n1]

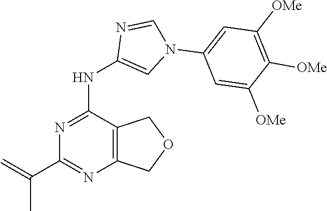 [SMILES: C=C(C)c1nc2c(c(Nc3cn(-c4cc(OC)c(OC)c(OC)c4)cn3)n1)COC2]

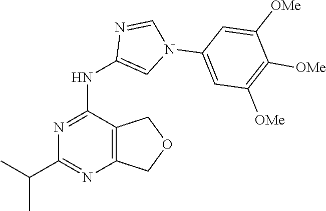 [SMILES: COc1cc(-n2cnc(Nc3nc(C(C)C)nc4c3COC4)c2)cc(OC)c1OC]

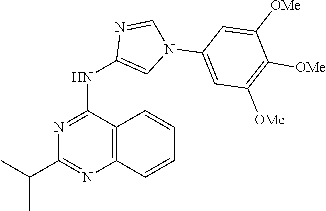 [SMILES: COc1cc(-n2cnc(Nc3nc(C(C)C)nc4ccccc34)c2)cc(OC)c1OC]

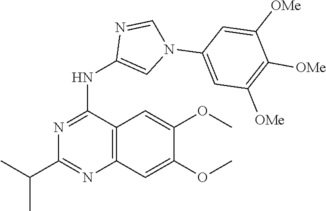 [SMILES: COc1cc2nc(C(C)C)nc(Nc3cn(-c4cc(OC)c(OC)c(OC)c4)cn3)c2cc1OC]

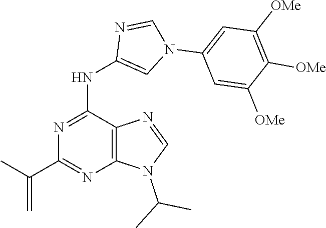 [SMILES: C=C(C)c1nc(Nc2cn(-c3cc(OC)c(OC)c(OC)c3)cn2)c2ncn(C(C)C)c2n1]

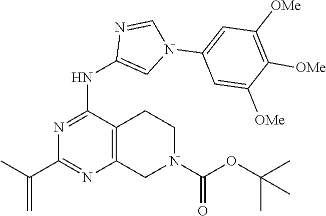 [SMILES: C=C(C)c1nc2c(c(Nc3cn(-c4cc(OC)c(OC)c(OC)c4)cn3)n1)CCN(C(=O)OC(C)(C)C)C2]

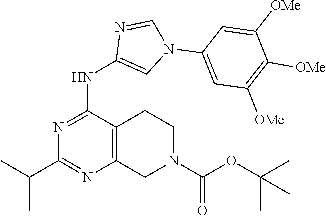 [SMILES: COc1cc(-n2cnc(Nc3nc(C(C)C)nc4c3CCN(C(=O)OC(C)(C)C)C4)c2)cc(OC)c1OC]

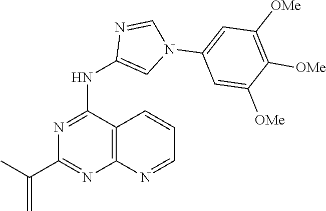 [SMILES: C=C(C)c1nc(Nc2cn(-c3cc(OC)c(OC)c(OC)c3)cn2)c2cccnc2n1]

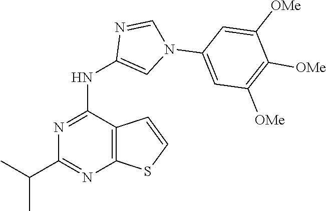 [SMILES: COc1cc(-n2cnc(Nc3nc(C(C)C)nc4sccc34)c2)cc(OC)c1OC]

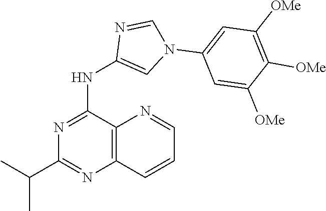 [SMILES: COc1cc(-n2cnc(Nc3nc(C(C)C)nc4cccnc34)c2)cc(OC)c1OC]

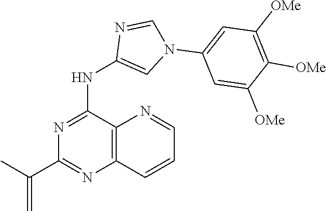 [SMILES: C=C(C)c1nc(Nc2cn(-c3cc(OC)c(OC)c(OC)c3)cn2)c2ncccc2n1]

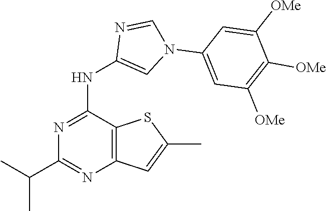 [SMILES: COc1cc(-n2cnc(Nc3nc(C(C)C)nc4cc(C)sc34)c2)cc(OC)c1OC]

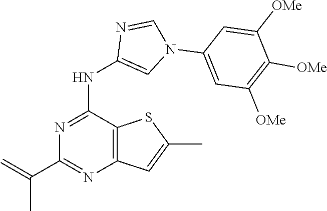 [SMILES: C=C(C)c1nc(Nc2cn(-c3cc(OC)c(OC)c(OC)c3)cn2)c2sc(C)cc2n1]

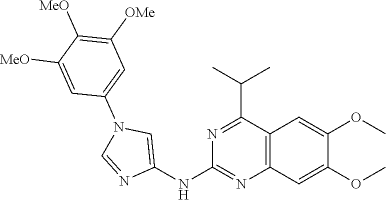 [SMILES: COc1cc2nc(Nc3cn(-c4cc(OC)c(OC)c(OC)c4)cn3)nc(C(C)C)c2cc1OC]

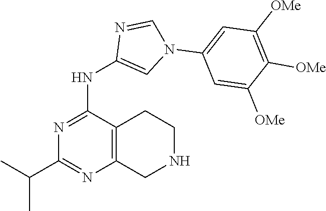 [SMILES: COc1cc(-n2cnc(Nc3nc(C(C)C)nc4c3CCNC4)c2)cc(OC)c1OC]

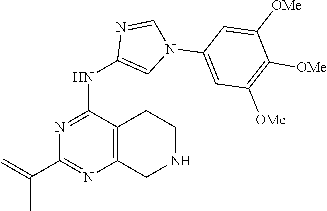 [SMILES: C=C(C)c1nc2c(c(Nc3cn(-c4cc(OC)c(OC)c(OC)c4)cn3)n1)CCNC2]

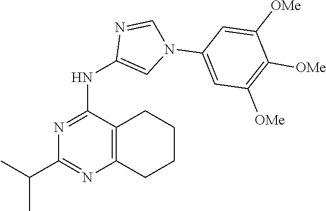 [SMILES: COc1cc(-n2cnc(Nc3nc(C(C)C)nc4c3CCCC4)c2)cc(OC)c1OC]

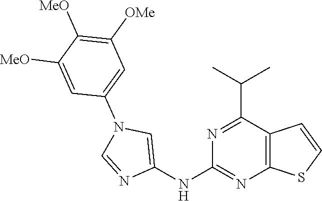 [SMILES: COc1cc(-n2cnc(Nc3nc(C(C)C)c4ccsc4n3)c2)cc(OC)c1OC]

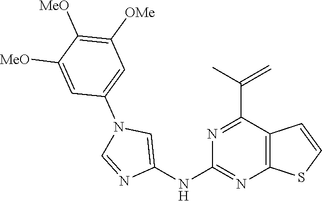 [SMILES: C=C(C)c1nc(Nc2cn(-c3cc(OC)c(OC)c(OC)c3)cn2)nc2sccc12]

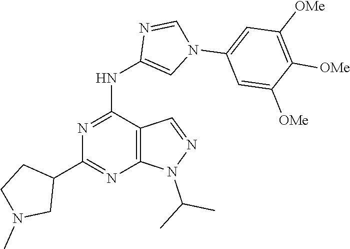 [SMILES: COc1cc(-n2cnc(Nc3nc(C4CCN(C)C4)nc4c3cnn4C(C)C)c2)cc(OC)c1OC]

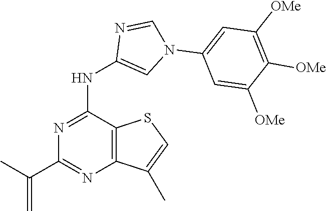 [SMILES: C=C(C)c1nc(Nc2cn(-c3cc(OC)c(OC)c(OC)c3)cn2)c2scc(C)c2n1]

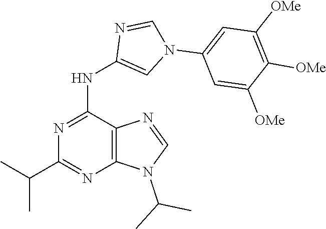 [SMILES: COc1cc(-n2cnc(Nc3nc(C(C)C)nc4c3ncn4C(C)C)c2)cc(OC)c1OC]

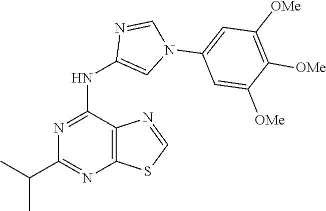 [SMILES: COc1cc(-n2cnc(Nc3nc(C(C)C)nc4scnc34)c2)cc(OC)c1OC]

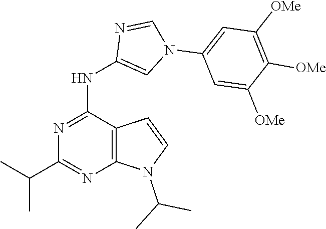 [SMILES: COc1cc(-n2cnc(Nc3nc(C(C)C)nc4c3ccn4C(C)C)c2)cc(OC)c1OC]

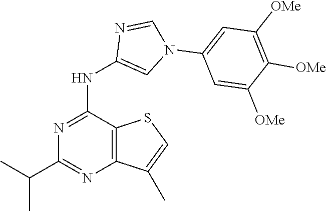 [SMILES: COc1cc(-n2cnc(Nc3nc(C(C)C)nc4c(C)csc34)c2)cc(OC)c1OC]

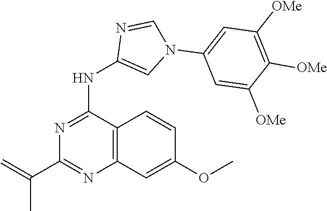 [SMILES: C=C(C)c1nc(Nc2cn(-c3cc(OC)c(OC)c(OC)c3)cn2)c2ccc(OC)cc2n1]

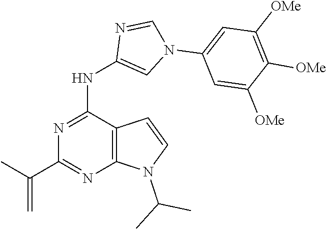 [SMILES: C=C(C)c1nc(Nc2cn(-c3cc(OC)c(OC)c(OC)c3)cn2)c2ccn(C(C)C)c2n1]

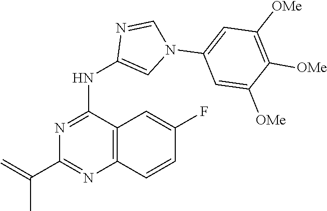 [SMILES: C=C(C)c1nc(Nc2cn(-c3cc(OC)c(OC)c(OC)c3)cn2)c2cc(F)ccc2n1]

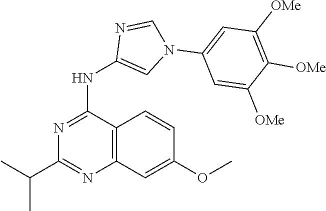 [SMILES: COc1ccc2c(Nc3cn(-c4cc(OC)c(OC)c(OC)c4)cn3)nc(C(C)C)nc2c1]

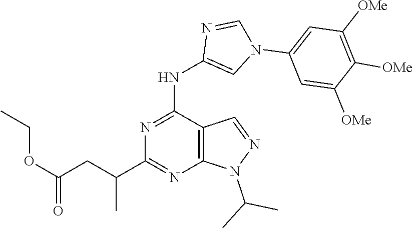 [SMILES: CCOC(=O)CC(C)c1nc(Nc2cn(-c3cc(OC)c(OC)c(OC)c3)cn2)c2cnn(C(C)C)c2n1]

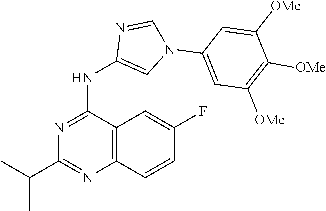 [SMILES: COc1cc(-n2cnc(Nc3nc(C(C)C)nc4ccc(F)cc34)c2)cc(OC)c1OC]

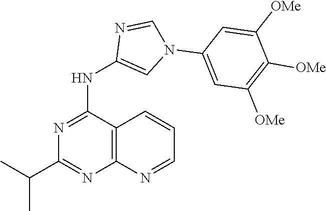 [SMILES: COc1cc(-n2cnc(Nc3nc(C(C)C)nc4ncccc34)c2)cc(OC)c1OC]

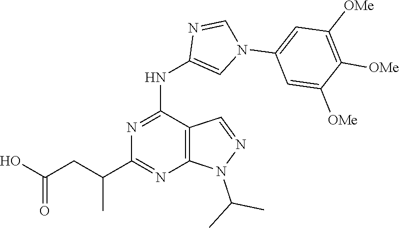 [SMILES: COc1cc(-n2cnc(Nc3nc(C(C)CC(=O)O)nc4c3cnn4C(C)C)c2)cc(OC)c1OC]

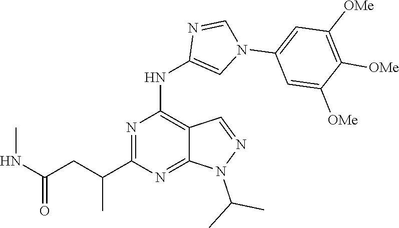 [SMILES: CNC(=O)CC(C)c1nc(Nc2cn(-c3cc(OC)c(OC)c(OC)c3)cn2)c2cnn(C(C)C)c2n1]

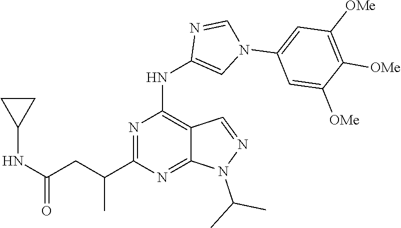 [SMILES: COc1cc(-n2cnc(Nc3nc(C(C)CC(=O)NC4CC4)nc4c3cnn4C(C)C)c2)cc(OC)c1OC]

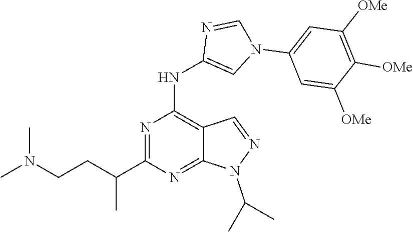 [SMILES: COc1cc(-n2cnc(Nc3nc(C(C)CCN(C)C)nc4c3cnn4C(C)C)c2)cc(OC)c1OC]

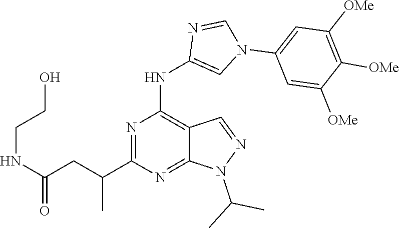 [SMILES: COc1cc(-n2cnc(Nc3nc(C(C)CC(=O)NCCO)nc4c3cnn4C(C)C)c2)cc(OC)c1OC]

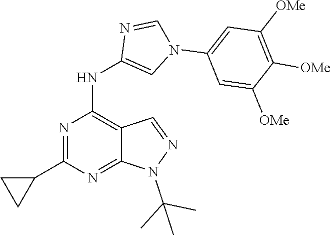 [SMILES: COc1cc(-n2cnc(Nc3nc(C4CC4)nc4c3cnn4C(C)(C)C)c2)cc(OC)c1OC]

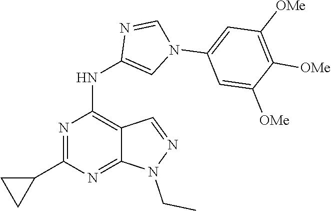 [SMILES: CCn1ncc2c(Nc3cn(-c4cc(OC)c(OC)c(OC)c4)cn3)nc(C3CC3)nc21]

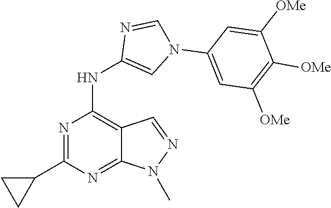 [SMILES: COc1cc(-n2cnc(Nc3nc(C4CC4)nc4c3cnn4C)c2)cc(OC)c1OC]

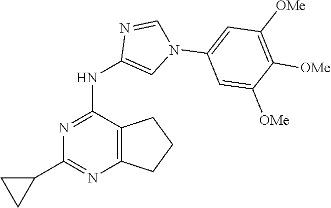 [SMILES: COc1cc(-n2cnc(Nc3nc(C4CC4)nc4c3CCC4)c2)cc(OC)c1OC]

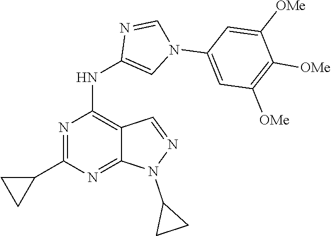 [SMILES: COc1cc(-n2cnc(Nc3nc(C4CC4)nc4c3cnn4C3CC3)c2)cc(OC)c1OC]

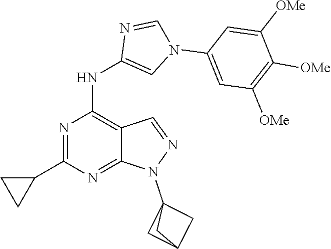 [SMILES: COc1cc(-n2cnc(Nc3nc(C4CC4)nc4c3cnn4C34CC(C3)C4)c2)cc(OC)c1OC]

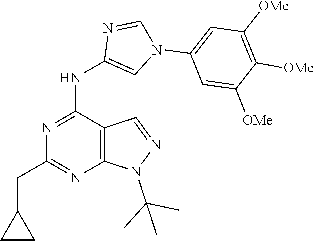 [SMILES: COc1cc(-n2cnc(Nc3nc(CC4CC4)nc4c3cnn4C(C)(C)C)c2)cc(OC)c1OC]

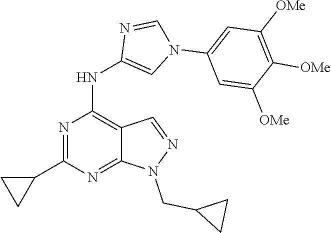 [SMILES: COc1cc(-n2cnc(Nc3nc(C4CC4)nc4c3cnn4CC3CC3)c2)cc(OC)c1OC]

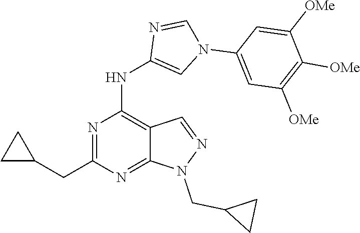 [SMILES: COc1cc(-n2cnc(Nc3nc(CC4CC4)nc4c3cnn4CC3CC3)c2)cc(OC)c1OC]

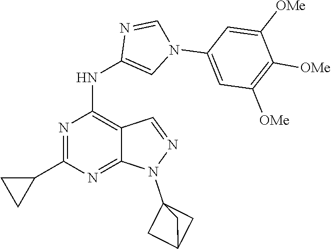 [SMILES: COc1cc(-n2cnc(Nc3nc(C4CC4)nc4c3cnn4C34CC(C3)C4)c2)cc(OC)c1OC]

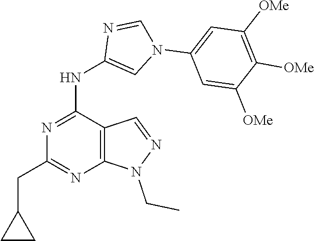 [SMILES: CCn1ncc2c(Nc3cn(-c4cc(OC)c(OC)c(OC)c4)cn3)nc(CC3CC3)nc21]

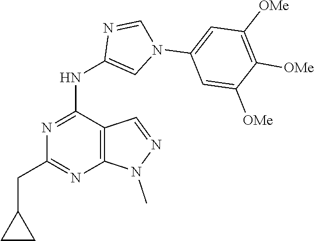 [SMILES: COc1cc(-n2cnc(Nc3nc(CC4CC4)nc4c3cnn4C)c2)cc(OC)c1OC]

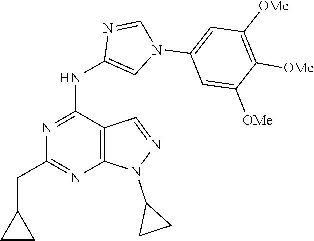 [SMILES: COc1cc(-n2cnc(Nc3nc(CC4CC4)nc4c3cnn4C3CC3)c2)cc(OC)c1OC]

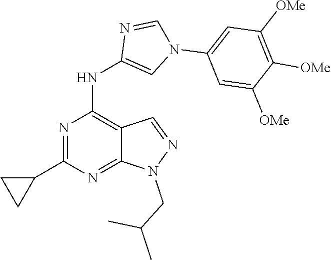 [SMILES: COc1cc(-n2cnc(Nc3nc(C4CC4)nc4c3cnn4CC(C)C)c2)cc(OC)c1OC]

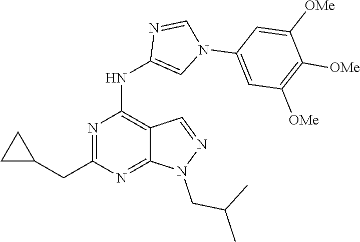 [SMILES: COc1cc(-n2cnc(Nc3nc(CC4CC4)nc4c3cnn4CC(C)C)c2)cc(OC)c1OC]

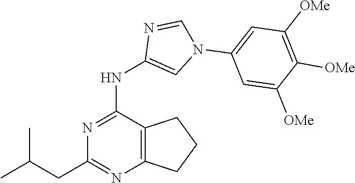 [SMILES: COc1cc(-n2cnc(Nc3nc(CC(C)C)nc4c3CCC4)c2)cc(OC)c1OC]

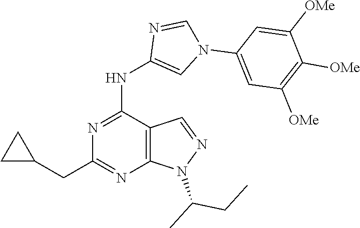 [SMILES: CC[C@H](C)n1ncc2c(Nc3cn(-c4cc(OC)c(OC)c(OC)c4)cn3)nc(CC3CC3)nc21]

51. A compound represented by formula (III) or formula (IV): 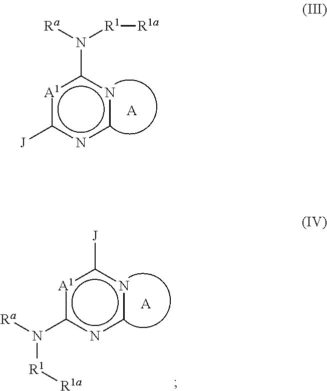
or a pharmaceutically acceptable salt thereof;
wherein:
A is a fused optionally substituted aromatic ring, heteroaromatic ring, cycloalkyl ring, cycloalkenyl ring, heterocycloalkyl ring, or heterocycloalkenyl ring;
A¹ is CH or N;
R^{a} represents H or alkyl;
R¹ represents heteroarylene;
R^{1a} represents H or optionally substituted —C(O)alkyl, —C(O)aryl, —C(O)heteroaryl, —C(O)O(alkyl), —C(O)(heterocyclyl), —C(O)NR^{x}R^{y}, alkyl, cycloalkyl, cycloalkenyl, heterocycloalkyl, aryl, or heteroaryl;
J represents optionally substituted alkyl, alkenyl, haloalkyl, hydroxyalkyl, alkoxyalkyl, cycloalkyl, (cycloalkyl)alkyl, heterocycloalkyl, (heterocycloalkyl)alkyl, or heterocycloalkenyl; and
R^{x} and R^{y} each independently represent H, alkyl, aralkyl, heteroaralkyl, aryl, heteroaryl, cycloalkyl, heterocycloalkyl, (cycloalkyl)alkyl, (heterocycloalkyl)alkyl, or hydroxyalkyl.

52. The compound of claim 51, having the structure of formula (IIIa) or (IVa): 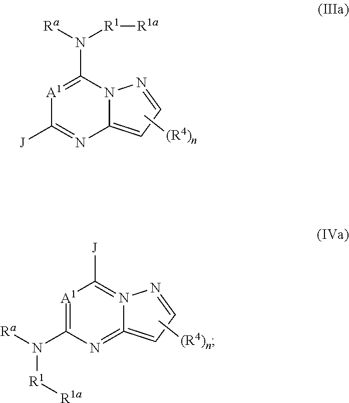
wherein:
R⁴, independently for each occurrence, represents halo, cyano, —CH₂C(O)NH₂, —C(O)R⁵, —C(O)OR⁵, —S(O)₂R⁵, or optionally substituted alkyl, alkenyl, haloalkyl, hydroxyalkyl, aminoalkyl, alkoxy, aryl, aralkyl, heteroaryl, heteroaralkyl, heterocycloalkyl, heterocycloalkenyl, (heterocycloalkyl)alkyl, cycloalkyl, (cycloalkyl)alkyl, halocycloalkyl, hydroxycycloalkyl, aminocycloalkyl, aryloxy, heteroaryloxy, arylalkyloxy, or heteroarylalkyloxy;
R⁵, independently for each occurrence, represents optionally substituted alkyl, aralkyl, aryl, heteroaralkyl, heteroaryl, cycloalkyl, heterocycloalkyl, (cycloalkyl)alkyl, or (heterocycloalkyl)alkyl; and
n is an integer from 0-2.

53. The compound of claim 51 or 52, wherein A¹ is CH.

54. The compound of claim 51 or 52, wherein A¹ is N.

55. The compound of any one of claims 52-54, wherein n is 0 or 1.

56. The compound of any one of claims 52-55, wherein R⁴, if present, is alkyl.

57. The compound of any one of claims 52-56, wherein R^{a} is H.

58. The compound of any one of claims 52-57, wherein R¹ is a nitrogen-containing heteroarylene.

59. The compound of any one of claims 52-58, wherein R¹ is a 5-membered nitrogen-containing heteroarylene.

60. The compound of any one of claims 52-59, wherein R¹ is imidazolene.

61. The compound of any one of claims 52-60, wherein —R¹—R^{1a} represents

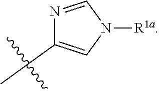

62. The compound of any one of claims 52-61, wherein R^{1a} is optionally substituted phenyl.

63. The compound of any one of claims 52-62, wherein R^{1a} is phenyl, substituted by one or more occurrences of alkoxy.

64. The compound of claim 63, wherein RIB is 3,4,5-trimethoxyphenyl.

65. The compound of any one of claims 52-64, wherein J represents optionally substituted cycloalkyl.

66. The compound of claim 51, or a pharmaceutically acceptable salt thereof, selected from the following table:

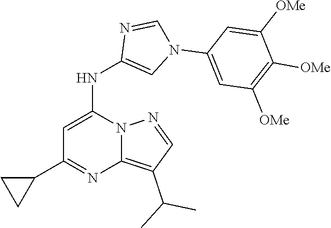 [SMILES: COc1cc(-n2cnc(Nc3cc(C4CC4)nc4c(C(C)C)cnn34)c2)cc(OC)c1OC]

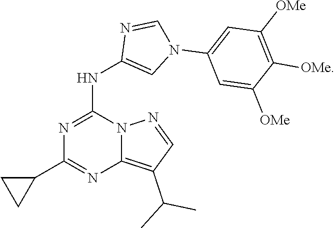 [SMILES: COc1cc(-n2cnc(Nc3nc(C4CC4)nc4c(C(C)C)cnn34)c2)cc(OC)c1OC]

67. A pharmaceutical composition, comprising a compound of any one of claims 1-66, or a pharmaceutically acceptable salt thereof; and a pharmaceutically acceptable carrier.

68. A method of inhibiting ALK2 kinase, comprising administering to a subject in need thereof an effective amount of a compound of any one of claims 1-66, or a pharmaceutically acceptable salt thereof.

69. A method of treating fibrodysplasia ossificans progressiva, comprising administering to a subject in need thereof a therapeutically effective amount of a compound of any one of claims 1-66, or a pharmaceutically acceptable salt thereof.

70. A method of treating cancer, comprising administering to a subject in need thereof a therapeutically effective amount of a compound of any one of claims 1-66, or a pharmaceutically acceptable salt thereof.

71. The method of claim 70, wherein the cancer is a glioma.

72. The method of claim 71, wherein the glioma is diffuse intrinsic pontine glioma.

73. A method of treating anemia associated with high hepcidin, Iron Refractory Iron Deficiency Anemia (IRIDA), anemia of chronic diseases, cancer-related anemia, chemotherapy-associated anemia, anemia of inflammation, or hepcidin-producing adenoma, comprising administering to a subject in need thereof a therapeutically effective amount of a compound of any one of claims 1-66, or a pharmaceutically acceptable salt thereof.

74. A method of treating spondyloarthritis (SpA), comprising administering to a subject in need thereof a therapeutically effective amount of a compound of any one of claims 1-66, or a pharmaceutically acceptable salt thereof.
